(19) United States
(12) Patent Application Publication
(10) Pub. No.: US 2011/0152246 A1
(43) Pub. Date: Jun. 23, 2011

(54) NOVEL INHIBITORS OF HEPATITIS C VIRUS REPLICATION

(75) Inventors: Brad Buckman, Oakland, CA (US); John B. Nicholas, Redwood City, CA (US); Vladimir Serebryany, Burlingame, CA (US); Scott D. Seiwert, Pacifica, CA (US)

(73) Assignee: InterMune, Inc., Brisbane, CA (US)

(21) Appl. No.: 12/970,492

(22) Filed: Dec. 16, 2010

Related U.S. Application Data
(60) Provisional application No. 61/288,251, filed on Dec. 18, 2009.
(60) Provisional application No. 61/309,793, filed on Mar. 2, 2010.
(60) Provisional application No. 61/321,077, filed on Apr. 5, 2010.
(60) Provisional application No. 61/345,222, filed on May 17, 2010.
(60) Provisional application No. 61/345,553, filed on May 17, 2010.
(60) Provisional application No. 61/354,671, filed on Jun. 14, 2010.
(60) Provisional application No. 61/361,328, filed on Jul. 2, 2010.
(60) Provisional application No. 61/382,872, filed on Sep. 14, 2010.
(60) Provisional application No. 61/405,138, filed on Oct. 20, 2010.

(51) Int. Cl. A61K 31/553; C07D 403/14; C07D 277/62; C07D 233/64; C07D 263/56; C07D 221/06; C07D 233/54; C07D 267/18; A61K 31/4184; A61K 31/428; A61K 31/4178; A61K 31/421; A61K 31/497; A61K 31/435; A61K 31/451; C07D 279/18; A61K 31/5415; A61P 31/14

(52) U.S. Cl. 514/211.04; 548/306.1; 548/152; 548/311.4; 548/217; 544/370; 544/295; 546/101; 548/313.1; 548/311.7; 540/488; 514/394; 514/367; 514/397; 514/375; 514/254.05; 514/252.2; 514/290; 514/326; 544/35; 514/227.8

(57) ABSTRACT

The embodiments provide compounds of the general Formulae I, II, III, IV, or V as well as compositions, including pharmaceutical compositions, comprising a subject compound. The embodiments further provide treatment methods, including methods of treating a hepatitis C virus infection and methods of treating liver fibrosis, the methods generally involving administering to an individual in need thereof an effective amount of a subject compound or composition.

### RELATED APPLICATIONS

This application claims the benefit of U.S. Provisional Application Nos. 61/288,251, filed Dec. 18, 2009; 61/309,793, filed Mar. 2, 2010; 61/321,077, filed Apr. 5, 2010; 61/345,222, filed May 17, 2010; 61/345,553, filed May 17, 2010; 61/354,671, filed Jun. 14, 2010; 61/361,328, filed Jul. 2, 2010; 61/382,872, filed Sep. 14, 2010; and 61/405,138, filed Oct. 20, 2010; all of which are incorporated herein by reference in their entirety.

### BACKGROUND OF THE INVENTION

1. Field of the Invention

The embodiments described herein relate to compounds, processes for their synthesis, compositions and methods for the therapeutic use of the compounds, such as for treating hepatitis C virus (HCV) infection.

2. Description of the Related Art

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

Antiviral therapy of chronic hepatitis C has evolved rapidly over the last decade, with significant improvements seen in the efficacy of treatment. Nevertheless, even with combination therapy using pegylated IFN-α plus ribavirin, 40% to 50% of patients fail therapy; they are nonresponders or relapsers. These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, this will result in a substantial increase in cirrhosis-related morbidity and mortality among patients infected between the years of 1965-1985.

HCV is an enveloped positive strand RNA virus in the Flaviviridae family. The single strand HCV RNA genome is believed to be approximately 9500 nucleotides in length and has a single open reading frame (ORF) encoding a single large polyprotein of about 3000 amino acids. In infected cells, it is believed that this polyprotein is cleaved at multiple sites by cellular and viral proteases to produce the structural and non-structural (NS) proteins of the virus. In the case of HCV, the generation of mature nonstructural proteins (NS2, NS3, NS4, NS4A, NS4B, NS5A, and NS5B) is believed to be effected by two viral proteases. The first viral protease is believed to cleave at the NS2-NS3 junction of the polyprotein. The second viral protease is believed to be a serine protease contained within the N-terminal region of NS3 (herein referred to as “NS3 protease”). NS3 protease is believed to mediate all of the subsequent cleavage events at sites downstream relative to the position of NS3 in the polyprotein (i.e., sites located between the C-terminus of NS3 and the C-terminus of the polyprotein). NS3 protease exhibits activity both in cis, at the NS3-NS4 cleavage site, and in trans, for the remaining NS4A-NS4B, NS4B-NS5A, and NS5A-NS5B sites. The NS4A protein is believed to serve multiple functions, acting as a cofactor for the NS3 protease and possibly assisting in the membrane localization of NS3 and other viral replicase components. Apparently, the formation of the complex between NS3 and NS4A may be necessary for NS3-mediated processing events and enhances proteolytic efficiency at all sites recognized by NS3. The NS3 protease also appears to exhibit nucleoside triphosphatase and RNA helicase activities. NS5B is believed to be an RNA-dependent RNA polymerase involved in the replication of HCV RNA. In addition, compounds that inhibit the action of NS5A in viral replication are potentially useful for the treatment of HCV.

### SUMMARY OF THE INVENTION

Some embodiments include a compound having the structure of Formula I:

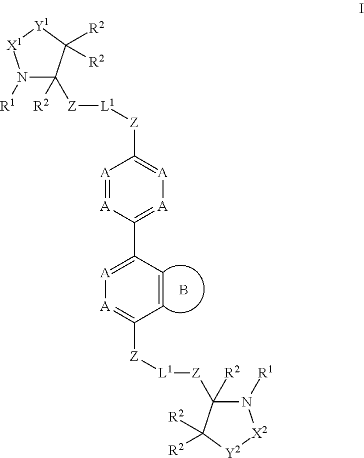

or a pharmaceutically acceptable salt thereof,

wherein:

each R¹ is separately selected from the group consisting of hydrogen, R^{1a}S(O₂)—, R^{1a}C(═O)— and R^{1a}C(═S)—;

each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;

each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;

each R^{e}R^{f}N is separately selected, wherein R^{e} and R^{f} are each separately selected from the group consisting of hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;

each R^{x}R^{y}N is separately selected, wherein R^{x} and R^{y} are each separately selected from the group consisting of hydrogen, alkylOC(═O)—, C₁₋₆alkyl, C₁₋₆alkylC(═O)—, aryl, arylalkyl, cycloalkyl, and heterocyclyl;

each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is

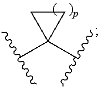

each R^{3a} is separately selected from the group consisting of hydrogen, and optionally substituted C₁₋₆alkyl;

each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;

each R^{4a}R^{4b}N is separately selected, wherein R^{4a} and R^{4b} are each separately selected from the group consisting of hydrogen, optionally substituted C₁₋₆alkyl, and aryl(CH₂)ₙ—;

each R^{5a} is separately selected from the group consisting of optionally substituted C₁₋₆alkyl, and aryl(CH₂)ₙ—;

each R^{6a} is separately selected from the group consisting of optionally substituted C₁₋₆alkyl, and aryl(CH₂)ₙ—;

X¹ is (C(R²)₂)_{q},

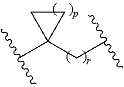

or X₁ is null;

Y¹ is selected from O (oxygen), S (sulfur), S(O), SO₂, NR², and C(R²)₂ with the proviso that when X₁ is null Y₁ is C(R²)₂;

X² is (C(R²)₂)_{q},

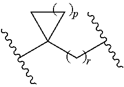

or X² is null;

Y² is selected from O (oxygen), S (sulfur), S(O), SO₂, NR², and C(R²)₂ with the proviso that when X² is null Y² is C(R²)₂;

each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups;

each Z is separately selected, wherein Z is selected from the group consisting of O (oxygen) and CH₂, or Z is null;

each A is separately selected from the group consisting of CR³ and N (nitrogen);

each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 9 halo and up to 5 hydroxy;

each L₁ is separately selected from the group consisting of

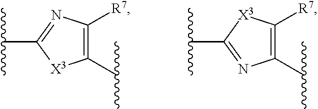

—C(═O)(CH₂)ₘOC(═O)—, —C(CF₃)₂NR^{2c}—, and

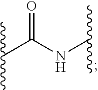

each X³ is separately selected from the group consisting of NH, NC₁₋₆alkyl, O (oxygen), and S (sulfur);

each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo;

each R^{a}R^{b}N is separately selected, wherein R^{a} and R^{b} are each separately selected from the group consisting of hydrogen, C₂₋₆alkenyl, and C₁₋₆alkyl;

each m separately is 1 or 2;

each n separately is 0, 1 or 2;

each p separately is 1, 2, 3 or 4;

each q separately is 1, 2, 3, 4 or 5;

each r separately is 0, 1, 2, 3, or 4;

B is a fused optionally substituted saturated or unsaturated three- to seven-membered carbocyclic ring, a fused optionally substituted saturated or unsaturated three- to seven-membered heterocyclic ring, or a fused optionally substituted five- or six-membered heteroaryl ring, each optionally substituted with one or more R⁴; and

each R⁴ is separately selected from the group consisting of C₁₋₆alkoxy; C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, C₁₋₆alkyl optionally substituted with up to 9 halo and up to 5 hydroxy, or optionally two geminal R⁴ are together oxo.

In some embodiments of Formula I, each R¹ is separately selected from the group consisting of hydrogen and R^{1a}C(═O)— and R^{1a}C(═S)—;

each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, (R^{c}R^{d}N)(CH═CH)ₘ—, (R^{c}R^{d}N)alkyl, (R^{c}R^{d}N)C(═O)—, C₁₋₆alkoxy optionally substituted with up to 5 halo, and C₁₋₆alkyl optionally substituted with up to 5 halo;

each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 5 halo, and C₁₋₆alkyl optionally substituted with up to 5 halo;

each R^{2a} is separately selected from the group consisting of hydrogen, C₁₋₆alkyl, aryl(CH₂)ₙ—, and heteroaryl(CH₂)ₙ—;

each R^{3a} is separately selected from the group consisting of hydrogen, and C₁₋₆alkyl;

each R^{3b} is separately selected from the group consisting of C₁₋₆alkyl, —(CH₂)ₙC(═O)NR^{4a}R^{4b}, —(CH₂)ₙC(═O)OR^{5a}, and —(CH₂)ₙC(═O)R^{6a};

each R^{4a}R^{4b}N is separately selected, wherein R^{4a} and R^{4b} are each separately selected from the group consisting of hydrogen, C₁₋₆alkyl, and aryl(CH₂)ₙ—;

each R^{5a} is separately selected from the group consisting of C₁₋₆alkyl, and aryl(CH₂)ₙ—;

each R^{6a} is separately selected from the group consisting of C₁₋₆alkyl, and aryl (CH₂)ₙ—;

X¹ is C(R²)₂, or X₁ is null;

Y¹ is selected from O (oxygen), S (sulfur), S(O), SO₂, and C(R²)₂ with the proviso that when X₁ is null Y₁ is C(R²)₂;

X² is C(R²)₂, or X² is null;

Y² is selected from O (oxygen), S (sulfur), S(O), SO₂, and C(R²)₂ with the proviso that when X² is null Y² is C(R²)₂;

each X³ is separately selected from the group consisting of NH, O (oxygen), and S (sulfur);

each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 5 halo, or optionally two vicinal R² and the carbons to which they are attached are together a fused three- to six-membered carbocyclic ring optionally substituted with up to two C₁₋₆alkyl groups;

each L₁ is separately selected from the group consisting of

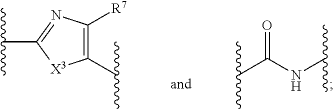

each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 5 halo and up to 5 hydroxy;

each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 5 halo; and

each R⁴ is separately selected from the group consisting of C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, C₁₋₆alkyl optionally substituted with up to 5 halo and up to 5 hydroxy, or optionally two geminal R⁴ are together oxo.

In some embodiments of Formula I,

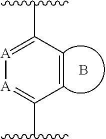

is selected from the group consisting of:

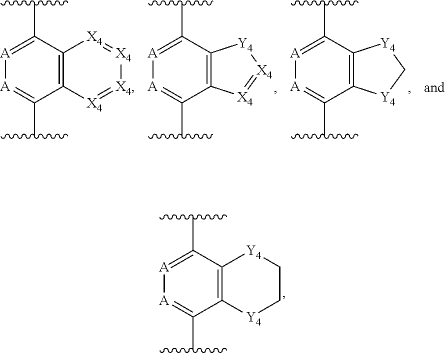

wherein,

each X₄ is separately selected from the group consisting of CR⁴ and N (nitrogen); and

each Y₄ is separately selected from the group consisting of C(R⁴)₂, NR⁴, O (oxygen), and S (sulfur).

In some embodiments of Formula I, each Z is null.

In some embodiments, the compound of Formula I has the structure of Formula Ia:

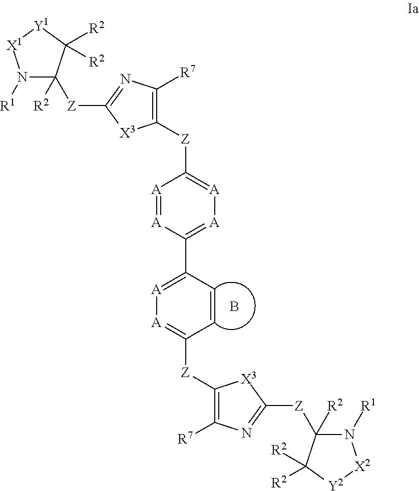

or a pharmaceutically acceptable salt thereof.

In some embodiments, the compound of Formula I has the structure of Formula Ib:

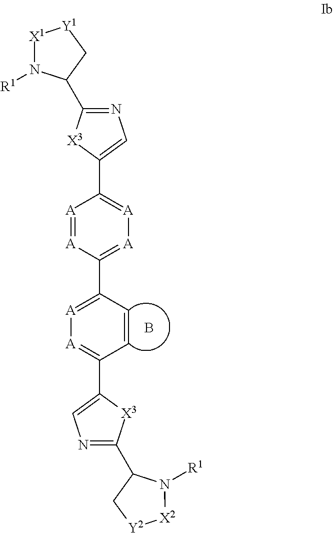

or a pharmaceutically acceptable salt thereof.

In some embodiments, the compound of Formula I has the structure of Formula Ic:

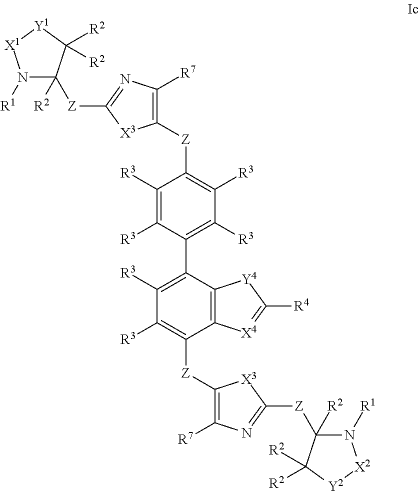

or a pharmaceutically acceptable salt thereof, wherein:

each X⁴ is separately selected from the group consisting of CH, CR⁴ and N (nitrogen); and

each Y⁴ is separately selected from the group consisting of CH₂, CHR⁴, C(R⁴)₂, NR⁴, O (oxygen), and S (sulfur).

In some embodiments, the compound of Formula I has the structure of Formula Id:

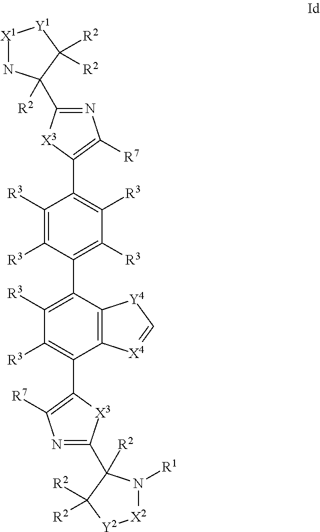

or a pharmaceutically acceptable salt thereof, wherein:

each X⁴ is separately selected from the group consisting of CH, CR⁴ and N (nitrogen); and

each Y⁴ is separately selected from the group consisting of CH₂, CHR⁴, C(R⁴)₂, NR⁴, O (oxygen), and S (sulfur).

In some embodiments, the compound of Formula I has the structure of Formula Ie:

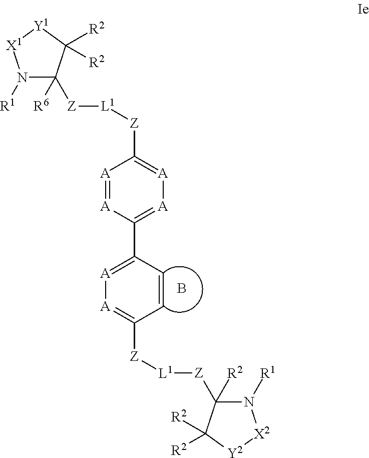

or a pharmaceutically acceptable salt thereof, wherein:

R⁶ is C₁₋₆alkyl optionally substituted with up to 9 halo.

In some embodiments, the compound of Formula I has the structure of Formula If:

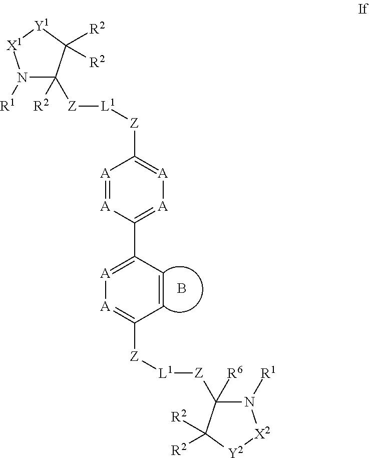

or a pharmaceutically acceptable salt thereof, wherein:

R⁶ is C₁₋₆alkyl optionally substituted with up to 9 halo.

In some embodiments of Formula I, Formula Ia, Formula Ib, Formula Ic, Formula Id, Formula Ie, or Formula If, each R¹ is R^{1a}C(═O)—.

In some embodiments of Formula I, Formula Ia, Formula Ib, Formula Ic, Formula Id, Formula Ie, or Formula If, each R^{1a} is —CHR^{2a}NHR^{3b}.

In some embodiments of Formula I, Formula Ia, Formula Ib, Formula Ic, Formula Id, Formula Ie, or Formula If, each R^{2a} is C₁₋₆alkyl; each R^{3b} is —C(═O)OR⁵; and each R⁵ is C₁₋₆alkyl.

In some embodiments, the compound of Formula I has the structure

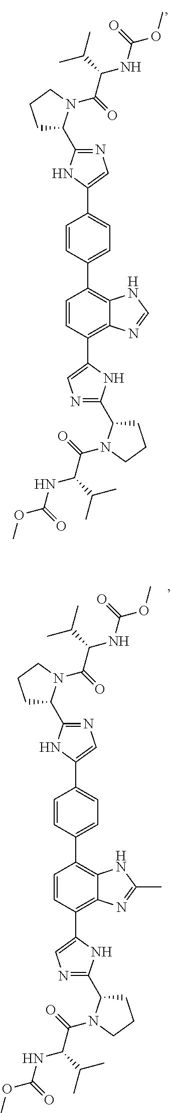

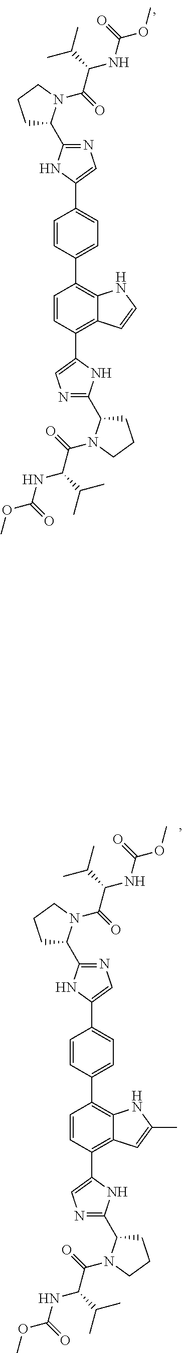

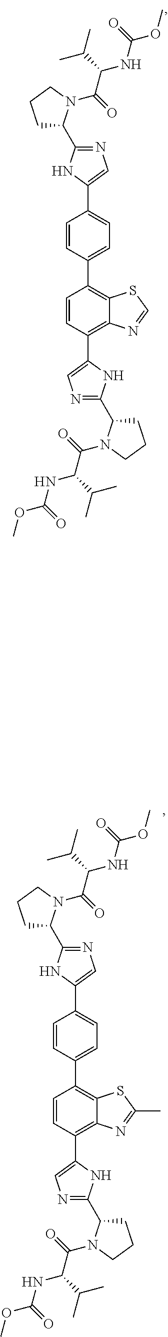

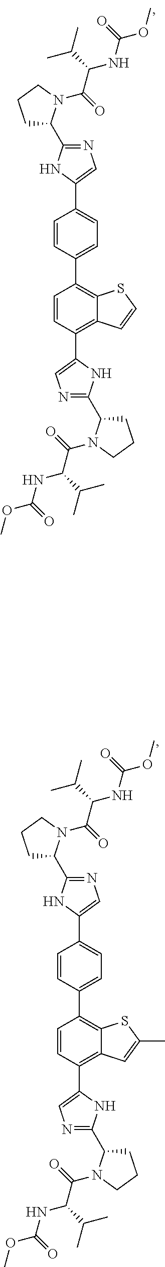

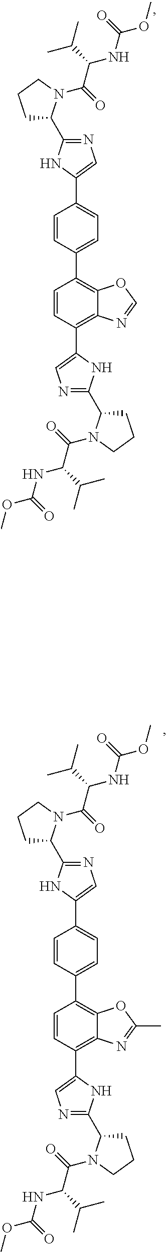

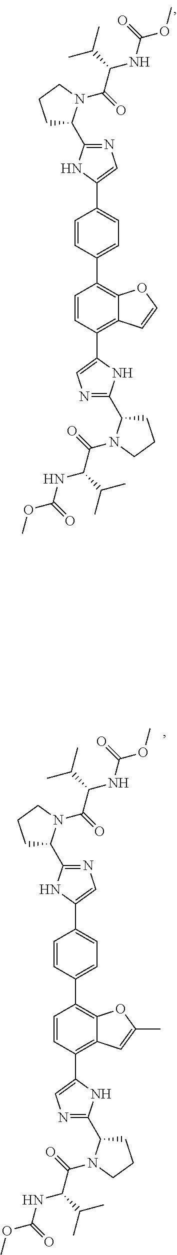

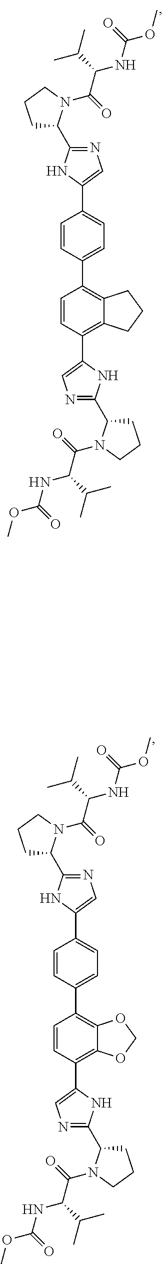

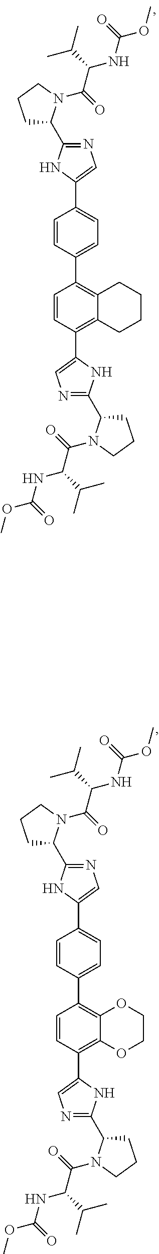

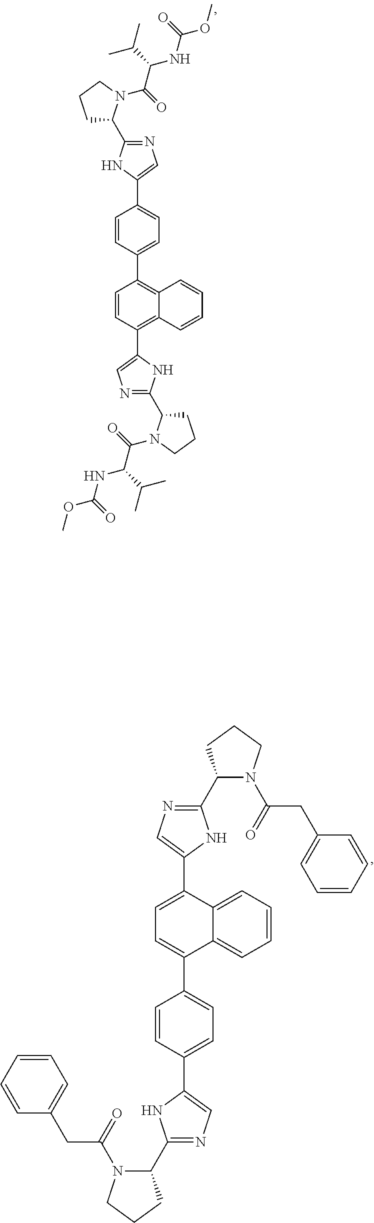

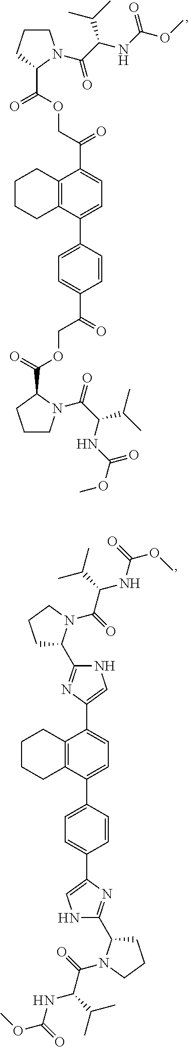

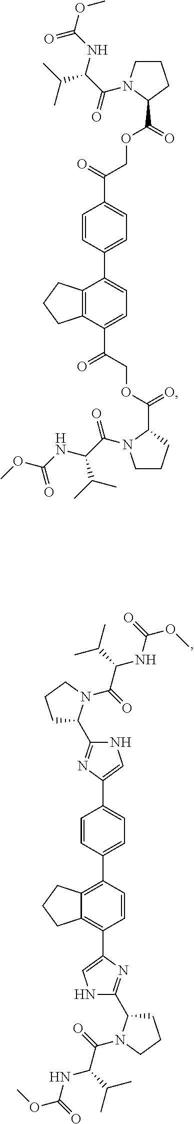

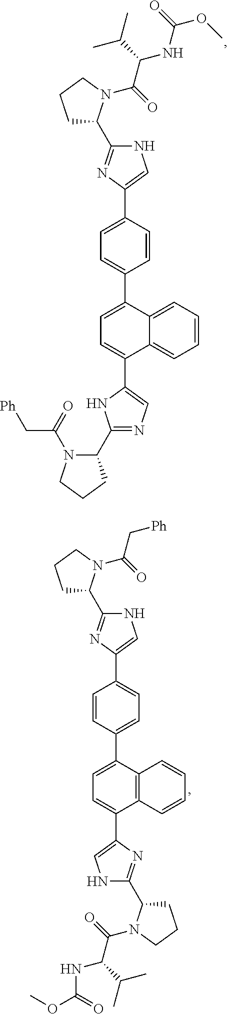

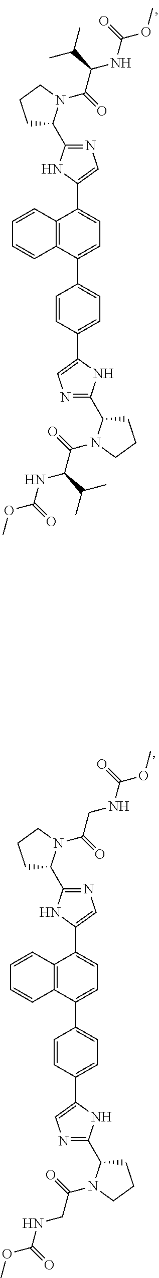

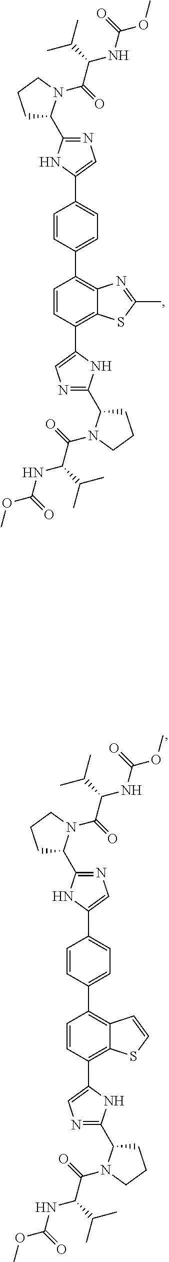

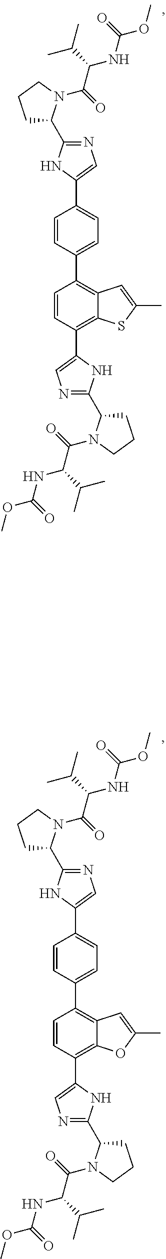

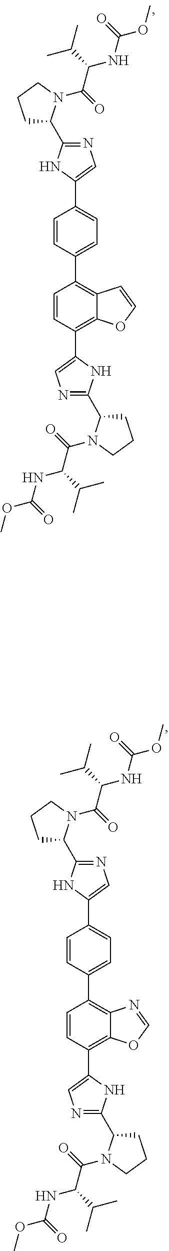

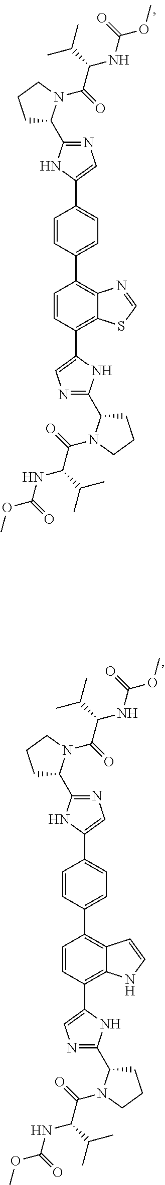

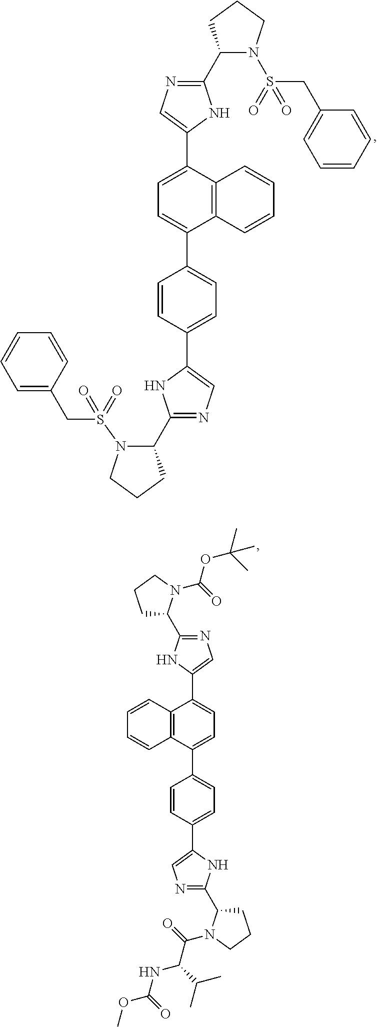

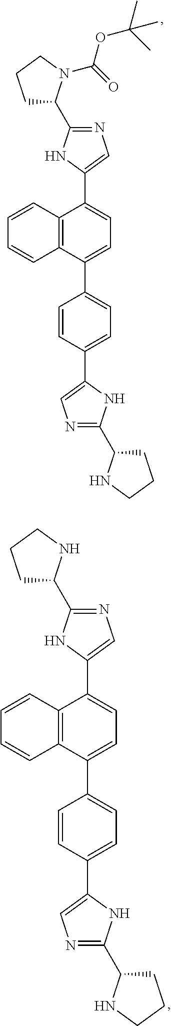

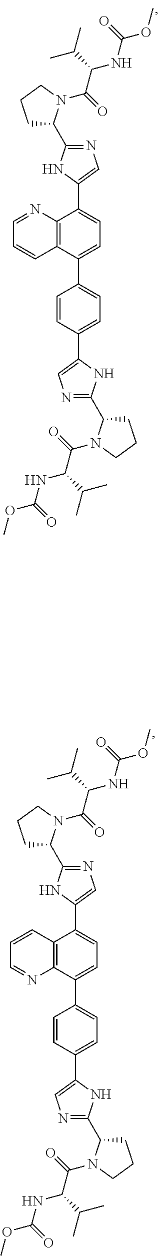

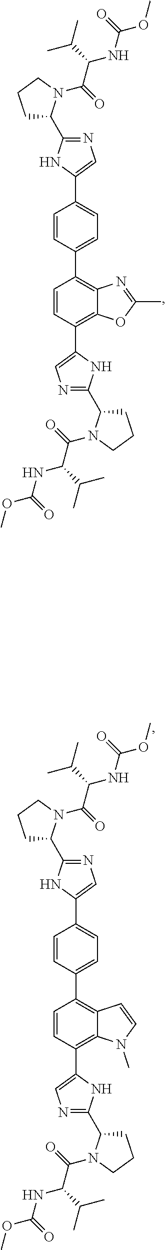

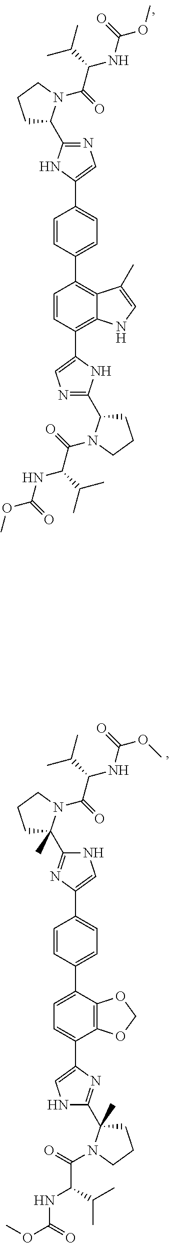

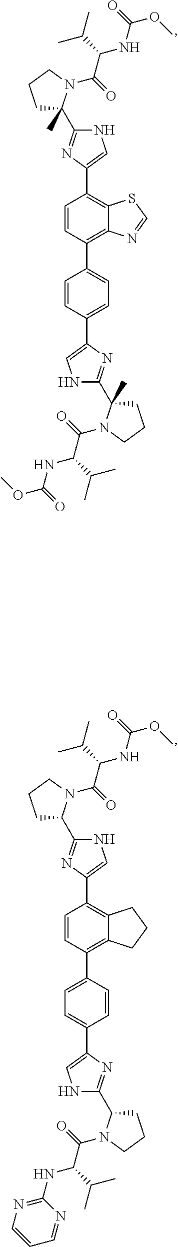

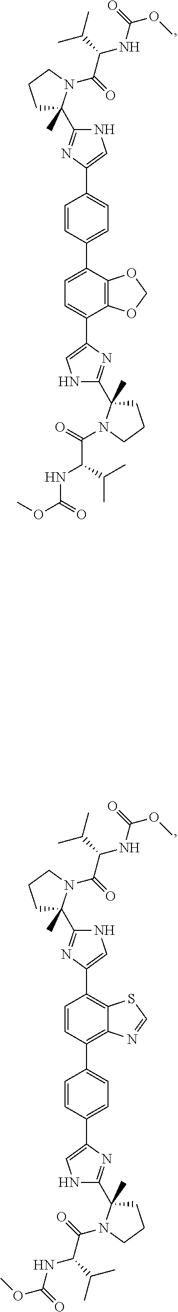

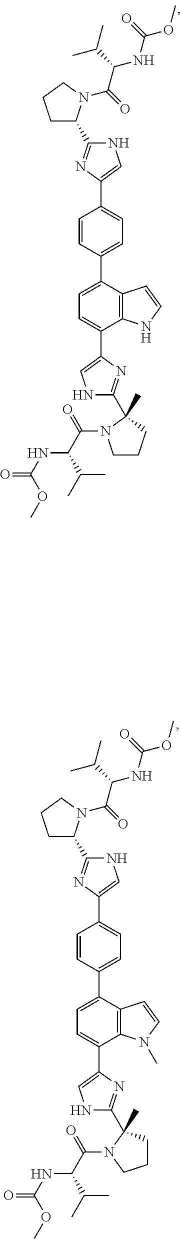

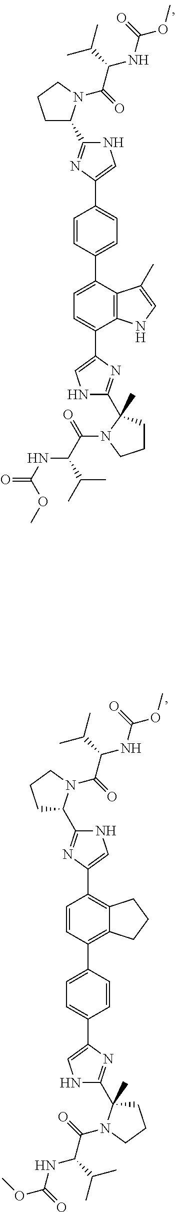

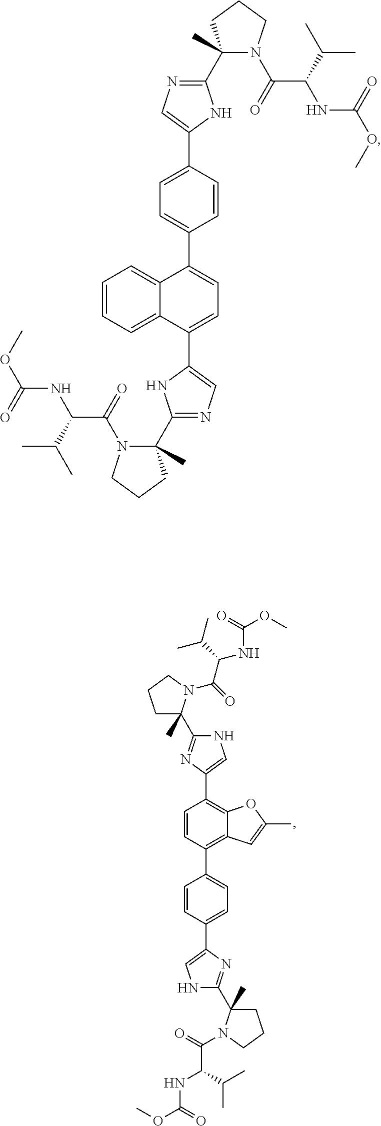

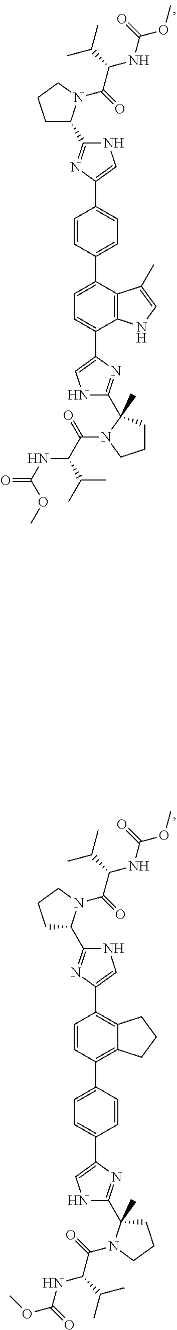

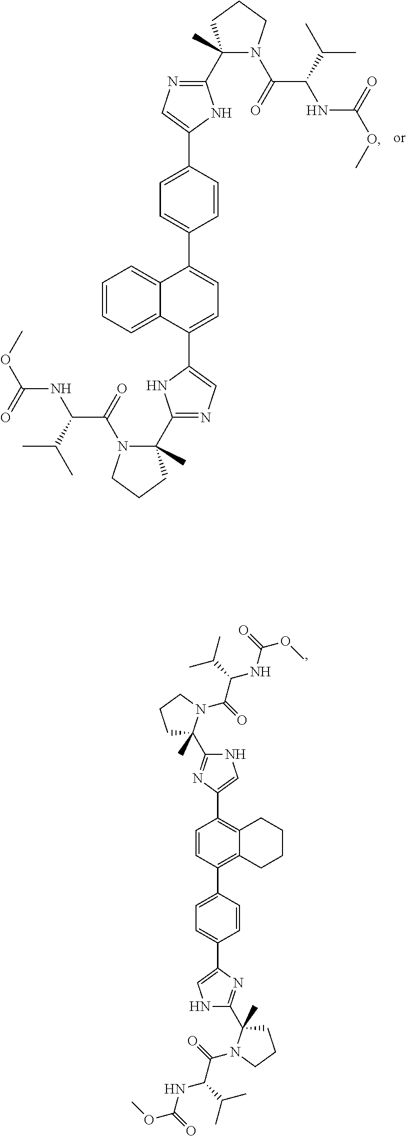

or a pharmaceutically acceptable salt thereof.

In some embodiments of Formula I, the compound does not have the structure:

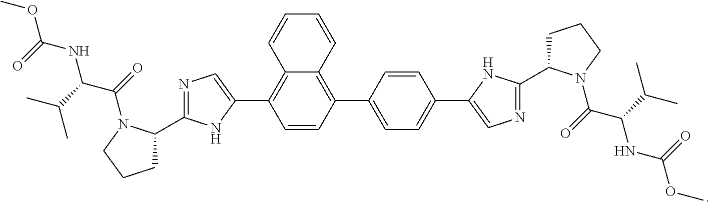

Additional embodiments include a compound having the structure of Formula II:

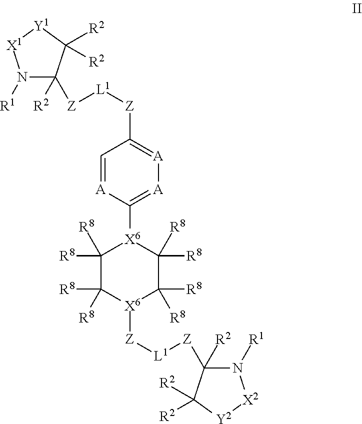

or a pharmaceutically acceptable salt thereof,

wherein:

each R¹ is separately selected from the group consisting of hydrogen and R^{1a}C(═O)— and R^{1a}C(═S)—;

each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;

each R^{c}R^{d}N is separately selected, wherein R^{c} and R^{d} are each independently selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;

each R^{e}R^{f}N is separately selected, wherein R^{e} and R^{f} are each separately selected from the group consisting of hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O);

each R^{x}R^{y}N is separately selected, wherein R^{x} and R^{y} are each separately selected from the group consisting of hydrogen, alkylOC(═O)—, alkyl, alkylC(═O)—, aryl, arylalkyl, cycloalkyl, and heterocyclyl;

each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is

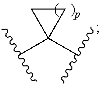

each R^{3a} is separately selected from the group consisting of hydrogen, and optionally substituted C₁₋₆alkyl;

each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;

each R^{4a}R^{4b}N is separately selected, wherein R^{4a} and R^{4b} are each separately selected from the group consisting of hydrogen, optionally substituted C₁₋₆alkyl, and aryl(CH₂)ₙ—;

each R^{5a} is separately selected from the group consisting of optionally substituted C₁₋₆alkyl, and aryl(CH₂)ₙ—;

each R^{6a} is separately selected from the group consisting of optionally substituted C₁₋₆alkyl, and aryl(CH₂)ₙ—;

X¹ is (C(R²)₂)_{q},

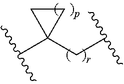

or X¹ is null;

Y¹ is selected from O (oxygen), S (sulfur), S(O), SO₂, NR², and C(R²)₂ with the proviso that when X¹ is null Y¹ is C(R²)₂;

X² is (C(R²)₂)_{q},

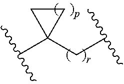

or X² is null;

Y² is selected from O (oxygen), S (sulfur), S(O), SO₂, NR², and C(R²)₂ with the proviso that when X² is null Y² is C(R²)₂;

each X⁶ is separately selected from the group consisting of N (nitrogen), and CR⁸;

each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl group;

each R^{a}R^{b}N is separately selected, wherein R^{a} and R^{b} are each separately selected from the group consisting of hydrogen, C₂₋₆alkenyl, and C₁₋₆alkyl;

each Z is separately selected, wherein Z is selected from the group consisting of O (oxygen) and CH₂, or Z is null;

each A is separately selected from the group consisting of CR³ and N (nitrogen);

each L¹ is separately selected from the group consisting of

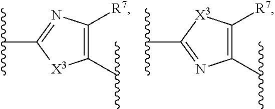

—C(═O)(CH₂)ₘOC(═O)—, —C(CF₃)₂NR^{2c}—, and

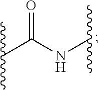

each X³ is separately selected from the group consisting of NH, NC₁₋₆alkyl, O (oxygen), and S (sulfur);

each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 9 halo and up to 5 hydroxy;

each m separately is 1 or 2;

each n separately is 0, 1 or 2;

each p separately is 1, 2, 3 or 4;

each q separately is 1, 2, 3, 4 or 5;

each r separately is 0, 1, 2, 3, or 4;

each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo; and

each R⁸ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 9 halo and up to 5 hydroxy, or optionally two geminal R⁸ are together oxo.

In some embodiments of Formula II, each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, (R^{c}R^{d}N)(CH═CH)ₘ—, (R^{c}R^{d}N)alkyl, (R^{c}R^{d}N)C(═O)—, C₁₋₆alkoxy optionally substituted with up to 5 halo, and C₁₋₆alkyl optionally substituted with up to 5 halo;

each R^{c}R^{d}N is separately selected, wherein R^{c} and R^{d} are each independently selected from the group consisting of hydrogen, alkoxyC(═O)—, C₁₋₆alkyl, C₁₋₆alkylC(═O)—, C₁₋₆alkylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 5 halo, and C₁₋₆alkyl optionally substituted with up to 5 halo;

each R^{2a} is separately selected from the group consisting of hydrogen, C₁₋₆alkyl, aryl(CH₂)ₙ—, and heteroaryl(CH₂)ₙ—;

each R^{3a} is separately selected from the group consisting of hydrogen, and C₁₋₆alkyl;

each R^{3b} is separately selected from the group consisting of C₁₋₆alkyl, —(CH₂)ₙC(═O)NR^{4a}R^{4b}, —(CH₂)ₙC(═O)OR^{5a}, and —(CH₂)ₙC(═O)R^{6a};

each R^{4a}R^{4b}N is separately selected, wherein R^{4a} and R^{4b} are each separately selected from the group consisting of hydrogen, C₁₋₆alkyl, and aryl(CH₂)ₙ—;

each R^{5a} is separately selected from the group consisting of C₁₋₆alkyl, and aryl(CH₂)ₙ—;

each R^{6a} is separately selected from the group consisting of C₁₋₆alkyl, and aryl(CH₂)ₙ—;

X¹ is C(R²)₂, or X¹ is null;

Y¹ is selected from O (oxygen), S (sulfur), S(O), SO₂, and C(R²)₂ with the proviso that when X¹ is null Y¹ is C(R²)₂;

X² is C(R²)₂, or X² is null;

Y² is selected from O (oxygen), S (sulfur), S(O), SO₂, and C(R²)₂ with the proviso that when X² is null Y² is C(R²)₂;

each X³ is separately selected from the group consisting of NH, O (oxygen), and S (sulfur);

each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 5 halo, or optionally two vicinal R² and the carbons to which they are attached are together a fused three- to six-membered carbocyclic ring optionally substituted with up to two C₁₋₆alkyl group;

each L¹ is separately selected from the group consisting of

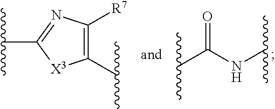

each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 5 halo and up to 5 hydroxy;

each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 5 halo; and

each R⁸ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 5 halo and up to 5 hydroxy, or optionally two geminal R⁸ are together oxo.

In some embodiments, the compound of Formula II has the structure of Formula IIa:

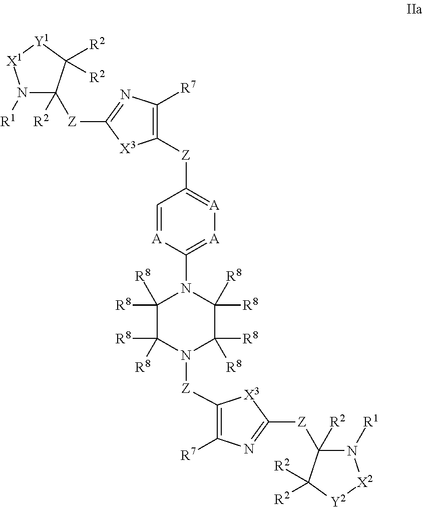

or a pharmaceutically acceptable salt thereof.

In some embodiments of Formula II, or Formula IIa, each Z is null.

In some embodiments, the compound of Formula II has the structure of Formula IIb:

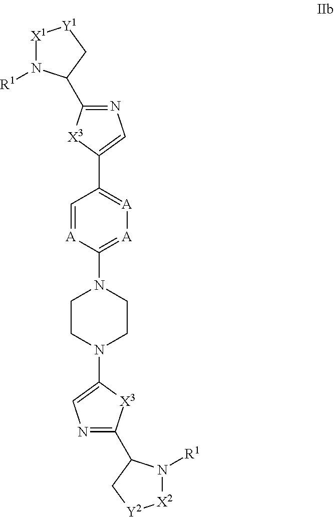

or a pharmaceutically acceptable salt thereof.

In some embodiments of Formula II, Formula IIa, or Formula IIb, each R¹ is R^{1a}C(═O)—.

In some embodiments of Formula II, Formula IIa, or Formula IIb, each R^{1a} is —CHR^{2a}NHR^{3b}.

In some embodiments of Formula II, Formula IIa, or Formula IIb, each R^{2a} is C₁₋₆alkyl; each R^{3b} is —C(═O)OR⁵; and each R⁵ is C₁₋₆alkyl.

In some embodiments, the compound of Formula II has the structure

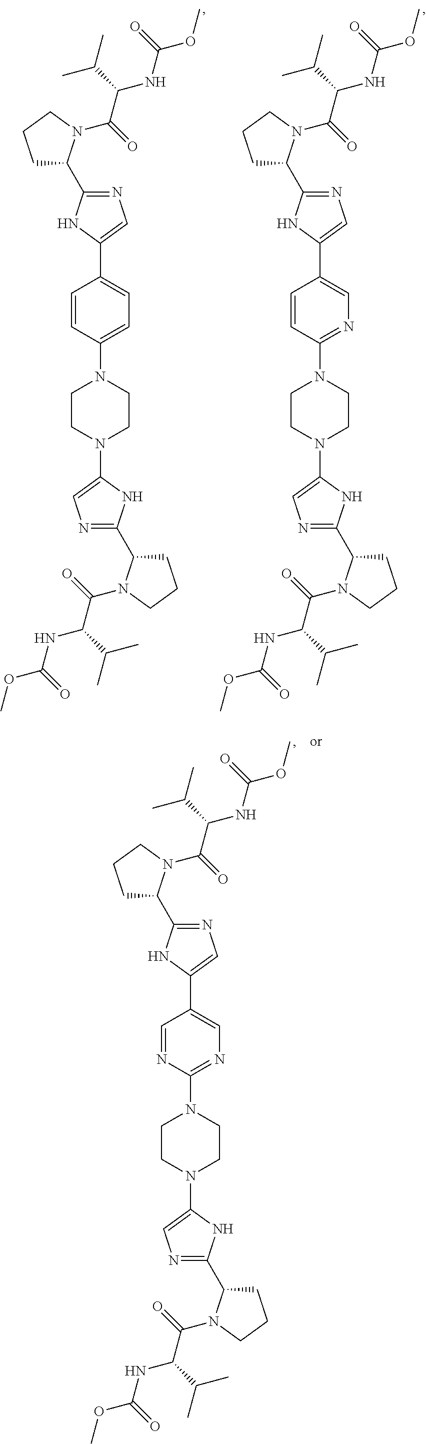

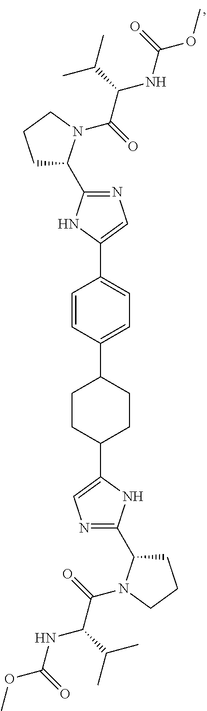

or a pharmaceutically acceptable salt thereof.

In some embodiments of Formula II, at least one A is N (nitrogen) or both X⁶ are N (nitrogen).

In some embodiments of Formula II, the compound is not selected from the group consisting of:

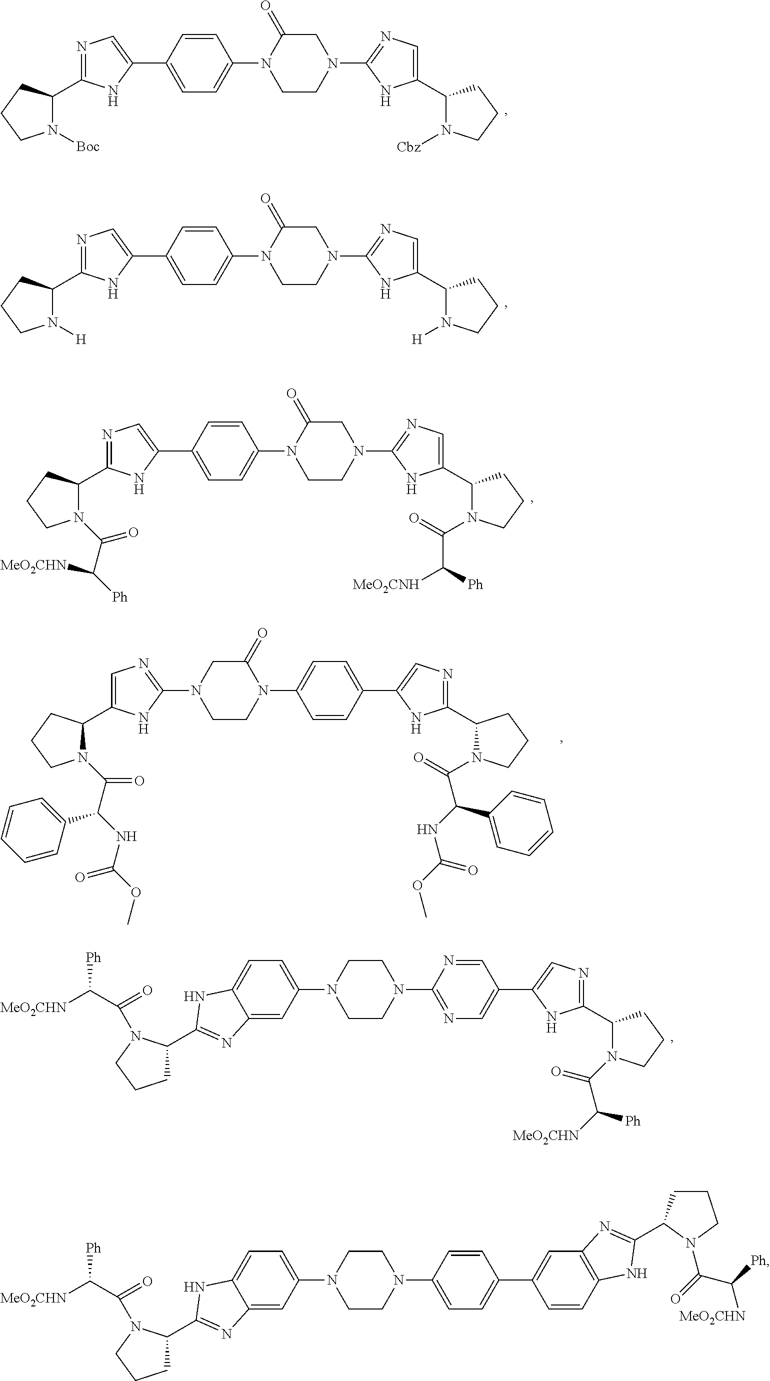

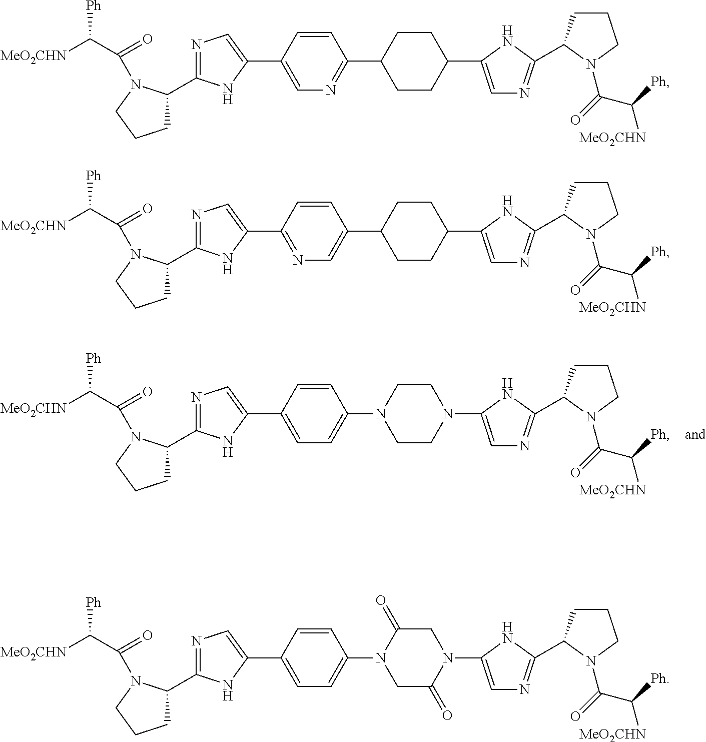

Additional embodiments include a compound having the structure of Formula III:

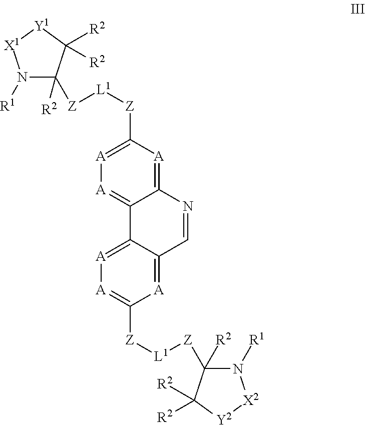

or a pharmaceutically acceptable salt thereof,

wherein:

each R¹ is separately selected from the group consisting of hydrogen and R^{1a}C(═O)— and R^{1a}C(═S)—;

each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;

each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;

each R^{e}R^{f}N is separately selected, wherein R^{e} and R^{f} are each separately selected from the group consisting of hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;

each R^{x}R^{y}N is separately selected, wherein R^{x} and R^{y} are each separately selected from the group consisting of hydrogen, C₁₋₆alkylOC(═O)—, alkyl, alkylC(═O)—, aryl, arylalkyl, cycloalkyl, and heterocyclyl;

each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl, and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is

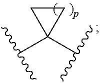

each R^{3a} is separately selected from the group consisting of hydrogen, and optionally substituted C₁₋₆alkyl;

each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;

each R^{4a}R^{4b}N is separately selected, wherein R^{4a} and R^{4b} are each separately selected from the group consisting of hydrogen, optionally substituted C₁₋₆alkyl, and aryl(CH₂)ₙ—;

each R^{5a} is separately selected from the group consisting of optionally substituted C₁₋₆alkyl, and aryl(CH₂)ₙ—;

each R^{6a} is separately selected from the group consisting of optionally substituted C₁₋₆alkyl, and aryl(CH₂)ₙ—;

X¹ is (C(R²)₂)_{q},

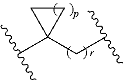

or X¹ is null;

Y¹ is selected from O (oxygen), S (sulfur), S(O), SO₂, NR², and C(R²)₂ with the proviso that when X¹ is null Y¹ is C(R²)₂;

X² is (C(R²)₂)_{q},

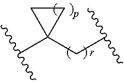

or X² is null;

Y² is selected from O (oxygen), S (sulfur), S(O), SO₂, NR², and C(R²)₂ with the proviso that when X² is null Y² is C(R²)₂;

each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups;

each R^{a}R^{b}N is separately selected, wherein R^{a} and R^{b} are each separately selected from the group consisting of hydrogen, C₂₋₆alkenyl, and C₁₋₆alkyl;

each Z is separately selected, wherein Z is selected from the group consisting of O (oxygen) and CH₂, or Z is null;

each A is separately selected from the group consisting of CR³ and N (nitrogen);

each L¹ is separately selected from the group consisting of

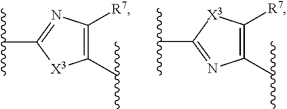

—C(═O)(CH₂)ₘOC(═O)—, —C(CF₃)₂NR^{2c}, and

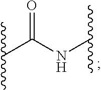

each X³ is separately selected from the group consisting of NH, NC₁₋₆alkyl, O (oxygen), and S (sulfur);

each m separately is 1 or 2;

each n separately is 0, 1 or 2;

each p separately is 1, 2, 3 or 4;

each q separately is 1, 2, 3, 4 or 5;

each r separately is 0, 1, 2, 3, or 4;

each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 9 halo and up to 5 hydroxy; and

each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo.

In some embodiments of Formula III, each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH), heterocyclylalkoxy, heterocyclylalkyl, heterocyclylOalkyl, hydroxyalkyl, R^{c}R^{d}N—, (R^{c}R^{d}N)(CH═CH)ₘ—, (R^{c}R^{d}N)alkyl, (R^{c}R^{d}N)C(═O)—, C₁₋₆alkoxy optionally substituted with up to 5 halo, and C₁₋₆alkyl optionally substituted with up to 5 halo;

each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 5 halo, and C₁₋₆alkyl optionally substituted with up to 5 halo;

each R^{2a} is separately selected from the group consisting of hydrogen, C₁₋₆alkyl, aryl(CH₂)ₙ—, and heteroaryl(CH₂)ₙ—;

each R^{3a} is separately selected from the group consisting of hydrogen, and C₁₋₆alkyl;

each R^{3b} is separately selected from the group consisting of C₁₋₆alkyl, —(CH₂)ₙC(═O)NR^{4a}R^{4b}, —(CH₂)ₙC(═O)OR^{5a}, and —(CH₂)ₙC(═O)R^{6a};

each R^{4a}R^{4b}N is separately selected, wherein R^{4a} and R^{4b} are each separately selected from the group consisting of hydrogen, C₁₋₆alkyl, and aryl(CH₂)ₙ—;

each R^{5a} is separately selected from the group consisting of optionally substituted C₁₋₆alkyl, and aryl(CH₂)ₙ—;

each R^{6a} is separately selected from the group consisting of optionally substituted C₁₋₆alkyl, and aryl(CH₂)ₙ—;

X¹ is C(R²)₂, or X¹ is null;

Y¹ is selected from O (oxygen), S (sulfur), S(O), SO₂, and C(R²)₂ with the proviso that when X¹ is null Y¹ is C(R²)₂;

X² is C(R²)₂, or X² is null;

Y² is selected from O (oxygen), S (sulfur), S(O), SO₂, and C(R²)₂ with the proviso that when X² is null Y² is C(R²)₂;

each X³ is separately selected from the group consisting of NH, O (oxygen), and S (sulfur);

each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 5 halo, or optionally two vicinal R² and the carbons to which they are attached are together a fused three- to six-membered carbocyclic ring optionally substituted with up to two C₁₋₆alkyl groups;

each L¹ is separately selected from the group consisting of

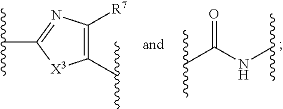

each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 5 halo and up to 5 hydroxy; and

each R⁷ is separately selected from the group consisting of hydrogen, C₁₋₆alkylOC(═O)—, arylalkylOC(═O)—, —COOH, (R^{a}R^{b}N)C(═O)—, tri alkyl silylalkylOalkyl, and C₁₋₆alkyl optionally substituted with up to 5 halo.

In some embodiments, the compound of Formula III has the structure of Formula IIIa:

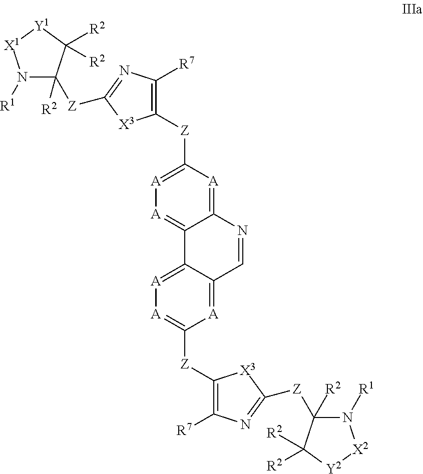

or a pharmaceutically acceptable salt thereof.

In some embodiments of Formula III or IIIa, each Z is null.

In some embodiments, the compound of Formula III has the structure of Formula IIIb:

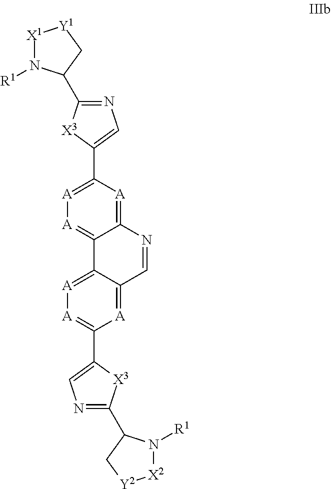

or a pharmaceutically acceptable salt thereof.

In some embodiments of Formula III, Formula IIIa, or Formula IIIb, each R¹ is R^{1a}C(═O)—.

In some embodiments of Formula III, Formula IIIa, or Formula IIIb, each R^{1a} is —CHR^{2a}NHR^{3b}.

In some embodiments of Formula III, Formula IIIa, or Formula IIIb, each R^{2a} is C₁₋₆alkyl; each R^{3b} is —C(═O)OR⁵; and each R⁵ is C₁₋₆alkyl.

In some embodiments, the compound of Formula III has the structure

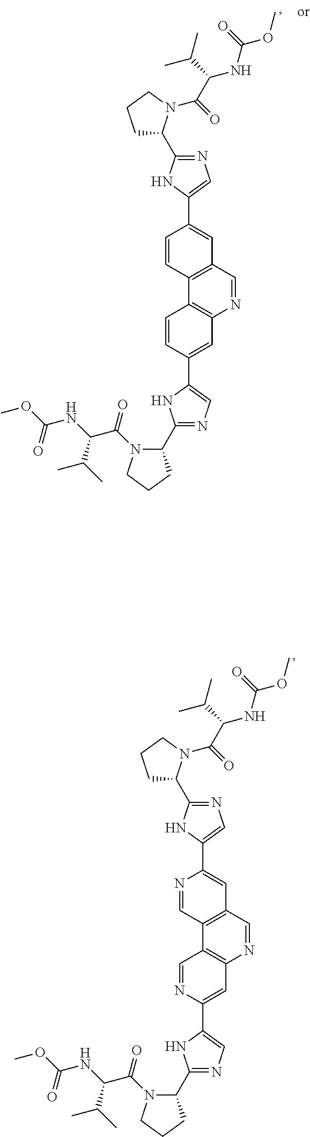

or a pharmaceutically acceptable salt thereof.

Additional embodiments include a compound having the structure of Formula IV:

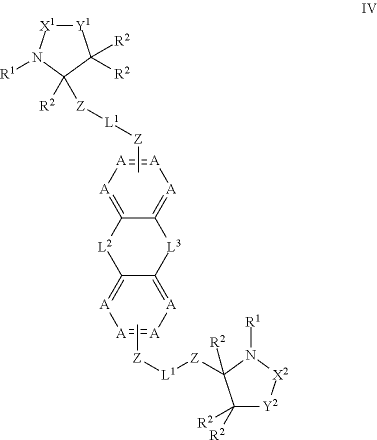

or a pharmaceutically acceptable salt thereof,

wherein:

each R¹ is separately selected from the group consisting of hydrogen and R^{1a}C(═O)— and R^{1a}C(═S)—;

each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;

each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;

each R^{e}R^{f}N is separately selected, wherein R^{e} and R^{f} are each separately selected from the group consisting of hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;

each R^{x}R^{y}N is separately selected, wherein R^{x} and R^{y} are each separately selected from the group consisting of hydrogen, C₁₋₆alkylOC(═O)—, C₁₋₆alkyl, C₁₋₆alkylC(═O)—, aryl, arylalkyl, cycloalkyl, and heterocyclyl;

each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is

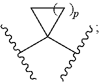

each R^{3a} is separately selected from the group consisting of hydrogen, and optionally substituted C₁₋₆alkyl;

each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;

each R^{4a}R^{4b}N is separately selected, wherein R^{4a} and R^{4b} are each separately selected from the group consisting of hydrogen, optionally substituted C₁₋₆alkyl, and aryl(CH₂)ₙ—;

each R^{5a} is separately selected from the group consisting of optionally substituted C₁₋₆alkyl, and aryl(CH₂)ₙ—;

each R^{6a} is separately selected from the group consisting of optionally substituted C₁₋₆alkyl, and aryl(CH₂)ₙ—;

X¹ is (C(R²)₂)_{q},

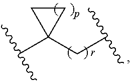

or X¹ is null;

Y¹ is selected from O (oxygen), S (sulfur), S(O), SO₂, NR², and C(R²)₂ with the proviso that when X¹ is null Y¹ is C(R²)₂;

X² is (C(R²)₂)_{q},

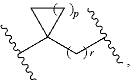

or X² is null;

Y² is selected from O (oxygen), S (sulfur), S(O), SO₂, NR², and C(R²)₂ with the proviso that when X² is null Y² is C(R²)₂;

each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups;

each R^{a}R^{b}N is separately selected, wherein R^{a} and R^{b} are each separately selected from the group consisting of hydrogen, C₂₋₆alkenyl, and C₁₋₆alkyl;

each Z is separately selected, wherein Z is selected from the group consisting of O (oxygen) and CH₂, or Z is null;

each A is separately selected from the group consisting of CR³ and N (nitrogen);

each L¹ is separately selected from the group consisting of

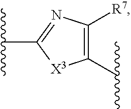

—C(═O)(CH₂)ₘOC(═O)—, —C(CF₃)₂NR^{2c}—, and

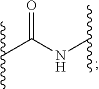

each X₃ is separately selected from the group consisting of NH, NC₁₋₆alkyl, O (oxygen), and S (sulfur);

L² is selected from the group consisting of —C(═O)—, —(CH₂CH₂)—, —(CH₂O)—, —(CH₂S)—, —(CH═CH)—, —(CH═N)—, —NH—, O (oxygen), S (sulfur), and —CH₂—;

L³ is selected from the group consisting of

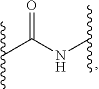

—(NR⁹)—, O (oxygen), S (sulfur), and —CH₂—;

R⁹ is selected from the group consisting of hydrogen and —C(═O)R^{9a};

R^{9a} is selected from the group consisting of —NR^{9b}R^{9c}, —OR^{9d}, C₁₋₆alkoxy optionally substituted with up to 9 halo, C₁₋₆alkyl optionally substituted with up to 9 halo, and optionally substituted aryl;

R^{9b} is selected from the group consisting of hydrogen, C₁₋₆alkyl optionally substituted with up to 9 halo, and optionally substituted aryl;

R^{9c} is selected from the group consisting of C₁₋₆alkyl optionally substituted with up to 9 halo, and optionally substituted aryl;

R^{9d} is selected from the group consisting of C₁₋₆alkyl optionally substituted with up to 9 halo, and optionally substituted aryl;

each m separately is 1 or 2;

each n separately is 0, 1 or 2;

each p separately is 1, 2, 3 or 4;

each q separately is 1, 2, 3, 4 or 5;

each r separately is 0, 1, 2, 3, or 4;

each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 9 halo and up to 5 hydroxy; and

each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo.

In some embodiments of Formula III, each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, (R^{c}R^{d}N)(CH═CH)ₘ—, (R^{c}R^{d}N)alkyl, (R^{c}R^{d}N)C(═O)—, C₁₋₆alkoxy optionally substituted with up to 5 halo, and C₁₋₆alkyl optionally substituted with up to 5 halo;

each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl; (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 5 halo, and C₁₋₆alkyl optionally substituted with up to 5 halo;

each R^{2a} is separately selected from the group consisting of hydrogen, C₁₋₆alkyl, aryl(CH₂)ₙ—, and heteroaryl(CH₂)ₙ—;

each R^{3a} is separately selected from the group consisting of hydrogen, and C₁₋₆alkyl;

each R^{3b} is separately selected from the group consisting of C₁₋₆alkyl, —(CH₂)ₙC(═O)NR^{4a}R^{4b}, —(CH₂)ₙC(═O)OR^{5a}, and —(CH₂)ₙC(═O)R^{6a};

each R^{4a}R^{4b}N is separately selected, wherein R^{4a} and R^{4b} are each separately selected from the group consisting of hydrogen, C₁₋₆alkyl, and aryl(CH₂)ₙ—;

each R^{5a} is separately selected from the group consisting of C₁₋₆alkyl, and aryl(CH₂)ₙ—;

each R^{6a} is separately selected from the group consisting of C₁₋₆alkyl, and aryl(CH₂)ₙ—;

X¹ is C(R²)₂, or X¹ is null;

Y¹ is selected from O (oxygen), S (sulfur), S(O), SO₂, and C(R²)₂ with the proviso that when X¹ is null Y¹ is C(R²)₂;

X² is C(R²)₂, or X² is null;

Y² is selected from (oxygen), S (sulfur), S(O), SO₂, and C(R²)₂ with the proviso that when X² is null Y² is C(R²)₂;

each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 5 halo, or optionally two vicinal R² and the carbons to which they are attached are together a fused three- to six-membered carbocyclic ring optionally substituted with up to two C₁₋₆alkyl groups;

each L¹ is separately selected from the group consisting of

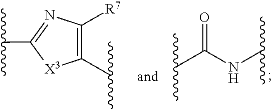

R^{9a} is selected from the group consisting of —NR^{9b}R^{9c}, —OR^{9d}, C₁₋₆alkyl optionally substituted with up to 5 halo, and optionally substituted aryl;

R^{9b} is selected from the group consisting of hydrogen, C₁₋₆alkyl optionally substituted with up to 5 halo, and optionally substituted aryl;

R^{9c} is selected from the group consisting of C₁₋₆alkyl optionally substituted with up to 5 halo, and optionally substituted aryl;

R^{9d} is selected from the group consisting of C₁₋₆alkyl optionally substituted with up to 5 halo, and optionally substituted aryl;

each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 5 halo and up to 5 hydroxy; and

each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 5 halo.

In some embodiments of Formula IV, each Z is null.

In some embodiments, the compound of Formula IV has the structure of one of the following formulas,

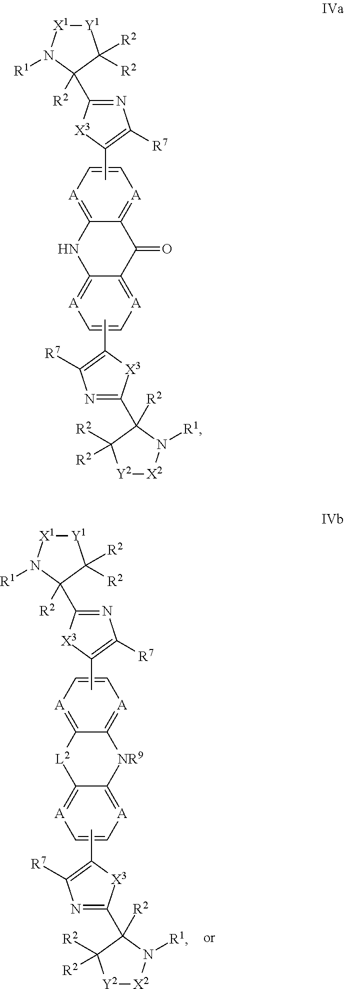

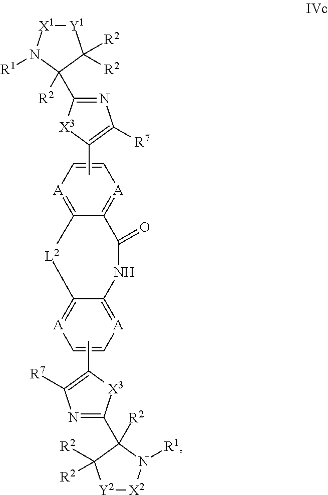

or pharmaceutically acceptable salts thereof.

In some embodiments of Formula IV, Formula IVa, Formula IVb, or Formula IVc, each R¹ is R^{1a}C(═O)—.

In some embodiments of Formula IV, Formula IVa, Formula IVb, or Formula IVc, each R^{1a} is —CHR^{2a}NHR^{3b}.

In some embodiments of Formula IV, Formula IVa, Formula IVb, or Formula IVc, each R^{2a} is C₁₋₆alkyl; each R^{3b} is —C(═O)OR⁵; and each R⁵ is C₁₋₆alkyl.

In some embodiments, the compound of Formula IV has the structure

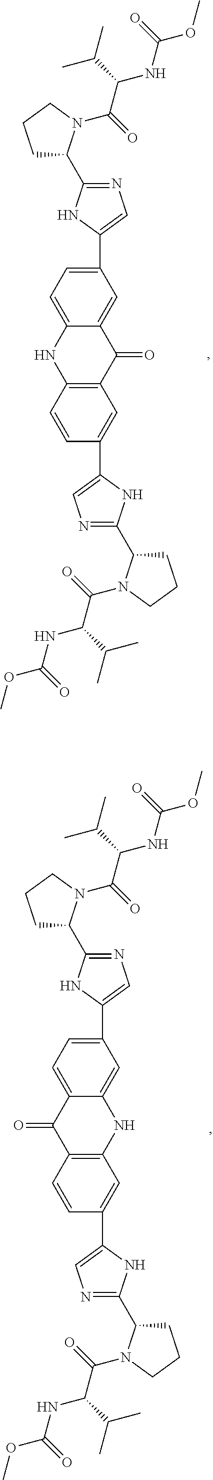

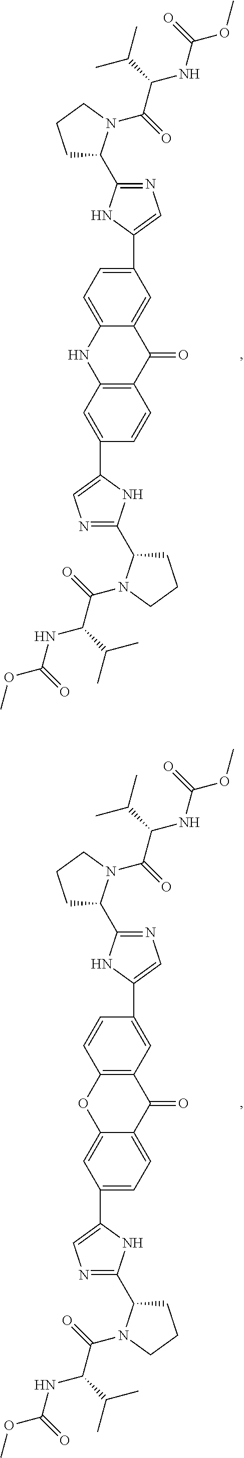

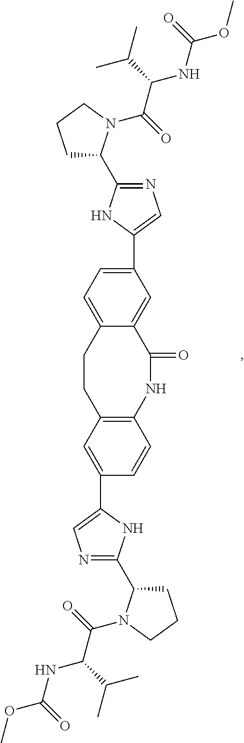

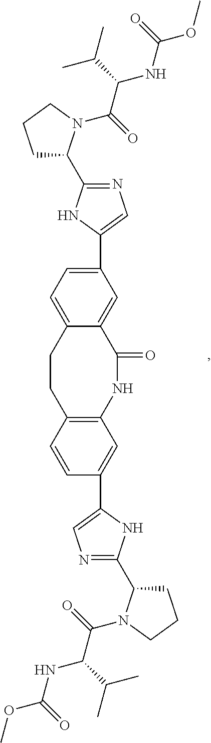

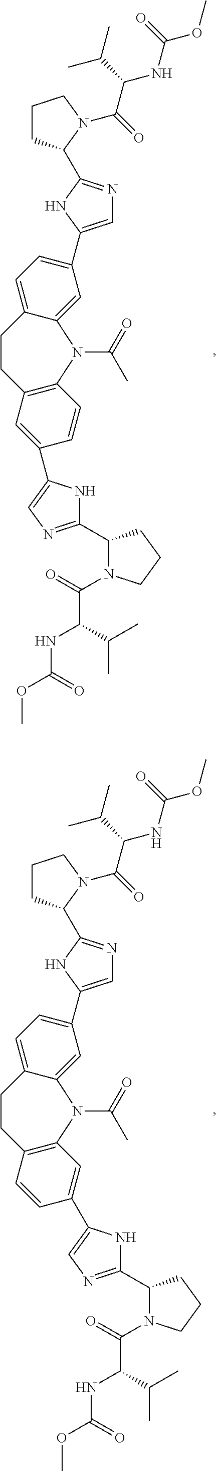

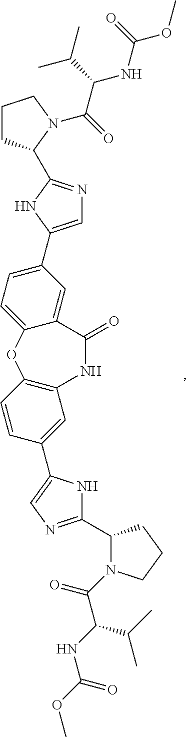

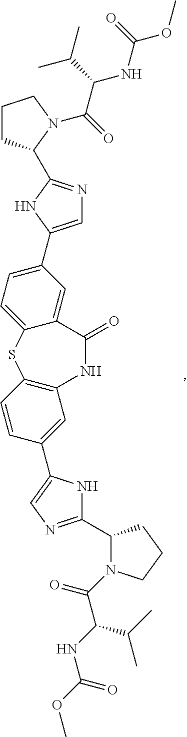

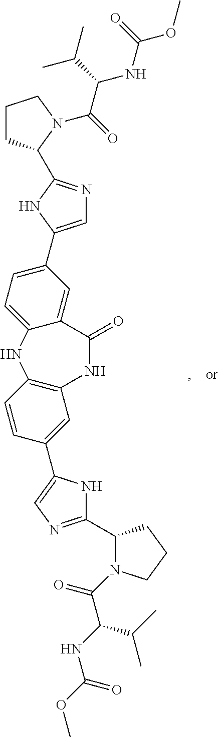

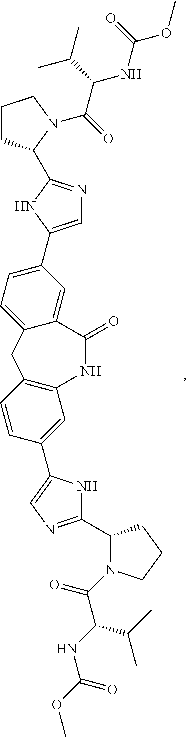

or a pharmaceutically acceptable salt thereof.

In some embodiments of Formula IV, the compound is not selected from the group consisting of:

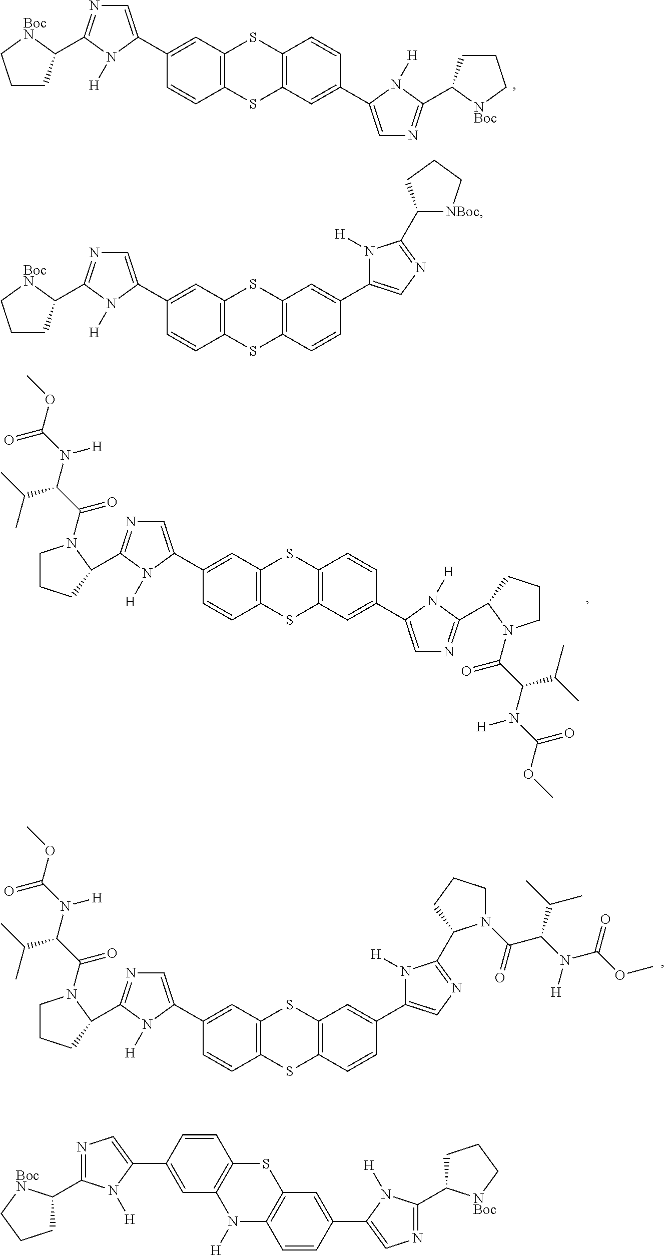

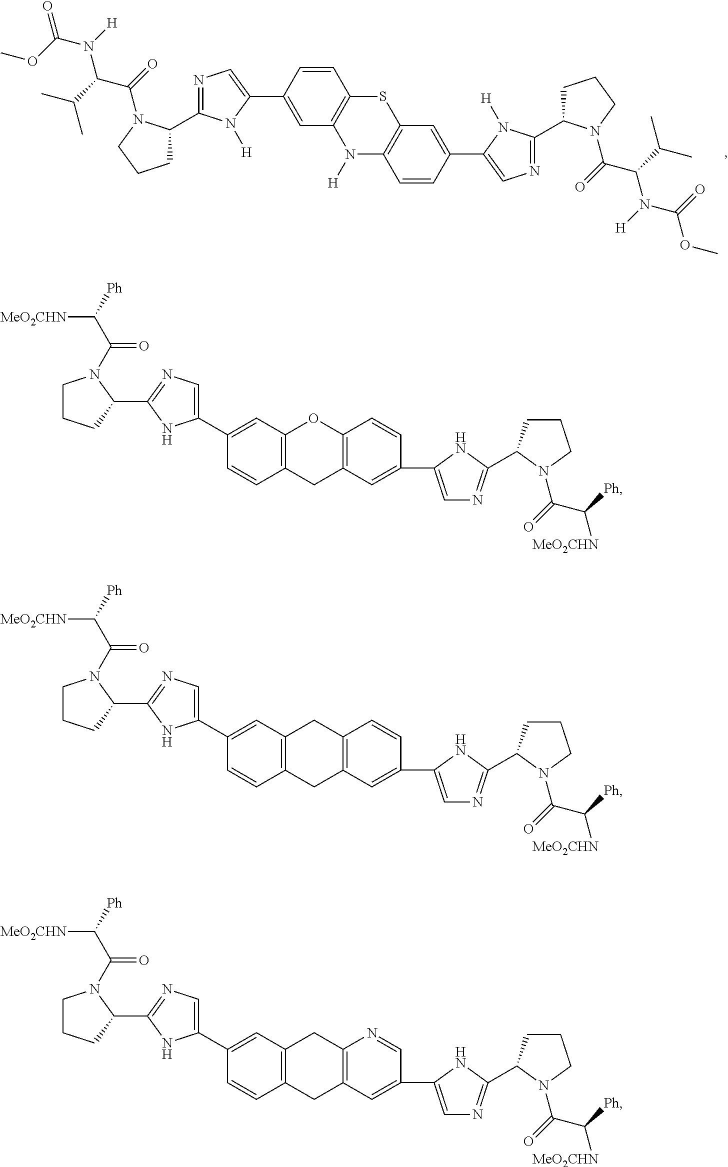

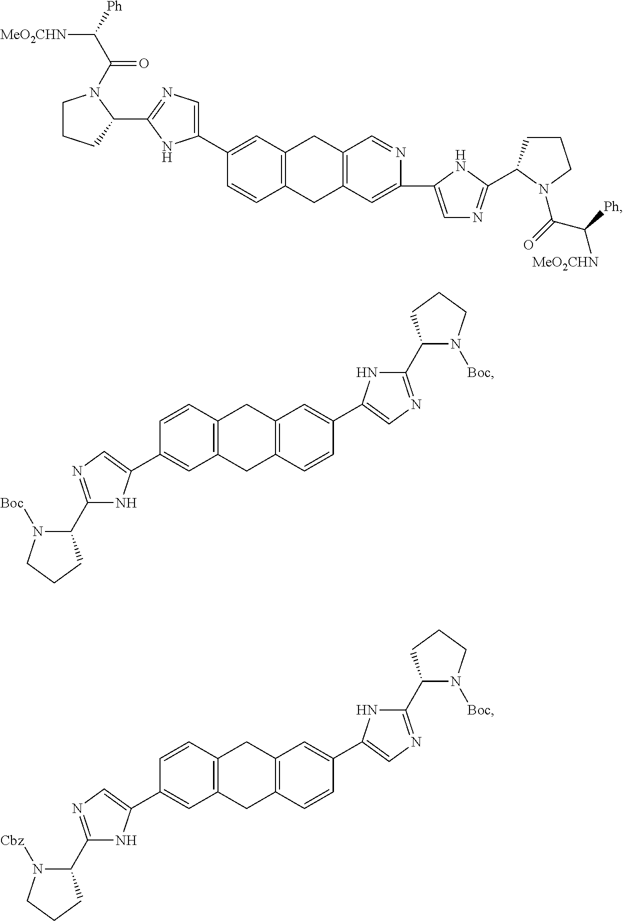

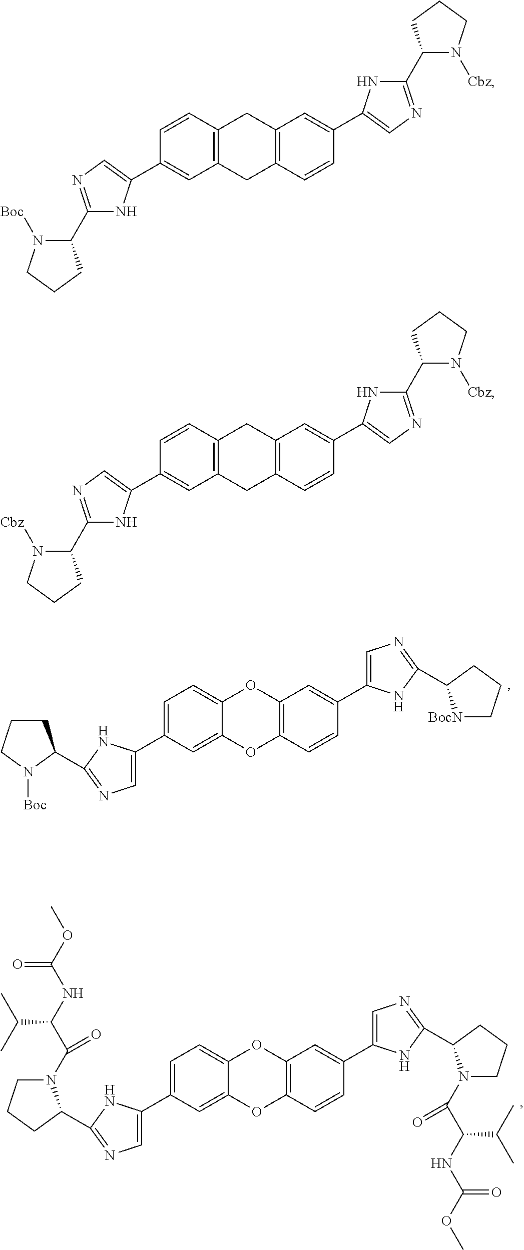

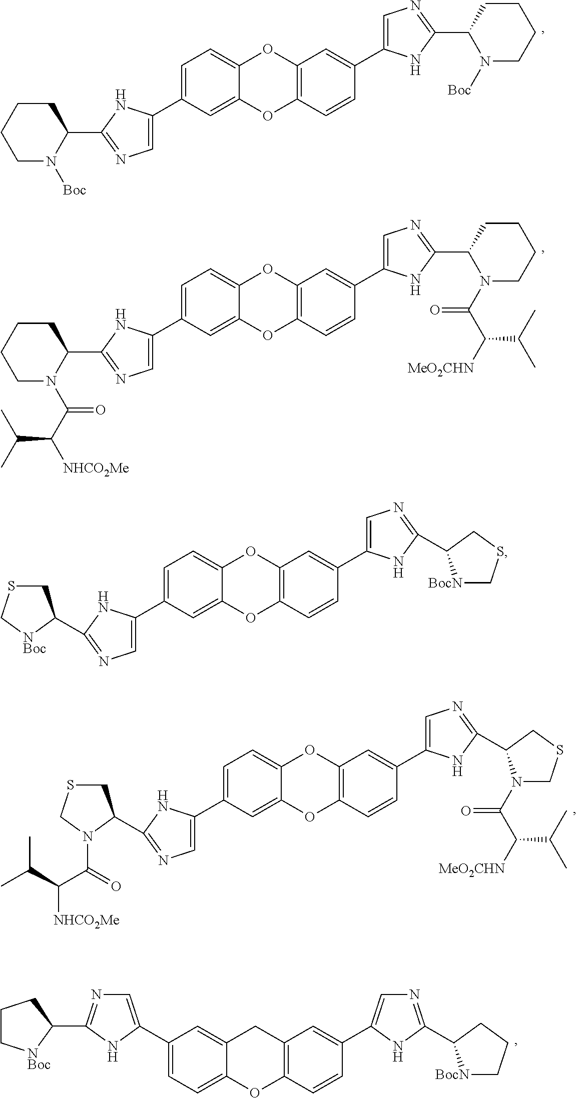

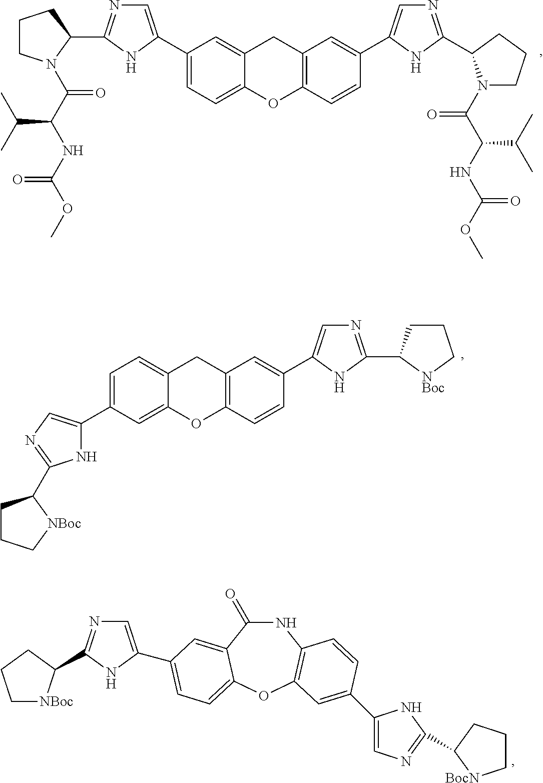

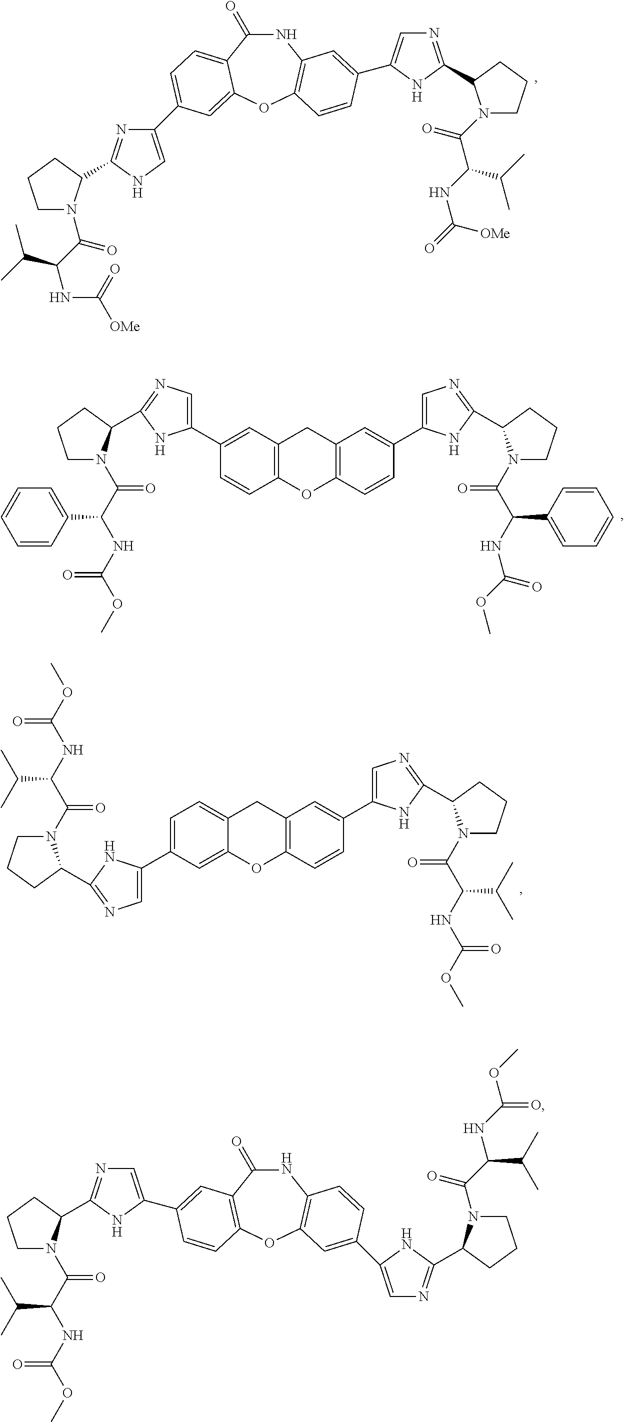

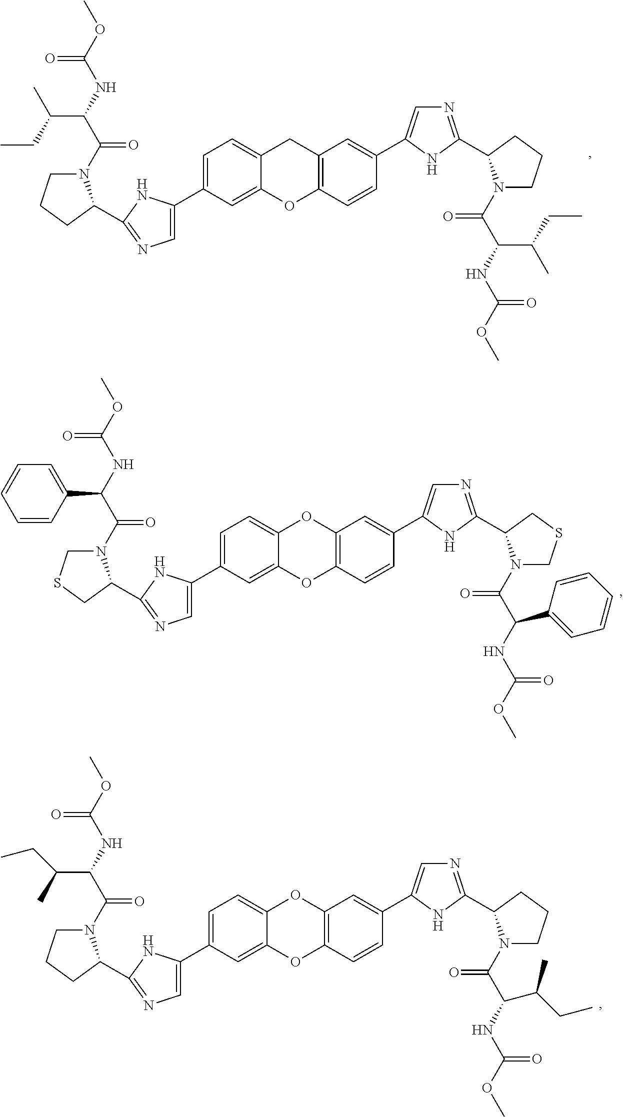

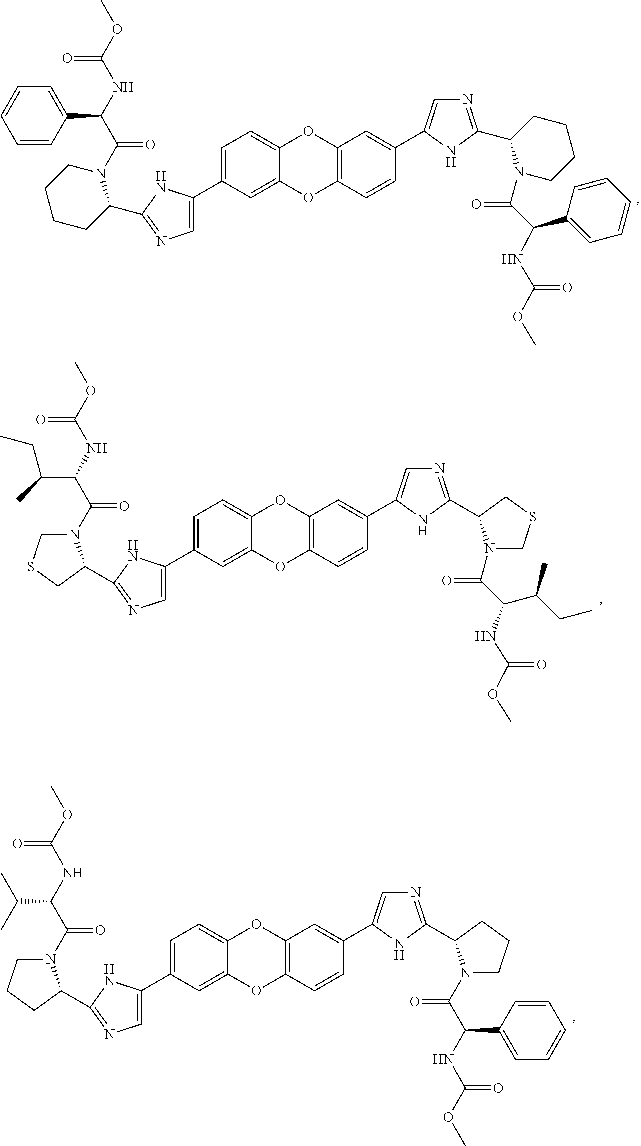

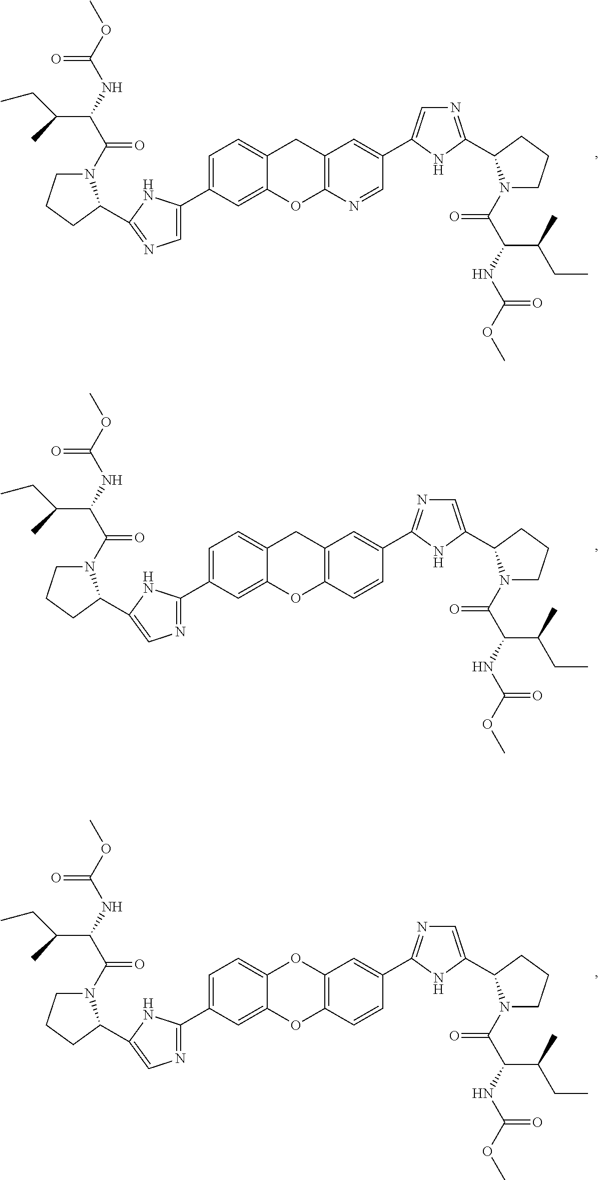

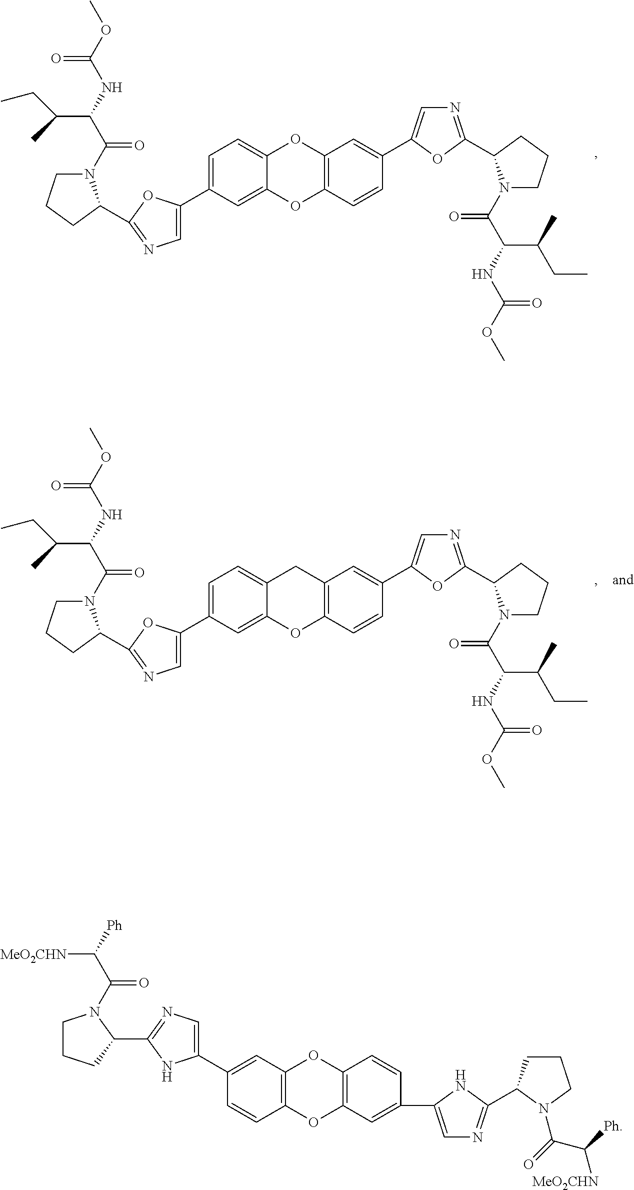

Still other embodiments include a compound having the structure of Formula V:

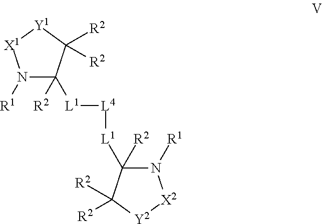

or a pharmaceutically acceptable salt thereof,

wherein:

each R¹ is separately selected from the group consisting of hydrogen and R^{1a}C(═O)— and R^{1a}C(═S)—;

each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;

each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;

each R^{e}R^{f}N is separately selected, wherein R^{e} and R^{f} are each separately selected from the group consisting of hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;

each R^{x}R^{y}N is separately selected, wherein R^{x} and R^{y} are each separately selected from the group consisting of hydrogen, C₁₋₆alkylOC(═O)—, C₁₋₆alkyl, C₁₋₆alkylC(═O)—, aryl, arylalkyl, cycloalkyl, and heterocyclyl;

each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is

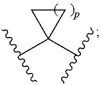

each R^{3a} is separately selected from the group consisting of hydrogen, and optionally substituted C₁₋₆alkyl;

each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;

each R^{4a}R^{4b}N is separately selected, wherein R^{4a} and R^{4b} are each separately selected from the group consisting of hydrogen, optionally substituted C₁₋₆alkyl, and aryl(CH₂)ₙ—;

each R^{5a} is separately selected from the group consisting of optionally substituted C₁₋₆alkyl, and aryl(CH₂)ₙ—;

each R^{6a} is separately selected from the group consisting of optionally substituted C₁₋₆alkyl, and aryl(CH₂)ₙ—;

X¹ is (C(R²)₂)_{q},

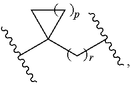

or X¹ is null;

Y¹ is selected from O (oxygen), S (sulfur), S(O), SO₂, NR², and C(R²)₂ with the proviso that when X¹ is null Y¹ is C(R²)₂;

X² is (C(R²)₂)_{q},

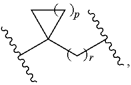

or X² is null;

Y² is selected from O (oxygen), S (sulfur), S(O), SO₂, NR², and C(R²)₂ with the proviso that when X² is null Y² is C(R²)₂;

each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups;

each R^{a}R^{b}N is separately selected, wherein R^{a} and R^{b} are each separately selected from the group consisting of hydrogen, C₂₋₆alkenyl, and C₁₋₆alkyl;

each A is separately selected from the group consisting of CR³ and N (nitrogen);

each L¹ is separately selected from the group consisting of

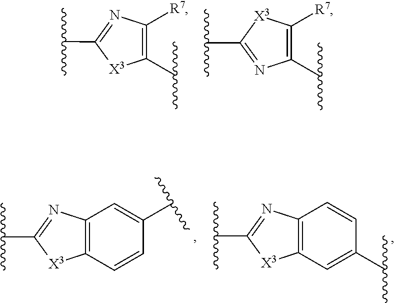

—C(═O)(CH₂)ₘOC(═O)—, —C(CF₃)₂NR^{2c}—, and

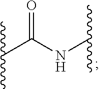

each X³ is separately selected from the group consisting of NH, NC₁₋₆alkyl, O (oxygen), and S (sulfur);

L⁴ is selected from the group consisting of

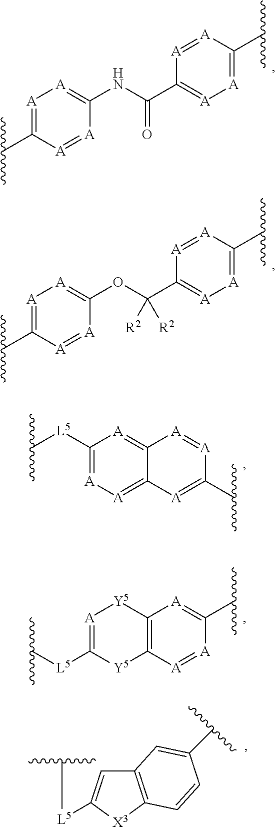

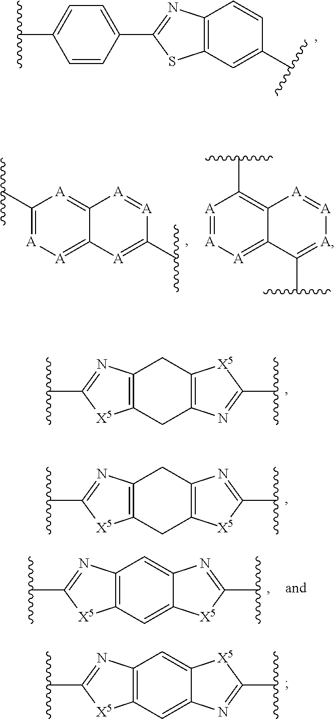

L⁵ is selected from the group consisting of

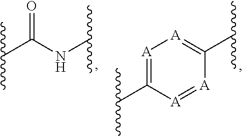

### and —(CH═CH)—;

each X⁵ is separately selected from the group consisting of —NH—, O (oxygen), S (sulfur), and —CH₂—,

each Y⁵ is separately selected from the group consisting of O (oxygen), S (sulfur), S(O), SO₂, NR², and C(R²)₂;

each m separately is 1 or 2;

each n separately is 0, 1 or 2;

each p separately is 1, 2, 3 or 4;

each q separately is 1, 2, 3, 4 or 5;

each r separately is 0, 1, 2, 3, or 4;

each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 9 halo and up to 5 hydroxy; and

each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo.

In some embodiments of Formula III, each R^{1a} is separately selected from the group consisting of —C(R^{2a})₂NR^{3a}R^{3b}, C₁₋₆alkylOC₁₋₆alkyl, C₁₋₆alkylOC(═O)—, C₁₋₆alkylOC(═O)C₁₋₆alkyl, C₁₋₆alkylC(═O)C₁₋₆alkyl, aryl, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, (R^{c}R^{d}N)(CH═CH)ₘ—, (R^{c}R^{d}N)alkyl, (R^{c}R^{d}N)C(═O)—, C₁₋₆alkoxy optionally substituted with up to 5 halo, and C₁₋₆alkyl optionally substituted with up to 5 halo;

each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 5 halo, and C₁₋₆alkyl optionally substituted with up to 5 halo;

each R^{2a} is separately selected from the group consisting of hydrogen, C₁₋₆alkyl, aryl(CH₂)ₙ—, and heteroaryl(CH₂)ₙ—;

each R^{3a} is separately selected from the group consisting of hydrogen, and C₁₋₆alkyl;

each R^{3b} is separately selected from the group consisting of C₁₋₆alkyl, —(CH₂)ₙC(═O)NR^{4a}R^{4b}, —(CH₂)ₙC(═O)OR^{5a}, and —(CH₂)ₙC(═O)R^{6a};

each R^{4a}R^{4b}N is separately selected, wherein R^{4a} and R^{4b} are each separately selected from the group consisting of hydrogen, C₁₋₆alkyl, and aryl(CH₂)ₙ—;

each R^{5a} is separately selected from the group consisting of C₁₋₆alkyl, and aryl(CH₂)ₙ—;

each R^{6a} is separately selected from the group consisting of C₁₋₆alkyl, and aryl(CH₂)ₙ;

X¹ is C(R²)₂, or X¹ is null;

Y¹ is selected from O (oxygen), S (sulfur), S(O), SO₂, and C(R²)₂ with the proviso that when X¹ is null Y¹ is C(R²)₂;

X² is C(R²)₂, or X² is null;

Y² is selected from O (oxygen), S (sulfur), S(O), SO₂, and C(R²)₂ with the proviso that when X² is null Y² is C(R²)₂;

each X³ is separately selected from the group consisting of NH, O (oxygen), and S (sulfur);

each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 5 halo, or optionally two vicinal R² and the carbons to which they are attached are together a fused three- to six-membered carbocyclic ring optionally substituted with up to two C₁₋₆alkyl groups;

each L¹ is separately selected from the group consisting of

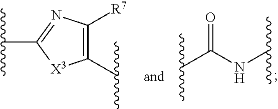

L₄ is selected from the group consisting of

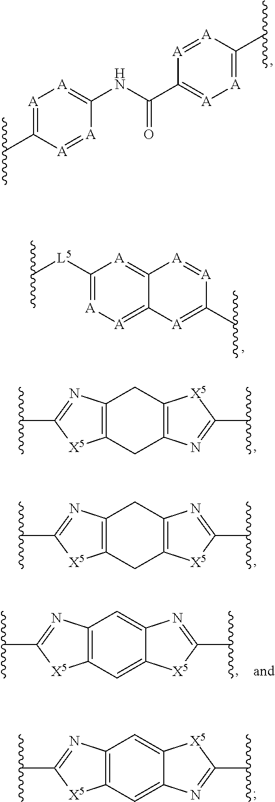

each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 5 halo and up to 5 hydroxy; and

each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 5 halo.

In some embodiments of Formula V, each L₁ is

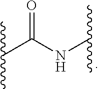

In some embodiments of Formula V, L⁴ is

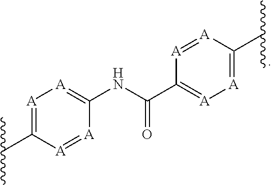

In some embodiments of Formula V, L⁴ is

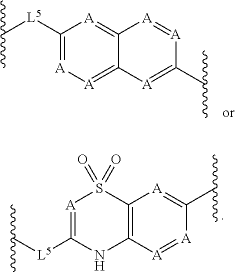

In some embodiments of Formula V, L⁴ is

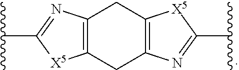

In some embodiments of Formula V, L⁴ is

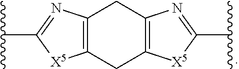

In some embodiments of Formula V, L⁴ is

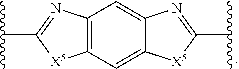

In some embodiments of Formula V, L⁴ is

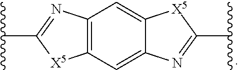

In some embodiments of Formula V, L⁴ is

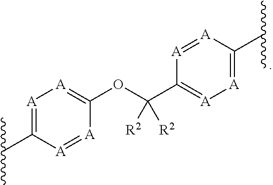

In some embodiments of Formula V, L⁵ is

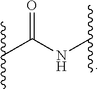

In some embodiments of Formula V, L⁵ is

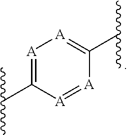

In some embodiments of Formula V, L⁵ is —(CH═CH)—.

In some embodiments, the compound of Formula V has the structure of one of the following formulas:

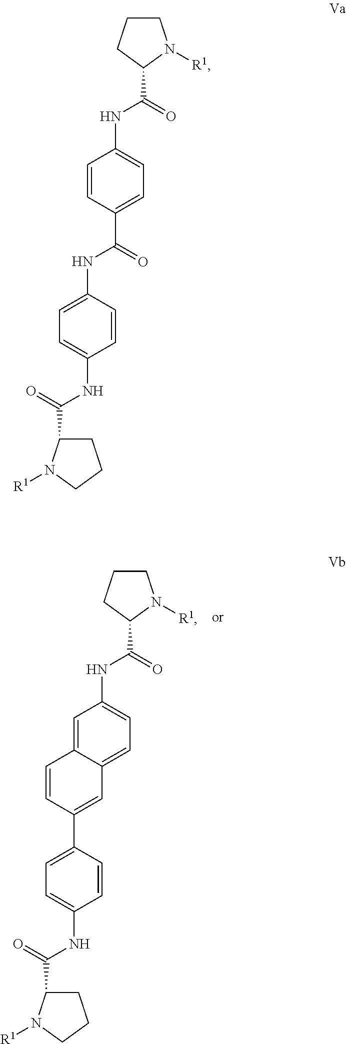

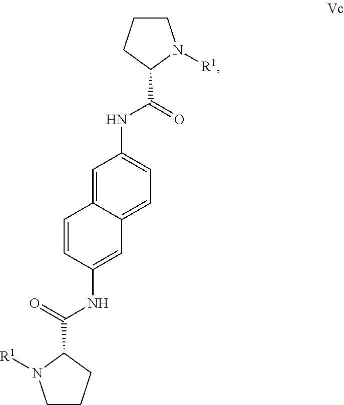

or pharmaceutically acceptable salts thereof.

In some embodiments, the compound of Formula V has the structure of Formula Vd:

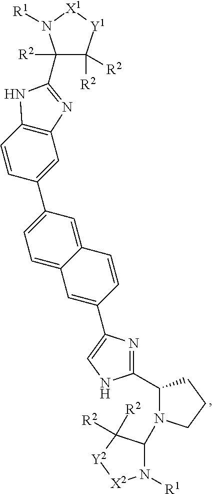

or pharmaceutically acceptable salts thereof.

In some embodiments, the compound of Formula V has the structure of Formula Vf:

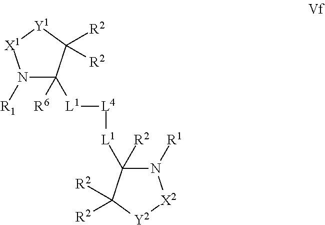

or a pharmaceutically acceptable salt thereof, wherein:

R⁶ is C₁₋₆alkyl optionally substituted with up to 9 halo.

In some embodiments of Formula V, Formula Va, Formula Vb, Formula Vc, Formula Vd, or Formula Vf, each R¹ is R^{1a}C(═O)—.

In some embodiments of Formula V, Formula Va, Formula Vb, Formula Vc, Formula Vd, or Formula Vf, each R^{1a} is —CHR^{2a}NHR^{3b}.

In some embodiments of Formula V, Formula Va, Formula Vb, Formula Vc, Formula Vd, or Formula Vf, each R^{2a} is C₁₋₆alkyl; each R^{3b} is —C(═O)OR⁵; and each R⁵ is C₁₋₆alkyl.

In some embodiments of Formula Vd, L⁴ is

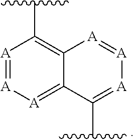

In some embodiments of Formula Vd, each L¹ is

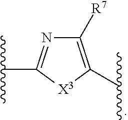

In some embodiments of Formula Vd, each L¹ is

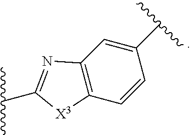

In some embodiments of Formula Vd, one L¹ is

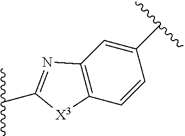

and the other L¹ is

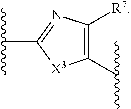

In some embodiments of Formula Vd, L⁵ is

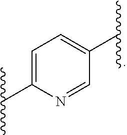

In some embodiments, the compound of Formula V has the structure

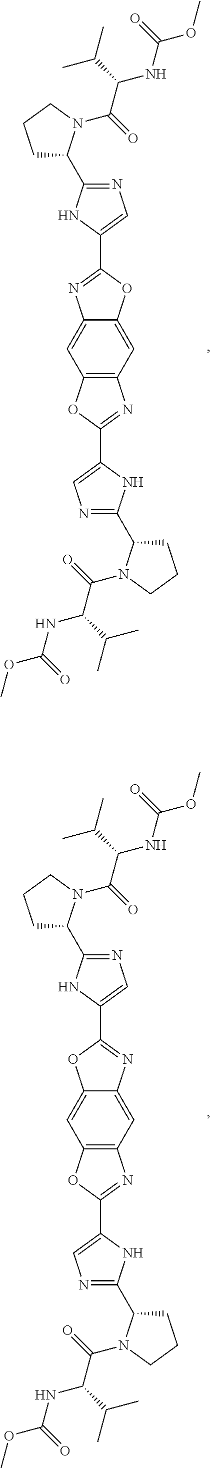

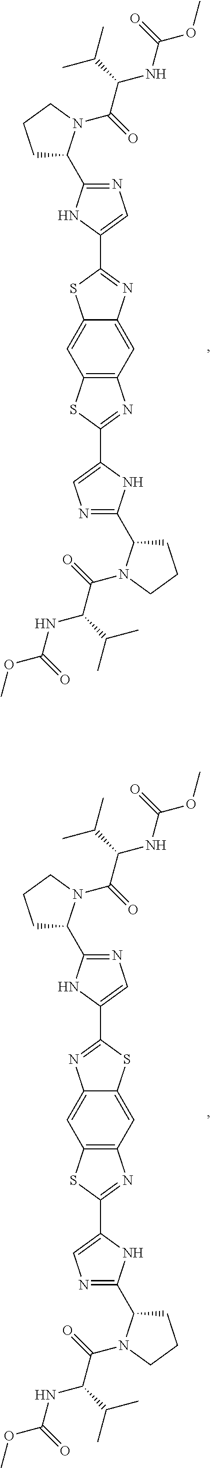

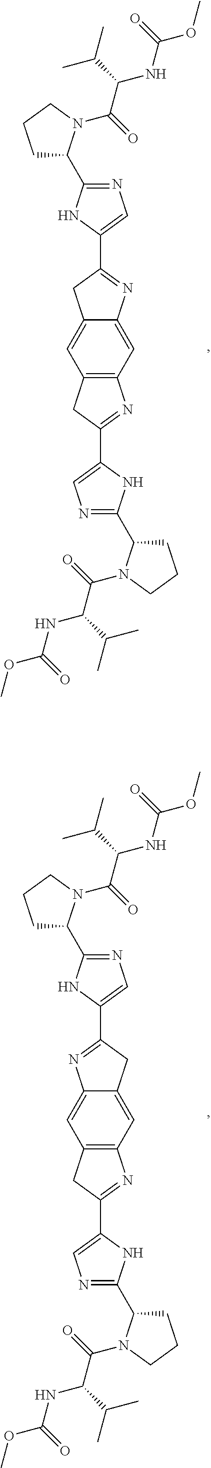

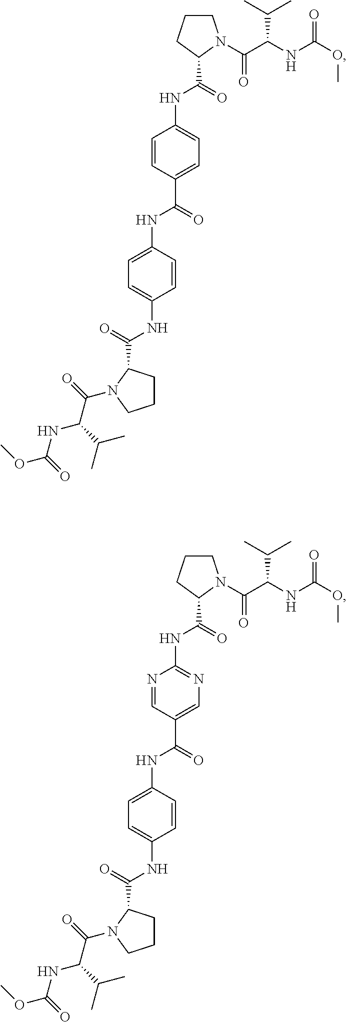

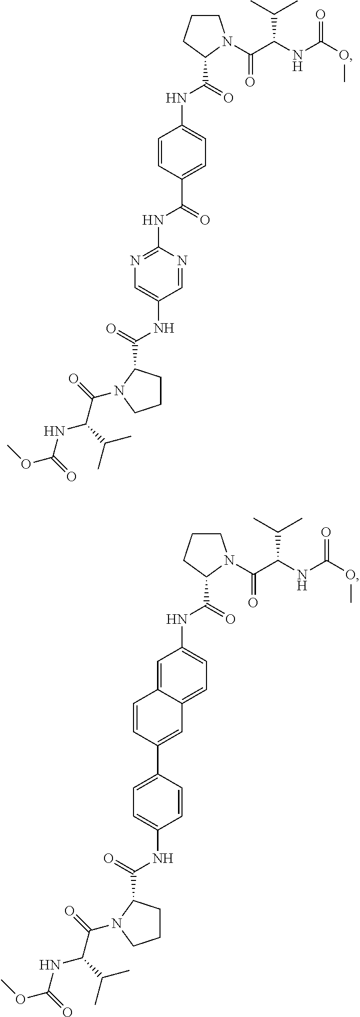

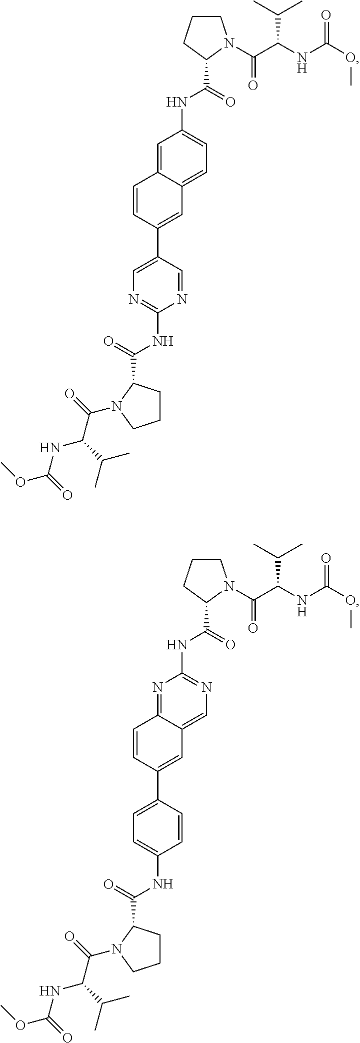

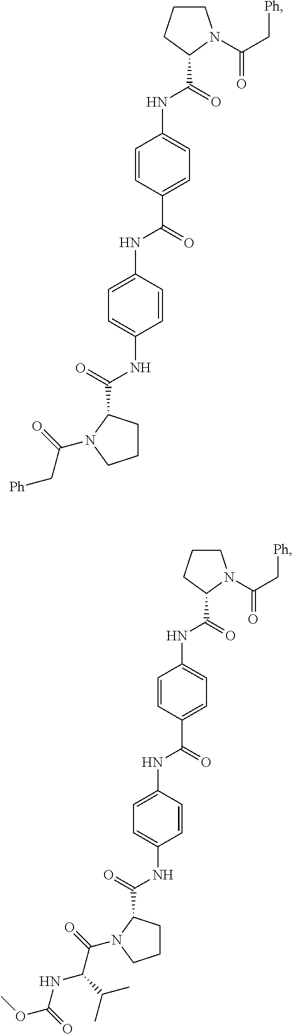

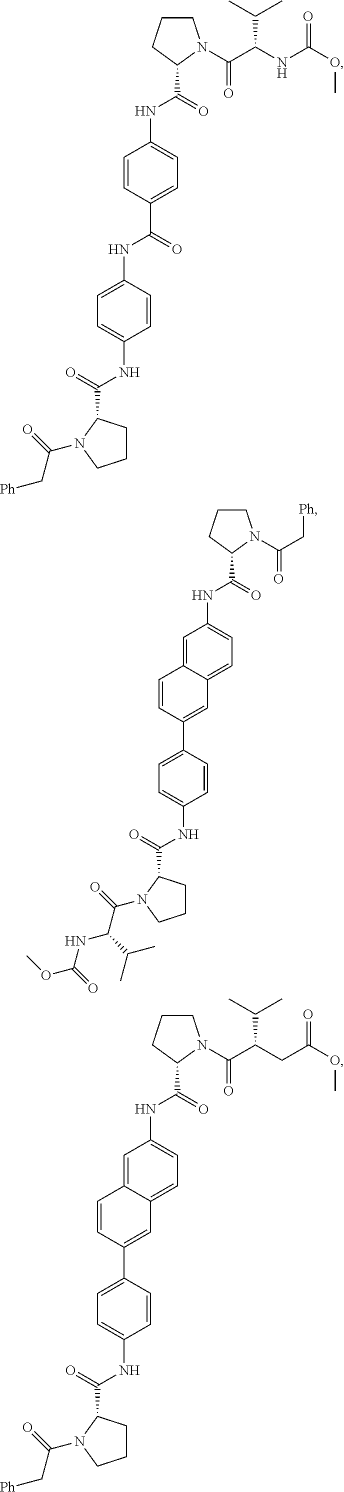

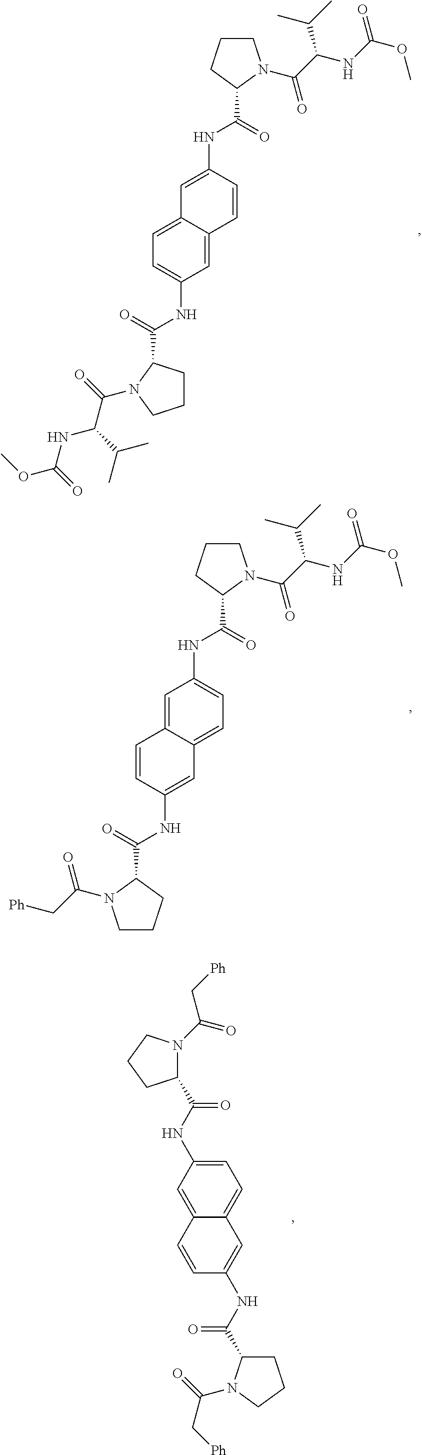

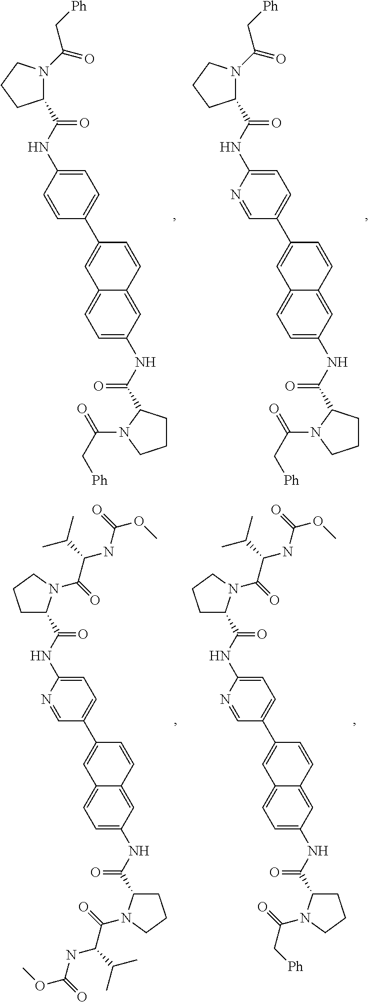

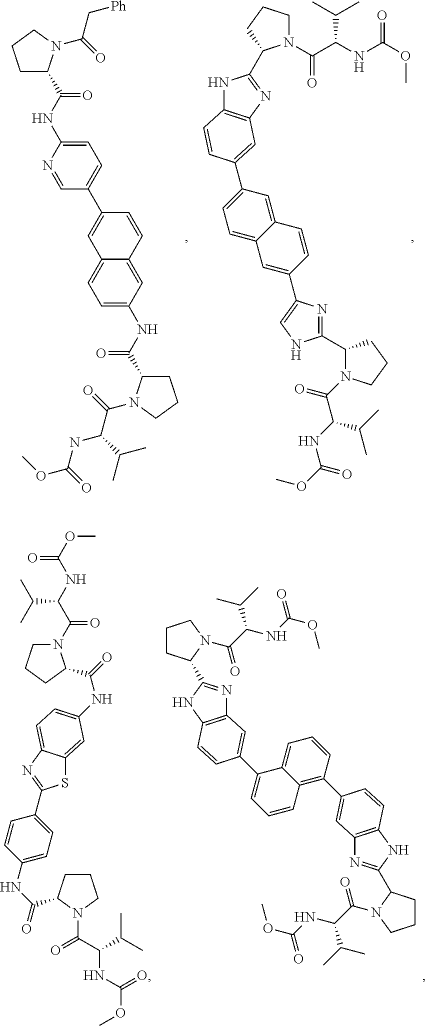

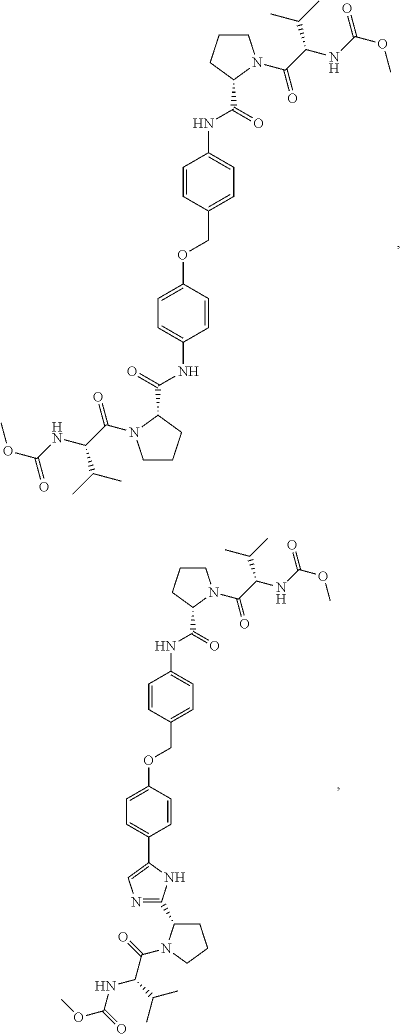

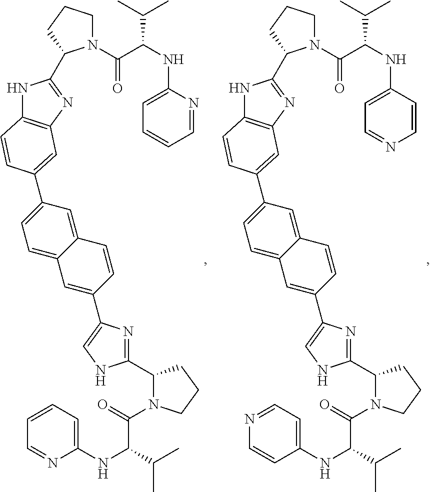

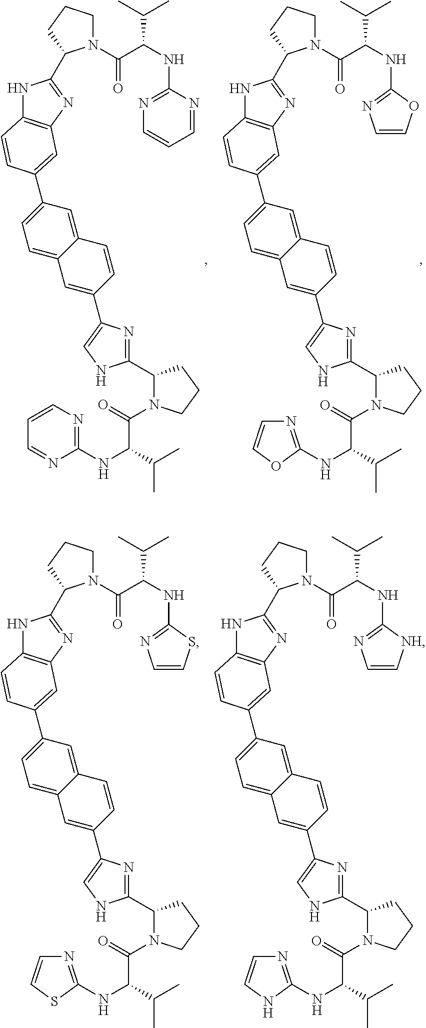

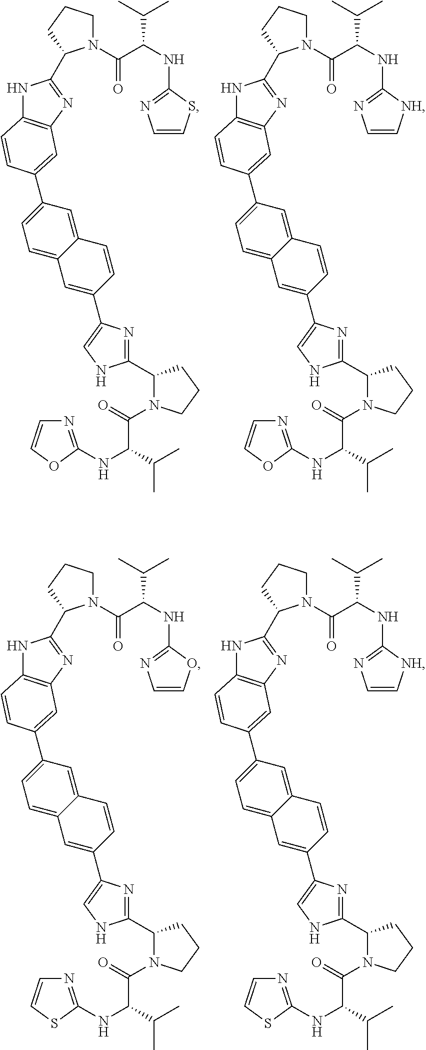

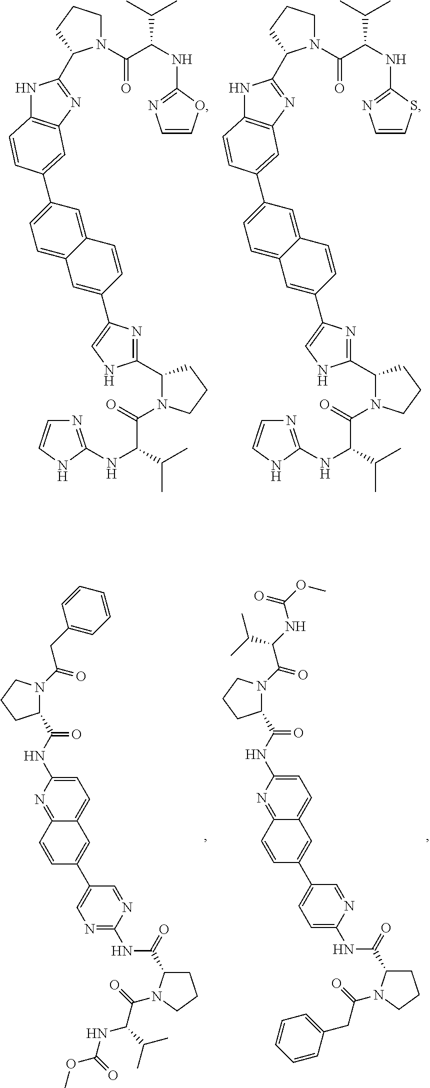

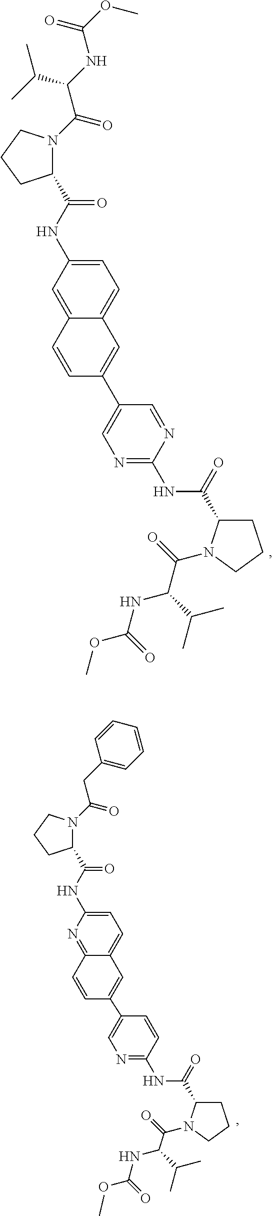

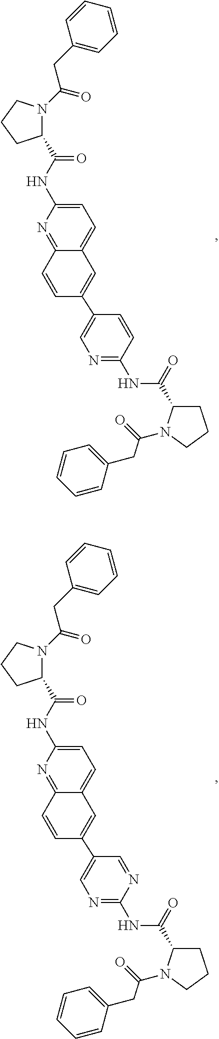

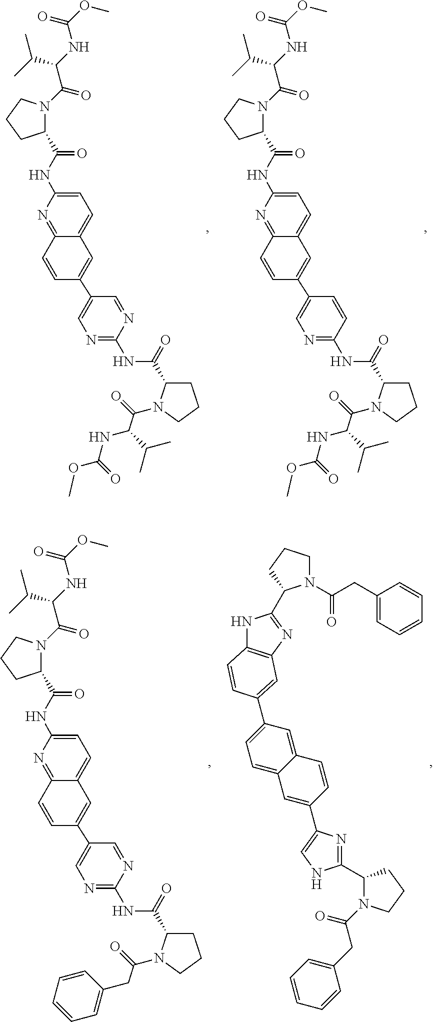

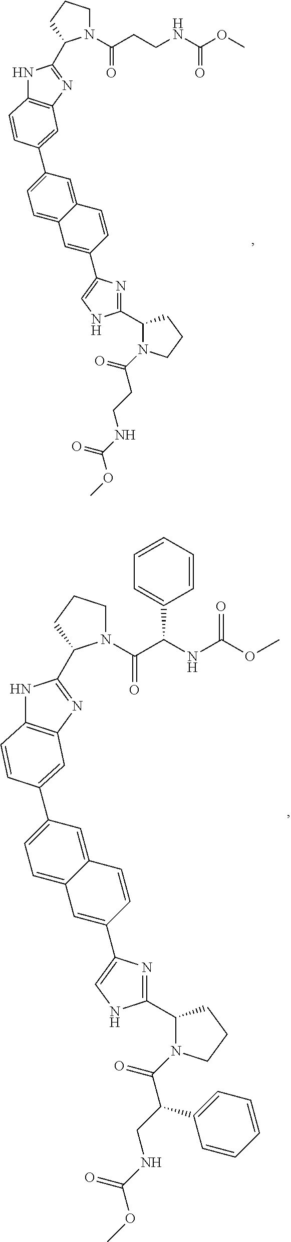

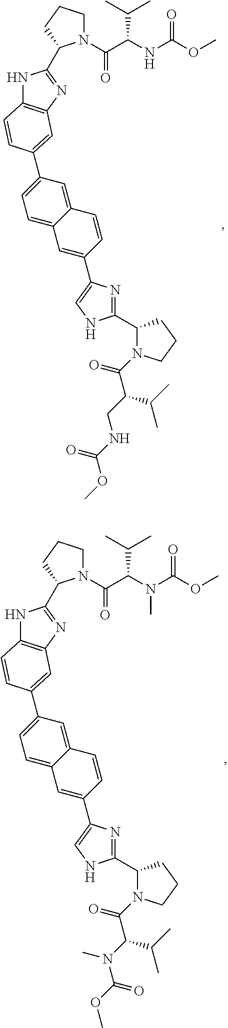

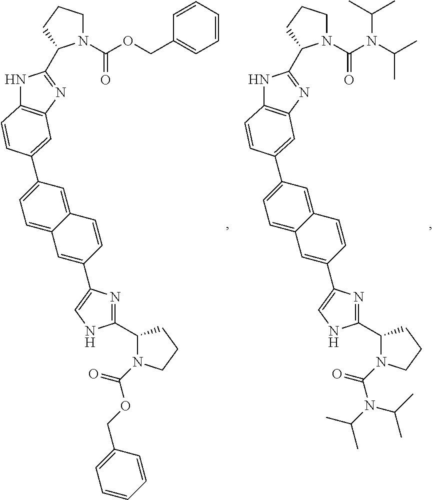

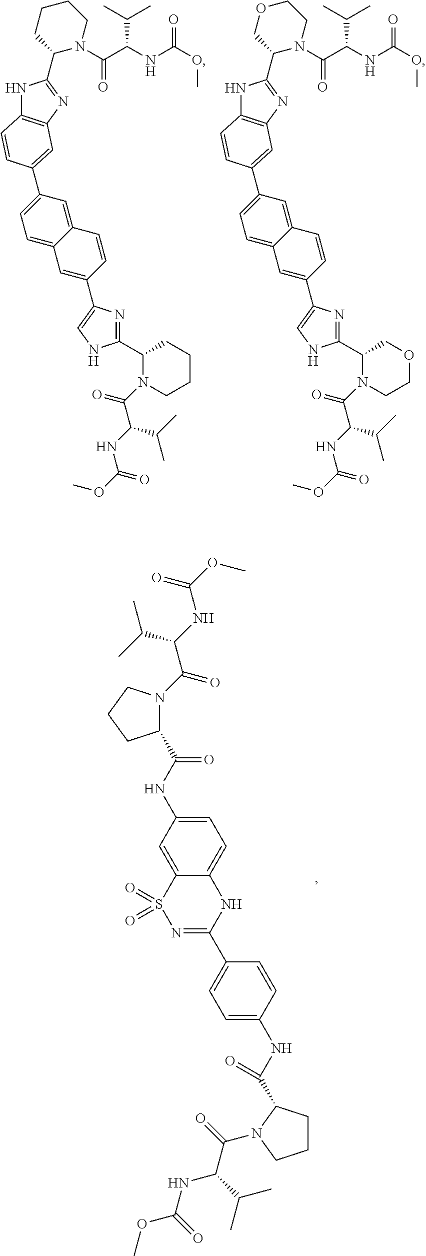

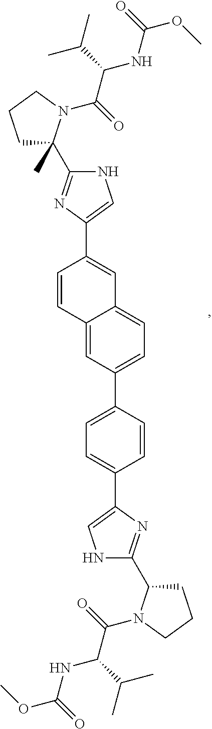

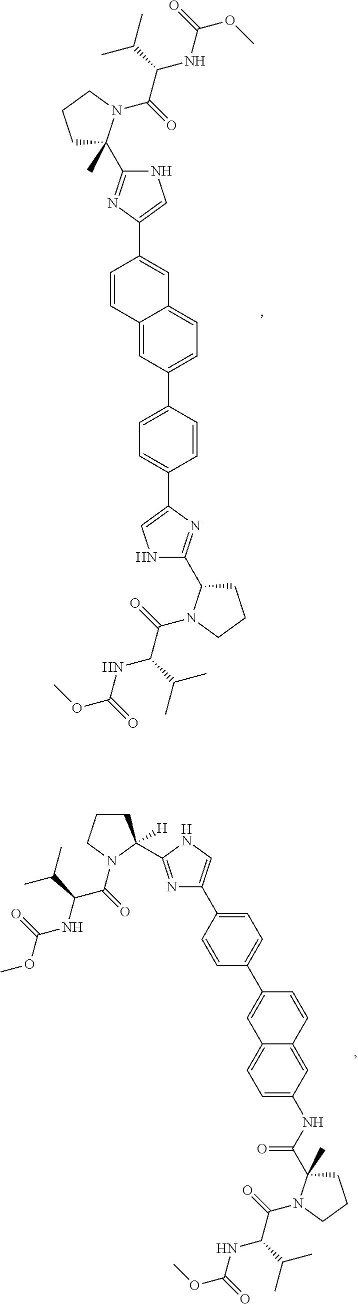

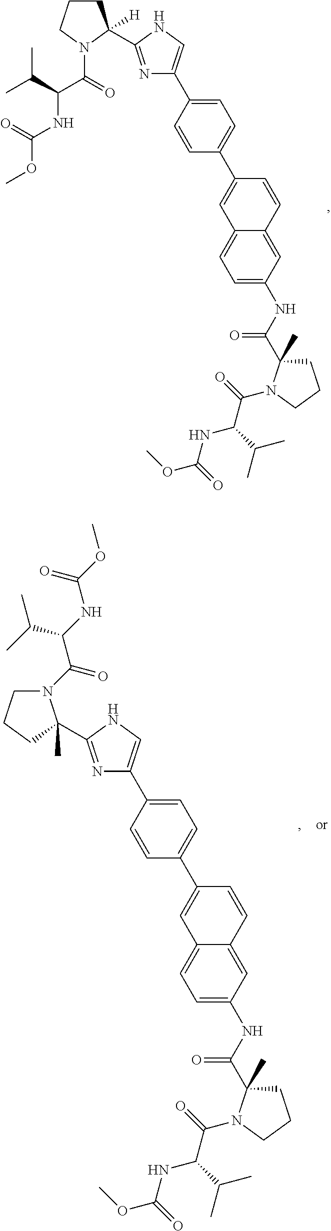

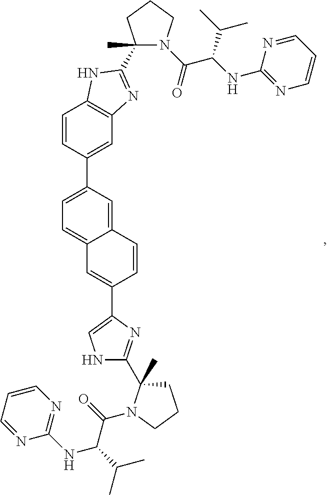

or a pharmaceutically acceptable salt thereof.

In some embodiments of Formula V, L⁴ is no

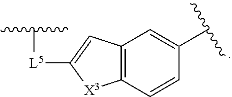

In some embodiments of Formula V, the compound is not selected from the group consisting of:

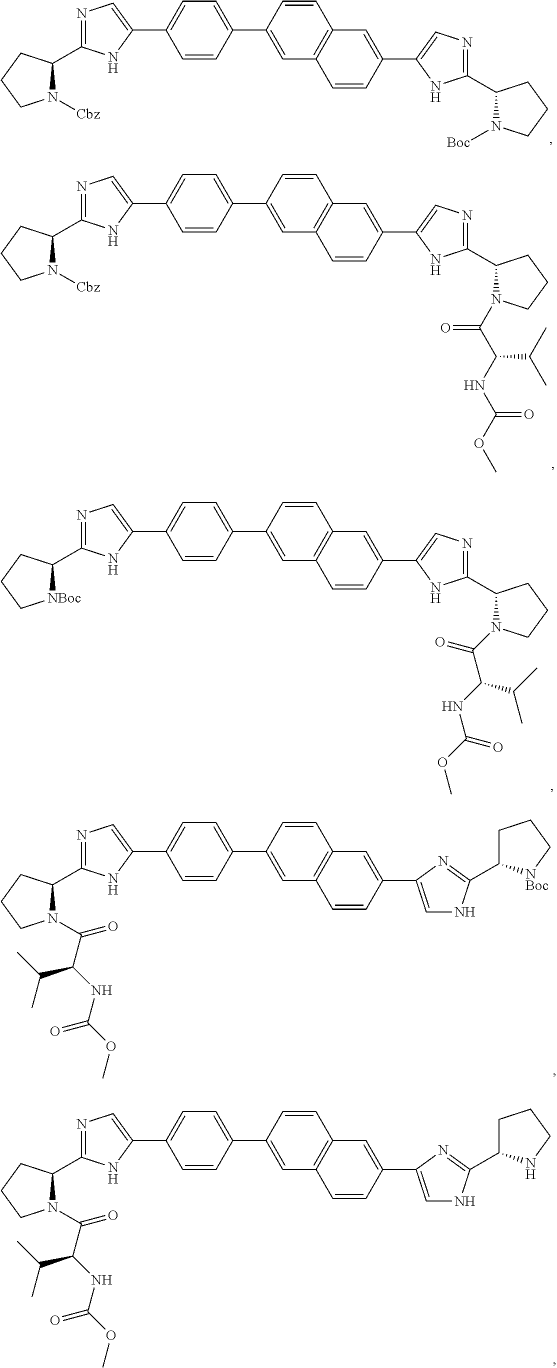

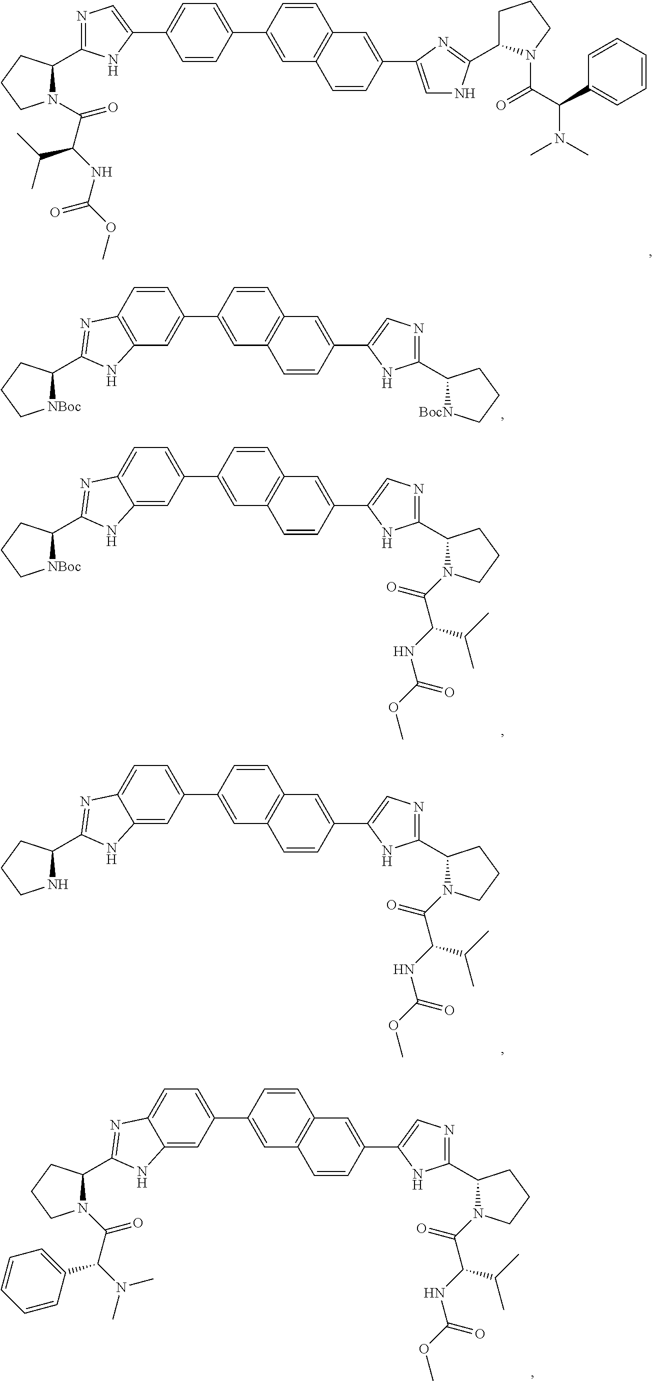

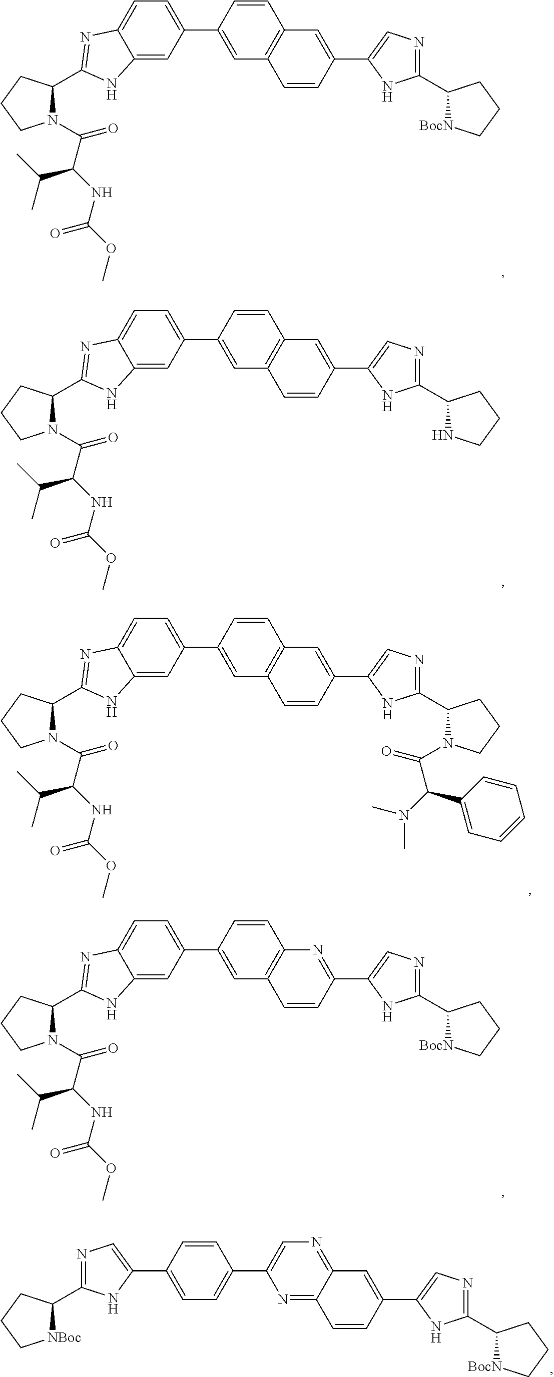

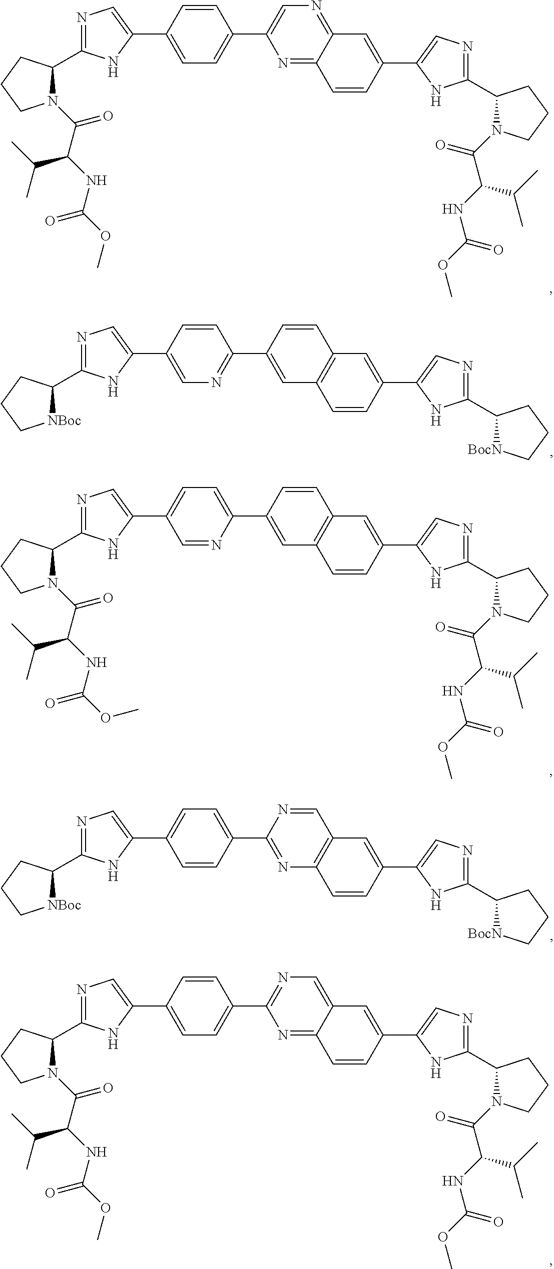

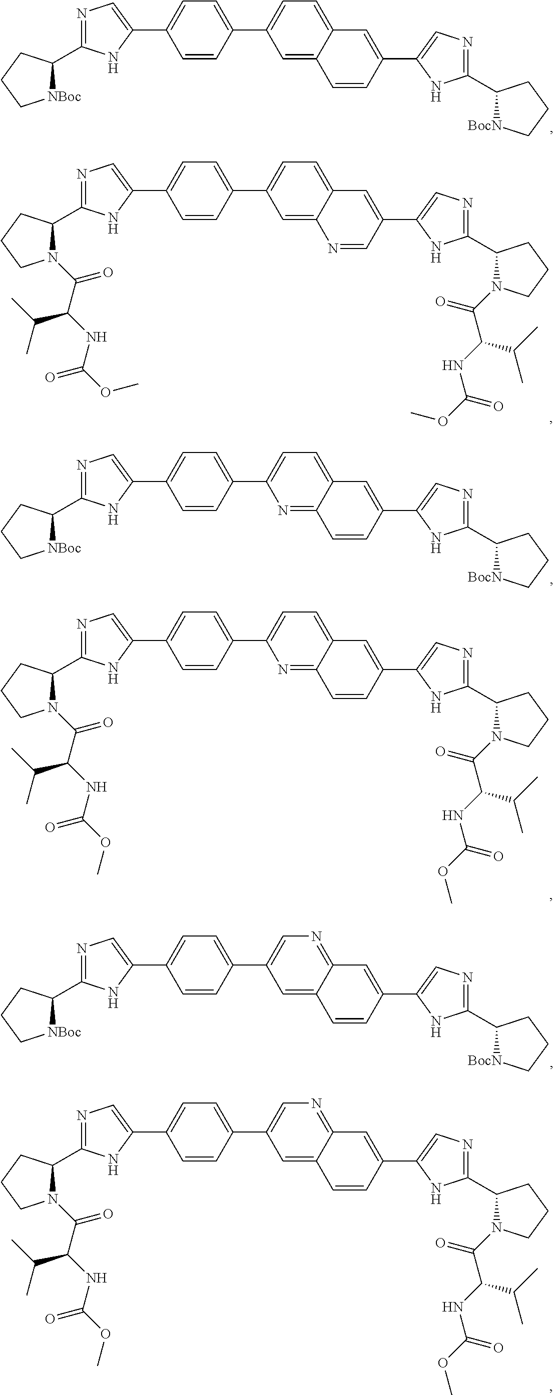

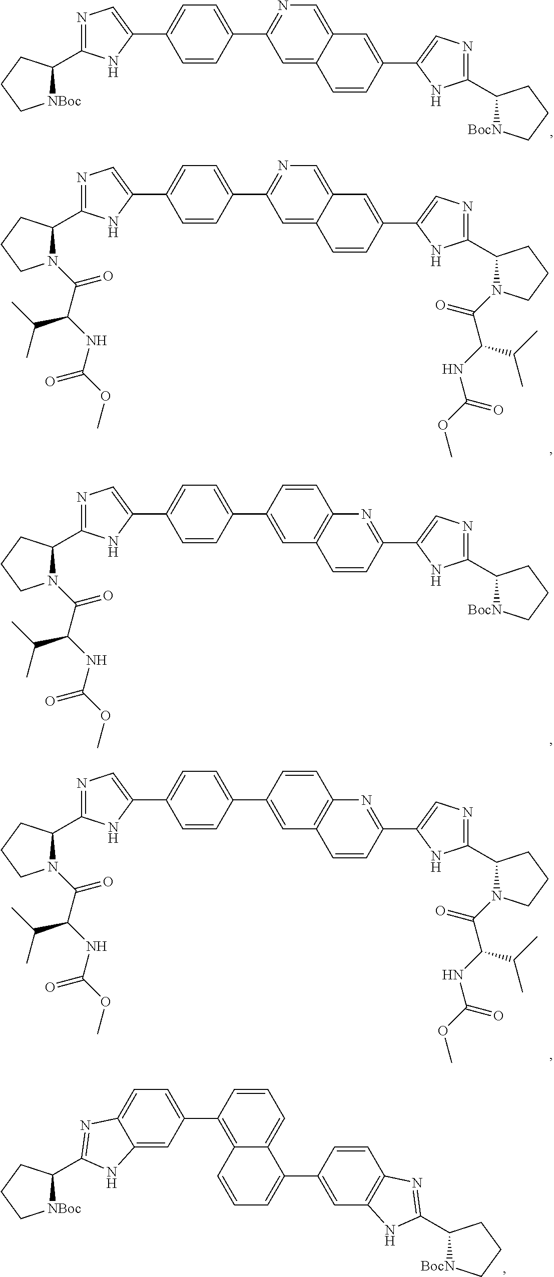

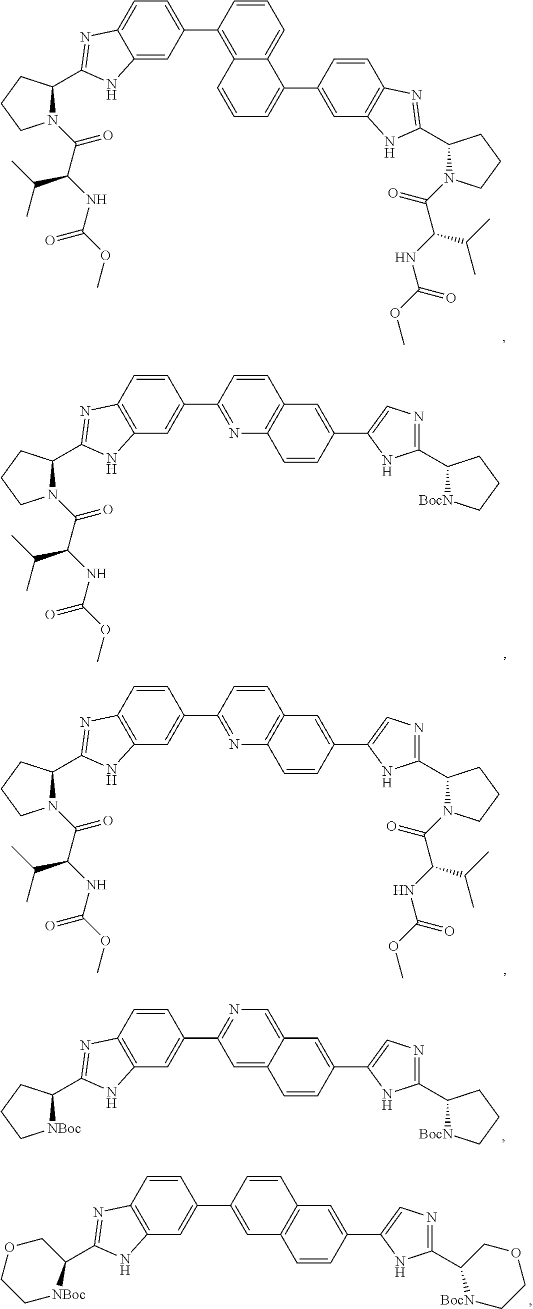

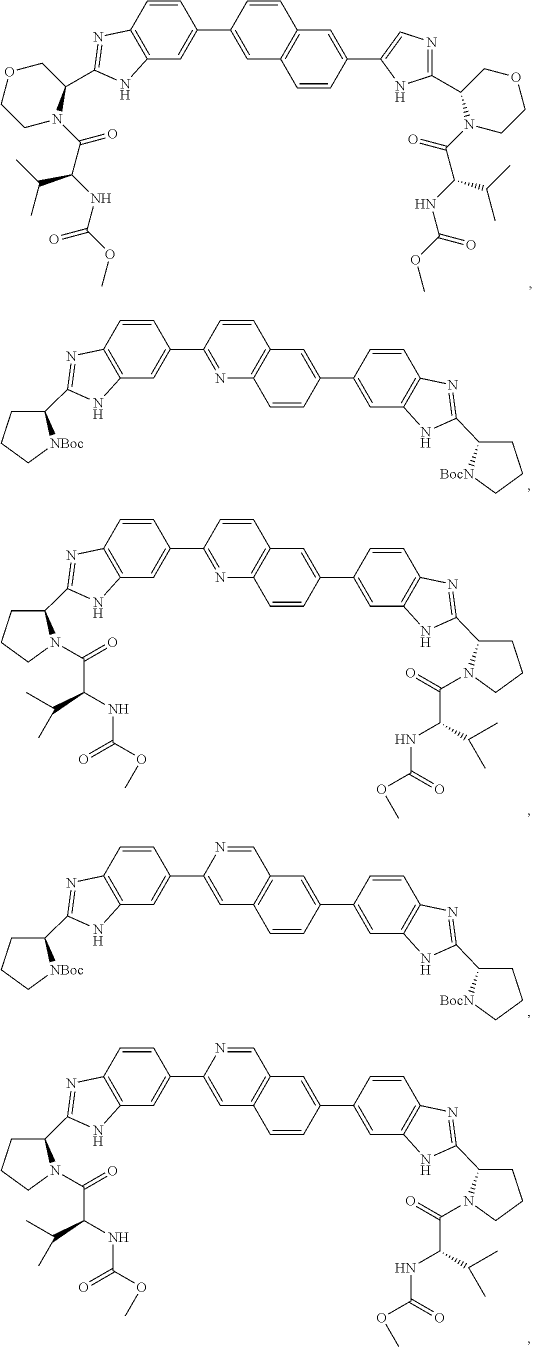

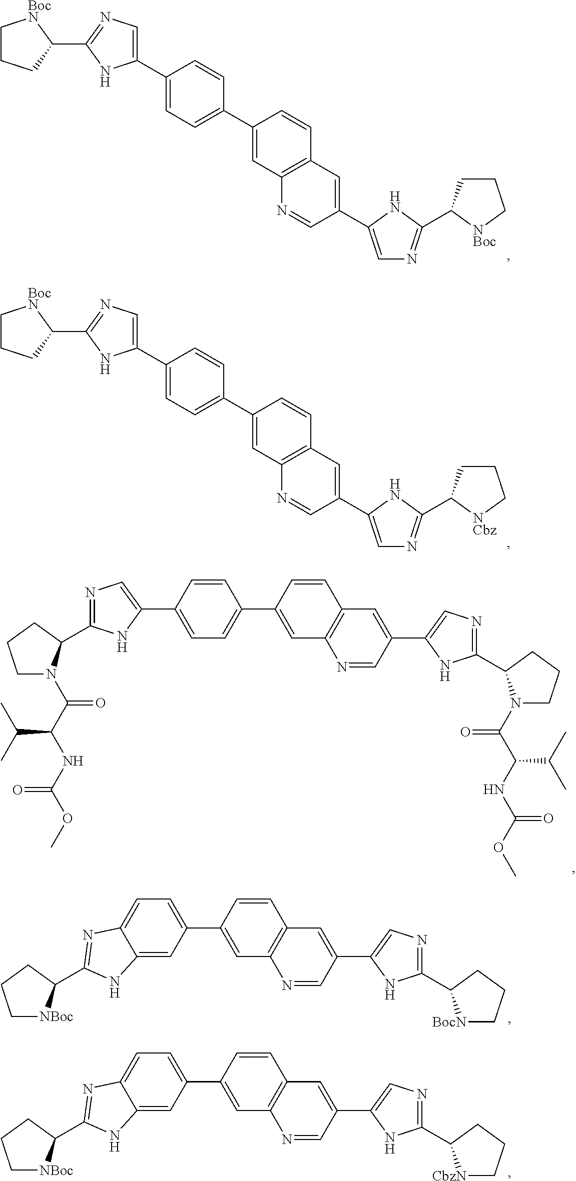

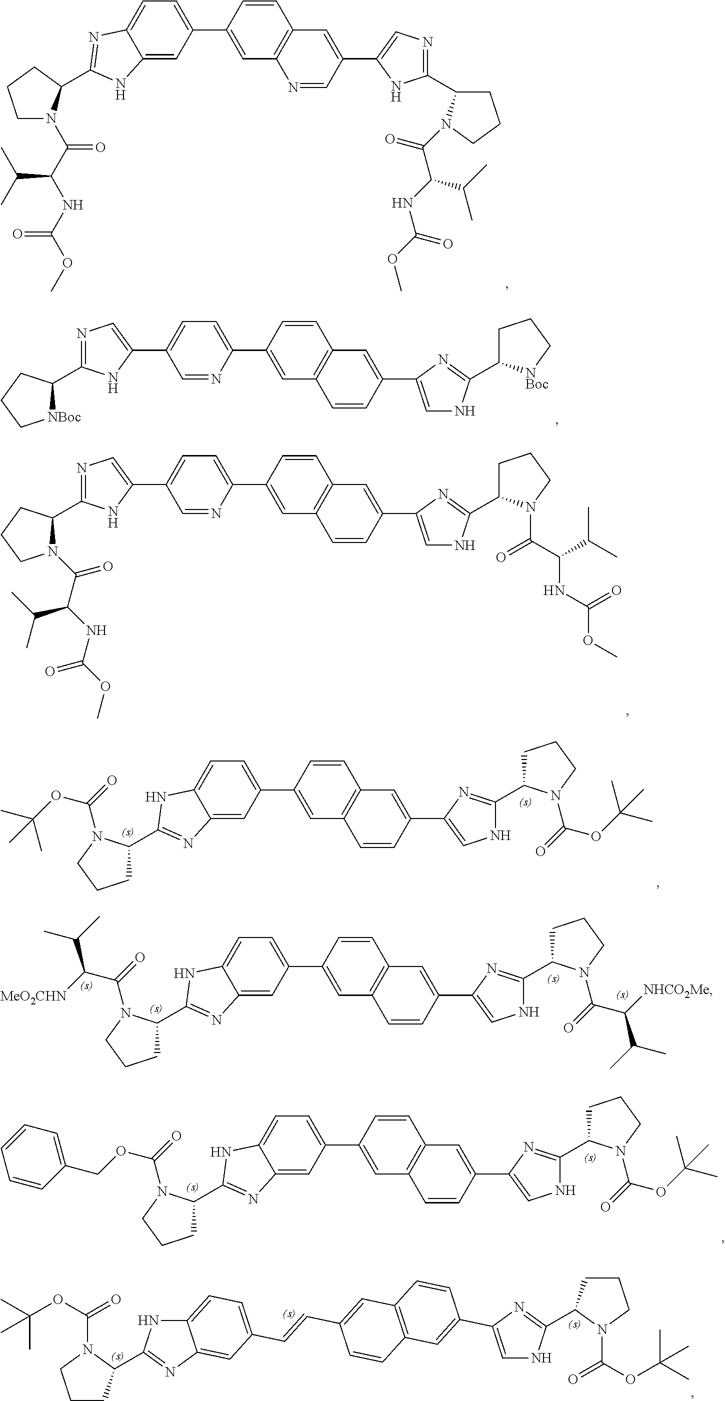

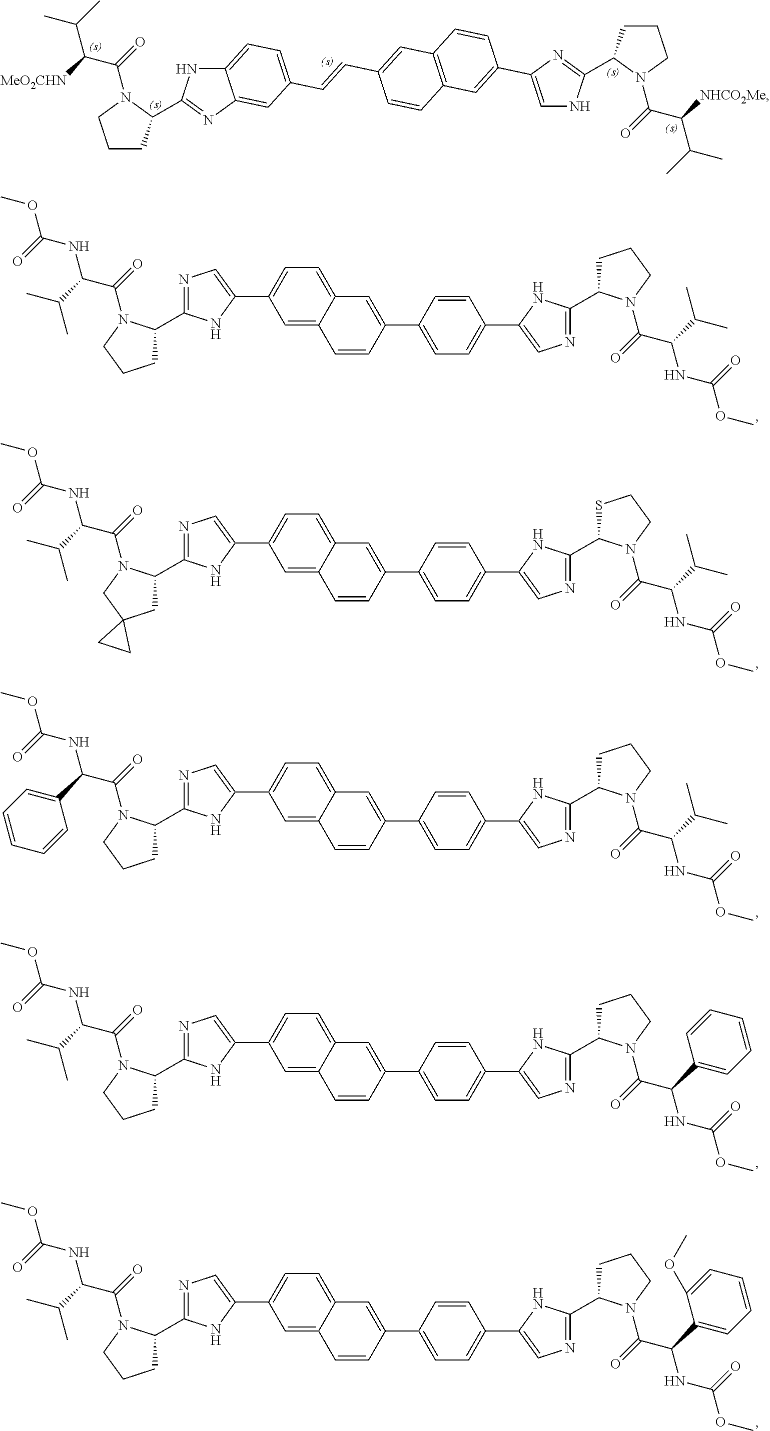

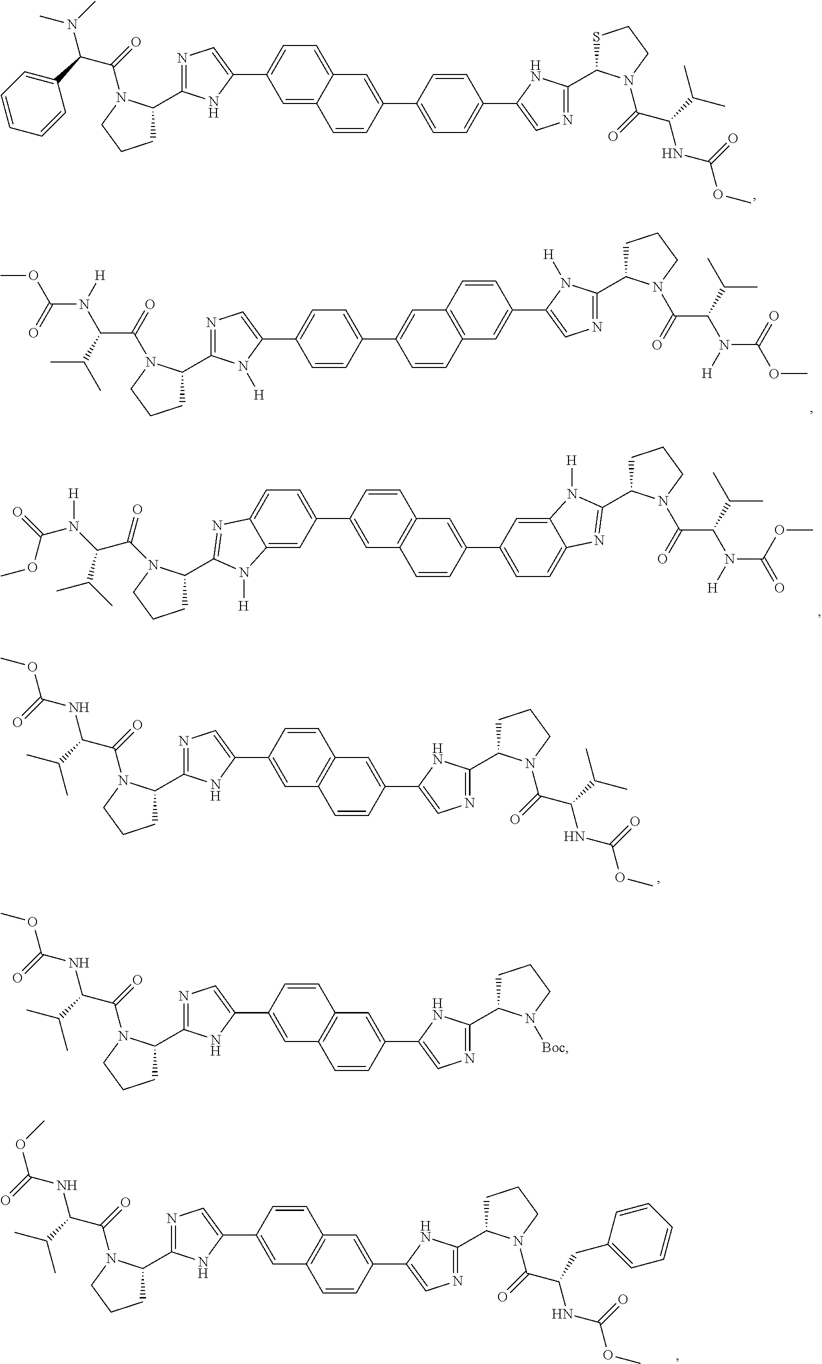

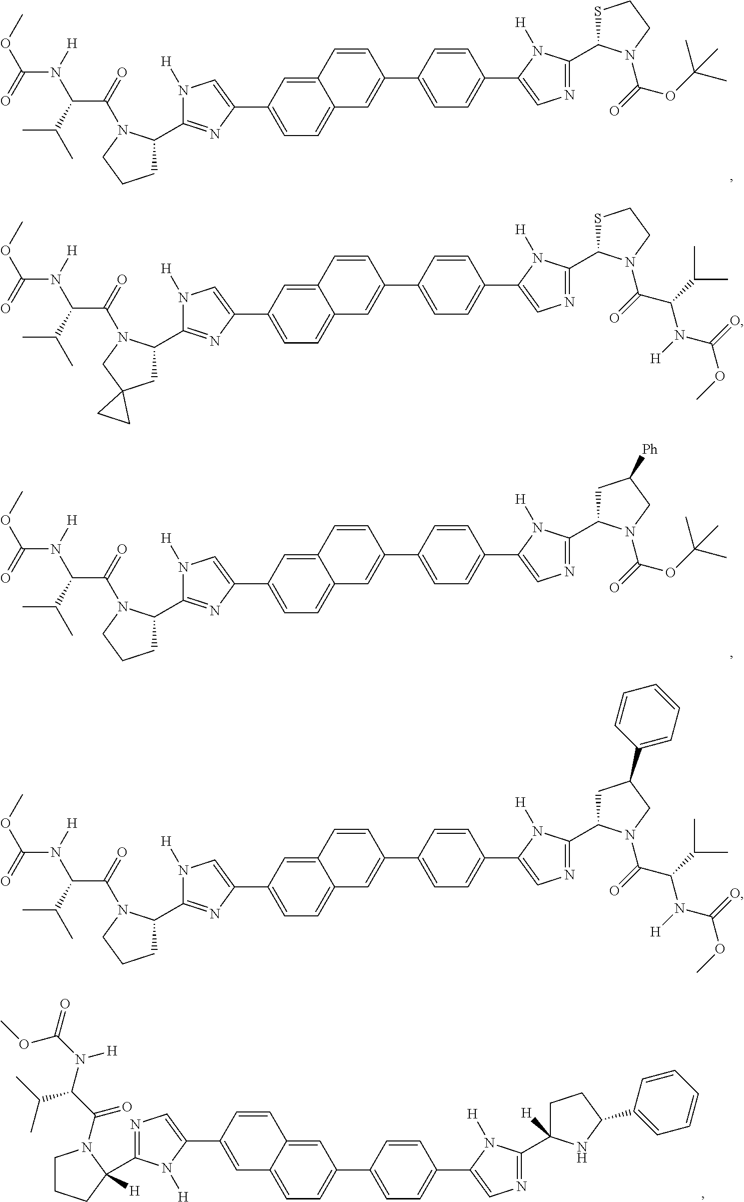

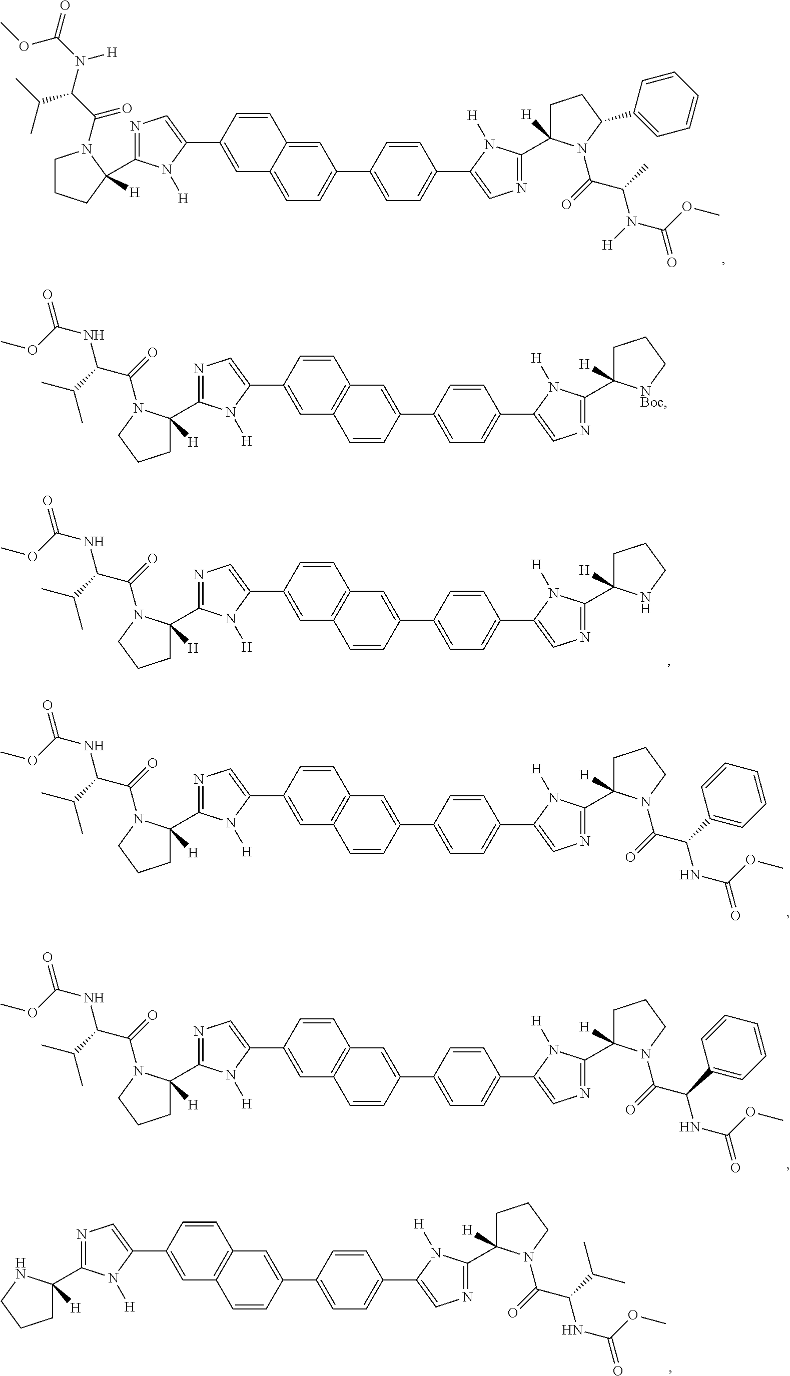

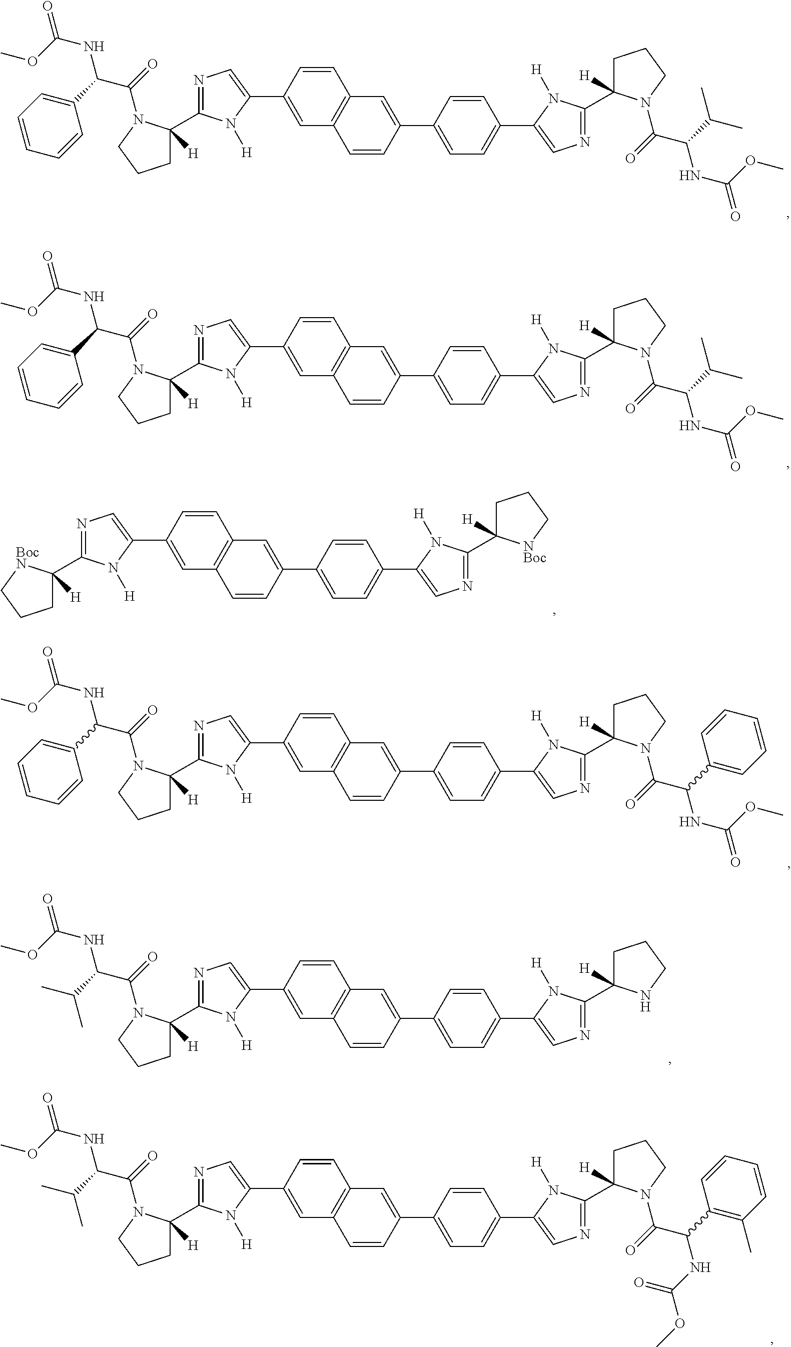

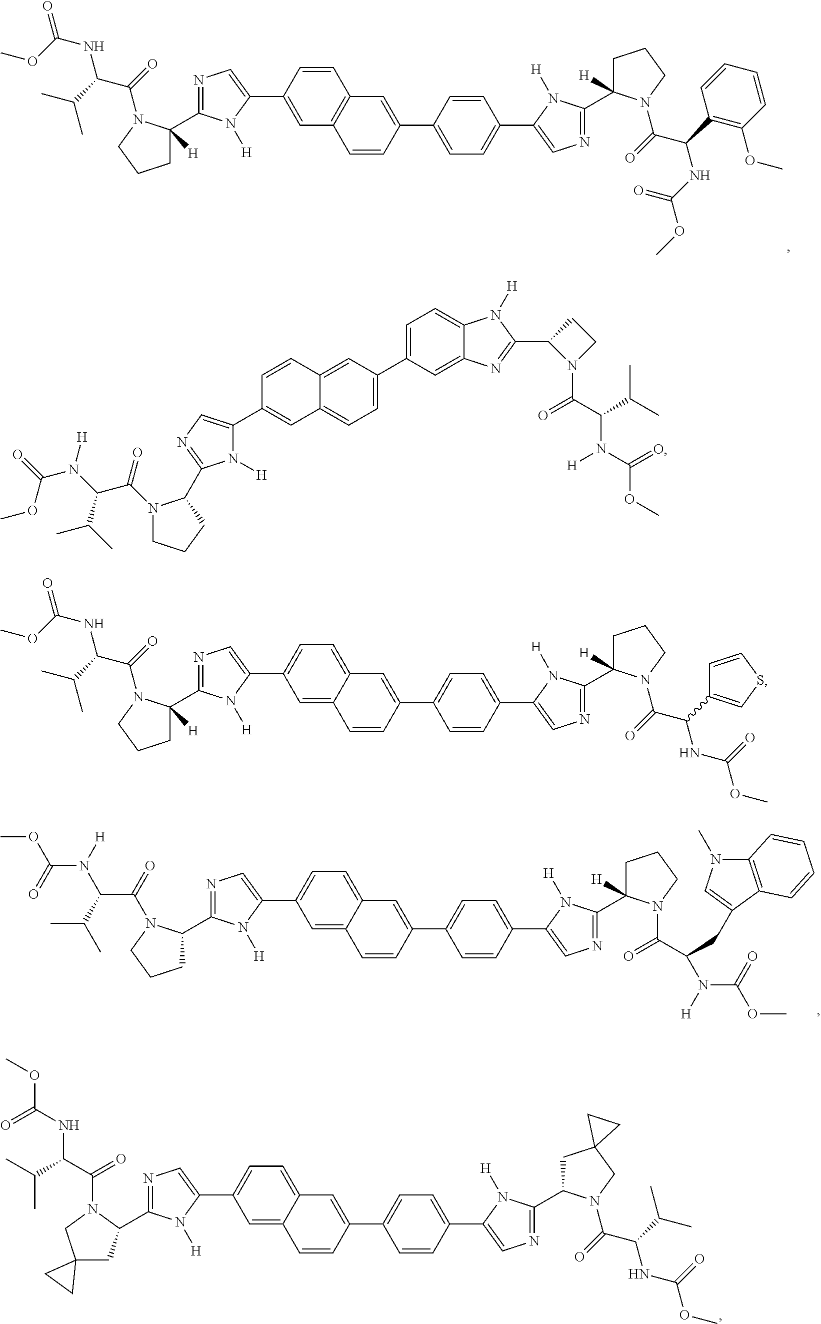

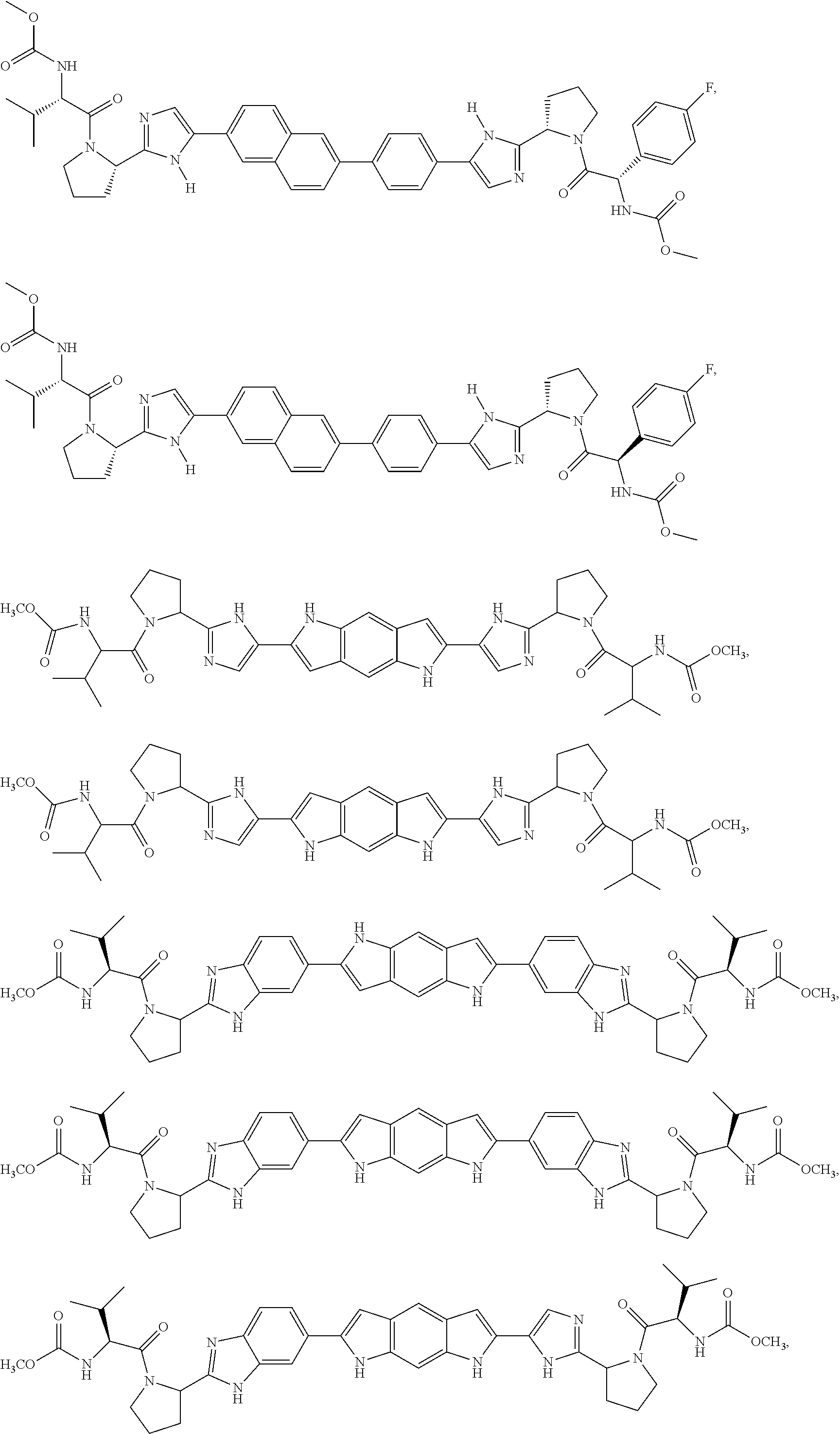

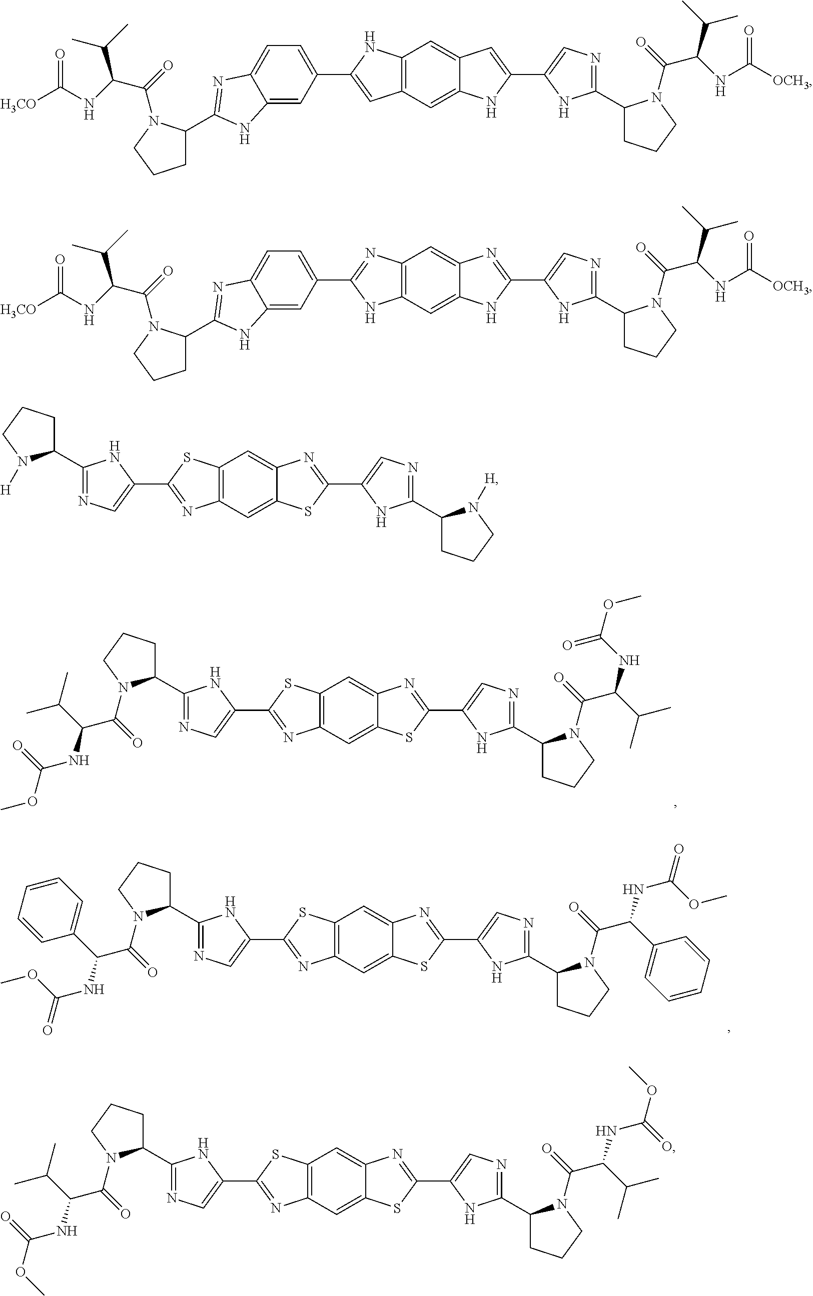

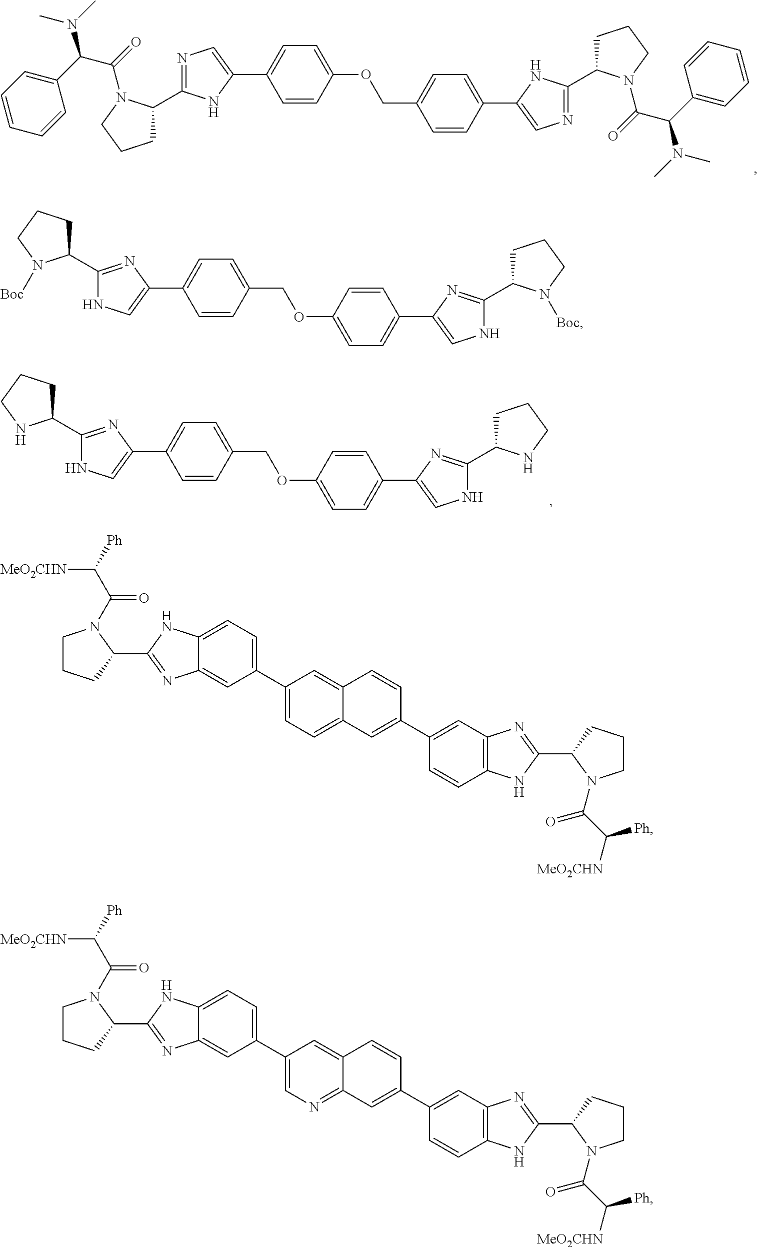

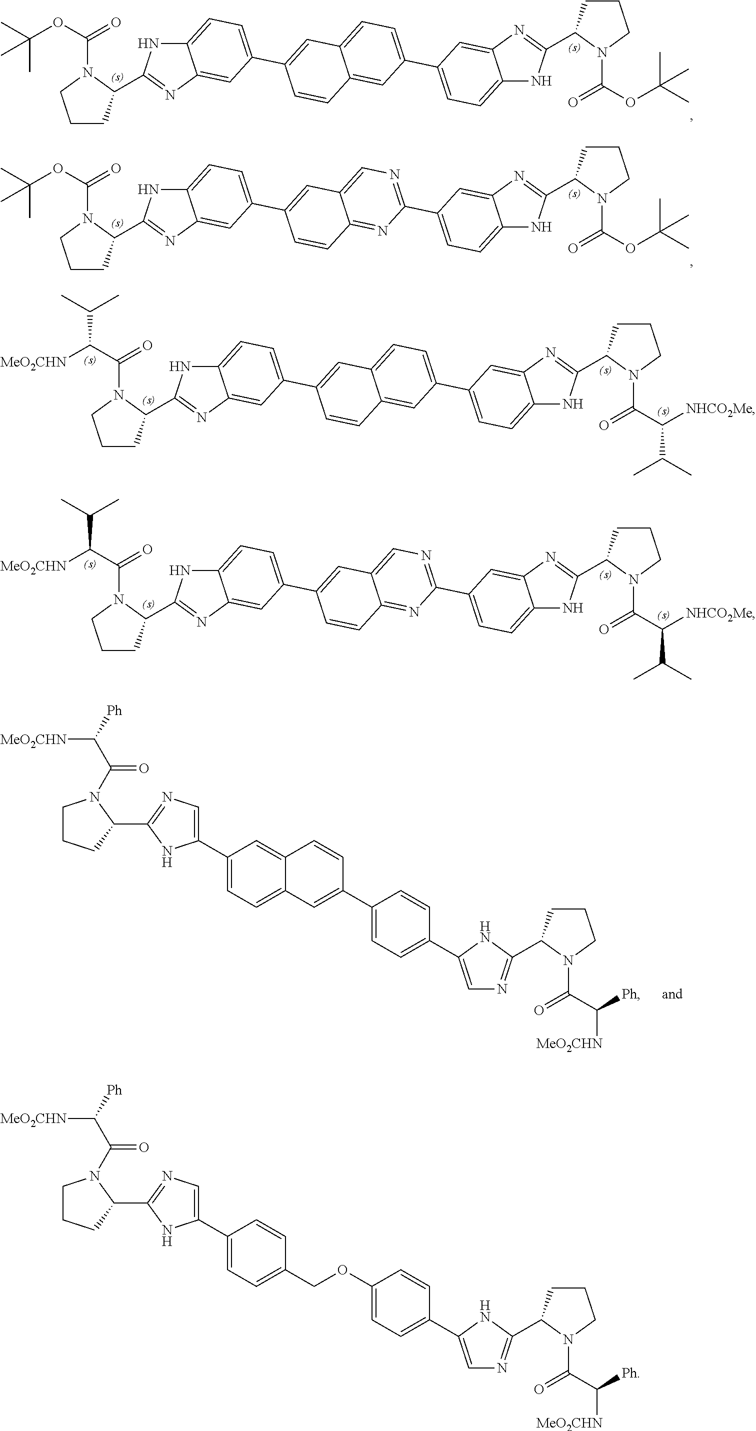

Some embodiments provide a pharmaceutical composition comprising a pharmaceutically acceptable excipient and a compound of Formulas I, II, III, IV, or V.

Some embodiments provide a method of treating HCV infection in an individual, the method comprising administering to the individual an effective amount of a compound of Formulas I, II, III, IV, or V or a pharmaceutical composition comprising a pharmaceutically acceptable excipient and a compound of Formulas I, II, III, IV, or V.

Some embodiments provide a method of treating HCV infection in an individual, the method comprising administering to the individual an effective amount of a compound of Formulas I, II, III, IV, or V or a pharmaceutical composition comprising a pharmaceutically acceptable excipient and a compound of Formulas I, II, III, IV, or V. In some embodiments, the method further comprises identifying a subject suffering from a hepatitis C infection.

Some embodiments provide a method of treating liver fibrosis in an individual, the method comprising administering to the individual an effective amount of a compound of Formulas I, II, III, IV, or V or a pharmaceutical composition comprising a pharmaceutically acceptable excipient and a compound of Formulas I, II, III, IV, or V. In some embodiments, the method further comprises identifying a subject suffering from a hepatitis C infection.

Some embodiments provide a method of increasing liver function in an individual having a hepatitis C virus infection, the method comprising administering to the individual an effective amount of a compound of Formulas I, II, III, IV, or V or a pharmaceutical composition comprising a pharmaceutically acceptable excipient and a compound of Formulas I, II, III, IV, or V. In some embodiments, the method further comprises identifying a subject suffering from a hepatitis C infection.

### DETAILED DESCRIPTION OF THE PREFERRED EMBODIMENT

### Definitions

As used herein, common organic abbreviations are defined as follows:
Ac Acetyl
Ac₂O Acetic anhydride
aq. Aqueous
Bn Benzyl
Bz Benzoyl
BOC or Boc tert-Butoxycarbonyl
Bu n-Butyl
cat. Catalytic
Cbz Carbobenzyloxy
CDI 1,1′-carbonyldiimidazole
Cy (c-C₆H₁₁ Cyclohexyl
° C. Temperature in degrees Centigrade
DBU 1,8-Diazabicyclo[5.4.0]undec-7-ene
DCE 1,2-Dichloroethane
DCM methylene chloride
DIEA Diisopropylethylamine
DMA Dimethylacetamide
DME Dimethoxyethane
DMF N,N′-Dimethylformamide
DMSO Dimethylsulfoxide
Et Ethyl
EtOAc Ethyl acetate
g Gram(s)
h Hour (hours)
HATU 2-(1H-7-azabenzotriazol-1-yl)-1,1,3,3-tetramethyl uronium hexafluorophosphate
HOBT N-Hydroxybenzotriazole
iPr Isopropyl
LCMS Liquid chromatography-mass spectrometry
LDA Lithium diisopropylamide
mCPBA meta-Chloroperoxybenzoic Acid
MeOH Methanol
MeCN Acetonitrile
mL Milliliter(s)
MTBE Methyl tertiary-butyl ether
NH₄OAc Ammonium acetate
PG Protecting group
Pd/C Palladium on activated carbon
Ph Phenyl
ppt Precipitate
RCM Ring closing metathesis
rt Room temperature
sBuLi sec-Butylithium
TEA Triethylamine
TCDI 1,1′-Thiocarbonyl diimidazole
Tert, t tertiary
TFA Trifluoracetic acid
THF Tetrahydrofuran
TLC Thin-layer chromatography
TMEDA Tetramethylethylenediamine
μL Microliter(s)

The terms “individual,” “host,” “subject,” and “patient” are used interchangeably herein, and refer to a mammal, including, but not limited to, primates, including simians and humans.

As used herein, the term “liver function” refers to a normal function of the liver, including, but not limited to, a synthetic function, including, but not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

The term “sustained viral response” (SVR; also referred to as a “sustained response” or a “durable response”), as used herein, refers to the response of an individual to a treatment regimen for HCV infection, in terms of serum HCV titer. Generally, a “sustained viral response” refers to no detectable HCV RNA (e.g., less than about 500, less than about 200, or less than about 100 genome copies per milliliter serum) found in the patient's serum for a period of at least about one month, at least about two months, at least about three months, at least about four months, at least about five months, or at least about six months following cessation of treatment.

“Treatment failure patients” as used herein generally refers to HCV-infected patients who failed to respond to previous therapy for HCV (referred to as “non-responders”) or who initially responded to previous therapy, but in whom the therapeutic response was not maintained (referred to as “relapsers”). The previous therapy generally can include treatment with IFN-α monotherapy or IFN-α combination therapy, where the combination therapy may include administration of IFN-α and an antiviral agent such as ribavirin.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

As used herein, the term “alkyl” refers to a branched or unbranched fully saturated acyclic aliphatic hydrocarbon group (i.e. composed of carbon and hydrogen containing no double or triple bonds). In some embodiments, alkyls may be substituted or unsubstituted. Alkyls include, but are not limited to, methyl, ethyl, propyl, isopropyl, butyl, isobutyl, tertiary butyl, pentyl, hexyl, and the like, each of which may be optionally substituted in some embodiments.

As used herein, the term “heteroalkyl” refers to a branched or unbrached fully saturated acyclic aliphatic hydrocarbon group containing one or more heteroatoms in the carbon back bone (i.e., an alkyl group in which one or more carbon atoms is replaced with a heteroatom). In some embodiments, heteroalkyls may be substituted or unsubstituted. Heteroalkyls include, but are not limited to, ethers, thioethers, and alkyl-amino-alkyls.

The term “halo” used herein refers to fluoro, chloro, bromo, or iodo.

The term “alkoxy” used herein refers to straight or branched chain alkyl radical covalently bonded to the parent molecule through an —O— linkage. In some embodiments, alkoxys may be substituted or unsubstituted. Examples of alkoxy groups include, but are not limited to, methoxy, ethoxy, propoxy, isopropoxy, butoxy, n-butoxy, sec-butoxy, t-butoxy and the like.

The term “alkenyl” used herein refers to a monovalent straight or branched chain radical of from two to twenty carbon atoms containing at least one carbon-carbon double bond including, but not limited to, 1-propenyl, 2-propenyl, 2-methyl-1-propenyl, 1-butenyl, 2-butenyl, and the like. In some embodiments, alkenyls may be substituted or unsubstituted.

The term “alkynyl” used herein refers to a monovalent straight or branched chain radical of from two to twenty carbon atoms containing at least one carbon-carbon triple bond including, but not limited to, 1-propynyl, 1-butynyl, 2-butynyl, and the like. In some embodiments, alkynyls may be substituted or unsubstituted.

The term “aryl” used herein refers to homocyclic aromatic radical having one ring or multiple fused rings. Examples of aryl groups include, but are not limited to, phenyl, naphthyl, biphenyl, phenanthrenyl, naphthacenyl, and the like. In some embodiments, aryls may be substituted or unsubstituted.

The term “cycloalkyl” used herein refers to saturated aliphatic ring system radical having three to twenty carbon atoms including, but not limited to, cyclopropyl, cyclopentyl, cyclohexyl, cycloheptyl, and the like. In some embodiments, cycloalkyls may be substituted or unsubstituted.

The term “cycloalkenyl” used herein refers to aliphatic ring system radical having three to twenty carbon atoms having at least one carbon-carbon double bond in the ring. Examples of cycloalkenyl groups include, but are not limited to, cyclopropenyl, cyclopentenyl, cyclohexenyl; cycloheptenyl, and the like. In some embodiments, cycloalkenyls may be substituted or unsubstituted.

The term “heterocyclic” or “heterocyclyl” or “heterocycloalkyl” used herein refers to cyclic ring system radical having at least one non-aromatic ring in which one or more ring atoms are not carbon, namely heteroatom. Monocyclic “heterocyclic” or “heterocyclyl” moieties are non-aromatic. Bicyclic “heterocyclic” or “heterocyclyl” moieties include one non-aromatic ring wherein at least one heteroatom is present in the non-aromatic ring. Examples of heterocyclic groups include, but are not limited to, morpholinyl, tetrahydrofuranyl, dioxolanyl, pyrrolidinyl, oxazolyl, pyranyl, pyrrolyl, isoindoline and the like.

The term “heteroaryl” used herein refers to an aromatic ring system radical in which one or more ring atoms are not carbon, namely heteroatom, having one ring or multiple fused rings. In fused ring systems, the one or more heteroatoms may be present in only one of the rings. Examples of heteroaryl groups include, but are not limited to, benzothiazyl, benzoxazyl, quinazolinyl, quinolinyl, isoquinolinyl, quinoxalinyl, pyridinyl, pyrrolyl, oxazolyl, indolyl, and the like.

The term “heteroatom” used herein refers to, for example, oxygen, sulfur and nitrogen.

The term “arylalkyl” used herein refers to one or more aryl groups appended to an alkyl radical. Examples of arylalkyl groups include, but are not limited to, benzyl, phenethyl, phenpropyl, phenbutyl, and the like.

The term “cycloalkylalkyl” used herein refers to one or more cycloalkyl groups appended to an alkyl radical. Examples of cycloalkylalkyl include, but are not limited to, cyclohexylmethyl, cyclohexylethyl, cyclopentylmethyl, cyclopentylethyl, and the like. In some embodiments, cycloalkylalkyls may be substituted or unsubstituted.

The term “heteroarylalkyl” used herein refers to one or more heteroaryl groups appended to an alkyl radical. Examples of heteroarylalkyl include, but are not limited to, pyridylmethyl, furanylmethyl, thiopheneylethyl, and the like. In some embodiments, heteroarylalkyls may be substituted or unsubstituted, and can be substituted on either the heteroaryl or alkyl portion or on both.

The term “heterocyclylalkyl” used herein refers to one or more heterocyclyl groups appended to an alkyl radical. Examples of heterocyclylalkyl include, but are not limited to, morpholinylmethyl, morpholinylethyl, morpholinylpropyl, tetrahydrofuranylmethyl, pyrrolidinylpropyl, and the like. In some embodiments, heterocyclylalkyls may be substituted or unsubstituted, and can be substituted on either the heterocyclyl or alkyl portion or on both.

The term “aryloxy” used herein refers to an aryl radical covalently bonded to the parent molecule through an —O— linkage.

The term “alkylthio” used herein refers to straight or branched chain alkyl radical covalently bonded to the parent molecule through an —S— linkage. Examples of alkylthio groups include, but are not limited to, methanesulfide, ethanesulfide, propanesulfide, isopropanesulfide, butanesulfide, n-butanesulfide, sec-butanesulfide, tert-butanesulfide and the like.

The term “arylthio” used herein refers to an aryl radical covalently bonded to the parent molecule through an —S— linkage.

The term “alkylamino” used herein refers to nitrogen radical with one or more alkyl groups attached thereto. Thus, monoalkylamino refers to nitrogen radical with one alkyl group attached thereto and dialkylamino refers to nitrogen radical with two alkyl groups attached thereto.

The term “cyanoamino” used herein refers to nitrogen radical with nitrile group attached thereto.

The term “carbamyl” used herein refers to RNHCOO—.

The term “keto” and “carbonyl” used herein refers to C═O.

The term “carboxy” used herein refers to —COOH.

The term “sulfamyl” used herein refers to —SO₂NH₂.

The term “sulfonyl” used herein refers to —SO₂—.

The term “sulfinyl” used herein refers to —SO—.

The term “thiocarbonyl” used herein refers to C═S.

The term “thiocarboxy” used herein refers to CSOH.

The term “sulfonamide” used herein refers to —SO₂NR′₂ where each R′ is individually selected from H (hydrogen), C₁-C₆ alkyl, C₃-C₇ cycloalkyl, arylalkyl and aryl optionally substituted with C₁-C₆ alkyl.

The term “ester” used herein refers to —COOR′ where R′ is selected from C₁-C₆ alkyl, C₃-C₇ cycloalkyl, arylalkyl and aryl optionally substituted with C₁-C₆ alkyl.

The term “C-amide” used herein refers to —C(═O)NR′₂ where each R′ is individually selected from H (hydrogen), C₁-C₆ alkyl, C₃-C₇ cycloalkyl, arylalkyl and aryl optionally substituted with C₁-C₆ alkyl.

The term “N-amide” used herein refers to —NR′C(═O)R′ where each R′ is individually selected from H (hydrogen), C₁-C₆ alkyl, C₃-C₇ cycloalkyl, arylalkyl and aryl optionally substituted with C₁-C₆ alkyl.

The term “N-carbamate” used herein refers to —NR′C(═O)OR′ where each R′ is individually selected from H (hydrogen), C₁-C₆ alkyl, C₃-C₇ cycloalkyl, arylalkyl and aryl optionally substituted with C₁-C₆ alkyl.

The term “O-carbamate” used herein refers to —OC(═O)NR′₂ where each R′ is individually selected from H (hydrogen), C₁-C₆ alkyl, C₃-C₇ cycloalkyl, arylalkyl and aryl optionally substituted with C₁-C₆ alkyl.

The term “urea” used herein refers to —NR′C(═O)NR′₂ where each R′ is individually selected from H (hydrogen), C₁-C₆ alkyl, C₃-C₇ cycloalkyl, arylalkyl and aryl optionally substituted with C₁-C₆ alkyl.

As used herein, a radical indicates a species with one or more, unpaired electron such that the species containing the radical can be covalently bonded to one or more other species. Hence, in this context, a radical is not necessarily a free radical. Rather, a radical indicates a specific portion of a larger molecule. The term “radical” can be used interchangeably with the term “moiety” or “group.”

As used herein, a substituted group is derived from the unsubstituted parent structure in which there has been an exchange of one or more hydrogen atoms for another atom or group. When substituted, the substituent group(s) is (are) one or more group(s) individually and independently selected from C₁-C₆ alkyl, C₁-C₆ alkenyl, C₁-C₆ alkynyl, C₃-C₇ cycloalkyl (optionally substituted with halo, alkyl, alkoxy, carboxyl, haloalkyl, CN, —SO₂-alkyl, —CF₃, and —OCF₃), cycloalkyl geminally attached, C₁-C₆ heteroalkyl, C₃-C₁₀ heterocycloalkyl (e.g., tetrahydrofuryl) (optionally substituted with halo, alkyl, alkoxy, carboxyl, CN, —SO₂-alkyl, —CF₃, and —OCF₃), aryl (optionally substituted with halo, alkyl, aryl optionally substituted with C₁-C₆ alkyl, arylalkyl, alkoxy, carboxyl, CN, —SO₂-alkyl, —CF₃, and —OCF₃), arylalkyl (optionally substituted with halo, alkyl, alkoxy, aryl, carboxyl, CN, —SO₂-alkyl, —CF₃, and —OCF₃), heteroaryl (optionally substituted with halo, alkyl, alkoxy, aryl, aralkyl, carboxyl, CN, —SO₂-alkyl, —CF₃, and —OCF₃), halo (e.g., chloro, bromo, iodo and fluoro), cyano, hydroxy, —CF₃, C₁-C₆ alkoxy, aryloxy, sulfhydryl (mercapto), halo(C₁-C₆)alkyl, C₁-C₆ alkylthio, arylthio, mono- and di-(C₁-C₆)alkyl amino, quaternary ammonium salts, amino(C₁-C₆)alkoxy, hydroxy(C₁-C₆)alkylamino, amino(C₁-C₆)alkylthio, cyanoamino, nitro, carbamyl, keto (oxy), carbonyl, carboxy, glycolyl, glycyl, hydrazino, guanyl, sulfamyl, sulfonyl, sulfinyl, thiocarbonyl, thiocarboxy, sulfonamide, ester, C-amide, N-amide, N-carbamate, O-carbamate, urea and combinations thereof. The protecting groups that can form the protective derivatives of the above substituents are known to those of skill in the art and can be found in references such as Greene and Wuts Protective Groups in Organic Synthesis; John Wiley and Sons: New York, 1999. Wherever a substituent is described as “optionally substituted” that substituent can be substituted with the above substituents.

Asymmetric carbon atoms may be present in the compounds described. All such isomers, including diastereomers and enantiomers, as well as the mixtures thereof are intended to be included in the scope of the recited compound. In certain cases, compounds can exist in tautomeric forms. All tautomeric forms are intended to be included in the scope. Likewise, when compounds contain an alkenyl or alkenylene group, there exists the possibility of cis- and trans-isomeric forms of the compounds. Both cis- and trans-isomers, as well as the mixtures of cis- and trans-isomers, are contemplated. Thus, reference herein to a compound includes all of the aforementioned isomeric forms unless the context clearly dictates otherwise.

Various forms are included in the embodiments, including polymorphs, solvates, hydrates, conformers, salts, and prodrug derivatives. A polymorph is a composition having the same chemical formula, but a different structure. A solvate is a composition formed by solvation (the combination of solvent molecules with molecules or ions of the solute). A hydrate is a compound formed by an incorporation of water. A conformer is a structure that is a conformational isomer. Conformational isomerism is the phenomenon of molecules with the same structural formula but different conformations (conformers) of atoms about a rotating bond. Salts of compounds can be prepared by methods known to those skilled in the art. For example, salts of compounds can be prepared by reacting the appropriate base or acid with a stoichiometric equivalent of the compound. A prodrug is a compound that undergoes biotransformation (chemical conversion) before exhibiting its pharmacological effects. For example, a prodrug can thus be viewed as a drug containing specialized protective groups used in a transient manner to alter or to eliminate undesirable properties in the parent molecule. Thus, reference herein to a compound includes all of the aforementioned forms unless the context clearly dictates otherwise.

The term “pharmaceutically acceptable salt,” as used herein, and particularly when referring to a pharmaceutically acceptable salt of a compound, including a compound of Formulas I, II, III, IV, or V, as produced and synthesized by the methods disclosed herein, refers to any pharmaceutically acceptable salts of a compound, and preferably refers to an acid addition salt of a compound. With respect to compounds synthesized by the method of this embodiment that contain a basic nitrogen, the preferred examples of pharmaceutically acceptable salts are acid addition salts of pharmaceutically acceptable inorganic or organic acids, including but not limited to hydrohalic, sulfuric, phosphoric, or aliphatic or aromatic carboxylic, or sulfonic acid. Examples of pharmaceutically acceptable inorganic or organic acids as a component of an addition salt, include but are not limited to, hydrochloric acid, hydrobromic acid, phosphoric acid, sulfuric acid acetic acid, succinic acid, lactic acid, malic acid, tartaric acid, citric acid, ascorbic acid, nicotinic acid, methanesulfonic acid, p-toluenesulfonic acid or naphthalenesulfonic acid. With respect to compounds synthesized by the methods of this embodiment that contain an acidic functional group, the preferred examples of pharmaceutically acceptable salts include, but are not limited to, alkali metal salts (sodium or potassium), alkaline earth metal salts (calcium or magnesium), or ammonium salts derived from ammonia or from pharmaceutically acceptable organic amines, for example C₁-C₇ alkylamine, cyclohexylamine, triethanolamine, ethylenediamine or tris-(hydroxymethyl)-aminomethane.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Wherever a substituent as depicted as a di-radical (i.e., has two points of attachment to the rest of the molecule), it is to be understood that the substituent can be attached in any directional configuration unless otherwise indicated. Thus, for example, a substituent depicted as -AE- or

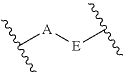

includes the substituent being oriented such that the A is attached at the leftmost attachment point of the molecule as well as the case in which A is attached at the rightmost attachment point of the molecule.

It is to be understood that certain radical naming conventions can include either a mono-radical or a di-radical, depending on the context. For example, where a substituent requires two points of attachment to the rest of the molecule, it is understood that the substituent is a di-radical. A substituent identified as alkyl, that requires two points of attachment, includes di-radicals such as —CH₂—, —CH₂CH₂—, —CH₂CH(CH₃)CH₂—, and the like; a substituent depicted as alkoxy that requires two points of attachment, includes di-radicals such as —OCH₂—, —OCH₂CH₂—, —OCH₂CH(CH₃)CH₂—, and the like: and a substituent depicted as arylC(═O)— that requires two points of attachment, includes di-radicals such as

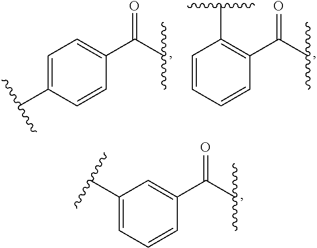

and the like.

Where a range of values is provided, it is understood that the upper and lower limit, and each intervening value between the upper and lower limit of the range is encompassed within the embodiments.

Unless defined otherwise, all technical and scientific terms used herein have the same meaning as commonly understood by one of ordinary skill in the art to which the embodiments belong. Although any methods and materials similar or equivalent to those described herein can also be used in the practice or testing of the embodiments, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

### Compounds

The present embodiments provide compounds of Formulas I, II, III, IV, or V, as defined above, as well as pharmaceutical compositions and formulations comprising any compound of Formulas I, II, III, IV, or V. A subject compound is useful for treating HCV infection and other disorders, as discussed below.

In many embodiments, a subject compound inhibits HCV viral replication. For example, a subject compound inhibits HCV viral replication by at least about 10%, at least about 15%, at least about 20%, at least about 25%, at least about 30%, at least about 40%, at least about 50%, at least about 60%, at least about 70%, at least about 80%, or at least about 90%, or more, compared to HCV viral replication in the absence of the compound. Whether a subject compound inhibits HCV viral replication can be determined using methods known in the art, including an in vitro viral replication assay.

### Compositions

The present embodiments further provide compositions, including pharmaceutical compositions, comprising compounds of the general Formulas I, II, III, IV, or V.

A subject pharmaceutical composition comprises a subject compound; and a pharmaceutically acceptable excipient. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are known in the art. Moreover, pharmaceutically acceptable auxiliary substances, such as pH adjusting and buffering agents, tonicity adjusting agents, stabilizers, wetting agents and the like, are known in the art.

In some embodiments, a compound as described herein can be formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

As such, administration of a compound as described herein can be achieved in various ways, including oral, buccal, rectal, parenteral, intraperitoneal, intradermal, subcutaneous, intramuscular, transdermal, intratracheal, etc., administration. In some embodiments, administration is by bolus injection, e.g., subcutaneous bolus injection, intramuscular bolus injection, and the like.

The pharmaceutical compositions of the embodiments can be administered orally, parenterally or via an implanted reservoir. Oral administration or administration by injection is preferred.

Subcutaneous administration of a pharmaceutical composition of the embodiments is accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of a pharmaceutical composition of the embodiments to a patient through the port is referred to herein as “a subcutaneous injection port delivery system.” In many embodiments, subcutaneous administration is achieved by bolus delivery by needle and syringe.

In pharmaceutical dosage forms, the compounds as described herein may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the compounds as described herein can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with, disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds as described herein can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the compounds as described herein can be made into suppositories by mixing with a variety of bases such as emulsifying bases or water-soluble bases. The compounds of the embodiments can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds as described herein. Similarly, unit dosage forms for injection or intravenous administration may comprise the compounds as described herein in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the embodiments calculated in an amount sufficient to produce the desired effect in association with a pharmaceutically acceptable diluent, carrier or vehicle. The specifications for the novel unit dosage forms of the embodiments depend on the particular compound employed and the effect to be achieved, and the pharmacodynamics associated with each compound in the host.

The pharmaceutically acceptable excipients, such as vehicles, adjuvants, carriers or diluents, are known in the art. Moreover, pharmaceutically acceptable auxiliary substances, such as pH adjusting and buffering agents, tonicity adjusting agents, stabilizers, wetting agents and the like, are known in the art.

### Treating a Hepatitis Virus Infection

The methods and compositions described herein are generally useful in treatment of an of HCV infection.

Preferred embodiments provide a method of treating a hepatitis C virus infection in an individual, the method comprising administering to the individual an effective amount of a composition comprising a subject compound.

Preferred embodiments provide a method of treating liver fibrosis in an individual, the method comprising administering to the individual an effective amount of a composition comprising a subject compound.

Preferred embodiments provide a method of increasing liver function in an individual having a hepatitis C virus infection, the method comprising administering to the individual an effective amount of a composition comprising a subject compound.

Whether a subject method is effective in treating an HCV infection can be determined by a reduction in viral load, a reduction in time to seroconversion (virus undetectable in patient serum), an increase in the rate of sustained viral response to therapy, a reduction of morbidity or mortality in clinical outcomes, or other indicator of disease response.

In general, an effective amount of a compound of Formulas I, II, III, IV, or V, and optionally one or more additional antiviral agents, is an amount that is effective to reduce viral load or achieve a sustained viral response to therapy.

Whether a subject method is effective in treating an HCV infection can be determined by measuring viral load, or by measuring a parameter associated with HCV infection, including, but not limited to, liver fibrosis, elevations in serum transaminase levels, and necroinflammatory activity in the liver. Indicators of liver fibrosis are discussed in detail below.

In some embodiments, the methods involve administering an effective amount of a compound of Formulas I, II, III, IV, or V, optionally in combination with an effective amount of one or more additional antiviral agents. In some embodiments, an effective amount of a compound of Formulas I, II, III, IV, or V, and optionally one or more additional antiviral agents, is an amount that is effective to reduce viral titers to undetectable levels, e.g., to about 1000 to about 5000, to about 500 to about 1000, or to about 100 to about 500 genome copies/mL serum. In some embodiments, an effective amount of a compound of Formulas I, II, III, IV, or V, and optionally one or more additional antiviral agents, is an amount that is effective to reduce viral load to lower than 100 genome copies/mL serum.

In some embodiments, an effective amount of a compound of Formulas I, II, III, IV, or V, and optionally one or more additional antiviral agents, is an amount that is effective to achieve a 1.5-log, a 2-log, a 2.5-log, a 3-log, a 3.5-log, a 4-log, a 4.5-log, or a 5-log reduction in viral titer in the serum of the individual.

In many embodiments, an effective amount of a compound of Formulas I, II, III, IV, or V, and optionally one or more additional antiviral agents, is an amount that is effective to achieve a sustained viral response, e.g., non-detectable or substantially non-detectable HCV RNA (e.g., less than about 500, less than about 400, less than about 200, or less than about 100 genome copies per milliliter serum) is found in the patient's serum for a period of at least about one month, at least about two months, at least about three months, at least about four months, at least about five months, or at least about six months following cessation of therapy.

As noted above, whether a subject method is effective in treating an HCV infection can be determined by measuring a parameter associated with HCV infection, such as liver fibrosis. Methods of determining the extent of liver fibrosis are discussed in detail below. In some embodiments, the level of a serum marker of liver fibrosis indicates the degree of liver fibrosis.

As one non-limiting example, levels of serum alanine aminotransferase (ALT) are measured, using standard assays. In general, an ALT level of less than about 45 international units is considered normal. In some embodiments, an effective amount of a compound of Formulas I, II, III, IV, or V, and optionally one or more additional antiviral agents, is an amount effective to reduce ALT levels to less than about 45 IU/mL serum.

A therapeutically effective amount of a compound of Formulas I, IV, or V, and optionally one or more additional antiviral agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Methods of measuring serum markers include immunological-based methods, e.g., enzyme-linked immunosorbent assays (ELISA), radioimmunoassays, and the like, using antibody specific for a given serum marker.

In many embodiments, an effective amount of a compound of Formulas I, II, III, IV, or V and an additional antiviral agent is a synergistic amount. As used herein, a “synergistic combination” or a “synergistic amount” of a compound of Formulas I, II, III, IV, or V and an additional antiviral agent is a combined dosage that is more effective in the therapeutic or prophylactic treatment of an HCV infection than the incremental improvement in treatment outcome that could be predicted or expected from a merely additive combination of (i) the therapeutic or prophylactic benefit of the compound of Formulas I, II, III, IV, or V when administered at that same dosage as a monotherapy and (ii) the therapeutic or prophylactic benefit of the additional antiviral agent when administered at the same dosage as a monotherapy.

In some embodiments, a selected amount of a compound of Formulas I, II, III, IV, or V and a selected amount of an additional antiviral agent are effective when used in combination therapy for a disease, but the selected amount of the compound of Formulas I, II, III, IV, or V and/or the selected amount of the additional antiviral agent is less effective when used in monotherapy for the disease. Thus, the embodiments encompass (1) regimens in which a selected amount of the additional antiviral agent enhances the therapeutic benefit of a selected amount of the compound of Formulas I, II, III, IV, or V when used in combination therapy for a disease, where the selected amount of the additional antiviral agent provides less therapeutic benefit when used in monotherapy for the disease (2) regimens in which a selected amount of the compound of Formulas I, II, III, IV, or V enhances the therapeutic benefit of a selected amount of the additional antiviral agent when used in combination therapy for a disease, where the selected amount of the compound of Formulas I, II, III, IV, or V provides less therapeutic benefit when used in monotherapy for the disease and (3) regimens in which a selected amount of the compound of Formulas I, II, III, IV, or V and a selected amount of the additional antiviral agent provide a therapeutic benefit when used in combination therapy for a disease, where each of the selected amounts of the compound of Formulas I, II, III, IV, or V and the additional antiviral agent, respectively, provides less therapeutic benefit when used in monotherapy for the disease. As used herein, a “synergistically effective amount” of a compound of Formulas I, II, III, IV, or V and an additional antiviral agent, and its grammatical equivalents, shall be understood to include any regimen encompassed by any of (1)-(3) above.

### Fibrosis

The embodiments provides methods for treating liver fibrosis (including forms of liver fibrosis resulting from, or associated with, HCV infection), generally involving administering a therapeutic amount of a compound of Formulas I, II, III, IV, or V, and optionally one or more additional antiviral agents. Effective amounts of compounds of Formulas I, II, III, IV, or V, with and without one or more additional antiviral agents, as well as dosing regimens, are as discussed below.

Whether treatment with a compound of Formulas I, II, III, IV, or V, and optionally one or more additional antiviral agents, is effective in reducing liver fibrosis is determined by any of a number of well-established techniques for measuring liver fibrosis and liver function. Liver fibrosis reduction can be determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

The Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite.

The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

In some embodiments, a therapeutically effective amount of a compound of Formulas I, II, III, IV, or V, and optionally one or more additional antiviral agents, is an amount that effects a change of one unit or more in the fibrosis stage based on pre- and post-therapy liver biopsies. In particular embodiments, a therapeutically effective amount of a compound of Formulas I, II, III, IV, or V, and optionally one or more additional antiviral agents, reduces liver fibrosis by at least one unit in the METAVIR, the Knodell, the Scheuer, the Ludwig, or the Ishak scoring system.

Secondary, or indirect, indices of liver function can also be used to evaluate the efficacy of treatment with a compound of Formulas I, II, III, IV, or V. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score.

An effective amount of a compound of Formulas I, II, III, IV, or V, and optionally one or more additional antiviral agents, is an amount that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or to a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

A therapeutically effective amount of a compound of Formulas I, II, III, IV, or V, and optionally one or more additional antiviral agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

A therapeutically effective amount of a compound of Formulas I, II, III, IV, or V, and optionally one or more additional antiviral agents, is an amount that is effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or to a placebo-treated individual.

Whether treatment with a compound of Formulas I, II, III, IV, or V, and optionally one or more additional antiviral agents, is effective in reducing the incidence of a disorder associated with cirrhosis of the liver can readily be determined by those skilled in the art.

Reduction in liver fibrosis can increase liver function. Thus, the embodiments provide methods for increasing liver function, generally involving administering a therapeutically effective amount of a compound of Formulas I, II, III, IV, or V, and optionally one or more additional antiviral agents. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal level of alanine transaminase is about 45 IU per milliliter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

A therapeutically effective amount of a compound of Formulas I, II, III, IV, or V, and optionally one or more additional antiviral agents, is one that is effective to increase liver function by at least about 10%, at least about 20%, at least about 30%, at least about 40%, at least about 50%, at least about 60%, at least about 70%, at least about 80%, or more. For example, a therapeutically effective amount of a compound of Formulas I, II, III, IV, or V, and optionally one or more additional antiviral agents, is an amount effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of a compound of Formulas I, II, III, IV, or V, and optionally one or more additional antiviral agents, is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

### Dosages, Formulations, and Routes of Administration

In the subject methods, the active agent(s) (e.g., compound of Formulas I, II, III, IV, or V, and optionally one or more additional antiviral agents) may be administered to the host using any convenient means capable of resulting in the desired therapeutic effect. Thus, the agent can be incorporated into a variety of formulations for therapeutic administration. More particularly, the agents of the embodiments can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

### Other Antiviral or Antifibrotic Agents

As discussed above, a subject method will in some embodiments be carried out by administering a compound of Formulas I, II, III, IV, or V, and optionally one or more additional antiviral agent(s).

In some embodiments, the method further includes administration of one or more interferon receptor agonist(s).

In other embodiments, the method further includes administration of pirfenidone or a pirfenidone analog.

Additional antiviral agents that are suitable for use in combination therapy include, but are not limited to, nucleotide and nucleoside analogs. Non-limiting examples include azidothymidine (AZT) (zidovudine), and analogs and derivatives thereof; 2′,3′-dideoxyinosine (DDI) (didanosine), and analogs and derivatives thereof; 2′,3′-dideoxycytidine (DDC) (dideoxycytidine), and analogs and derivatives thereof; 2′,3′-didehydro-2′,3′-dideoxythymidine (D4T) (stavudine), and analogs and derivatives thereof; combivir; abacavir; adefovir dipoxil; cidofovir; ribavirin; ribavirin analogs; and the like.

In some embodiments, the method further includes administration of ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. Some embodiments also involve use of derivatives of ribavirin (see, e.g., U.S. Pat. No. 6,277,830). The ribavirin may be administered orally in capsule or tablet form, or in the same or different administration form and in the same or different route as the subject compound. Of course, other types of administration of both medicaments, as they become available are contemplated, such as by nasal spray, transdermally, intravenously, by suppository, by sustained release dosage form, etc. Any form of administration will work so long as the proper dosages are delivered without destroying the active ingredient.

In some embodiments, the method further includes administration of ritonavir. Ritonavir, 10-hydroxy-2-methyl-5-(1-methylethyl)-1-[2-(1-methylethyl)-4-thiazolyl]-3,6-dioxo-8,11-bis(phenylmethyl)-2,4,7,12-tetraazatridecan-13-oic acid, 5-thiazolylmethyl ester [5S-(5R*,8R*,10R*,11R*)], available from Abbott Laboratories, is an inhibitor of the protease of the human immunodeficiency virus and also of the cytochrome P450 3A and P450 2D6 liver enzymes frequently involved in hepatic metabolism of therapeutic molecules in man.

In some embodiments, the method further includes administration of a protease inhibitor. In some embodiments, the method further includes administration of an NS5A inhibitor. In some embodiments, the method further includes administration of a helicase inhibitor. In some embodiments, the method further includes administration of a polymerase inhibitor.

In some embodiments, an additional antiviral agent is administered during the entire course of the subject compound treatment. In other embodiments, an additional antiviral agent is administered for a period of time that is overlapping with that of the subject compound treatment, e.g., the additional antiviral agent treatment can begin before the subject compound treatment begins and end before the subject compound treatment ends; the additional antiviral agent treatment can begin after the subject compound treatment begins and end after the subject compound treatment ends; the additional antiviral agent treatment can begin after the subject compound treatment begins and end before the subject compound treatment ends; or the additional antiviral agent treatment can begin before the subject compound treatment begins and end after the subject compound treatment ends.

### Methods of Treatment

### Monotherapies

The compounds as described herein may be used in acute or chronic therapy for HCV disease. In many embodiments, the compounds as described herein can be administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The compounds as described herein can be administered 5 times per day, 4 times per day, tid, bid, qd, qod, biw, tiw, qw, qow, three times per month, or once monthly. In other embodiments, the compounds as described herein can be administered as a continuous infusion.

In many embodiments, a compound described herein of the embodiments can be administered orally.

In connection with the above-described methods for the treatment of HCV disease in a patient, a compound as described herein may be administered to the patient at a dosage from about 0.01 mg to about 100 mg/kg patient bodyweight per day, in 1 to 5 divided doses per day. In some embodiments, a compound as described herein can be administered at a dosage of about 0.5 mg to about 75 mg/kg patient bodyweight per day, in 1 to 5 divided doses per day.

The amount of active ingredient that may be combined with carrier materials to produce a dosage form can vary depending on the host to be treated and the particular mode of administration. A typical pharmaceutical preparation can contain from about 5% to about 95% active ingredient (w/w). In other embodiments, the pharmaceutical preparation can contain from about 20% to about 80% active ingredient.

Those of skill will readily appreciate that dose levels can vary as a function of the specific compound, the severity of the symptoms and the susceptibility of the subject to side effects. Preferred dosages for a given compound are readily determinable by those of skill in the art by a variety of means. A preferred means can be to measure the physiological potency of a given interferon receptor agonist.

In many embodiments, multiple doses of a compound as described herein can be administered to a subject. For example, a compound as described herein can be administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Combination Therapies with a TNF-α Antagonist and an Interferon

Some embodiments provide a method of treating an HCV infection in an individual having an HCV infection, the method comprising administering an effective amount of a compound as described herein, and effective amount of a TNF-α antagonist, and an effective amount of one or more interferons.

### Subjects Suitable for Treatment

In certain embodiments, the specific regimen of drug therapy used in treatment of the HCV patient is selected according to certain disease parameters exhibited by the patient, such as the initial viral load, genotype of the HCV infection in the patient, liver histology and/or stage of liver fibrosis in the patient.

Any of the above treatment regimens can be administered to individuals who have been diagnosed with an HCV infection. Any of the above treatment regimens can be administered to individuals having advanced or severe stage liver fibrosis as measured by a Knodell score of 3 or 4 or no or early stage liver fibrosis as measured by a Knodell score of 0, 1, or 2. Any of the above treatment regimens can be administered to individuals who have failed previous treatment for HCV infection (“treatment failure patients,” including non-responders and relapsers).

Individuals who have been clinically diagnosed as infected with HCV are of particular interest in many embodiments. Individuals who are infected with HCV are identified as having HCV RNA in their blood, and/or having anti-HCV antibody in their serum. Such individuals include anti-HCV ELISA-positive individuals, and individuals with a positive recombinant immunoblot assay (RIBA). Such individuals may also, but need not, have elevated serum ALT levels.

Individuals who are clinically diagnosed as infected with HCV include naïve individuals (e.g., individuals not previously treated for HCV, particularly those who have not previously received IFN-α-based and/or ribavirin-based therapy) and individuals who have failed prior treatment for HCV (“treatment failure” patients). Treatment failure patients include non-responders (i.e., individuals in whom the HCV titer was not significantly or sufficiently reduced by a previous treatment for HCV, e.g., a previous IFN-α monotherapy, a previous IFN-α and ribavirin combination therapy, or a previous pegylated IFN-α and ribavirin combination therapy); and relapsers (i.e., individuals who were previously treated for HCV, e.g., who received a previous IFN-α monotherapy, a previous IFN-α and ribavirin combination therapy, or a previous pegylated IFN-α and ribavirin combination therapy, whose HCV titer decreased, and subsequently increased).

In particular embodiments of interest, individuals have an HCV titer of at least about 10⁵, at least about 5×10⁵, or at least about 10⁶, or at least about 2×10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1 and particular HCV subtypes and quasispecies.

Also of interest are HCV-positive individuals (as described above) who exhibit severe fibrosis or early cirrhosis (non-decompensated, Child's-Pugh class A or less), or more advanced cirrhosis (decompensated, Child's-Pugh class B or C) due to chronic HCV infection and who are viremic despite prior anti-viral treatment with IFN-α-based therapies or who cannot tolerate IFN-α-based therapies, or who have a contraindication to such therapies. In particular embodiments of interest, HCV-positive individuals with stage 3 or 4 liver fibrosis according to the METAVIR scoring system are suitable for treatment with the methods described herein. In other embodiments, individuals suitable for treatment with the methods of the embodiments are patients with decompensated cirrhosis with clinical manifestations, including patients with far-advanced liver cirrhosis, including those awaiting liver transplantation. In still other embodiments, individuals suitable for treatment with the methods described herein include patients with milder degrees of fibrosis including those with early fibrosis (stages 1 and 2 in the METAVIR, Ludwig, and Scheuer scoring systems; or stages 1, 2, or 3 in the Ishak scoring system.).

### Synthesis

The compounds and processes of the present disclosure will be better understood in connection with the following synthetic schemes which illustrate the methods by which the compounds of the present disclosure may be prepared. Starting materials can be obtained from commercial sources or prepared by well-established literature methods known to those of ordinary skill in the art. The variables are as defined above unless otherwise noted below.

### Section I

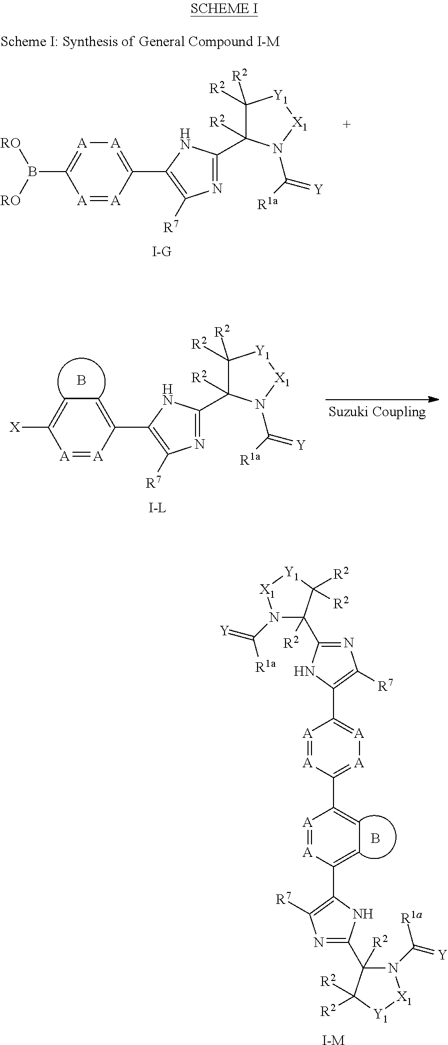

General compound I-G and general compound I-L can be coupled according to Scheme I to afford general compound I-M using standard Suzuki type coupling conditions (e.g., Angew Chem. Int. Ed. Engl 2001, 40, 4544). Intermediates I-G and I-L can be made according to Schemes I-A and I-B, respectively.

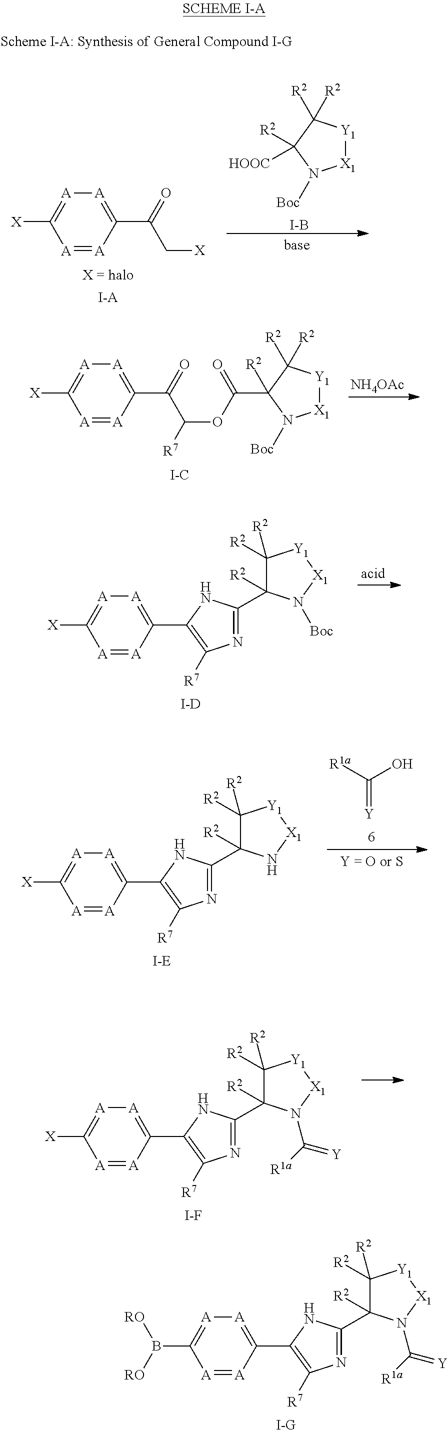

In some embodiments, the base used when converting I-A to I-C is DIEA in THF. In some embodiments, the step converting I-C to I-D is conducted in toluene. In some embodiments, the acid used in the step converting I-D to I-E is HCl in methanol. In some embodiments, the carboxylic acid used in the step converting I-E to I-F is

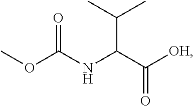

which may be formed according to the following reaction:

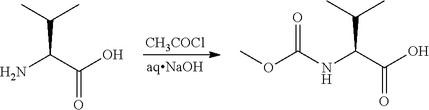

In some embodiments, compound I-G has the structure:

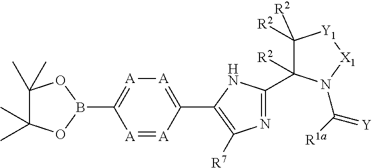

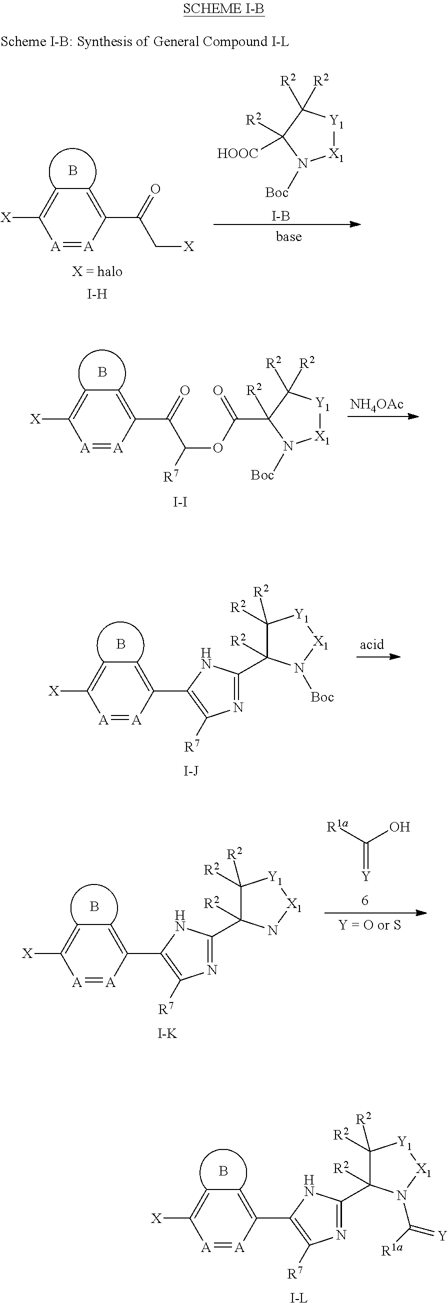

Intermediate I-H of the benzothiophene type can be synthesized according to Scheme I-C.

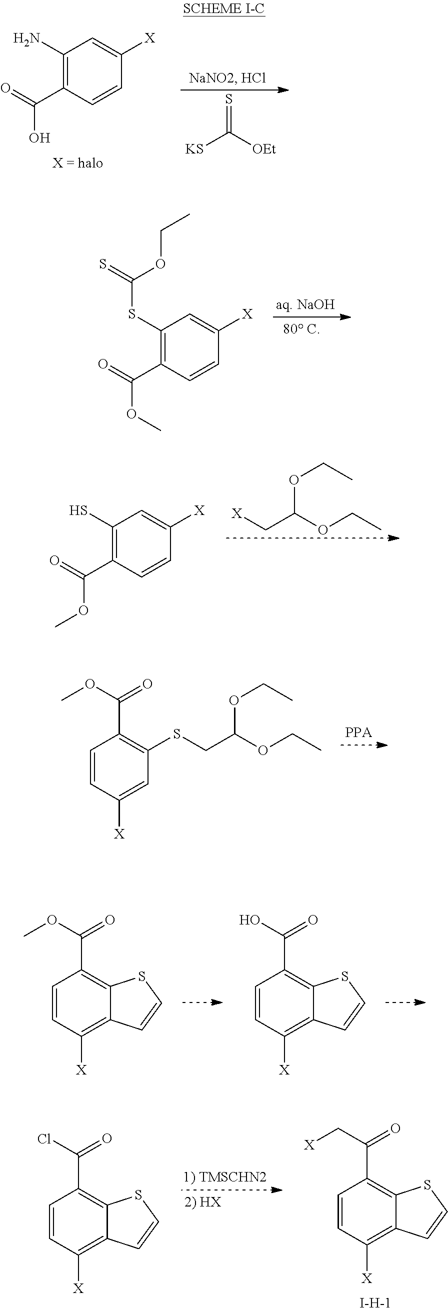

Intermediate I-H of the indole type can be synthesized according to Scheme I-D.

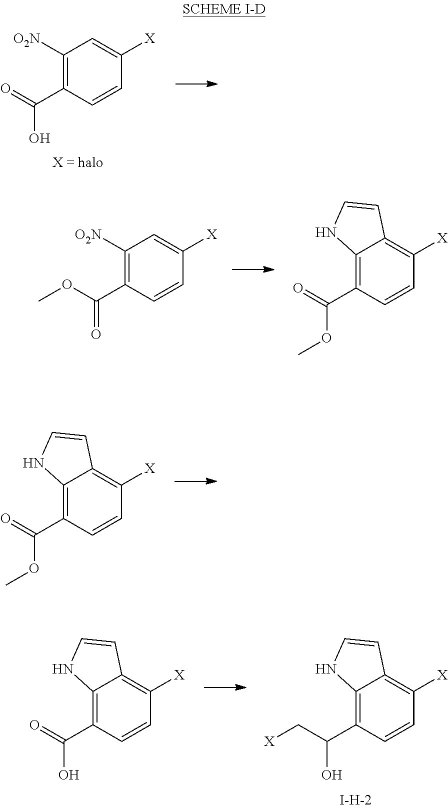

Intermediate I-H of the benzoimidazole type can be synthesized according to Scheme I-E.

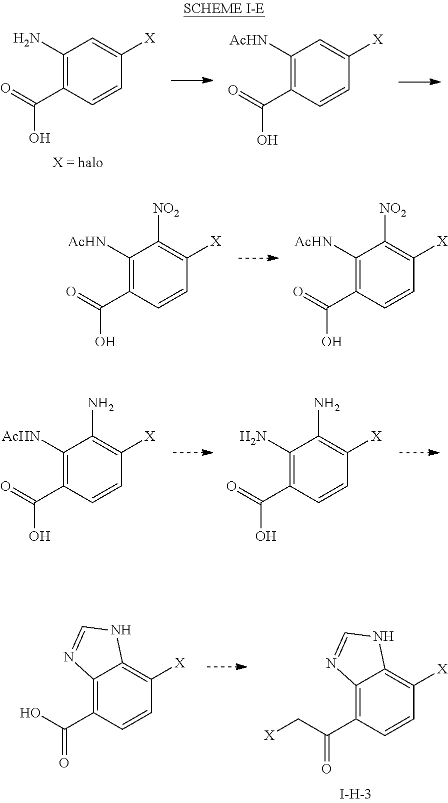

The compounds shown below in Table I can be prepared by the methods disclosed in Section I modified as appropriate. It will be readily apparent to one of ordinary skill in the art that the compounds shown below in Table I can be synthesized by use of the appropriate reactants, reagents and reaction conditions.

TABLE I

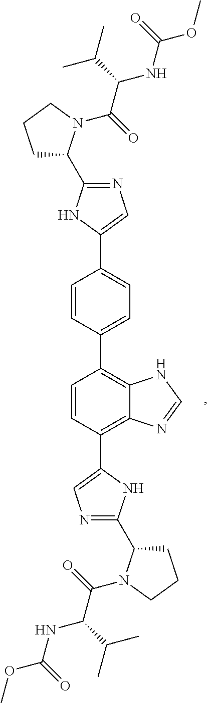

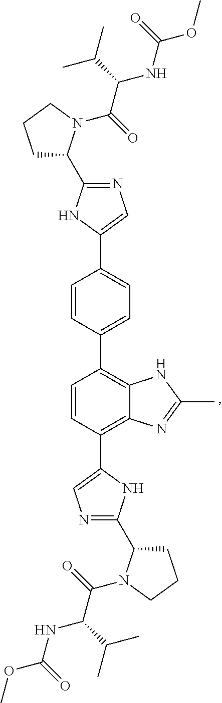

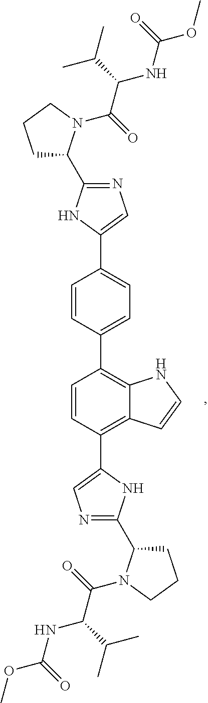

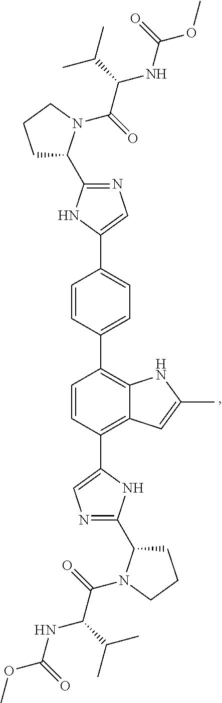

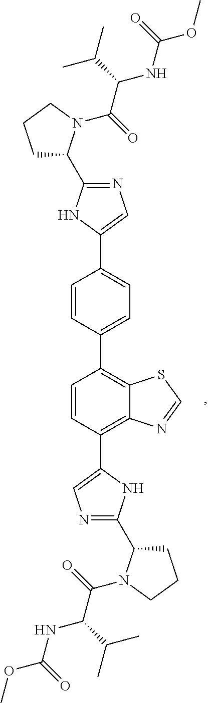

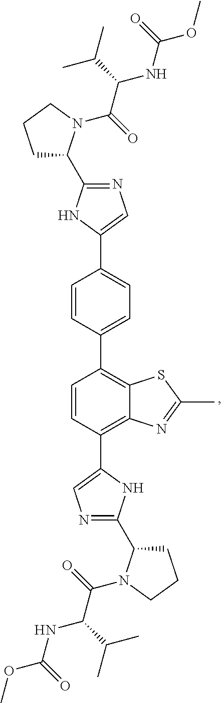

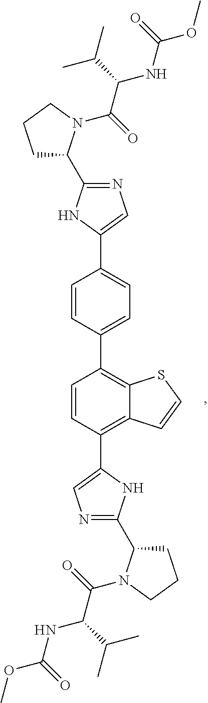

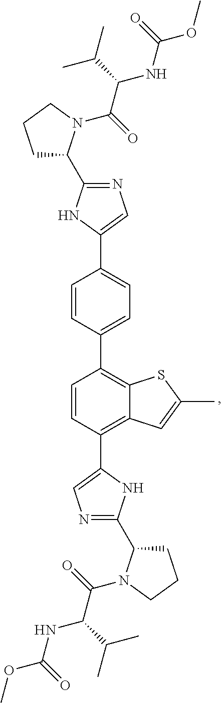

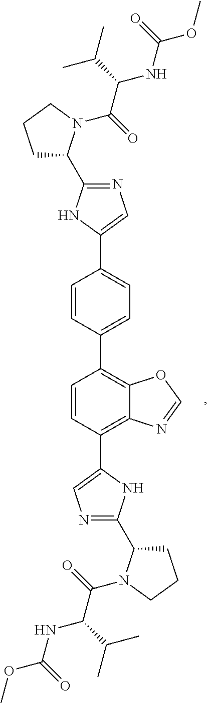

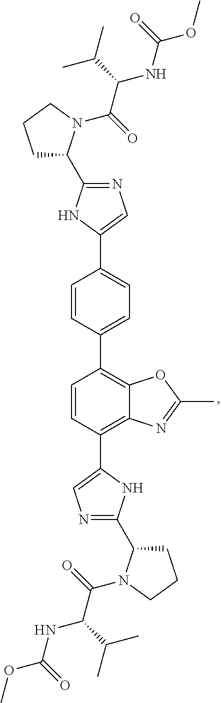

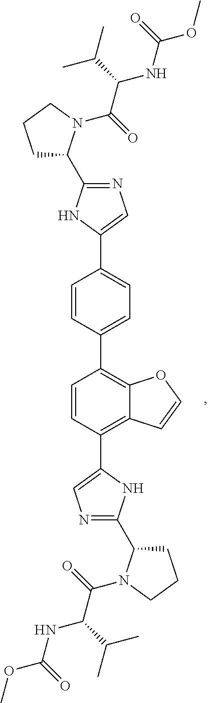

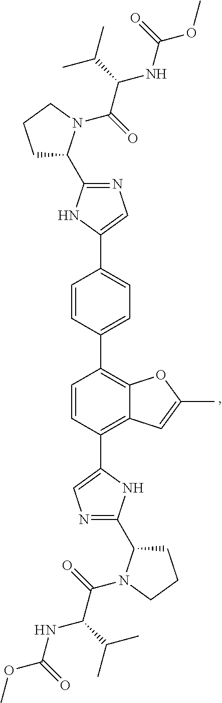

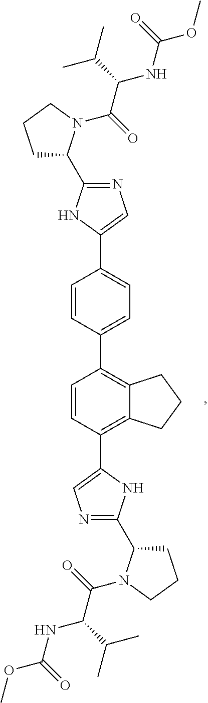

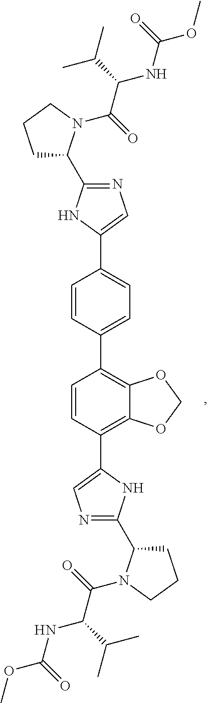

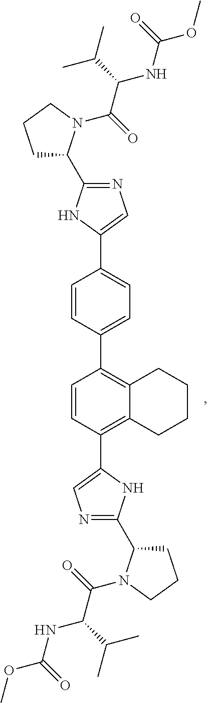

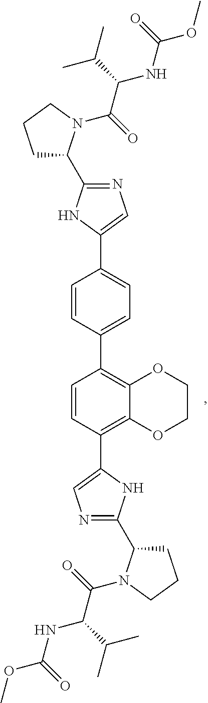

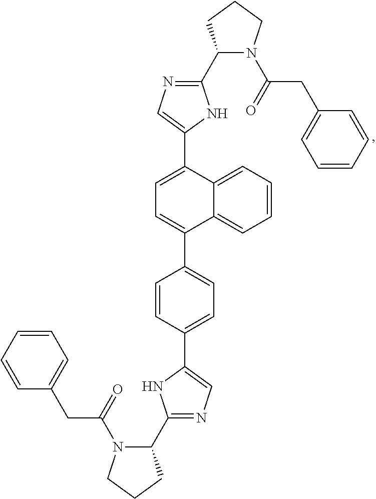

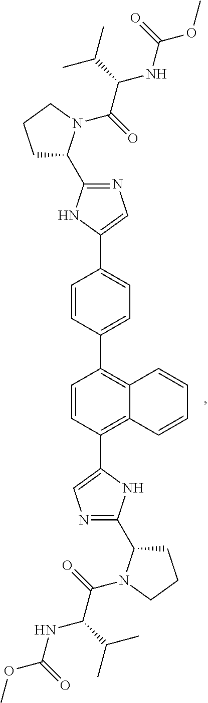

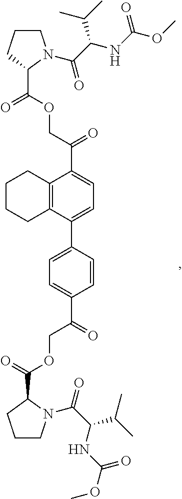

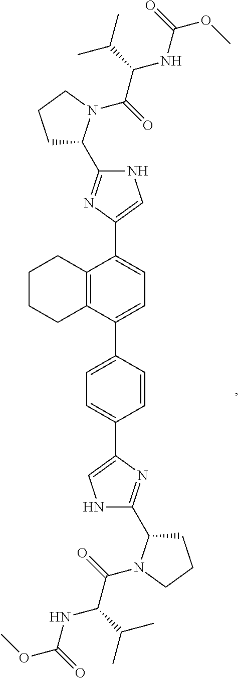

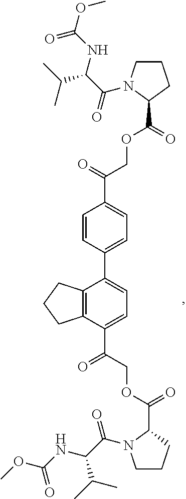

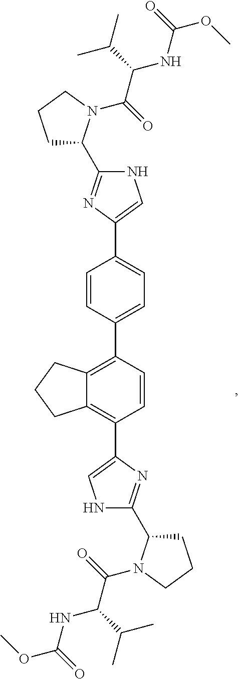

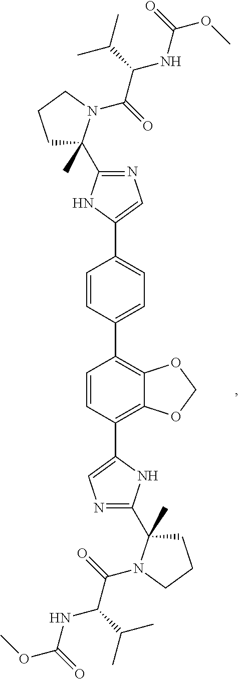

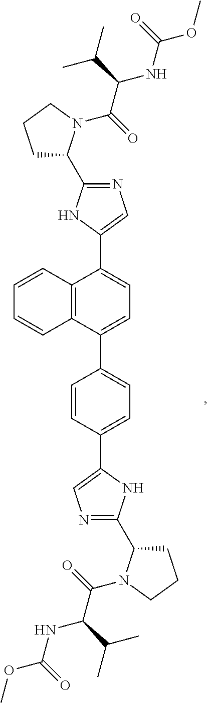

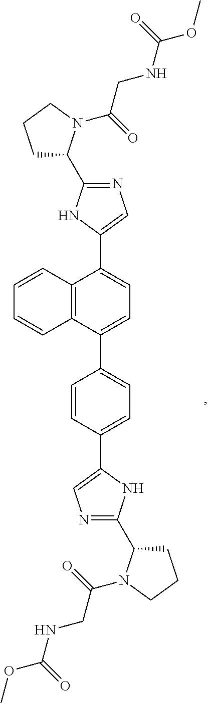

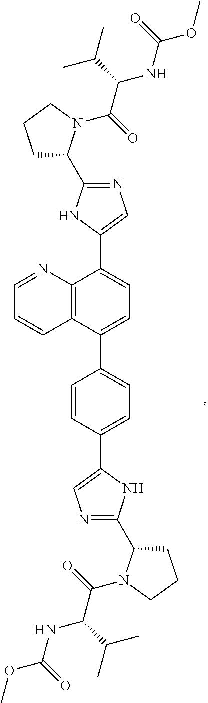

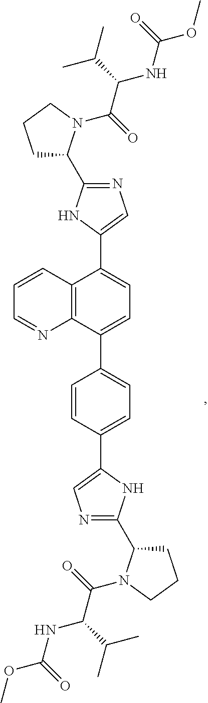

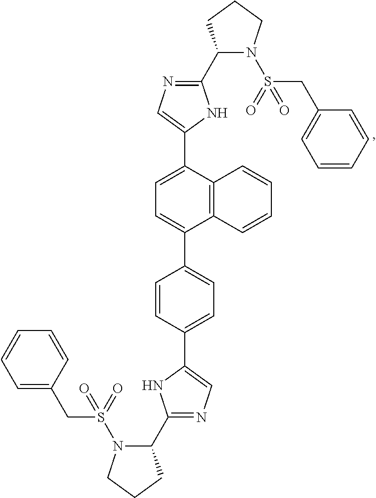

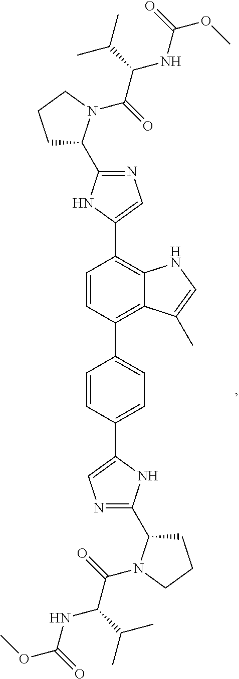

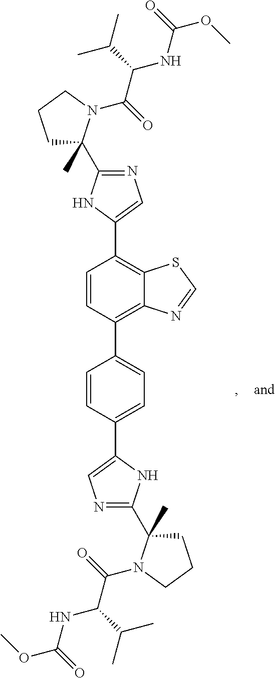

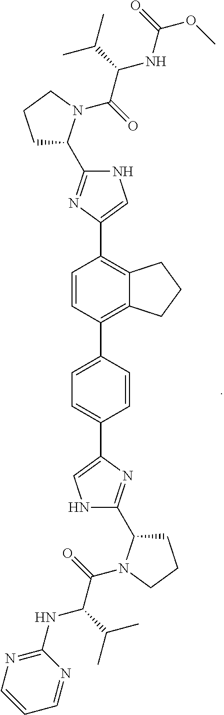

### Preparation of Compounds

### Section I

### Example I-I

### Preparation of Compound 301 and 302

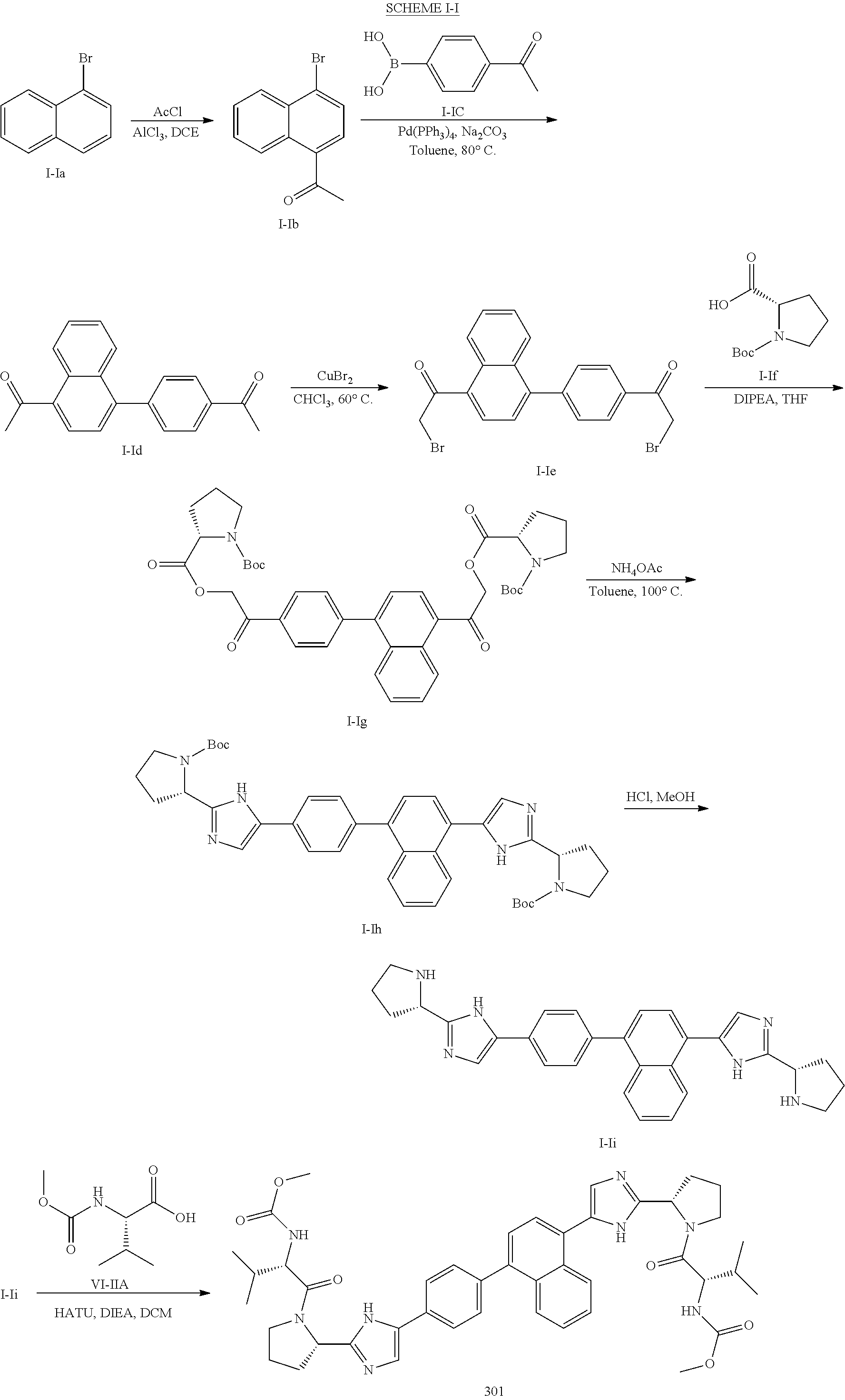

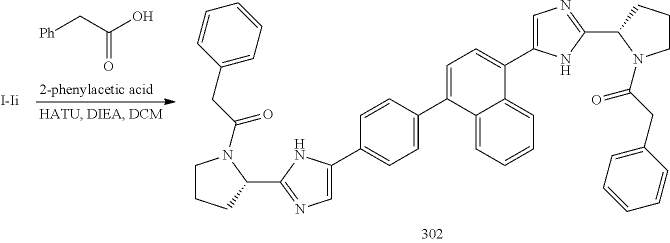

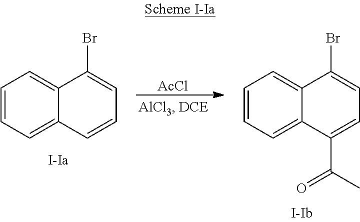

### General Procedure I-A

A solution of 1-Bromo-naphthalene (I-Ia; 2 g, 9.6 mmol) and acetyl chloride (0.84 mL, 11.6 mmol) in 1,2-dichloroethane (30 mL) was cooled to 0° C. and aluminum chloride (2.88 g, 21.6 mmol) was added portion wise. The mixture was stirred at r.t. for 24 hours. The reaction mixture was poured into ice-water (100 mL). The two layers were separated and the aqueous layer was extracted with EtOAc (150 mL×3). The combined organic layers were dried over magnesium sulfate, filtered and the solvent was removed under reduced pressure to give compound I-Ib as an orange oil (2.16 g, yield 91%). ¹H NMR (400 MHz, CDCl₃) δ 8.6 (m, 1H), 8.3 (m, 1H), 7.8 (d, J=8.0 Hz, 1H), 7.66 (d, J=7.6 Hz, 1H), 7.58 (m, 2H), 2.63 (s, 3H). MS (ESI) m/z (M+H)⁺ 250.

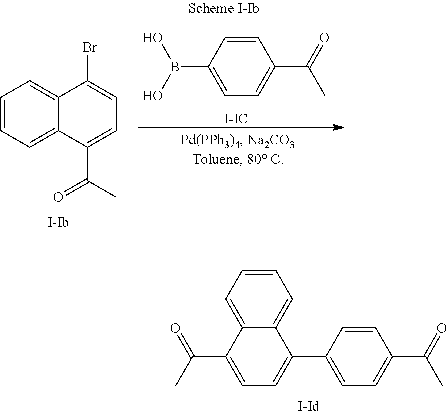

### General Procedure I-B

To a solution of compound I-Ib (2 g, 8.1 mmol) in toluene (20 mL), Na₂CO₃ (0.86 g, 8.1 mmol) and 4-acetylphenylboronic acid (I-IC; 1.6 g, 9.7 mmol) were added, the resulting mixture was purged with nitrogen, then Pd(PPh₃)₄ (848 mg, 0.81 mmol) was added. The reaction mixture was stirred at 80° C. overnight under nitrogen protection. TLC monitored the reaction. After completion of the reaction, the mixture was poured into water, extract with EtOAc (100 mL×3), the combined organic layers were dried over Na₂SO₄, concentrated in vacuo. The residue was purified by chromatography (PE:EA=6:1) to to afford compound I-Id (2 g, yield 86%).

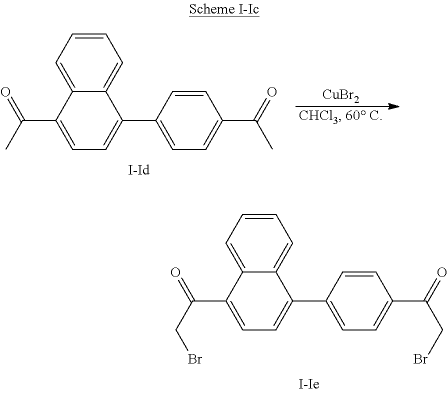

### General Procedure I-C

A suspension of compound I-Id (2 g, 6.9 mmol) in CHCl₃ (20 mL) was treated with CuBr₂ (4.55 g, 20.7 mmol) at 60° C. The mixture was stirred overnight and the precipitate that formed was collected by filtration, washed with EtOAc, and the filtrate was concentrated under reduced pressure to give compound I-Ie, which was used directly in the next step.

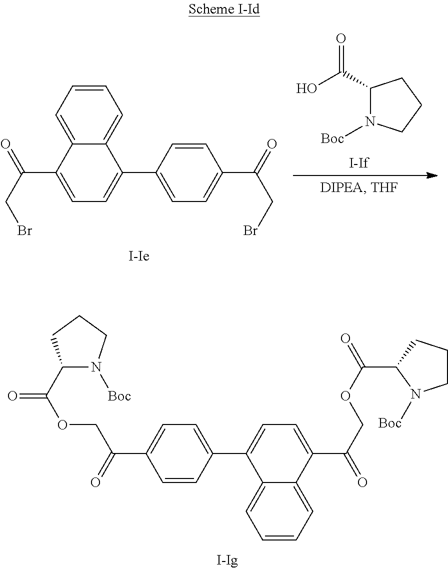

### General Procedure I-D

Diisopropylethylamine (1.78 g, 13.8 mmol) and N-Boc-proline (I-If; 2.97 g, 13.8 mmol) were added to a suspension of compound I-Ie (6.9 mmol) in tetrahydrofuran (18 mL). The resulting mixture was stirred for 1 h as the solids dissolved. The reaction mixture was quenched by the addition of 13% aqueous sodium chloride (20 mL). The layers were separated, and the organic layer was mixed with toluene (50 mL) and concentrated to a volume of 40 mL. The solution, which contained compound I-Ig, was used in the next step.

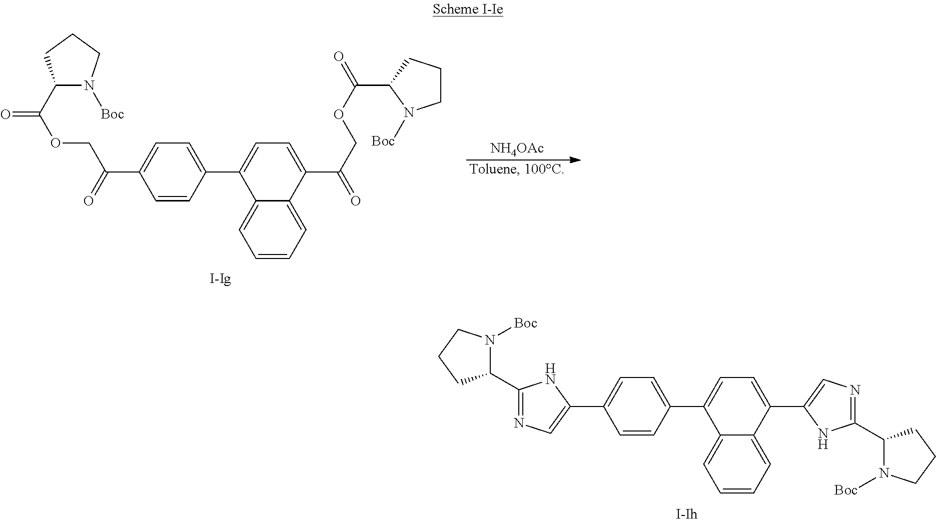

### General Procedure I-E

The solution of compound I-Ig, obtained in the previous experiment, was treated with ammonium acetate (13.9 g, 181 mmol) and heated to 95-100° C. overnight. Concentrated, and the residue obtained was purified by column chromatography (PE:EA=1:1) to afford compound I-Ih (600 mg, 13% over three steps). MS (ESI) m/z (M+H)⁺ 675.

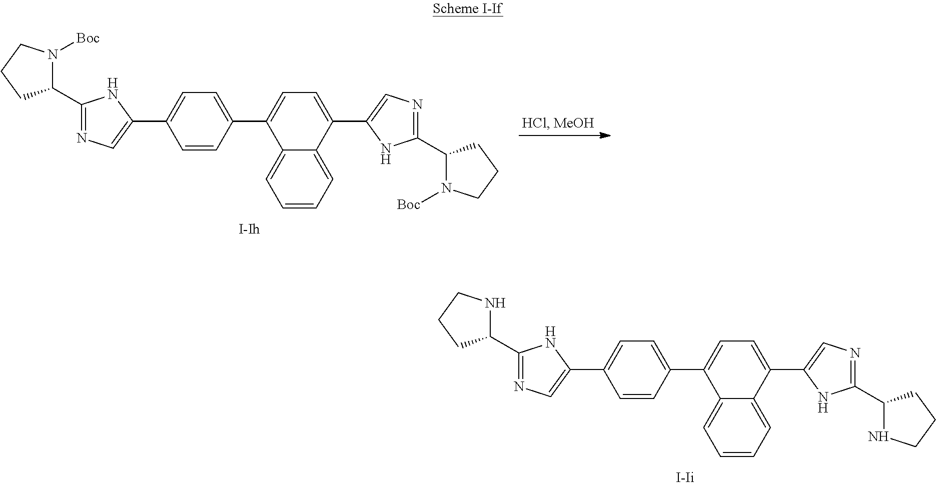

### General Procedure I-F

Aqueous hydrochloric acid (6 M, 6.5 mL) was added to a suspension of compound I-Ih (600 mg, 0.89 mmol) in methanol (10 mL). The resulting mixture was heated to 50° C. with stirring overnight and concentrated to dryness to yield compound I-Ii as a yellow-green solid as HCl salt (380 mg, yield 90%). MS (ESI) m/z (M+H)⁺ 475.3.

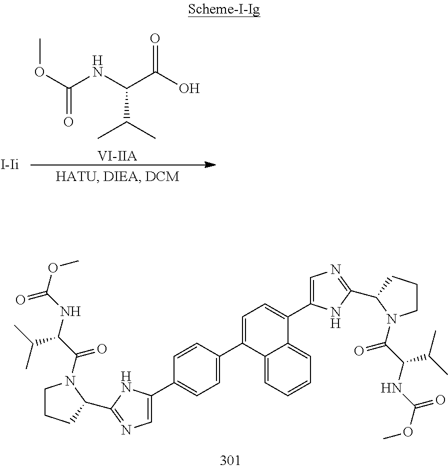

### General Procedure I-G

To solution of compound I-Ii (50 mg, 0.105 mmol) in anhydrous DCM (5 mL), compound VI-IIA (36.7 mg, 0.21 mmol) and DIPEA (32.2 mg, 0.25 mmol) were added, then HATU (79.8 mg, 0.21 mmol) was added under the protection of N₂. The resulting mixture was stirred at r.t. overnight. TLC monitored the reaction. After completion of the reaction, the reaction mixture was poured into water (10 mL), extracted with CH₂Cl₂ (30 mL×3), the combined organic layers were dried over Na₂SO₄, concentrated in vacuo. The residue was purified by Prep-HPLC to give compound 301 as a white solid (21 mg, yield 24%). MS (ESI) m/z (M+H)⁺ 789.4.

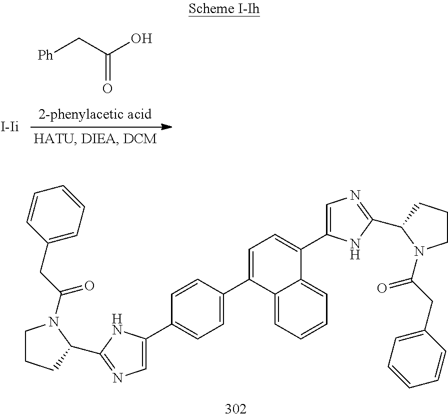

### General Procedure I-H

The procedure for the preparation of compound 302 is similar to that of preparation of compound 301 as described in General Procedure I-G. 120 mg, yield 40%, white solid. MS (ESI) m/z (M+H)⁺ 697.5. 13 mg, yield 19%. white solid. MS (ESI) m/z (M+H)⁺ 711.2.

### Example I-II

### Preparation of Compound 303 and 304

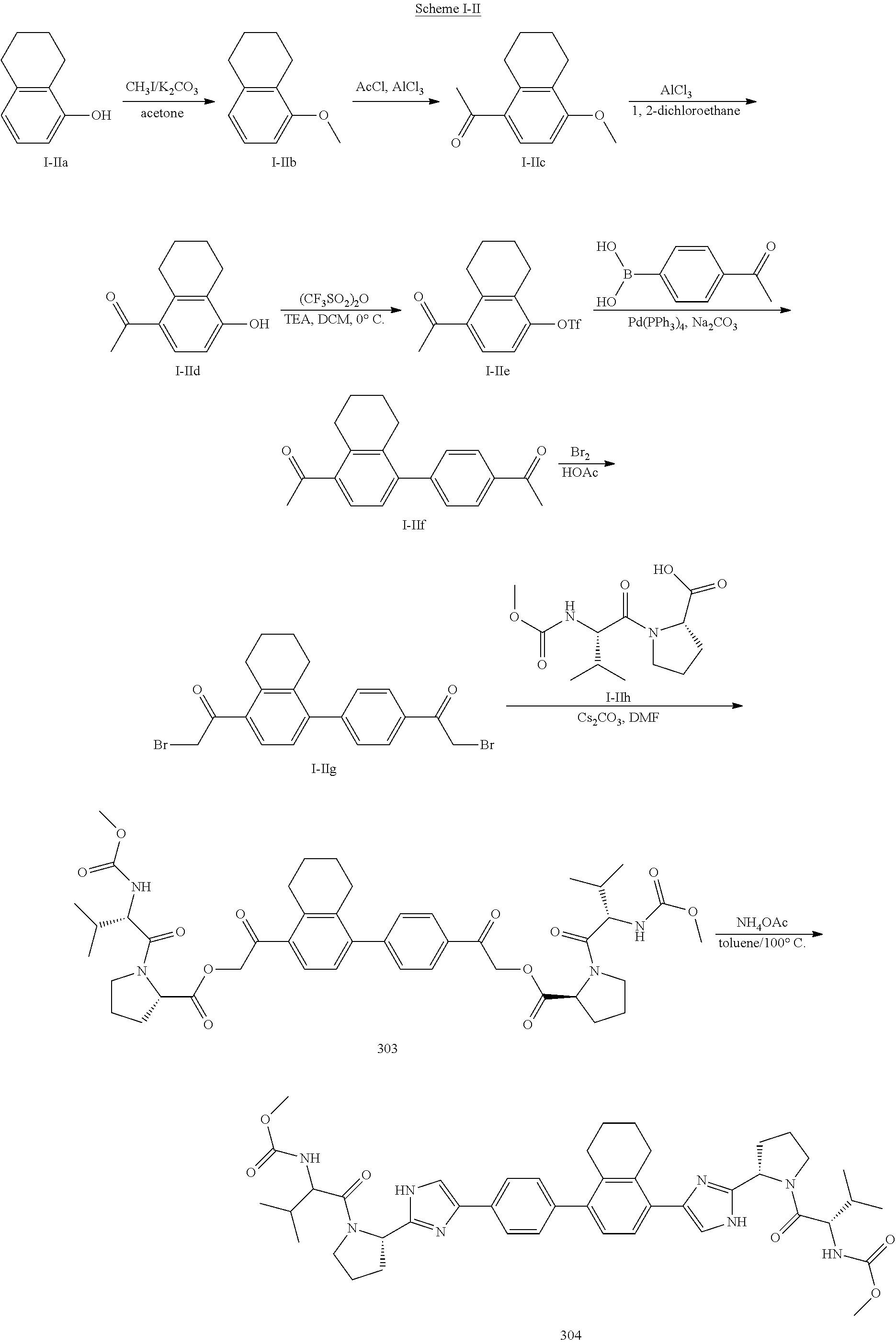

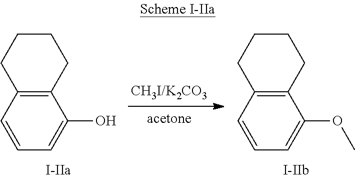

### General Procedure I-I

The mixture of 5,6,7,8-tetrahydronaphthalen-1-ol (IIa; 5 g, 33.74 mmol), CH₃I (4.8 g, 33.74 mmol), and K₂CO₃ (35 mmol) in dry acetone (20 mL) was stirred at reflux overnight. After being cooled to room temperature, the solvent was removed under reduced pressure, and the residue was extracted with ethyl acetate (20 mL×3), washed with water (50 mL) and brine (50 mL). The combined organic layer was dried over anhydrous Na₂SO₄, and concentrated under reduced pressure to afford crude product, which was purified by column chromatography to afford 1,2,3,4-tetrahydro-5-methoxynaphthalene (IIb; 5.47 g, yield: 100%). MS (ESI) m/z (M+H)⁺ 1.63.

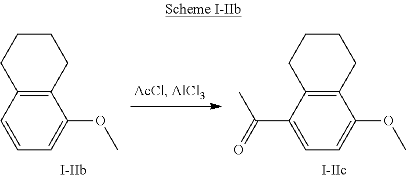

### General Procedure I-J

Acetyl chloride (2.54 g, 32.6 mmol, in 30 mL of 1,2-dichloroethane) was added dropwise to a solution of 1,2,3,4-tetrahydro-5-methoxynaphthalene (IIb; 4.8 g, 29.6 mmol) and anhydrous AlCl₃ (5.08 g, 38.5 mmol) in 100 mL of 1,2-dichloroethane. The reaction mixture was stirred at 0° C. for 30 min. Then the mixture was poured into ice/water (200 mL). The organic layer was separated, washed with brine (20 mL), dried over anhydrous Na₂SO₄, and concentrated under reduced pressure. The residue was purified by column chromatography to afford of compound I-IIc (4.08 g, yield: 80%). ¹H NMR (400 MHz, CDCl₃) δ 7.20 (d, J=8.8 Hz, 1H), 6.83 (d, J=8.8 Hz, 1H), 3.88 (s, 3H), 2.96 (t, 2H), 2.62 (t, 2H), 2.48 (s, 3H), 1.67 (m, 4H); MS (ESI) m/z (M+H)⁺: 205.

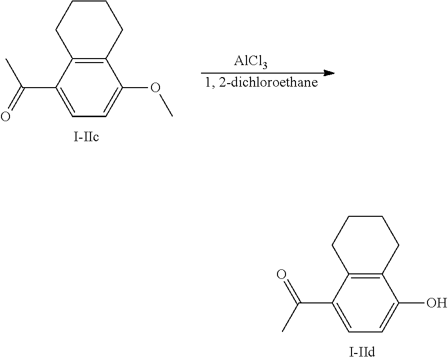

### General Procedure I-K

AlCl₃ (3.9 g, 30 mmol) was added to a solution of compound I-IIc (4 g, 19.6 mmol) in 1,2-dichloroethane (50 mL), the reaction mixture was stirred at reflux for 3 hours. After being cooled to room temperature, the mixture was poured into 100 mL of ice/water. The organic layer was separated, washed with brine (20 mL), dried over sodium sulfate and concentrated under reduced pressure. The residue was purified by column chromatography to afford of compound I-IId (3 g, 80.6% yield). ¹H-NMR (400 MHz, CDCl₃) δ 7.46 (d, J=8.4 Hz, 1H), 6.59 (d, J=8.4 Hz, 1H), 2.96 (m, 2H), 2.58 (m, 2H), 2.48 (s, 3H), 1.76 (m, 2H), 1.67 (m, 2H).

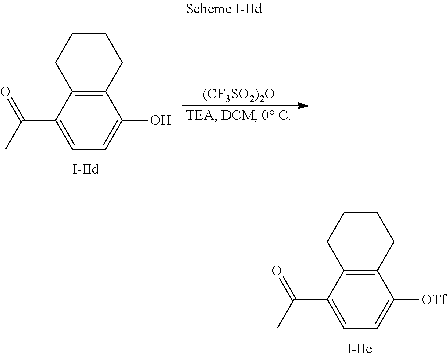

### General Procedure I-L

To a solution of compound I-IId (2.2 g, 11.58 mmol) in dry DCM (50 mL) was added triethylamine (2.34 g, 23.6 mmol) at 0° C. Then trifluoro-methanesulfonic acid anhydride (4.57 g, 16.21 mmol) was added dropwise. The resulting mixture was stirred at 0° C. for 3 hours. Analysis by thin layer chromatography (TLC; petroleum ether:EtOAc=5:1) showed the starting material was consumed completely. The reaction mixture was diluted with DCM (100 mL) and washed with water (50 mL×3). The organic layer was separated, dried over Na₂SO₄, and concentrated under reduced pressure to give compound I-IIe (2.5 g, yield: 97%) as an orange oil, which was used directly in the next step without further purification.

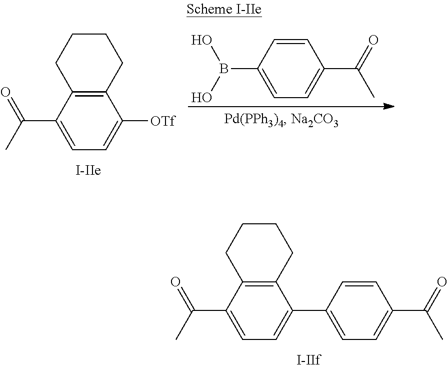

### General Procedure I-M

To a solution of compound I-IIe (2.5 g, 11.5 mmol) in toluene/water (50 mL/5 mL), Na₂CO₃ (2.41 g, 22.7 mmol) and 4-acetylphenylboronic acid (2.85 g, 17.36 mmol) were added, the resulting mixture was purged with nitrogen, then Pd(PPh₃)₄ (0.1 g, catalyzed amount) was added. The reaction mixture was stirred at 80° C. overnight under nitrogen protection. After being cooled to r.t., the mixture was poured into water (100 mL), extract with EtOAc (100 mL×3), the combined organic layers were dried over Na₂SO₄, concentrated under reduced pressure. The residue was purified by chromatography (eluted with petroleum ether:EtOAc=40:1 to 5:1) to afford compound I-IIf (3 g, yield: 91%) as a white solid. ¹H NMR (400 MHz, CDCl₃) δ 8.01 (d, J=8.0 Hz, 2H), 7.49 (d, J=7.6 Hz, 1H), 7.38 (d, J=8.4 Hz, 2H), 7.08 (d, J=7.6 Hz, 1H), 3.02 (m, 2H), 2.65 (s, 3H), 2.60 (s, 3H), 2.56 (m, 2H), 1.76 (m, 2H), 1.70 (m, 2H).

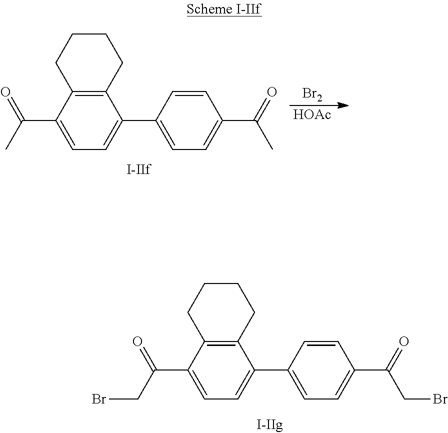

### General Procedure I-N

To a suspension of compound I-IIf (3.2 g, 11 mmol) in HOAc (50 mL) was added a solution of Br₂ (3.51 g, 22 mmol) in HOAc (10 mL) dropwise. The reaction mixture was stirred at 30° C. overnight. Then EtOAc (200 mL) was added and washed with saturated aq.NaHCO₃ (50 mL×3). The organic layer was separated, dried over Na₂SO₄ and concentrated under reduced pressure to give compound I-IIg (3 g, yield: 61%) as an orange oil, which was used directly in the next step

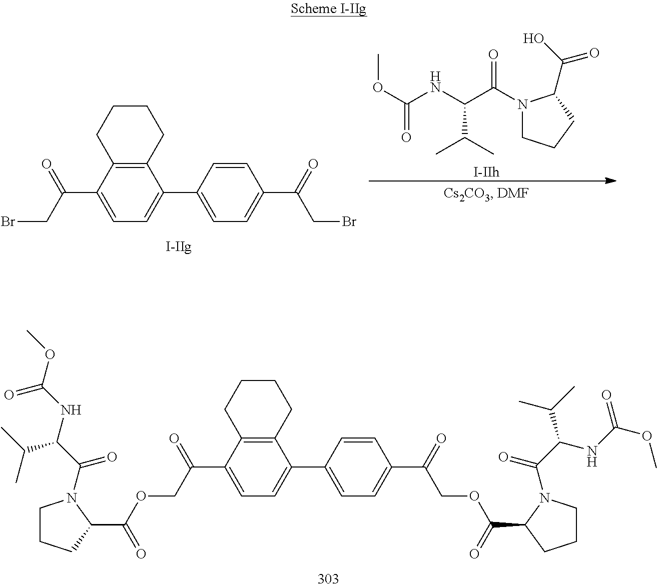

### General Procedure I-O

Compound I-IIh (0.48 g, 1.78 mmol) was added to a suspension of compound I-IIg (0.2 g, 0.44 mmol) and Cs₂CO₃ (0.58 g, 1.78 mmol) in DMF (10 mL). The resulting mixture was stirred at r.t. overnight. Then the reaction mixture was diluted with EtOAc (100 mL) and washed with water (10 mL×5). The organic layer was dried over Na₂SO₄, and concentrated under reduced pressure to afford crude product, which was purified by Prep-HPLC to give compound 303 (0.1 g, yield: 27%) as a white solid. ¹H NMR (300 MHz, CDCl₃) δ 7.94 (d, J=5.4 Hz, 2H), 7.40 (m, 3H), 7.07 (d, J=8.8 Hz, 1H), 5.57 (br, 1H), 5.32 (m, 4H), 5.01 (br, 1H), 4.70 (m, 2H), 4.35 (m, 2H), 3.75 (m, 10H), 2.96 (m, 2H), 2.56 (m, 2H), 2.38 (m, 5H), 2.12 (m, 5H), 1.74 (m, 4H), 1.01 (m, 12H). MS (ESI) m/z (M+H)⁺ 833.3.

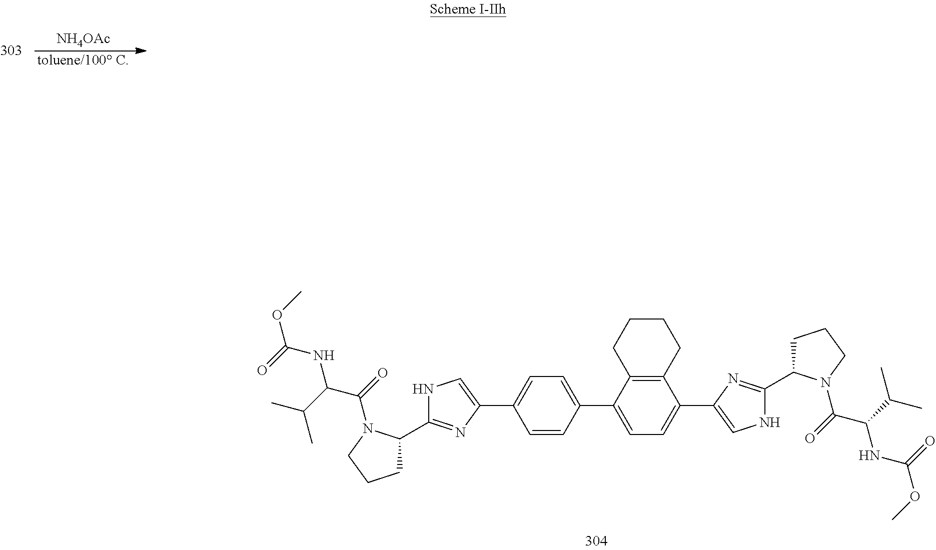

### General Procedure I-P

To a solution of compound 303 (0.1 g, 0.12 mmol) in dry toluene (10 mL) was added ammonium acetate (0.1 g, 1.2 mmol). The resulting mixture was stirred at reflux overnight. After being cooled to room temperature, the mixture was diluted with water (50 mL) and extracted with EtOAc (50 mL×3). The combined organic layers were dried over Na₂SO₄, concentrated under reduced pressure. The residue was purified by Prep-HPLC to afford compound 304 (50 mg, yield: 50%) as a white solid. ¹H NMR (400 MHz, CDCl₃) δ 7.65 (m, 2H), 7.23 (m, 4H), 7.03 (m, 2H), 5.65 (m, 2H), 5.25 (m, 2H), 4.32 (m, 2H), 3.91 (m, 2H), 3.69 (m, 10H), 2.78 (m, 4H), 2.60 (s, 2H), 2.38 (br, 2H), 2.20 (br, 2H), 2.05 (br, 2H), 1.98 (br, 2H), 1.72 (m, 4H), 0.89 (s, 12H). MS: (ESI) m/z (M+H)⁺ 793.3.

### Example I-III

### Preparation of Compound 305, and 306

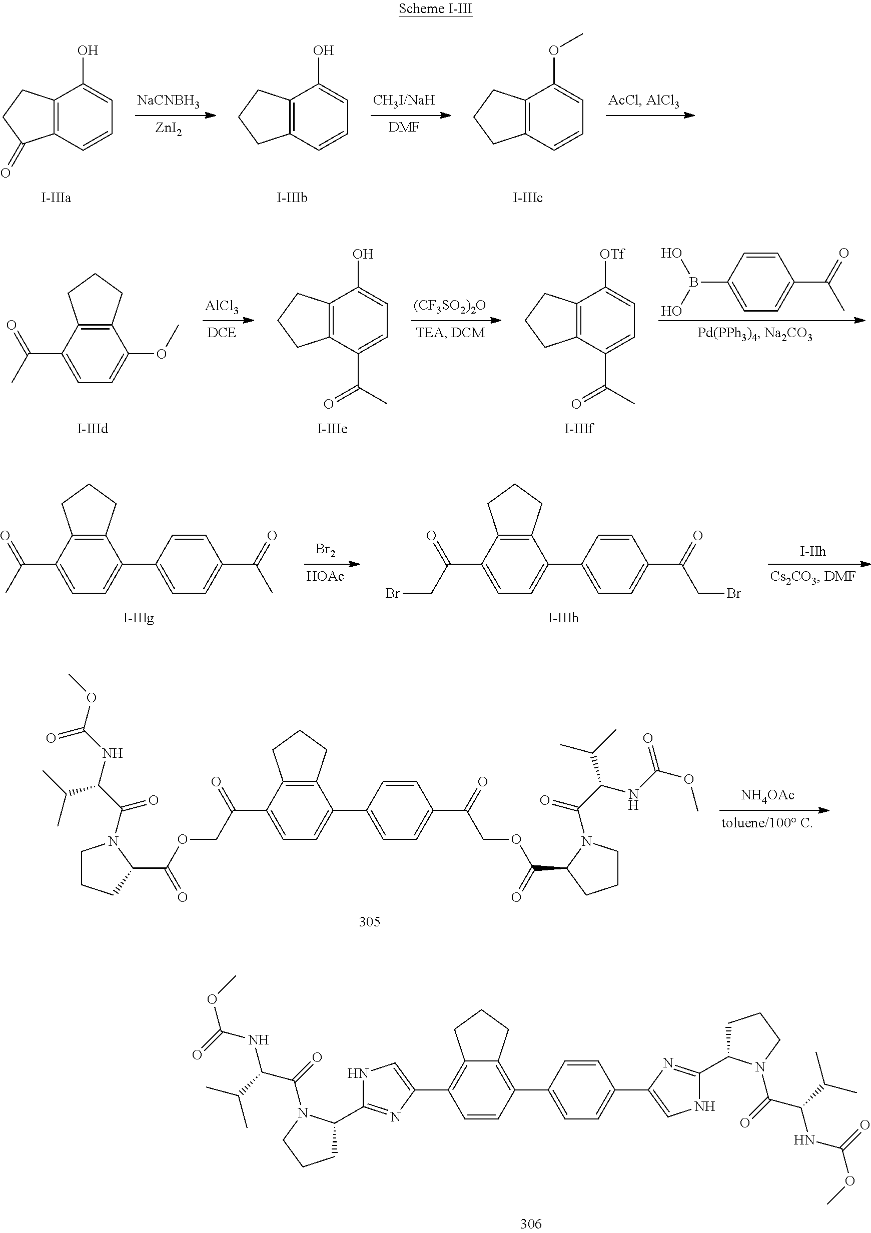

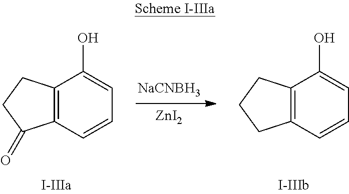

### General Procedure I-Q

NaCNBH₃ (6.4 g, 101.1 mmol) was added to the mixture of compound I-IIIa (5.0 g, 33.7 mmol) and zinc iodide (32.3 g, 101.1 mmol) in dichloroethane (100 mL), the mixture was stirred at reflux for 2 hours. The reaction mixture was then filtered through SiO₂ while still warm, eluding further with dichloroethane. The filtrate was collected and concentrated under reduced pressure. The residue was added to diethyl ether and the resulting white precipitate was filtered off. The filtrate was collected and concentrated in vacuo, then purified by flash column to give compound I-IIIb (3 g, yield: 66%). ¹H NMR (400 MHz, CDCl₃): 7.02 (m, 1H), 6.80 (d, J=5.2 Hz, 1H), 6.61 (m, 1H), 2.91 (m, 4H), 2.05 (m, 2H).

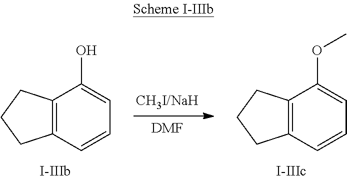

### General Procedure I-R

To a solution of compound I-IIIb (2.9 g, 21.6 mmol) in 30 mL of DMF was added NaH (0.67 g, 28.1 mmol) at 0° C. After addition, CH₃I (3.68 g, 25.9 mmol) was added, and the reaction mixture was stirred at r.t. for 2 hours. Then water (10 mL) was added dropwise, and the mixture was extracted with ethyl acetate (20 mL×3). The organic layer was separated, dried over anhydrous Na₂SO₄, and concentrated under reduced pressure to afford compound I-IIIc (2.5 g, yield: 78%). which was used to the next step directly.

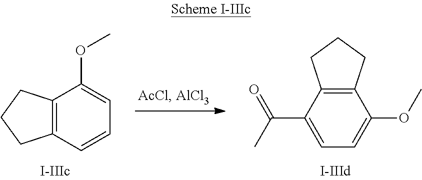

### General Procedure I-S

To a solution of compound I-IIIc (2.5 g, 16.9 mmol) and anhydrous AlCl₃ (2.9 g, 21.8 mmol) in DCM (30 mL) was added dropwise a solution of acetyl chloride (1.6 g, 20.3 mmol) in 10 mL of DCM. After addition, the reaction mixture was stirred at r.t. overnight. Then the solution was poured into ice/water (20 mL). The organic layer was separated, washed with water (20 mL) and brine (20 mL), dried over anhydrous Na₂SO₄, and concentrated under reduced pressure. The residue was purified by column chromatography to afford of compound I-IIId (2.5 g, yield: 78%). ¹H NMR (400 MHz, CDCl₃) δ 7.67 (d, J=8.4 Hz, 1H), 6.64 (d, J=8.4 Hz, 1H), 3.81 (s, 3H), 2.96 (t, J=7.2 Hz, 2H), 2.96 (t, J=7.2 Hz, 2H), 2.48 (m, 2H).

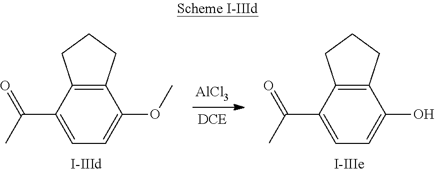

### General Procedure I-T

AlCl₃ (2.1 g, 15.8 mmol) was added to a solution of compound I-IIId (2.5 g, 13.1 mmol) in 1,2-dichloroethane (30 mL), the reaction mixture was stirred at reflux overnight. After being cooled to room temperature, the mixture was poured into 50 mL of ice/water. The organic layer was separated, washed with brine (20 mL), dried over sodium sulfate, and concentrated to give the crude product, which was purified by column chromatography to afford of compound I-IIIe (1.0 g, yield: 43.5%).

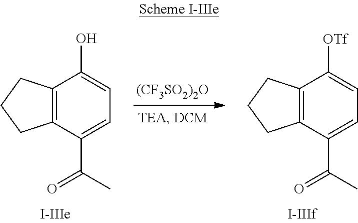

### General Procedure I-U

Tf₂O (1.0 g, 3.6 mmol) was added to a solution of compound I-IIIe (0.5 g, 2.8 mmol) and TEA (0.57 g, 5.6 mmol) in dry DCM (10 mL) at 0° C. The resulting solution was stirred at 0° C. for 2 hours. TLC (petroleum ether:EtOAc=5:1) showed the starting material was consumed completely. The reaction mixture was diluted with DCM (10 mL) and washed with water (5 mL). The organic layer was dried over Na₂SO₄, filtered and concentrated under reduced pressure to give compound I-IIIf (0.65 g, yield: 73.9%). The crude product was used directly in the next step without further purification.

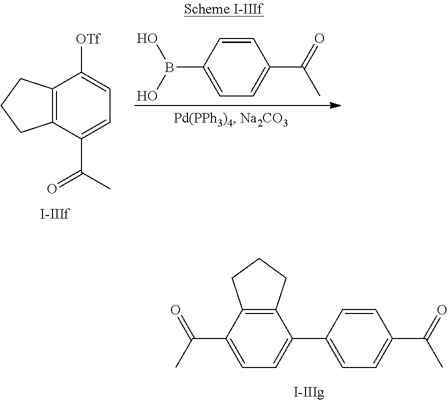

### General Procedure I-V

To a solution of compound I-IIIf (0.05 g, 0.16 mmol) in toluene/H₂O (5 mL/1 mL) was added Na₂CO₃ (0.034 g, 0.32 mmol) and 4-acetylphenylboronic acid (0.047 g, 0.24 mmol). The resulting mixture was purged with nitrogen, then Pd(PPh₃)₄ (5 mg, catalyzed amount) was added. The reaction mixture was stirred at 80° C. overnight under nitrogen protection. After the reaction was completed, the mixture was poured into water (10 mL), extract with EtOAc (20 mL×3), the combined organic layers were dried over Na₂SO₄, concentrated under reduced pressure. The residue was purified by Prep-TLC to afford compound I-IIIg (0.035 g, yield 78%) as a white solid. ¹H NMR (300 MHz, CDCl₃) δ 8.04 (d, J=8.4 Hz, 2H), 7.78 (d, J=7.8 Hz, 1H), 7.54 (d, J=8.1 Hz, 2H), 7.28 (d, J=7.8 Hz, 1H), 3.33 (d, J=7.2 Hz, 2H), 2.95 (d, J=7.2 Hz, 2H), 2.66 (s, 3H), 2.64 (s, 3H), 2.08 (m, 2H).

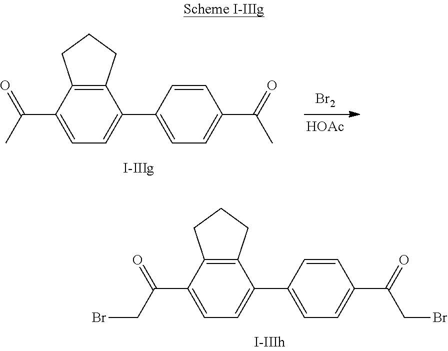

### General Procedure I-W

To a suspension of compound I-IIIg (1.36 g, 4.89 mmol) in HOAc (50 mL) was added a solution of Br₂ (1.56 g, 9.78 mmol, in 5 mL of HOAc) dropwise. The mixture was stirred at 30° C. overnight, then EtOAc (200 mL) was added and the mixture was washed with saturated aq.NaHCO₃ (50 mL×3). The organic layer was separated, dried over Na₂SO₄, and concentrated under reduced pressure to give compound I-IIIh (2 g, yield: 94%) as a yellow solid, which was used directly in the next step.

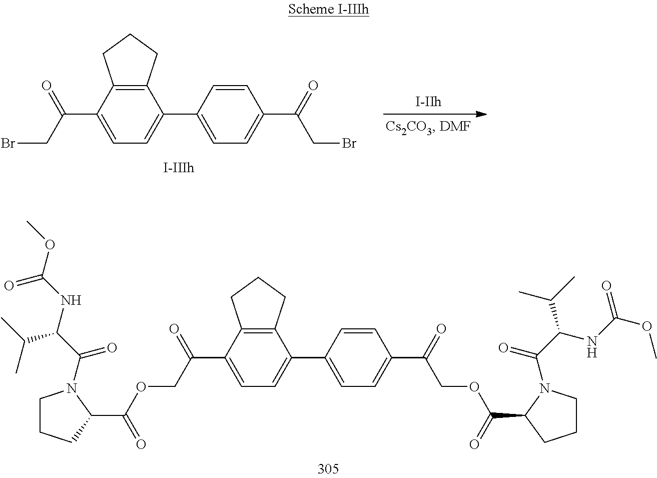

### General Procedure I-X

Compound I-IIh (1.23 g, 4.59 mmol) was added to a suspension of compound I-IIIh (0.5 g, 1.15 mmol) and Cs₂CO₃ (1.5 g, 4.59 mmol) in DMF (20 mL). The resulting mixture was stirred at r.t. overnight. The reaction mixture was diluted with EtOAc (100 mL) and washed with water (10 mL×5). The organic layer was concentrated under reduced pressure to afford the crude product, which was purified by Prep-HPLC to give compound 305 as a white solid (0.6 g, yield: 67%). ¹H NMR (300 MHz, CDCl₃) δ 7.95 (d, J=8.4 Hz, 2H), 7.66 (d, J=5.1 Hz, 1H), 7.53 (d, J=8.4 Hz, 1H), 7.27 (d, J=5.1 Hz, 1H), 5.58 (m, 2H), 5.30 (m, 4H), 4.72 (m, 2H), 4.35 (m, 2H), 3.75 (m, 10H), 3.28 (m, 2H), 2.92 (m, 2H), 2.38 (m, 4H), 2.12 (m, 6H), 1.01 (m, 12H). MS (ESI) m/z (M+H)⁺ 819.4

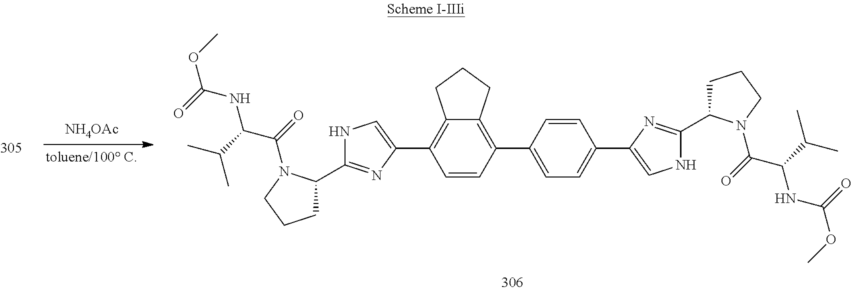

### General Procedure I-Y

To a solution of compound 305 (0.3 g, 0.37 mmol) in dry toluene (15 mL) was added ammonium acetate (0.28 g, 3.7 mmol). The reaction mixture was stirred at reflux overnight. The mixture was diluted with water (50 mL) and extracted with EtOAc (50 mL×3). The combined organic layers were dried over Na₂SO₄, concentrated under reduced pressure. The residue was purified by Prep-HPLC to give compound 306 (0.1 g, yield 35%) as a white solid. ¹H NMR (400 MHz, CDCl₃) δ 7.75 (br, 2H), 7.55 (br, 2H), 7.37 (m, 4H), 7.13 (s, 2H), 7.03 (s, 2H), 5.07 (br, 2H), 4.05 (m, 2H), 3.49 (br, 4H), 3.37 (s, 6H), 3.18 (m, 6H), 2.28-2.14 (m, 10H), 0.93-0.63 (m, 12H). MS (ESI) m/z (M+H)⁺ 779.2

### Example I-IV

### Preparation of Compound 307

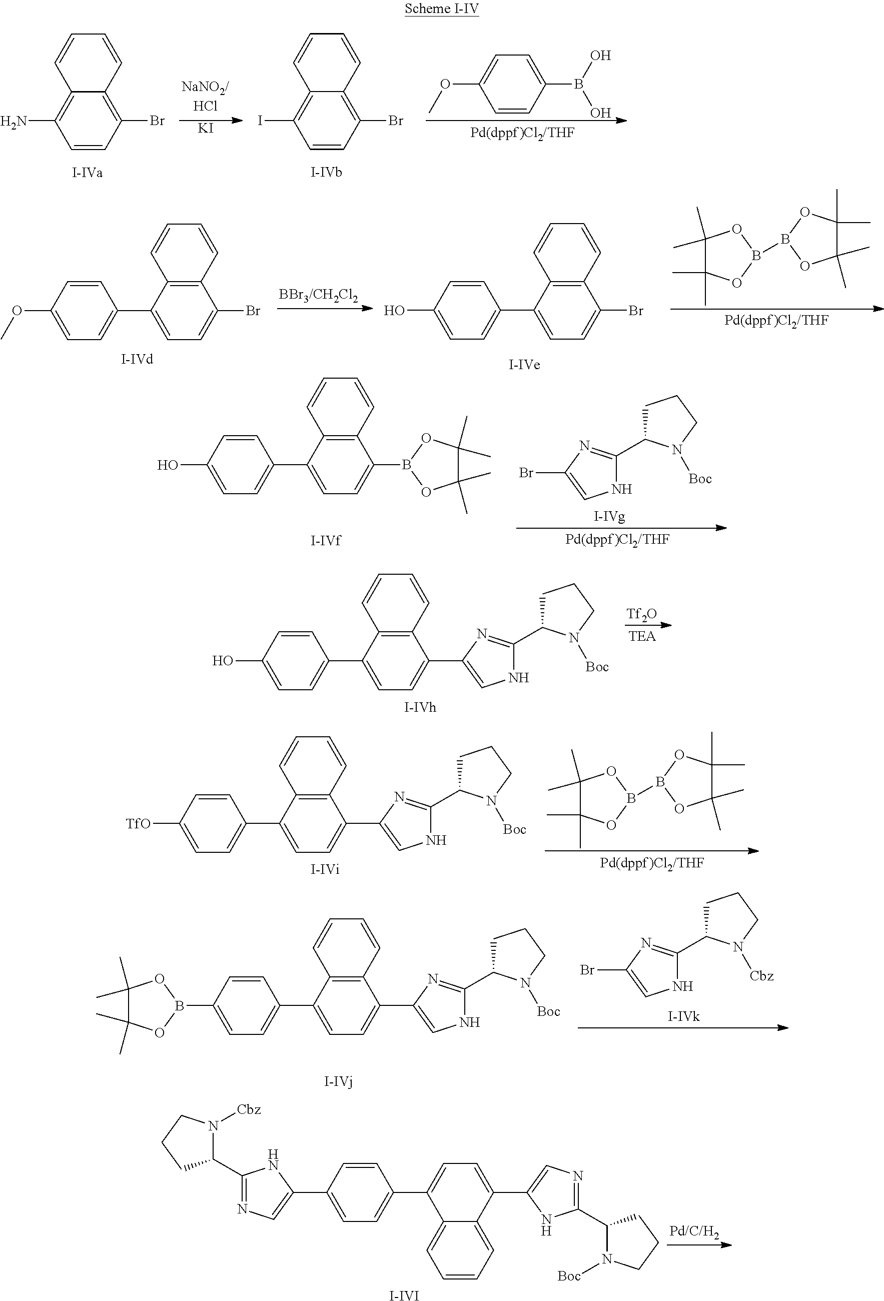

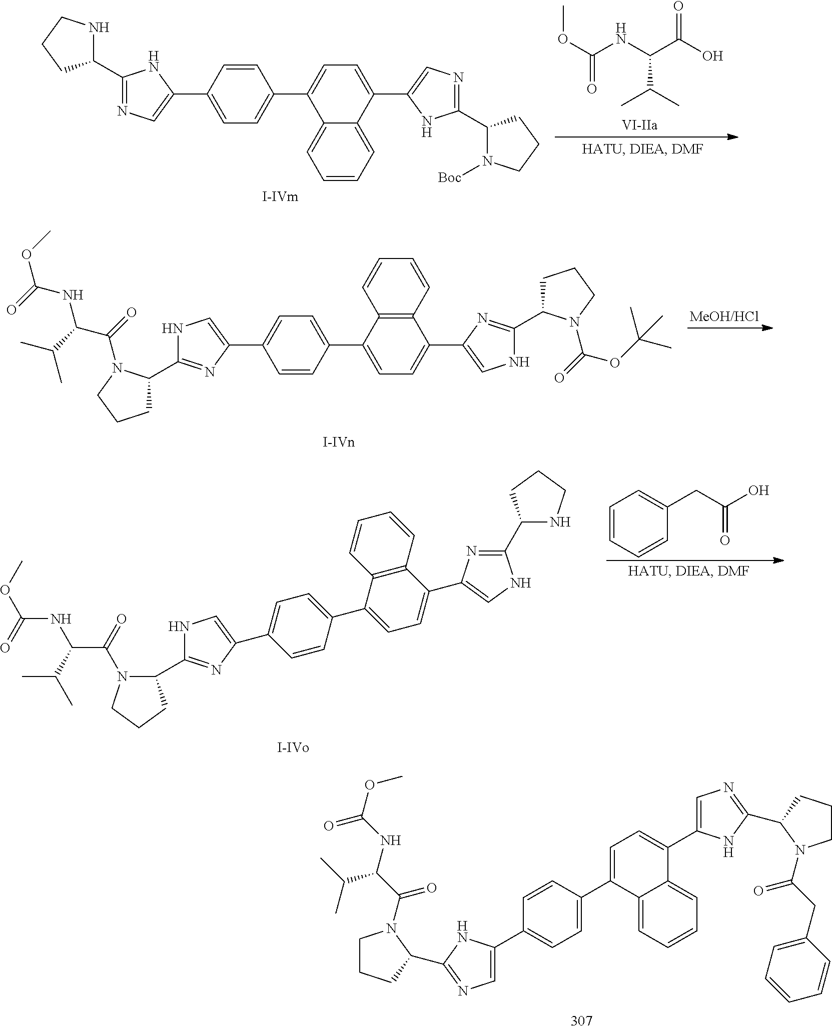

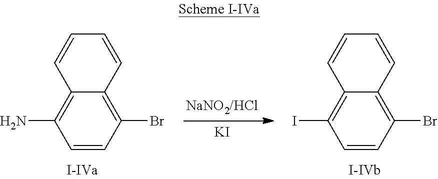

### General Procedure I-Z

To a stirred solution of 4-bromonaphthalen-1-amine (I-IVa) (5.00 g, 22.52 mmol) in 60 mL of concentration HCl was added NaNO₂ (3.10 g, 44.92 mmol) in 10 mL of H₂O at 0° C. under argon. After addition, the solution was stirred for 0.5 hour, then potassium iodide (KI) was added (7.43 g, 44.92 mmol) in 10 mL of H₂O at 0° C. under argon, stirring was continued overnight. The solution was diluted with 100 mL AcOEt, followed by 100 mL H₂O. The aqueous layer was separated and extracted by EtOAc (100 mL×3). The organic layers were combined and washed with the brine, dried over Na₂SO₄, and concentrated in vacuo. The residue was purified by chromatography on silica gel to afford 1-bromo-4-iodonaphthalene (I-IVb) (6 g, yield 83%).

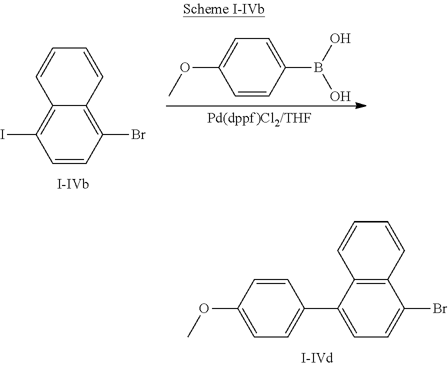

### General Procedure I-AA

The mixture of 1-bromo-4-iodonaphthalene (I-IVb) (6.00 g, 18.01 mmol), 4-methoxyphenylboronic acid (2.74 g, 18.01 mmol), Na₂CO₃ (3.82 g, 36.02 mmol) and Pd(dppf)Cl₂ (658 mg, 0.90 mmol) in 50 mL of THF and 10 mL of H₂O was heated to reflux under argon overnight. The mixture was concentrated, the residue was partitioned between H₂O and DCM, the aqueous phase was extracted with DCM. The combined organic layer was washed with brine, dried over Na₂SO₄, and concentrated. The residue was purified by chromatography on silica gel to afford 1-bromo-4-(4-methoxyphenyl)naphthalene (I-IVd) (4.50 g, yield 63%).

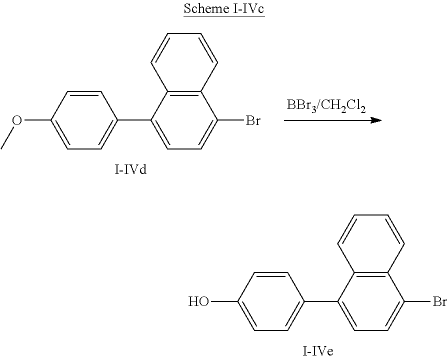

General Procedure I-AB

To a stirred solution of 1-bromo-4-(4-methoxyphenyl)naphthalene (I-IVd) (3 g, 9.58 mmol) in DCM was added dropwise BBr₃ (4.79 g, 19.16 mmol) at −30° C. under argon. After the addition, the solution was stirred for 0.5 hour, and then was warmed slowly to room temperature, stirring for 3 hours. To the solution was added 60 mL of H₂O. The aqueous layer was separated and extracted by EtOAc (60 mL×3). The organic layer was combined and washed with the brine, dried over Na₂SO₄, and concentrated in vacuo. The residue was purified by chromatography on silica gel to afford 4-(1-bromonaphthalen-4-yl)phenol (I-IVd) (2.50 g, yield 78%).

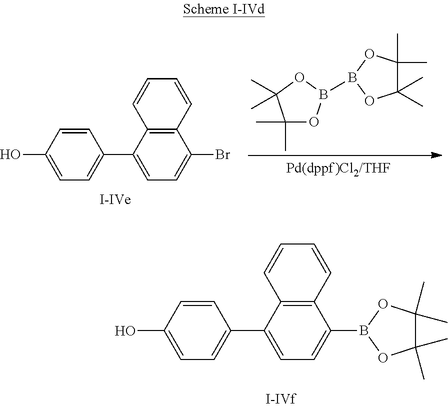

### General Procedure I-AC

A mixture of 4-(1-bromonaphthalen-4-yl)phenol (I-IVd) (2.50 g, 8.36 mmol), Bis(pinacolato)diboron (4.25 g, 16.73 mmol), AcOK (1.63 g, 16.73 mmol) and Pd(dppf)Cl₂ (305 mg, 0.48 mmol) in 40 mL dioxane was heated to reflux under argon for 4 hours. The mixture was concentrated, the residue was partitioned between H₂O and DCM, the aqueous phase was extracted with DCM, and the combined organic layers were washed with brine, dried over Na₂SO₄, and concentrated. The residue was purified by chromatography on silica gel to afford compound I-IVf (2.53 g, yield 89%).

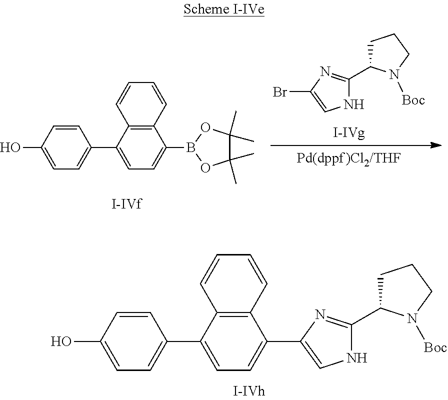

### General Procedure I-AD

A mixture of compound I-IVf (2.53 g, 7.31 mmol), I-IVg (2.31 g, 7.31 mmol), Na₂CO₃ (1.55 g, 15.00 mmol) and Pd(dppf)Cl₂ (270 mg, 0.369 mmol) in 50 mL of THF and 10 mL of H₂O was heated to reflux under argon overnight. The mixture was concentrated, the residue was partitioned between H₂O and DCM, the aqueous phase was extracted with DCM. The combined organic layer was washed with brine, dried over Na₂SO₄, concentrated. The residue was purified by chromatography on silica gel (PE:EA=1:1) to afford compound I-IVh (1.70 g, yield 45%). MS (ESI) m/z (M+H)⁺ 456.4.

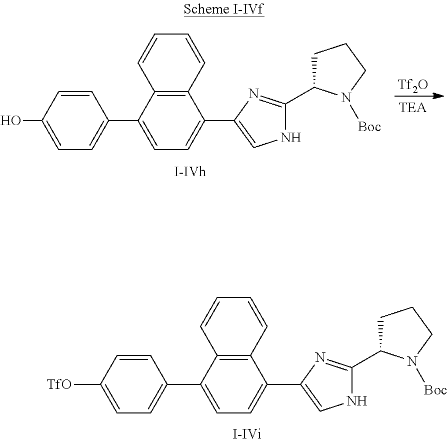

### General Procedure I-AE

To a stirred solution of compound I-IVh (1.70 g, 3.73 mmol) and TEA (0.57 g, 5.64 mmol) in DCM was added dropwise Tf₂O (1.26 g, 4.47 mmol) at −78° C. under argon. After the addition, the solution was stirred for 0.5 hour, and then warmed slowly to the room temperature, stirring for 3 hours. To the solution was added 50 mL H₂O. The aqueous layer was separated and extracted by EA (60 mL×3). The organic layer was combined and washed with the brine, dried over Na₂SO₄, and concentrated in vacuo. The residue was purified by chromatography on silica gel to afford compound I-IVi (1 g, yield 43%).

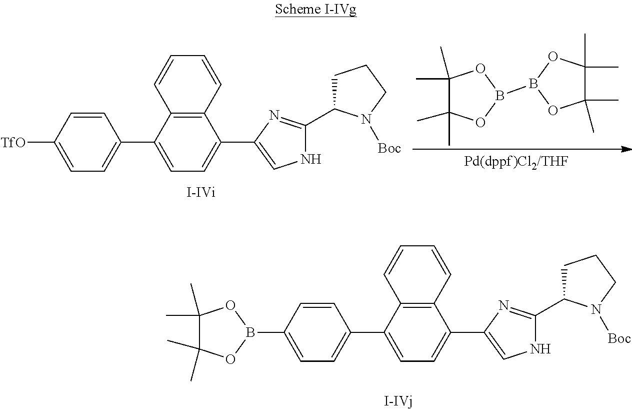

### General Procedure I-AF

A mixture of compound I-IVi (1.00 g, 1.70 mmol), Bis(pinacolato)diboron (0.87 g, 3.40 mmol), AcOK (0.33 g, 3.40 mmol) and Pd(dppf)Cl₂ (62 mg, 0.08 mmol) in 40 mL dioxane was heated to reflux under argon for 4 hours. The mixture was concentrated, the residue was partitioned between H₂O and DCM, the aqueous phase was extracted with DCM, the combined organic layer was washed with brine, dried over Na₂SO₄, concentrated. The residue was purified by chromatography on silica gel to afford compound I-IVj (0.93 g, yield 87%).

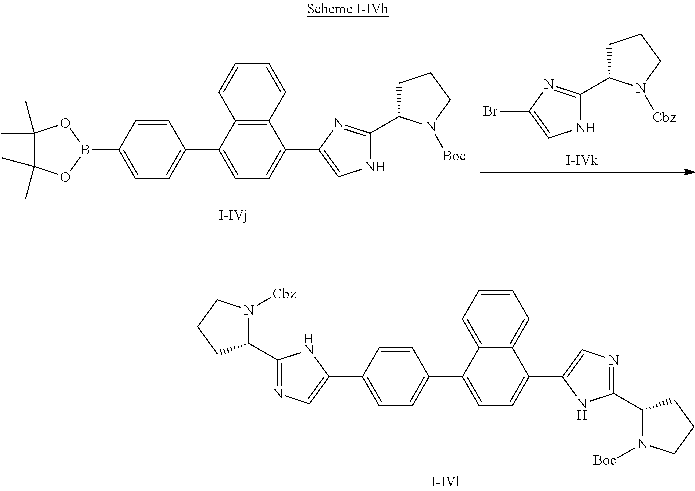

### General Procedure I-AG

A mixture of compound I-IVj (0.93 g, 1.64 mmol), compound I-IVk (0.57 g, 1.64 mmol), Na₂CO₃ (0.35 mg, 3.28 mmol) and Pd(dppf)Cl₂ (60 mg, 0.08 mmol) in 50 mL of THF and 10 mL of H₂O was heated to reflux under argon overnight. The mixture was concentrated, the residue was partitioned between H₂O and DCM, the aqueous phase was extracted with DCM. The combined organic layer was washed with brine, dried over Na₂SO₄, concentrated. The residue was purified by chromatography on silica gel (PE:EA=1:1) to afford compound I-IVl (600 mg, yield 72%). MS (ESI) m/z (M+H)⁺ 707.

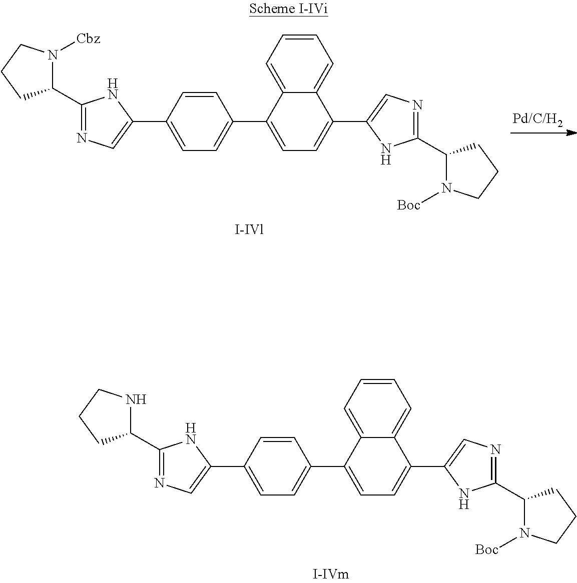

### General Procedure I-AH

Compound I-IVl (600 mg, 0.848 mmol) was dissolved in 20 mL of methanol. After addition of 100 mg of 10 percent Pd-on-charcoal, the mixture was hydrogenated by hydrogen balloon at room temperature for 4 hours, the catalyst was removed by filtration using celite, and the filtrate was concentrated to afford the crude product I-IVm (414 mg, yield 77%). MS (ESI) m/e (M+H)⁺ : 575.3.

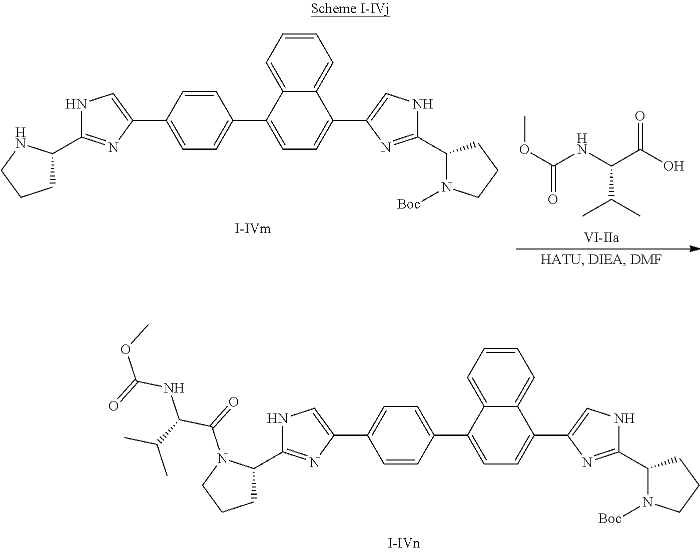

### General Procedure I-AI

To a mixture of compound I-IVm (207 mg, 0.361 mmol), compound VI-IIa (63 mg, 0.361 mmol) and DIPEA (93 mg, 0.361 mmol) in DMF (3 mL) was added HATU (137 mg, 0.361 mmol). The resulting mixture was stirred at room temperature. After completion of the reaction, as observed by disappearance of compound I-IVm by LCMS, the mixture was purified by Prep-HPLC to afford compound I-IVn (72 mg, yield 37%). MS (ESI) m/e (M+H)⁺ : 732.7.

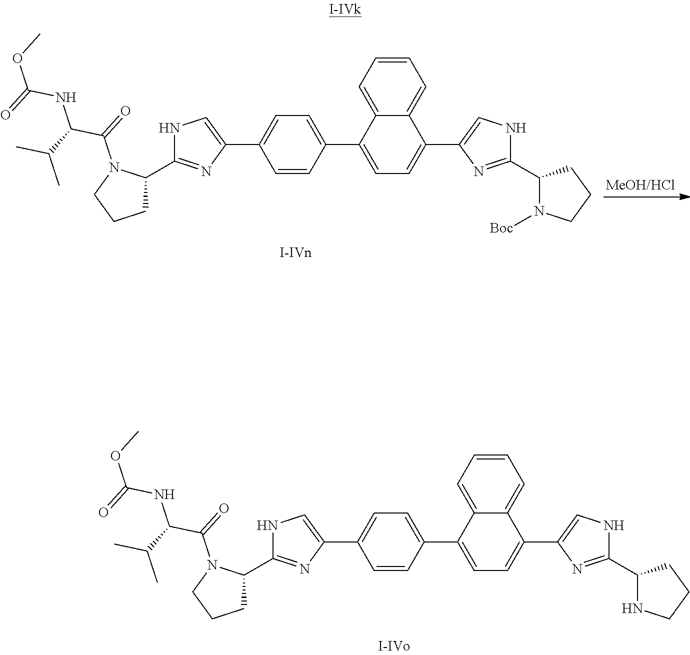

### General Procedure I-AJ

Compound I-IVn (72 mg, 0.11 mmol) was added into HCl/CH₃OH (20 mL, 4M). Then the mixture was stirred at room temperature for 2-3 hrs. After completion of the reaction, the mixture was concentrated under vacuum to afford compound I-IVo (62 mg, yield 92%). MS (ESI) m/e (M+H)⁺ : 632.

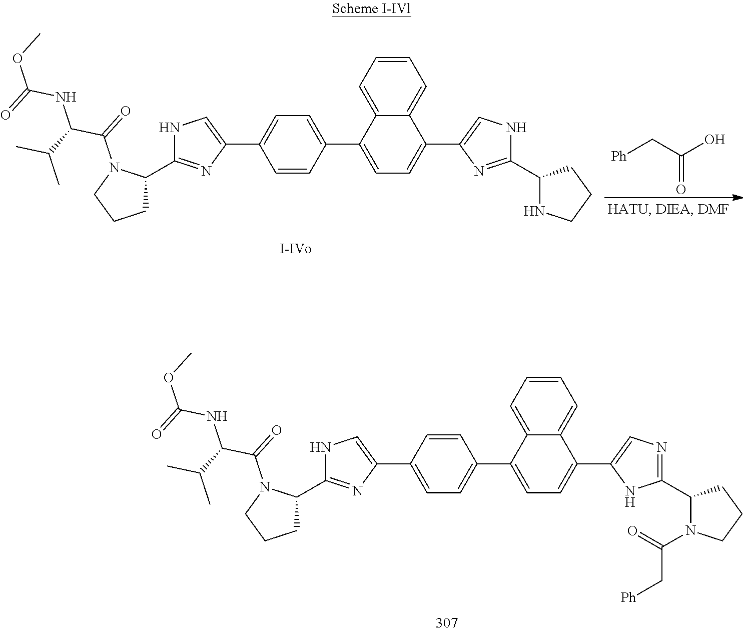

### General Procedure I-AK

To a mixture of compound I-IVo (62 mg, 0.116 mmol), 2-phenylacetic acid (13 mg, 0.116 mmol) and DIPEA (43 mg, 0.116 mmol) in DMF (3 mL) was added HATU (43 mg, 0.116 mmol). The resulting mixture was stirred at room temperature until complete as observed by LCMS. The crude product was purified by Prep-HPLC to afford compound 307 (18 mg, yield 53%). MS (ESI) m/e (M+H)⁺ : 750.6.

### Example I-V

### Preparation of Compound 308

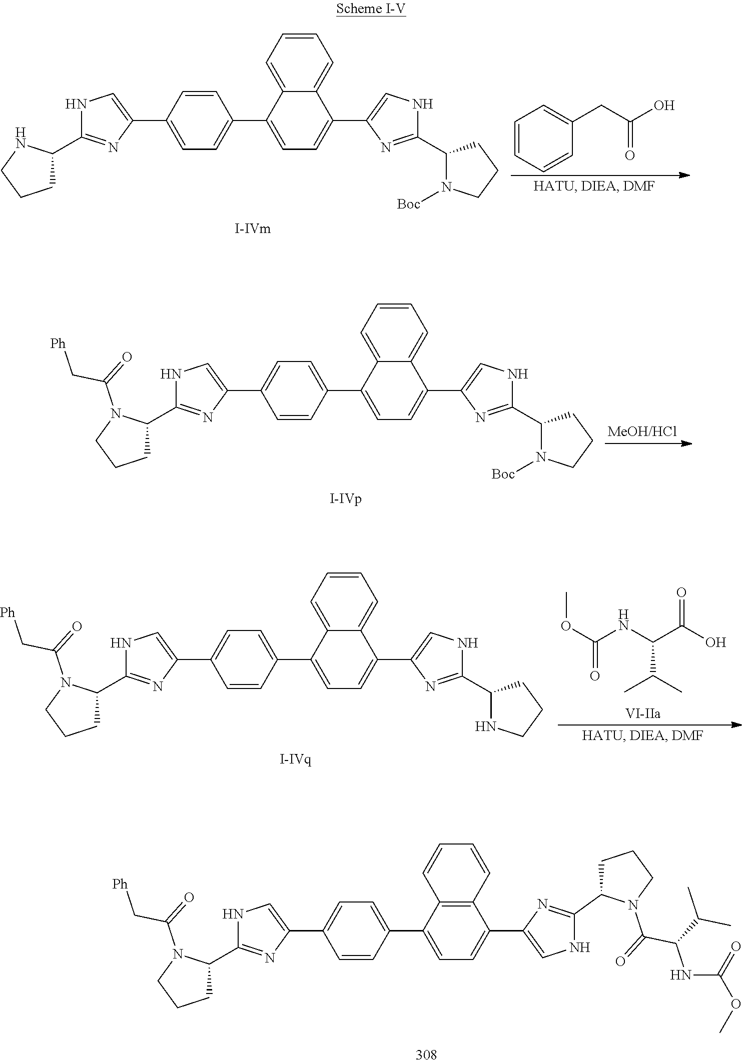

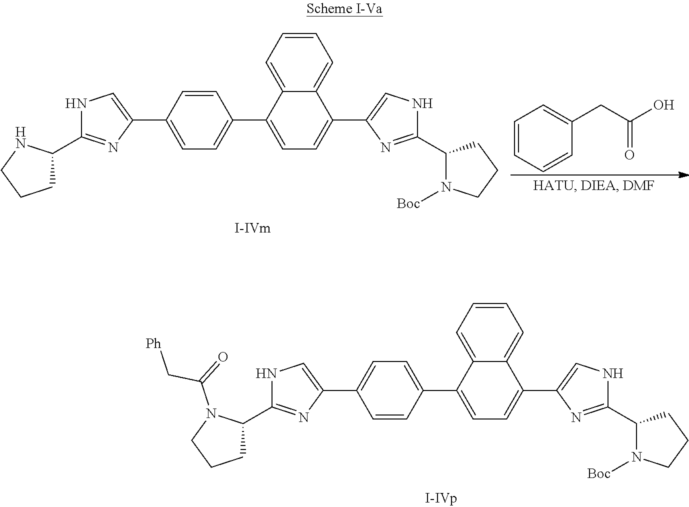

### General Procedure I-AL

To a mixture of compound I-IVm (207 mg, 0.361 mmol), 2-phenylacetic acid (49 mg, 0.361 mmol) and DIPEA (93 mg, 0.361 mmol) in DMF (3 mL) was added HATU (137 mg, 0.361 mmol). The resulting mixture was stirred at room temperature until complete as observed by LCMS. The crude product was purified by Prep-HPLC to afford compound I-IVp (60 mg, yield 28%). MS (ESI) m/e (M+H)⁺: 692.

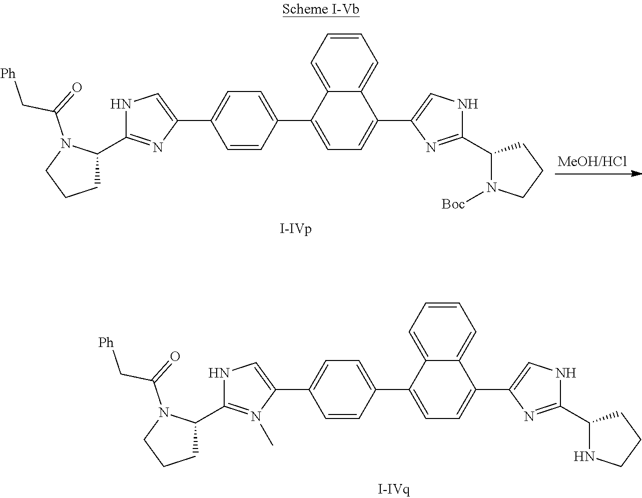

### General Procedure I-AM

Compound I-IVp (60 mg, 0.09 mmol) was added into HCl/CH₃OH (20 mL, 4M). Then the mixture was stirred at room temperature for 2-3 hrs. When the reaction was complete, the mixture was concentrated under vacuum to give compound I-IVq (45 mg, yield 92%). MS (ESI) m/e (M+H)⁺: 592.

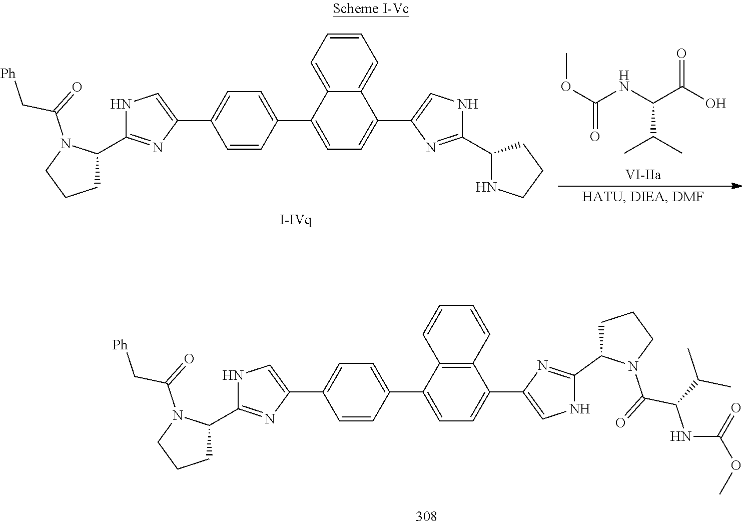

### General Procedure I-AN

To a mixture of compound I-IVq (45 mg, 0.08 mmol), compound VI-IIa (14 mg, 0.08 mmol) and DIPEA (29 mg, 0.08 mmol) in DMF (3 mL) was added HATU (34 mg, 0.08 mmol). The resulting mixture was stirred at room temperature until complete as observed by LCMS. The crude product was purified by Prep-HPLC to afford 308 (20 mg, yield 57%). MS (ESI) m/e (M+H)⁺: 750.6.

### Example I-VI

### Preparation of Compound 309

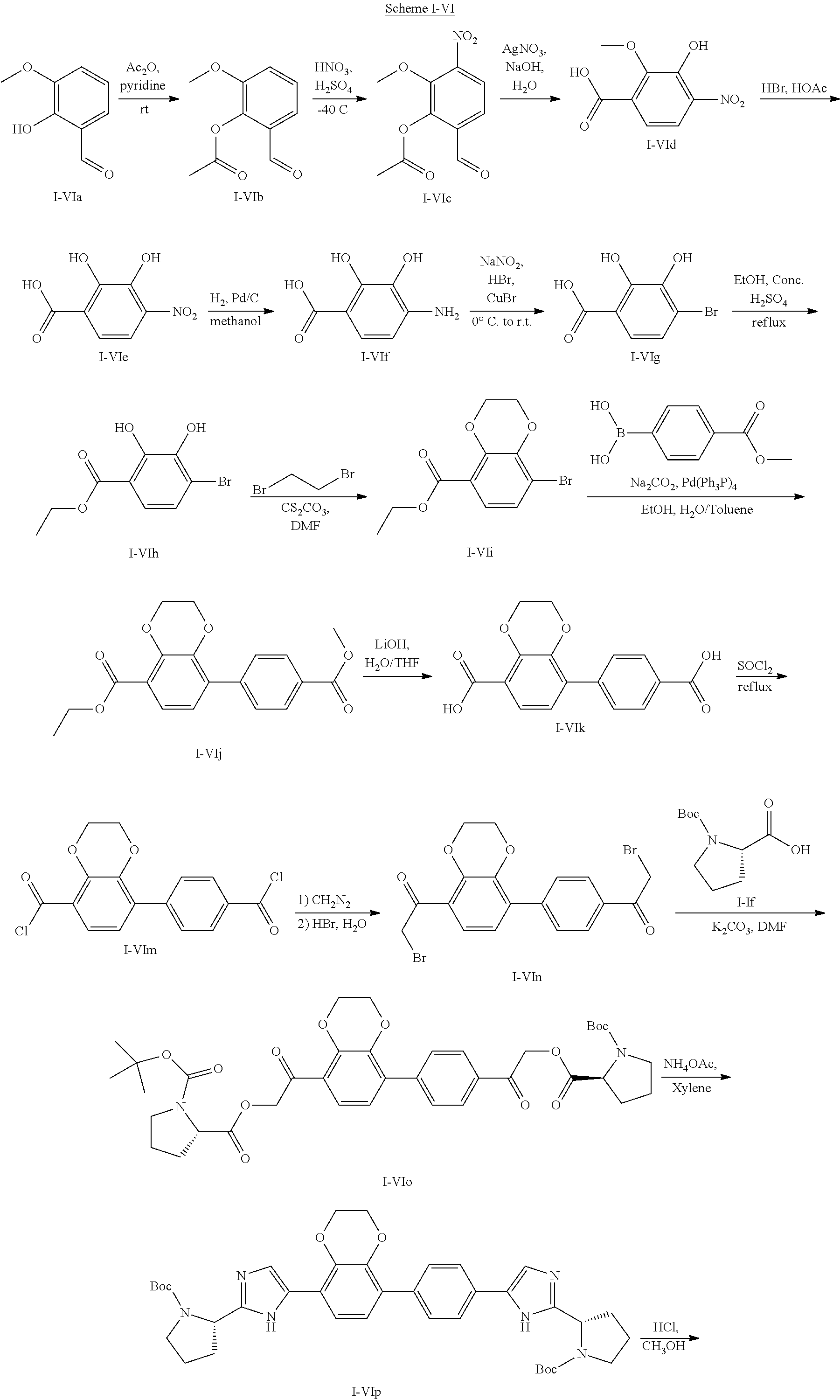

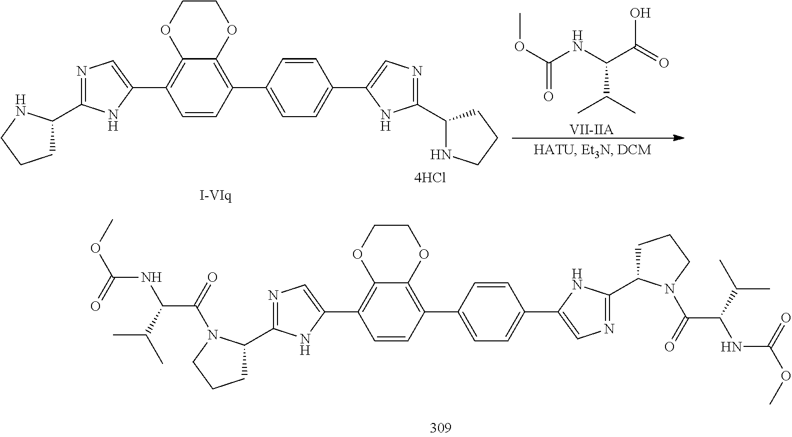

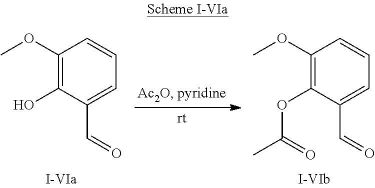

### General Procedure I-AO

To a solution of 2-hydroxy-3-methoxybenzaldehyde (I-VIa) (15.2 g, 100 mmol) in pyridine (50 mL) was added Ac₂O (11.2 g, 110 mmol) and the reaction mixture was stirred at room temperature for 24 hours. The reaction mixture was poured into water and extracted with DCM, washed with aq. HCl (4.0 M) and brine. The organic layer was dried over anhydrous sodium sulfate and the solvent was removed under reduced pressure to provide compound I-VIb (17.9 g, yield 93%) as white solid.

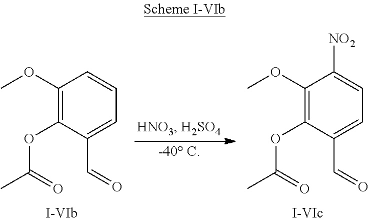

### General Procedure I-AP

A solution of compound I-VIb (9.7 g, 50 mmol) in H₂SO₄ (15 mL) was cooled to −40° C. with a dry-ice bath, fuming HNO₃ (10.0 mL) was slowly added thereto. The reaction mixture was stirred at the same temperature for 5 minutes, then, the reaction mixture was poured into ice-water and extracted with DCM. The organic layer was dried with anhydrous sodium sulfate and removed in vacuo. The residue was purified by column chromatography on silica gel (eluent PE:EtOAc=9:1) to afford compound I-VIc (7.8 g, yield 63%) as yellow solid. ¹H NMR (400 MHz, CDCl3) δ 9.92 (s, 1H), 7.36-7.38 (d, 1H), 7.19-7.21 (d, 1H), 4.01 (s, 3H), 2.10 (s, 3H).

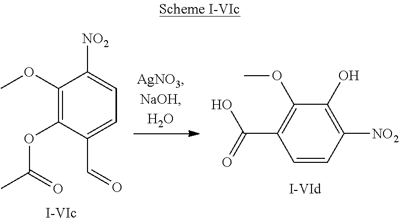

### General Procedure I-AQ

To a mixture of compound I-VIc (10.0 g, 42.0 mmol) in methanol (150 mL) were added NaOH (6.8 g, 170.0 mmol), water (800 mL). The mixture was stirred for 5 minutes, then, AgNO₃ (8.5 g, 50.0 mmol) was added. After addition, the temperature of the reaction mixture was raised to 85° C., then, stirred at the same temperature overnight. The reaction mixture was filtered through a celite and the pH value of the filtrate was adjusted to 2, extracted with EtOAc, and washed with water and brine. The solvent was removed in vacuo to give compound I-VId (5.1 g, yield 56%) as yellow solid.

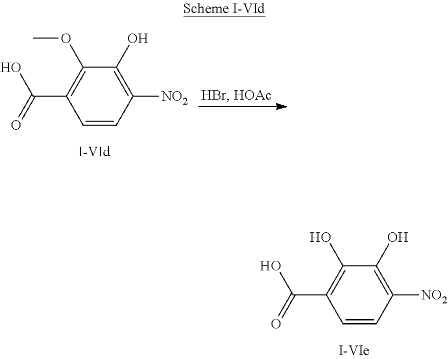

### General Procedure I-AR

To a solution of compound I-VId (5.1 g, 24.0 mmol) in HOAc (60.0 mL) was added 47% aq. HBr (30.0 mL) and the reaction mixture was refluxed for 4 hours. After detection by TLC, the reaction mixture was cooled in an ice-bath and the yellow solid was appeared. The solid was collected by filtration and washed with water and dried to give 2,3-dihydroxy-4-nitrobenzoic acid (I-VIe) (4.0 g, yield 83%) as yellow solid.

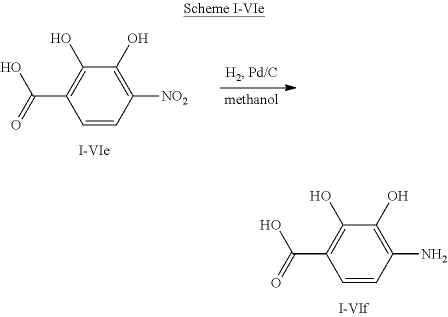

### General Procedure I-AS

To a solution of 2,3-dihydroxy-4-nitrobenzoic acid (I-VIe) (4.0 g, 20.0 mmol) in methanol (100 mL) was added 10% palladium on carbon (0.5 g) and the mixture was hydrogenated with at room temperature at 40 Psi pressure of hydrogen. After no further change was observed on the pressure of hydrogen, the catalyst was filtered through Celite and washed with methanol. The filtration was evaporated to dryness to give 4-amino-2,3-dihydroxybenzoic acid (I-VIf) (4.9 g, yield 98%) as yellow solid.

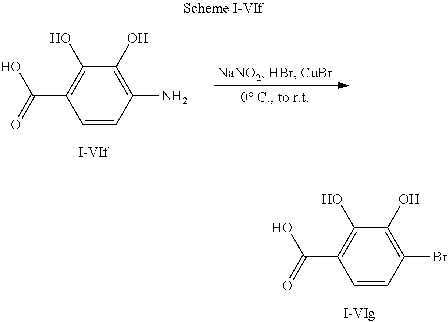

### General Procedure I-AT

4-Amino-2,3-dihydroxybenzoic acid (I-VIf) (4.9 g, 20.0 mmol) was taken up in water (30 ml) containing 48% aq. HBr (8.0 mL) and cooled to 0° C. A solution of NaNO₂ (1.5 g, 22.0 mmol) in water (10.0 mL) was slowly introduced, and the mixture was stirred at 0° C. for 2 hours. A mixture of cuprous bromide (3.1 g, 22 mmol) and hydrobromic acid (8 mL) was added dropwise to the mixture at 0° C. The mixture was stirred at the same temperature for 1 hour, and then stirred at r.t. overnight. The mixture was extracted with ethyl acetate and washed with brine and dried over anhydrous sodium sulfate. The solvent was removed to afford 4-bromo-2,3-dihydroxybenzoic acid (I-VIg) (3.3 g, yield 70%) as yellow solid.

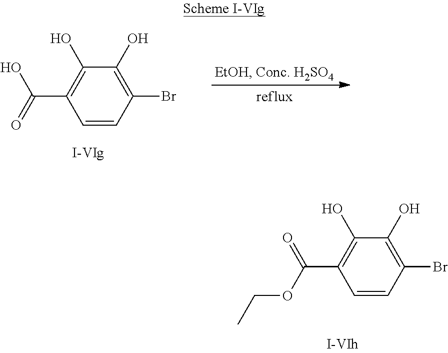

### General Procedure I-AU

To a solution of 4-bromo-2,3-dihydroxybenzoic acid (I-VIg) (3.3 g, 14.0 mmol) in EtOH (100 mL) was added conc.H₂SO₄ (5.0 mL) and the mixture was refluxed for 16 hours. The solvent was removed and the residue was dissolved in ethyl acetate and washed with water, saturated aq. NaHCO₃, and brine. The solvent was removed to give ethyl 4-bromo-2,3-dihydroxybenzoate (I-VIh) (3.5 g, yield 95%) as yellow solid. ¹H NMR (400 MHz, CDCl3) δ 11.14 (s, 1H), 7.20 (d, 1H), 6.96 (d, 1H), 5.93 (br, 1H), 4.34 (q, 2H), 1.34 (t, 3H).

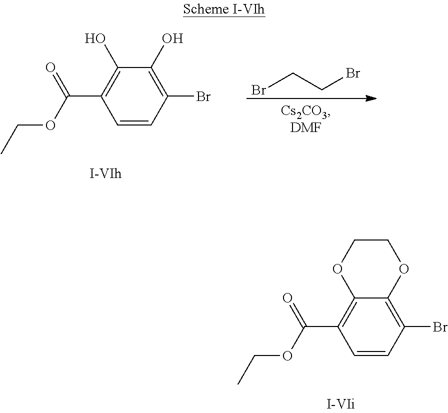

### General Procedure I-AV

To a solution of ethyl 4-bromo-2,3-dihydroxybenzoate (I-VIh) (3.5 g, 13.5 mmol) in DMF (25.0 mL) was added Cs₂CO₃ (9.7 g, 30.0 mmol) and the mixture was stirred at room temperature for 1 hour. 1.2-dibromoethane (3.1 g, 17.0 mmol) was added to the mixture and the mixture was stirred at 70° C. for 12 hours. The reaction mixture was diluted with ethyl acetate and washed with water and brine. The solvent was removed and the residue was purified by column chromatography on silica gel (eluent: PE:EtOAc=4:1) to give compound I-VIi (2.8 g, yield 71%) as a yellow solid. ¹H NMR (400 MHz, CDCl3) δ 7.39 (d, 1H), 7.11 (d, 1H), 4.34-4.25 (m, 6H), 1.31 (t, 3H).

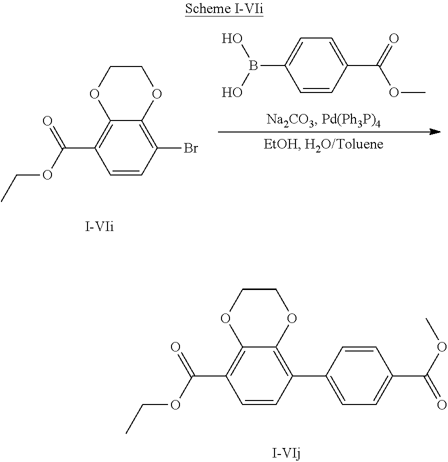

### General Procedure I-AW

To a solution of compound I-VIi (2.0 g, 7.0 mmol) in toluene (25.0 mL) were added EtOH (5.0 mL), aq. Na₂CO₃ solution (2.0 M, 4.0 mL), and 4-(methoxycarbonyl)phenylboronic acid and the mixture was stirred under nitrogen atmosphere for 10 minutes, then, Pd(Ph₃P)₄ (400 mg) was added and nitrogen was exchanged for three times. The mixture was stirred at 80° C. for 10 hours and cooled to room temperature. The reaction mixture was extracted with ethyl acetate and washed with water and brine. The solvent was removed and the residue was purified by column chromatography on silica gel using (eluent: PE:EtOAc=6:1) to afford compound I-VIj (1.5 g, yield 63%) as yellow solid. ¹H NMR (400 MHz, CDCl₃) δ 8.09 (d, 1H), 7.60 (d, 2H), 7.46 (d, 1H), 6.92 (d, 1H), 4.41-4.34 (m, 6H), 3.86 (s, 3H), 1.39 (t, 3H).

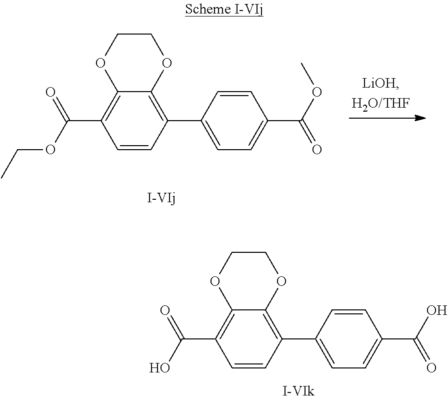

### General Procedure I-AX

To a solution of compound I-VIj (470 mg, 1.4 mmol) in THF (8.0 mL) was added aq. LiOH (2.0M, 5 mL, 10.0 mmol) and the mixture was stirred at room temperature for 17 hours. The solvent was removed and the pH value of the mixture was adjusted to 2 with 2.0 M HCl. The solid was collected by filtration and washed with water and dried to provide compound I-VIk (340 mg, yield 80%) as white solid. ¹H NMR (400 MHz, DMSO-d₆) δ 13.0 (brs, 2H), 8.05 (d, 2H), 7.71 (d, 2H), 7.37 (d, 1H), 7.01 (d, 1H), 4.35-4.41 (dt, 4H).

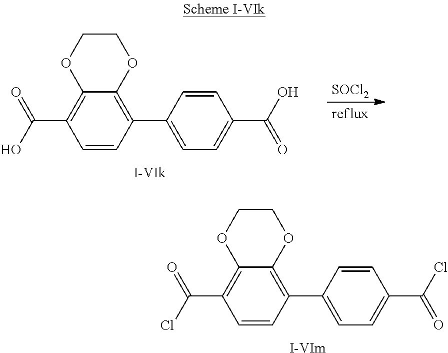

### General Procedure I-AY

A mixture of compound I-VIk (300 mg, 1.0 mmol) and SOCl₂ (5.0 mL) was refluxed for 2 hours. The excess SOCl₂ was removed under reduced pressure. The residue was co-evaporated with toluene (5 mL) for three times to afford compound I-VIm (336 mg, yield 99%) as yellow solid.

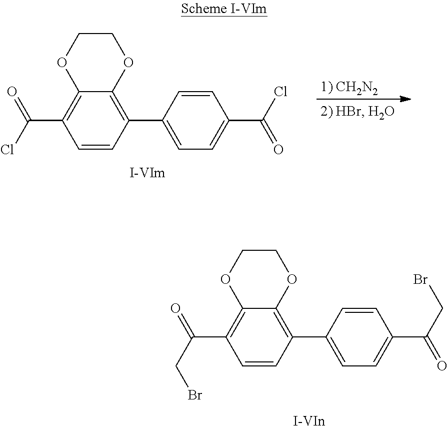

### General Procedure I-AZ

Compound I-VIm (336 mg, 1.0 mmol) was dissolved DCM (10.0 mL) and added dropwise at −10° C. to a solution of CH₂N₂ (1.0 M in diethyl ether, 6.0 mL, 6.0 mmol) in DCM (10.0 mL). After addition, the reaction mixture was stirred at 0° C. for 1 hour, then, 47% HBr aqueous solution (1 mL) was dropwise added to this solution at −10° C. and the mixture was stirred at the same temperature for 30 minutes. The mixture was warned to room temperature and stirred for another 30 minutes and diluted with ethyl acetate and washed with water, saturated NaHCO₃, and brine. The solvent was dried over anhydrous sodium sulfate and removed to provide compound I-VIn (210 mg, yield 46%) as yellow solid. ¹H NMR (400 MHz, CDCl₃) δ 8.02 (dd, 2H), 7.61 (dd, 2H), 7.43-7.41 (d, 1H), 6.92 (d, 1H), 4.53 (s, 2H), 4.42 (s, 2H), 4.38-4.36 (m, 2H), 4.29-4.27 (m, 2H).

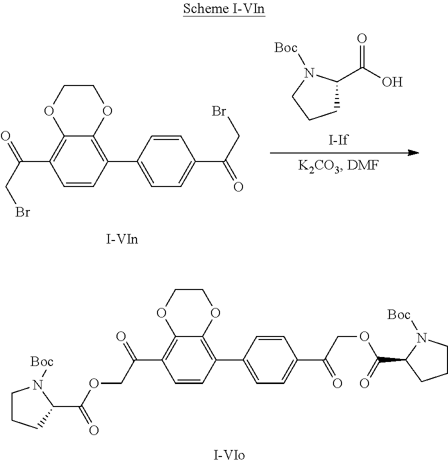

### General Procedure I-BA

To a solution of N-Boc-L-Proline (I-If) (430 mg, 2.0 mmol) in DMF (8.0 mL) was added potassium carbonate (276 mg, 2.0 mmol) and the mixture was stirred at room temperature for 2 hours. Compound I-VIn (180 mg, 0.40 mmol) in DMF (2.0 mL) was dropwise added to this mixture and the resulting mixture was stirred at room temperature for 12 hours. The mixture was diluted with ethyl acetate and washed with water and brine. The solvent was evaporated to provide compound I-VIo (150 mg, yield 52%) as yellow solid. MS (ESI) m/z (M+H)⁺ 723.3.

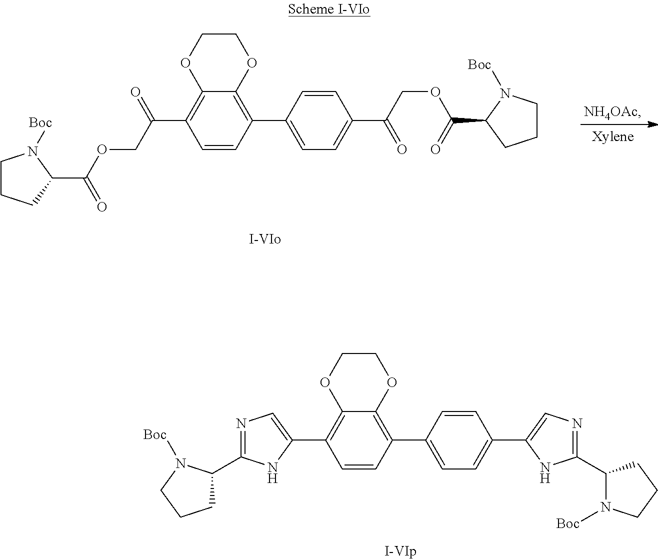

### General Procedure I-BB

To a solution of compound I-VIo (100 mg, 0.14 mmol) in xylene (10.0 mL) was added NH₄OAc (3.0 g, 40.0 mmol) and the mixture was refluxed for 16 hours. The reaction mixture was diluted with ethyl acetate and washed with water and brine. The solvent was removed and the residue was purified by column chromatography on silica gel to afford compound I-VIp (38 mg, yield 41%) as yellow solid. MS (ESI) m/z (M+H)⁺ 683.2.

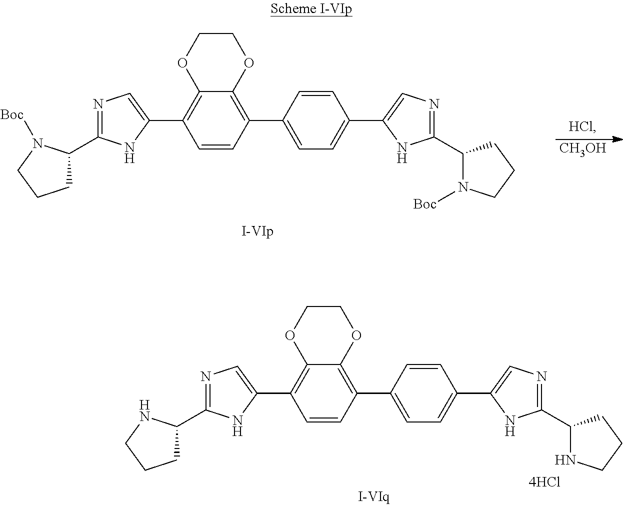

### General Procedure I-BC

To a solution of compound I-VIp (38 mg, 0.058 mmol) in methanol (3.0 mL) was added a solution of HCl in methanol (4.0 M, 2.0 mL, 8.0 mmol) and the mixture was stirred at room temperature for 4 hours. The solvent was removed to give compound I-VIq (33.7 mg, 96% yield) as yellow solid. MS (ESI) m/z (M+H)⁺ 483.

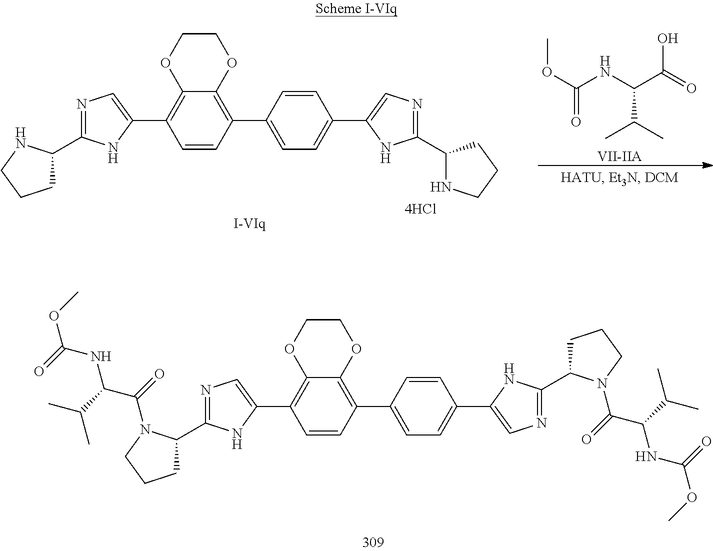

### General Procedure I-BD

To a suspension of compound I-VIq (32.5 mg, 0.05 mmol) in DCM (8.0 mL) was added triethylamine (202 mg, 2.0 mmol) and the mixture was stirred at room temperature for 1 hour, then, compound VII-IIA (18.0 mg, 0.11 mmol), HATU (41 mg, 0.11 mmol) was added and the mixture was stirred at room temperature for 12 hours. The mixture was diluted with DCM and washed with water and brine. The solvent was dried with sodium sulfate and removed to give crude product, which was purified by preparative HPLC to give compound 309 (9.1 mg, yield 22%) as white solid. MS (ESI) m/z (M+H)⁺ 797.2.

### Example I-VII

### Preparation of Compound 310

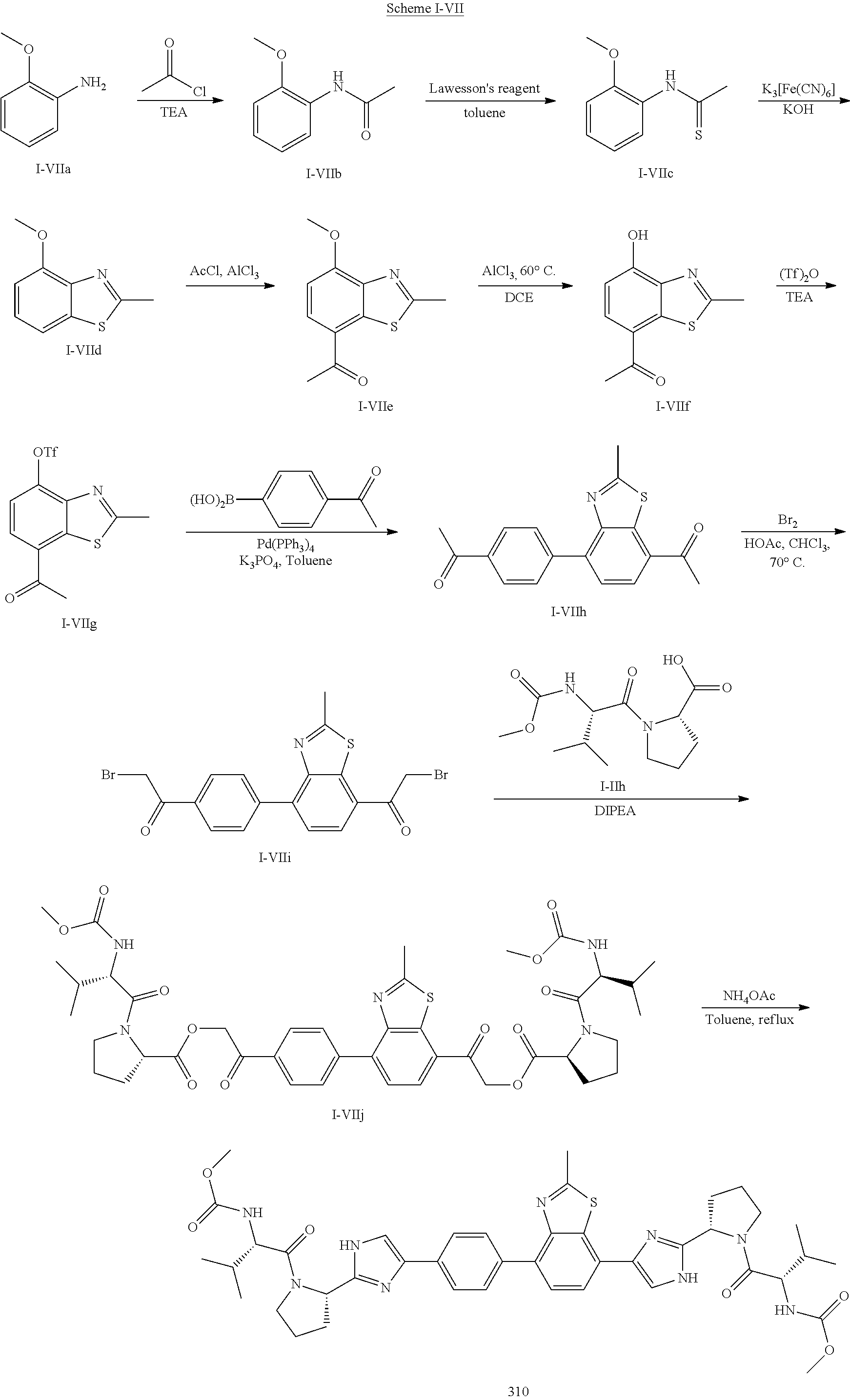

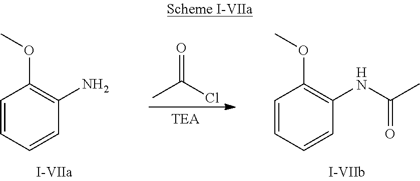

### General Procedure I-BE

2-Methoxybenzenamine (I-VIIa) (10 g, 81 mmol) was taken up in a 500 mL round bottom flask equipped with a liquid addition funnel and a guard tube; triethylamine (100 mmol, 10 g) was added to it in one lot. Above mixture was cooled to 0-5° C. and acetyl chloride (7.02 g, 90 mmol) was added dropwise maintaining temperature below 10° C. After addition, cooling bath was removed and the reaction mixture was stirred at r.t. for 3 hours. After the completion of reaction (TLC monitor), the reaction mixture was poured on ice-water, and aqueous layer was extracted with dichloromethane (300 mL×2). The combined extracts were washed with water, brine and dried over anhydrous magnesium sulfate. The volatiles were removed under the reduced pressure to obtain compound I-VIIb (12 g, yield 90%). MS (ESI) m/z (M+H)⁺ 166.

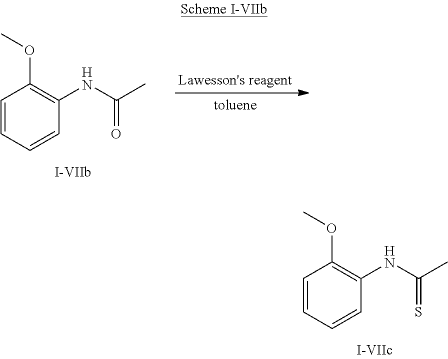

### General Procedure I-BF

A solution of compound I-VIIb (8.69 g, 52.6 mmol) and Lawesson's reagent (12.3 g, 30 mmol) in anhydrous toluene (200 mL) was stirred at 110° C. under argon for 3 hours. The reaction mixture was concentrated. The crude product was purified by flash chromatography on silica gel, eluting with petroleum ether/ethyl acetate, 9/1 to 7/3) to afford compound I-VIIc (9 g, yield 95%).

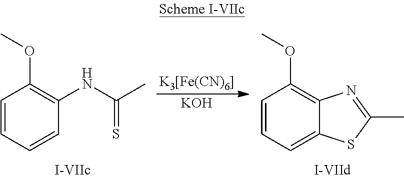

### General Procedure I-BG

Compound I-VIIc (9 g, 50 mmol) was diluted with ethanol (50 mL), and mixed with a solution of sodium hydroxide (14.4 g, 360 mmol) in water (35 mL). The resulting solution was added dropwise (over a period of 20 minutes) to a solution of potassium ferricyanide (53 g, 160 mmol) in water (15 mL) stirred at 90° C. The reaction mixture was kept stirring at 90° C. for 50 minutes after completion of the addition. The mixture was cooled to room temperature and filtered. The solid was rinsed with water, and then extracted with ethyl acetate. The aqueous layer was also extracted with ethyl acetate. Combined organic extracts were dried over sodium sulfate, filtered and evaporated. Purification by column chromatography on silica gel (petroleum ether/ethyl acetate=7/3) to provide compound I-VIId (2 g, yield 22%) as a solid. ¹H NMR (300 MHz, CDCl₃) δ 7.39 (d, J=7.8 Hz, 1H), 7.26 (t, 1H), 6.85 (d, J=8.1 Hz, 1H), 4.05 (s, 3H), 2.73 (s, 3H). MS (ESI) m/z (M+H)⁺ 180.2.

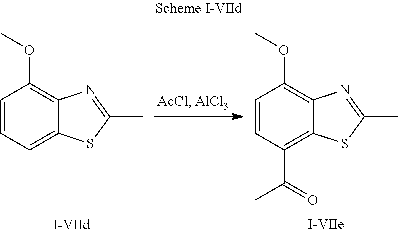

### General Procedure I-BH

A mixture of anhydrous AlCl₃ (1.85 g, 14 mmol) and compound I-VIId (1 g, 5.6 mmol) in carbon disulphide (10 mL) was heated under reflux for 1 hour. Acetyl chloride (0.5 g, 6.16 mmol) was added and heating was continued for 30 min before evaporation. The mixture was neutralized with aqueous sodium hydrogen carbonate and filtered, and the filtrate was continuously extracted with ethyl acetate. Then purified by column chromatography on silica gel (petroleum ether/ethyl acetate=5/1) to afford compound I-VIIe (0.3 g, yield 24%). ¹H NMR (400 MHz, CD₃OD) δ 7.92 (d, J=8.8 Hz, 1H), 6.91 (d, J=8.4 Hz, 1H), 4.06 (s, 3H), 2.80 (s, 3H), 2.62 (s, 3H). MS (ESI) m/z (M+H)⁺ 222.2.

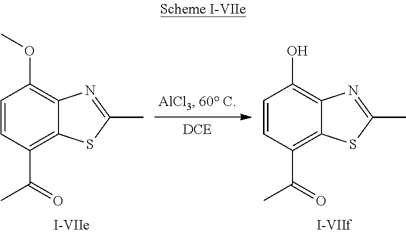

### General Procedure I-BI

To a solution of compound I-VIIe (200 mg, 0.9 mmol) in 1,2-dichloroethane (5 mL), AlCl₃ (180 mg, 1.35 mmol) was added under in nitrogen atmosphere. The reaction mixture was stirred at reflux for 5 hrs, the reaction mixture was poured into ice-water, then extracted with EtOAc (50 mL×3), the organic layer was washed with brine, dried over sodium sulfate and concentrated. The residue was purified by column chromatography on silica gel to give compound I-VIIf (120 mg, yield 64%). MS (ESI) m/z (M+H)⁺ 208.3.

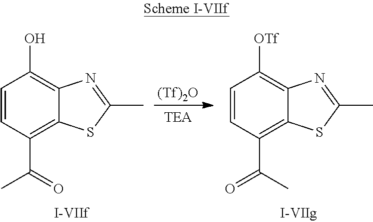

### General Procedure I-BJ

Compound I-VIIf (100 mg, 0.48 mmol) was dissolved in anhydrous CH₂Cl₂ (5 mL) in nitrogen atmosphere. Triethylamine (72 mg, 0.72 mmol) was added thereto by one portion. Then the mixture was cooled to 0° C., trifluoroacetic anhydride (125 mg, 0.6 mmol) was added portion-wise. The reaction mixture was stirred at 0° C. for 2 hrs, and then it was diluted with water, extracted with EtOAc (50 mL×3), the organic layer was washed with brine, dried over sodium sulfate and concentrated to provide compound I-VIIg, which was used directly for the next step.

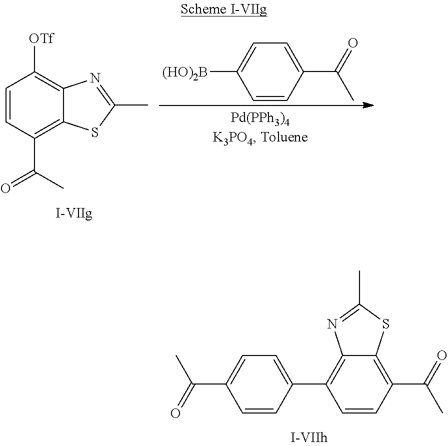

### General Procedure I-BK

To a solution of compound I-VIIg (120 mg, 0.35 mmol) in toluene (5 mL), Na₂CO₃ (53 mg, 0.5 mmol) and 4-acetylphenylboronic acid (82 mg, 0.4 mmol) were added, then keep the reaction in nitrogen atmosphere, then Pd(PPh₃)₄ (12 mg, 0.01 mmol) was added, the resulting mixture was stirred at 80° C. overnight, The reaction mixture was poured into water, extracted with EtOAc (50 mL×3). The combined organic layers were washed with brine, dried over sodium sulfate and concentrated. The residue was purified by column chromatography on silica gel (PE:EtOAc=2:1) to provide compound I-VIIh (100 mg, 83% yield over two steps). MS (ESI) m/z (M+H)⁺ 310.3.

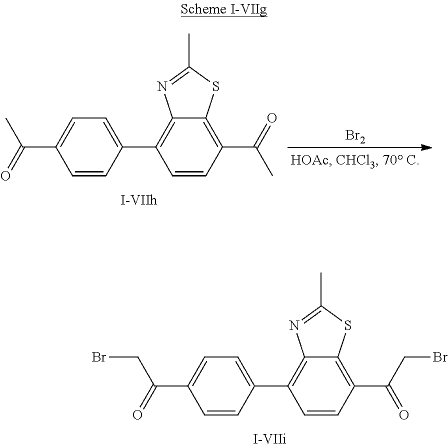

### General Procedure I-BL

Compound I-VIIh (100 mg, 0.32 mmol) was dissolved in CHCl₃ (2.5 mL) and acetic acid (2.5 mL), the mixture was stirred at 70° C., then bromine (202 mg, 1.28 mmol) was added dropwise. After the reaction was completed, the mixture was poured into water, extracted with EtOAc (50 mL×3). The combined organic layers were washed with brine, dried over sodium sulfate and concentrated. The product I-VIIi was used directly in the next step without further purification.

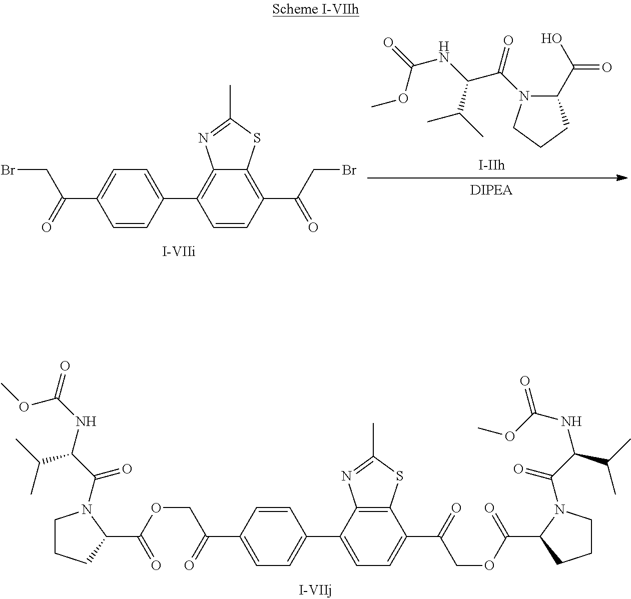

### General Procedure I-BM

Diisopropylethylamine (83 mg, 0.64 mmol) and compound I-VIIi (174 mg, 0.64 mmol) were added to a suspension of compound I-Ih (149 mg, 0.32 mmol) in THF (5 mL). The resulting mixture was stirred for 1 h as the solids dissolved. The reaction mixture was quenched by the addition of 13% aqueous sodium chloride (20 mL). The layers were separated, and the organic layer was concentrated, and purified by column chromatography on silica gel (PE:EtOAc=1:1) to obtain compound I-VIIj (20 mg, yield 8%). MS (ESI) m/z (M+H)⁺ 850.2.

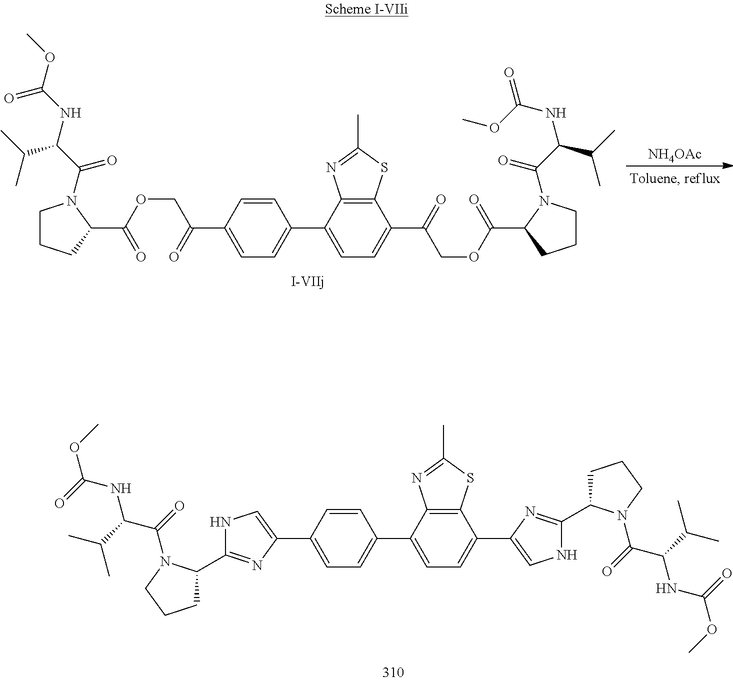

### General Procedure I-BN

To a solution of compound I-VIIj (20 mg, 0.024 mmol) in toluene (10 mL) was added ammonium acetate (5 g, 65 mmol) and heated to 100° C. overnight. LCMS indicated the reaction was complete, the mixture was cooled to r.t. and concentrated in vacuo. The residue was purified by Prep-HPLC to provide compound 310 (8 mg, yield 42%). MS (ESI) m/z (M+H)⁺ 810.7.

### Example I-VIII

### Preparation of Compound 311

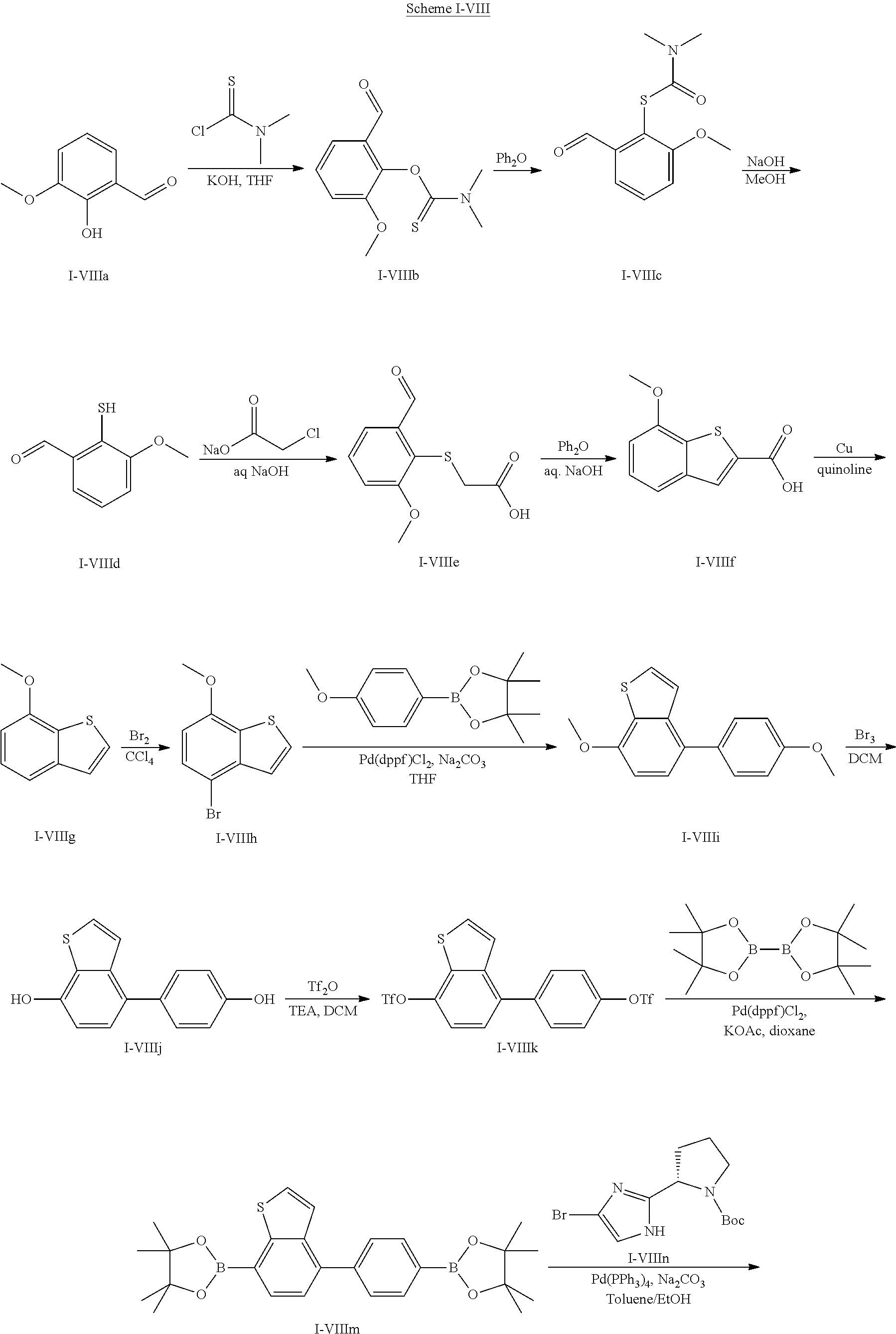

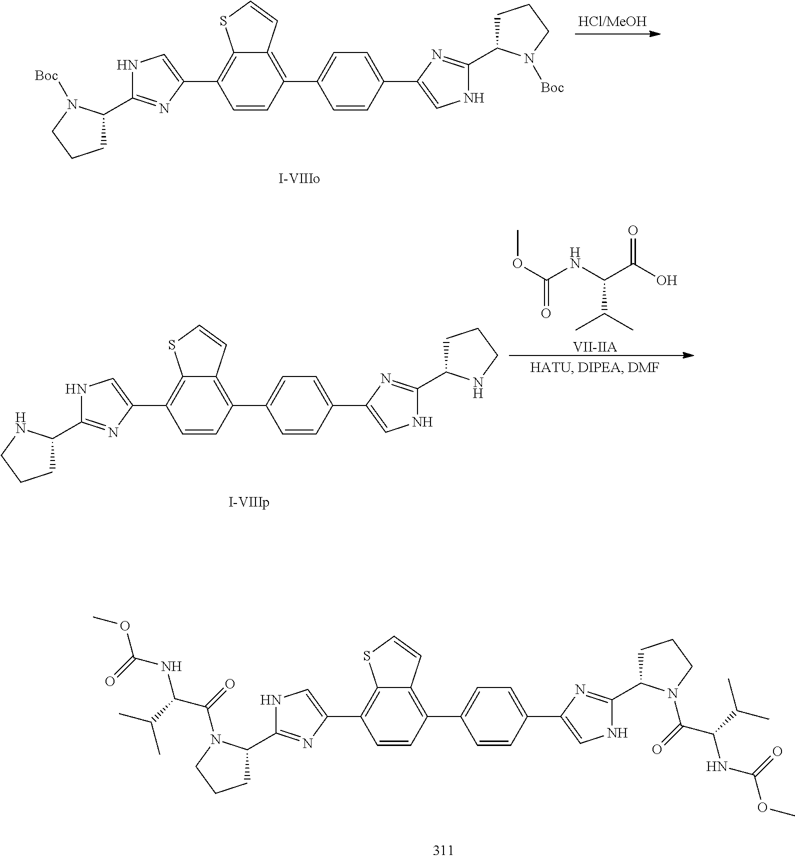

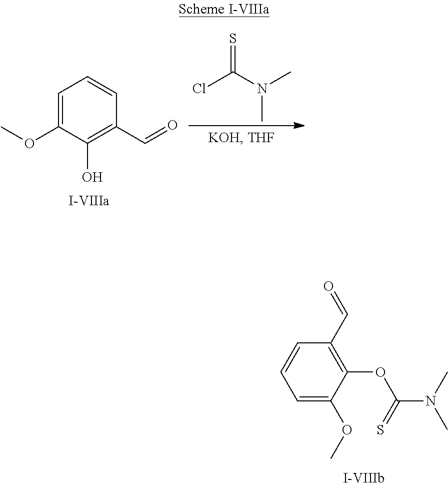

### General Procedure I-BO

To a mixture of 2-hydroxy-3-methoxybenzaldehyde (I-VIIIa) (24 g, 0.161 mmol) and KOH (11 g) in water (99 mL) was added a solution of N,N-dimethylthiocarbamoyl chloride (20 g, 0.161 mmol) in THF (44 mL) at 0° C. during 20˜30 min. The mixture was stirred for 10 min at room temperature, and then aqueous KOH (10%, 130 mL) was added. The mixture was extracted with EtOAc (100 mL×3), the combined organic layers were washed with brine, dried over Na₂SO₄, concentrated to give a residue, which was purified by column chromatography to provide compound I-VIIIb as a yellow solid (19 g, yield 95%). ¹H NMR (400 MHz, CDCl₃) δ 10.08 (s, 1H), 7.51-7.47 (m, 1H), 7.35-7.22 (m, 1H), 7.21-7.18 (m, 1H), 3.89 (m, 3H), 3.53-3.51 (m, 3H), 3.47-3.36 (m, 3H).

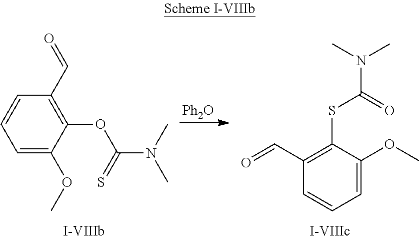

### General Procedure I-BP

A solution of compound I-VIIIb (18.6 g, 77.8 mmol) in diphenyl ether (120 mL) was stirred at 240-250° C. under nitrogen. TLC indicated the reaction was complete, then the mixture was cooled to r.t. Petroleum ether (500 mL) was added to the cooled solution, then the mixture was kept overnight at 0° C. The resulting brown solid was filtered off and crystallized from petroleum ether (b.p. 40-60° C.) to afford compound I-VIIIc as a yellow solid (9 g, yield 95%). ¹H NMR (300 MHz, CDCl₃) δ 10.03 (m, 1H), 8.70-8.39 (m, 1H), 7.44-7.06 (m, 2H), 3.96 (s, 3H), 3.44 (m, 3H), 3.38 (m, 3H).

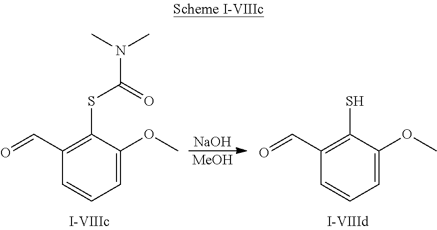

### General Procedure I-BQ

A solution of compound I-VIIIc (20 g, 0.083 mmol) in methanol (25 mL) was stirred under reflux under nitrogen for 2 hours with excess of aqueous 10% sodium hydroxide. The cooled mixture was washed with chloroform, and then acidified. Extraction with ether gave compound I-VIIId as yellow crystals (16 g, yield 80%).

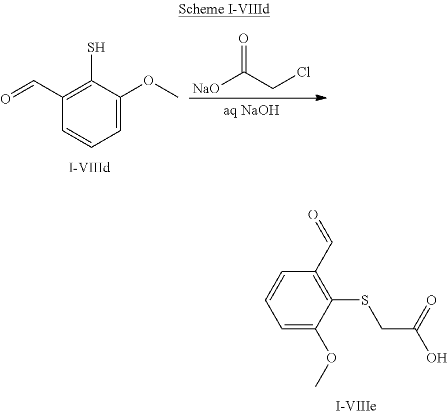

### General Procedure I-BR

A solution of compound I-VIIId (17 g, 0.101 mol) was kept at 100° C. for 4 hours with an excess of aqueous 10% sodium hydroxide. After the reaction was completed, the mixture was cooled to r.t., acidified with aq. HCl (2 N) to pH=4-5, the precipitate was collected and dried to afford acid I-VIIIe (13.6 g, yield 80%).

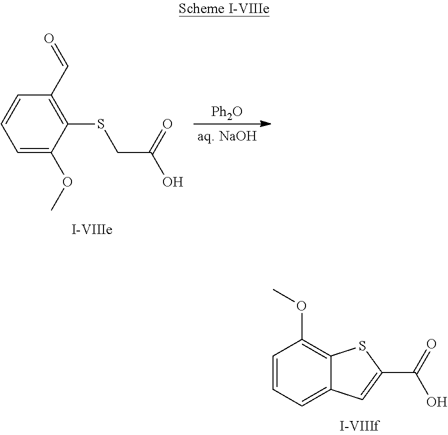

### General Procedure I-BS

A solution of acid I-VIIIe (13.6 g, 0.654 mmol) in diphenyl ether was vigorously stirred at 100° C. for 6 hours with aqueous 10% sodium hydroxide. After the reaction was completed, the mixture was cooled to r.t., the aqueous layer was separated and acidified with aq. HCl (2 N) to pH=4-5, the precipitate was collected and dried to afford acid I-VIIIf (8 g, yield 58%). ¹H NMR (300 MHz, CDCl₃) δ 8.09 (s, 1H), 7.74-7.64 (m, 1H), 7.52-7.13 (m, 1H), 6.98-6.86 (m, 1H), 4.28-4.03 (m, 3H).

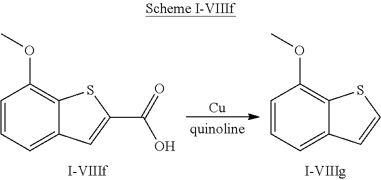

### General Procedure I-BT

A solution of compound I-VIIIf (10 g, 48.077 mmol), distilled quinoline (84 mL), and copper powder (4 g) was stirred vigorously at 210-220° C. for 1.5 hours under nitrogen, then cooled to 100° C., filtered, and poured into conc. HCl (360 mL). Neutral material was extracted into ether in the usual way to give, after work-up, an oil I-VIIIg (8 g, yield 100%).]

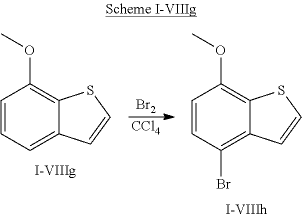

### General Procedure I-BU

A solution of bromine (7.8 g, 48.78 mmol) in dry tetrachloromethane (77 mL) was added dropwise during 1.5 hours to a stirred solution of I-VIIIg (8 g, 48.78 mmol) in tetrachloromethane (240 mL) at 0° C. After a further hour at 0° C., the organic layer was washed with water and brine, dried over Na₂SO₄, concentrated to give a residue, which was purified by column chromatography to provide compound I-VIIIh as a off white solid (8 g, yield 67%). ¹H NMR (400 MHz, CDCl₃) δ 7.51 (m, 3H), 6.67 (d, 1H), 4.05 (s, 3H).

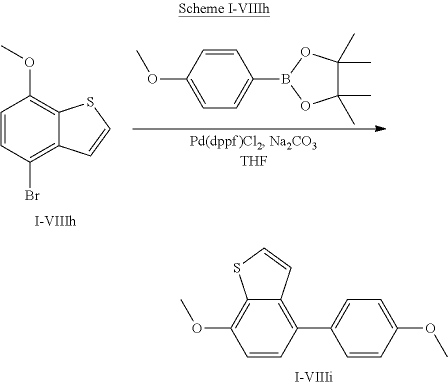

### General Procedure I-BV

To a solution of compound I-VIIIh (9.5 g, 39.095 mmol) in THF (165 mL) was added 4-methoxyphenylboronic acid, pinacol ester (7 g, 46.914 mmol), Na₂CO₃ (8.3 g, 78 mmol) and Pd(dppf)Cl₂ (1.5 g, catalyzed amount). The mixture was charged with N₂ for 5 minutes and heated to 80° C. overnight. LCMS detected that the reaction was complete. The mixture was diluted with water (200 mL) and extracted with EtOAc (150 mL×3). The combined organic layers were washed with brine, dried over Na₂SO₄, concentrated and the residue was purified by column chromatograph on silica gel (eluting with PE:EtOAc=20:1 to 10:1) to afford compound I-VIIIi (9.5 g, yield 90%) as a white solid. MS (ESI) m/z (M+H)⁺ 271.2.

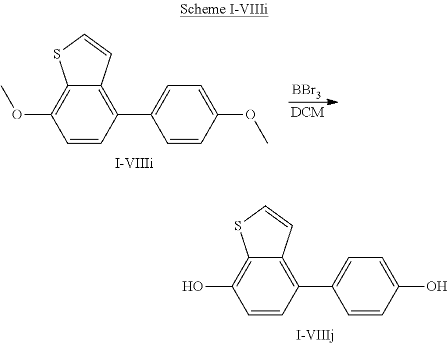

### General Procedure I-BW

To a solution of compound I-VIIIi (3 g, 11.11 mmol) in DCM (60 mL) was added BBr₃ (22.3 g, 0.0889 mmol) at −60 to −70° C. dropwise. After addition, the mixture was stirred at r.t. for 2 hours. The reaction mixture was poured into ice-water, extracted with EtOAc (80 mL×3). The organic layer was washed with brine, dried over Na₂SO₄, concentrated under reduced pressure to provide compound I-VIIIj. The crude product was used directly in the next step without further purification. MS (ESI) m/z (M+H)⁺ 243.3.

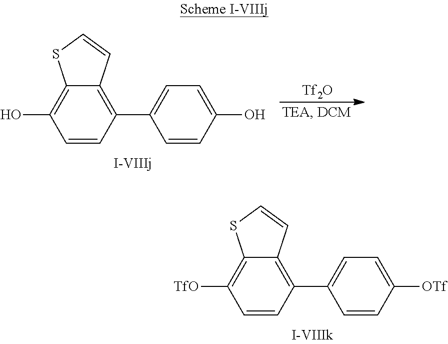

### General Procedure I-BX

To a solution of compound I-VIIIj (1.3 g, 4.815 mmol) in DCM (28 mL) was added triethylamine (2 g, 21.40 mmol) and (CF₃SO₂)₂O (3 g, 9.63 mmol) at −40° C. dropwise. The mixture was stirred at r.t for 3 hours. The reaction mixture was diluted with water (50 mL) and extracted by EtOAc (50 mL×3). The combined organic layers were dried over anhydrous Na₂SO₄ and concentrated. The crude product was purified by column chromatography on silica gel (eluting with PE:EtOAc=20:1 to 15:1) to give compound I-VIIIk (1.3 g, yield 48%) as a white solid.

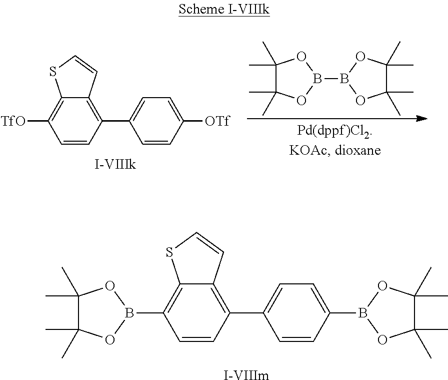

### General Procedure I-BY

To a solution of compound I-VIIIk (1.3 g, 2.57 mmol) in dioxane (38 mL) was added bis(pinacolato) diboron (2 g, 7.8 mmol), KOAc (1 g, 10.28 mmol) and Pd(dppf)Cl₂ (0.1 g, catalyzed amount). The mixture was purged with N₂ for 5 minutes and heated to 80° C. overnight. LCMS showed that the reaction was completed. The mixture was diluted with water (200 mL) and extracted with EtOAc (150 mL×3). The combined organic layers were washed with brine, dried over Na₂SO₄, concentrated and the residue was purified by column chromatography on silica gel (eluting with PE:EtOAc=20:1 to 15:1) to give compound I-VIIIm (0.6 g, yield 51%) as a white solid. ¹H NMR (400 MHz, CDCl₃) δ7.93 (m, 3H), 7.59 (d, 2H), 7.48 (m, 2H), 7.37 (d, 1H), 1.25 (s, 24H).

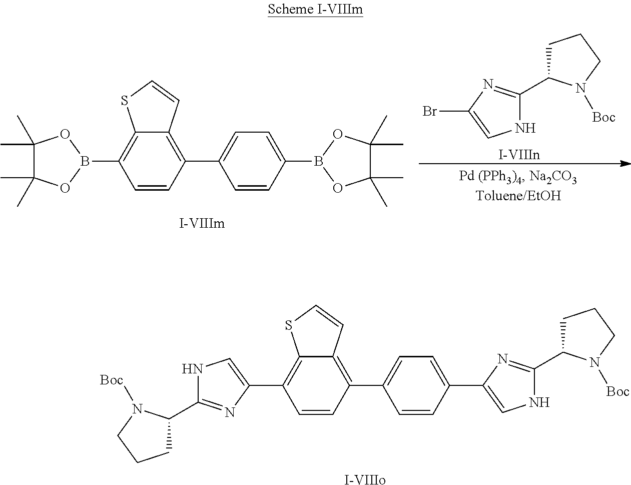

### General Procedure I-BZ

To a solution of compound I-VIIIm (0.6 g, 1.3 mmol) in toluene/EtOH (9 mL/1 mL) was added compound I-VIIIn (0.82 g, 2.6 mmol), Na₂CO₃ (550 mg, 5.2 mmol) and Pd(PPh₃)₄ (0.05 g, catalyzed amount). The mixture was charged with N₂ for 5 minutes and heated to 80° C. overnight. LCMS indicated the reaction was complete. The mixture was diluted with water (100 mL) and extracted with EtOAc (150 mL×3). The combined organic layers were washed with brine, dried over Na₂SO₄, concentrated and the residue the residue was purified by column chromatography on silica gel to give compound I-VIIIo (110 mg, yield 12%) as a white solid. MS (ESI) m/z (M+H)⁺ 681.3.

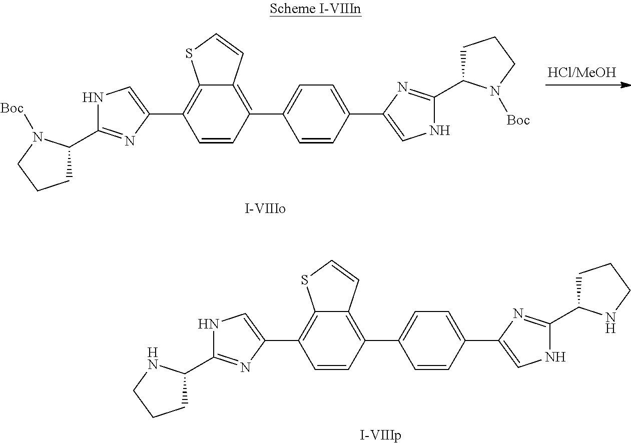

### General Procedure I-CA

A solution of compound I-VIIIo (110 mg, 0.16 mmol) in methanol (5 mL) was added a solution of HCl (4 M in methanol, 2.5 mL) and the mixture was stirred at r.t. overnight. LCMS detected the reaction was complete. The reaction solution was concentrated under reduced pressure to afford compound I-VIIIp as a white solid (80 mg, 100%). MS (ESI) m/z (M+H)⁺ 481.2.

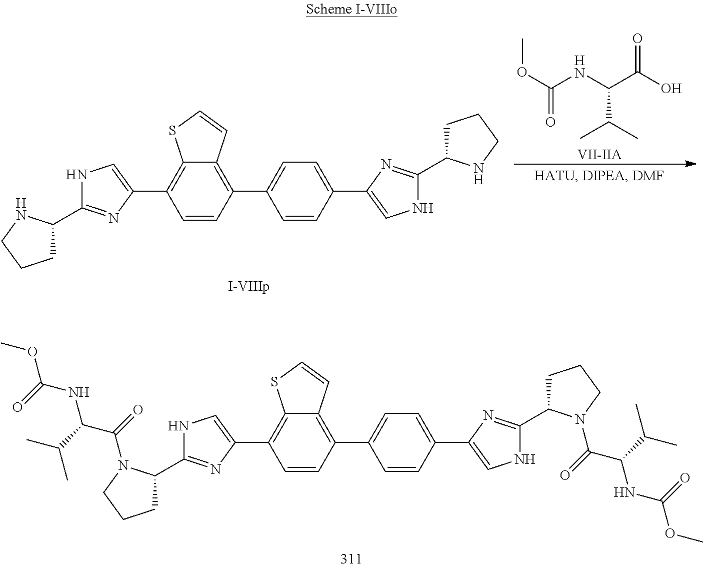

### General Procedure I-CB

To a solution of compound I-VIIIp (80 mg, 0.17 mmol) in anhydrous DCM (5 mL) was added compound VII-IIA (90 mg, 0.51 mmol), HATU (194 mg, 0.51 mmol) and DIPEA (220 mg, 1.7 mmol). The reaction solution was stirred at r.t. for 4 hours. The mixture was diluted with water (10 mL) and extracted with EtOAc (50 mL×3). The combined organic layers were washed with brine, dried over Na₂SO₄, concentrated and the residue was purified by Prep-HPLC to provide compound 311 (25 mg, yield 19%) as a white solid. MS (ESI) m/z (M+H)⁺ 795.5.

### Example I-IX

### Preparation of Compound 312

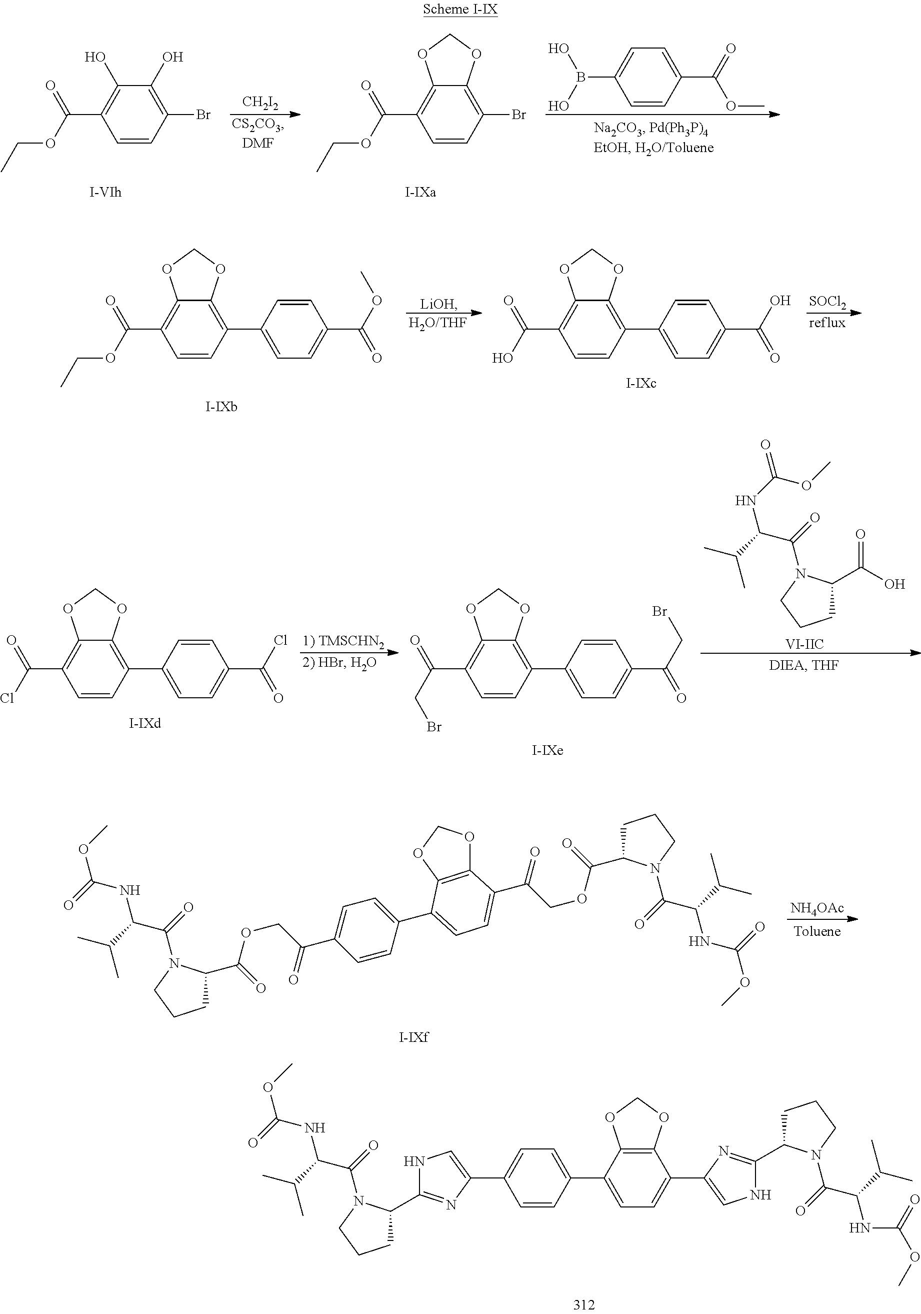

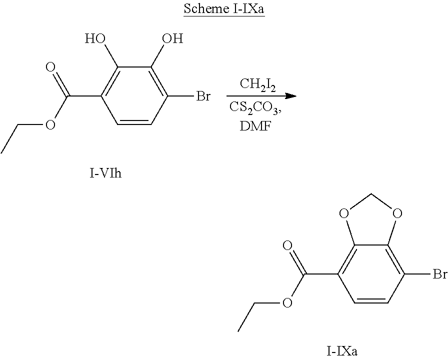

### General Procedure I-CC

To a solution of ethyl 4-bromo-2,3-dihydroxybenzoate (I-VIh, 1.3 g, 5.0 mmol) in DMF (10.0 mL) was added Cs₂CO₃ (3.5 g, 11.0 mmol) and the mixture was stirred at room temperature for 1 hour. CH₂I₂ (2.2 g, 8.1 mmol) was added to the mixture and the mixture was stirred at 70° C. for 12 hours. The reaction mixture was diluted with ethyl acetate and washed with water and brine. The solvent was removed and the residue was purified by column chromatography on silica gel using (eluent: PE:EtOAc=4:1) to provide compound I-IXa (700 mg, yield 52%) as yellow solid. ¹H NMR (400 MHz, CDCl3) δ 7.31 (d, 1H), 7.00 (d, 1H), 6.15 (s, 2H), 4.32 (q, 2H), 1.30 (t, 3H)

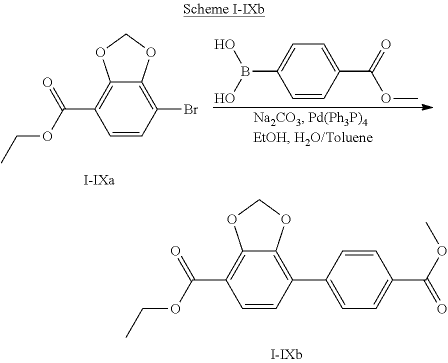

### General Procedure I-CD

To a solution of compound I-IXa (700 mg, 2.6 mmol) in toluene (15.0 mL) were added EtOH (3.0 mL), aq. Na₂CO₃ (2.0 M, 1.5 mL) and 4-(methoxycarbonyl)phenylboronic acid, the mixture was stirred under nitrogen atmosphere for 10 minutes, then, Pd(Ph₃P)₄ (90 mg, 0.08 mmol) was added and the flask was purged with nitrogen for three times. The mixture was stirred at 80° C. for 10 hours. After cooling to room temperature, the reaction mixture was extracted with ethyl acetate and washed with water and brine. The solvent was removed and the residue was purified by column chromatography on silica gel (eluent: PE:EtOAc=6:1) to give compound I-IXb (560 mg, yield 59%) as yellow solid. ¹H NMR (400 MHz, CDCl₃) δ 8.09 (d, 2H), 7.60 (d, 2H), 7.46 (d, 1H), 6.92 (d, 1H), 6.15 (s, 2H), 3.86 (q, 3H), 1.38 (t, 3H).

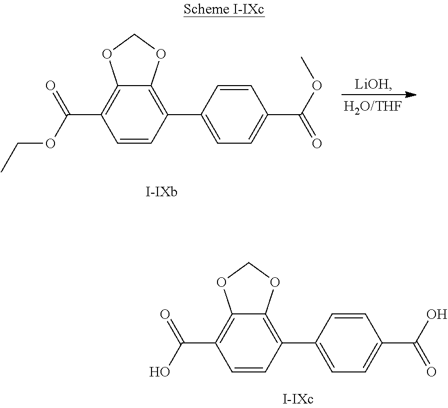

### General Procedure I-CE

To a solution of compound I-IXb (560 mg, 1.7 mmol) in THF (10.0 mL) was added a solution of LiOH in water (2.0M, 8.0 mL, 16.0 mmol) and the mixture was stirred at room temperature for 17 hours. The solvent was removed and the pH value of the mixture was adjusted to 2 with aq. HCl (2.0 M). The solid was collected by filtration and washed with water and dried to give compound I-IXc (460 mg, yield 95%) as white solid.

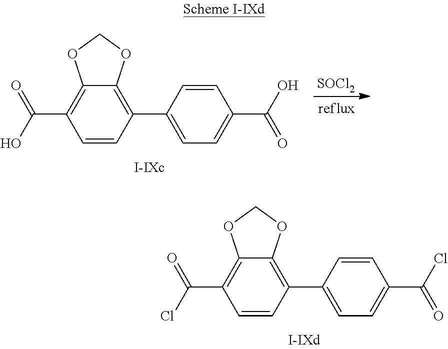

### General Procedure I-CF

A mixture of compound I-IXc (350 mg, 1.2 mmol) and SOCl₂ (5.0 mL) was refluxed for 2 hours. The excess SOCl₂ was removed under reduced pressure. The residue was co-evaporated with toluene (5 mL) for three times to afford compound I-IXd (358 mg, 93% yield) as yellow solid.

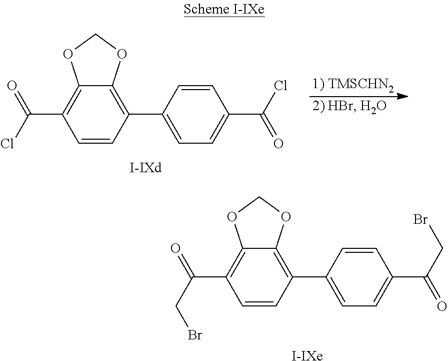

### General Procedure I-CG

Compound I-IXd (353 mg, 1.1 mmol) was dissolved in anhydrous DCM (10 mL) and added dropwise at −10° C. to a solution of TMSCH₂N₂ (2.0 M, 4.0 mL, 8.0 mmol) in anhydrous DCM (4.0 mL). After addition, the reaction mixture was stirred at 0° C. for 1 hour, then, aqueous HBr (47%) (4.0 mL) was dropwise added to this solution at −10° C. and the mixture was stirred at the same temperature for 30 minutes. The mixture was warmed to room temperature and stirred for another 30 minutes and diluted with ethyl acetate and washed with water, saturated aq. NaHCO₃, and brine. The solvent was dried over anhydrous sodium sulfate and removed to give compound I-IXe (370 mg, yield 74%) as yellow solid. ¹H NMR (400 MHz, CDCl₃) δ 8.05-8.03 (dd, 2H), 7.64-7.62 (dd, 2H), 7.44 (d, 1H), 6.94 (d, 1H), 6.16 (s, 2H), 4.58 (s, 2H), 4.46 (s, 2H).

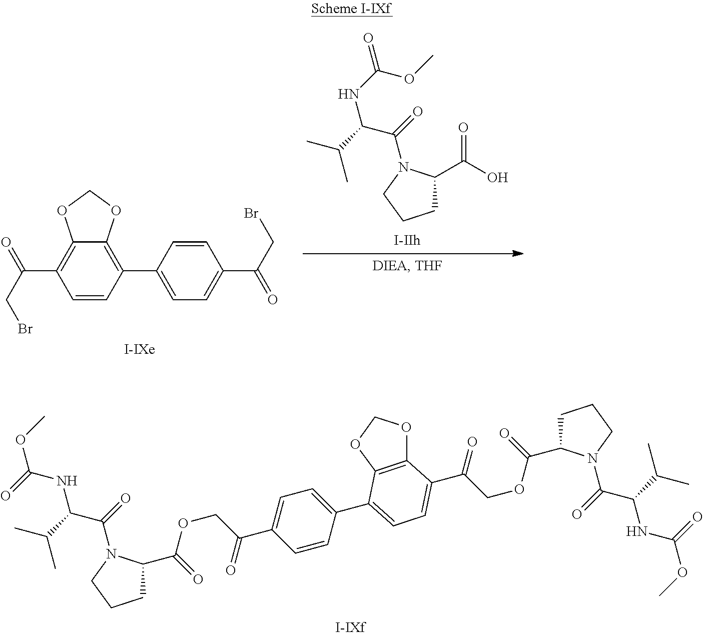

### General Procedure I-CH

To a mixture of compound I-IIh (546 mg, 2.0 mmol) and compound I-IXe (350 mg, 0.78 mmol) in THF (8.0 mL) was added dropwise DIEA (520 mg, 4.0 mmol) and the mixture was stirred at room temperature for 12 hours. After the reaction was completed, the mixture was diluted with ethyl acetate and washed with aq. HCl (1.0 M), water and brine. The solvent was removed and the residue was purified by column chromatography on silica gel (eluent: DCM:Methanol=15:1) to give compound I-IXf (210 mg, yield 41%) as yellow solid. MS (ESI) m/z (M+H)⁺ 823.

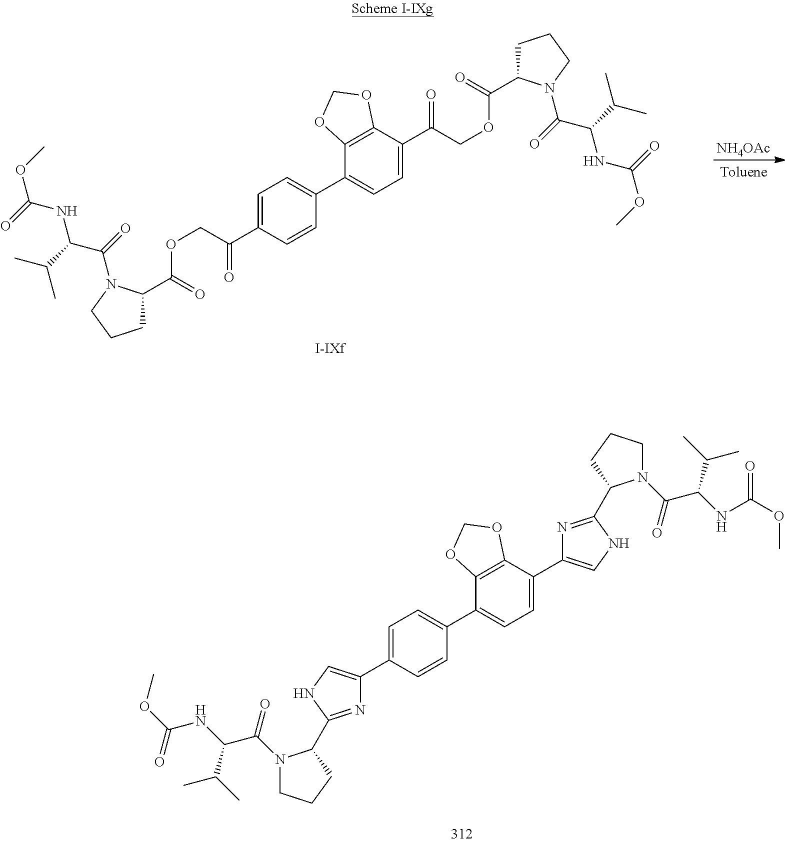

### General Procedure I-CI

To a solution of compound I-IXf (250 mg, 0.31 mmol) in toluene (10.0 mL) was added NH₄OAc (4.0 g, 50 mmol) and the mixture was refluxed for 16 hours. The reaction mixture was diluted with ethyl acetate and washed with water and brine. The solvent was removed and the residue was purified by preparative HPLC to give 312 (43.5 mg, yield 20%) as white solid. MS (ESI) m/z (M+H)⁺ 783.4.

### Example I-X

### Preparation of Compound 313

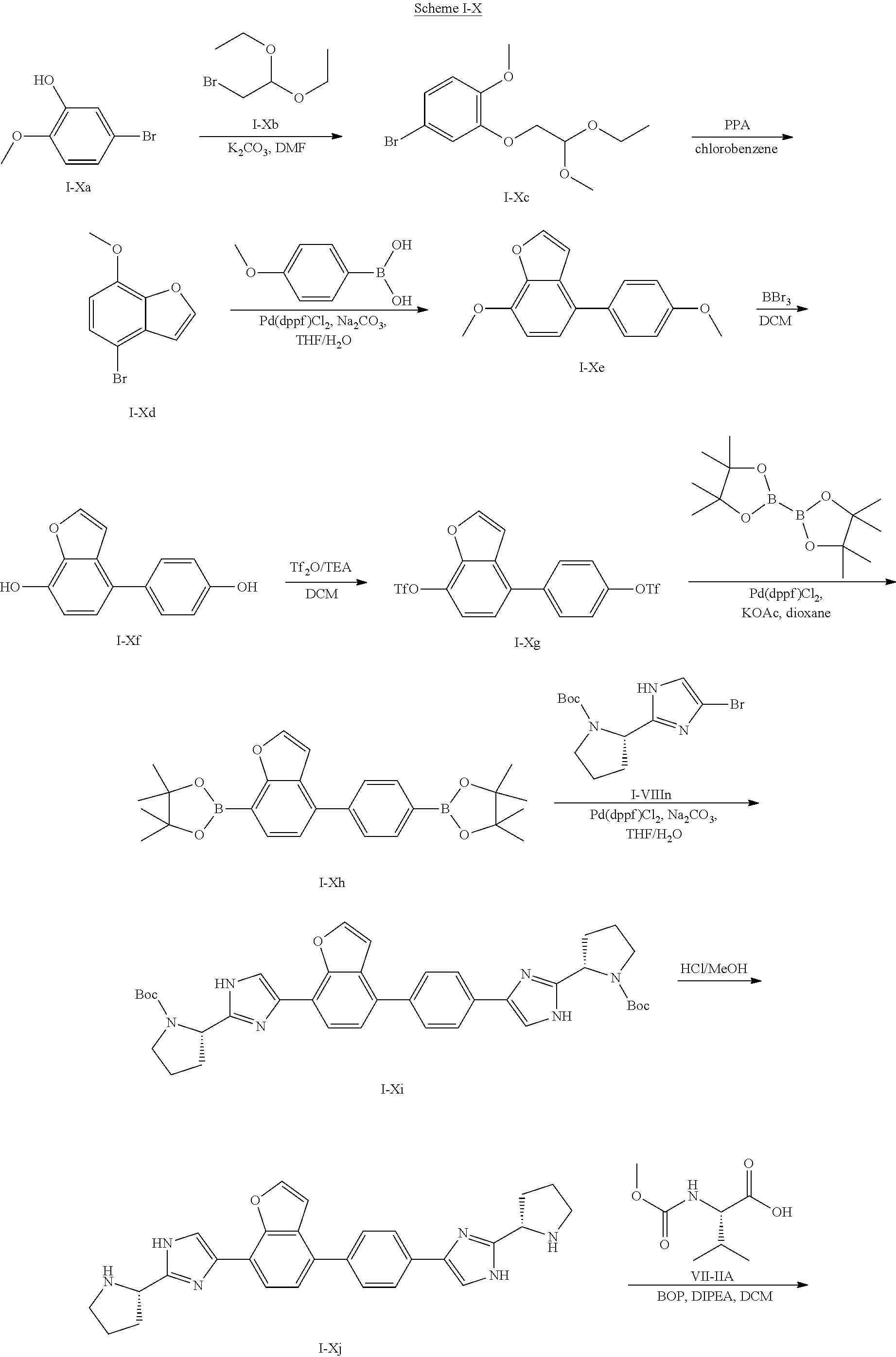

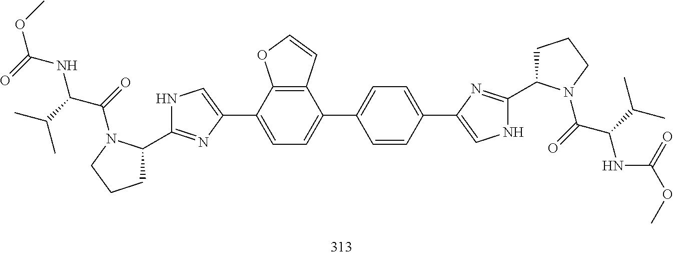

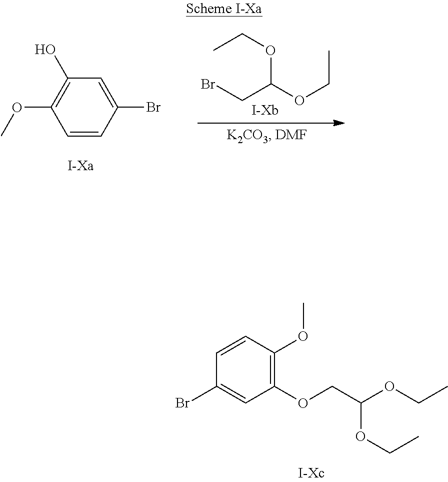

### General Procedure I-CJ

A mixture of 5-bromo-2-methoxyphenol (I-Xa, 10 g, 49.3 mmol) K₂CO₃ (6.8 g, 49.3 mmol) and bromoacetaldehyde diethyl acetal (I-Xb, 9.7 g, 49.3 mmol) in 200 mL of DMF was stirred at 140° C. for 16 h. The reaction mixture was then cooled to room temperature and diluted with 80 mL of 2N sodium hydroxide followed by 400 mL ethyl acetate. The organic layers was separated, washed with water (200 mL), brine (200 mL), dried over Na₂SO₄, and concentrated under reduced pressure to provide compound I-Xc (15 g, yield 96%), which was used directly in the next step.

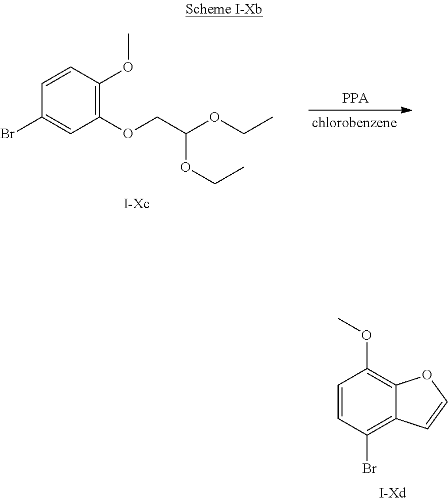

### General Procedure I-CK

To a mixture of PPA (1.7 g) in chlorobenzene (50 mL) was added a solution of compound I-Xc (1.6 g, 5.0 mmol) in 10 mL chlorobenzene dropwise at 80° C. The resulting mixture was stirred for 1 h at 120° C. The reaction mixture was cooled room temperature and chlorobenzene was decanted from PPA phase. The remaining residue was washed with MTBE (5×30 mL). All of the organic phase was combined and concentrated under reduced pressure to provide a dark amber oil. This oil was purified by silica gel chromatography (eluting with PE:EA=100:1) to give compound I-Xd (0.5 g, yield: 44%). ¹H NMR (300 MHz, CDCl₃): δ7.65-7.64 (d, 1H), 7.29-7.25 (d, 1H), 6.78-6.77 (d, 1H), 6.68-6.65 (d, 1H), 3.97 (s, 3H).

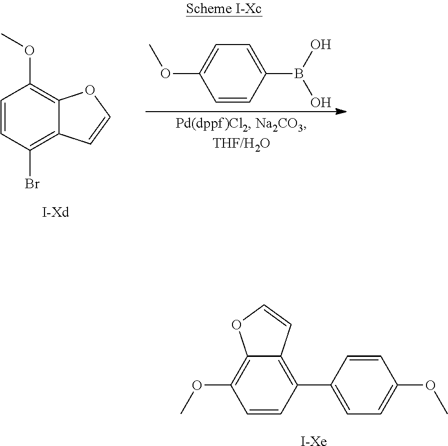

### General Procedure I-CL

A mixture of compound I-Xd (1 g, 4.42 mmol), 4-methoxyphenylboronic acid, pinacol ester (0.67 g, 4.42 mmol), Na₂CO₃ (1.87 g, 17.7 mmol), and Pd(dppf)Cl₂ (0.32 g, 0.44 mmol) in THF/H₂O (25 mL/5 mL) was stirred at 80° C. overnight. After concentrated under reduced pressure and the residue was diluted with water, and extracted with EtOAc. The organic layer was separated, dried over Na₂SO₄ and concentrated under reduced pressure. The residue was purified by column chromatography (eluting with PE:EA=100:1) to afford compound I-Xe (0.9 g, yield: 80%).

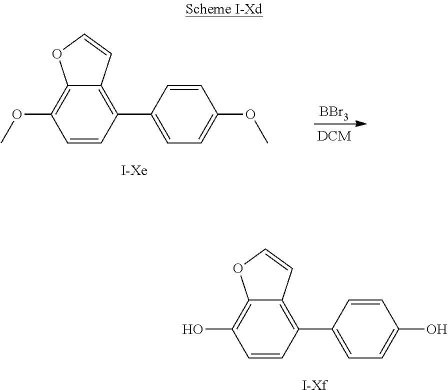

### General Procedure I-CM

To a solution of compound I-Xe (1 g, 3.9 mmol) in dry DCM (40 mL) was added BBr₃ (5.88 g, 23.5 mmol) slowly at −70° C. Then the reaction mixture was allowed to warm to room temperature and stirred for 1 h. The mixture was quenched with 20 mL ice-water and extracted with EtOAc (3×50 mL). The combined organic layers were washed with brine, dried over Na₂SO₄. Then concentrate to give compound I-Xf (550 mg, yield: 62%), which was used for next step without further purification.

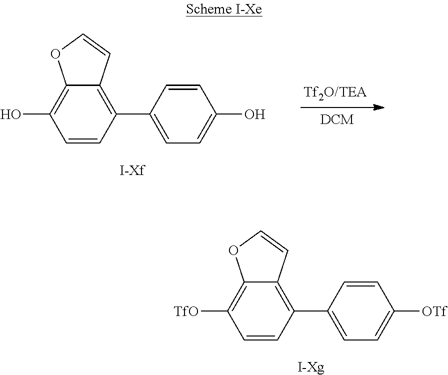

### General Procedure I-CN

To a solution of compound I-Xf (550 mg, 2.43 mmol) and TEA (1.35 mL, 9.72 mmol) in 40 mL of DCM was added Tf₂O (0.98 mL, 5.84 mmol) dropwise at −20° C. The reaction mixture was stirred for 10 min. at −20° C., then 30 min. at ambient temperature. After quenched with 30 mL ice-water (5 mL), the mixture extracted with DCM (20 mL), washed with brine (10 mL), dried over Na₂SO₄ and concentrate under reduced pressure. The residue was purified by column chromatography (eluting with PE:EA=100:1) to give compound I-Xg (1 g, yield: 83%).

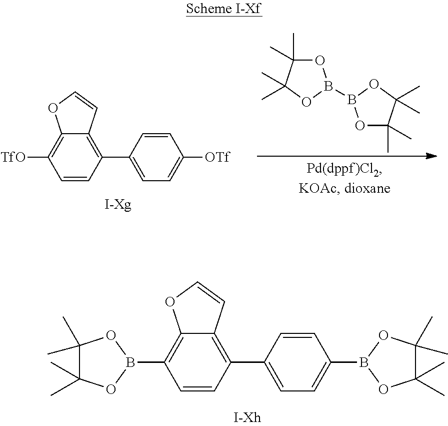

### General Procedure I-CO

A mixture of compound I-Xg (1 g, 2 mmol), bis(pinacolato) diboron (1.24 g, 4.9 mmol), Pd(dppf)Cl₂ (0.15 g 0.2 mmol) and KOAc (0.4 g, 4 mmol) in 30 mL of dioxane was stirred at reflux overnight. Then it was concentrated and the residue was diluted with brine (10 mL), extracted with DCM (3×50 mL). The combined organic layers were dried over Na₂SO₄ and concentrate under reduced pressure. The residue was purified by column chromatography (eluting with PE:EA=100:1) to give compound I-Xh (0.75 g, yield 83%). MS (ESI) m/z (M+H)⁺ 445.8.

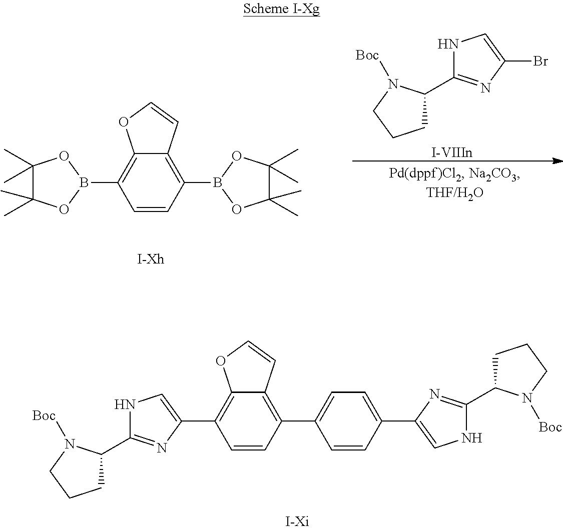

### General Procedure I-CP

A mixture of compound I-Xh (210 mg, 0.47 mmol), compound I-VIIIn (300 mg, 0.95 mmol), Na₂CO₃ (200 mg, 1.88 mmol) and Pd(dppf)Cl₂ (34 mg, 0.047 mmol) in THF/H₂O (25 mL/5 mL) was stirred at reflux overnight. After concentrated under reduced pressure, the residue was diluted water, extracted with EtOAc. The organic layer was separated, dried over Na₂SO₄ and concentrated under reduced pressure. The residue was purified by Prep. TLC to give compound I-Xi (100 mg, yield: 33%). MS (ESI) m/z (M+H)⁺ 665.3.

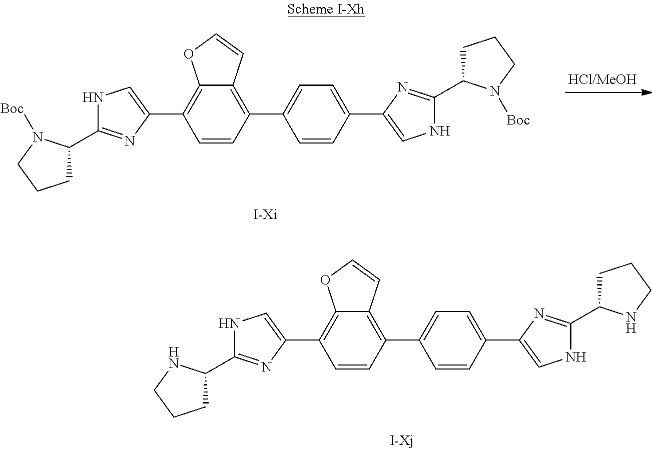

### General Procedure I-CQ

A mixture of compound I-Xi (100 mg, 0.15 mmol) in 30 mL of 4M HCl/MeOH was stirred at room temperature for 1 h. Then the mixture was concentrate under reduced pressure to give compound I-Xj, which was used for next step without further purification.

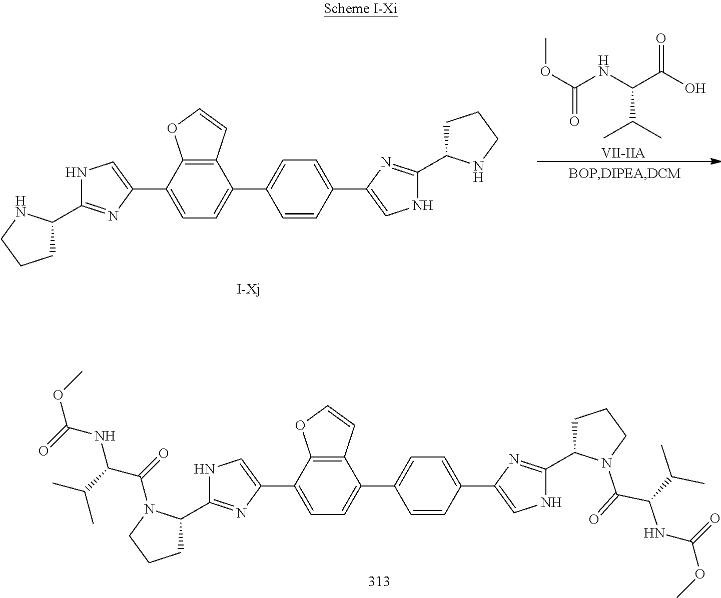

### General Procedure I-CR

A mixture of compound I-Xj (100 mg, 0.22 mmol), compound VII-IIA (90 mg, 0.52 mmol) and DMA (111 mg, 0.86 mmol) in 20 mL of DCM was stirred at room temperature for 15 min. (Benzotriazol-1-yloxy)tris(dimethylamino)phosphonium hexafluorophosphate (BOP, 114 mg, 0.26 mmol) was then added to the stirring mixture. The reaction mixture was stirred at room temperature overnight. Then the mixture was diluted with water (10 mL) and extracted with DCM (3×10 mL). The combined organic layers were separated, dried over Na₂SO₄ and concentrate under reduced pressure. The residue was purified by Prep. HPLC to afford compound 313 (9 mg, yield: 5.3%). ¹H NMR (400 MHz, CD₃OD) δ7.944 (s, 1H), 7.89-7.80 (m, 3H), 7.72-7.67 (m, 3H), 7.42-7.36 (m, 2H), 7.10-7.09 (s, 1H), 5.36-5.28 (m, 1H), 5.25-5.19 (m, 1H), 4.28-4.26 (m, 2H), 4.14-4.02 (m, 2H), 3.91-3.83 (m, 2H), 3.67-3.61 (s, 6H), 2.40-2.20 (m, 5H), 2.17-1.98 (m, 5H), 1.02-0.98 (m, 12H), MS (ESI) m/z (M+H)⁺ 779.4.

### Example I-XI

### Preparation of Compound 314

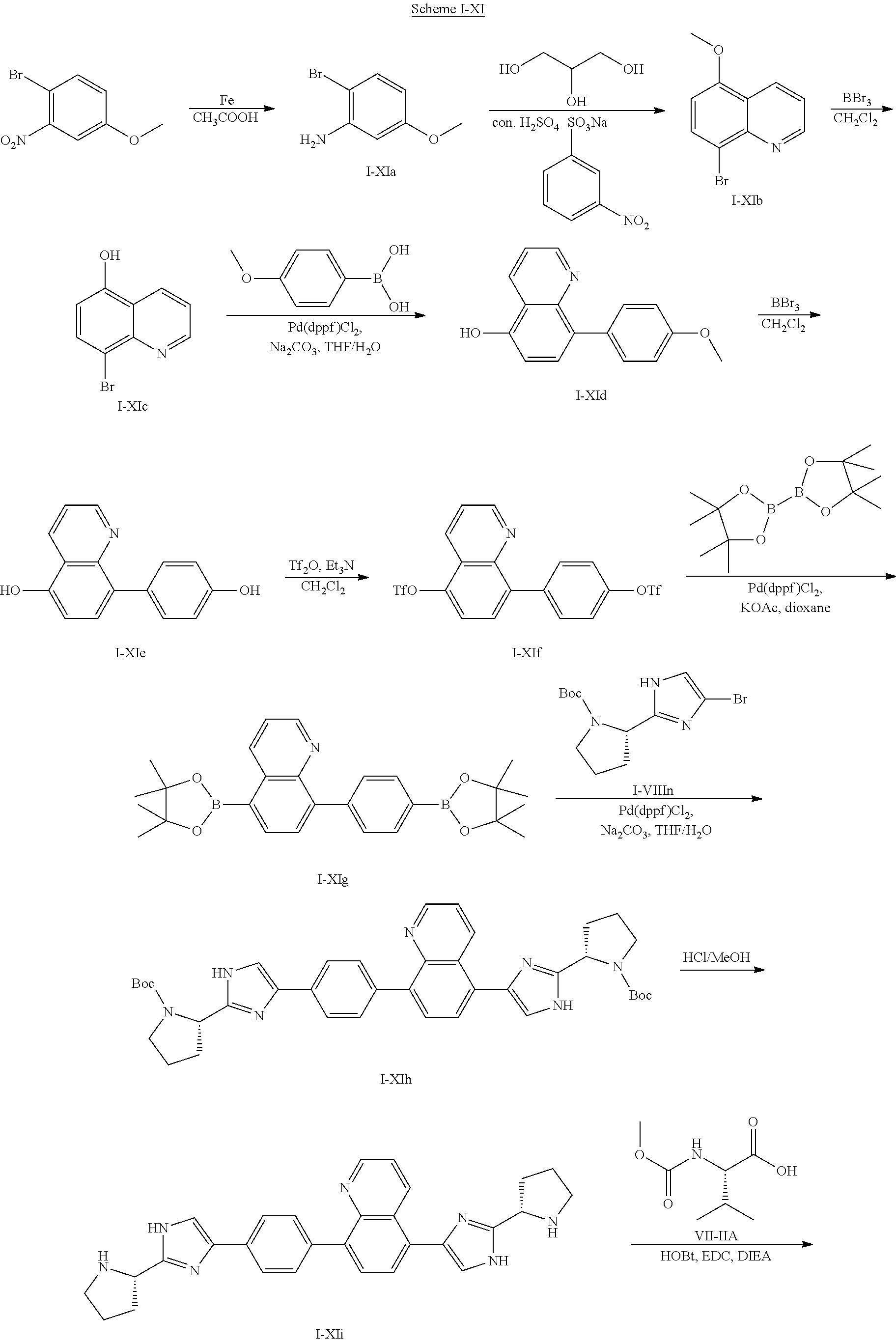

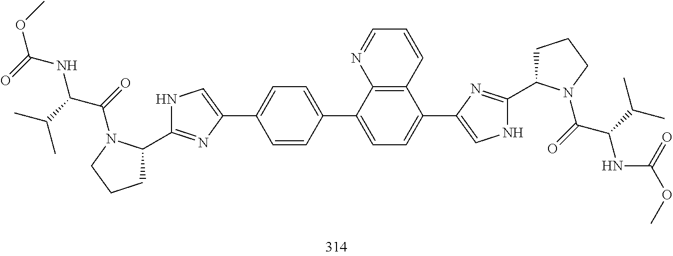

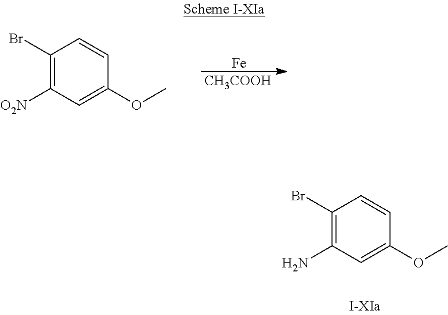

### General Procedure I-CS

A mixture of 4-bromo-3-nitroanisole (5 g, 21.6 mmol) and Fe (9.7 g, 0.17 mol) in 30 mL of acetic acid was stirred at r.t for 2 h. After removal of the solvent under reduced pressure, the brown residue was taken up in 100 mL of water and treated with 10% of aq. K₂CO₃ until pH 10. The mixture was extracted with EtOAc (150 mL×2) and the combined organic extracts were separated, dried over MgSO₄, and concentrated to afford compound I-XIa (3 g, yield: 52%). MS (ESI) m/z (M+H)⁺ 203.

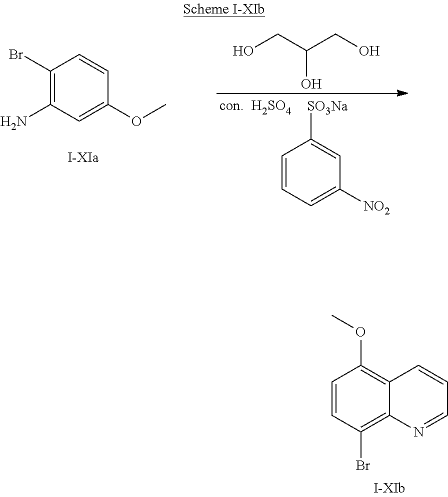

### General Procedure I-CT

3-Nitrobenzenesulfonic acid sodium salt (3.3 g, 15 mmol) was added to the mixture of compound I-XIa (3 g, 15 mmol) and propane-1,2,3-triol (3.6 g, 0.039 mol). Then 12 mL of conc. H₂SO₄ was added, and the reaction mixture was stirred at 140° C. for 3 h under N₂ protection. After cooled to r.t, water (18 g) was added, and a grayish by product was filtered off. The filtrate was diluted with aq. NaOH (20 mL, 50%) and extracted with of CH₂Cl₂ (80 mL). The organic layer was separated, washed with brine (20 mL), dried over MgSO₄, and concentrated. The residue was purified by column chromatography to afford compound I-XIb (600 mg, yield: 19%). MS (ESI) m/z (M+H)⁺ 238.

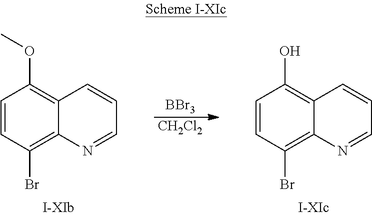

### General Procedure I-CU

BBr₃ (1.3 g, 5.2 mmol) was added dropwise to the mixture of compound I-XIb (600 mg, 2.6 mmol) in 10 mL of anhydrous CH₂Cl₂ at −78° C. After addition, the reaction mixture was warmed to r.t. and stirred for 5 h. Then water (10 mL) was added, and extracted with EtOAc (100 mL×3), the organic layers was separated, dried, and concentrated under reduced pressure. The residue was purified by column chromatography to give compound I-XIc (60 mg, yield: 11%). MS (ESI) m/z (M+H)⁺ 223.

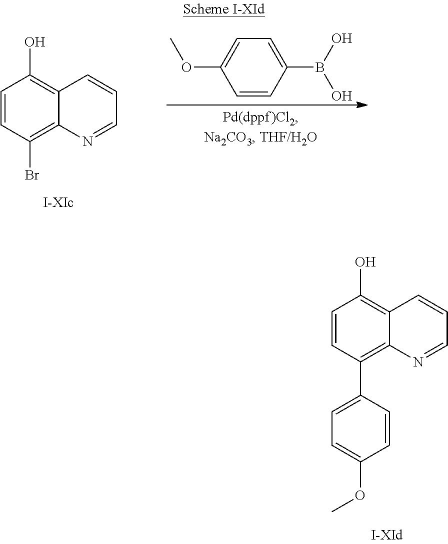

### General Procedure I-CV

A mixture of compound I-XIc (2 g, 8 mmol), 4-methoxy-phenyl boronic acid (1.3 g, 8 mmol), Pd(dppf)₂Cl₂ (0.3 g, 0.5 mmol) and Na₂CO₃ (1.8 g, 16 mmol) in THF/H₂O (36 mL/4 mL) was stirred at 80° C. overnight. After concentration under reduced pressure, the residue was diluted with water, and extracted with EtOAc. The organic layer was separated, dried over Na₂SO₄ and concentrated under reduced pressure. The residue was purified by column chromatography (eluting with PE:EA=6:1) to afford compound I-XId (2.8 g, yield: 62.2%).

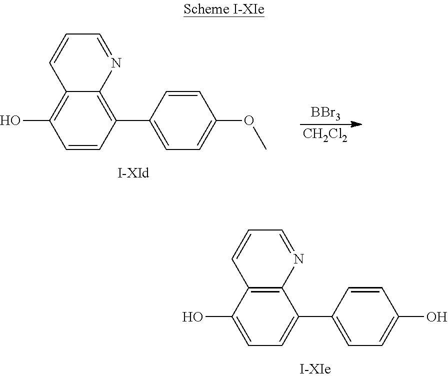

### General Procedure I-CW

BBr₃ (1.8 g, 7.16 mmoL) was added dropwise to the mixture of compound I-XId (900 mg, 3.58 mmoL) in 10 mL of anhydrous CH₂Cl₂ at −78° C. After addition, the reaction mixture was warmed to r.t. and stirred for 5 h. Then water (10 mL) was added, and extracted with EtOAc (100 mL×3), the organic layers were separated, dried, and concentrated under reduced pressure. The residue was purified by column chromatography (DCM/MeOH=8/1) to afford compound I-XIe (600 mg, yield: 71%). MS (ESI) m/z (M+H)⁺ 238.

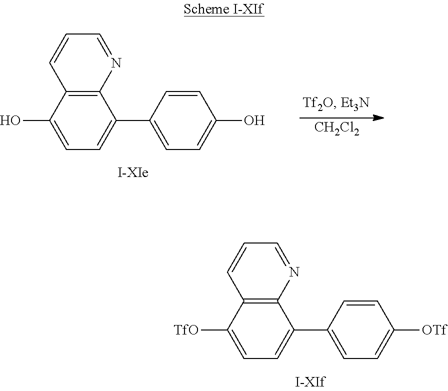

### General Procedure I-CX

To a solution of compound I-XIe (800 mg, 3.36 mmoL) and TEA (2.26 g, 8.07 mmol) in 20 mL of DCM was added Tf₂O (2.26 g, 8.07 mmol) dropwise at −20° C. The reaction mixture was stirred for 10 min. at −20° C., then 30 min. at ambient temperature. After quenched with 30 mL ice-water (5 mL), the mixture extracted with DCM (20 mL), washed with brine (10 mL), dried over Na₂SO₄ and concentrate under reduced pressure. The residue was purified by column chromatography (eluting with PE:EA=5:1) to give compound I-XIf (0.7 g, yield: 42%). MS (ESI) m/z (M+H)⁺ 502.

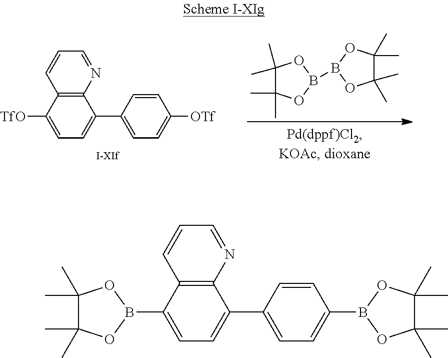

### General Procedure I-CY

A mixture of compound I-XIf (700 mg, 1.4 mmol), bis(pinacolato)diboron (851.7 mg, 3.35 mmol) and KOAc (274.4 mg, 2.8 mmol) and Pd(dppf)₂O₂ (70 mg) in 15 mL of dioxane was stirred at reflux overnight. Then it was concentrated and the residue was diluted with brine (10 mL), extracted with DCM (50 mL×3). The combined organic layers were dried over Na₂SO₄ and concentrate under reduced pressure. The residue was purified by column chromatography (eluting with PE:EA=10:1) to give compound I-XIg (250 mg, yield: 45.6%).

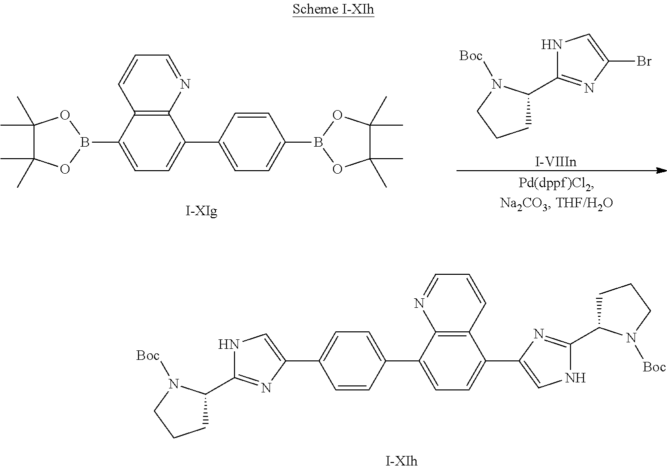

### General Procedure I-CZ

A mixture of compound I-XIg (100 mg, 0.22 mmol), compound I-VIIIn (164.4 mg, 0.52 mmol), Na₂CO₃ (93.28 mg, 0.88 mmol) and Pd(dppf)Cl₂ (16.0 mg, 0.022 mmol) in THF/H₂O (10 mL/1 mL) was stirred at reflux overnight. After concentrated under reduced pressure, the residue was diluted water, extracted with EtOAc. The organic layer was separated, dried over Na₂SO₄ and concentrated under reduced pressure. The residue was purified by Prep. HPLC to give compound I-XIh (95 mg, yield: 54.7%). MS (ESI) m/z (M+H)⁺ 676.

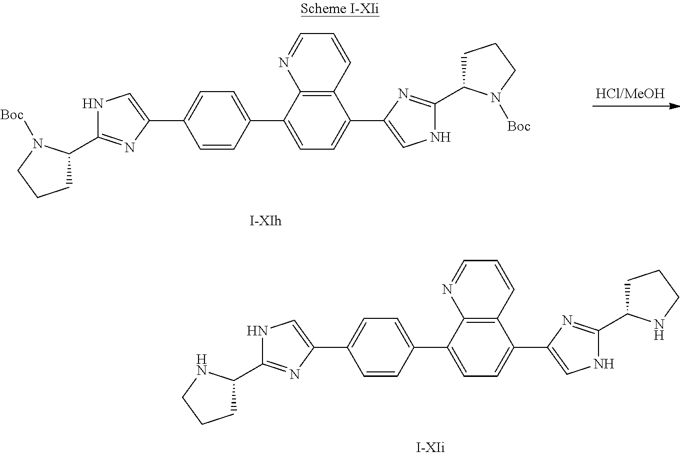

### General Procedure I-DA

A mixture of compound I-XIh (120 mg, 0.17 mmol) in 6 mL of 4M HCl/MeOH was stirred at room temperature for 1 h. Then the mixture was concentrated under reduced pressure to afford compound I-XIi, which was used for next step without further purification.

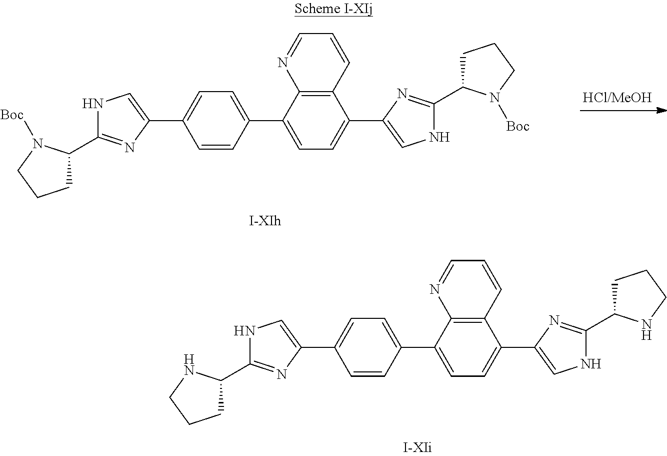

### General Procedure I-DA

A mixture of compound I-XIh (120 mg, 0.17 mmol) in 6 mL of 4M HCl/MeOH was stirred at room temperature for 1 h. Then the mixture was concentrated under reduced pressure to afford compound I-XIi, which was used for next step without further purification.

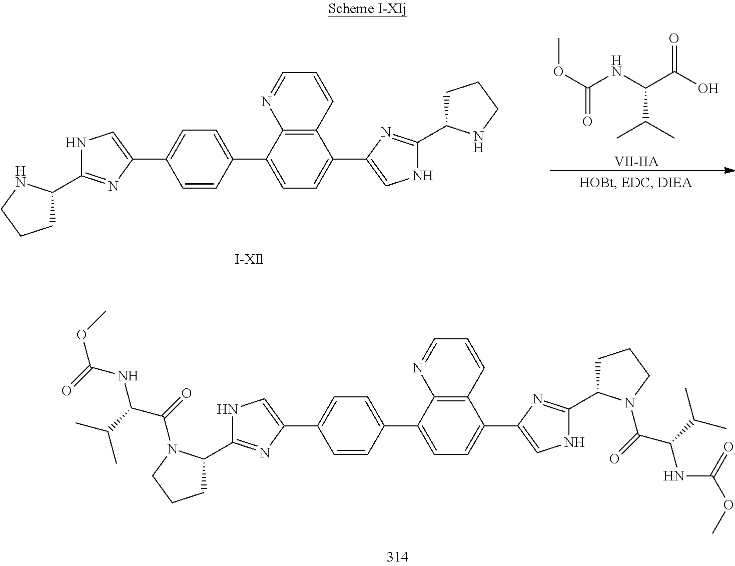

### General Procedure I-DA

Compound I-XIi (87.3 mg, 0.504 mmoL) was dissolved in 5 mL of CH₃CN, then HOBt (68.04 mg, 0.504 mmol) was added into the above solution, the mixture was stirred for about 10 minutes. Then compound VII-IIA (100 mg, 0.21 mmol), EDC (97 mg, 0.504 mmol) and DIEA (65 mg, 0.504 mmol) was added into the above reaction mixture. The reaction mixture was stirred at r.t. for 10 h. After diluted with water (5 mL) the mixture was extracted with EtOAc (20 mL). The organic layer was separated, dried with anhydrous MgSO₄, and concentrated under reduced the residue was purified by Prep. HPLC to afford compound 314 (18 mg, yield: 11%). ¹H NMR (300 MHz, CDCl₃): δ8.86 (s, 1H), 7.63 (m, 6H), 7.33 (m, 1H), 7.15 (s, 1H), 5.37 (m, 2H), 5.26 (m, 1H), 5.22 (m, 1H), 4.28 (m, 2H), 3.81 (m, 2H), 3.75 (m, 8H), 2.96 (s, 2H), 2.30 (m, 2H), 2.20 (m, 2H), 2.15 (m, 2H), 1.93 (m, 2H), 0.83 (m, 12H). MS (ESI) m/z (M+H)⁺ 79.

### Example I-XII

### Preparation of Compound 315

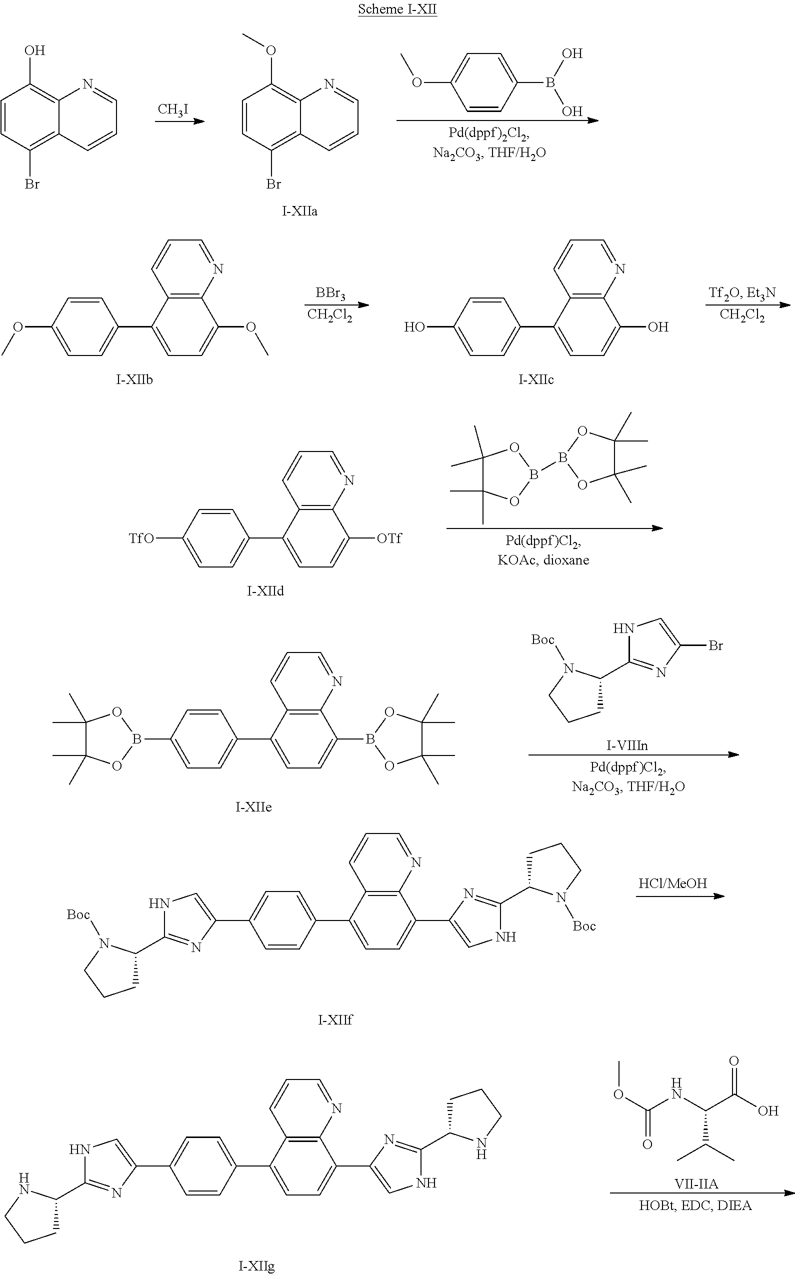

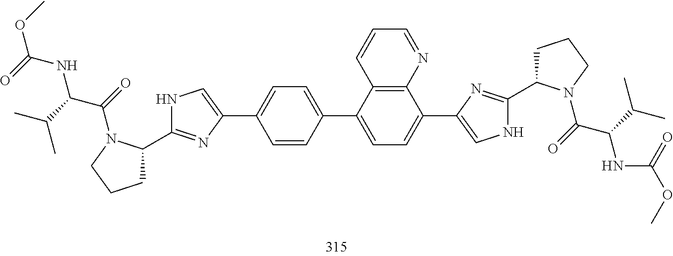

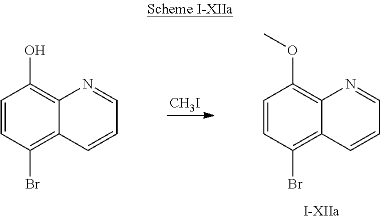

### General Procedure I-DB

CH₃I (5.68 g, 0.04 mol) was added to the mixture of 5-bromoquinolin-8-ol (8 g, 0.036 mol), potassium carbonate (5.68 g, 0.04 mol) in 50 mL of DMF. The reaction mixture was stirred for 5 h at r.t, then water was added, and the precipitate was collected by filtration to afford compound I-XIIa (5.5 g, 64%). MS (ESI) m/z (M+H)⁺ 238.

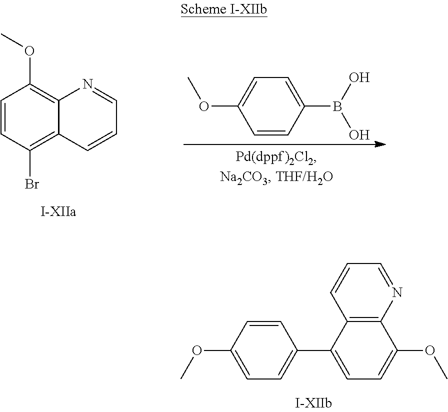

### General Procedure I-DC

A mixture of compound I-XIIa (3 g, 13 mmol), 4-methoxy-phenyl boronic acid (1.92 g, 13 mmol), and Pd(dppf)₂Cl₂, (0.475 g, 0.65 mmol) and Na₂CO₃ (2.75 g, 26 mmol) in THF/H₂O (36 mL/4 mL) was stirred at 80° C. overnight. After concentrated under reduced pressure and the residue was diluted with water, and extracted with EtOAc. The organic layer was separated, dried over Na₂SO₄ and concentrated under reduced pressure. The residue was purified by column chromatography (eluting with PE:EA=6:1) to afford compound I-XIIb (2.6 g, yield: 75%). MS (ESI) m/z (M+H)⁺ 266.

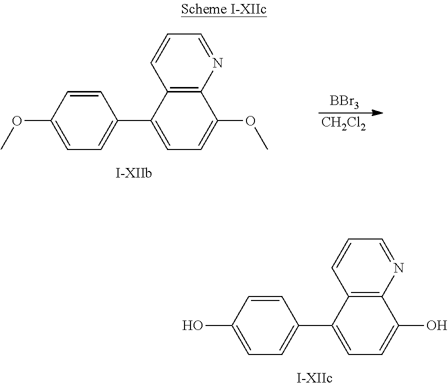

### General Procedure I-DD

BBr₃ (1.8 g, 7.16 mmoL) was added dropwise to the mixture of compound I-XIIb (900 mg, 3.58 mmoL) in 10 mL of anhydrous CH₂Cl₂ at −78° C. After addition, the reaction mixture was warmed to r.t. and stirred for 5 h. Then water (10 mL) was added, and extracted with EtOAc (100 mL×3), the organic layers was separated, dried, and concentrated under reduced pressure. The residue was purified by column chromatography (DCM/MeOH=8/1) to give compound I-XIIc (0.68 g, yield: 76%). MS (ESI) m/z (M+H)⁺ 238.

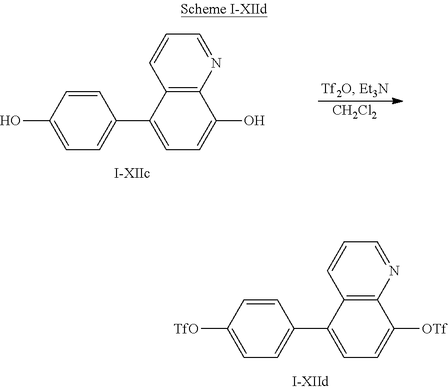

### General Procedure I-DE

To a solution of compound I-XIIc (800 mg, 3.36 mmoL) and TEA (2.26 g, 8.07 mmol) in 20 mL of DCM was added Tf₂O (2.26 g, 8.07 mmol) dropwise at −20° C. The reaction mixture was stirred for 10 min. at −20° C., then 30 min. at ambient temperature. After quenched with 30 mL ice-water (5 mL), the mixture extracted with DCM (20 mL), washed with brine (10 mL), dried over Na₂SO₄ and concentrate under reduced pressure. The residue was purified by column chromatography (eluting with PE:EA=5:1) to give compound I-XIId (0.7 g, yield: 42%). MS (ESI) m/z (M+H)⁺ 502).

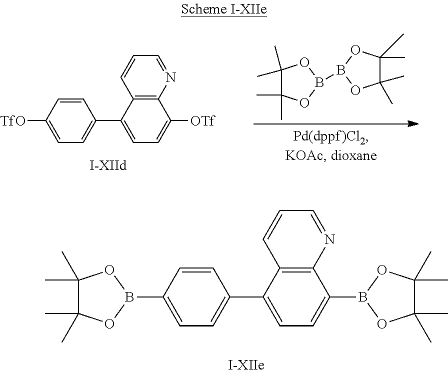

### General Procedure I-DF

A mixture of compound I-XIId (1 g, 1.99 mmol), bis(pinacolato)diboron (2 g, 7.87 mmol) and KOAc (782 mg, 7.97 mmol) and Pd(dppf)₂Cl₂ (146 mg) in 15 mL of dioxane was stirred at reflux overnight. Then it was concentrated and the residue was diluted with brine (10 mL), extracted with DCM (50 mL×3). The combined organic layers were dried over Na₂SO₄ and concentrate under reduced pressure. The residue was purified by column chromatography (eluting with PE:EA=10:1) to give compound I-XIIe (1.2 g, yield: 92%).

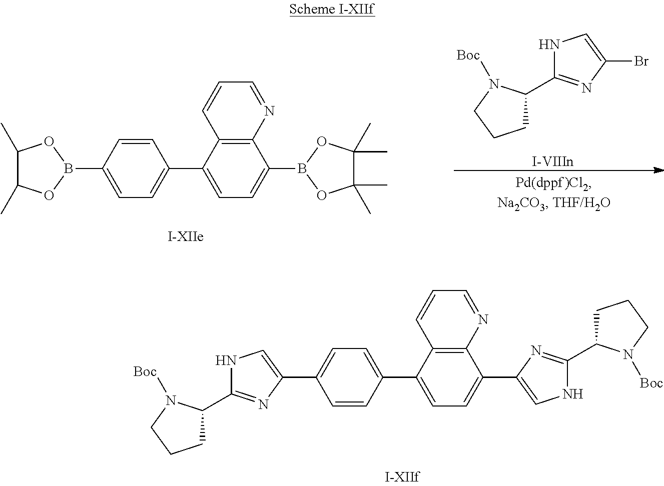

### General Procedure I-DG

A mixture of compound I-XIIe (500 mg, 1.09 mmol), compound I-VIIIn (665 mg, 2.10 mmol), Na₂CO₃ (463 mg, 4.37 mmol) and Pd(dppf)₂Cl₂ (80 mg, 0.11 mmol) in THF/H₂O (10 mL/1 mL) was stirred at reflux overnight. After concentration under reduced pressure, the remaining residue was diluted water, extracted with EtOAc. The organic layer was separated, dried over Na₂SO₄ and concentrated under reduced pressure. The residue was purified by Prep. HPLC to give compound I-XIIf (30 mg, yield: 6.5%). MS (ESI) m/z (M+H)⁺ 676.

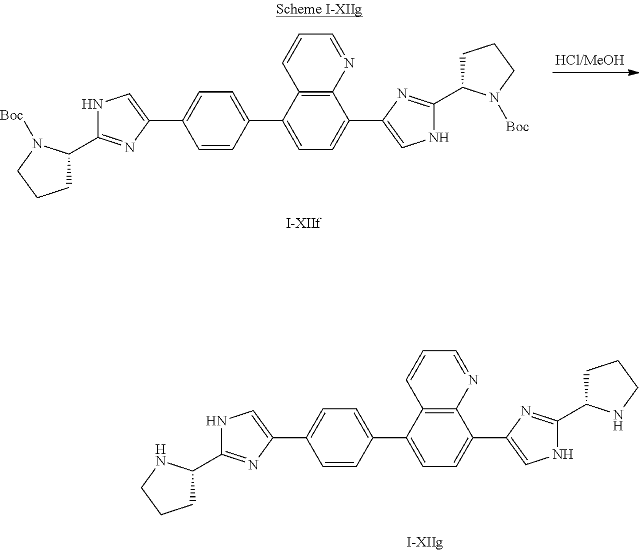

### General Procedure I-DH

The mixture of compound (30 mg, 0.038 mmol) in 6 mL of 4M HCl/MeOH was stirred at room temperature for 1 h. Then the mixture was concentrate under reduced pressure to give compound I-XIIg, which was used for next step without further purification.

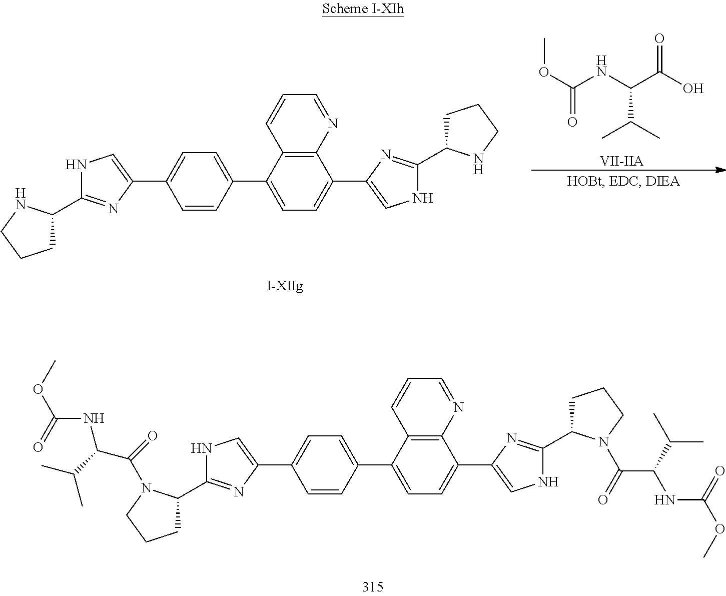

### General Procedure I-DI

Compound I-XIIg (23 mg, 0.048 mmoL) was dissolved in 5 mL of CH₃CN, then HOBt (17 mg, 0.1151 mmol) was added into the above solution, the mixture was stirred for about 10 min. Then compound VII-IIA (17 mg, 0.096 mmol), EDC (24 mg, 0.1151 mmol) and DIEA (15 mg, 0.1151 mmol) was added into the above reaction mixture. The reaction mixture was stirred at r.t. for 10 h. After diluted with water (5 mL) the mixture was extracted with EtOAc (20 mL). The organic layer was separated, dried with anhydrous MgSO₄, and concentrated under reduced the residue was purified by Prep. HPLC to afford compound 315 (8.3 mg, yield: 29.64%). ¹H NMR (300 MHz, CDCl₃): δ9.01 (d, J=2.8 Hz, 1H), 8.35 (d, J=8.4 Hz, 1H), 8.22 (d, J=7.6 Hz, 1H), 8.14 (s, 1H), 7.82 (d, J=8.4 Hz, 1H), 7.76 (s, 1H), 7.63 (d, J=8 Hz, 1H), 7.56 (d, J=7.6 Hz, 2H), 7.13 (m, 1H), 7.06 (m, 1H), 5.38 (m, 2H), 5.15 (m, 2H), 4.14 (m, 2H), 4.01 (m, 2H), 3.96 (m, 2H), 3.61 (m, 3H), 3.55 (m, 3H), 2.47 (m, 3H), 2.05 (m, 3H), 0.83 (m, 12H). MS (ESI) m/z (M+H)⁺ 790.

### Example I-XIII

### Preparation of Compound 316

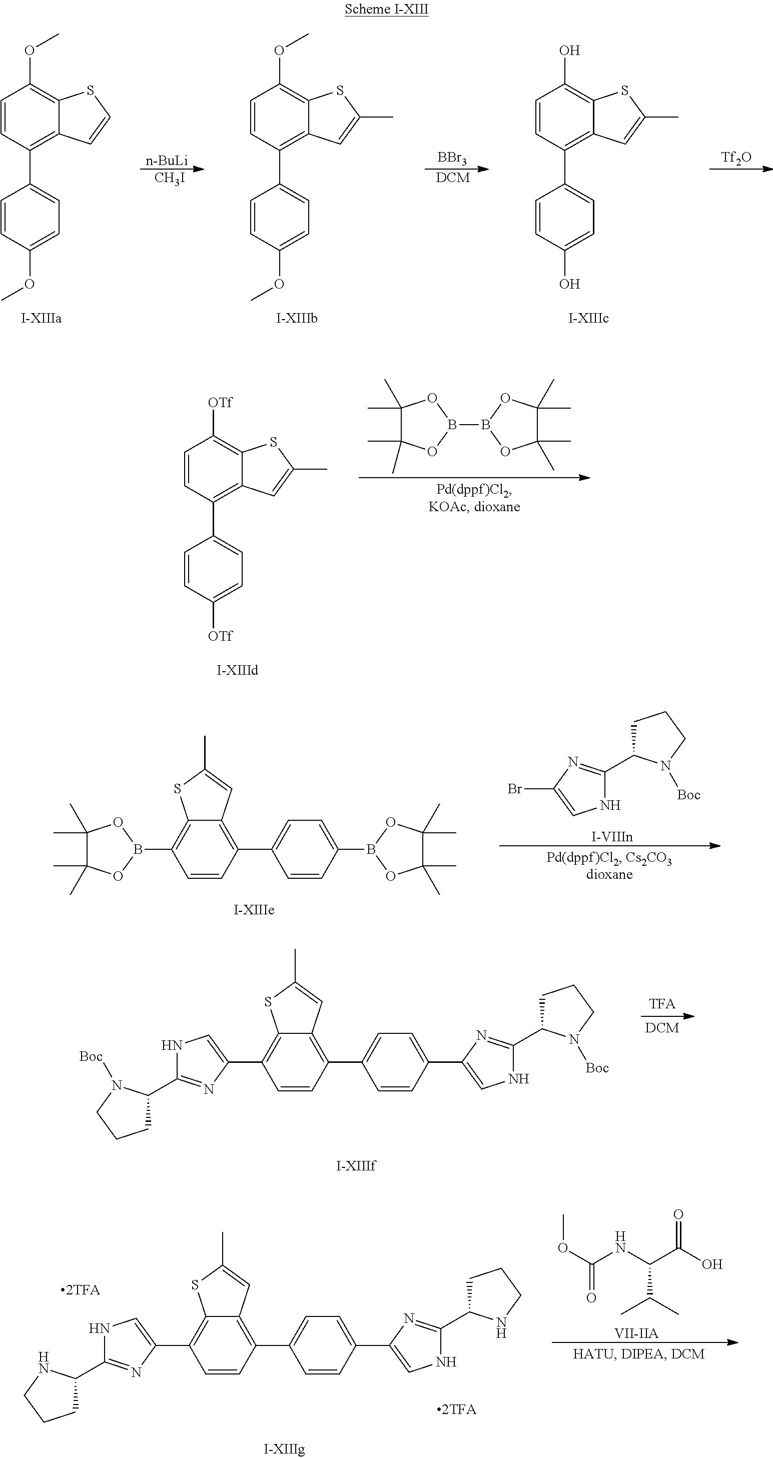

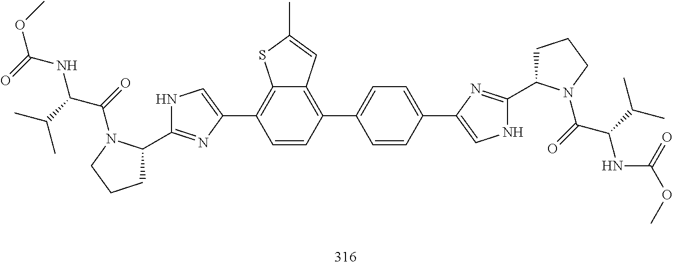

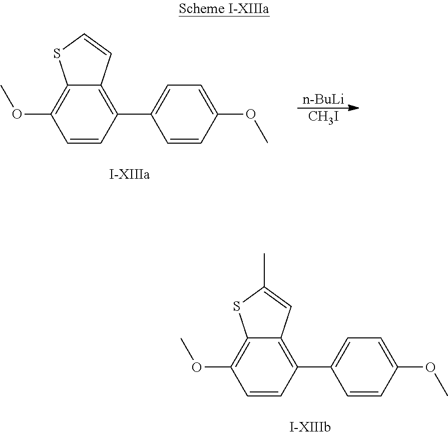

### General Procedure I-DJ

To a stirred solution of compound I-XIIIa (5.8 g, 2.15 mmol) in THF (100 mL) was added n-BuLi (2.5 M solution in hexanes, 6.2 mL, 15.7 mmol) at −70° C. and the mixture was stirred for 30 min., then CH₃I (6.1 g, 4.3 mmol) was added dropwise, and the reaction mixture was stirred for another 1 h. The reaction was quenched with saturated aq. NH₄Cl and extracted with EtOAc (20 mL×3), the combined organic layers were washed with brine, dried over MgSO₄ and concentrated. The residue was purified by column chromatography to afford compound I-XIIIb (1.6 g, yield 26%). ¹H NMR (400 MHz, CDCl₃) δ 7.36 (d, J=8.7 Hz, 2H), 7.14 (d, J=8.0 Hz, 1H), 6.99 (s, 1H), 6.92 (d, J=8.6 Hz, 2 H), 6.69 (d, J=8.0 Hz, 1H), 3.93 (s, 3H), 3.79 (s, 3H), 2.49 (s, 3H).

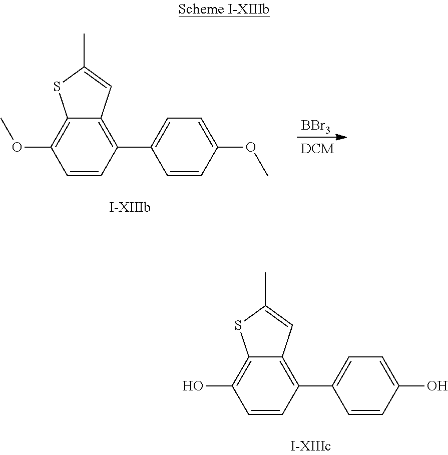

### General Procedure I-DK

BBr₃ (5.5 g, 22 mmol) was added to a stirred solution of compound I-XIIIb (1.2 g, 4.4 mmol) in DCM at 0° C. The reaction mixture was stirred for 30 min. The mixture was poured into ice-water, and extracted with DCM. The combined organic layers were dried over MgSO₄ and concentrated to afford compound I-XIIIc (0.5 g, yield 44%).

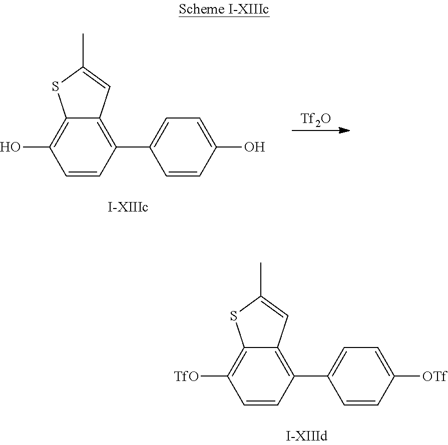

### General Procedure I-DL

Tf₂O (1.65 g, 5.86 mmol) was added to a stirred solution of compound I-XIIIc (500 mg, 1.95 mmol) and DIEA (760 mg, 5.86 mmol) in DCM (10 mL) at 0° C. The reaction mixture was stirred for 30 min, then poured into ice-water, and extracted with DCM. The combined organic layers were dried over MgSO₄ and concentrated in vacuo to afford compound I-XIIId (0.65 g, yield 65%).

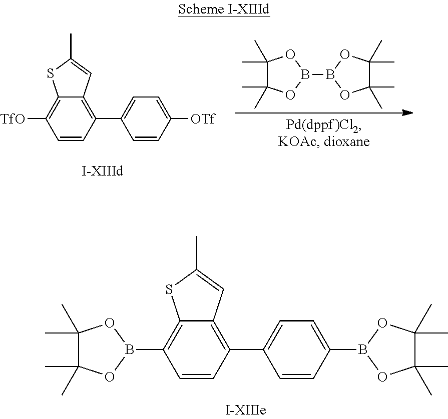

### General Procedure I-DM

To a stirred mixture of compound I-XIIId (650 mg, 1.25 mmol), bis(pinacolato)diboron (950 mg, 3.75 mmol) and KOAc (370 mg, 3.75 mmol) in 1,4-dioxane (10 ml) was added Pd(dppf)Cl₂ (50 mg) under N₂ protection. The mixture was stirred at 80° C. for 3 hrs. Then the mixture was diluted with EtOAc, washed with water and brine, the organic layers was dried over MgSO₄, filtered and concentrated. The residue was purified by prep-TLC to afford compound I-XIIIe (400 mg, yield 67%). MS (ESI) m/z (M+H)⁺ 477.3.

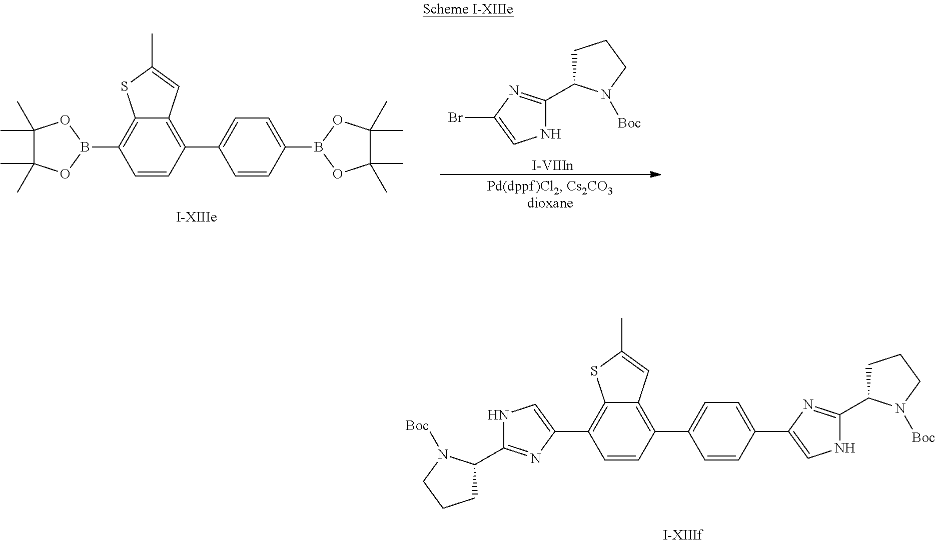

### General Procedure I-DN

To a stirred mixture of compound I-XIIIe (400 mg, 0.84 mmol), compound I-VIIIn (794 mg, 2.52 mmol), and Cs₂CO₃ (890 mg, 2.52 mmol) in 1,4-dioxane (5 mL) and H₂O (1 mL) was added Pd(dppf)Cl₂ (50 mg) under N₂ protection. The mixture was stirred at 80° C. for 3 hrs. Then the mixture was diluted with EtOAc, washed with water and brine, the organic layers was dried over MgSO₄, filtered and concentrated. The residue was purified by prep-TLC to afford compound I-XIIIf (200 mg, yield 34%). MS (ESI) m/z (M+H)⁺ 695.3.

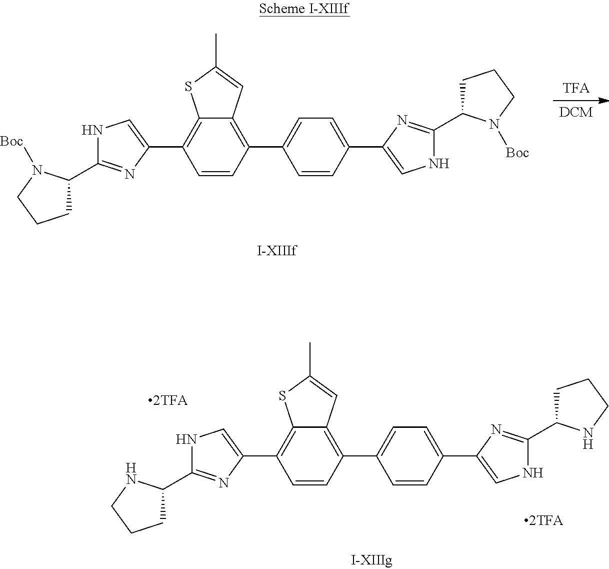

### General Procedure I-DO

To a solution of compound I-XIIIf (230 mg, 0.33 mmol) in DCM was added TFA (3 mL). The reaction mixture was stirred at r.t. for 1 h. The solvent was concentrated under reduced pressure, neutralized with aq.NaHCO₃, and extracted with DCM. The combined organic layer was dried over MgSO₄, filtered and concentrated to give compound I-XIIIg (100 mg, yield 61%), which was used directly in the next reaction without further purification. MS (ESI) m/z (M+H)⁺ 495.3

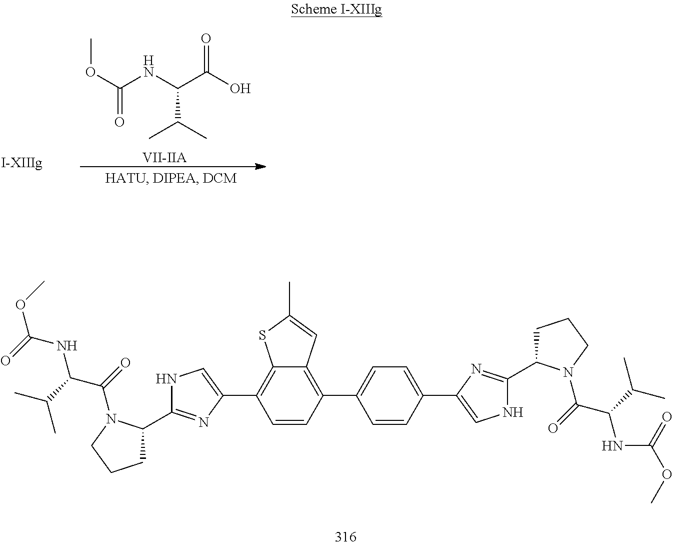

### General Procedure I-DP

To a stirred mixture of compound I-XIIIg (190 mg, 0.2 mmol), HATU (266 mg, 0.7 mmol) and DIEA (210 mg, 1.6 mmol) in DCM was added compound VII-IIA (106 mg, 0.606 mmol). The reaction mixture was stirred at r.t. for 1 h. Then the mixture was diluted with DCM, washed with water and brine, the organic layer was separated, dried, filtered and concentrated under reduced pressure. The residue was purified by prep-HPLC to afford compound 316 (55.3 mg, yield 34%). ¹H NMR (400 MHz, CDCl₃) δ 7.89-7.62 (m, 2H), 7.59-7.45 (m, 3H), 7.45-7.38 (m, 1H), 7.36-7.25 (m, 2H), 7.08 (s, 1H), 5.39 (d, 2H), 5.30-5.15 (m, 2H), 4.27 (t, 2H), 3.83-3.74 (m, 2H), 3.74-3.53 (m, 8H), 3.23-2.84 (m, 2H), 2.51 (s, 3H), 2.46-2.25 (m, 2H), 2.25-2.00 (m, 4H), 1.96-1.85 (m, 2H), 0.83 (s, 6H), 0.81 (s, 6H). MS (ESI) m/z (M+H)⁺ 809.4.

### Example I-XIV

### Preparation of Compound 317

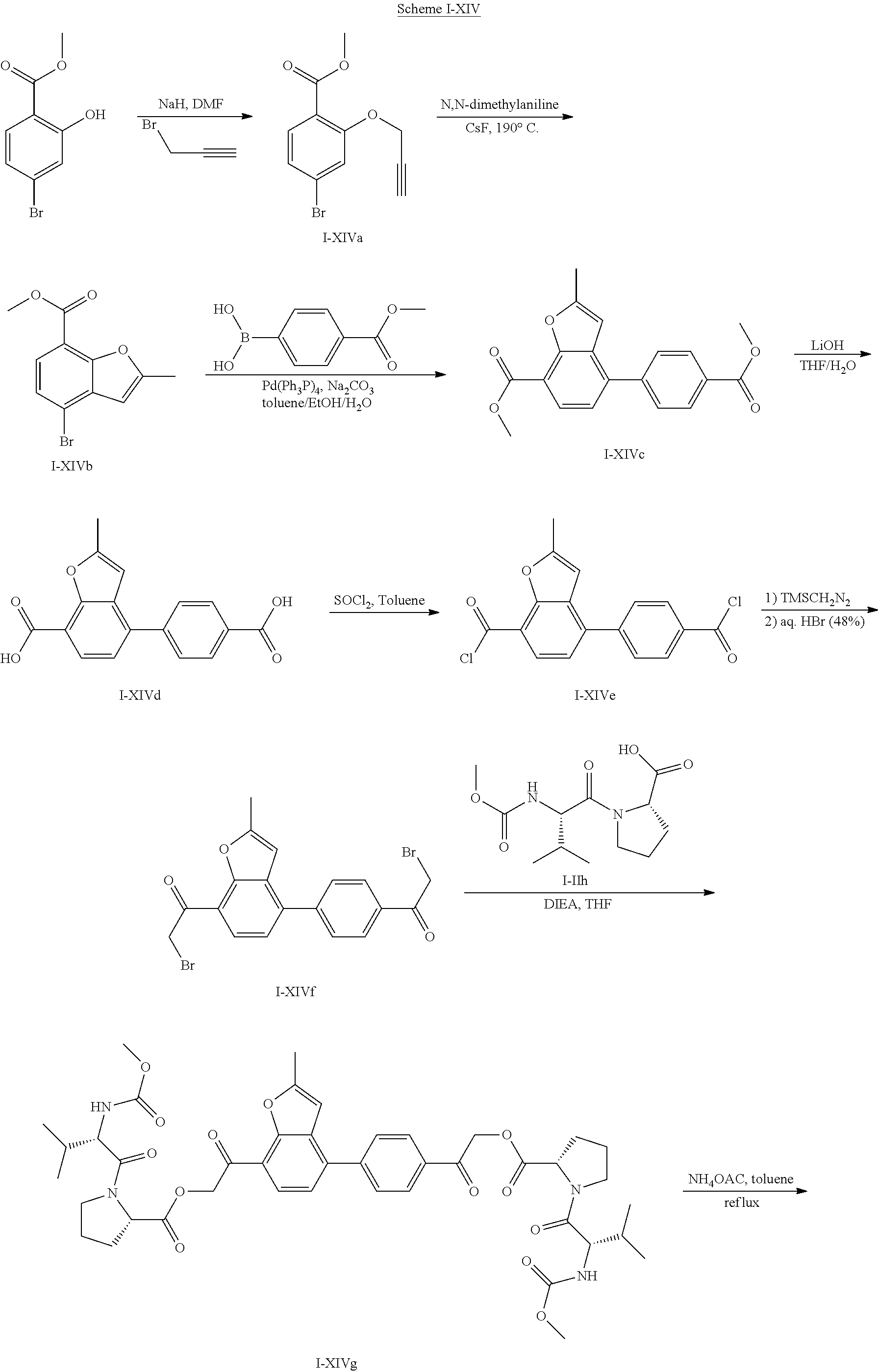

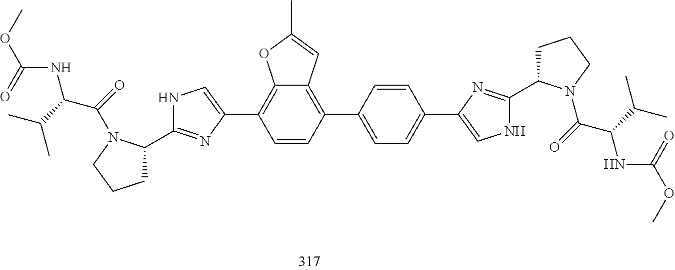

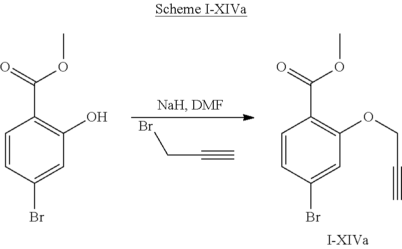

### General Procedure I-DQ

To a solution of methyl 4-bromo-2-hydroxybenzoate (4.6 g, 20.0 mmol) in DMF (50 mL) was added sodium hydride (60% dispersion in mineral oil, 1.2 g, 30.0 mmol) at 0° C. and the mixture was stirred for 30 minutes at the same temperature. After that, 3-bromo-propyne (3.5 g, 30.0 mmol) in DMF (5 ml) was added dropwise at 0° C. and the mixture was stirred at r.t. for 6 hours. The solvent was removed and the residue was dissolved in ethyl acetate, washed with water, brine and dried over sodium sulfate. The solvent was removed under reduced pressure to afford compound I-XIVa (4.8 g, yield 91%) as yellow oil. ¹H NMR (400 MHz, CDCl₃) δ 7.68 (d, 1H), 7.26 (d, 1H), 7.18 (d, 1H), 4.76 (s, 1H), 3.85 (s, 3H), 2.55 (s, 1H).

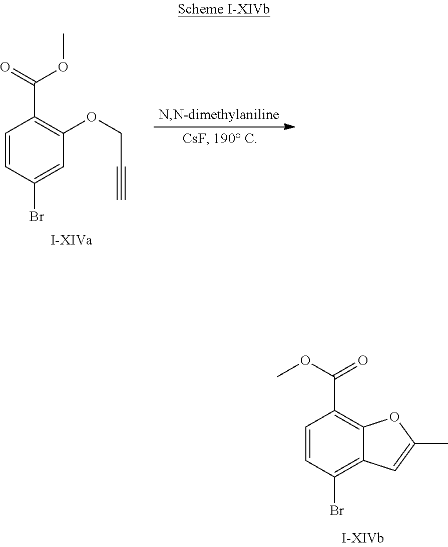

### General Procedure I-DR

Compound I-XIVa (2.7 g, 10 mmol) and CsF (1.5 g, 10 mmol) were charged into a 50 mL flask and the reaction flask was flushed with N₂ (nitrogen gas), N,N-dimethylaniline (10 mL) was added and the reaction mixture was heated at 190° C. for 4 hrs. After cooling to room temperature, water was added and the reaction mixture was extracted with EtOAc (50 mL×3). The combined organic layers were washed with aq. HCl (1 N) and brine and dried over Na₂SO₄. Purification of the concentrated crude product by column chromatography (PE:EtOAc=1:4) afforded compound I-XIVb (1.1 g, yield 39%) as yellow solid. ¹H NMR (400 MHz, CDCl₃) δ 7.72 (d, 1H), 7.38 (d, 1H), 6.48 (s, 1H), 3.98 (s, 3H), 2.53 (s, 3H).

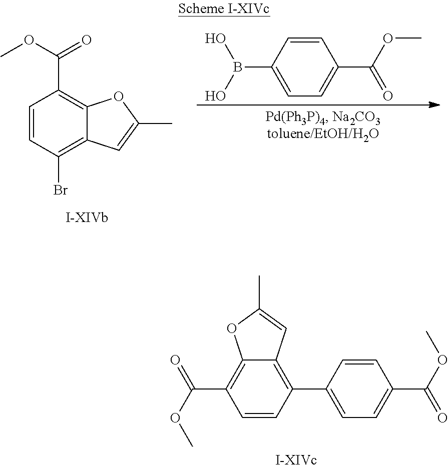

### General Procedure I-DS

To a solution of compound I-XIVb (540 mg, 2.0 mmol) in toluene (15 mL) were added EtOH (2 mL), aq. Na₂CO₃ (2.0 M, 1.5 mL, 3.0 mmol), 4-(methoxycarbonyl)phenylboronic acid (450 mg, 2.5 mmol). The mixture was purged with N₂ (nitrogen gas), and then, Pd(Ph₃P)₄ (60 mg, 0.05 mmol) was added and the mixture was stirred at 90° C. under nitrogen atmosphere for 12 hours. After the reaction completed, the solvent was removed under reduced pressure and the residue was dissolved in ethyl acetate. The organic layer was washed with water, brine and dried over sodium sulfate. The solvent was removed and the residue was purified by column chromatography (PE:EtOAc=1:4) to yield compound I-XIVc (520 mg, yield 80%) as yellow solid. ¹H NMR (400 MHz, CDCl₃) δ 8.16 (d, 2H), 7.92 (d, 1H), 7.68 (d, 2H), 7.34 (d, 1H), 6.58 (s, 1H), 4.02 (s, 3H), 3.96 (s, 3H), 2.56 (s, 3H).

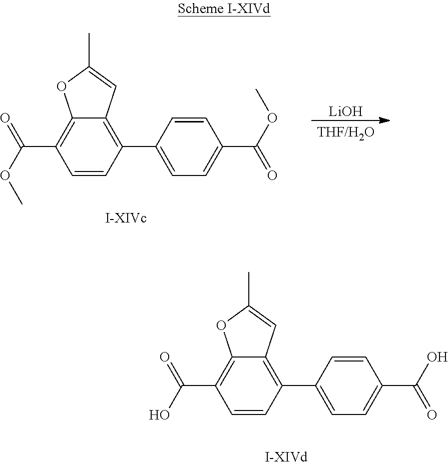

### General Procedure I-DS

To a solution of compound I-XIVc (648 mg, 2.0 mmol) in THF (15.0 mL) was added aq. LiOH (2.0 M, 10.0 mL, 20.0 mmol) and the mixture was stirred at r.t. for 24 hrs. After the reaction completed, the solvent was removed under reduced pressure and water was added, the pH value of the mixture was adjusted to 2 with aq. HCl (1 N) and the solid was collected by filtration. The wet solid was dried to afford compound I-XIVd (480 mg, yield 80%) as white solid.

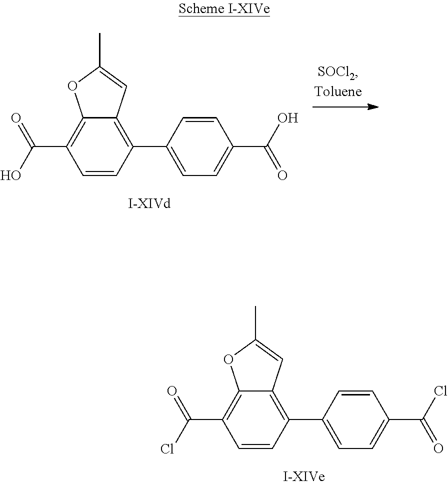

### General Procedure I-DT

A mixture of compound I-XIVd (300 mg, 1.0 mmol) and SOCl₂ (5.0 mL) was heated to reflux for 3 hrs. Subsequently, the excess SOCl₂ was removed under reduced pressure to afford compound I-XIVe (331 mg, yield 100%) as yellow solid, compound I-XIVe was used directly in next step.

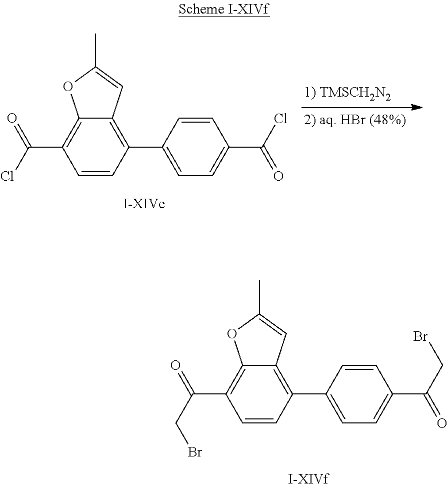

### General Procedure I-DU

To a solution of compound I-XIVe (331 mg, 1.0 mmol) in dry DCM (15.0 mL) was added trimethylsilyl diazomethane (TMSCH₂N₂, 2.0 M solution in hexanes, 3.0 ml, 6.0 mmol) at −10° C. and the mixture was stirred at 0° C. for 1 hr, and then, the mixture was cooled to −10° C. again, aq. HBr (40% solution, 3.0 mL) was added dropwise at the same temperature. The temperature of the reaction mixture was slowly warmed to r.t. and stirred for 1 hr and extracted with DCM and washed with water, saturated aq. NaHCO₃, and brine. The organic phase was dried over sodium sulfate and concentrated to give compound I-XIVf (310 mg, yield 70%) as yellow solid.

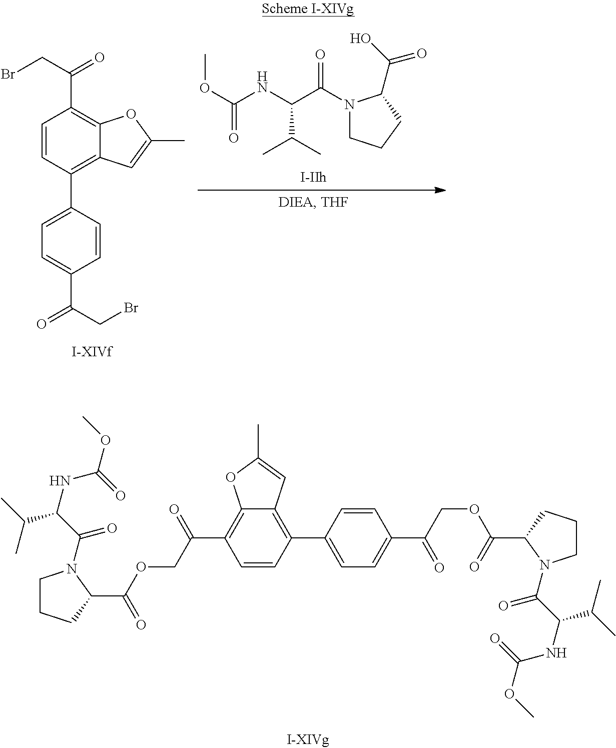

### General Procedure I-DV

To a solution of compound I-XIVf (230 mg, 0.50 mmol) in THF (10.0 mL) were added compound I-IIh (340 mg, 1.2 mmol) and DMA (800 mg, 6.0 mmol) and the mixture was stirred at r.t. for 12 hours. After the reaction completed, the solvent was removed under reduced pressure and the residue was dissolved in DCM, washed with aq. HCl (1.0 N), brine and dried over sodium sulfate. The solvent was removed and the residue was purified by column chromatography to afford compound I-XIVg (185 mg, yield 45%) as yellow gum. ¹H NMR (400 MHz, CDCl₃) δ 11.14 (s, 1H), 7.18 (d, 1H), 6.96 (d, 1H), 5.93 (br, 1H), 4.34 (q, 2H), 1.34 (t, 3H).

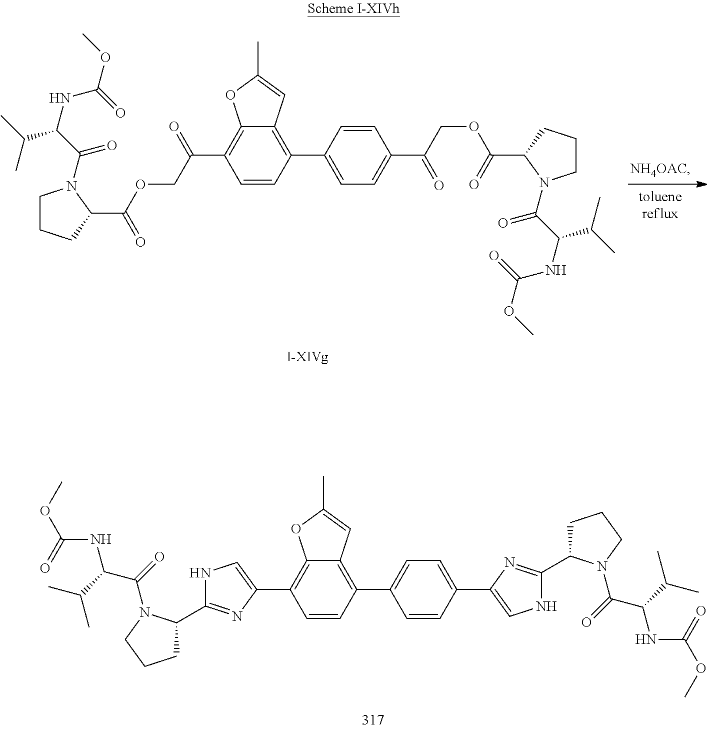

### General Procedure I-DW

To a solution of compound I-XIVg (125 mg, 0.15 mmol) in toluene (10.0 mL) was added ammonium acetate (1.54 g, 20.0 mmol) and the mixture was refluxed for 24 hrs. While the reaction completed, the mixture was cooled to r.t. and solvent was evaporated. The residue was diluted with DCM, the resulting solution was washed with water, brine and dried over sodium sulfate. The solvent was removed under reduced pressure and the residue was purified by prep-HPLC to provide compound 317 (15.0 mg, yield 15%) as yellow solid. ¹H NMR (400 MHz, CDCl₃) δ 7.85-7.72 (m, 2H), 7.59-7.52 (m, 3H), 7.31-7.28 (m, 3H), 6.61 (s, 1H), 5.72-5.30 (m, 4H), 4.37-4.30 (m, 2H), 3.85-3.67 (m, 10H), 3.08-3.02 (m, 2H), 2.54 (s, 3H), 2.42-1.98 (m, 10H), 1.35-1.31 (m, 3H), 0.97-0.91 (m, 13H). MS (ESI) m/z (M+H)⁺ 793.3.

### Example I-XV

### Preparation of Compounds 318 and 319

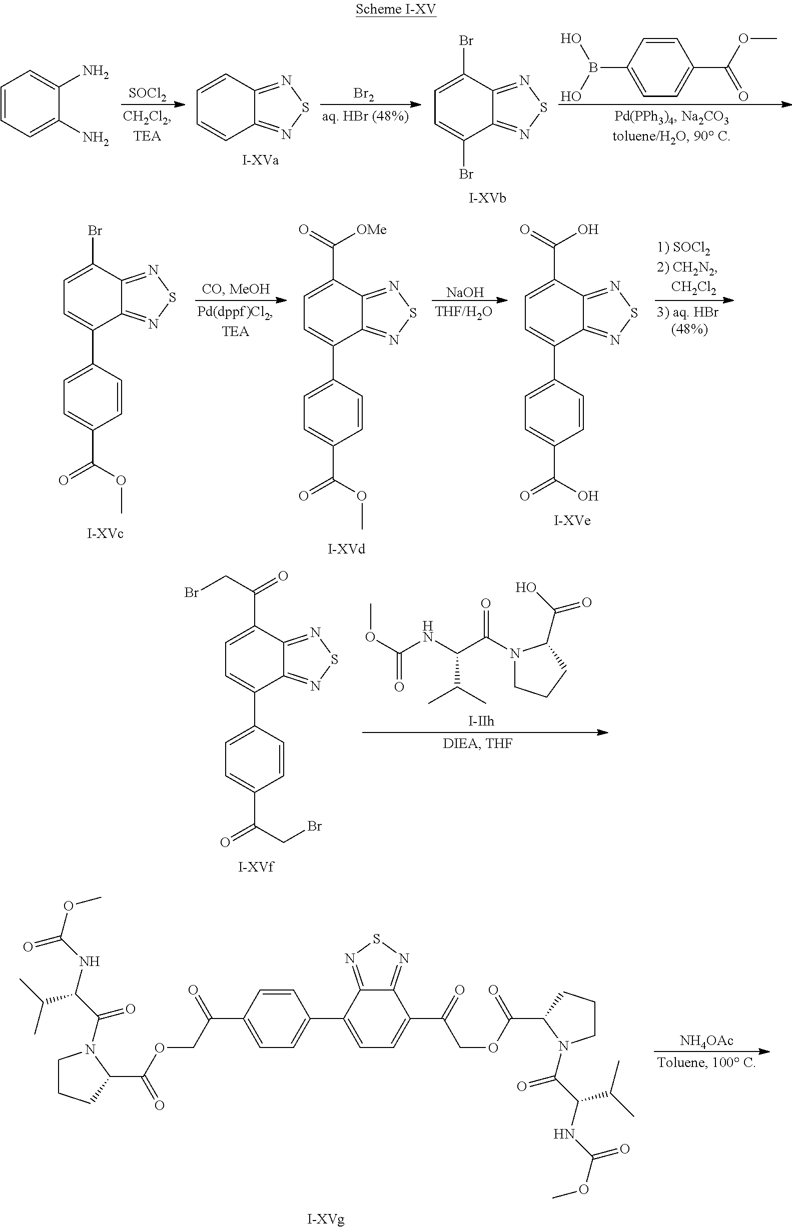

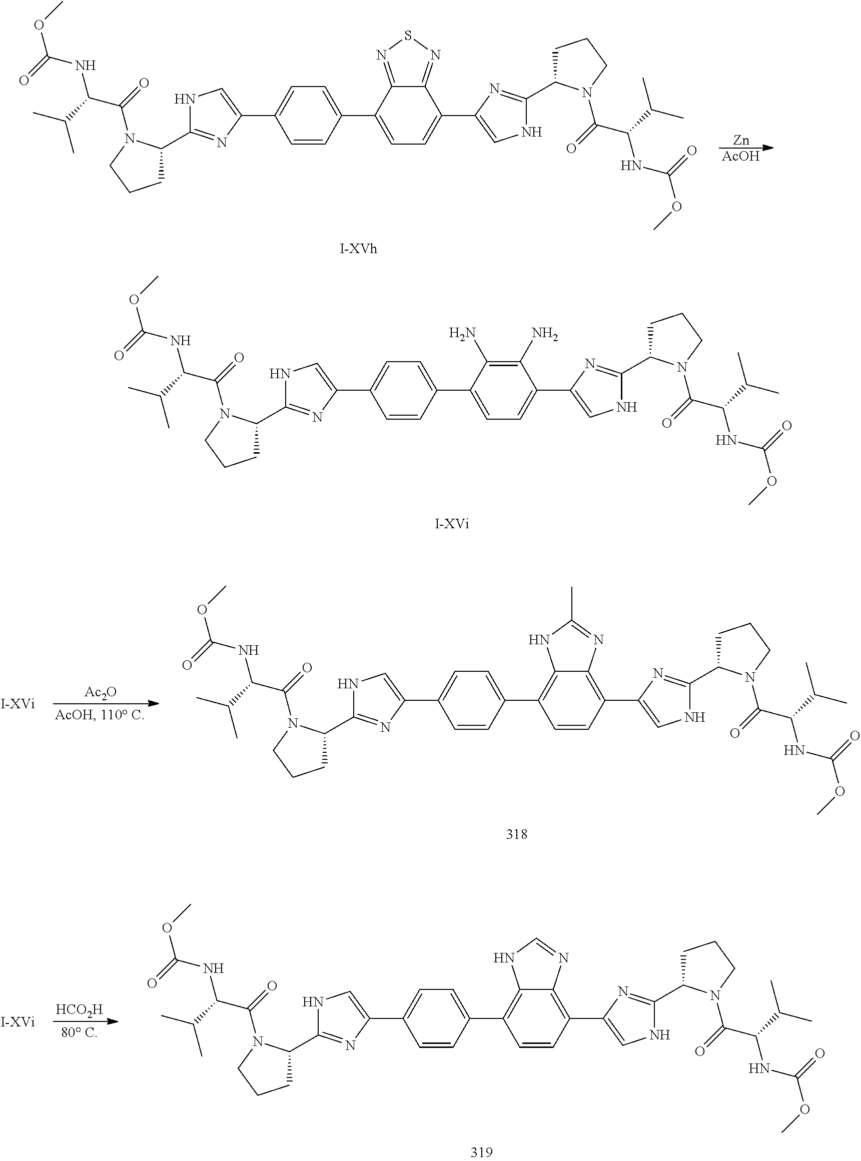

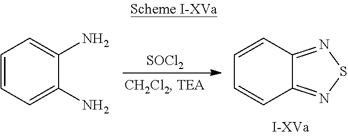

### General Procedure I-DX

A 1000 mL of flask were charged with 1,2-phenylenediamine (10.0 g, 92.5 mmol), CH₂Cl₂ (300 mL) and triethylamine (37.4 g, 370 mmol). The solution was stirred until 1,2-phenylenediamine dissolved. Thionyl chloride (22.04 g, 184.9 mmol) was added dropwise very slowly, and then the mixture was heated to reflux for 5 hrs. The solvent was removed under reduced pressure, and water (700 mL) was added. Conc. HCl was added to achieve a final pH=1. The mixture was extracted with CH₂Cl₂ (200 mL×3), the combined organic layers were washed with brine, dried over anhydrous Na₂SO₄, and concentrated in vacuo to afford compound I-XVa as a dark red solid (11.7 g, yield 93%).

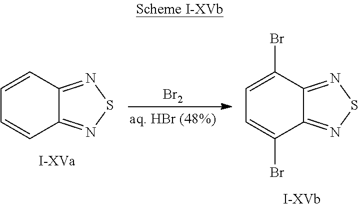

### General Procedure I-DY

A mixture of compound I-XVa (10.0 g, 73.4 mmol) in aq. HBr (48%, 150 mL) was heated to reflux with stirring, while a solution containing Br₂ (35.2 g, 220.3 mmol) in aq. HBr (48%, 100 mL) was added dropwise very slowly. Towards the end of the addition, the mixture became a suspension. To facilitate stirring, aq. HBr (48%, 50 mL) was added, and the reaction mixture was heated to reflux for 4 hours after completion of the Br₂ addition. The mixture was filtered while hot and filtrate was washed with water. The crude product was dried and recrystallized from MeOH to give compound I-XVb as a white needles (16.5 g, yield 77%).

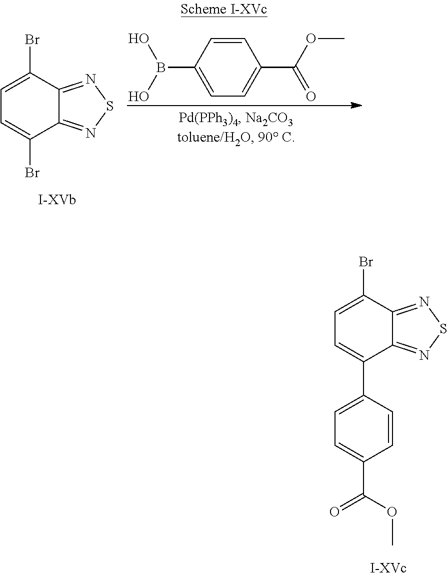

### General Procedure I-DZ

A mixture of compound I-XVb (5.0 g, 17.0 mmol), 4-(methoxycarbonyl)phenylboronic acid (5.0 g, 17.0 mmol), Pd(PPh₃)₄ (2.0 g, 1.7 mmol) and Na₂CO₃ (1.8 g, 17.0 mmol) was dissolved in toluene (80 mL) and H₂O (16 mL). The mixture was purged with N₂ (nitrogen gas) and heated at 90° C. for 12 hrs under N₂ (nitrogen gas) protection. After cooling, the mixture was poured into water and extracted with EtOAc. The organic layer was washed with brine and dried with anhydrous Na₂SO₄. After the solvent was evaporated, the residue was purified by column chromatograph on silica gel to give compound I-XVc as a pale yellow solid (2.0 g, yield 34%). ¹H NMR (400 MHz, CDCl₃) δ: 3.89 (s, 3H), 7.64 (d, 1H), 7.89 (t, 3H), 8.12 (d, 2H).

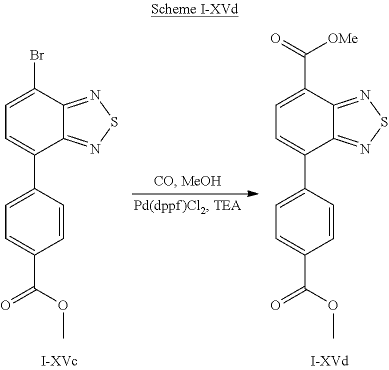

### General Procedure I-EA

An autoclave was charged with compound I-XVc (2.0 g, 5.73 mmol), triethylamine (1.17 g, 10.46 mmol), Pd(dppf)Cl₂ (0.48 g, 0.573 mmol) and MeOH (200 mL). The suspension was degassed under vacuum and purged with CO (carbon monoxide) three times, the reaction mixture was stirred at 120° C. for 16 hrs under CO (carbon monoxide) atmosphere with a pressure of 2 MPa. Then the suspension was filtered through a pad of celite and washed with MeOH. The combined filtrate was concentrated to dryness under reduced pressure. The residue was purified by column chromatograph on silica gel to give compound I-XVd as a yellow solid (1.1 g, yield 58%). ¹H NMR (400 MHz, CDCl₃) δ: 3.96 (s, 3H), 4.08 (s, 3H), 8.00-8.48 (m, 6H).

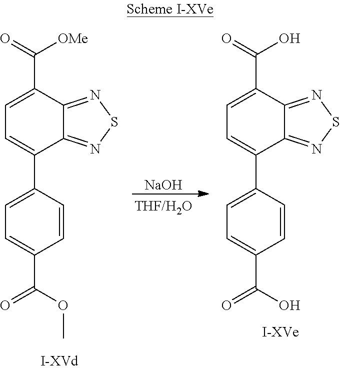

### General Procedure I-EB

To a solution of compound I-XVd (0.75 g, 2.28 mmol) in THF/H₂O (50 mL, 3 mL/1 mL) was added NaOH (0.274 g, 6.84 mmol). The reaction mixture was stirred at 40° C. overnight. The solvent was removed in vacuo and residual aqueous solution was partitioned with EtOAc (20 mL) then the organic phase was extracted with H₂O. The combined aqueous extract was acidified with to pH with 1N HCl. The aqueous phase was extracted with EtOAc. The combined organic extract was dried over anhydrous Na₂SO₄, and concentrated to give compound I-XVe as a white solid (0.61 g, yield 89%).

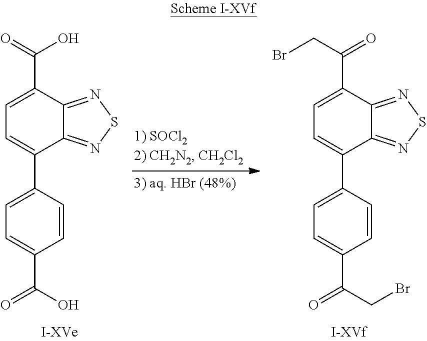

### General Procedure I-EC

A mixture of compound I-XVe (0.61 g, 2.03 mmol), SOCl₂ (8.8 mL, 121.8 mmol) (adding two drops of DMF) was refluxed for 2 hr. The excess of SOCl₂ was removed under reduced pressure. The residue was coevaporated with toluene (5 mL) for three times. The residue was dissolved in CH₂Cl₂ (5 mL) and the resulting solution was added dropwise to a solution of CH₂N₂ in ether (0.7 M, 30 mL, 21 mmol) at −10° C. The reaction mixture was stirred at 0° C. for 1 h. The reaction mixture was cooled to −10° C. again, to this solution was added dropwise aqueous HBr (48%, 2.4 mL, 20.3 mmol). The reaction mixture was stirred at the same temperature for 1 h, washed with saturated aqueous NaHCO₃ and brine. The organic phase was dried over anhydrous Na₂SO₄, and concentrated to give compound I-XVf as a brown solid (0.78 g, yield 85%). ¹H NMR (300 MHz, CDCl₃) δ: 4.50 (s, 2H), 5.16 (s, 2H), 8.11-8.16 (m, 4H), 7.91 (d, 1H), 8.51 (d, 1H).

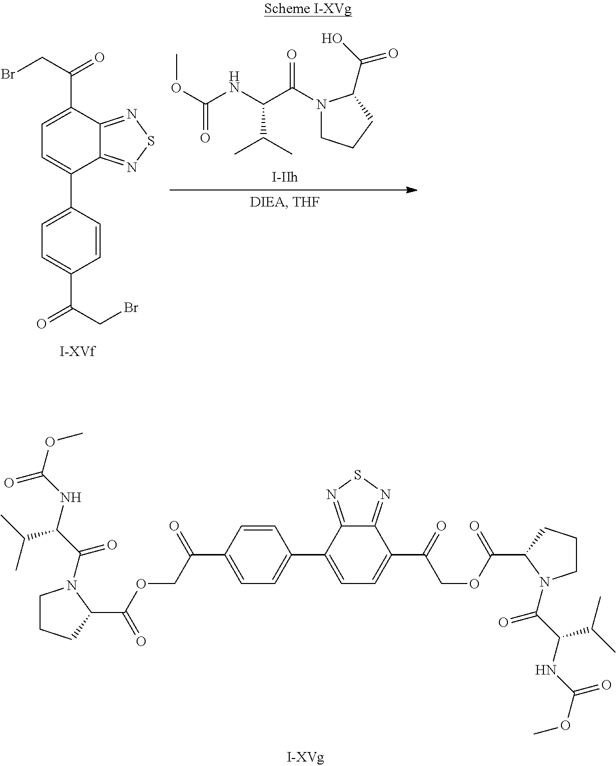

### General Procedure I-ED

Diisopropylethylamine (0.32 ml, 1.96 mmol) and compound I-XVf (384 mg, 1.78 mmol) were added to a suspension of compound I-IIh (270 mg, 0.59 mmol) in THF (10 mL). The resulting mixture was stirred at 40° C. overnight. After cooling to r.t., brine was added. The layers were separated, and the organic layer was dried over anhydrous Na₂SO₄, and concentrated. The residue was purified by column chromatograph on silica gel to afford compound I-XVg as a pale brown solid (190 mg, yield 38%).

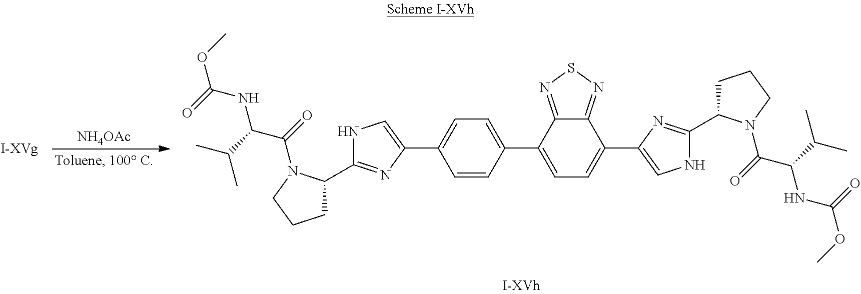

### General Procedure I-EE

To a solution of compound I-XVg (190 mg, 0.227 mmol) in toluene (15 mL) was treated with ammonium acetate (353 mg, 4.54 mmol), and reaction mixture was heated at 100° C. overnight. The solvent was removed under reduced pressure to dryness, the residue was purified by column chromatograph on silica gel to provide compound I-XVh as a red orange solid (140 mg, yield 77%).

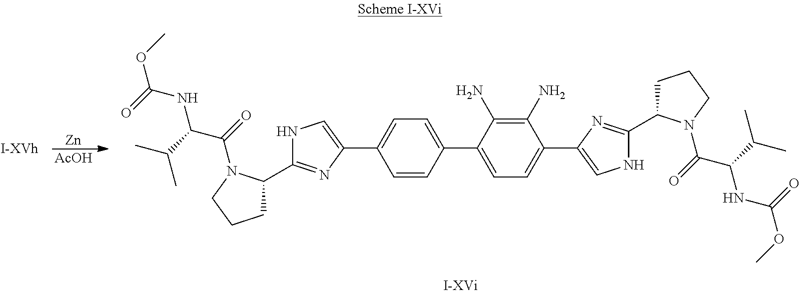

### General Procedure I-EF

To a suspension of compound I-XVh (110 mg, 0.138 mmol) in acetic acid (10 mL) and H₂O (2 mL) was added. Zn dust (181 mg, 2.76 mmol). The reaction mixture was stirred for 2 hrs at 70° C. After warm solution was poured into aq. NaOH (1 N), the aqueous layer was extracted with EtOAc. The combined organic layer was dried over anhydrous Na₂SO₄, and concentrated to give compound I-XVi as a yellow solid (100 mg, yield 93%), which was used directly without further purification.

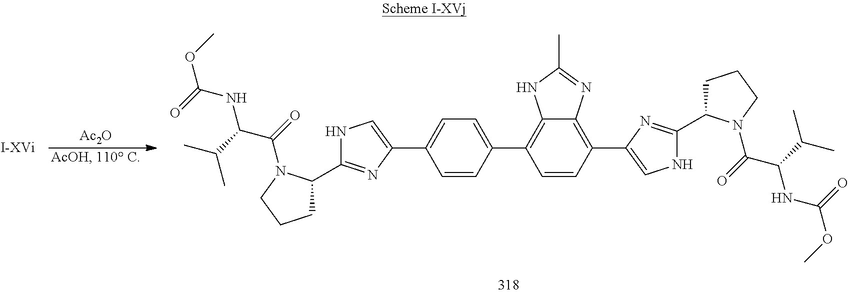

### General Procedure I-EG

A solution of compound I-XVi (50 mg, 0.065 mmol) and acetic anhydride (10 mg, 0.098 mmol) in acetic acid (5 mL) was heated at 100° C. for 2 hrs. Then the mixture was cooled to r.t. and diluted with water, neutralized with saturated aqueous NaHCO₃, extracted with EtOAc. The combined organic layers were washed with brine, dried over anhydrous Na₂SO₄, and concentrated in vacuo to dryness. The residue was purified by Prep-HPLC to yield compound 318 as a white solid (20 mg, yield 39%). ¹H NMR (400 MHz, CDCl₃) δ: 0.82 (m, 12H), 2.02-2.22 (m, 10H), 2.64 (s, 3H), 2.91 (t, 3H), 3.57-3.81 (m, 10H), 4.22-4.28 (m, 3H), 5.19-5.40 (m, 4H), 6.93-7.79 (m, 8H). MS (ESI) m/z (M+H)⁺ 793.3.

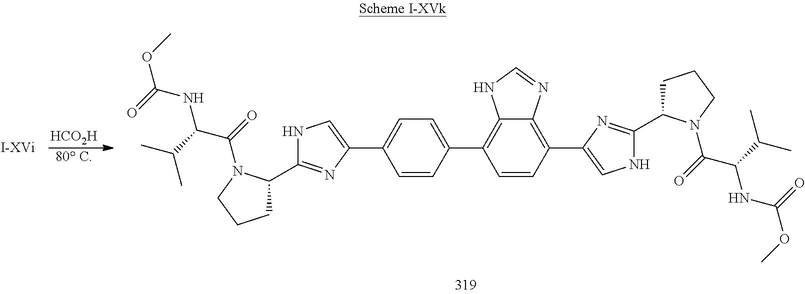

### General Procedure I-EH

A solution of compound I-XVi (50 mg, 0.065 mmol) in formic acid (5 mL) was heated at 70° C. for 1 h. Then the mixture was cooled to r.t. and diluted with water, neutralized with saturated aqueous NaHCO₃, extracted with EtOAc. The combined organic layers were washed with brine, dried over anhydrous Na₂SO₄, and concentrated in vacuo to dryness. The residue was purified by Prep-HPLC to provide compound 319 as a white solid (15 mg, yield 29%). ¹H NMR (400 MHz, CDCl₃) δ: 0.82 (m, 12H), 1.91-2.30 (m, 10H), 2.99 (t, 2H), 2.91 (t, 2H), 3.58-3.82 (m, 10H), 4.26-4.30 (m, 2H), 5.16-5.38 (m, 4H), 7.13-8.12 (m, 8H). MS (ESI) m/z (M+H)⁺ 779.5.

### Example I-XVI

### Preparation of Compound 320

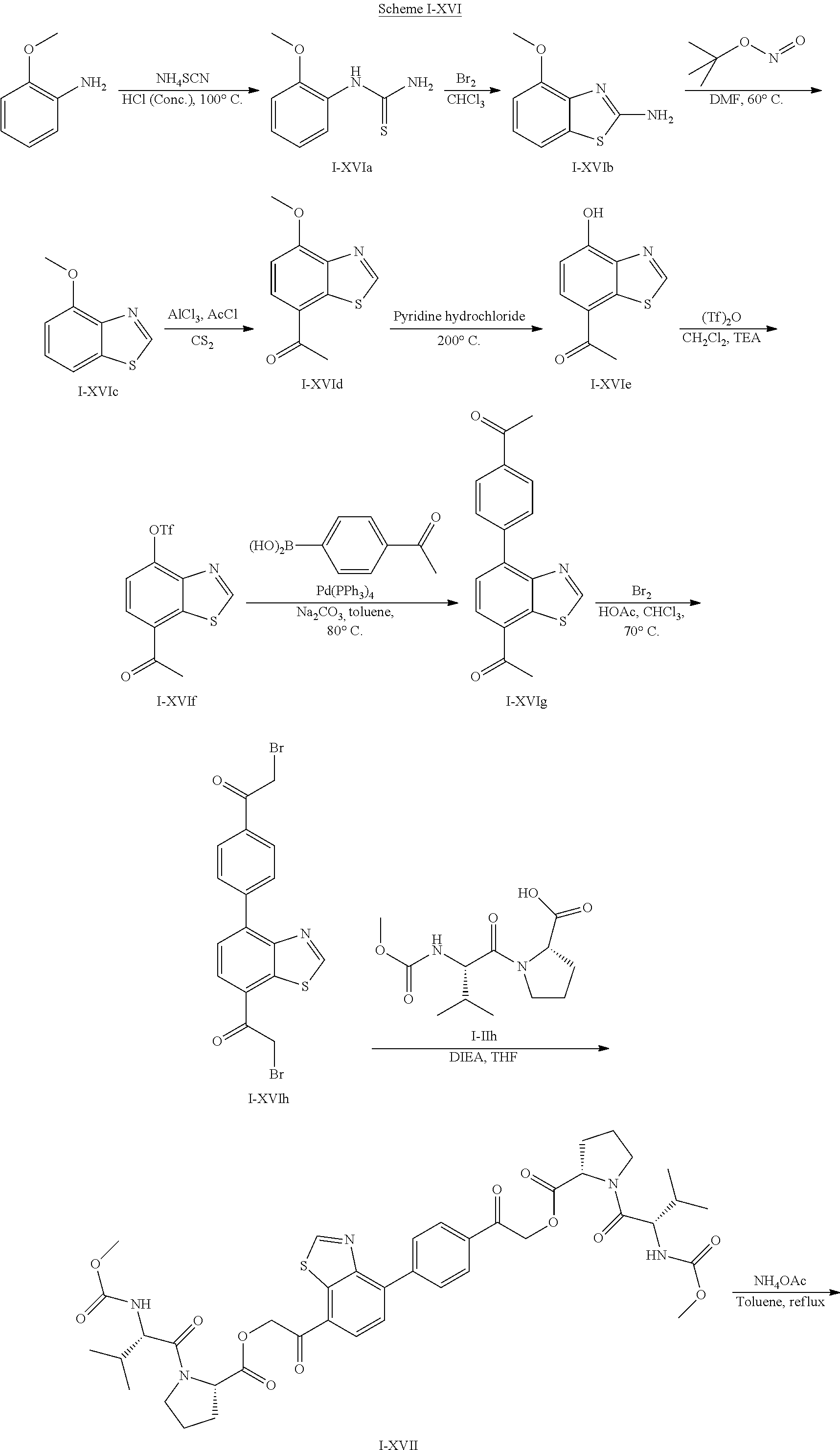

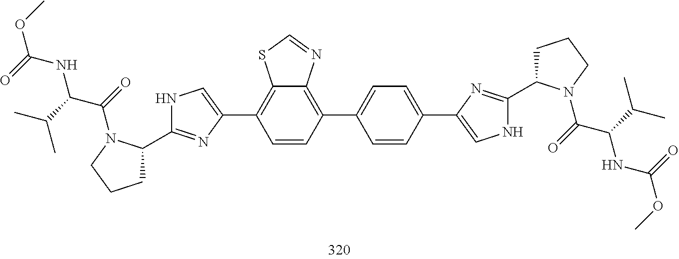

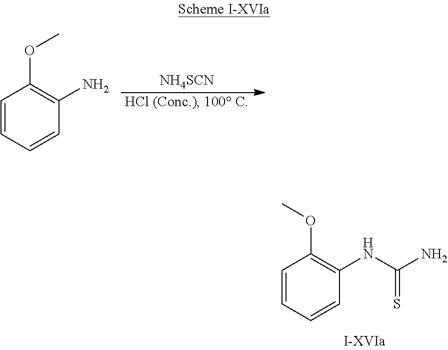

### General Procedure I-EI

Ammonium thiocyanate (NH₄SCN; 3.37 g, 44.3 mmol) was added to a stirred solution of o-anisidine (5.00 g, 44.3 mmol) in aq. HCl (1 M, 45 mL) at 100° C. and the solution stirred at 100° C. for 16 hrs. The solution was diluted with water (60 mL) and the pH value was adjusted to 8 with aqueous ammonia and the mixture was stirred at 5° C. for 2 hrs. The precipitate was filtered, washed with water (5 mL) and ether (5 mL), and dried. The crude solid was purified by column chromatography (petroleum ether/ethyl acetate=4/1), to give compound I-XVIa (1.93 g, yield 24%) as a white powder. MS (ESI) m/z (M+H)⁺ 183.3.

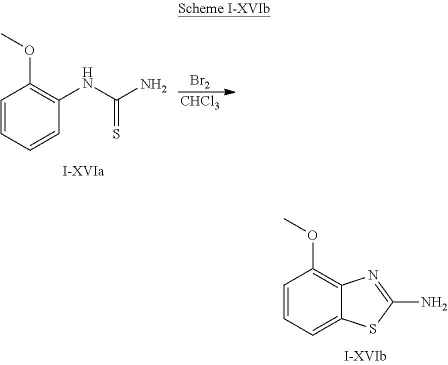

### General Procedure I-EJ

A solution of compound I-XVIa (10 g, 55 mmol) in chloroform (100 mL) was cooled to 10° C. and treated with a solution of bromine (8.8 g, 55 mmol) in chloroform (10 mL). The reaction was stirred at room temperature for 30 min. The resulting suspension was heated at reflux for 30 min. The precipitate was collected via filtration (washed with CH₂Cl₂) to give compound I-XVIb (5 g crude), which was used directly in the next step.

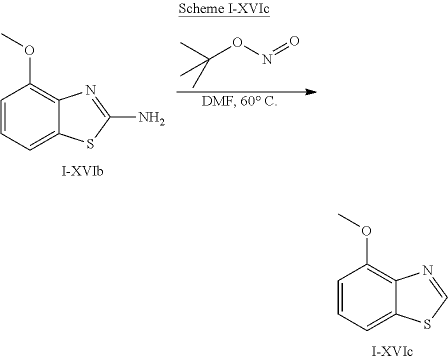

### General Procedure I-EK

Compound I-XVIb (3 g, 16.7 mmol) was diluted with DMF (20 mL), and mixed with tert-butyl nitrite (6.25 g. 60.63 mmol). The resulting mixture was heated at 60° C. for 1 h. After the reaction was completed, the mixture was concentrated. The residue was extracted with ethyl acetate; combined organic extracts were dried over sodium sulfate, filtered and evaporated. Purification by column chromatography on silica gel (petroleum ether/ethyl acetate=7/3) to provide compound I-XVIc (2 g, yield 72%) as a solid. ¹H NMR (400 MHz, CDCl₃) δ 8.83 (s, 1H), 7.47 (d, J=8 Hz, 1H), 7.32 (d, J=8.1 Hz, 1H), 6.87 (d, J=7.6 Hz, 3H), 3.98 (s, 3H). MS (ESI) m/z (M+H)⁺ 165.3.

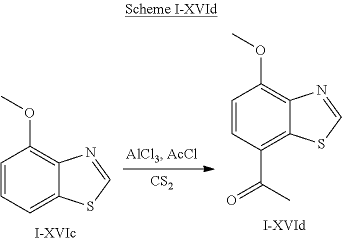

### General Procedure I-EL

A mixture of anhydrous AlCl₃ (1.85 g, 14 mmol) and compound I-XVIc (1 g, 6.0 mmol) in carbon disulfide (CS₂; 10 mL) was heated to reflux for 1 hour. Acetyl chloride (0.5 g, 6.16 mmol) was added and heating was continued for 30 min before evaporation. The mixture was neutralized with aqueous sodium hydrogen carbonate and filtered, and the filtrate was continuously extracted with ethyl acetate. The organic layer was concentrated and then the residue was purified by column chromatography on silica gel (petroleum ether/ethyl acetate=5/1) to afford compound I-XVId (0.5 g, yield 40%). ¹H NMR (300 MHz, CDCl₃) δ 9.06 (s, 1H), 8.08 (d, J=8.4 Hz, 1H), 7.01 (d, J=8.4 Hz, 1H), 4.15 (s, 3H), 2.71 (s, 3H). MS (ESI) m/z (M+H)⁺ 208.3.

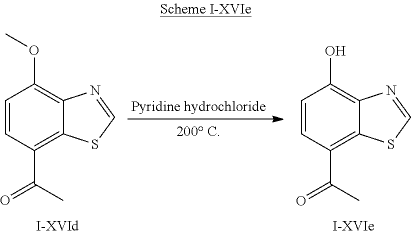

### General Procedure I-EM

A mixture of compound I-XVId (200 mg, 0.97 mmol) in pyridine hydrochloride (5 g) was stirred at 200° C. for 2 hrs. After cooling to r.t., the reaction mixture was poured into ice-water, and then extracted with EtOAc (50 mL×3), the organic layer was washed with brine, dried over sodium sulfate and concentrated. The residue was purified by column chromatography on silica gel to give compound I-XVIe (110 mg, yield 58%). MS (ESI) m/z (M+H)⁺ 194.3.

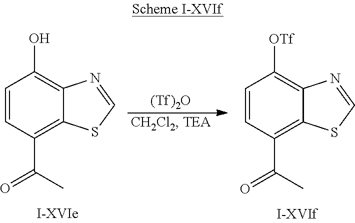

### General Procedure I-EN

Compound I-XVIe (100 mg, 0.48 mmol) was dissolved in anhydrous CH₂Cl₂ (5 mL) in nitrogen atmosphere. Triethylamine (72 mg, 0.72 mmol) was added thereto by one portion. Then the mixture was cooled to 0° C., triflic anhydride (125 mg, 0.6 mmol) was added portion wise. The reaction mixture was stirred at 0° C. for 2 hrs, and then it was diluted with water, extracted with EtOAc (50 mL×3), the organic layer was washed with brine, dried over sodium sulfate and concentrated to provide compound I-XVIf, which was used directly for the next step.

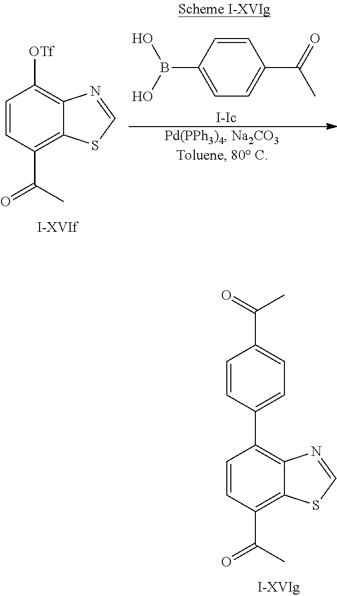

### General Procedure I-EO

To a solution of compound I-XVIf (120 mg, 0.35 mmol) in toluene (5 mL), Na₂CO₃ (53 mg, 0.5 mmol) and 4-acetylphenylboronic acid (I-IC; 82 mg, 0.4 mmol) were added, then the reaction flask was purged with nitrogen, then Pd (PPh₃)₄ (12 mg, 0.01 mmol) was added, the resulting mixture was stirred at 80° C. overnight under nitrogen atmosphere. After the reaction was completed, the reaction mixture was poured into water, extracted with EtOAc (50 mL×3). The combined organic layers were washed with brine, dried over sodium sulfate and concentrated. The residue was purified by column chromatography on silica gel (PE:EtOAc=2:1) to provide compound I-XVIg (100 mg, 83% yield over two steps). ¹H NMR (400 MHz, DMSO-d6): δ 9.65 (s, 1H), 8.50 (d, J=8.0 Hz, 1H), 8.17 (m, 4H), 8.00 (d, J=7.6 Hz, 1H), 2.87 (s, 3H), 2.72 (s, 3H). MS (ESI) m/z (M+H)⁺ 296.3.

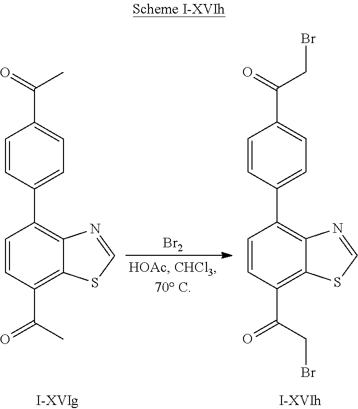

### General Procedure I-EP

Compound I-XVIg (100 mg, 0.32 mmol) was dissolved in CHCl₃ (2.5 mL) and acetic acid (2.5 mL), the mixture was stirred at 70° C., then bromine (202 mg, 1.28 mmol) was added dropwise. After the reaction was completed, the mixture was poured into water, extracted with EtOAc (50 mL×3). The combined organic layers were washed with brine, dried over sodium sulfate and concentrated. The product I-XVIh was used directly in the next step without further purification.

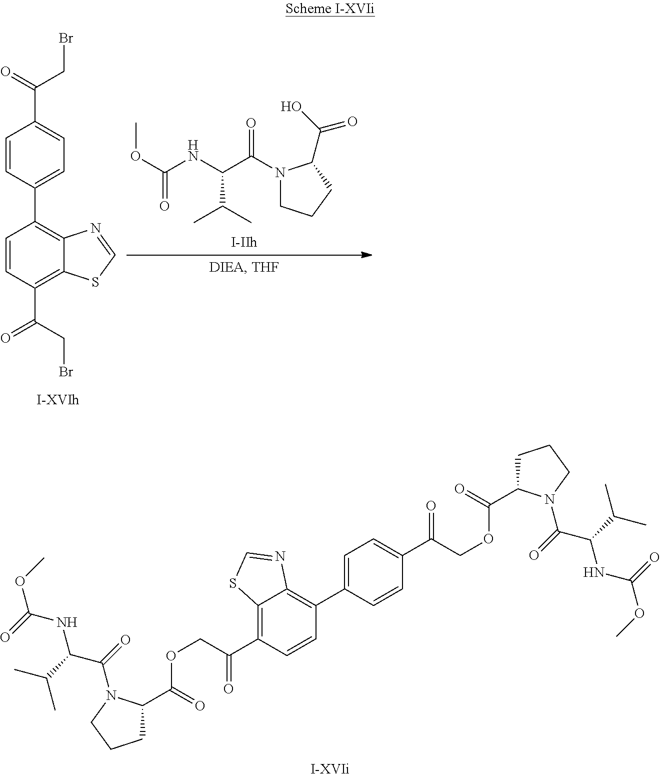

### General Procedure I-EQ

Diisopropylethylamine (83 mg, 0.64 mmol) and compound I-IIh (174 mg, 0.64 mmol) were added to a suspension of compound I-XVIh (149 mg, 0.32 mmol) in THF (5 mL). The resulting mixture was stirred for 1 h as the solids dissolved. The reaction mixture was quenched by the addition of 13% aqueous sodium chloride (20 mL). The layers were separated, and the organic layer was concentrated, and purified by column chromatography on silica gel (PE:EtOAc=1:1) to obtain compound I-XVIi (20 mg, yield 8%). MS (ESI) m/z (M+H)⁺ 836.2.

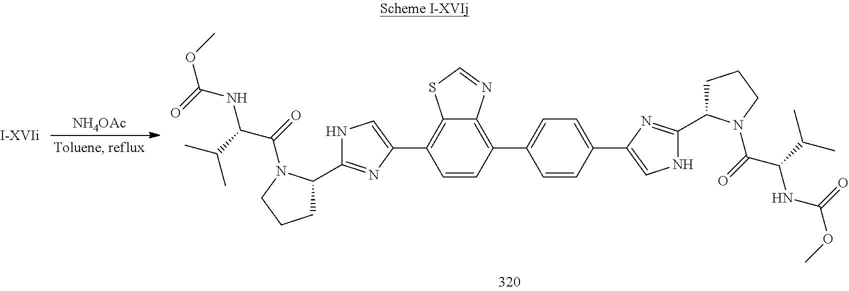

### General Procedure I-ER

To a solution of compound I-XVIi (20 mg, 0.024 mmol) in toluene (10 mL) was added ammonium acetate (5 g, 65 mmol) and heated to 100° C. overnight. LCMS indicated the reaction was completed, and then the mixture was cooled to r.t. and concentrated in vacuo. The residue was purified by Prep-HPLC to provide compound 320 (7 mg, yield 42%). ¹H NMR (400 MHz, CDCl₃): δ 10.57 (m, 1H), 9.03 (s, 1H), 7.80-7.63 (m, 8H), 5.40-7.38 (m, 2H), 5.26-5.21 (m, 2H), 4.27 (m, 2H), 3.77 (m, 2H), 3.63 (m, 8H), 2.42 (m, 2H), 2.50-1.85 (m, 9H), 0.85 (m, 12H). MS (ESI) m/z (M+H)⁺ 796.3.

### Example I-XVII

### Preparation of Compound 321

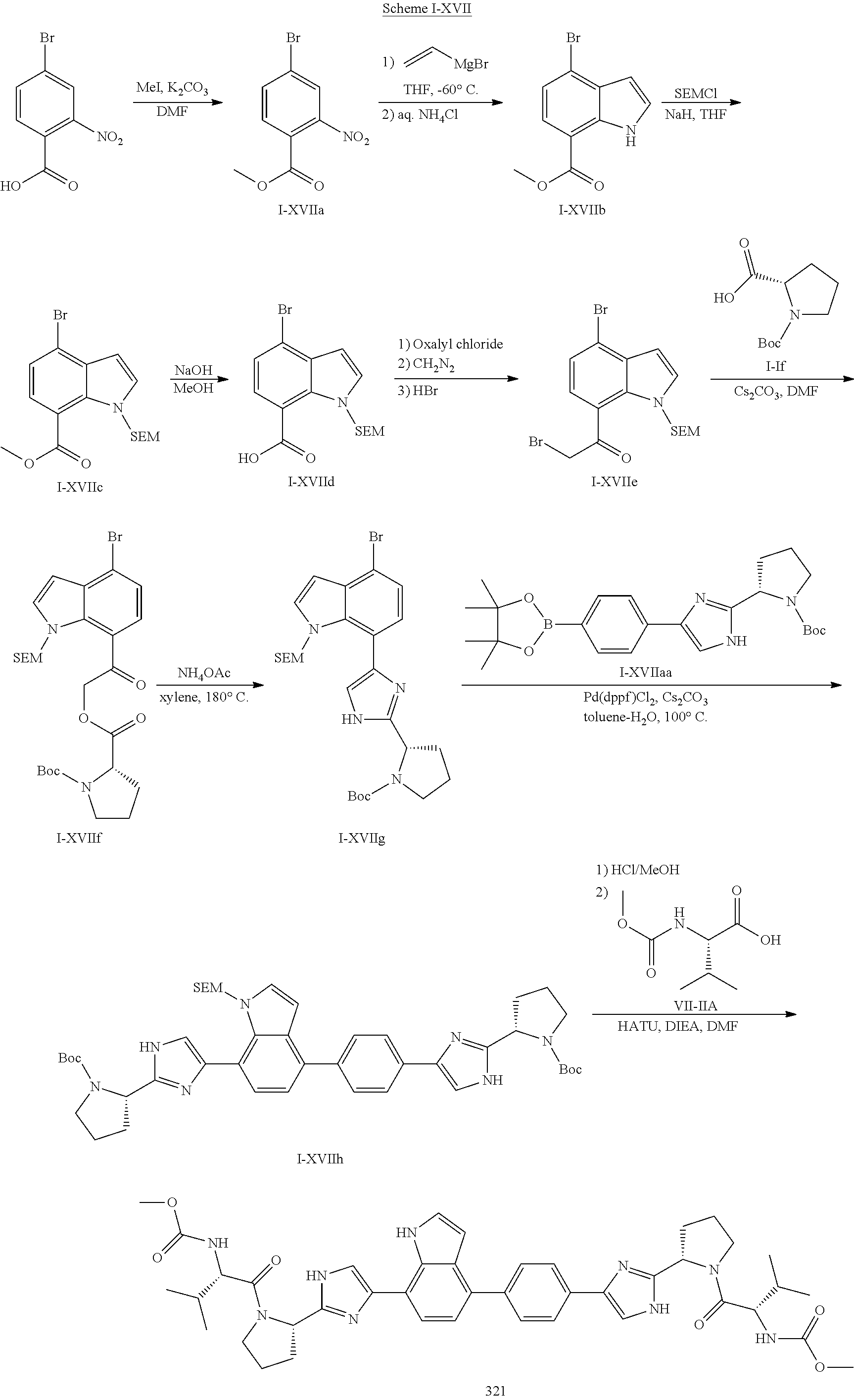

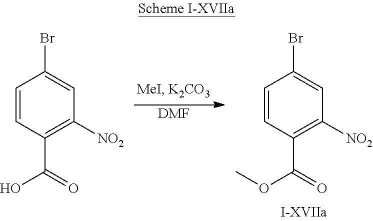

### General Procedure I-ES

To a mixture of 4-bromo-2-nitrobenzoic acid (10 g, 41 mmol) and K₂CO₃ (11.3 g, 82 mmol) in 100 mL of DMF was added CH₃I (7.1 g, 50 mmol) dropwise and the mixture was stirred at 80° C. for 3 hrs. After cooling to r.t, the mixture was filtered, the filtrated was concentrated under reduced pressure to remove DMF, and the residue was dissolved with EtOAc (50 mL), washed with water (50 mL), brine (50 mL), dried over anhydrous Na₂SO₄ and concentrated in vacuo. The crude product was purified by column chromatography to give methyl 4-bromo-2-nitrobenzoate (I-XVIIa, 10 g, yield 94%). ¹H NMR (400 MHz, CDCl₃) δ 8.02 (s, 1H), 7.81 (d, J=8.0 Hz, 1H), 7.66 (d, J=8.0 Hz, 1H), 3.92 (s, 3H).

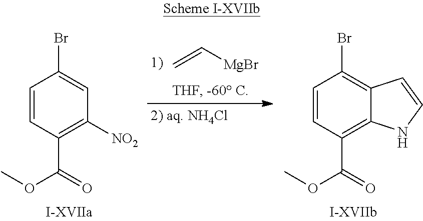

### General Procedure I-ET

To a solution of methyl 4-bromo-2-nitrobenzoate (I-XVIIa, 5 g, 19 mmol) in 30 mL of dry THF was added vinylmagnesium bromide (1.0 M in THF, 48 mL, 48 mmol) dropwise at −60° C. under Nitrogen. The reaction mixture was stirred at room temperature overnight. Then the mixture was treated with saturated aq. NH₄Cl, the resulting mixture was extracted with EtOAc (50 mL×2), the organic phase was washed with water (100 mL), brine (100 mL), dried over anhydrous Na₂SO₄, and concentrated. The residue was purified by column chromatography to afford compound I-XVIIb (1.5 g, yield 31%). ¹H NMR (400 MHz, CDCl₃) δ 9.90 (s, 1H), 7.66 (d, J=8.0 Hz, 1H), 7.52-7.30 (m, 2H), 6.58 (t, J=2.8 Hz, 1H), 3.91 (s, 3H).

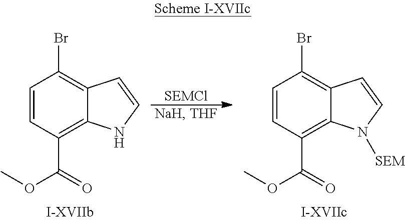

### General Procedure I-EU

Sodium hydride (NaH, 60% dispersion in mineral oil, 0.36 g, 9.0 mmol) was added to a mixture of compound I-XVIIb (1.5 g, 6.0 mmol) in 20 mL of dry THF, the mixture was stirred at 0° C. for 30 min. Then 2-(trimethylsilyl)ethoxymethyl chloride (SEMCl, 1.2 g, 7.2 mmol) was added dropwise at 0° C. under Nitrogen. The resulting mixture was stirred at room temperature for 1 hour. Then treated with water, and extracted with EtOAc (50 mL×3), the organic phase was washed with water (20 mL), brine (20 mL), dried over anhydrous Na₂SO₄, and concentrated under reduced pressure. The residue was purified by column chromatography to afford compound I-XVIIc (1.6 g, yield 70%). ¹H NMR (400 MHz, CDCl₃) δ 7.61 (d, J=8.0 Hz, 1H), 7.44 (d, J=8.0 Hz, 1H), 7.36 (d, J=4.0 Hz, 1H), 6.76 (d, J=4.0 Hz, 1H), 5.80 (s, 2H), 4.06 (s, 3H), 3.30 (t, J=8.0 Hz, 2H), 0.87 (t, J=8.0 Hz, 2H), 0.00 (s, 9H).

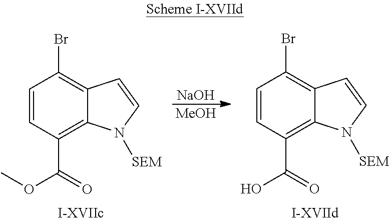

### General Procedure I-EV

A mixture of compound I-XVIIc (0.3 g, 0.28 mmol) and NaOH/MeOH (2 M, 5 mL) in 5 mL of MeOH was stirred at 60° C. for 5 hrs. After being cooled to r.t., the mixture was acidified to pH 2˜3 by addition of aq. HCl (2 N) and extracted with DCM (20 mL×3). The combined organic layers were dried over Na₂SO₄ and concentrated to afford compound I-XVIId (0.25 g, yield 87%). ¹H NMR (300 MHz, CDCl₃) δ 7.82 (d, J=8.0 Hz, 1H), 7.50 (d, J=8.0 Hz, 1H), 7.41 (d, J=3.2 Hz, 1H), 6.82 (d, J=3.6 Hz, 1H), 5.91 (s, 2H), 3.34 (t, J=8.0 Hz, 2H), 0.90 (t, J=8.0 Hz, 2H), 0.00 (s, 9H).

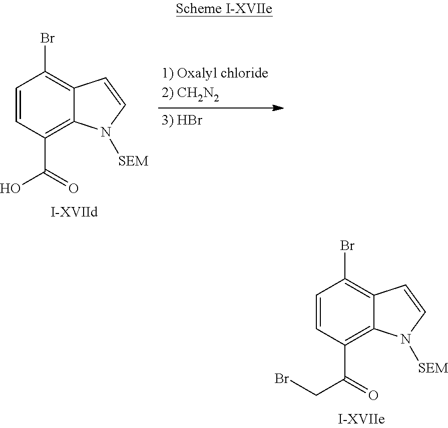

### General Procedure I-EW

To a solution of compound I-XVIId (1.3 g, 3.5 mmol) in 20 mL of dry DCM was added oxalyl chloride (0.7 g, 5.3 mmol) and the mixture stirred at room temperature for 2 hours. After concentration under reduced pressure, the residue was dissolved in 10 mL of dry DCM, the solution was added dropwise to a solution of diazomethane in Et₂O (1 M, 20 mL, 20 mmol) at −10° C. under nitrogen. The reaction mixture was stirred at room temperature for 3 hours. Then 10 mL of aq. HBr (40%) was added dropwise and the mixture was stirred for another 1 hour. After the reaction was completed, the mixture was washed with aq. NaHCO₃ (50 mL), water (50 mL), brine (50 mL), and then the organic layer was dried over anhydrous Na₂SO₄, and concentrated under reduced pressure, the residue was purified by column chromatography to afford compound I-XVIIe (1.0 g, yield 63%). ¹H NMR (300 MHz, CDCl₃): δ 7.46-7.38 (m, 2H), 7.33 (s, 1H), 6.74 (d, J=3.6 Hz, 1H), 5.52 (s, 2H), 4.66 (s, 2H), 3.27 (t, J=8.4 Hz, 2H), 0.85 (t, J=8.4 Hz, 2H), 0.00 (s, 9H).

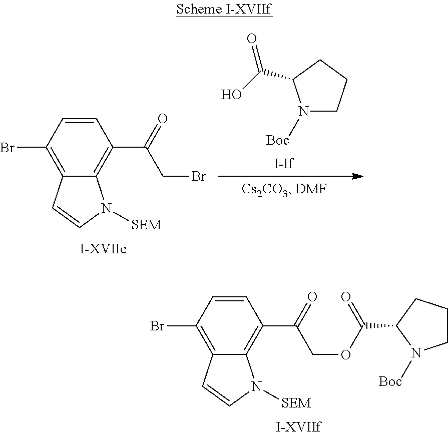

### General Procedure I-EY

The mixture of compound I-XVIIe (280 mg, 0.63 mmol), N-Boc-proline (I-If; 135 mg, 0.63 mmol) and Cs₂CO₃ (295 mg, 0.9 mmol) in 10 mL of DMF was stirred at room temperature for 2 hours. Then the mixture was diluted with EtOAc (10 mL), washed with water (20 mL), brine (50 mL), dried over anhydrous Na₂SO₄, and concentrated under reduced pressure. The residue was purified by column chromatography to give compound I-XVIIf (170 mg, yield 50%). MS (ESI) m/z (M+H)⁺ 581.3.

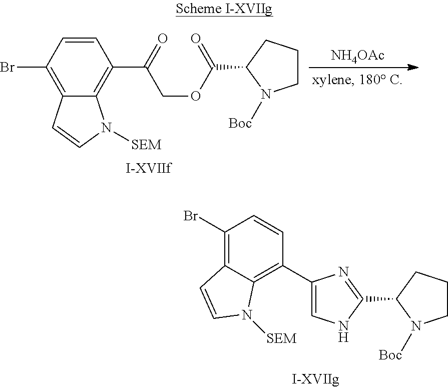

### General Procedure I-EZ

A mixture of compound I-XVIIf (170 mg, 0.3 mmol) and NH₄OAc (230 mg, 3 mmol) in 20 mL of xylene was stirred at 180° C. for 5 hours, in a sealed tube. After being cooled to r.t., the mixture was diluted with EtOAc (20 mL), washed with water (30 mL), the organic layers was dried over anhydrous Na₂SO₄ and concentrated under reduced pressure. The residue was purified by column chromatography to afford compound I-XVIIg (100 mg, yield 63%). ¹H NMR (400 MHz, CDCl₃): δ 7.40-7.10 (m, 4H), 6.47 (d, J=3.2 Hz, 1H), 5.54-5.45 (m, 2H), 5.12-5.10 (m, 1H), 3.54-3.52 (m, 2H), 3.31-3.29 (m, 2H), 3.01-2.99 (m, 1H), 2.30-2.04 (m, 4H), 1.59 (m, 9H), 0.90-0.84 (m, 2H), 0.00 (s, 9H).

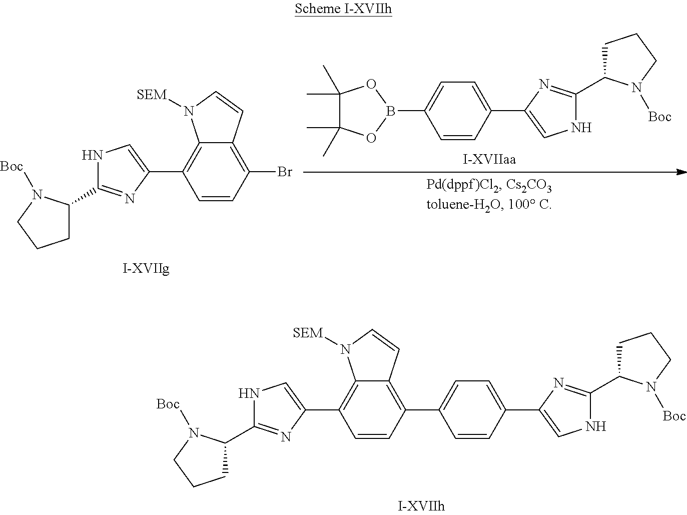

### General Procedure I-FA

To a flask were added compound I-XVIIg (200 mg, 0.36 mmol), compound I-XVIIaa (172 mg, 0.39 mmol), Pd(dppf)Cl₂ (10% mol) and Cs₂CO₃ (231 mg, 0.72 mmol) in toluene/water (10 mL/1 mL). The reaction mixture was stirred at 100° C. for 2 hours. After being cooled to r.t., the mixture was diluted with EtOAc (20 mL), the organic layers was washed with water (30 mL), dried over anhydrous Na₂SO₄, and concentrated under reduced pressure. The residue was purified by column chromatography to give compound I-XVIIh (170 mg, yield 61%). MS (ESI) m/z (M+H)⁺ 794.3.

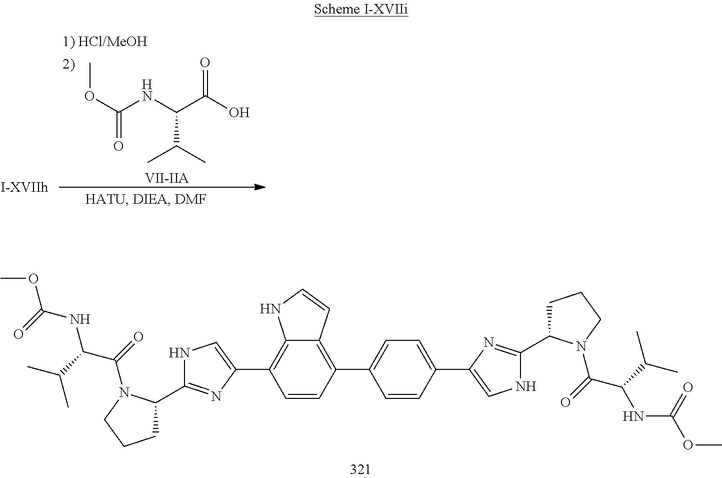

### General Procedure I-FB

The mixture of compound I-XVIIh (100 mg, 0.13 mmol) in 10 mL of HCl/MeOH (4 N) was stirred at 60° C. for 3 hours. After concentration under reduced pressure, the residue was dissolved in 10 mL of DMF. Then compound VII-IIA (44 mg, 0.26 mmol), HATU (100 mg, 0.26 mmol) and DMA (52 mg, 0.4 mmol) were added; the reaction mixture was stirred at room temperature for 5 hours. EtOAc (50 mL) was added, washed with water (10 mL×3), the organic layer was concentrated and purified by prep-HPLC to afford compound 321 (23 mg, yield 23%). ¹H NMR (400 MHz, CD₃OD): δ 7.91-7.74 (m, 4H), 7.49-7.40 (m, 4H), 7.17 (s, 1H), 6.71 (s, 1H), 5.41-5.19 (m, 2H), 4.31-4.20 (m, 2H), 4.09-3.82 (m, 4H), 3.72-3.50 (m, 6H), 2.40-2.22 (m, 5H), 2.12-2.04 (m, 5H), 0.99-0.93 (m, 12H).

### Example I-XVIII

### Preparation of Compound 322

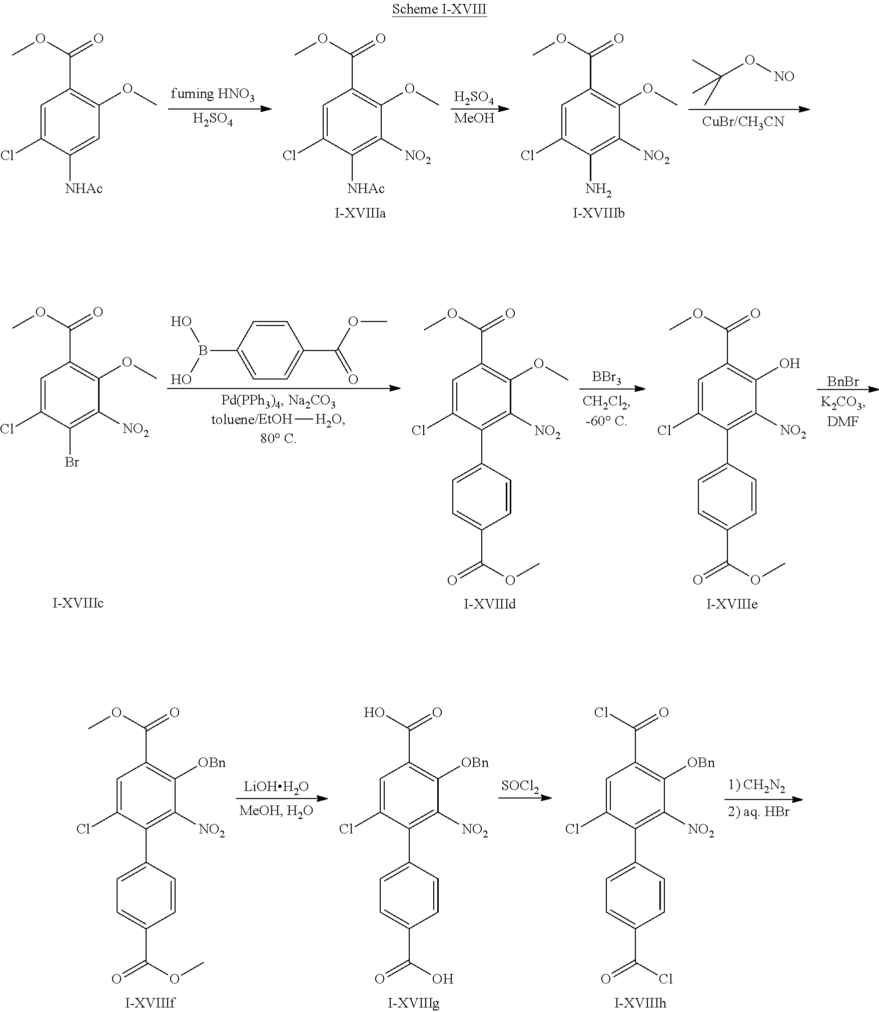

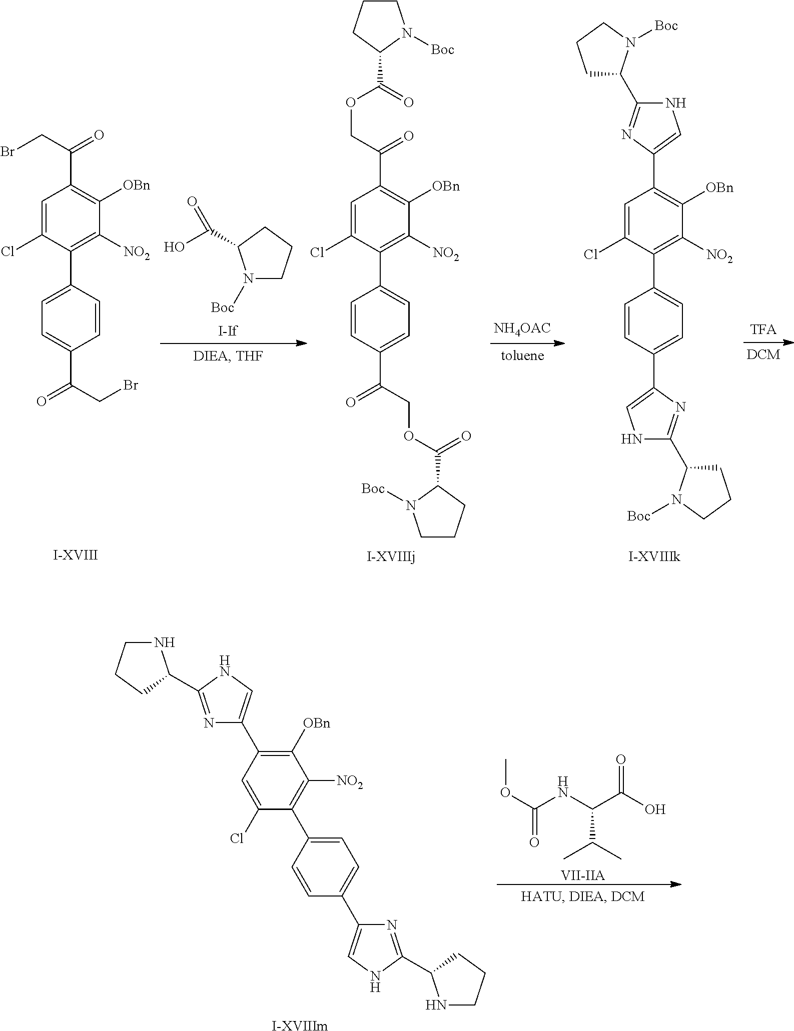

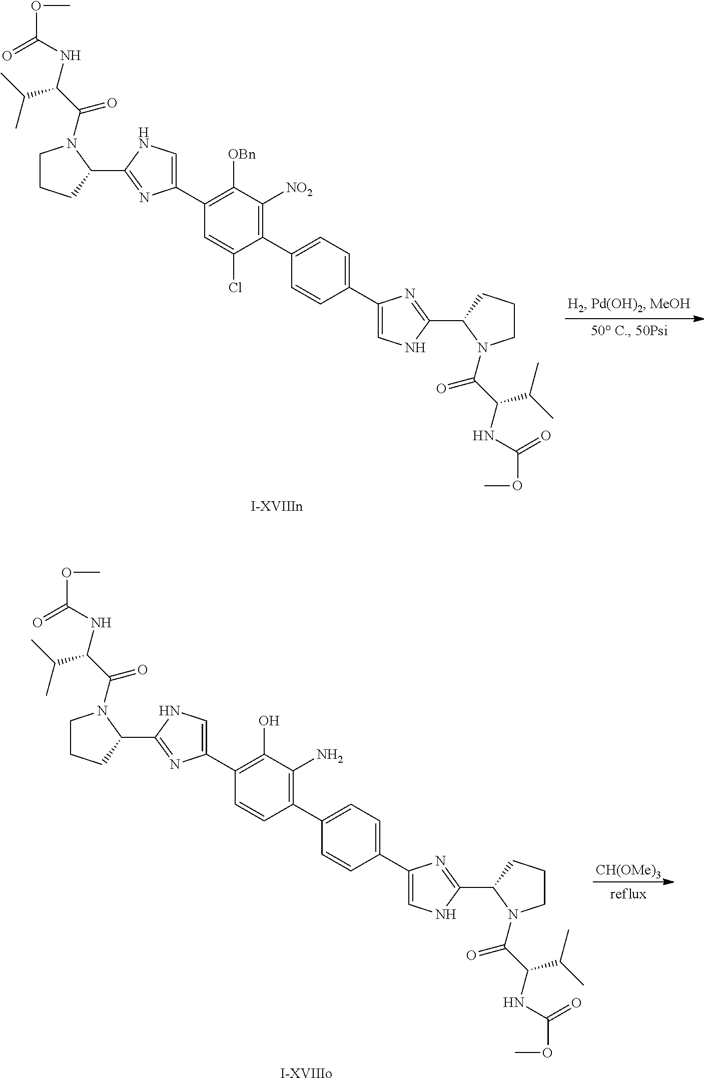

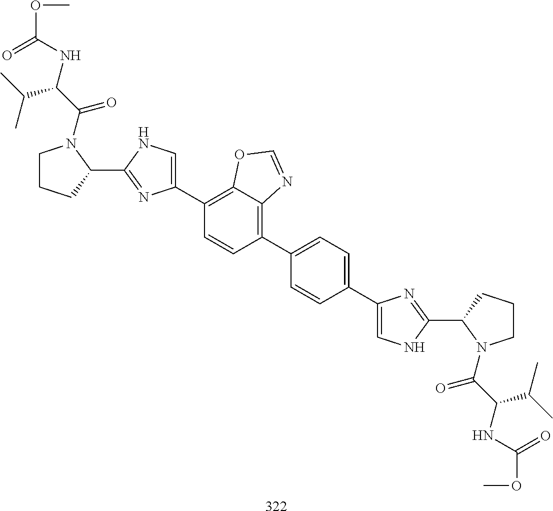

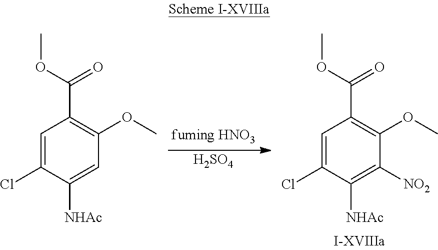

### General Procedure I-FC

Methyl 4-acetamido-5-chloro-2-methoxybenzoate (20 g, 77.8 mmol) was added into 150 mL of conc. H₂SO₄ at 0° C. in portions. 50 mL of fuming HNO₃ in 50 mL of conc. H₂SO₄ was added thereto. The mixture was stirred at 0° C. for 1 h. The mixture was poured into 300 mL of ice water. The solid formed was filtered and washed by ice water and dried to give compound I-XVIIIa (15 g, yield 64%) as pale-yellow solid. ¹H NMR (300 MHz, CDCl₃) δ 8.11 (s, 1H), 4.00 (s, 3H), 3.96 (s, 3H), 2.22 (s, 3H).

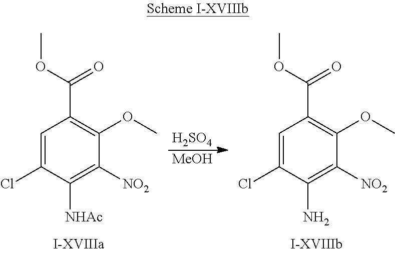

### General Procedure I-FD

To a solution of compound I-XVIIIa (15.0 g, 49.7 mmol) in 100 mL of MeOH was added 6 mL of conc. H₂SO₄. The solution was heated to reflux for 7 hrs. Subsequently, the solution was concentrated under reduced pressure. The residue was diluted with water (30 mL), and then neutralized by addition of saturated aq. NaHCO₃, the solution was extracted with EtOAc (30 mL×3), the organic layer was washed with water and brine, dried over anhydrous Na₂SO₄, and concentrated in vacuo to give compound I-XVIIIb (12.8 g, yield 99%) as yellow solid.

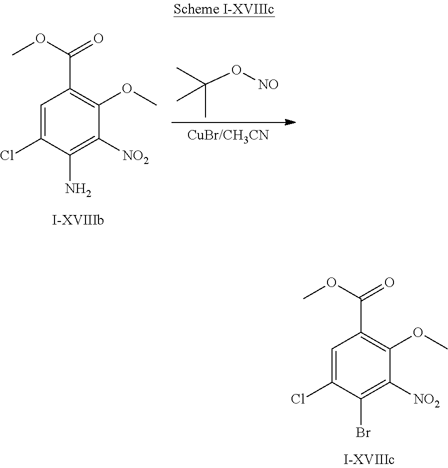

### General Procedure I-FE

To a solution of CuBr (16.3 g, 72.6 mmol) in 100 mL of CH₃CN was added t-butyl nitrite (6.494 g, 63.1 mmol). A solution of compound I-XVIIIb (12.6 g, 48.5 mmol) in 100 mL of CH₃CN was added dropwise into the above solution at 70° C. The mixture was stirred at 70° C.˜0.80° C. for 4 hrs. The solution was concentrated under reduced pressure. The residue was added into 100 mL of aqueous ammonia (10%), followed by extraction with EtOAc (30 mL×3), the organic layer was washed with water and brine, dried over anhydrous Na₂SO₄, and concentrated in vacuo The residue was purified by column chromatography on silica gel (PE/EA 100:1-50:1) to give compound I-XVIIIc (12.4 g, yield 80%) as yellow solid.

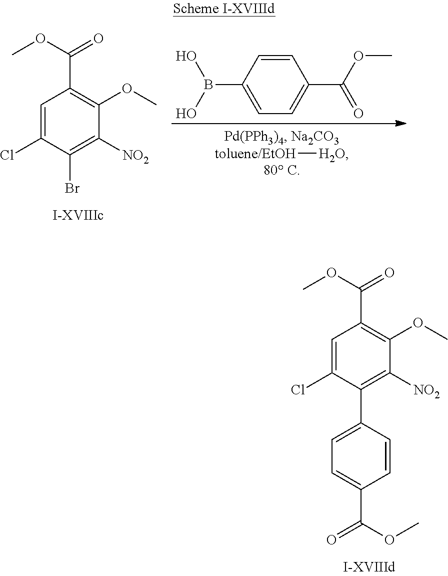

### General Procedure I-FF

To a solution of compound I-XVIIIc (5.0 g, 15.43 mmol) in 150 mL of toluene was added 4-(methoxycarbonyl)phenylboronic acid (3.055 g, 17.0 mmol), Na₂CO₃ (1.962 g, 18.52 mmol), EtOH (15 mL), H₂O (9 mL) and Pd (PPh₃)₄ (0.891 g, 0.77 mmol) under nitrogen. The solution was stirred at 80° C. overnight. After cooling to r.t., the mixture was extracted with EtOAc (100 mL×3), the organic layer was washed with water and brine, dried over anhydrous Na₂SO₄, and concentrated in vacuo, the residue was purified by column chromatography on silica gel (PE/EA gradient 100:1-80:1-50:1-25:1) to give compound I-XVIIId (1.25 g, yield 21%) as light-yellow solid.

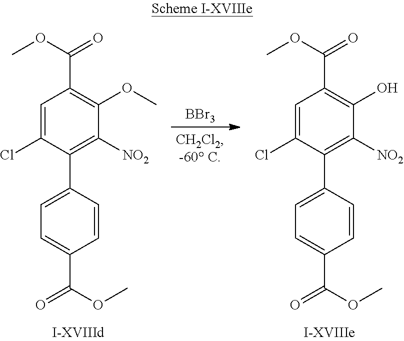

### General Procedure I-FG

Boron tribromide (BBr₃, 1.383 g, 5.54 mmol) was added into a solution of compound I-XVIIId (300 mg, 0.792 mmol) in 8 mL of anhydrous DCM at −60° C.˜−70° C. The mixture was stirred at −60° C.˜−70° C. for 2 hrs. TLC (PE/EA 3:1) showed disappearance of compound I-XVIIId indicating completion of the reaction. The mixture was quenched by ice water, extracted with EtOAc (10 mL×3), the organic layer was washed with water and brine, dried over anhydrous Na₂SO₄, and concentrated in vacuo to give compound I-XVIIIe (250 mg, yield 90%) as white solid.

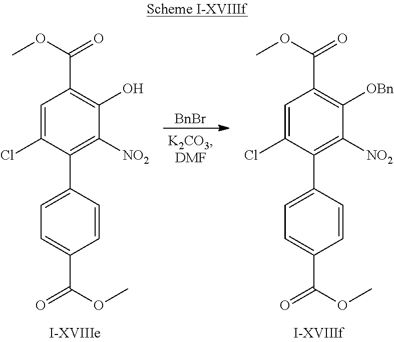

### General Procedure I-FH

Benzyl bromide (36 mg, 0.22 mmol) in 1 mL of DMF was added into a solution of compound I-XVIIIe (50 mg, 0.142 mmol) and K₂CO₃ (30 mg, 0.22 mmol) in 4 mL of DMF. The resulting mixture was stirred at 40° C. overnight, quenched with water, extracted with EtOAc (15 mL×3), the organic layer was washed with water and brine, dried over anhydrous Na₂SO₄, and concentrated in vacuo, the residue was purified by prep-TLC (PE/EA=5:1) to afford compound I-XVIIIf (25 mg, yield 39%) as light yellow solid. ¹H NMR (300 MHz, CDCl₃) δ 8.15-8.11 (m, 3H), 7.41-7.38 (m, 7H), 5.15 (s, 2H), 3.95 (s, 6H).

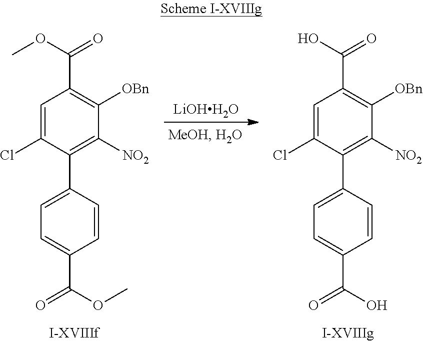

### General Procedure I-FI

Compound I-XVIIIf (170 mg, 0.37 mmol) and LiOH monohydrate (78 mg, 1.85 mmol) was added into 3 mL of THF/H₂O (2:1). The mixture was stirred at r.t. overnight. The mixture was acidified with aq. HCl (1 M), and extracted with EtOAc (5 mL×3), the organic layer was washed with water and brine, dried over anhydrous Na₂SO₄, and concentrated in vacuo to afford compound I-XVIIIg (157 mg, yield 99%). ¹H NMR (300 MHz, DMSO-d₆) δ 8.20 (s, 1H), 8.05 (d, J=8.4 Hz, 2H), 7.48 (d, J=8.1 Hz, 2H), 7.39-7.36 (m, 5H), 5.13 (s, 2H).

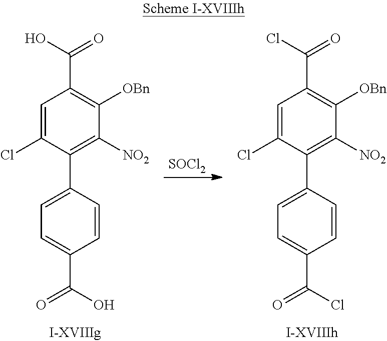

### General Procedure I-FJ

Compound I-XVIIIg (100 mg, 0.234 mmol) was added into 2 mL of SOCl₂. The mixture was heated to reflux for 2 hrs. After that, the mixture was concentrated in vacuo to afford compound I-XVIIIh, which was used in next step directly.

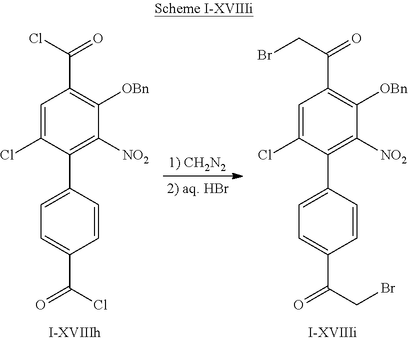

### General Procedure I-FK

To a solution of compound I-XVIIIh (108.7 mg, 0.234 mmol) in 2 mL of DCM was added a solution of diazomethane in ether (0.7 M, 1.4 mL, 1 mmol) at −5° C., the solution was stirred at r.t. for 1 h, 2 mL of aq. HBr (40%) was added into the solution at −5° C., and then the mixture was stirred at r.t overnight. The reaction mixture was adjusted to pH=7 by addition of saturated aq. NaHCO₃, the organic layer was separated, dried over anhydrous Na₂SO₄ and concentrated under reduced pressure to afford compound I-XVIIIi (130 mg, yield 95%) as yellow solid. ¹H NMR (400 MHz, CDCl₃) δ 8.10 (d, J=7.6 Hz, 2H), 7.84 (s, 1H), 7.47 (d, J=8.4 Hz, 2H), 7.41-7.39 (m, 3H), 7.32 (m, 2H), 5.05 (s, 2H), 4.48 (s, 2H), 4.36 (s, 1H).

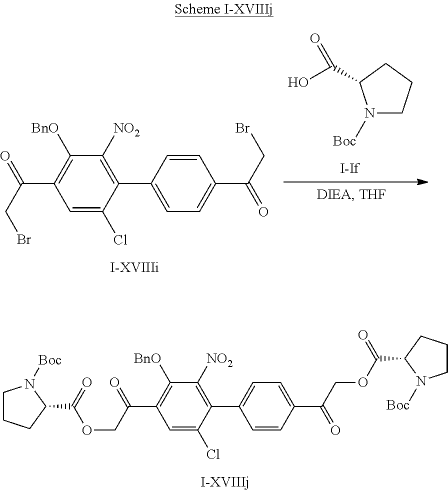

### General Procedure I-FL

Compound I-XVIIIi (130 mg, 0.224 mmol), N-Boc-proline (I-If, 192 mg, 0.895 mmol) and DMA (144.5 mg, 1.12 mmol) were added into 3 mL of THF. The mixture was stirred overnight at r.t. TLC (PE/EA 3:1) showed disappearance of compound I-XVIIIi indicating completion of the reaction. The solution was quenched with water, extracted with EtOAc (30 mL×3), the organic layer was washed with water and brine, dried over anhydrous Na₂SO₄, and concentrated in vacuo, the residue was purified by prep-TLC (PE/EA 3:1) to afford compound I-XVIIIj (74 mg, yield 40%) as white solid. ¹H NMR (400 MHz, CDCl₃) δ 8.01-7.98 (m, 2H), 7.91-7.87 (m, 1H), 7.46 (m, 2H), 7.44 (m, 5H), 5.60-5.02 (m, 2H), 4.51-4.41 (m, 4H), 4.41-4.35 (m, 2H), 3.61-3.18 (m, 4H), 2.34-2.24 (m, 4H), 2.11-1.90 (m, 2H), 1.51-1.27 (m, 13H), 1.24-1.13 (m, 5H).

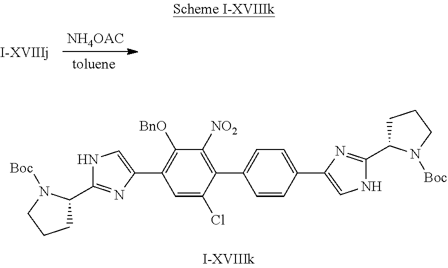

### General Procedure I-FM

Compound I-XVIIIj (70 mg, 0.082 mmol) and NH₄OAc (63 mg, 0.83 mmol) was added into 3 mL of toluene. The mixture was heated to reflux overnight. After cooling to r.t., water was added (20 mL), extracted with EtOAc (30 mL×3), the organic layer was washed with water and brine, dried over anhydrous Na₂SO₄, and concentrated in vacuo, the residue was purified by prep-TLC (PE/EA 3:1) to provide compound I-XVIIIk (37.5 mg, yield 57%) as white solid. ¹H NMR (300 MHz, CDCl₃) δ 10.79-10.43 (m, 1H), 8.36 (s, 1H), 7.83-7.81 (m, 2H), 7.72-7.70 (m, 1H), 7.35-7.32 (m, 6H), 7.28 (s, 1H), 4.98 (m, 4H), 3.49-3.42 (m, 4H), 3.04-3.99 (m, 2H), 2.31-2.25 (m, 4H), 2.11-1.99 (m, 2H), 1.49 (s, 18H). MS (ESI) m/z [M+H]⁺ 810.2

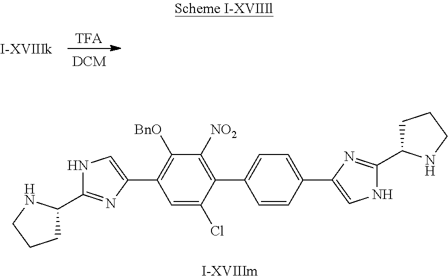

### General Procedure I-FN

To a solution of compound I-XVIIIk (325 mg, 0.401 mmol) in 3 mL of DCM was added TFA (1.0 mL). The mixture was stirred at r.t for 4 hrs. While the reaction was completed, the solution was concentrated in vacuo to give compound I-XVIIIm, which was used for next step directly. MS (ESI) m/z [M+H]⁺ 609.9.

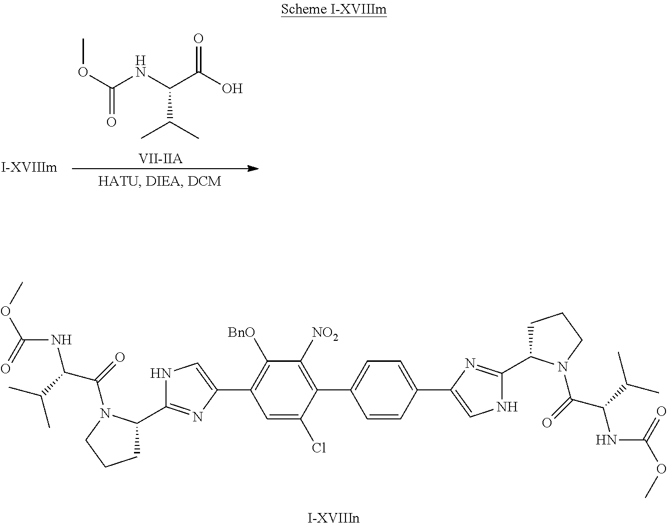

### General Procedure I-FO

To a solution of compound I-XVIIIm (426 mg, 0.40 mmol) in 12 mL of CH₂Cl₂ was added DIEA (420 mg, 3.2 mmol), compound VII-IIa (280 mg, 1.6 mmol) and HATU (396 mg, 1.043 mmol). The reaction solution was stirred at r.t. overnight. The mixture was diluted with CH₂Cl₂ (50 mL), washed with water (10 mL×3) and brine, the organic layer was dried over anhydrous Na₂SO₄, and concentrated in vacuo to afford crude compound I-XVIIIn (300 mg, yield 81%). MS (ESI) m/z [M+H]⁺ 924.3.

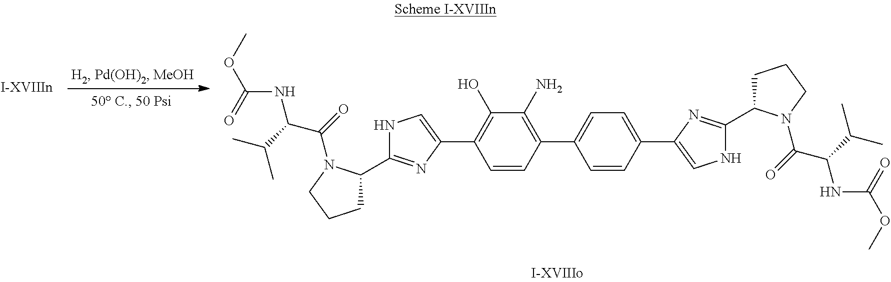

### General Procedure I-FP

To a mixture of Pd(OH)₂ (30 mg) in 20 mL of MeOH was added a solution of compound I-XVIIIn (300 mg, 0.325 mmol) in 30 mL of MeOH. The mixture was stirred at 50° C. under hydrogen (pressure 50 Psi) for 1 day. The solution was filtered and the solid was washed with MeOH. The filtrate was concentrated under reduced pressure to give compound I-XVIIIo (210 mg, yield 84%). MS (ESI) m/z [M]⁺ 769.4.

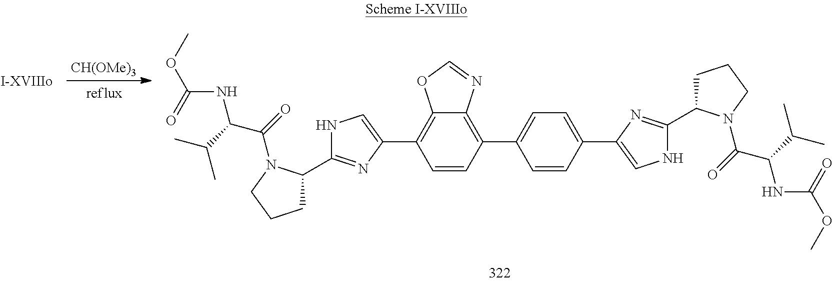

### General Procedure I-FQ

A mixture of compound I-XVIIIo (200 mg, 0.26 mmol) and 10 mL of trimethyl ortho-formate was heated to reflux overnight. After cooling to r.t, the mixture was concentrated under reduced pressure and purified by prep-TLC (DCM/MeOH 10:1) to afford compound 322 (11.2 mg, yield 5.5%). ¹H NMR (400 MHz, CDCl₃) δ 8.21-8.18 (m, 1H), 8.01-7.85 (m, 3H), 7.67-7.51 (m, 4H), 5.42-5.32 (m, 2H), 5.28-2.27 (m, 2H), 4.39-4.36 (m, 2H), 4.39-4.33 (m, 2H), 3.87-3.68 (m, 8H). MS (ESI) m/z [M+H]⁺ 780.3.

### Example I-XIX

### Preparation of Compound 323

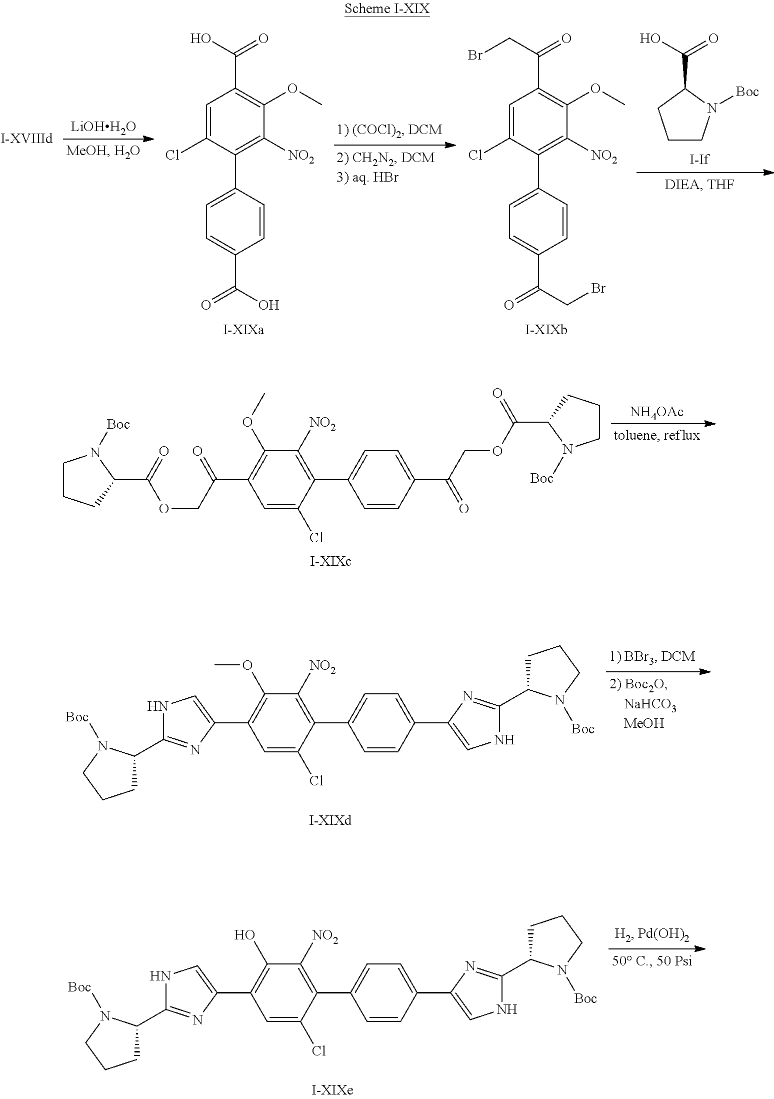

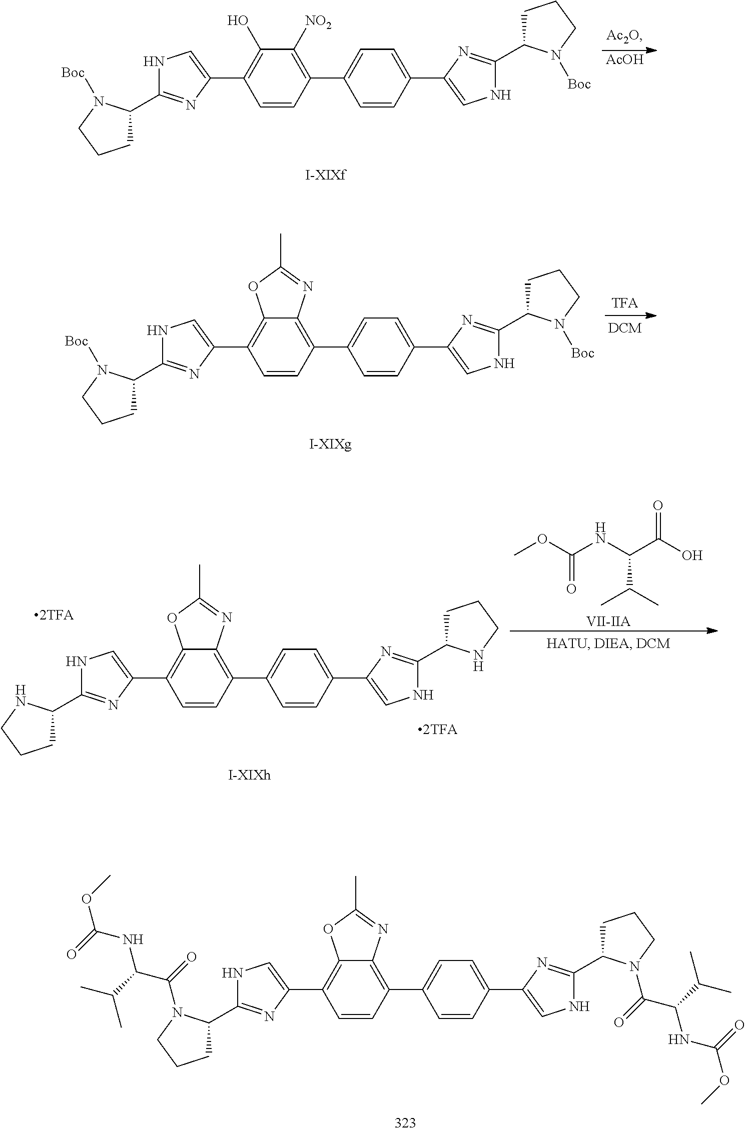

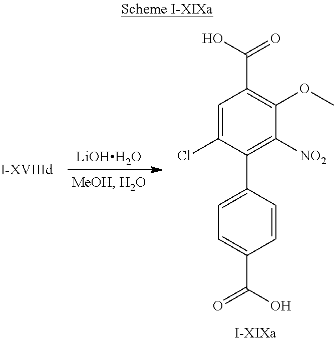

### General Procedure I-FR

Compound I-XVIIId (2.3 g, 6.07 mmol) and LiOH.H₂O (728 mg, 30.3 mmol) was added into 45 mL of THF/H₂O (2:1). The mixture was stirred overnight at room temperature. The mixture was acidified with aq. HCl (1 M), and extracted with EtOAc (100 mL×3), the organic layer was washed with water and brine, dried over anhydrous Na₂SO₄, and concentrated in vacuo to yield compound I-XIXa (2.2 g, yield 100%).

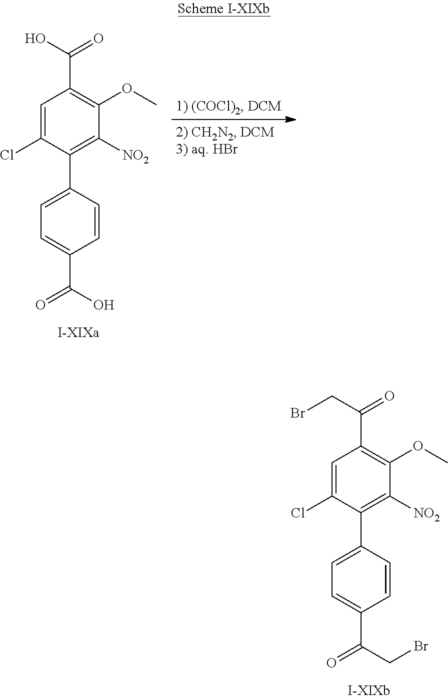

### General Procedure I-FS

Compound I-XIXa (850 mg, 2.42 mmol) was added into anhydrous DCM, and added (COCl)₂ in one portion (adding a drop of DMF as catalyst). The mixture was heated to reflux for 2 hrs. After that, the mixture was concentrated in vacuo to afford the acyl chloride, which was used in next step directly.

The acyl chloride was dissolved in 10 mL of DCM, and to the resulting solution was added a solution of diazomethane in ether (0.7 M, 40 mL, 28 mmol) at −5° C., the solution was stirred at r.t. for 2 hrs, 1 mL of aq. HBr (40%) was added into the solution at −5° C., and then the mixture was stirred overnight at room temperature. The reaction mixture was adjusted to pH=7 by addition of saturated aq. NaHCO₃, the organic layer was separated, dried over anhydrous Na₂SO₄ and concentrated under reduced pressure to afford compound I-XIXb (900 mg, yield 74%) as yellow solid.

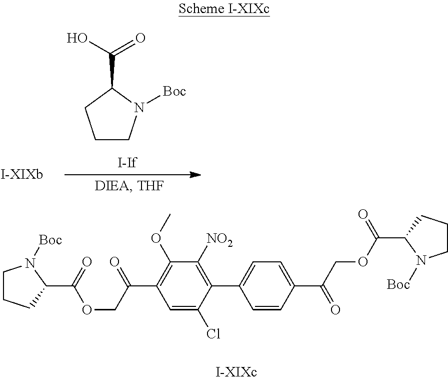

### General Procedure I-FT

Compound I-XIXb (2.3 g, 4.58 mmol), compound I-If (4.9 mg, 22.9 mmol) and DIEA (2.9 mg, 22.5 mmol) was added into 60 mL of THF. The mixture was stirred overnight at room temperature. TLC (PE:EtOAc=3:1) analysis showed disappearance of compound I-XIXb. The solution was quenched with water, extracted with EtOAc (100 mL×3), the organic layer was washed with water and brine, dried over anhydrous Na₂SO₄, and concentrated in vacuo, the residue was purified by column chromatography on silica gel to afford compound I-XIXc (2.1 g, yield 60%) as white solid.

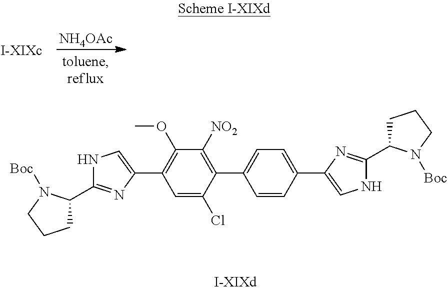

### General Procedure I-FU

Compound I-XIXc (2.1 g, 2.71 mmol) and NH₄OAc (4.18 mg, 54.3 mmol) was added into 50 mL of toluene. The mixture was heated to reflux overnight. After being cooled to r.t., water was added (100 mL), and the mixture was extracted with EtOAc (100 mL×3), the organic layer was washed with water and brine, dried over anhydrous Na₂SO₄, and concentrated in vacuo, the residue was purified by column chromatography on silica gel to provide compound I-XIXd (1.4 g, yield 70%) as white solid.

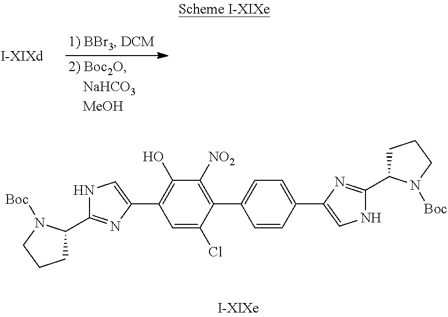

### General Procedure I-FV

BBr₃ (5.38 g, 21.6 mmol) was added into a solution of compound I-XIXd (1.58 g, 2.16 mmol) in 40 mL of anhydrous DCM at −60° C. to −70° C. The temperature was allowed to warm to room temperature and stirred overnight. The mixture was quenched by ice-water, and evaporated to remove solvent and then the mixture was diluted with MeOH (20 mL) and basified to pH=7˜8 with NaHCO₃. To the resulting mixture was added Boc₂O (1.04 g, 4.75 mmol) and NaHCO₃ (505 mg, 4.75 mmol), the reaction mixture was stirred for 3 hrs at room temperature. After the completion of reaction, the mixture was concentrated and added water, neutralized, extracted with EtOAc. The combined extracts was dried over anhydrous Na₂SO₄, and concentrated in vacuo to give compound I-XIXe (1.5 g, yield 96%).

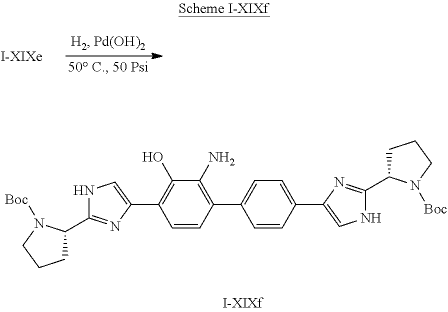

### General Procedure I-FW

To the mixture of Pd(OH)₂ (300 mg) in 100 mL of MeOH was added compound I-XIXe (2.4 g, 3.34 mmol). The mixture was stirred at 50° C. under hydrogen atmosphere (pressure 50 Psi) for 1 day. The solution was filtered and the solid was washed with MeOH. The filtrate was concentrated under reduced pressure to give compound I-XIXf (1.9 g, yield 87%).

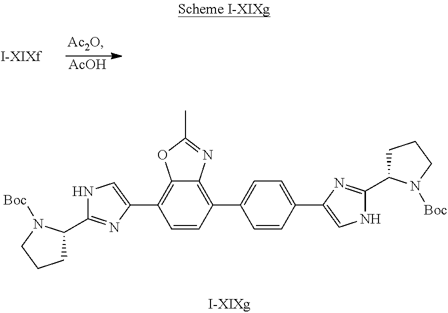

### General Procedure I-FX

A flask was charged with compound I-XIXf (311 mg, 0.475 mmol), AcOH (5 mL) and Ac₂O (72 mg, 0.712 mmol). The mixture was stirred at 100° C. for 1 h. After being cooled to r.t., the mixture was concentrated and added water, neutralized by saturated aq. NaHCO₃ solution, and extracted with EtOAc (50 mL×3), dried over anhydrous Na₂SO₄ and concentrated. The residue was purified by prep-TLC to provide compound I-XIXg (76 mg, yield 24%) as white solid.

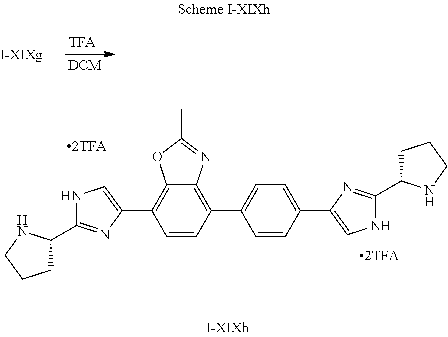

### General Procedure I-FY

To a solution of compound I-XIXg (86 mg, 0.126 mmol) in 4 mL of DCM was added TFA (2 mL). The mixture was stirred at r.t. for 3 hrs. While the reaction was completed, the solution was concentrated in vacuo to give compound I-XIXh, which was used for next step directly.

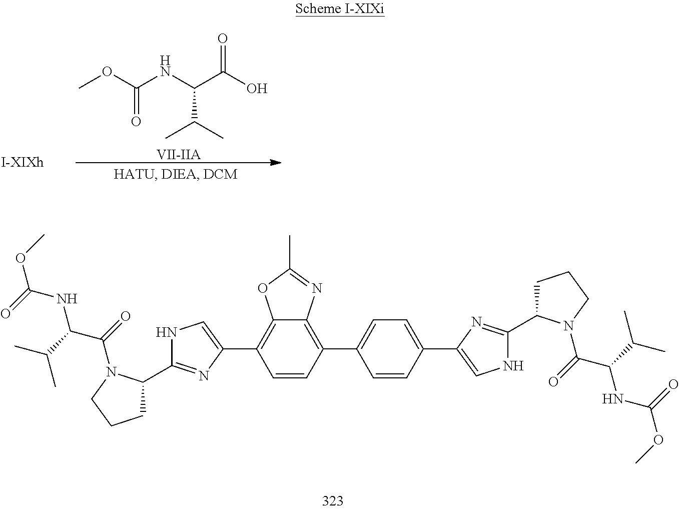

### General Procedure I-FZ

To a solution of compound I-XIXh (70 mg, 0.147 mmol) in 5 mL of CH₂Cl₂ was added DIEA (75.6 mg, 0.588 mmol), compound VII-IIA (51 mg, 0.294 mmol) and HATU (111 mg, 0.294 mmol). The reaction solution was stirred at r.t for 3 hrs. The mixture was diluted with CH₂Cl₂ (50 mL), washed with water and brine, the organic layer was dried over anhydrous Na₂SO₄, and concentrated in vacuo, the resulting residue was purified by Prep-HPLC to afford 323 (30 mg, yield 26%). MS (ESI) m/z [M+H]⁺ 794.5.

### Example I-XX

### Preparation of Compounds 324 and 325

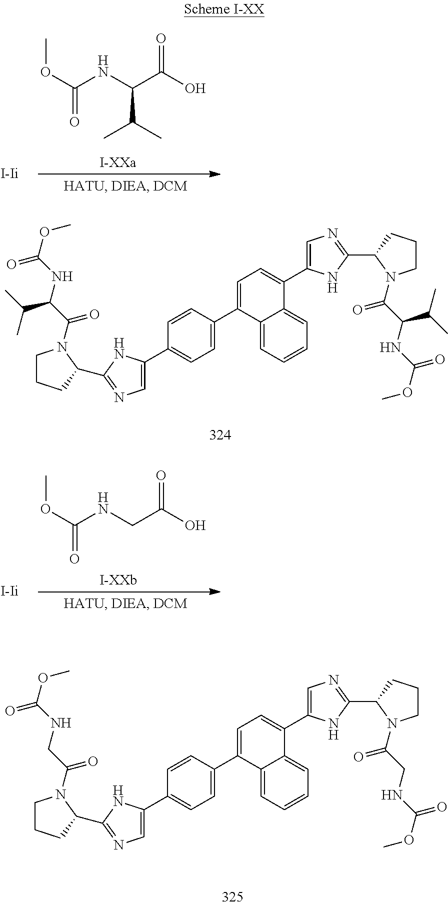

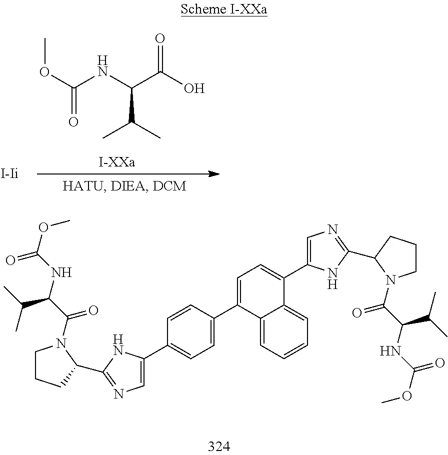

### General Procedure I-GA

To a solution of compound I-Ii (80 mg, 0.169 mmol) in anhydrous DCM (5 mL) were added compound I-XXa (59.2 mg, 0.338 mmol), HATU (128.4 mg, 0.338 mmol) and DIEA (54.4 mg, 0.42 mmol). The resulting mixture was stirred at r.t. overnight. After completion of the reaction, monitored by TLC, the mixture was poured into water (10 mL), extracted with CH₂Cl₂ (30 mL×3), the combined organic layers were dried over Na₂SO₄, concentrated in vacuo. The residue was purified by Prep-HPLC to afford compound 324 as a white solid (46 mg, yield 35%). MS (ESI) m/z (M+H)⁺ 789.4.

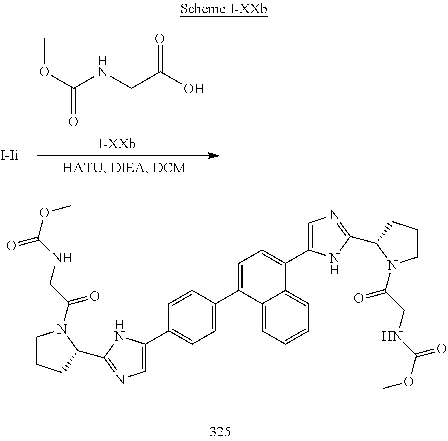

### General Procedure I-GB

To s solution of compound I-Ii (80 mg, 0.169 mmol) in anhydrous DCM (5 mL) were added N-methoxycarbonyl glycine (I-XXb; 45.1 mg, 0.338 mmol), HATU (128.4 mg, 0.338 mmol) and DIEA (54.4 mg, 0.42 mmol). The resulting mixture was stirred at r.t. overnight. After completion of the reaction, monitored by TLC, the reaction mixture was poured into water (10 mL), extracted with CH₂Cl₂ (30 mL×3), the combined organic layers were dried over Na₂SO₄, and concentrated in vacuo. The residue was purified by Prep-HPLC to afford compound 325 as a white solid (32 mg, yield 27%). MS (ESI) m/z (M+H)⁺ 705.3.

### Example I-XXI

### Preparation of Compounds 326

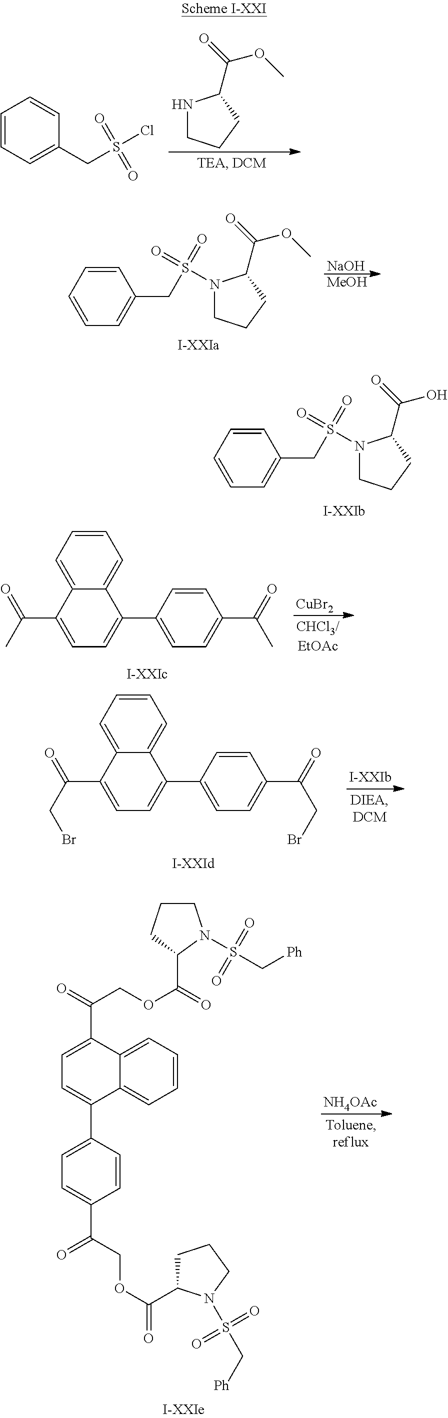

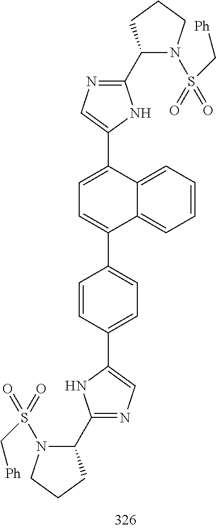

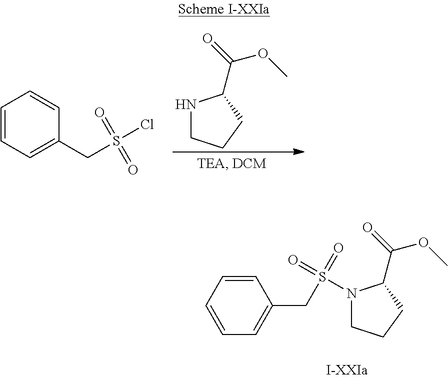

### General Procedure I-GC

L-Proline methyl ester (1 g, 5.2 mmol) and phenylmethanesulfonyl chloride (0.87 g, 5.2 mmol) were dissolved in DCM (10 mL), to the resulting solution was added TEA (1.58 g, 15.6 mmol) at 0° C., the reaction mixture was stirred at r.t. for 1 hour. Then the mixture was diluted with EtOAc (100 mL) and washed with water, dried over Na₂SO₄, concentrated in vacuo to afford compound I-XXIa (1.5 g, yield 100%), which was used in the next step without further purification.

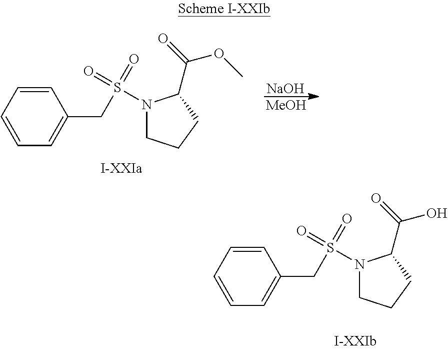

### General Procedure I-GD

To a solution of compound I-XXIa (0.8 g, 2.83 mmol) in MeOH (20 mL) was added NaOH (0.8 g, 20 mmol), the reaction mixture was stirred at 0° C. for 1 hour. Then the mixture was acidified with aq. HCl (1 M) to pH=4, and extracted with EtOAc (50 mL×3), washed with brine, dried over Na₂SO₄, and concentrated in vacuo to afford compound I-XXIb (0.7 g, yield 92%), which was used in the next step without further purification.

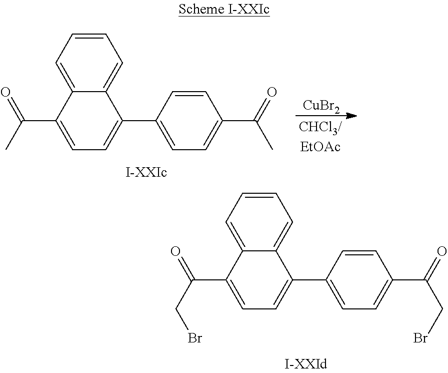

### General Procedure I-GE

To a solution of compound I-XXIc (0.3 g, 1.04 mmol) in chloroform (15 mL) and ethyl acetate (5 mL) was added CuBr₂ (573 mg, 2.6 mmol), the reaction mixture was refluxed for 3 hours. Then the mixture was cooled to r.t., diluted with EtOAc (100 mL), washed with brine, dried over Na₂SO₄, and concentrated in vacuo to afford compound I-XXId (240 mg, yield 44%), which was used in next step without further purification.

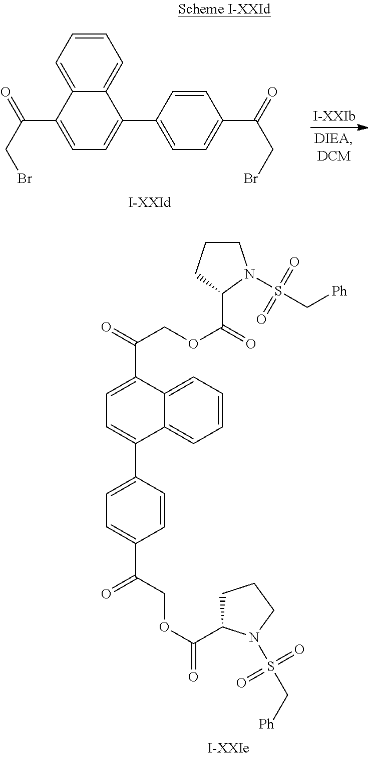

### General Procedure I-GF

To a solution of compound I-XXId (240 mg, 0.538 mmol) in DCM (20 mL) was added DIEA (206 mg, 1.6 mmol) and compound I-XXIb (288 mg, 1.03 mmol), the reaction mixture was stirred at r.t overnight. Then the mixture was diluted with EtOAc (100 mL), washed with brine, dried over Na₂SO₄, and concentrated in vacuo. The residue was purified with flash chromatography to afford compound I-XXIe (200 mg, yield 25%). MS (ESI) m/z (M+H)⁺ 823.1.

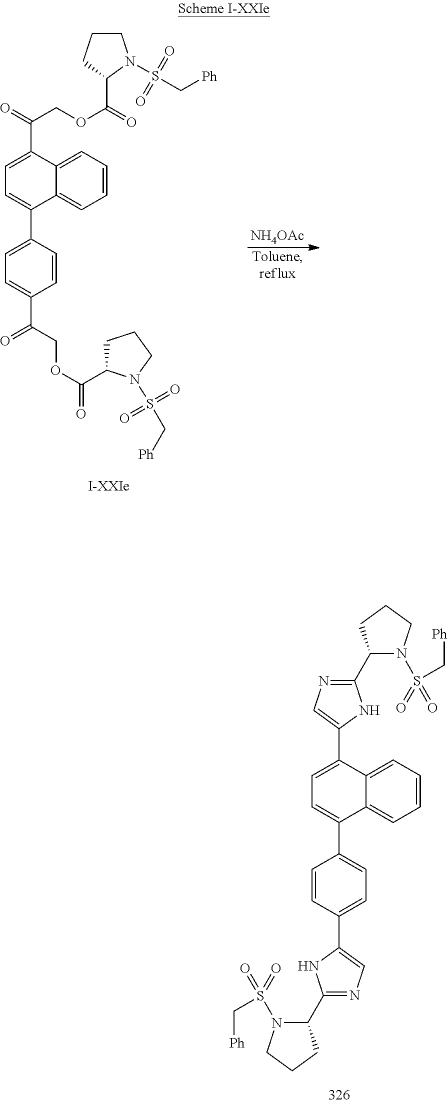

### General Procedure I-GG

To a mixture of compound I-XXIe (200 mg, 0.24 mmol) in toluene (5 mL) was added NH₄OAc (5 g, 65 mmol) and then the reaction mixture was heated to reflux overnight. Then the mixture was cooled to r.t., diluted with water (20 mL), extracted with EtOAc (30 mL×3), washed with brine, dried over Na₂SO₄, concentrated in vacuo. The residue was purified with prep-HPLC to provide compound 326 (29.3 mg, yield 15%). MS (ESI) m/z (M+H)⁺ 783.1.

### Example I-XXII

### Preparation of Compounds 327

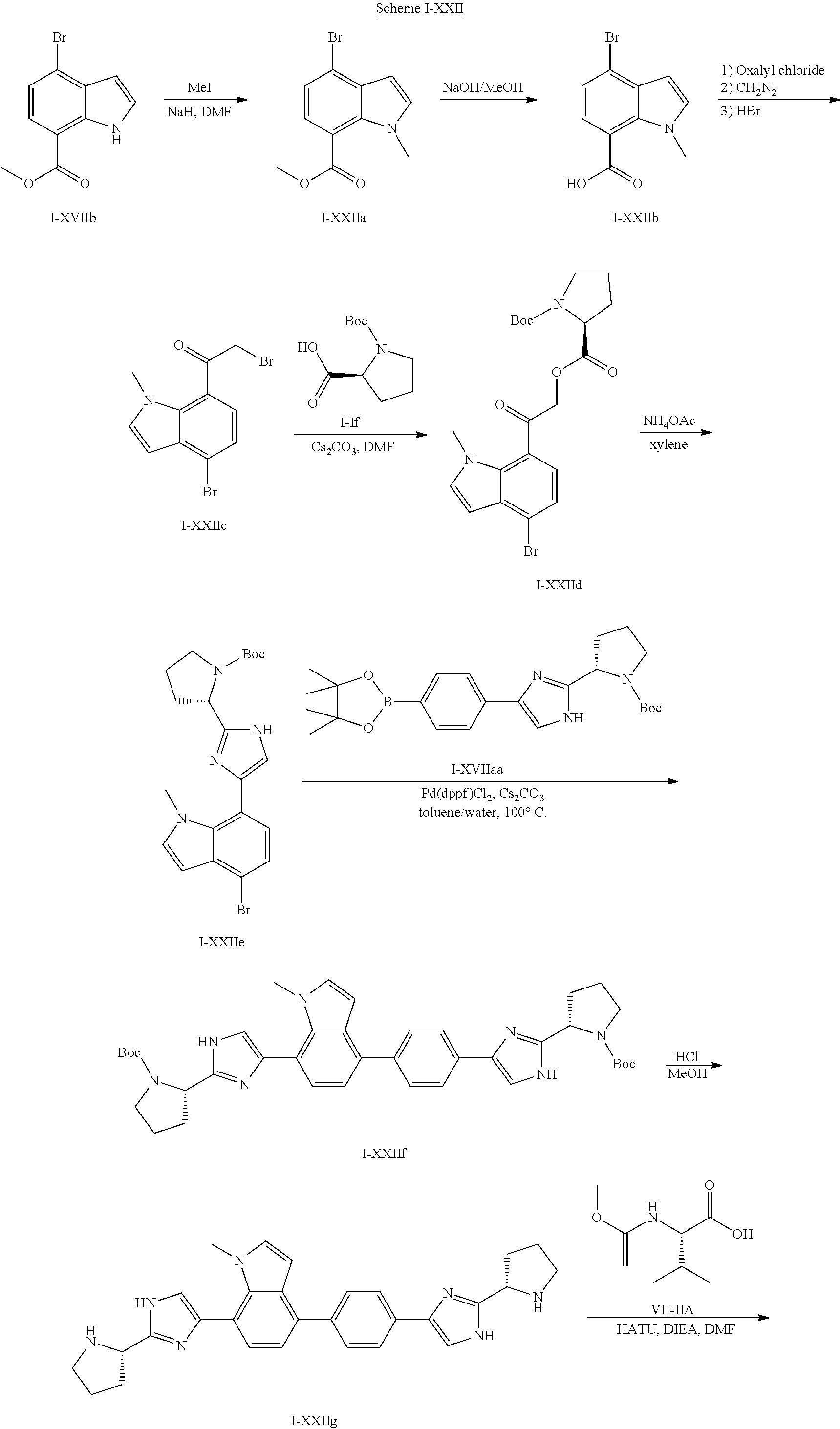

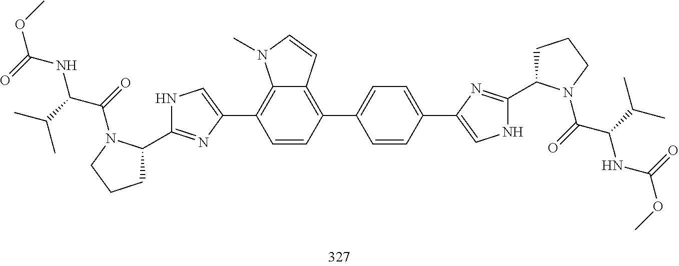

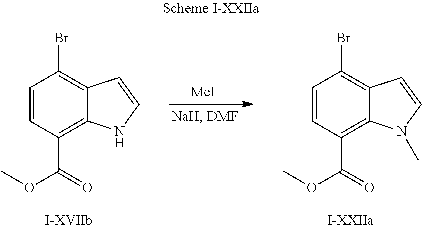

### General Procedure I-GH

To a mixture of compound I-XVIIb (1.0 g, 4.0 mmol) and NaH (60%, 0.32 g, 8.0 mmol) in 15 mL of dry DMF was added methyl iodide (MeI, 0.8 g, 6.0 mmol) dropwise at 0° C. under nitrogen, and the mixture was stirred at room temperature for 1 hour. The mixture was treated with water and extracted with EtOAc (30 mL). The organic phase was washed with water and brine, dried over anhydrous Na₂SO₄ and concentrated under reduced pressure. The residue was purified by column chromatography to afford compound I-XXIIa (0.6 g, yield 55%). ¹H NMR (400 MHz, CDCl₃): δ 7.56 (d, J=8.0 Hz, 1H), 7.29 (d, J=8.0 Hz, 1H), 7.12 (d, J=3.2 Hz, 1H), 6.62 (d, J=3.2 Hz, 1H), 3.96 (s, 3H), 3.89 (s, 3H).

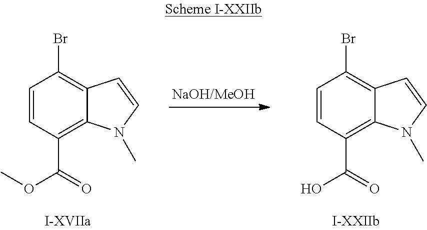

### General Procedure I-GI

A mixture of compound I-XXIIa (0.65 g, 2.4 mmol) and aq.NaOH (5 mL, 2N) in MeOH (5 mL) was stirred at 70° C. for 5 hours. After being cooled to room temperature, the mixture was acidified to pH 2-3 with 2N HCl and extracted with DCM (20 mL×3). The combined organic layer was washed with water and brine, dried over anhydrous Na₂SO₄ and concentrated to afford compound I-XXIIb (0.5 g, yield 81%). ¹H NMR (300 MHz, DMSO-d₆): δ 13.21 (s, 1H), 7.49-7.51 (m, 2H), 7.42 (d, J=8.1 Hz, 1H), 7.30 (d, J=7.8 Hz, 1H), 6.50 (d, J=3.0 Hz, 1H), 3.84 (s, 3H).

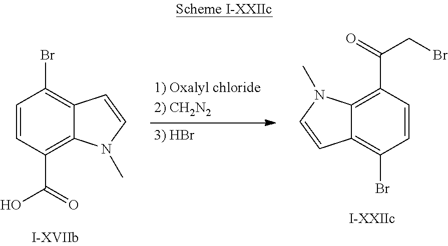

### General Procedure I-GJ

To a solution of compound I-XXIIb (0.5 g, 2.0 mmol) in dry DCM was added oxalyl chloride (0.4 g, 3.0 mmol) dropwise at 0° C. and the mixture was stirred at room temperature for 2 hours. After concentration, the residue was dissolved in dry DCM (10 mL), and the solution was added dropwise to another solution of diazomethane (8.0 mmol) in Et₂O (20 mL) at −10° C. under nitrogen protection. The mixture was stirred at room temperature for 2 hours. Then cooled again and aq. HBr (10 mL) was added dropwise and the mixture was stirred for another 1 hour. The reaction mixture was washed with aq. NaHCO₃ (30 mL) and brine, dried over anhydrous Na₂SO₄ and concentrated. The residue was purified by column chromatography to afford compound I-XXIIc (0.5 g, yield 75%). ¹H NMR (400 MHz, CDCl₃): δ 7.35-7.30 (m, 2H), 7.14 (d, J=3.2 Hz, 1H), 6.64 (d, J=3.2 Hz, 1H), 4.52 (s, 2H), 3.75 (s, 3H).

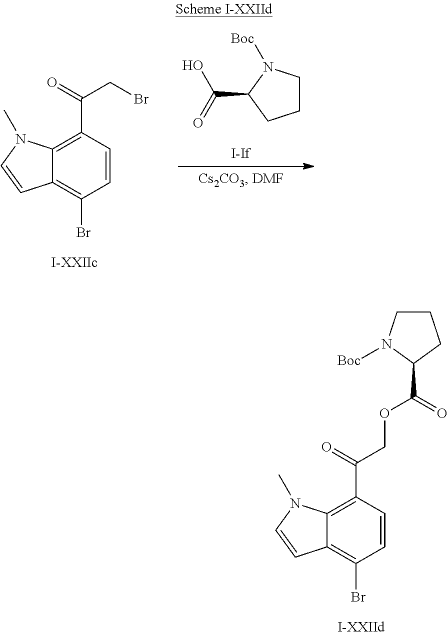

### General Procedure I-GK

The mixture of compound I-XXIIc (500 mg, 1.5 mmol), compound I-If (390 mg, 1.8 mmol) and Cs₂CO₃ (1.0 g, 3 mmol) in DMF (10 mL) was stirred at room temperature for 2 hours. The reaction mixture was then diluted with EtOAc (30 mL), and the resulting mixture was washed with water and brine, dried over anhydrous Na₂SO₄ and concentrated. The residue was purified by column chromatography to afford compound I-XXIId (500 mg, yield 71%).

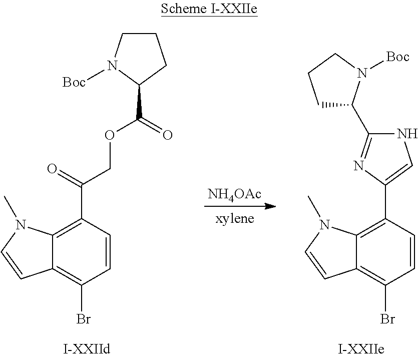

### General Procedure I-GL

A mixture of compound I-XXIId (400 mg, 0.86 mmol) and NH₄OAc (1.3 g, 17.2 mmol) in xylene (15 mL) was stirred at 180° C. for 5 hours in a sealed tube. After cooling to r.t., the mixture was diluted with EtOAc (20 mL), and the resulting mixture was washed with water and brine, dried over anhydrous Na₂SO₄ and concentrated under reduced pressure. The residue was purified by column chromatography to afford compound I-XXIIe (100 mg, yield 26%).

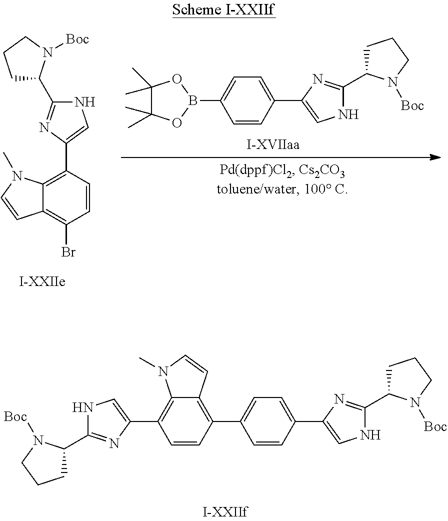

### General Procedure I-GM

A mixture of compound I-XXIIe (80 mg, 0.18 mmol), compound I-XVIIaa (95 mg, 0.22 mmol), Pd(dppf)Cl₂ (10% mol) and Cs₂CO₃ (117 mg, 0.36 mmol) in 6 mL of toluene/water (5/1) was stirred at 100° C. for 3 hours. After cooling to r.t., the mixture was diluted with EtOAc (20 mL), washed with brine, dried over anhydrous Na₂SO₄ and concentrated. The residue was purified by column chromatography to afford compound I-XXIIf (100 mg, yield 82%).

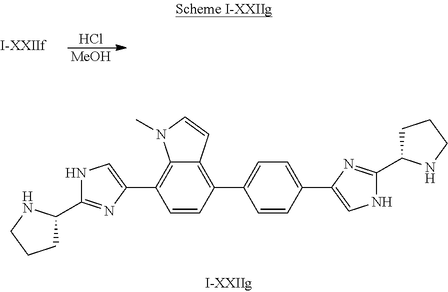

### General Procedure I-GN

To a solution of compound I-XXIIf (100 mg, 0.15 mmol) in methanol (5 mL) was added 5 mL of HCl/MeOH and the mixture was stirred at 60° C. for 3 hours. Concentration under reduced pressure afford compound I-XXIIg as a residue that was used to the next step without further purification.

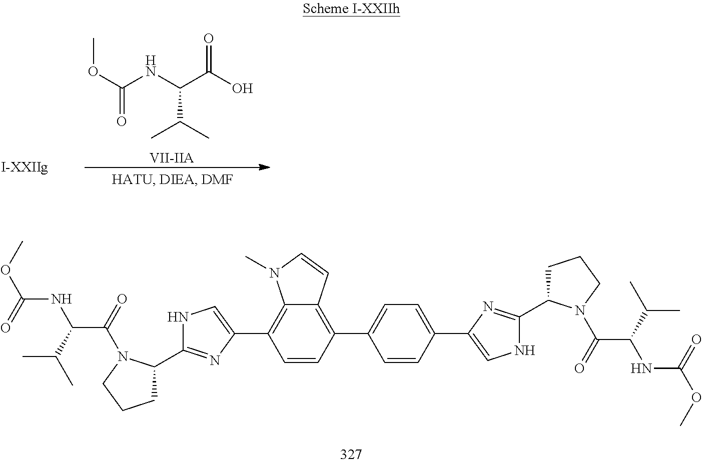

### General Procedure I-GO

HATU (116 mg, 0.30 mmol) was added to a mixture of compound VII-IIA (66 mg, 0.38 mmol), compound I-XXIIg (70 mg, 0.15 mmol), and DIEA (58 mg, 0.45 mmol) in DMF (5 mL), the resulting mixture was stirred at room temperature for 2 hours. After being diluted with EtOAc (20 mL), the organic layer was washed with brine, dried over Na₂SO₄ and concentrated. The residue was purified by prep-HPLC to afford compound 327 (20 mg, yield 17%). ¹H NMR: (400 MHz, CD₃OD): δ 7.67-7.82 (m, 4H), 7.11-7.34 (m, 5H), 6.62 (s, 1H), 5.17-5.34 (m, 2H), 4.21-4.24 (m, 2H), 3.83-3.99 (m, 4H), 3.50-3.72 (m, 9H), 2.21-2.37 (m, 5H); 2.01-2.06 (m, 5H), 0.88-1.00 (m, 12H). MS (ESI) m/z (M+H)⁺ 792.4.

### Example I-XXIII

### Preparation of Compounds 328

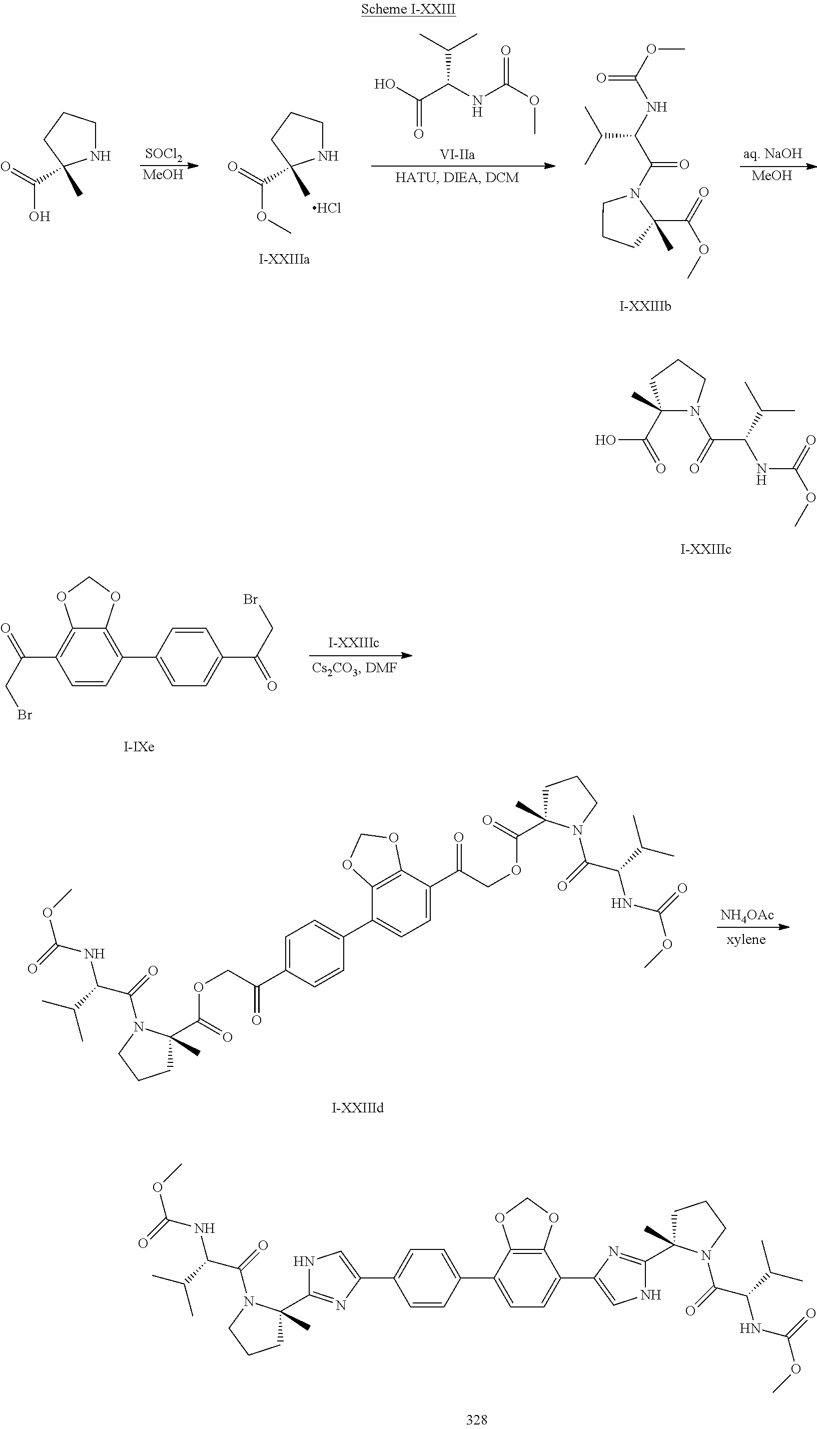

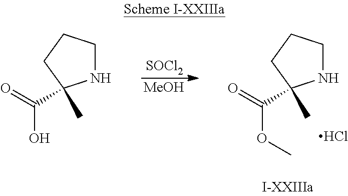

### General Procedure I-GP

To a mixture of 2-methyl-L-proline (1.0 g, 7.8 mmol) in 20 mL of dry methanol was added SOCl₂ (2.8 g, 23.3 mmol) dropwise at 0° C. under nitrogen protection. The resulting mixture was stirred at room temperature overnight, and then the solvent was removed under reduced pressure to afford compound I-XXIIIa as an HCl salt (1.4 g, yield 100%). ¹H NMR (300 MHz, CD₃OD): δ 3.86 (s, 3H), 3.42-3.46 (m, 2H), 2.36-2.45 (m, 1H), 2.00-2.19 (m, 3H), 1.68 (s, 3H).

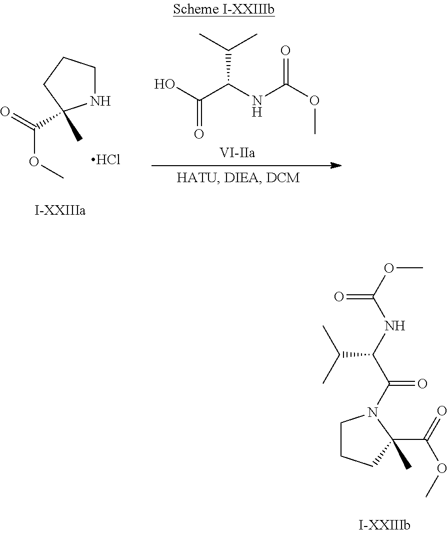

### General Procedure I-GQ

To a solution of compound I-XXIIIa (1.35 g, 7.7 mmol) in 30 mL of DCM was added compound VI-IIa (1.5 g, 8.5 mmol), HATU (4.4 g, 11.6 mmol) and DIEA (3 g, 23 mmol). The resulting mixture was stirred at room temperature overnight. Subsequently, the mixture was diluted with DCM and washed with brine. The organic layers were dried over anhydrous Na₂SO₄ and concentrated. The residue was purified by column chromatography (PE/EA=3/1) to afford compound I-XXIIIb (1.5 g, yield 65%). MS (ESI) m/z (M+H)⁺ 301.

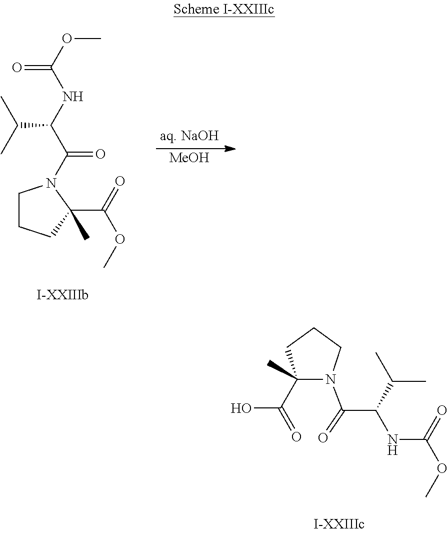

### General Procedure I-GR

A mixture of compound I-XXIIIb (1.5 g, 5 mmol) and NaOH (0.6 g, 15 mmol) in MeOH (30 mL) and H₂O (5 mL) was stirred at 70° C. for 2 hours. The methanol under reduce pressure and the residue was dissolved with 20 mL of H₂O, then the solution was acidified to pH 2-3 with 2N HCl and extracted with DCM (50 mL×2). The organic layer was washed with brine, dried over anhydrous Na₂SO₄ and concentrated to afford compound I-XXIIIc (0.8 g, yield 57%), which was used in next step without further purification. ¹H NMR (300 MHz, DMSO-d₆): δ 12.20 (s, 1H), 7.24 (d, J=8.4 Hz, 1H), 3.54-3.98 (m, 3H), 3.50 (s, 3H), 1.78-2.05 (m, 5H), 1.34 (s, 3H), 0.84-0.88 (m, 6H).

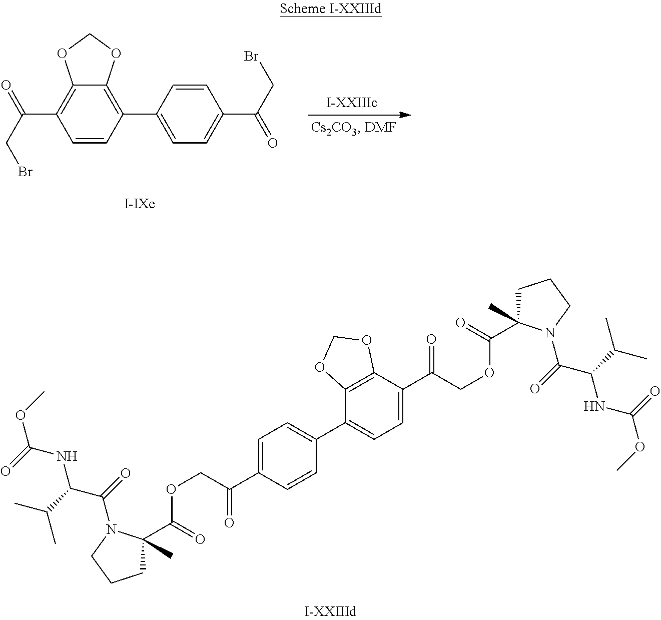

### General Procedure I-GS

A mixture of compound I-IXe (100 mg, 0.23 mmol), compound I-XXIIIc (162 mg, 0.57 mmol) and Cs₂CO₃ (150 mg, 0.46 mmol) in DMF (5 mL) was stirred at room temperature for 2 hours. Then the mixture was diluted with EtOAc (30 mL), washed with brine. The organic layer was separated, dried over anhydrous Na₂SO₄ and concentrated. The residue was purified by Prep-TLC (DCM/MeOH=20/1) to afford compound I-XXIIId (100 mg, yield 52%). MS (ESI) m/z (M+H)⁺ 851.

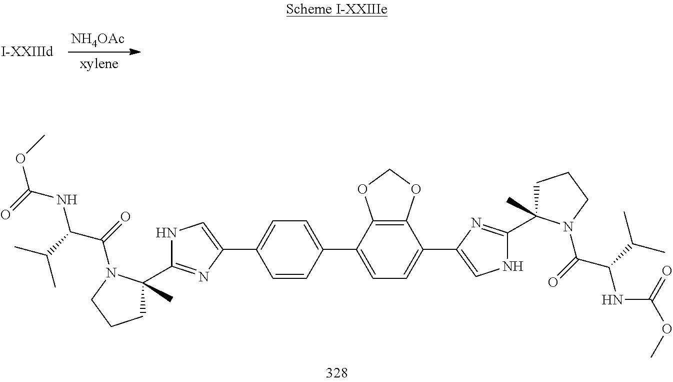

### General Procedure I-GT

A mixture of compound I-XXIIId (100 mg, 0.12 mmol) and NH₄OAc (185 mg, 2.4 mmol) in 10 mL of xylene was stirred at 120° C. for 5 hours in a sealed tube. After cooling to r.t., the solvent was removed under reduce pressure and the residue was diluted with EtOAc (30 mL), and washed with brine. The organic layer was separated, dried over anhydrous Na₂SO₄ and concentrated. The residue was purified by Prep-HPLC to afford compound 328 (20 mg, yield 21%). ¹H NMR (300 MHz, CD₃OD): δ 7.70-7.79 (m, 4H), 7.15-7.44 (m, 4H), 6.13 (s, 2H), 4.18 (d, J=6.9 Hz, 2H), 3.87-4.08 (m, 4H), 3.67 (s, 6H), 2.48-2.55 (m, 2H), 1.99-2.14 (m, 8H), 1.86 (s, 6H), 0.86-0.97 (m, 12H). MS (ESI) m/z (M+H)⁺ 811.5.

### Example I-XXIV

### Preparation of Compounds 329

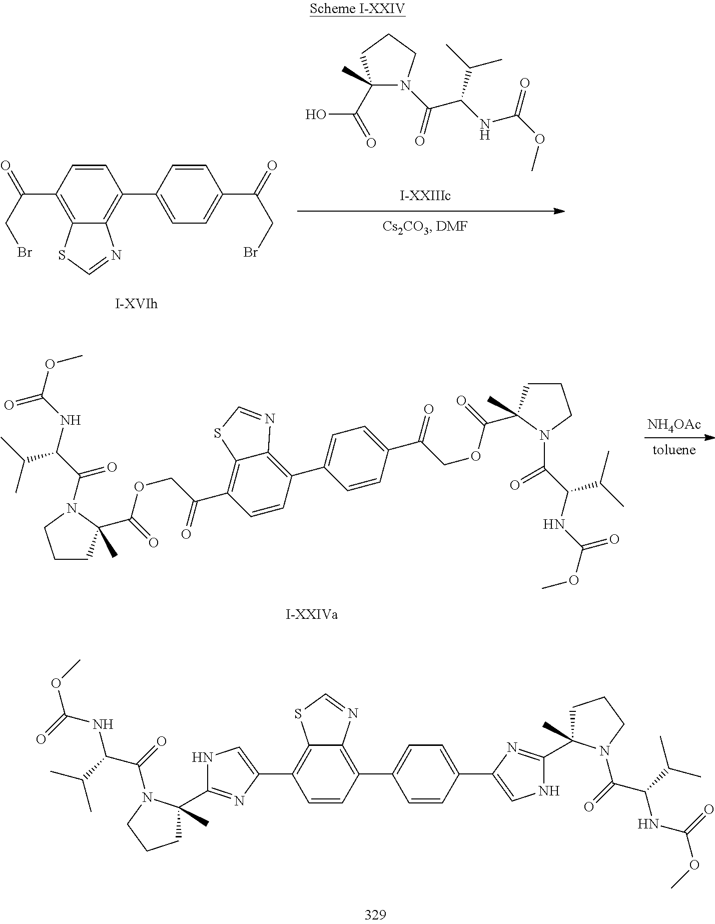

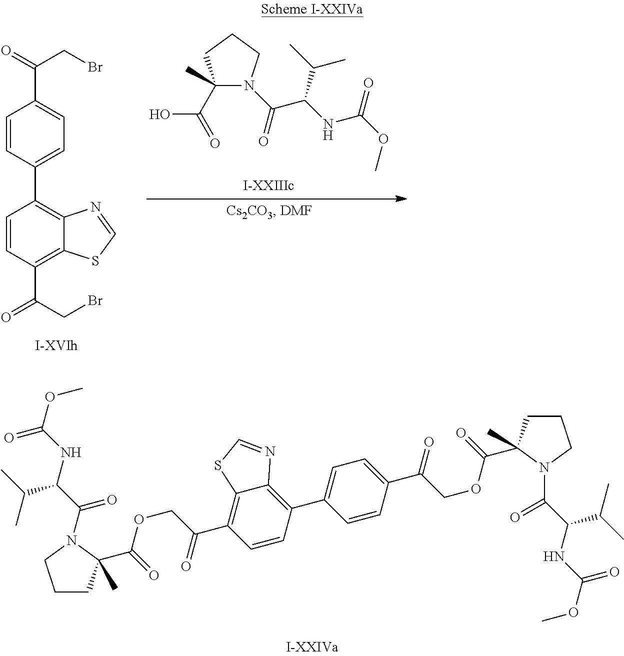

### General Procedure I-GU

Compound I-XVIh (160 mg, 0.36 mmol) was dissolved DMF (5 mL). To the resulting solution was added compound I-XXIIIc (233 mg, 0.82 mmol) and Cs₂CO₃ (267 mg, 0.82 mmol). The reaction mixture was stirred for 2 hrs at room temperature. Then the mixture was diluted with water (20 mL) and neutralized with diluted HCl (1 N), extracted with EtOAc (20 mL×3). The combined organic layers were washed with brine, dried over anhydrous Na₂SO₄ and concentrated. The residue was purified by Prep-TLC to afford compound I-XXIVa (50 mg, yield 16%).

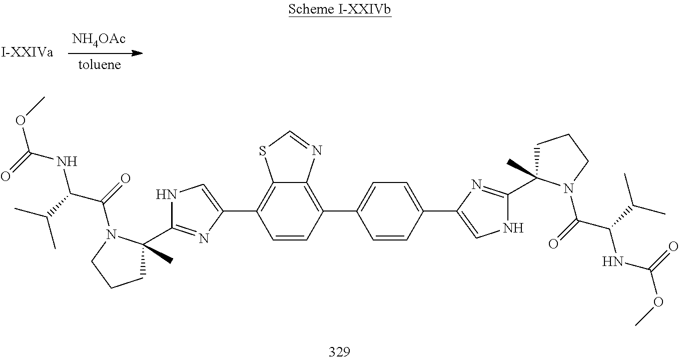

### General Procedure I-GV

Compound I-XXIVa (50 mg, 0.06 mmol) and NH₄OAc (2 g, 25.9 mmol) in 5 mL of toluene was heated at 160° C. in a sealed tube. After 3 hours, the mixture was cooled to r.t, diluted with water (40 mL) and extracted with EtOAc (20 mL×3). The combined organic layers were dried over anhydrous Na₂SO₄ and concentrated. The residue was purified by Prep-HPLC to afford compound 329 (5 mg, yield 11%). ¹H NMR (400 MHz, CD₃OD): δ 9.22 (s, 1H), 7.90 (d, J=7.6 Hz, 1H), 7.88-7.77 (m, 4H), 7.63 (d, J=7.6 Hz, 1H), 7.57 (s, 1H), 7.33 (s, 1H), 4.23-4.19 (m, 2H), 4.10-4.02 (m, 2H), 4.00-3.90 (m, 2H), 3.66 (s, 6H), 2.72-2.66 (m, 1H), 2.61-2.52 (m, 1H), 2.38-2.32 (m, 1H), 2.20-1.98 (m, 7H), 1.92 (s, 3H), 1.88 (s, 3H), 1.00-0.82 (m, 12H). MS (ESI) m/z (M+H)⁺ 824.2.

### Example I-XXV

### Preparation of Compound 330

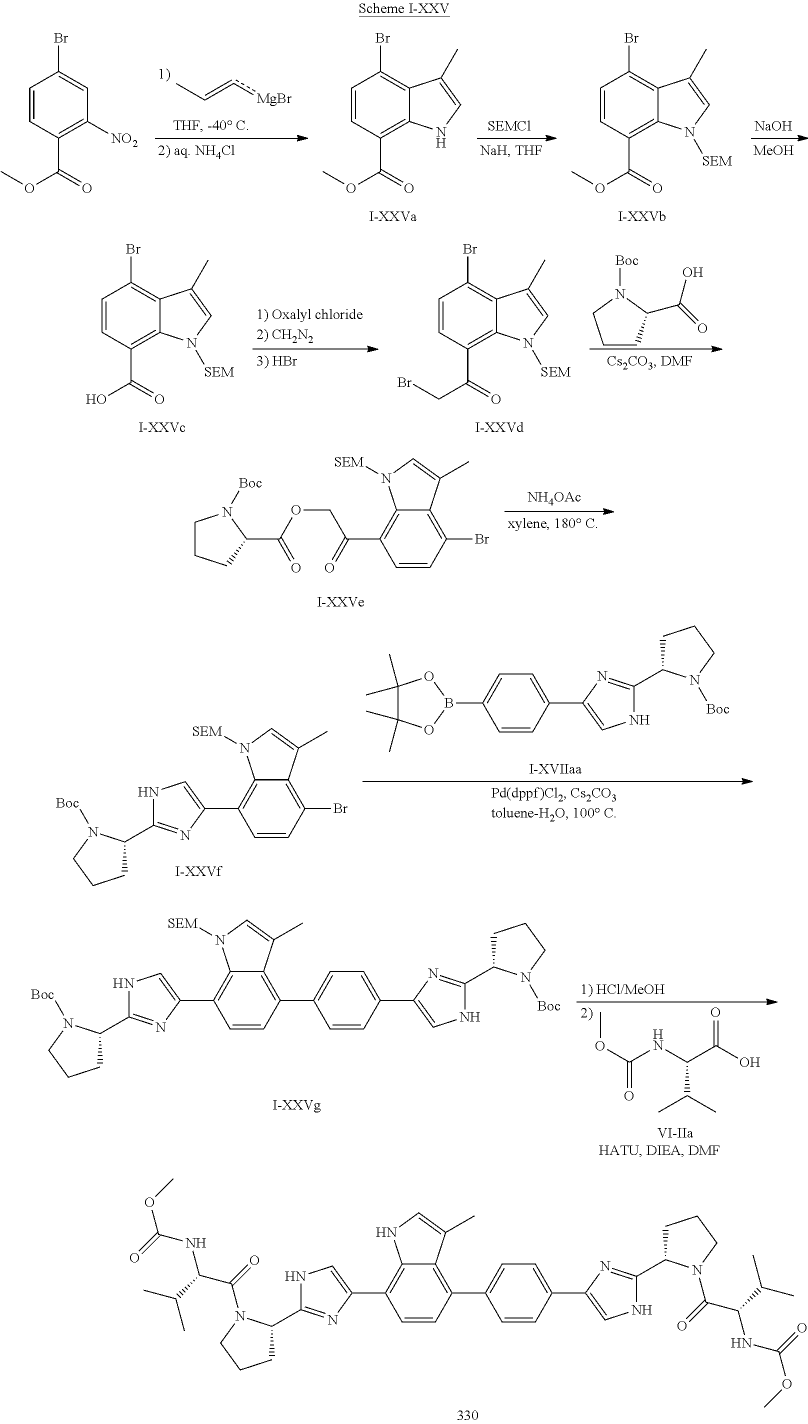

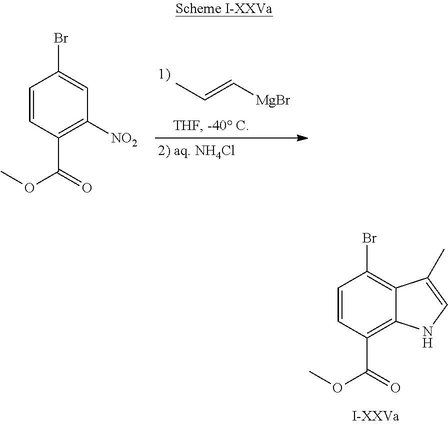

### General Procedure I-GW

To a solution of methyl 4-bromo-2-nitrobenzoate (5.2 g, 20 mmol) in 20 mL of dry THF was added (E)-prop-1-en-1-yl magnesium bromide (100 mL, 50 mmol) dropwise at −40° C. under nitrogen protection. The mixture was stirred at room temperature for 5 hours. Subsequently, the mixture was treated with aq. NH₄Cl, and then extracted with EtOAc (50 mL×2). The organic layer was washed with water and brine, dried over anhydrous Na₂SO₄ and concentrated under reduced pressure. The residue was purified by column chromatography to afford compound I-XXVa (1.1 g, yield 20%). ¹H NMR (400 MHz, CDCl₃): δ 9.74 (s, 1H), 7.65 (d, J=8.0 Hz, 1H), 7.27 (d, J=8.0 Hz, 1H), 7.10 (s, 1H), 3.96 (s, 3H), 2.56 (s, 3H).

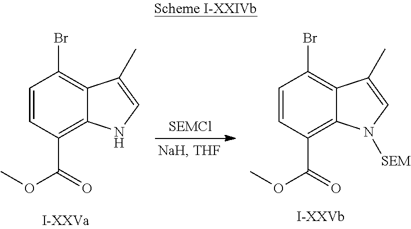

### General Procedure I-GX

To a mixture of compound I-XXVa (1.0 g, 3.7 mmol) and NaH (0.3 g, 7.4 mmol) in 10 mL of dry THF was added 2-trimethylsilylethoxymethyl chloride (0.9 g, 5.6 mmol) dropwise at 0° C. under nitrogen. The mixture was stirred at room temperature for 1 hour. The mixture was treated with water, extracted with EtOAc (30 mL×3). The organic layer was washed with water and brine, dried over anhydrous Na₂SO₄ and concentrated. The residue was purified by column chromatography to afford compound I-XXVb (1.0 g, yield 67%). ¹H NMR (400 MHz, CDCl₃): δ 7.48 (d, J=8.0 Hz, 1H), 7.38 (d, J=8.0 Hz, 1H), 7.07 (s, 1H), 5.67 (s, 2H), 4.03 (s, 3H), 3.24 (t, J=8.0 Hz, 2H), 2.64 (s, 3H), 0.84 (t, J=8.0 Hz, 2 H), 0.00 (s, 9H).

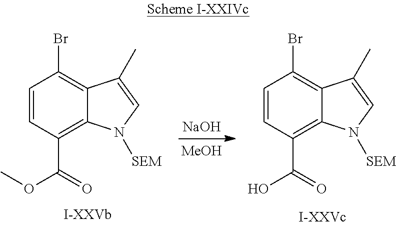

### General Procedure I-GY

A mixture of compound I-XXVb (1.1 g, 2.8 mmol) and NaOH (5 mL, 2N) in MeOH (5 mL) was stirred at 70° C. for 2 hours. After cooling to r.t., the mixture was acidified to pH 2˜3 with aq. HCl (2 M) and extracted with DCM (20 mL×3). The organic layer was dried over Na₂SO₄ and concentrated to afford compound I-XXVc (1.0 g, yield 91%). ¹H NMR (300 MHz, DMSO-d6): δ 13.08 (s, 1H), 7.44-7.57 (m, 3H), 5.79 (s, 2H), 3.27 (t, J=7.8 Hz, 2H), 2.62 (s, 3H), 0.82 (t, J=8.1 Hz, 2H), 0.00 (s, 9H).

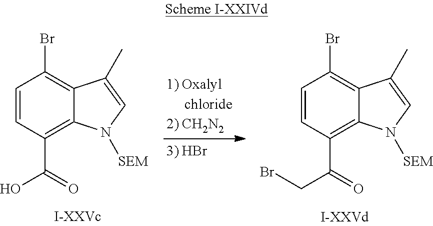

### General Procedure I-GZ

To a solution of compound I-XXVc (1.0 g, 2.6 mmol) in 10 mL of dry DCM was added oxalyl chloride (0.5 g, 3.9 mmol) at 0° C. and the mixture was stirred at room temperature for 2 hours. The solvent was removed under reduced pressure and the residue was re-dissolved in 10 mL of dry DCM. This solution was added dropwise to a mixture of diazomethane (7.8 mmol) in 40 mL of Et₂O at −10° C. under nitrogen. The resulting mixture was stirred at room temperature for 2 hours and then cooled to −10° C. Subsequently, 10 mL of aq. HBr (48%) was added dropwise and the mixture was stirred for an additional 1 hour. The resulting mixture was washed with saturated aq. NaHCO₃ and brine, dried over anhydrous Na₂SO₄, and concentrated. The residue was purified by column chromatography (PE/EA=10/1) to afford compound I-XXVd (330 mg, yield 28%). ¹H NMR (400 MHz, CDCl₃): δ 7.39 (d, J=8.0 Hz, 1H), 7.28 (d, J=8.0 Hz, 1H), 7.06 (s, 1H), 5.40 (s, 2H), 4.64 (s, 2H), 3.24 (t, J=8.4 Hz, 2H), 2.61 (s, 3H), 0.84 (t, J=8.4 Hz, 2H), 0.00 (s, 9H).

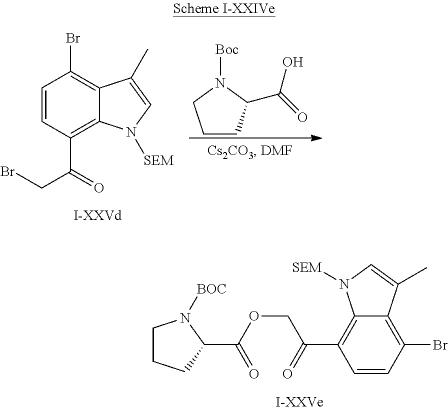

### General Procedure I-HA

A mixture of compound I-XXVd (330 mg, 0.72 mmol), N-Boc-proline (128 mg, 0.60 mmol) and Cs₂CO₃ (470 mg, 1.2 mmol) in DMF (10 mL) was stirred at room temperature for 2 hours. Subsequently, the mixture was diluted with EtOAc (50 mL), washed with brine, dried over anhydrous Na₂SO₄, and concentrated. The residue was purified by column chromatography (PE/EA=3/1) to afford compound I-XXVe (350 mg, yield 83%). MS (ESI) m/z (M+H)⁺ 597.

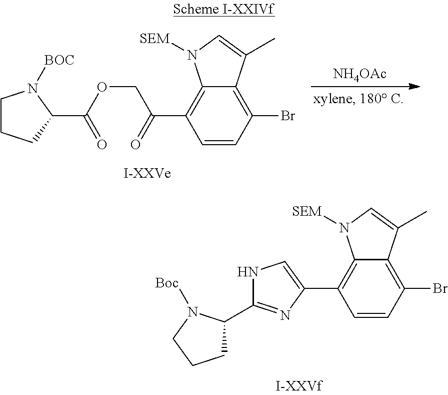

### General Procedure I-HB

A mixture of compound I-XXVe (350 mg, 0.6 mmol) and NH₄OAc (900 mg, 12 mmol) in xylene (15 mL) was stirred at 180° C. for 5 hours in a sealed tube. After cooling to r.t., the mixture was diluted with EtOAc (20 mL), washed with brine (100 mL), dried over anhydrous Na₂SO₄, and concentrated. The residue was purified by Prep-TLC (PE/EA=1/1) to afford compound I-XXVf (100 mg yield 30%). MS (ESI) m/z (M+H)⁺ 577.

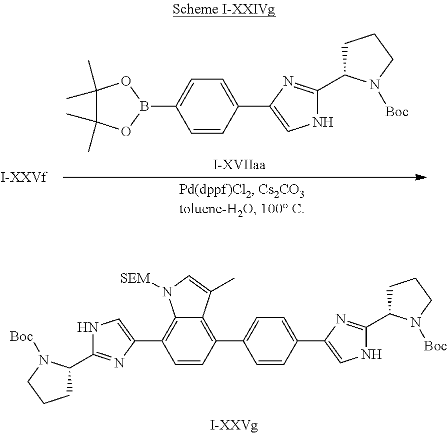

### General Procedure I-HC

A flask was charged with compound I-XXVf (100 mg, 0.17 mmol), compound I-XVIIaa (84 mg, 0.19 mmol), Pd(dppf)Cl₂ (10% mol) and Cs₂CO₃ (111 mg, 0.34 mmol) in 5 mL of toluene/water (v/v=5/1), the mixture was stirred at 100° C. for 2 hours. After cooling to r.t., the mixture was diluted with EtOAc (30 mL), washed with brine, dried over anhydrous Na₂SO₄ and concentrated. The residue was purified by Prep-TLC (DCM/MeOH=20/1) to afford compound I-XXVg (100 mg, yield 71%). MS (ESI) m/z (M+H)⁺ 808.

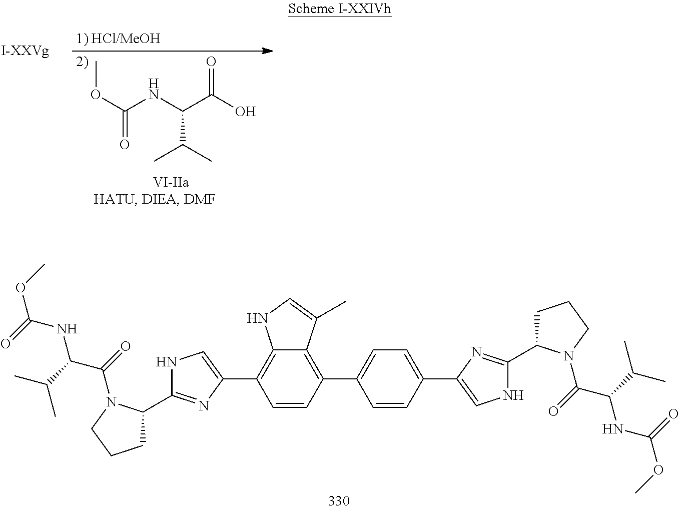

### General Procedure I-HD

To a solution of compound I-XXVg (100 mg, 0.12 mmol) in methanol (5 mL) was added a solution of HCl/MeOH (4 M, 5 mL). The resulting mixture was stirred at 70° C. for 2 hours. After removal the solvent, the residue was dissolved in DMF (5 mL), and then compound VI-IIa (44 mg, 0.25 mmol), HATU (91 mg, 0.24 mmol) and DIPEA (52 mg, 0.4 mmol) were added. The mixture was stirred at room temperature for 2 hours and then the mixture was diluted with EtOAc (40 mL), washed with brine, dried over anhydrous Na₂SO₄ and concentrated under reduced pressure. The residue was purified by prep-HPLC to afford compound 330 (20 mg, yield 21%). ¹H NMR (300 MHz, CD₃OD): δ 7.68-7.77 (m, 2H), 7.32-7.42 (m, 5H), 7.07 (s, 1H), 6.86-6.88 (m, 1H), 5.16-5.37 (m, 2H), 4.21-4.25 (m, 2H), 3.62-3.99 (m, 4H), 3.50 (s, 6H), 2.19-2.33 (m, 5H), 2.02-2.06 (m, 5H), 1.90 (s, 3H), 0.89-1.02 (m, 12H). MS (ESI) m/z (M+H)⁺ 792.4.

### Example I-XXVI

### Preparation of Compound 331

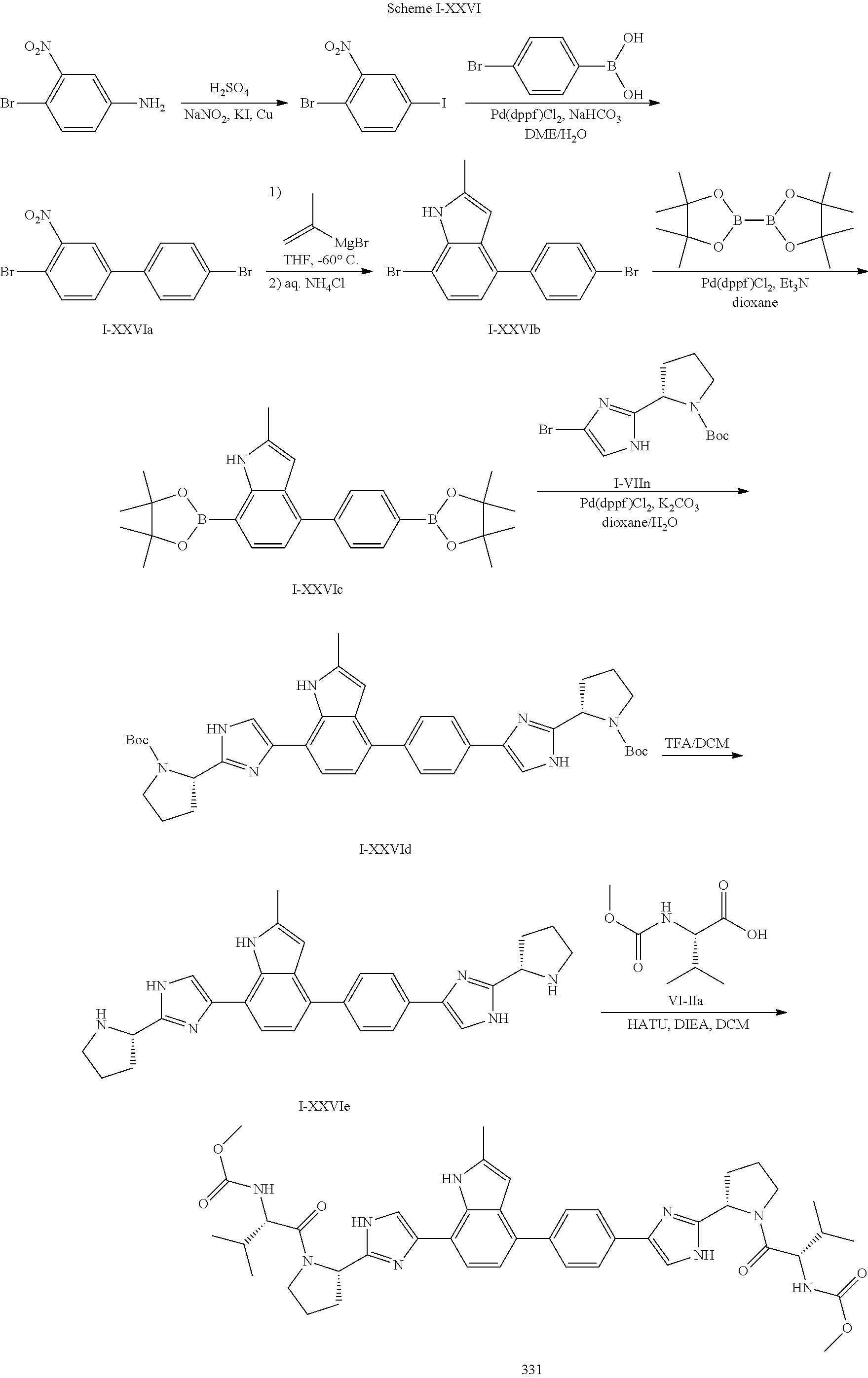

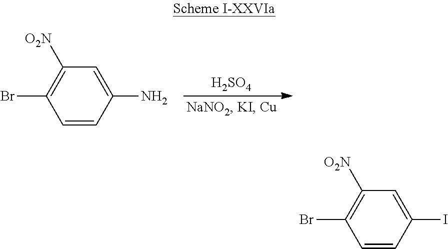

### General Procedure I-HE

To a solution of 4-bromo-3-nitroaniline (10.0 g, 46 mmol) in H₂O (150 mL) was added H₂SO₄ (10 mL). The mixture was stirred at room temperature for 30 minutes and then cooled to 0° C. A mixture of NaNO₂ (3.3 g, 48 mmol) in H₂O (50 mL) was added slowly at 0° C. and the resulting mixture was stirred at same temperature for 3 hours. Subsequently, a solution of KI (10 g, 60 mmol) in H₂O (50 mL) was added, after a few minutes, Cu (0.01 g, 1 mmol) was added. The resulting mixture was stirred at room temperature overnight. The mixture was extracted with EtOAc (200 mL×2), the combined organic lawyers were washed with sat. Na₂S₂O₃, dried over Na₂SO₄ and concentrated. The crude product was purified by column chromatography (PE/EA=10/1) to afford 1-bromo-4-iodo-2-nitrobenzene (7.0 g, yield 47%).

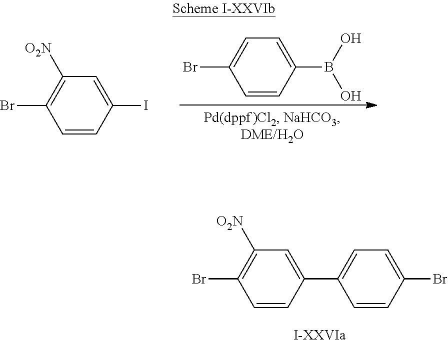

### General Procedure I-HF

To a mixture of 1-bromo-4-iodo-2-nitrobenzene (3.5 g, 10.7 mmol), 4-bromophenylboronic acid (2.6 g, 13.1 mmol) and NaHCO₃ (1.8 g, 21.4 mmol) in DME/H₂O (30 mL/10 mL) was added Pd(dppf)Cl₂ (0.35 g). The resulting mixture was stirred at 80° C. for 5 hours. After cooling to r.t., the mixture was diluted with water (60 mL) and extracted with EtOAc (150 mL×3). The combined organic layer was dried over Na₂SO₄ and concentrated. The crude product was purified by column chromatography (PE) to afford compound I-XXVIa (1.5 g, yield 39%).

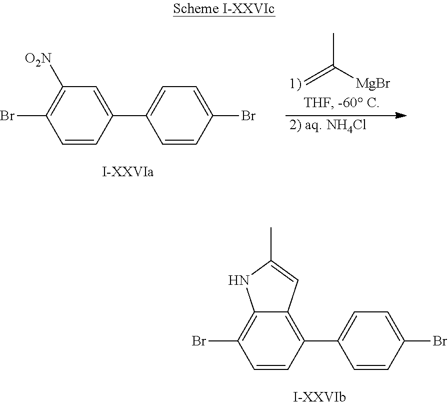

### General Procedure I-HG

To a solution of compound I-XXVIa (2.2 g, 6.2 mmol) in anhydrous THF (20 mL) was added prop-1-en-2-yl magnesium bromide (37 mL, 18.5 mmol) at −45° C. and the mixture was stirred at the same temperature for 1 hour. The mixture was quenched with aq.NH₄Cl (30 mL) and then extracted with EtOAc (100 mL×3). The organic layers were separated, dried over Na₂SO₄ and concentrated. The residue was purified by column chromatography (PE) to afford compound I-XXVIb (0.9 g, yield 45%). MS (ESI) m/z (M+H)⁺ 364.

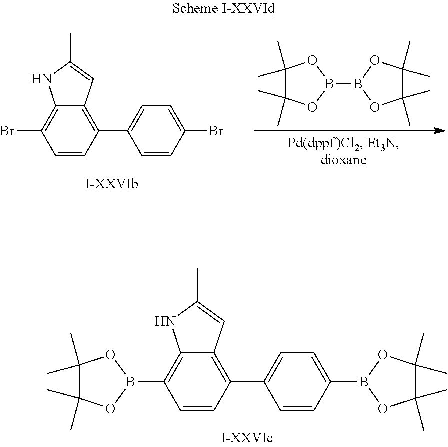

### General Procedure I-HH

A mixture of compound I-XXVIb (0.4 g, 0.9 mmol), bis(pinacolato)diboron (0.7 g, 2.7 mmol), Et₃N (0.65 g, 6.4 mmol), Pd(dppf)Cl₂ (0.04 g) in dioxane (4 mL) was irradiated in microwave at 150° C. for 30 minutes. The mixture was cooled to r.t., diluted with water (10 mL) and extracted with EtOAc (50 mL×2). The organic layers were separated, dried over Na₂SO₄ and concentrated. The crude product was purified by Prep-TLC (PE/EA=10/1) to afford compound I-XXVIc (0.3 g, yield 60%). MS (ESI) m/z (M+H)⁺ 460.

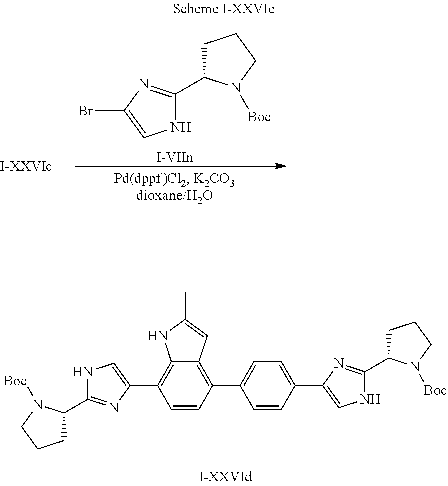

### General Procedure I-HI

To a mixture of compound I-XXVIc (0.3 g, 1.1 mmol), compound I-VIIn (0.55 g, 1.7 mmol) and K₂CO₃ (0.50 g, 3.6 mmol) in dioxane/H₂O (3 mL/0.5 mL) was added Pd(dppf)Cl₂ (0.03 g). The reaction mixture was stirred at reflux overnight under nitrogen protection. After cooling to r.t., the mixture was diluted with H₂O (10 mL) and extracted with EtOAc (50 mL×2). The organic layers were dried over Na₂SO₄ and concentrated. The residue was purified by Prep-TLC (EA/MeOH=20/1) to afford compound I-XXVId (0.03 g, yield 7%). MS (ESI) m/z (M+H)⁺ 678.

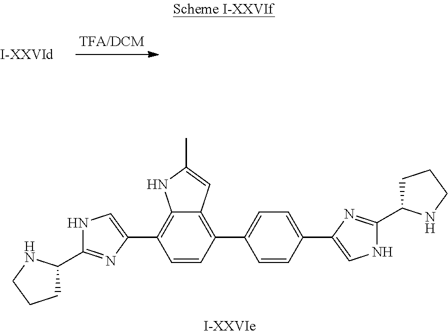

### General Procedure I-HJ

TFA (2 mL) was added to a solution of compound I-XXVId (0.05 g, 0.07 mmol) in DCM (2 mL), the mixture was stirred at room temperature for 1 hour. The mixture was concentrated under reduced pressure to afford compound I-XXVIe as a TFA salt, which was used in the next step without further purification.

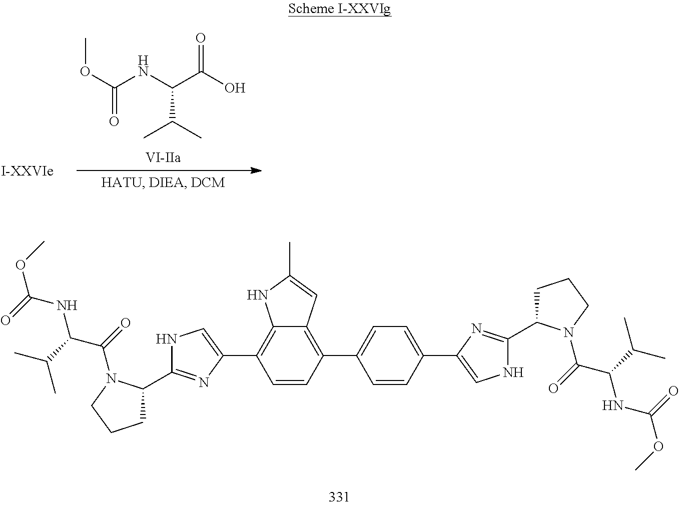

### General Procedure I-HK

To a mixture of compound I-XXVIe (35 mg, 0.07 mmol), compound VI-IIa (26 mg, 0.15 mmol), DMA (30 mg, 0.23 mmol) in DCM (2 mL) was added HATU (62 mg, 0.16 mmol). The resulting mixture was stirred at room temperature for 1 hour. Subsequently, water (10 mL) was added and the mixture was extracted with EtOAc (50 mL×2). The combined organic layer was separated, dried over Na₂SO₄ and concentrated. The residue was purified by Prep-HPLC to afford compound 331 (13 mg, yield 22%). ¹H NMR (400 MHz, CDCl₃): δ 10.68-10.77 (m, 2H), 7.84 (d, J=8.0 Hz, 1H), 7.71 (d, J=8.4 Hz, 2H), 7.49 (d, J=8.0 Hz, 1H), 7.34-7.37 (m, 2H), 7.23-7.26 (m, 1H), 7.09-7.15 (m, 1H), 6.41-6.45 (m, 1H), 5.26-5.50 (m, 2H), 4.31-4.35 (m, 2H), 3.73-3.89 (m, 2H), 3.72 (s, 6H), 3.62-3.63 (m, 2H), 2.98-3.20 (m, 2H), 2.53 (s, 3H), 1.98-2.41 (m, 10H), 0.85-0.89 (m, 12H). MS (ESI) m/z (M+H)⁺ 792.5.

### Section II

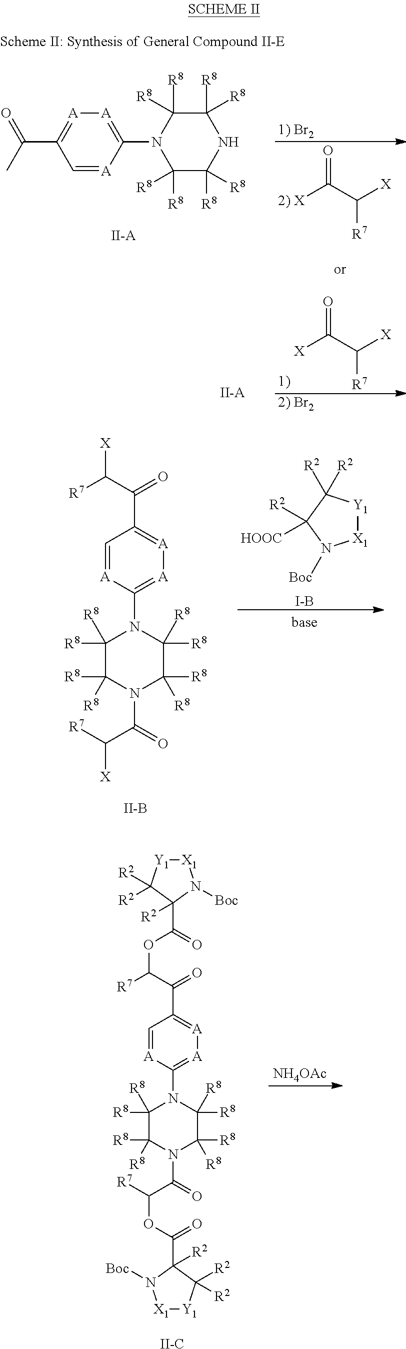

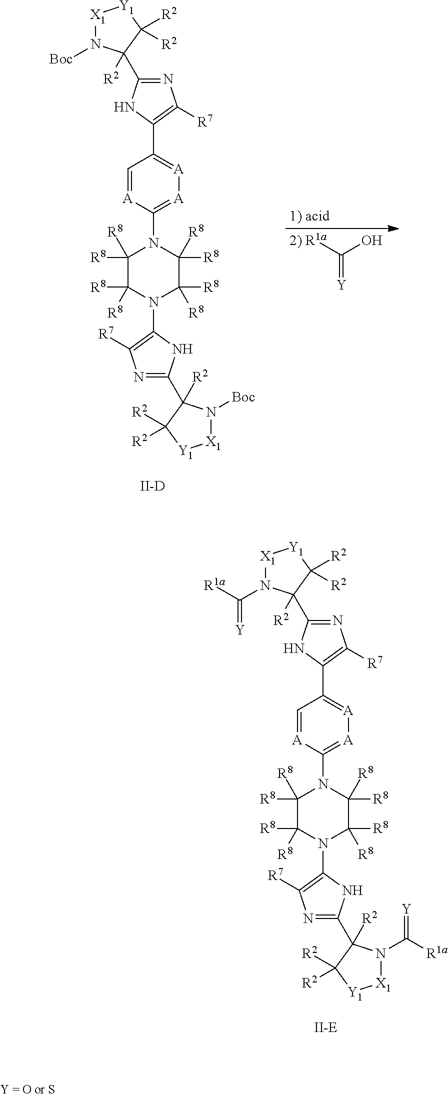

In some embodiments, the acyl halide in the step converting II-A to II-B has the structure

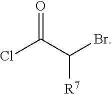

In some embodiments, the base in the step converting II-B to II-C is DIEA in THF. In some embodiments, the step converting II-C to II-D is conducted in toluene. In some embodiments, the acid used in the step converting II-D to II-E is HCl in methanol. In some embodiments, the carboxylic acid used in the step converting II-D to II-E is

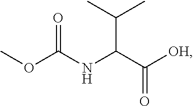

which may be formed according to the following reaction:

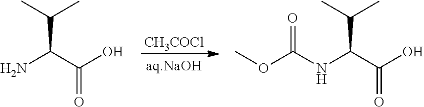

The compounds shown below in Table II can be prepared by the methods disclosed in Scheme II, modified as appropriate. It will be readily apparent to one of ordinary skill in the art that the compounds shown below in Table II can be synthesized by use of the appropriate reactants, reagents and reaction conditions.

TABLE II

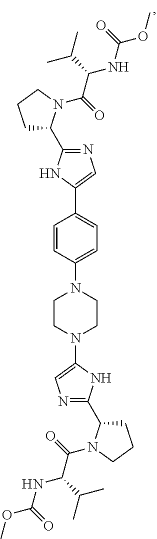

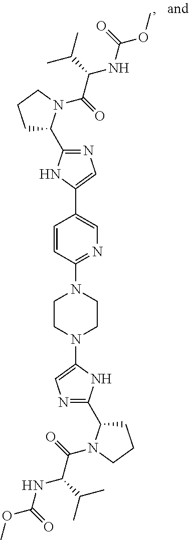

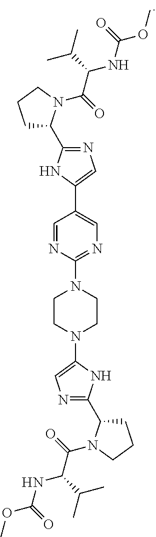

### Section IIII

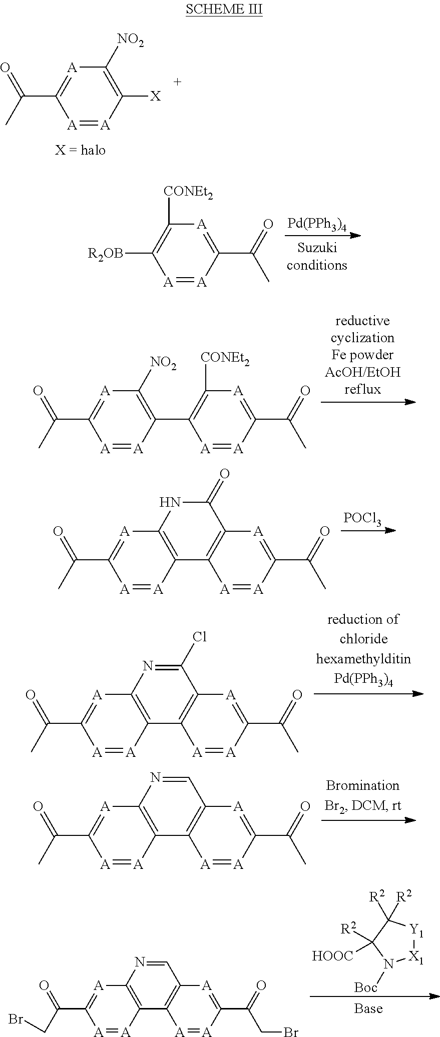

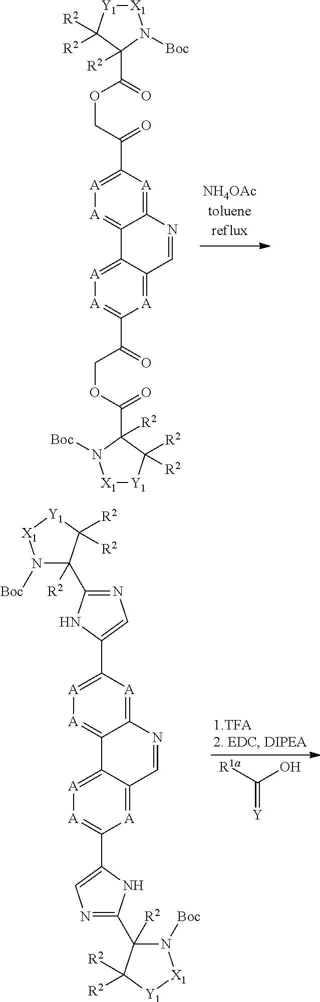

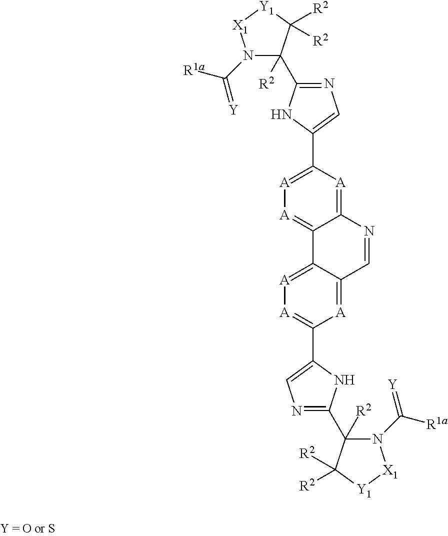

The compounds shown below in Table III can be prepared by the methods disclosed in Scheme III, modified as appropriate. It will be readily apparent to one of ordinary skill in the art that the compounds shown below in Table III can be synthesized by use of the appropriate reactants, reagents and reaction conditions.

TABLE III

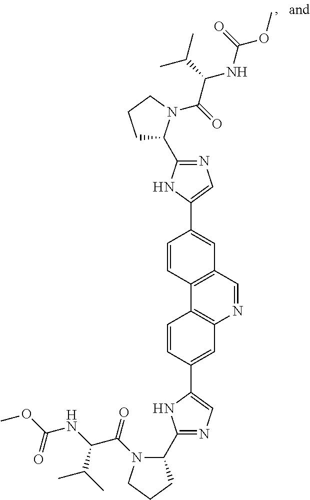

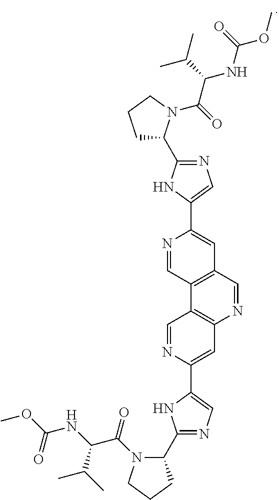

### Section IV

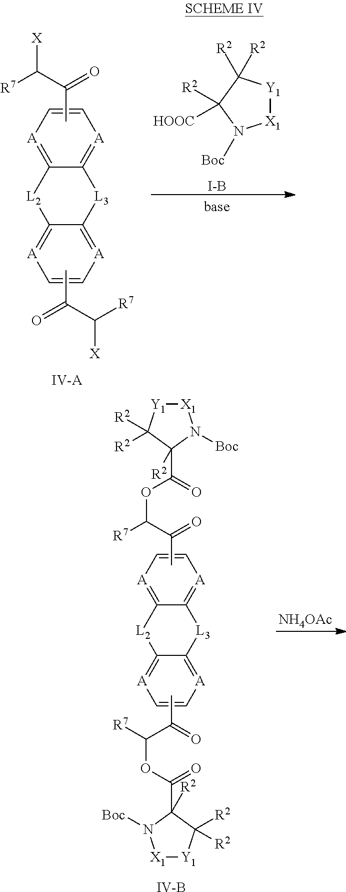

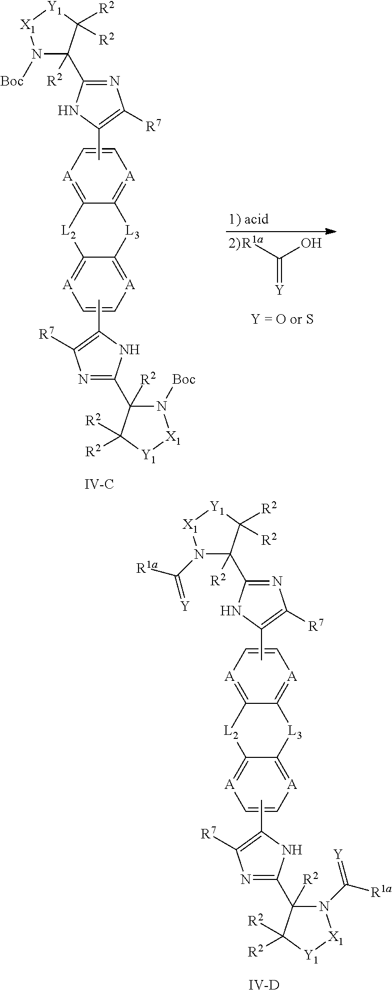

The compounds shown below in Table IV can be prepared by the methods disclosed in Scheme IV, modified as appropriate. It will be readily apparent to one of ordinary skill in the art that the compounds shown below in Table IV can be synthesized by use of the appropriate reactants, reagents and reaction conditions.

TABLE IV

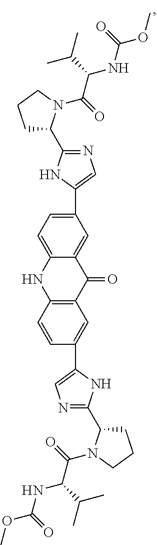

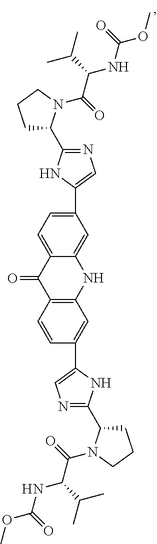

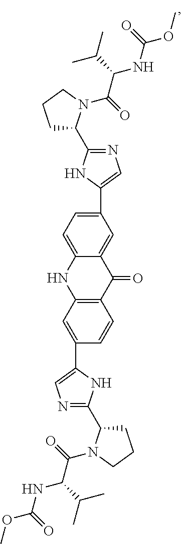

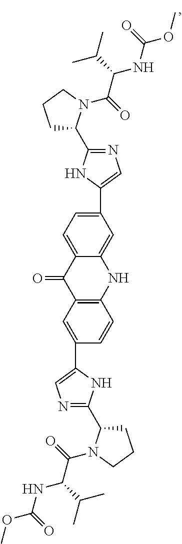

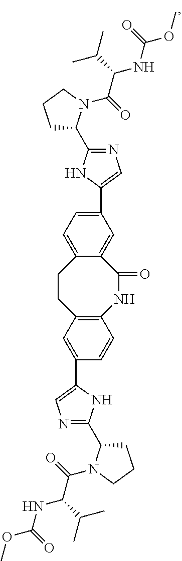

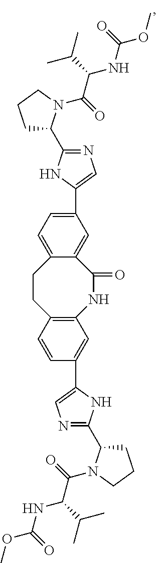

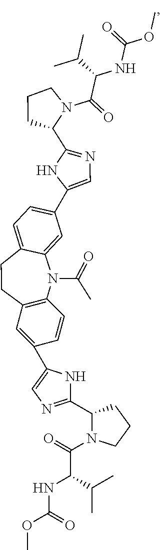

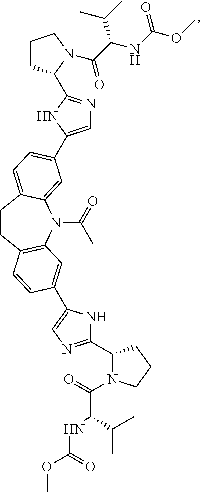

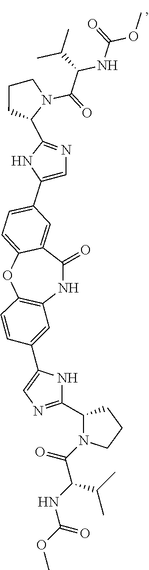

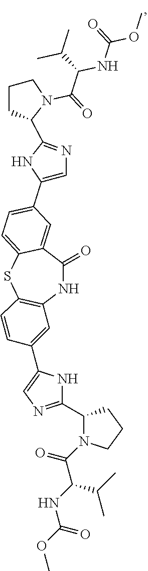

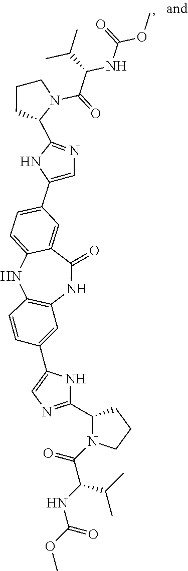

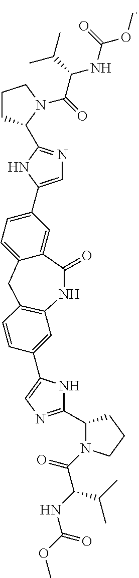

### Section V

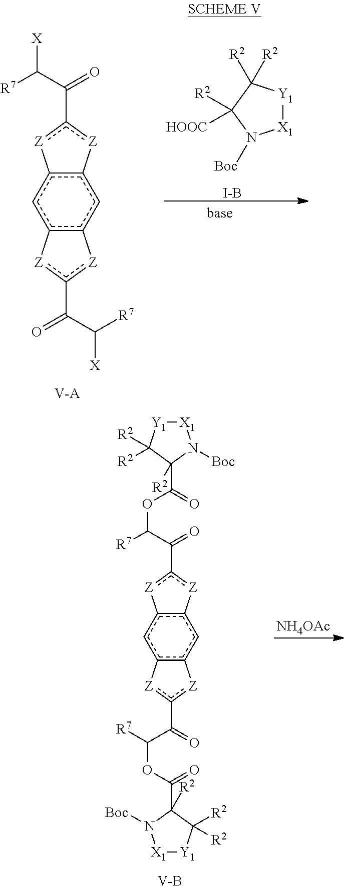

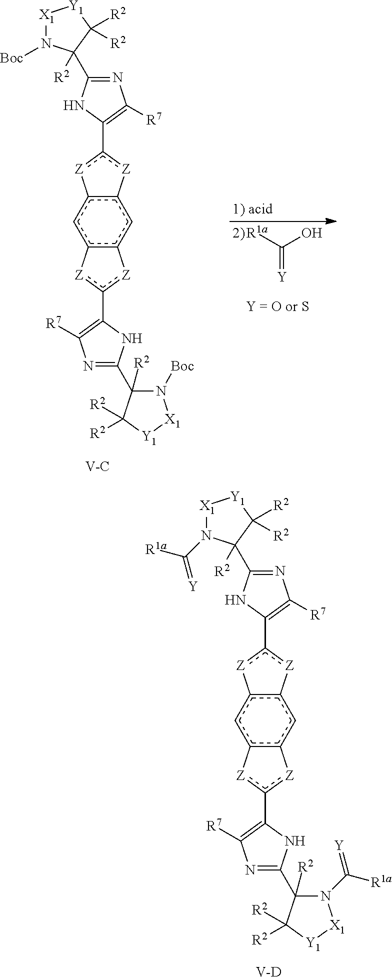

The compounds shown below in Table V can be prepared by the methods disclosed in Scheme V, modified as appropriate. It will be readily apparent to one of ordinary skill in the art that the compounds shown below in Table V can be synthesized by use of the appropriate reactants, reagents and reaction conditions.

TABLE V

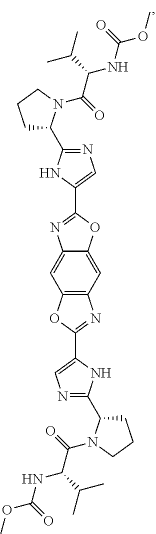

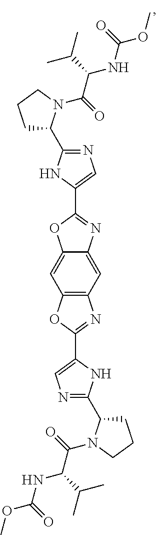

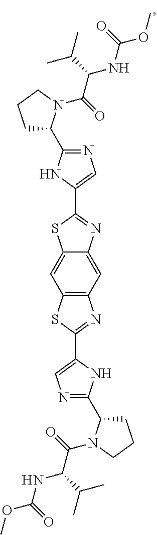

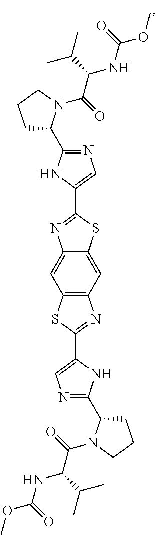

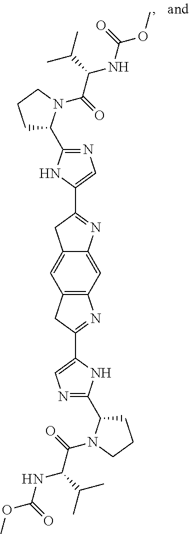

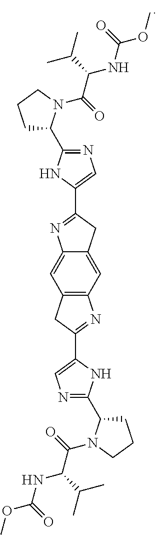

### Section VI

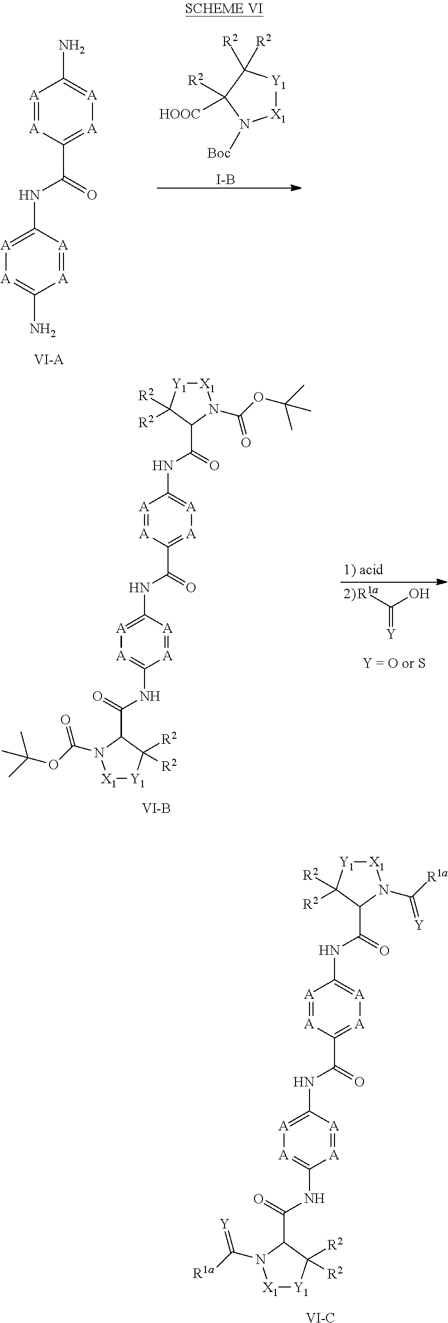

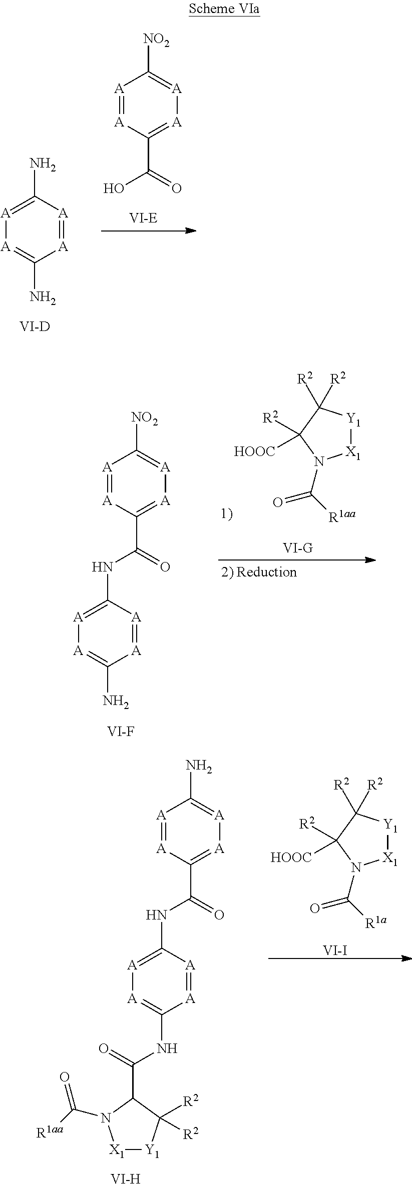

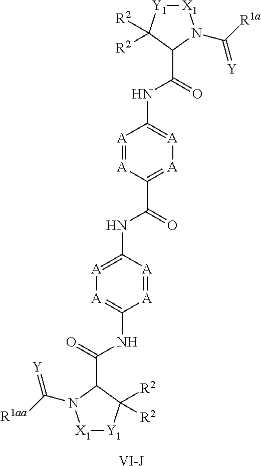

The compounds shown below in Table VI can be prepared by the methods disclosed in Scheme VI and VIa, modified as appropriate. It will be readily apparent to one of ordinary skill in the art that the compounds shown below in Table VI can be synthesized by use of the appropriate reactants, reagents and reaction conditions.

TABLE VI

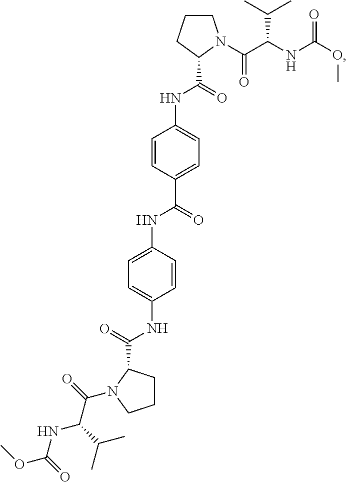

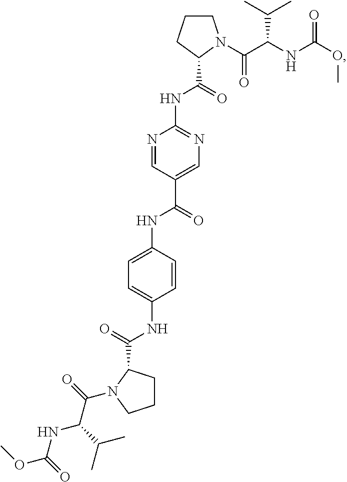

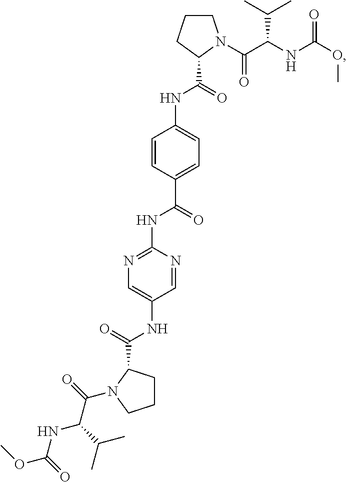

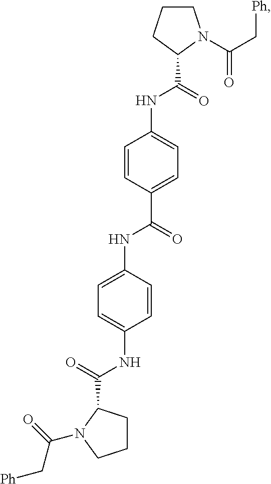

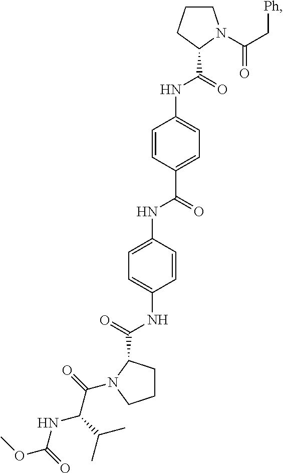

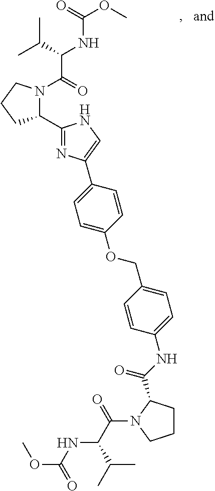

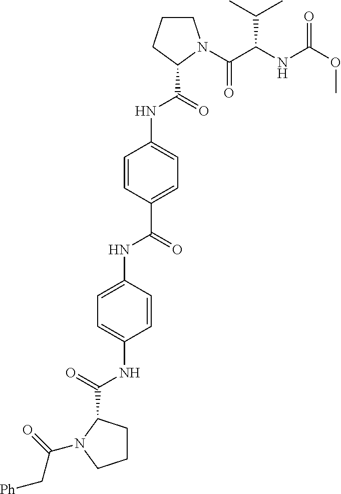

### Preparation of Compounds

### Section VI

### Example VI-I

### Preparation of Compound 101

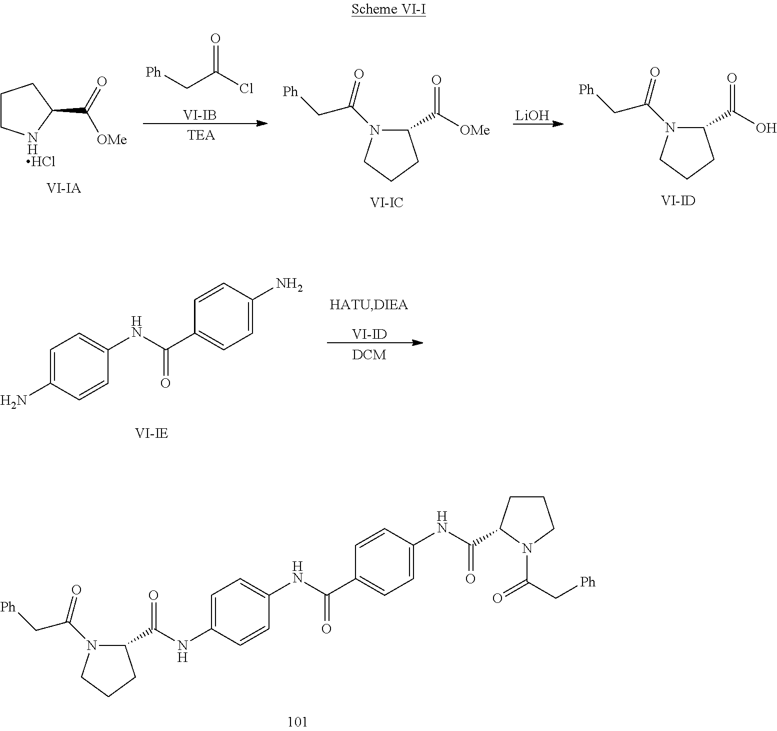

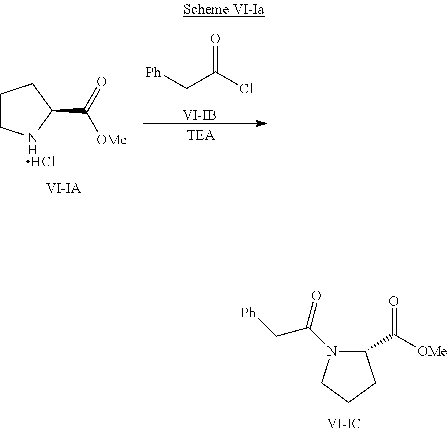

### General Procedure VI-A

To a solution of compound VI-IA (9 g, 54.5 mmol), TEA (30 mL, 218 mmol) in DCM (100 mL) was added 2-phenylacetyl chloride (VI-IB) (9.26 g, 60 mmol) in portions at 0° C. The mixture was stirred for 2 hrs at room temperature. The mixture was diluted with CH₂Cl₂ (50 mL), washed with water (50 mL×3) and brine, dried over anhydrous Na₂SO₄, and concentrated in vacuo. The resulting residue was purified by silica gel column chromatography (PE:EtOAc=4:1) to give compound VI-IC (5 g, yield 60%).

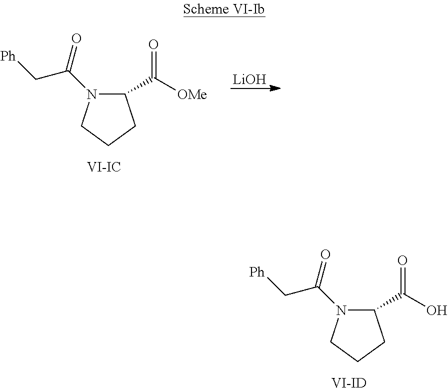

### General Procedure VI-B

To a solution of compound VI-IC (5 g, 20 mmol) in THF (40 mL) and H₂O (20 mL) was added LiOH (20 g, 80 mmol). The mixture was stirred overnight at 50° C. The mixture was concentrated and acidified with aq. HCl (1 M), and extracted with EtOAc (50 mL×3), washed with water and brine, the organic layer was dried over anhydrous Na₂SO₄, and concentrated in vacuo to give compound VI-ID (3 g, yield 64%).

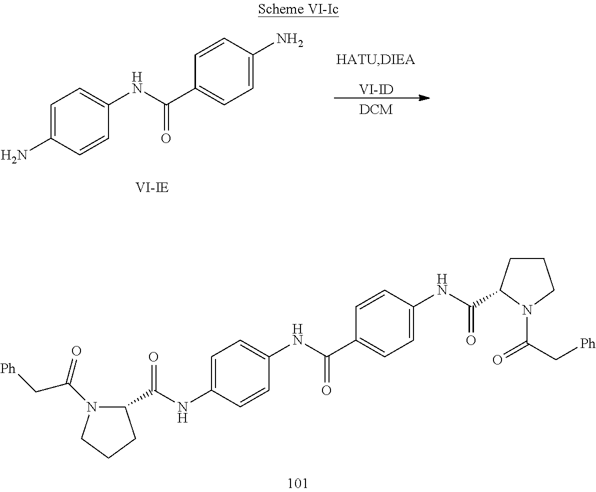

### General Procedure VI-C

To a solution of 4-amino-N-(4-aminophenyl)benzamide (VI-IE) (50 mg, 0.22 mmol) and compound VI-ID (115 mg, 0.484 mmol) in anhydrous dichloromethane (2 mL) was added HATU (251 mg, 0.66 mmol) and DIEA (171 mg, 1.32 mmol). The reaction solution was stirred at r.t for 12 hr. The mixture was washed with 5% citric acid (5 mL×2), water (5 mL×2) and brine (5 mL×2). The organic layer was dried over anhydrous Na₂SO₄, and concentrated. The residue was purified by prep-HPLC to afford compound 101 (35 mg, yield 25%) as white solid. MS (ESI) m/z (M+H)⁺ 658.1.

### Example VI-II

### Preparation of Compound 102

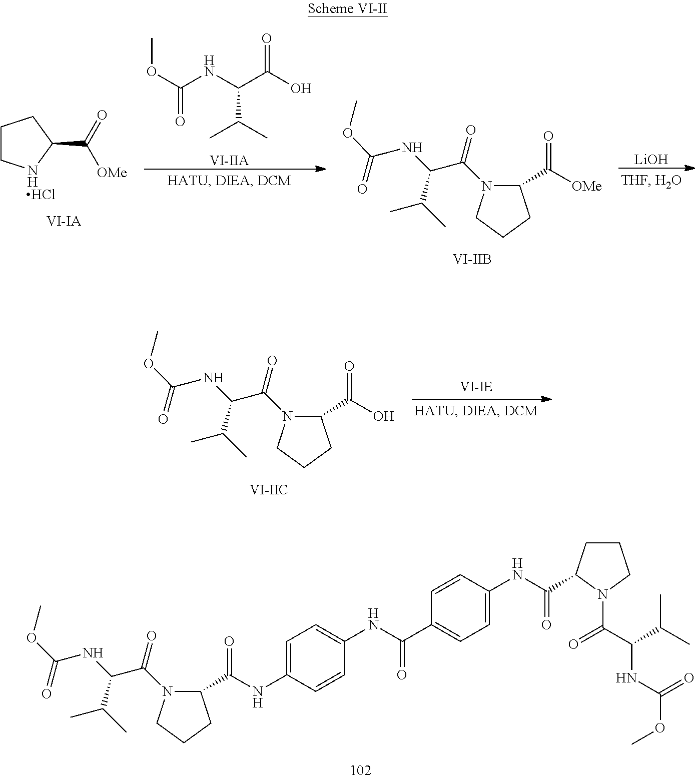

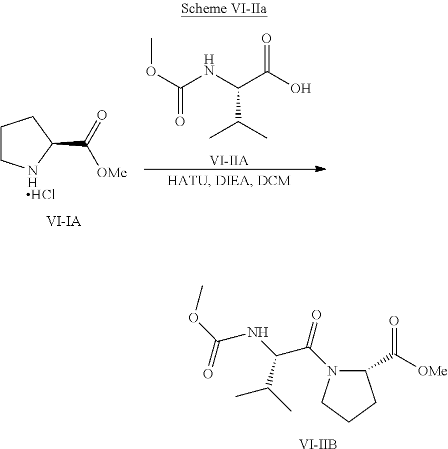

### General Procedure VI-D

Compound VI-IA (1.03 g, 6.23 mmol), compound VI-IIA (1.09 g, 6.23 mmol) and HATU (3.55 g, 9.34 mmol) was dissolved in CH₂Cl₂ (20 mL). DIEA (2.42 g, 18.69 mmol) was added and the reaction solution was stirred at r.t. for 18 hrs. The mixture was diluted with CH₂Cl₂ (50 mL), washed with water (50 mL×3) and brine, dried over anhydrous Na₂SO₄, and concentrated in vacuo. The residue was purified by silica gel column chromatography (PE:EtOAc=2:1) to give compound VI-IIB (1.63 g, yield 91%).

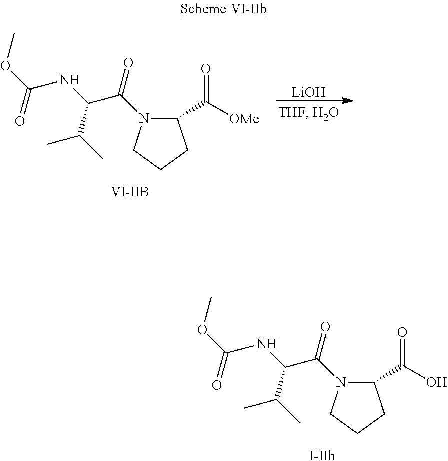

### General Procedure VI-E

To a solution of compound VI-IIB (1.63 g, 5.7 mmol) in THF (20 mL) and water (2 mL) was added LiOH (246 mg, 10.26 mmol). The mixture was stirred at r.t. for 18 hrs. The reaction mixture was acidified with aq.HCl (1 M), and extracted with EtOAc (50 mL×3), washed with water (30 mL×2) and brine (30 mL×2). The organic layer was dried over anhydrous Na₂SO₄, and concentrated in vacuo to a compound I-IIh (1.42 g, yield 90%).

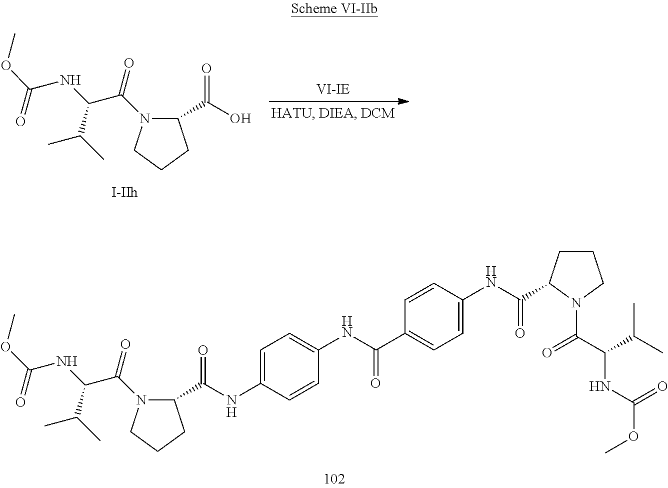

### General Procedure VI-F

To a solution of compound VI-IE (50 mg, 0.22 mmol) and compound I-IIh (132 mg, 0.484 mmol) in anhydrous dichloromethane (2 mL) was added HATU (251 mg, 0.66 mmol) and DIEA (171 mg, 1.32 mmol). The reaction solution was stirred at r.t for 12 hr. The mixture was washed with 5% citric acid (5 mL×2), water (5 mL×2) and brine (5 mL×2). The organic layer was dried over anhydrous Na₂SO₄, and concentrated. The residue was purified by prep-HPLC to afford compound 102 (80 mg, yield 49%) as white solid. MS (ESI) m/z (M+H)⁺ 736.3.

### Example VI-III

### Preparation of Compound 103

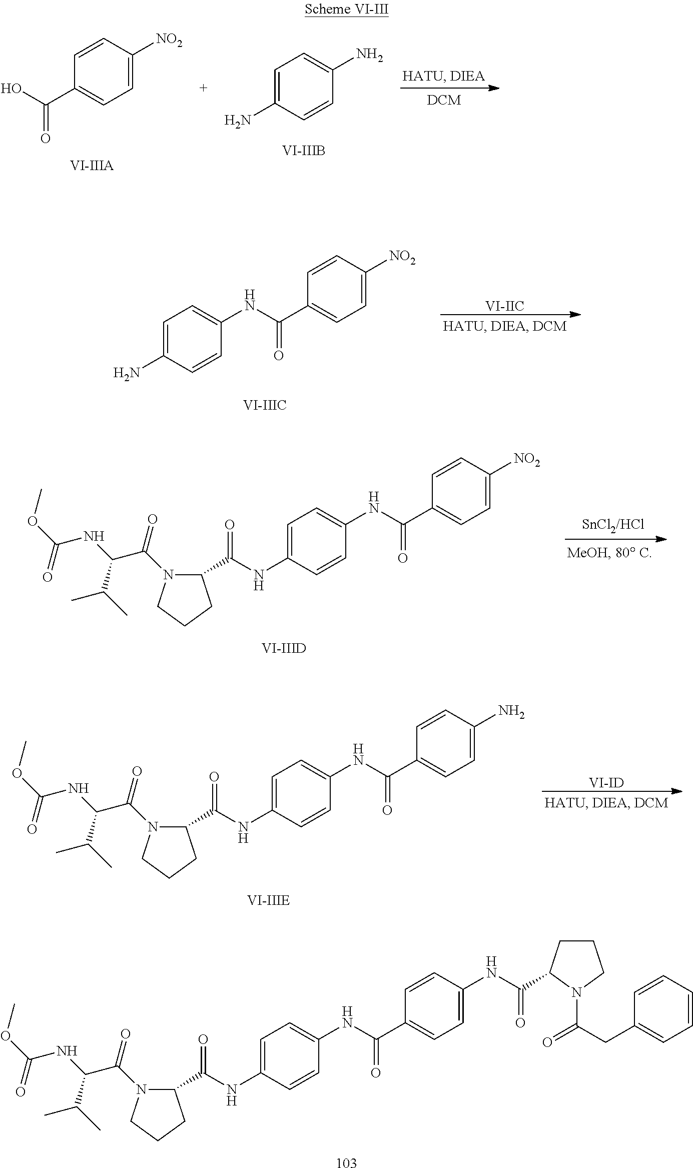

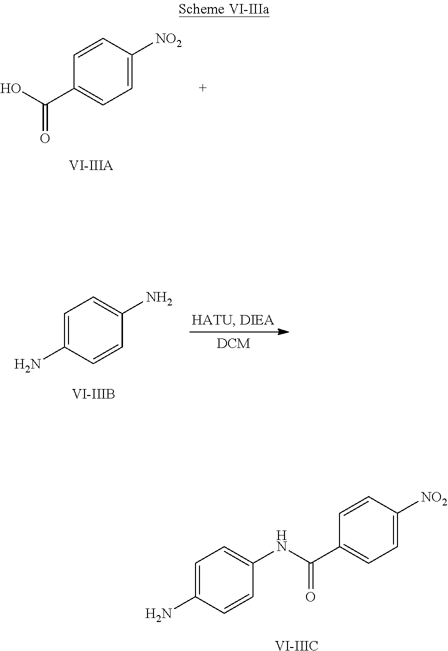

### General Procedure VI-G

To a solution of 4-nitrobenzoic acid (VI-IIIA) (1 g; 6 mmol) in anhydrous dichloromethane (100 mL) was added benzene-1,4-diamine (VI-IIIB) (640 mg, 6 mmol), HATU (2.73 g, 7.2 mmol) and DIEA (1.55 g, 12 mmol). The mixture was stirred at r.t. for 18 hrs. The mixture was diluted with CH₂Cl₂ (50 mL), washed with water (50 mL×3) and brine, dried over anhydrous Na₂SO₄, and concentrated. The residue was purified by silica gel column chromatography (PE:EtOAc=2:1) to give N-(4-aminophenyl)-4-nitrobenzamide (VI-IIIC) (1.0 g, yield 65%).

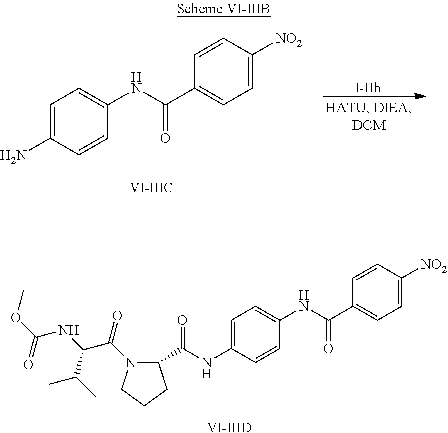

### General Procedure VI-H

To a solution of N-(4-aminophenyl)-4-nitrobenzamide (VI-IIIC) (257 mg, 1 mmol) and compound I-IIh (272 mg, 1 mmol) in anhydrous dichloromethane (4 mL) was added HATU (570 mg, 1.5 mmol) and DMA (387 mg, 3 mmol). The reaction mixture was stirred at r.t for 12 hrs. The mixture was washed with 5% citric acid (5 mL×2), water (5 mL×2) and brine (5 mL×2). The organic layer was dried over anhydrous Na₂SO₄, and concentrated. The residue was washed with Petroleum Ether (PE) to afford VI-IIID as a crude product (450 mg, yield 85%), which was used directly for the next step without further purification.

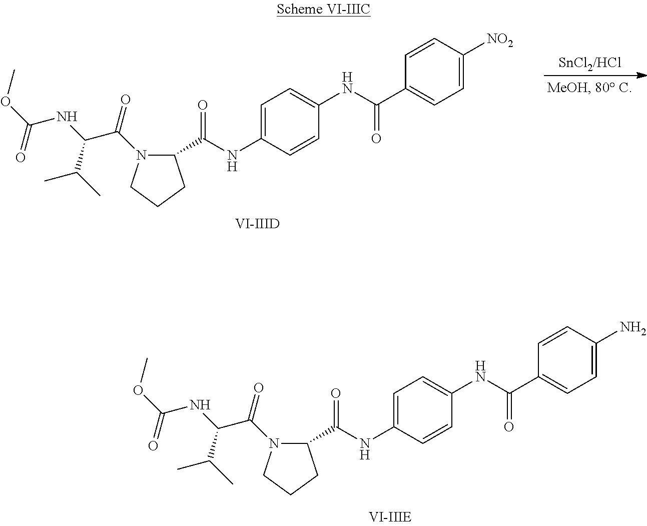

### General Procedure VI-H

To a solution of compound VI-IIID (350 mg, 0.68 mmol) in MeOH (6 mL) was added SnCl₂.H₂O (793 mg, 3.52 mmol) and conc. HCl (0.8 mL). The mixture was stirred at 80° C. for 1 h. After removal of the solvent, the reaction mixture was diluted with EtOAc (20 mL) and water (20 mL), filtered and the filtrate was extracted with EtOAc (20 mL×3). The combined organic layers were dried over anhydrous MgSO₄ and concentrated to afford VI-IIIE as a crude product (210 mg, yield 64%). MS (ESI) m/z (M+H)⁺ 482.1.

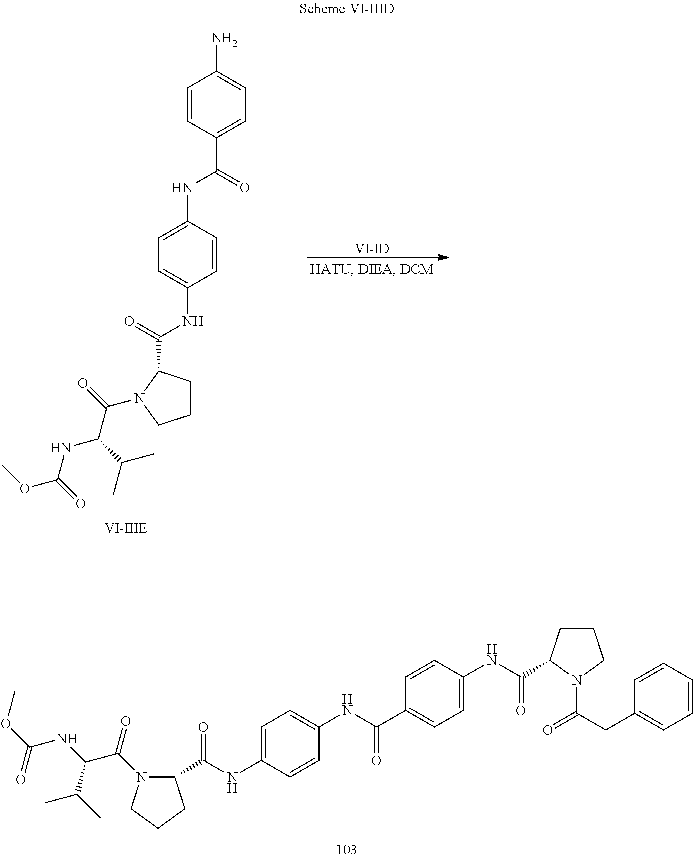

### General Procedure VI-I

The procedure for the preparation of compound 103 is similar to that of preparation of compound 102 as described in General Procedure VI-F. 120 mg, yield 40%, white solid. MS (ESI) m/z (M+H)⁺ 697.5.

### Example VI-IV

### Preparation of Compound 104

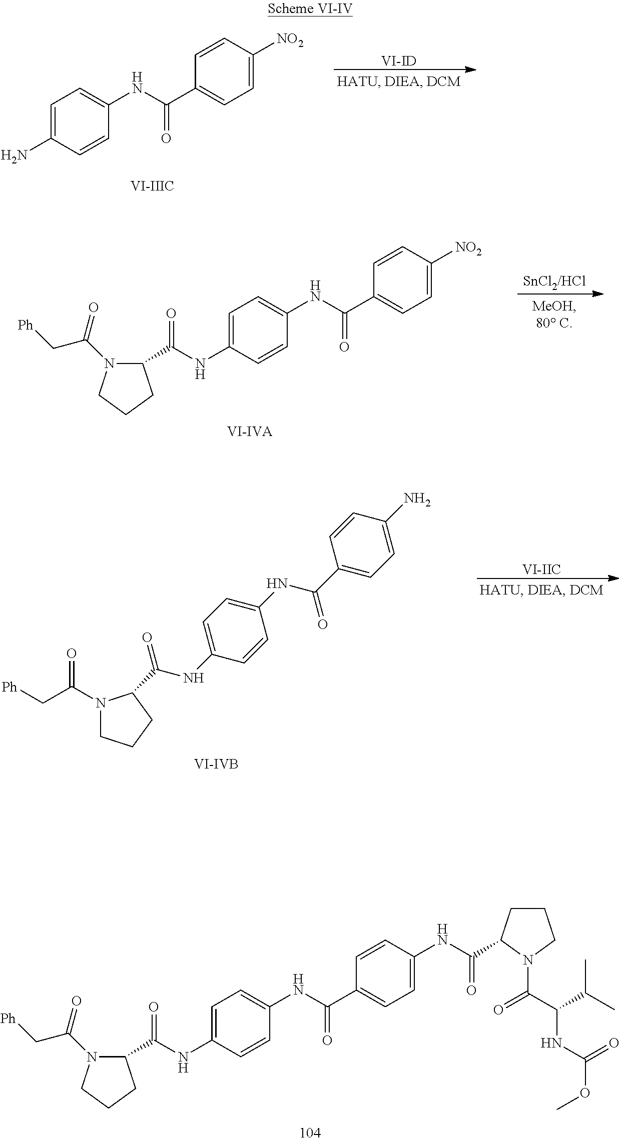

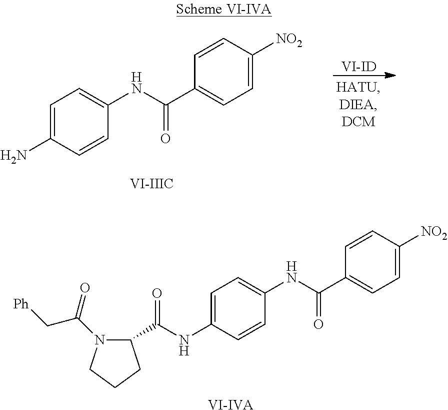

### General Procedure VI-I

To a solution of N-(4-aminophenyl)-4-nitrobenzamide (VI-IIIC) (200 mg, 0.86 mmol) in anhydrous dichloromethane (20 mL) was added compound VI-ID (222 mg, 0.86 mmol), HATU (655 mg, 1.72 mmol) and DIEA (556 mg, 4.3 mmol). The mixture was stirred at 0° C. for 30 min and then was allowed to warm to r.t and stirred for 11 hrs. The mixture was extracted with EtOAc (100 mL×3) and saturated aq. NaHCO₃ (20 mL×3). The organic layer was dried over anhydrous Na₂SO₄, and concentrated. The residue was purified by prep-TLC (EtOAc as eluent) to give compound VI-IVA (200 mg, yield 49%). MS (ESI) m/z (M+H)⁺ 473.

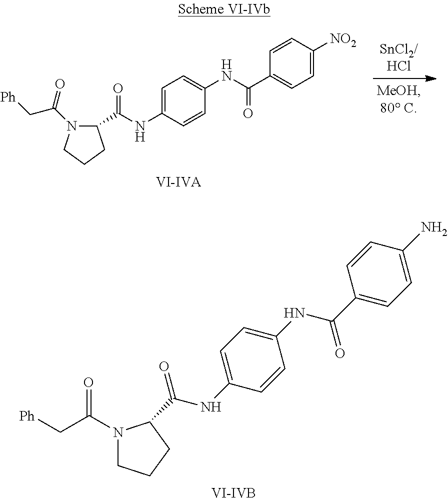

### General Procedure VI-J

To a solution of compound VI-IVA (200 mg, 0.41 mmol) in MeOH (20 mL) was added SnCl₂.H₂O (366 mg, 1.6 mmol) and conc. HCl (0.4 mL). The mixture was stirred at 0° C. for 30 min and then warmed to 85° C. for 1 hour. The mixture was cooled to r.t., extracted with EtOAc (100 mL×3) and saturated aq.NaHCO₃ (20 mL×3), washed with water (50 mL×2), the organic layer was dried over anhydrous Na₂SO₄, and concentrated to give VI-IVB (100 mg, yield 55%). MS (ESI) m/z (M+H)⁺ 443.

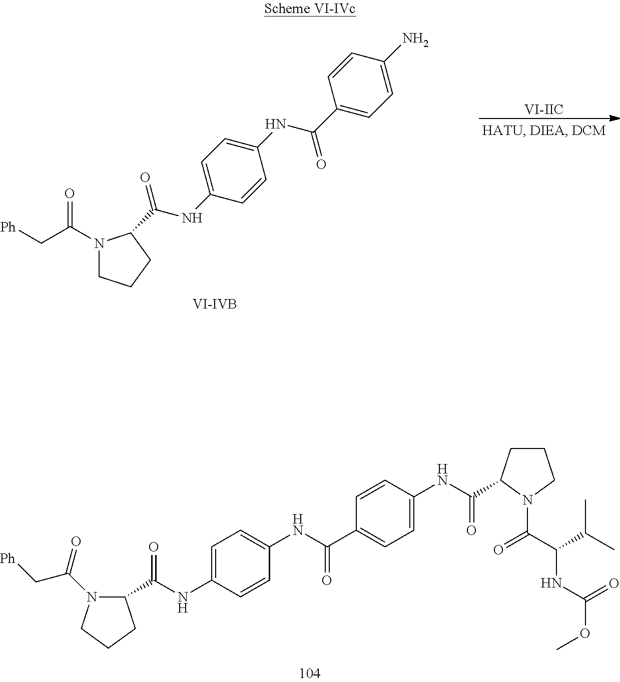

### General Procedure VI-K

The procedure for the preparation of compound 104 is similar to that of preparation of compound 102 as described in General Procedure VI-F. 26 mg, yield 46%, white solid. MS (ESI) m/z (M+H)⁺ 697.3.

### Example VI-V

### Preparation of Compound 105

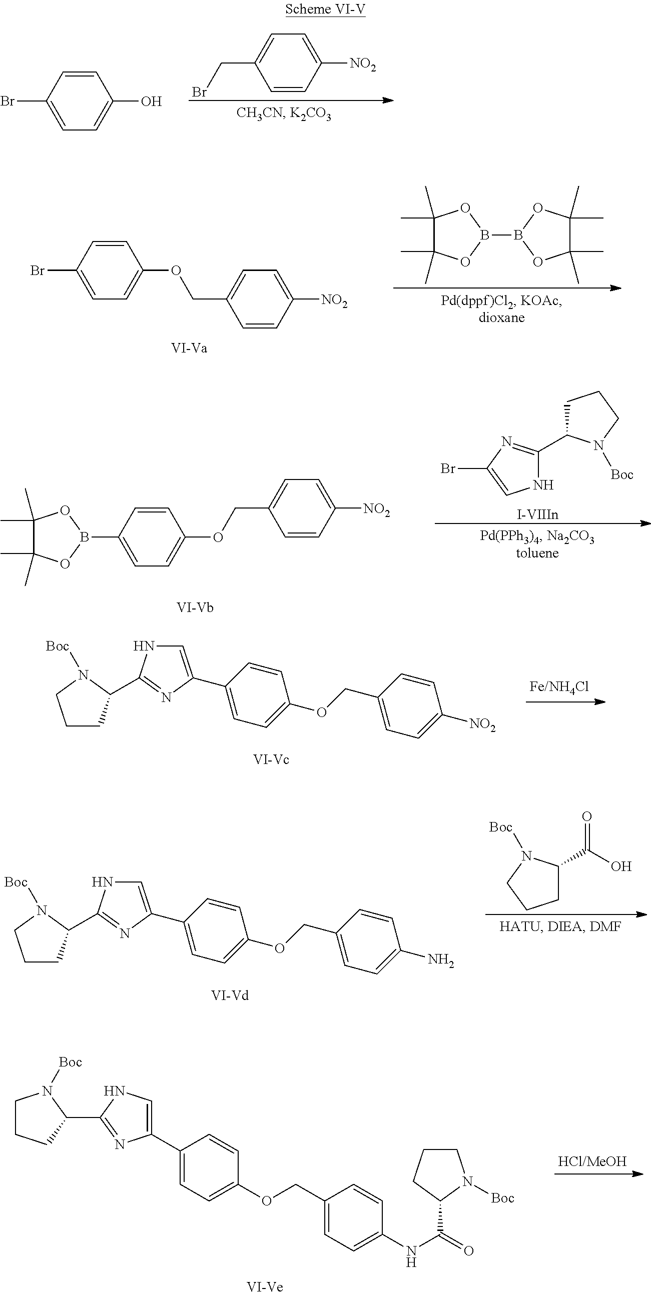

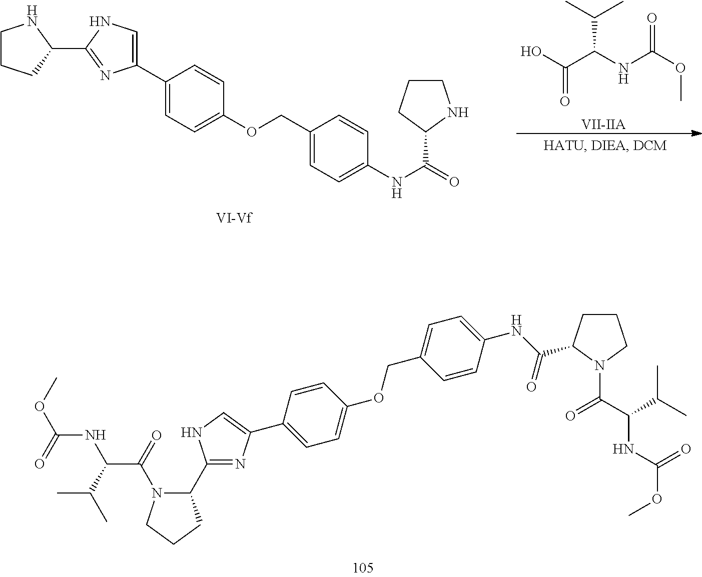

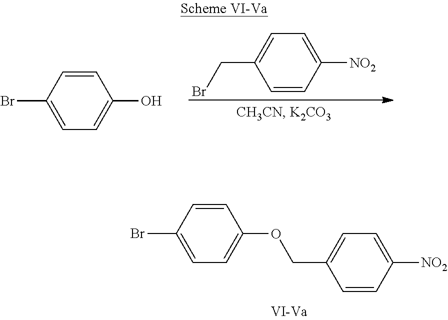

### General Procedure VI-L

To a mixture of 4-bromophenol (10.8 g, 0.05 mol) and K₂CO₃ (20.73 g, 0.15 mol) in CH₃CN (200 mL) was added 4-nitrobenzyl bromide (10.8 g, 0.05 mol) with stirring at room temperature. The reaction mixture was heated to reflux for 7 hours. TLC (petroleum ether/EtOAc=10:1) showed the reaction was complete. After being cooled to r.t. the mixture was filtered. The filtrated was concentrated to afford compound VI-Va (11 g, yield 71.4%), which was used in the next step without further purification.

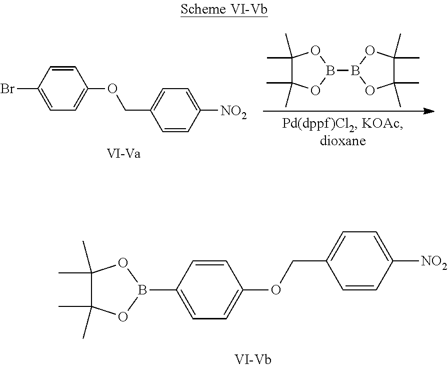

### General Procedure VI-M

To a mixture of compound VI-Va (3.08 g, 0.01 mol), bis(pinacolato)diboron (2.54 g, 0.01 mol) and KOAc (2.94 g, 0.03 mol) in dioxane (30 mL) was added Pd(dppf)Cl₂ (0.73 g, 0.001 mol) under N₂ atmosphere protection. The resulted mixture was stirred at 100-110° C. overnight. TLC (petroleum ether/EtOAc=5:1) indicated disappearance of starting material. The solvent was distilled under reduced pressure. Water (20 mL) was added into the residue and extracted with EtOAc (50 mL×3). The combined organic layers were concentrated and the crude product was purified by column chromatography on silica gel to give compound VI-Vb (2.1 g, yield 59%) as a yellow solid. ¹H NMR (400 MHz, CDCl₃) δ 8.24 (d, J=8.8 Hz, 2H), 7.77 (d, J=8.8 Hz, 2H), 7.60 (d, J=8.8 Hz, 2H), 6.96 (d, J=8.8 Hz, 2H), 5.2 (s, 2H), 1.34 (s, 12H).

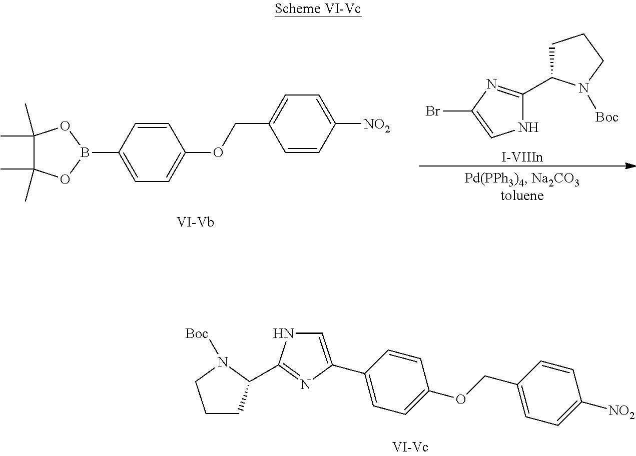

### General Procedure VI-N

To a solution of compound VI-Vb (1.0 g, 2.82 mmol), compound I-VIIIn (0.89 g, 2.82 mmol) and Na₂CO₃ (0.9 g, 8.46 mmol) in toluene/H₂O (20 mL/2 mL) was added Pd(PPh₃)₄ (0.35 g, 0.3 mmol) under nitrogen in one portion. The mixture was heated to reflux and stirred overnight. TLC (petroleum ether/EtOAc=2:1) showed that the reaction was complete. The mixture was concentrated in vacuo. The residue was portioned between EtOAc and water. The combined organic layers were washed with brine, dried over anhydrous Na₂SO₄, filtered and concentrated. The crude product was purified by column chromatography on silica gel to afford compound VI-Vc (0.3 g, yield 23%) as a white solid.

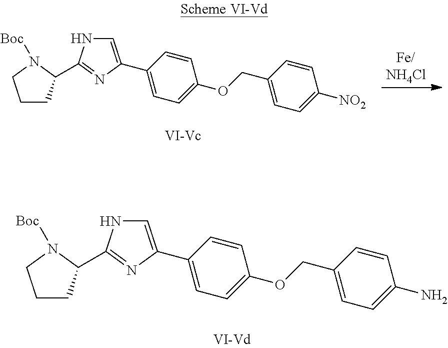

### General Procedure VI-O

To a mixture of compound VI-Vc (0.15 g, 0.33 mmol), NH₄Cl (0.14 g, 2.64 mmol) in dioxane/CH₃OH/H₂O (18 mL/12 mL/6 mL) was added iron powder (0.09 g, 1.65 mmol) with stirring at room temperature. Then the reaction mixture was warmed to reflux. After 1.5 hours, the mixture was cooled to room temperature and adjusted pH>7 by sat. aq. NaHCO₃, extracted with EtOAc. The organic phase was washed with brine, dried over Na₂SO₄, filtered and concentrated under reduced pressure. The crude product VI-Vd was used in the next step without further purification.

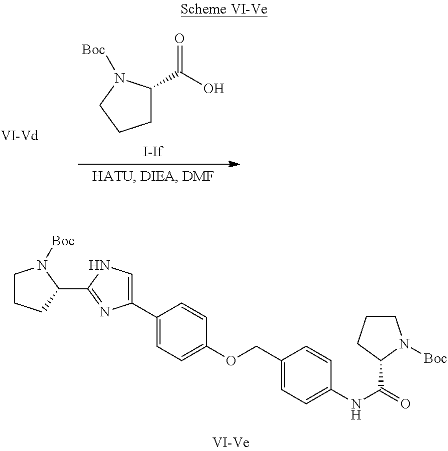

### General Procedure VI-P

A mixture of compound VI-Vd (0.23 g, 0.53 mmol), N-Boc-L-proline (I-If, 0.11 g, 0.53 mmol), HATU (0.4 g, 1.06 mmol), DIEA (0.14 g, 1.06 mmol) in DMF (20 mL) was stirred at room temperature overnight. The mixture was diluted with EtOAc (20 mL) and washed with brine. The organic layer was separated, dried and concentrated under reduced pressure. The residue was purified by column chromatography on silica gel to give compound VI-Ve (0.13 g, yield 39%).

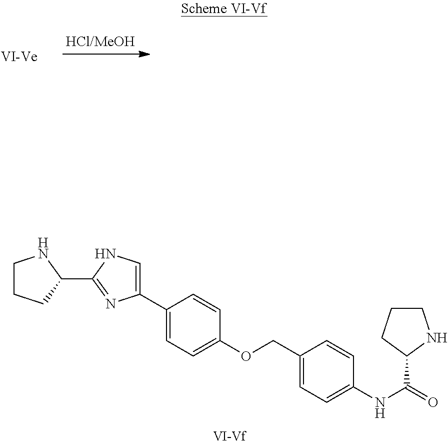

### General Procedure VI-Q

A mixture of compound VI-Ve (0.13 g, 0.2 mmol) in MeOH/HCl (5 mL) was stirred at room temperature for 30 minutes, then concentrated under reduced pressure. The crude product VI-Vf was used in the next step without further purification.

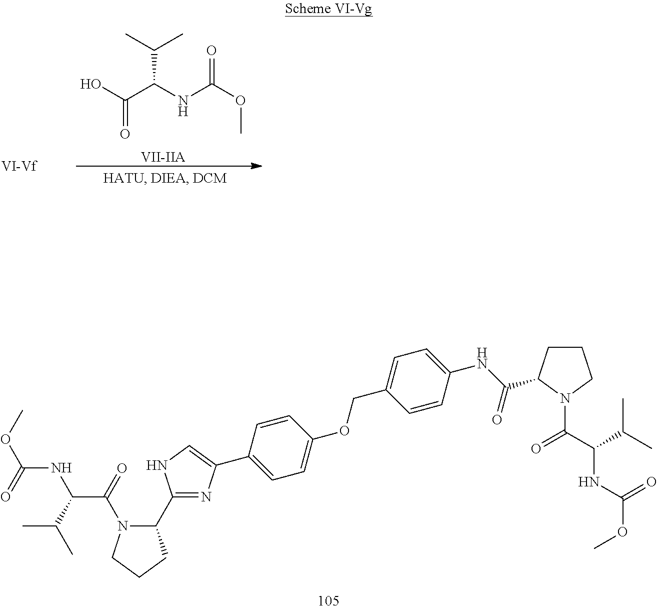

### General Procedure VI-R

To a solution of compound VI-Vf (0.1 g, 0.23 mmol) in CH₃CN (2 mL) was added compound VII-IIA (0.08 g, 0.46 mmol), EDC.HCl (0.107 g, 0.55 mmol), DIPEA (0.072 g, 0.0.55 mmol) and HOBt (0.075 g, 0.55 mmol). The reaction mixture was stirred at room temperature overnight. Then the mixture was diluted with DCM, washed with water and brine, dried over anhydrous Na₂SO₄, filtered and concentrated under reduced pressure. The crude product was purified by Prep-TLC (eluted by petroleum ether/EtOAc=1:2) to afford compound 105 (0.005 g, yield: 3%) as a yellow solid. ¹H NMR (400 MHz, CD₃OD) δ 7.49-7.43 (m, 4H), 7.29 (d, J=8.4 Hz, 2H), 7.05 (s, 1H), 6.88 (d, J=8.4 Hz, 2H), 4.95 (s, 2 H), 4.72 (s, 2H), 4.53-4.42 (m, 2H), 3.92-4.12 (m, 2H), 3.91-3.71 (m, 2H), 3.19-3.11 (m, 6 H), 2.23-1.89 (m, 10H), 1.24-0.89 (m, 12H). MS (ESI) m/z (M+H)⁺ 746.4.

### Section VII

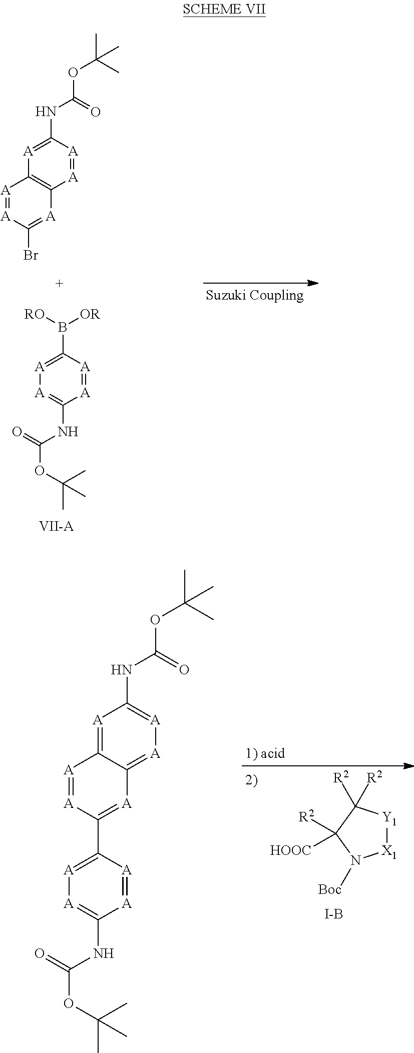

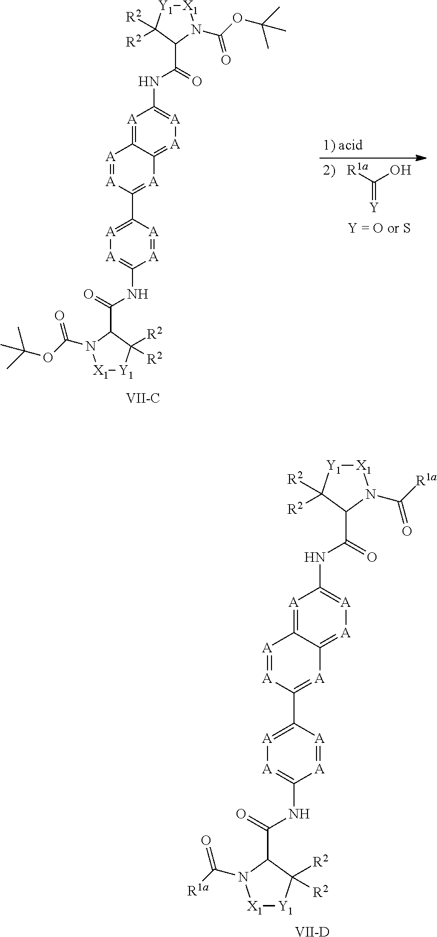

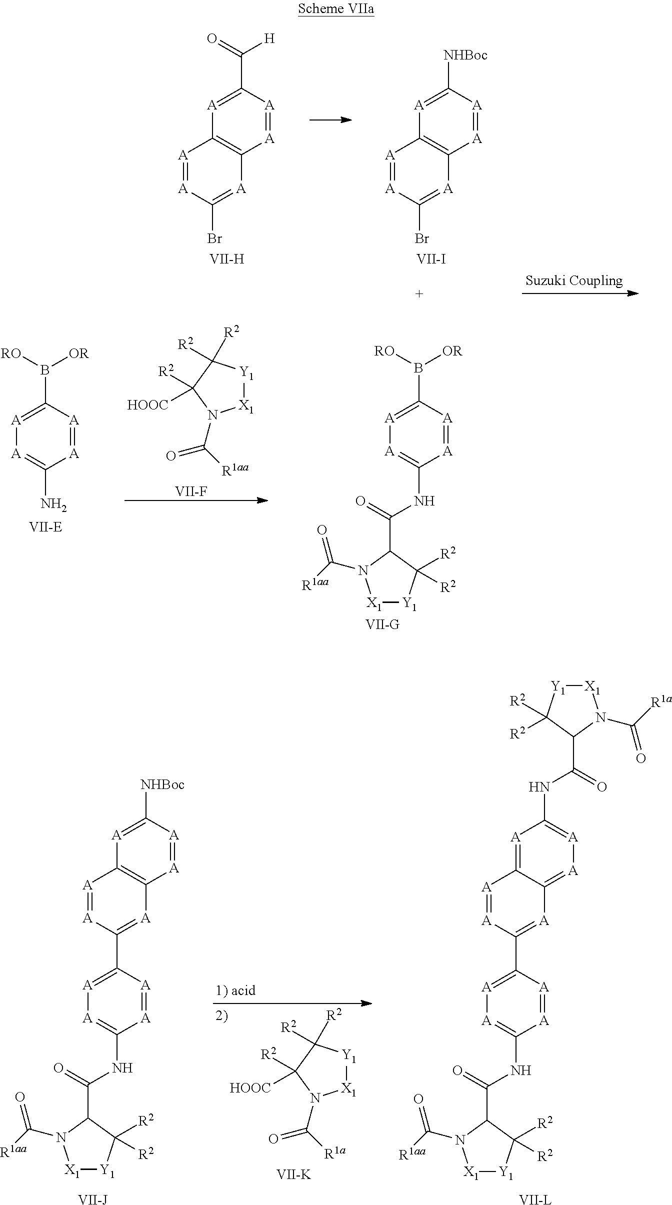

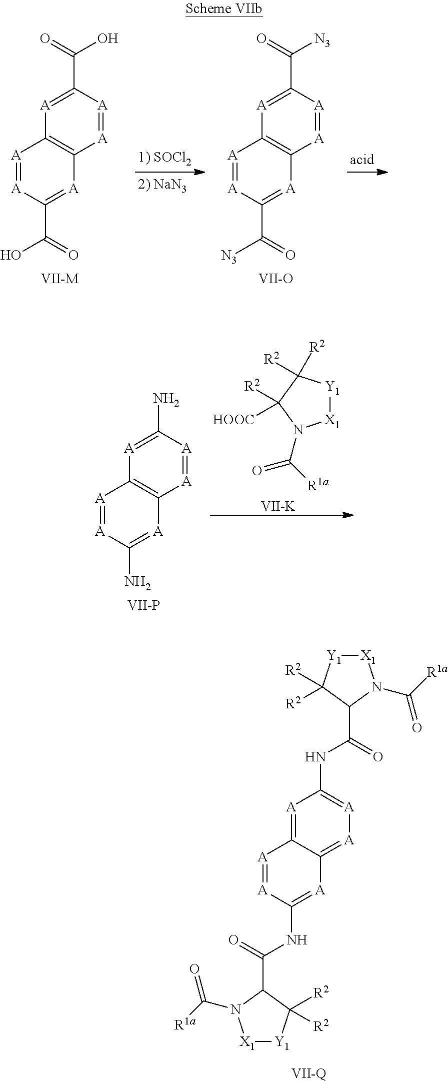

The compounds shown below in Table VII can be prepared by the methods disclosed in Scheme VII, VIIa and VIIb, modified as appropriate. It will be readily apparent to one of ordinary skill in the art that the compounds shown below in Table VII can be synthesized by use of the appropriate reactants, reagents and reaction conditions.

TABLE VII

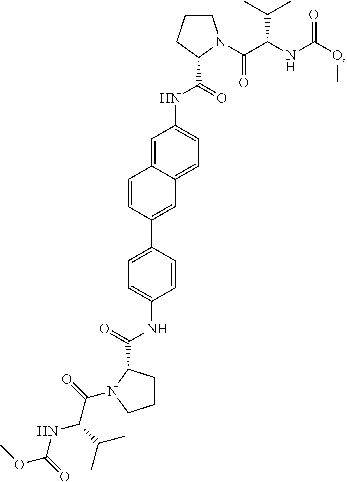

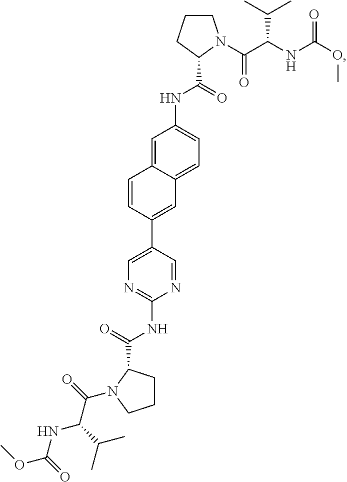

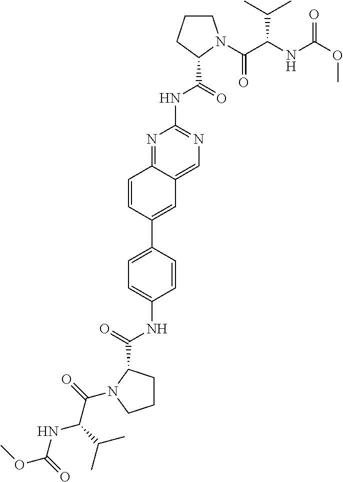

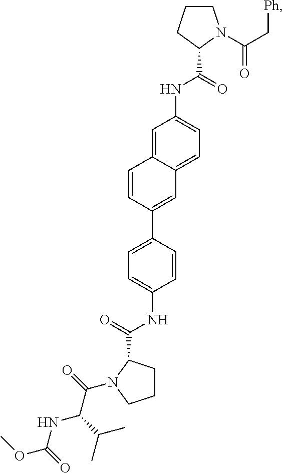

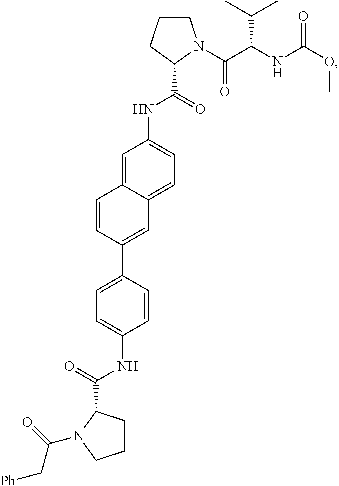

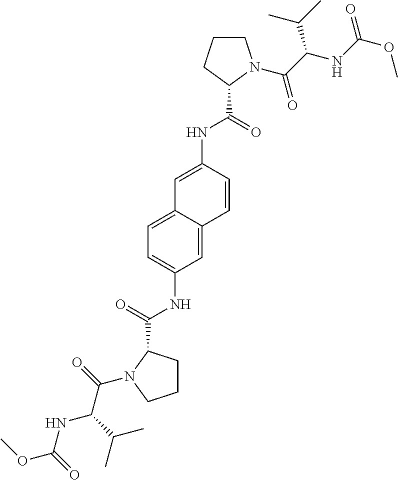

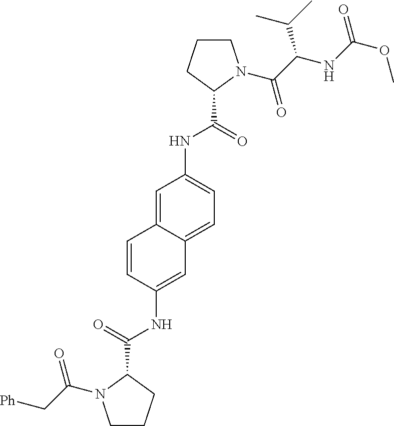

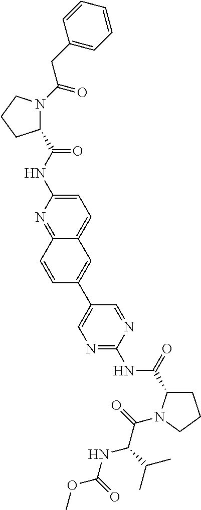

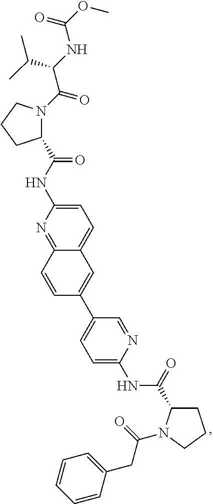

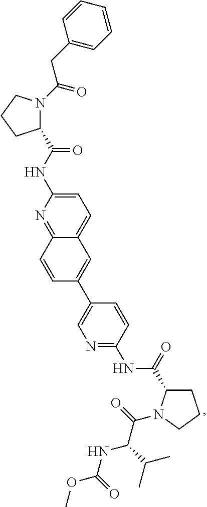

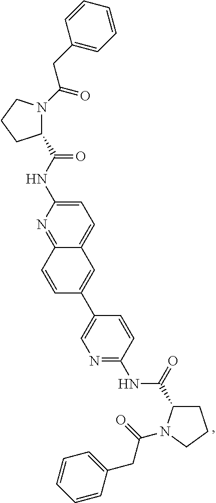

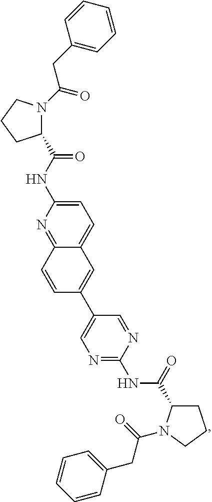

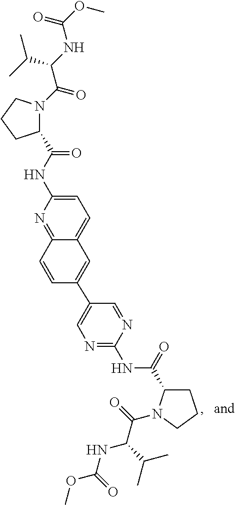

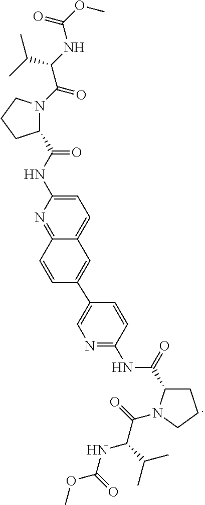

### Preparation of Compounds

### Section VII

### Example VII-I

### Preparation of Compound 201 and 202

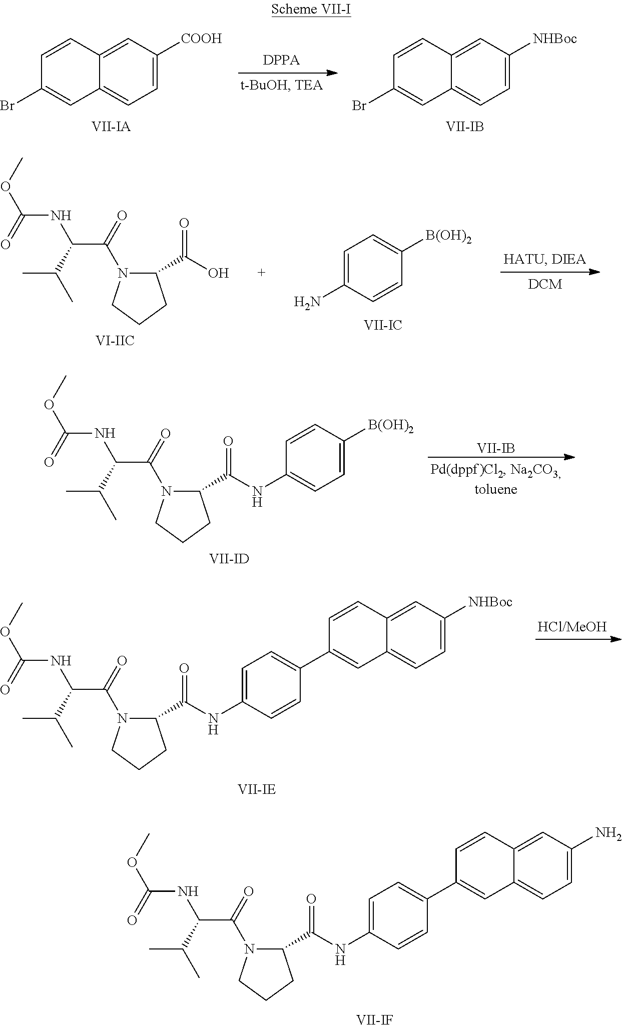

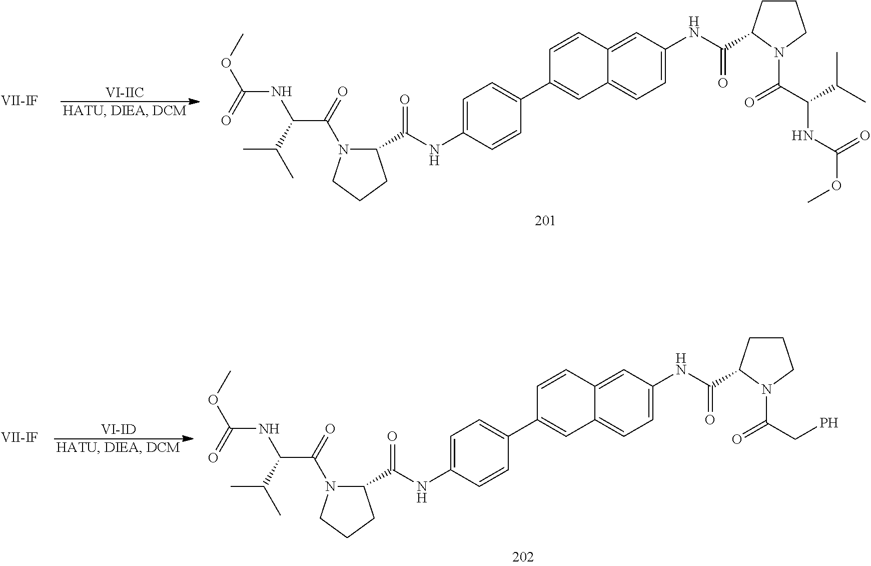

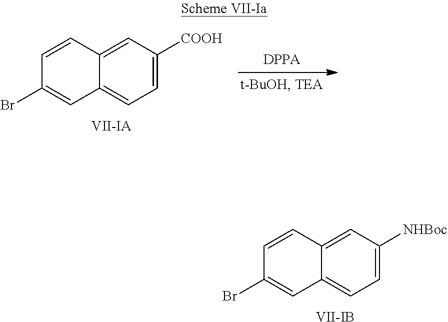

### General Procedure VII-A

6-Bromonaphthalene-2-carboxylic acid (VII-IA) (11 g, 44 mmol) in t-BuOH (50 mL) containing Et₃N (4.86 g, 48 mmol) was treated with DPPA (13.2 g, 48 mmol) and stirred at 100° C. overnight. After cooling to r.t., the mixture was poured into water and extracted with EtOAc (100 mL×3), the organic layer was combined and washed with brine, dried over anhydrous sodium sulfate, concentrated in vacuo. The residue was purified on silica gel column chromatography, eluting by petroleum ether and ethyl acetate (7:1), to afford compound VII-IB (12 g, yield 85%).

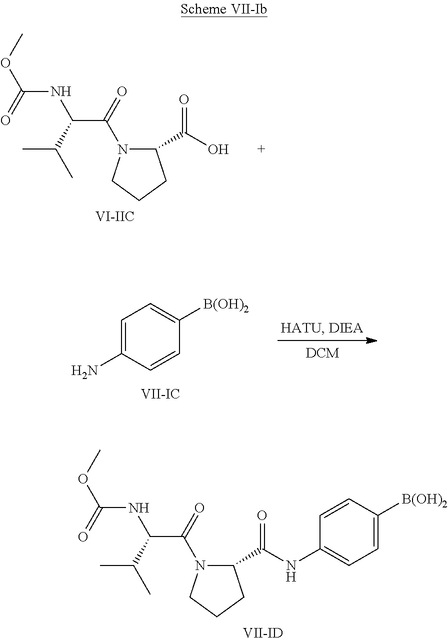

### General Procedure VII-B

To a solution of compound 4-aminophenylboronic acid (VII-IC) (101 mg, 0.74 mmol) in anhydrous dichloromethane (5 mL) was added compound VI-IC (200 mg, 0.74 mmol), HATU (421 mg, 1.11 mmol) and DIEA (320 mg, 2.5 mmol). The mixture was stirred at r.t. for 4 hrs. After completion of the reaction, the mixture was extracted with EtOAc (100 mL×2), and water (20 mL×2). The organic layer was dried over anhydrous Na₂SO₄, and concentrated. The residue was purified by prep-TLC (MeOH/EA=10:1) to give compound VII-ID (240 mg, yield 83%). MS (ESI) m/z (M+H)⁺ 392.

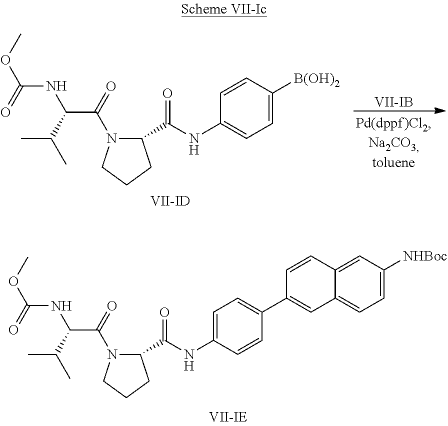

### General Procedure VII-C

To a solution of compound VII-ID (240 mg, 0.61 mmol) in toluene (8 mL) was added aq. Na₂CO₃ (2 M, 1.53 mL), compound VII-IB (195 mg, 0.61 mmol), and Pd(dppf)Cl₂ (27 mg, 0.03 mmol). The flask was purged with nitrogen and the mixture was heated at reflux for 4 hrs. The reaction was monitored by LCMS. Then the mixture was cooled to r.t. and extracted with EtOAc (100 mL×2), and washed with brine, the organic layer was dried over anhydrous Na₂SO₄, and concentrated in vacuo. The residue was purified by prep-TLC (EtOAc as eluent) to give compound VII-IE (200 mg, yield 56%). MS (ESI) m/z (M+H)⁺ 589.

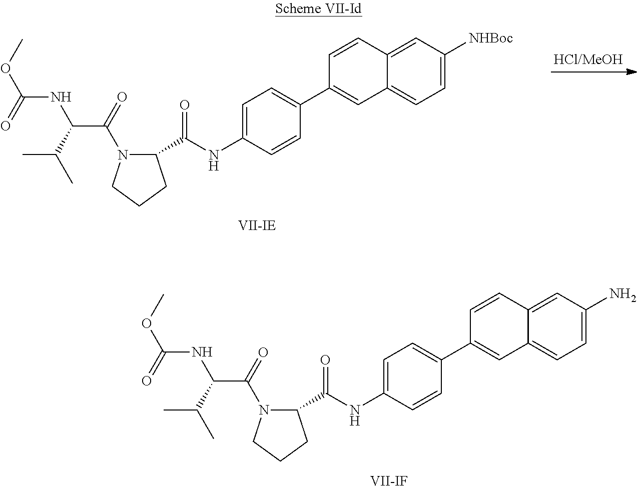

### General Procedure VII-D

Compound VII-IE (200 mg, 0.34 mmol) was dissolved in a solution of HCl (gas) in MeOH (4 M, 5 mL) and the mixture was heated at 50° C. for 2 hrs. After completion of the reaction, the mixture was concentrated under reduced pressure and then neutralized with saturated aq. NaHCO₃. The mixture was extracted three times with EtOAc. The organic layer was dried over anhydrous Na₂SO₄ and concentrated in vacuo to give a crude product VII-IF, which was used directly in the next step (138 mg, yield 83%). MS (ESI) m/z (M+H)⁺ 489.

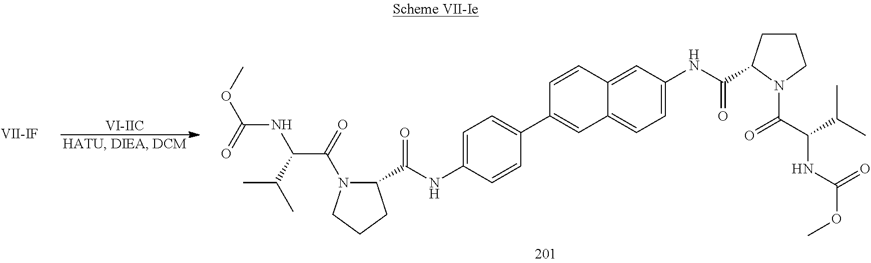

### General Procedure VII-E

The procedure for the preparation of compound 201 is similar to that of preparation of compound 102 as described in General Procedure VI-F. 58 mg, yield 56%. Yellow Solid. MS (ESI) m/z (M+H)⁺ 743.4.

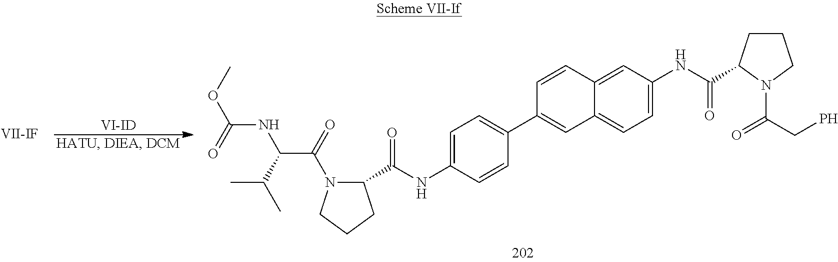

### General Procedure VII-F

The procedure for the preparation of compound 202 is similar to that of preparation of compound 102 as described in General Procedure VI-F. 46 mg, yield 47%. Yellow Solid. MS (ESI) m/z (M+H)⁺ 704.4.

### Example VII-II

### Preparation of Compound 203

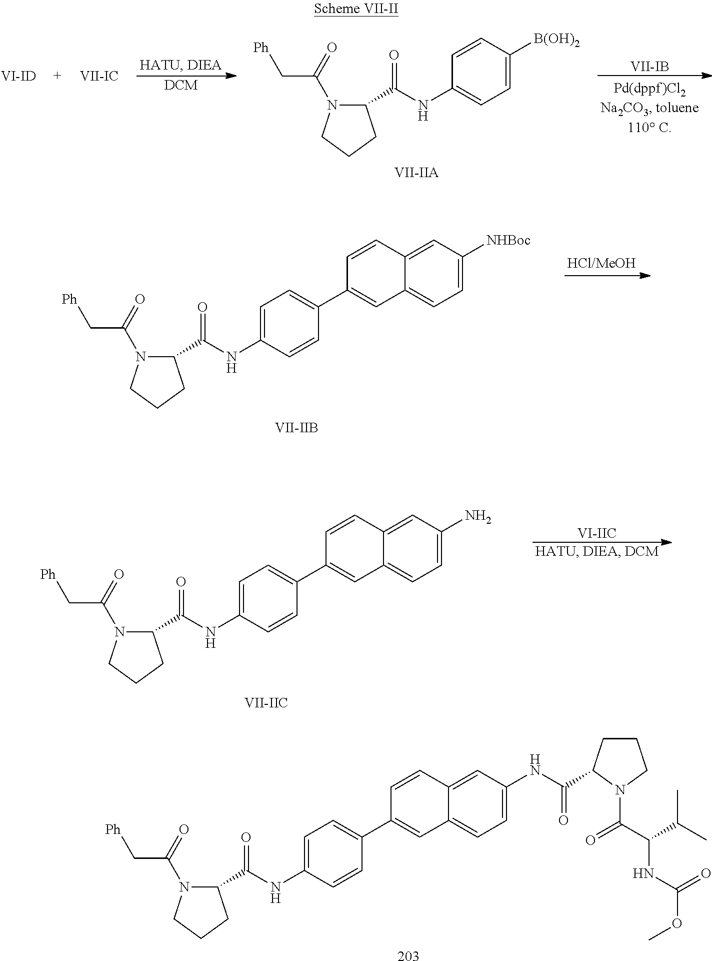

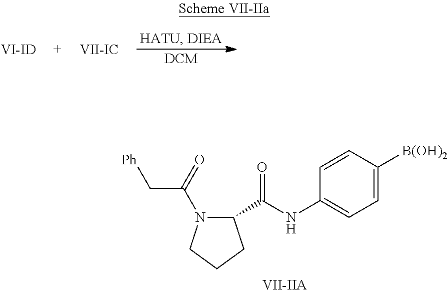

### General Procedure VII-G

A flask (100 mL) was charged with compound VI-ID (500 mg, 2.15 mmol) and anhydrous CH₂Cl₂ (30 mL). To the solution was added HATU (1.22 g, 3.2 mmol) DIEA (1.11 g, 8.6 mmol), and 4-aminophenylboronic acid (VII-IC) (440 mg, 3.2 mmol). The resulting mixture was stirred at room temperature for 17 hrs. After the material was consumed, the mixture was concentrated, diluted with EtOAc (150 mL), washed with water and brine, dried over sodium sulfate, concentrated in vacuo to give a yellow oil. It was isolated by silica gel column chromatography (eluted with MeOH:EtOAc=1:1) to afford compound VII-IIA as a yellow solid (700 mg, yield 93%).

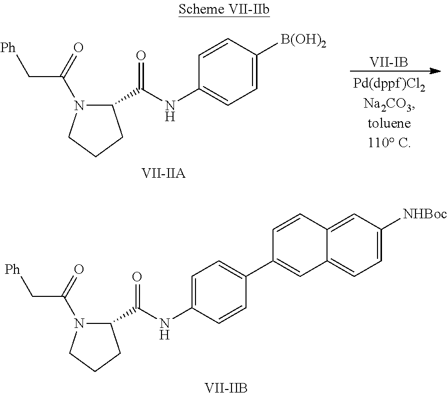

### General Procedure VII-H

A flask was charged with compound (400 mg, 1.136 mmol), compound VII-IB (366 mg 1.136 mmol), Pd(dppf)Cl₂ (50 mg, 0.068 mmol) and aq. Na₂CO₃ (2 M, 2.8 mL, 5.68 mmol), toluene (10 mL). The flask was purged with nitrogen, after that, the mixture was heated under reflux for 4 hours. LCMS showed the reaction was completed. The mixture was cooled to r.t., extracted with EtOAc (50 mL×3), the combined extracts was dried over sodium sulfate, filtered and concentrated in vacuo to give the crude product. It was purified by prep-TLC to give compound VII-IIB as white solid. (260 mg, yield 42%).

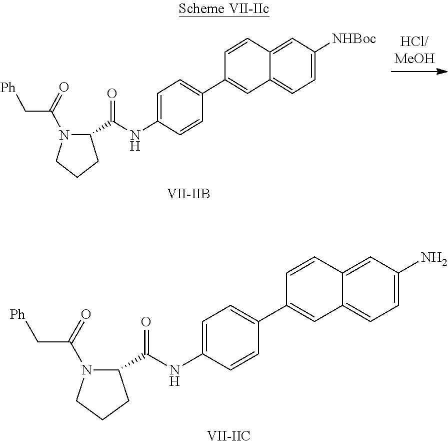

### General Procedure VII-I

Compound VII-IIB (260 mg, 0.47 mmol) was dissolved in a solution of HCl (gas) in MeOH (4 M, 5 mL) and the mixture was stirred at 40° C. for 2 hours. LCMS showed the reaction was completed. The mixture was concentrated under reduced pressure and then neutralized with saturated aq. NaHCO₃. The mixture was extracted three times with EtOAc. The organic layer was dried over anhydrous Na₂SO₄ and concentrated in vacuo to give compound VII-IIC as a crude product (200 mg, 94%), which was used directly in next step.

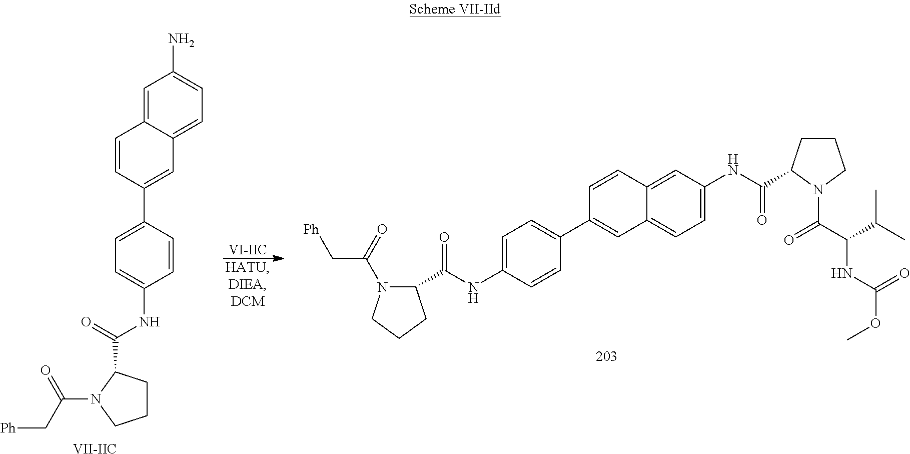

### General Procedure VII-J

The procedure for the preparation of compound 203 is similar to that of preparation of compound 102 as described in General Procedure VI-F. 25 mg, yield 18%. Light Yellow Solid. MS (ESI) m/z (M+H)⁺ 704.1.

### Example VII-III

### Preparation of Compound 204

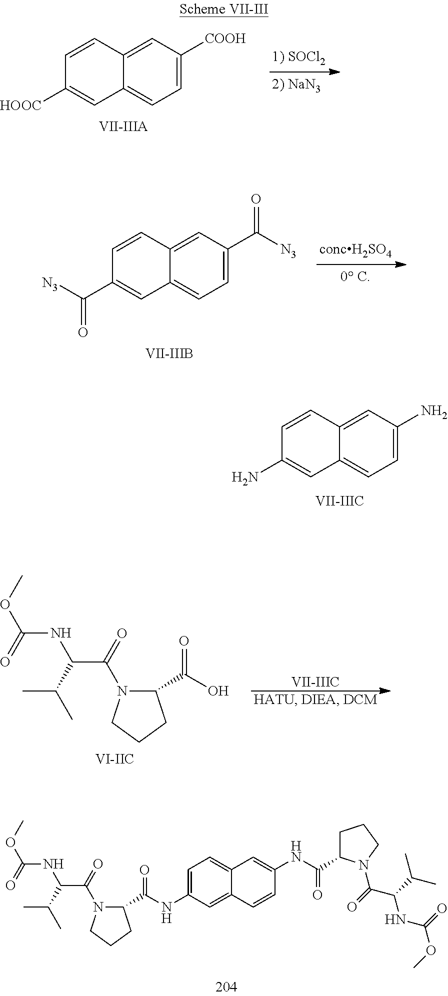

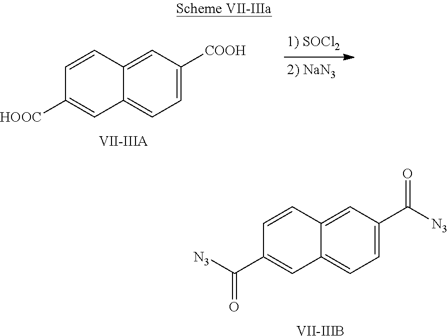

### General Procedure VII-K

Naphthalene-2,6-dicarboxylic acid (VII-IIIA) (2.2 g, 10.2 mmol) was dissolved in 20 mL of SOCl₂ and the mixture was refluxed for 4 hrs. After completion of the reaction, the mixture was concentrated under reduced pressure. The residue was dissolved in 400 mL of acetone and was added into the solution of NaN₃ (2.585 g, 39.73 mmol) in 50 mL of water at 0° C. The reaction was stirred at r.t. overnight. The precipitate formed was filtered, washed with water and dried to give compound VII-IIIB (2.48 g, yield 94%). ¹H NMR (DMSO-d₆, 400 MHz) δ 8.76 (s, 2H), 8.35 (d, J=8.8 Hz, 2H), 8.07 (d, J=8.4 Hz, 2H).

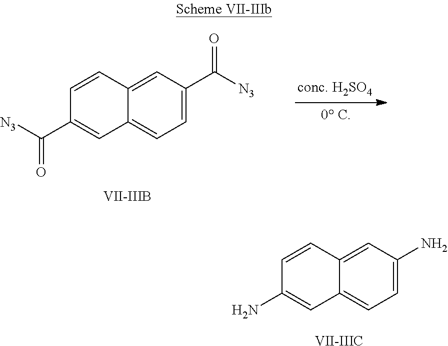

### General Procedure VII-L

Compound VII-IIIB (1.5 g, 5.64 mmol) was added into 45 mL of conc. H₂SO₄ at 0° C. portion wise. After addition, the reaction solution was stirred at r.t. for 2 hrs. The solution was poured into ice water (20 mL) slowly and made alkaline by addition of aq. NaOH (50%). The mixture was extracted with EtOAc (100 mL×3). The combined organic layers were washed with brine, dried over anhydrous Na₂SO₄, and concentrated in vacuo to give naphthalene-2,6-diamine (VII-IIIC) (411 mg, yield 46%), which was used directly in next step without further purification.

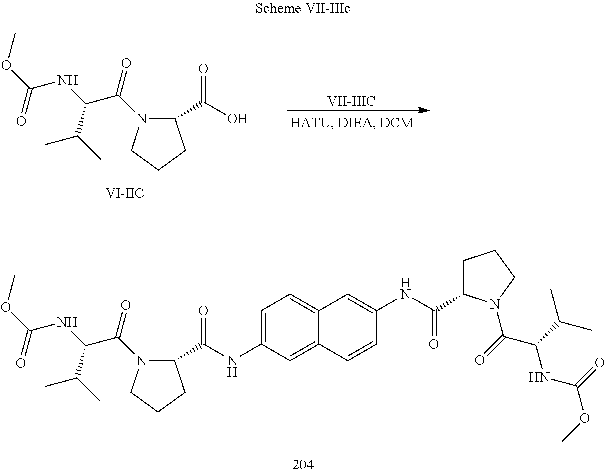

### General Procedure VII-M

The procedure for the preparation of compound 204 is similar to that of preparation of compound 102 as described in General Procedure VI-F. 62 mg, yield 16%. Pale-red solid. MS (ESI) m/z (M+H)⁺ 667.2.

### Example VII-IV

### Preparation of Compound 205

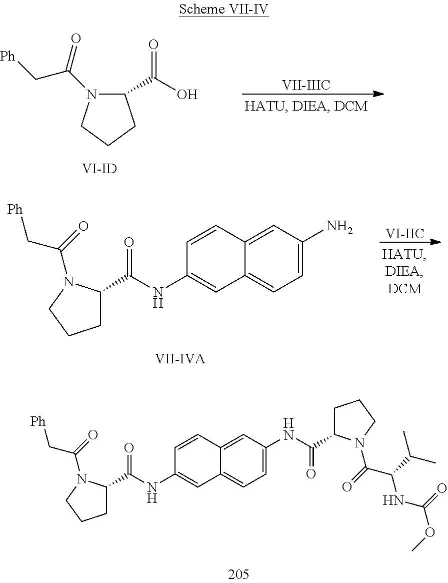

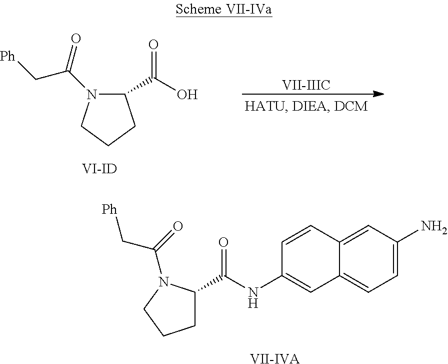

### General Procedure VII-N

To a solution of naphthalene-2,6-diamine (VII-IIIC) (100 mg, 0.633 mmol) in anhydrous CH₂Cl₂ (8 mL) was added compound VI-ID (590 mg, 2.532 mmol), HATU (312 mg, 0.823 mmol) and DIEA (245 mg, 1.9 mmol). The mixture was stirred at r.t. overnight. The mixture was diluted with CH₂Cl₂ (50 mL), washed with 5% citric acid (5 mL×2), water (5 mL×2) and brine (5 mL×2). The organic layer was dried over anhydrous Na₂SO₄, and concentrated in vacuo. The residue was purified by prep-TLC (PE/EA=1:2) to give compound VII-IVA (50 mg, yield 22%).

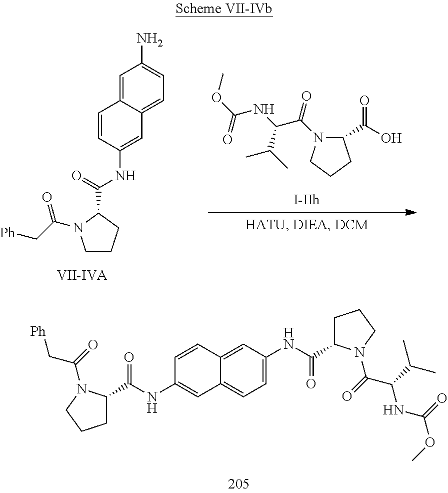

### General Procedure VII-O

To a solution of compound VII-IVA (50 mg, 0.134 mmol) and compound I-IIh (69 mg, 0.254 mmol) in anhydrous CH₂Cl₂ (8 mL) was added HATU (76 mg, 0.2 mmol) and DIEA (68 mg, 0.527 mmol). The reaction mixture was stirred at r.t. for 4.5 hrs. The mixture was diluted with CH₂Cl₂ (50 mL), washed with 5% citric acid (5 mL×2), water (5 mL×2) and brine (5 mL×2). The organic layer was dried over anhydrous Na₂SO₄, and concentrated in vacuo. The residue was purified by prep-HPLC to afford compound 205 (40 mg, yield 48%) as white solid. MS (ESI) m/z (M+H)⁺ 628.2.

### Example VII-V

### Preparation of Compound 206, 207, 208, and 209

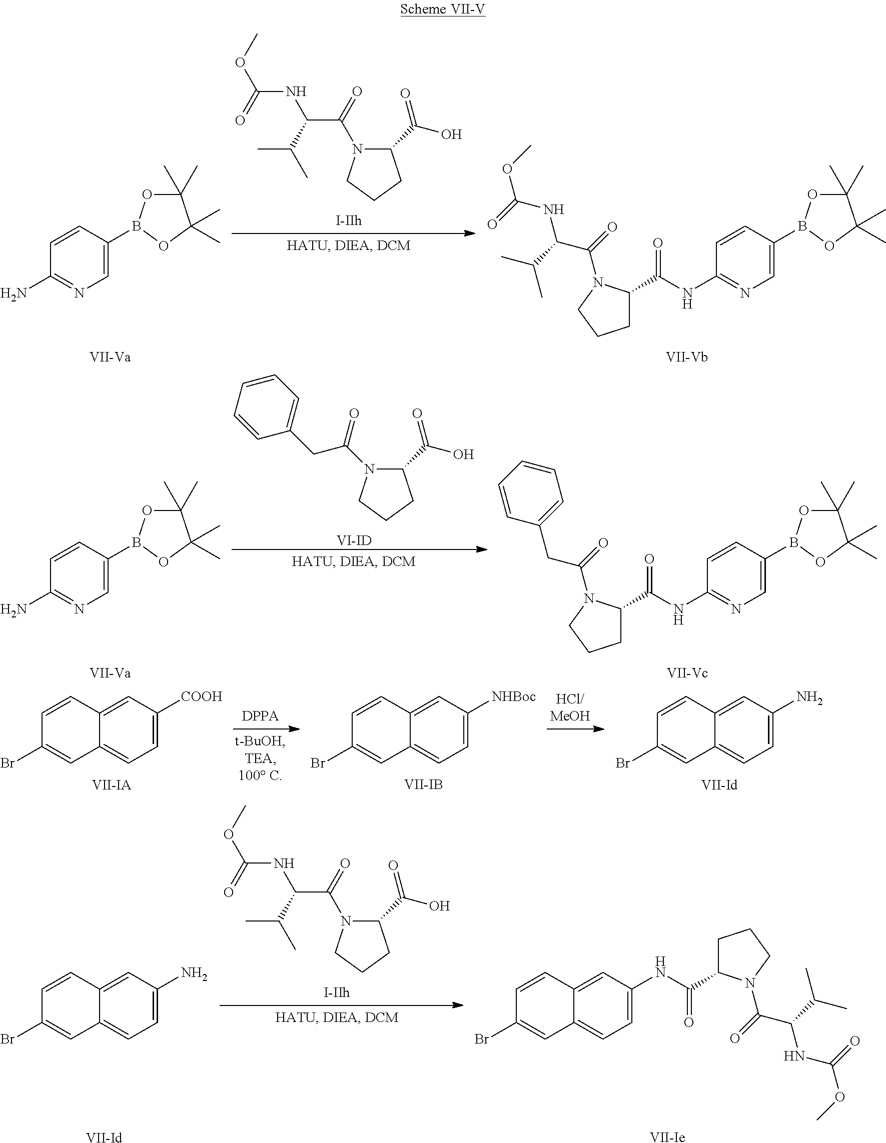

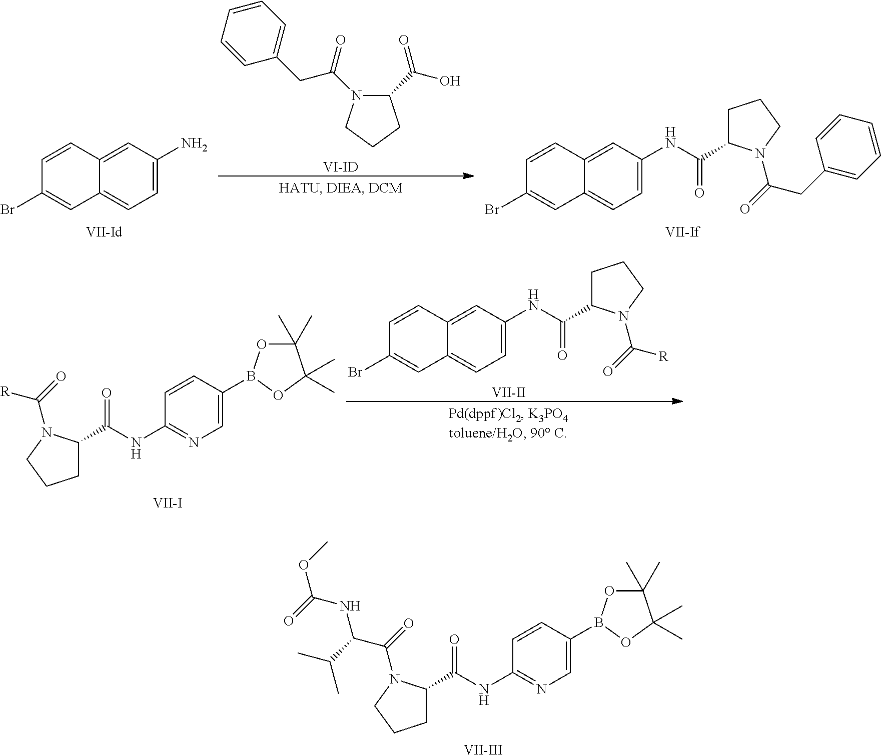

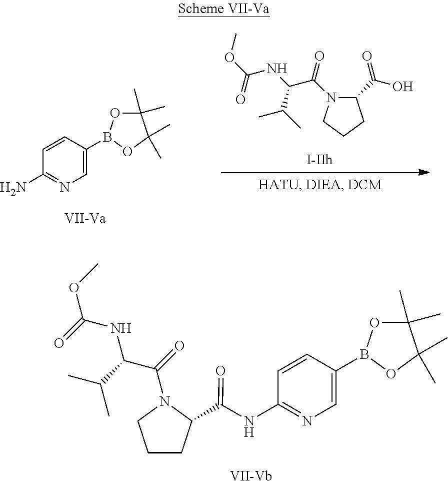

### General Procedure VII-P

A flask was charged with 5-(4,4,5,5-tetramethyl-1,3,2-dioxaborolan-2-yl)pyridin-2-amine (VII-Va) (296 mg, 1.34 mmol), I-IIh (439 mg, 1.61 mmol), HATU (988 mg, 2.6 mmol), DCM (15 mL) and DIEA (691 mg, 5.35 mmol). The resulting mixture was stirred at room temperature overnight. After the material was consumed, the mixture was diluted with EtOAc (50 mL), washed with brine, dried over anhydrous Na₂SO₄, and concentrated in vacuo to afford compound VII-Vb (480 mg, yield 74%).

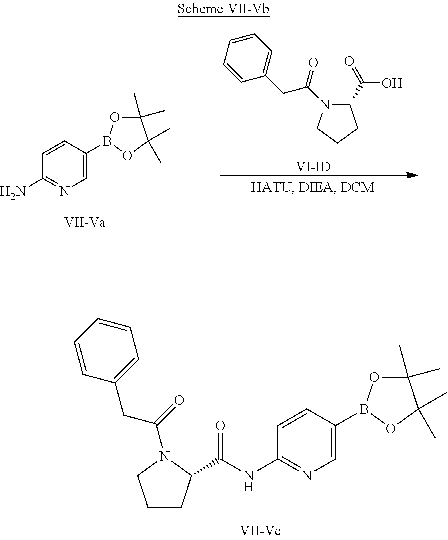

### General Procedure VII-Q

Compound VII-Vc is prepared in the same manner as the preparation of compound VII-Vb (440 mg, yield 63%).

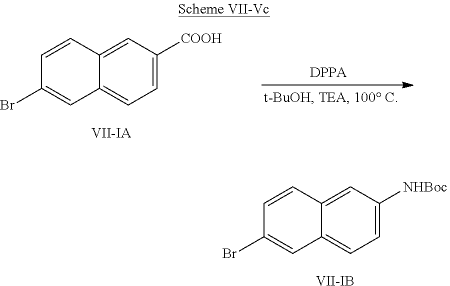

### General Procedure VII-R

To a solution of 6-bromonaphthalene-2-carboxylic acid (VII-IA) (11.5 g, 45.8 mmol) in t-BuOH (50 mL) was added triethylamine (4.86 g, 48.1 mmol) and DPPA (13.2 g, 48.1 mmol). The reaction mixture was stirred at 100° C. for 5 hrs. The mixture was concentrated, washed by water and neutralized by saturated aq. NaHCO₃, the solid was filtered to afford compound VII-IB (12 g, yield 85%).

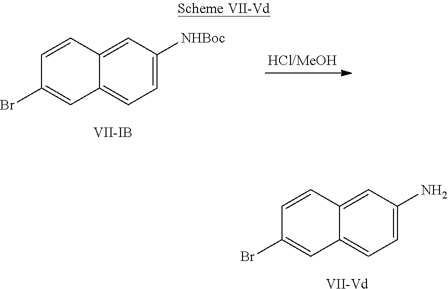

### General Procedure VII-S

A flask was charged with VII-IB (5.5 g, 17.1 mmol) and HCl/MeOH (4M, 170 mL) was stirred at room temperature for 4 hrs. After the completion of reaction, the mixture was concentrated to afford 6-bromonaphthalen-2-amine (VII-Vd) (2.5 g, yield 66%).

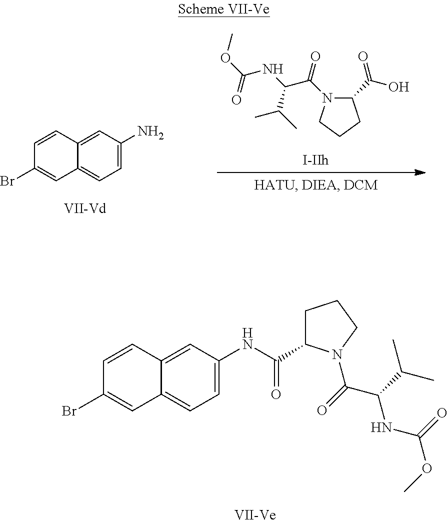

### General Procedure VII-T

A flask was charged with 6-bromonaphthalen-2-amine (VII-Vd) (406 mg, 1.84 mmol), I-IIh (500 mg, 1.84 mmol), HATU (1.19 g, 3.12 mmol), DCM (15 mL) and DIEA (949 mg, 7.36 mmol) was stirred at room temperature overnight. After the material was consumed, the mixture was diluted with EtOAc (100 mL), washed with brine, dried over anhydrous Na₂SO₄, and concentrated in vacuo to afford compound VII-Ve (600 mg, yield 68%).

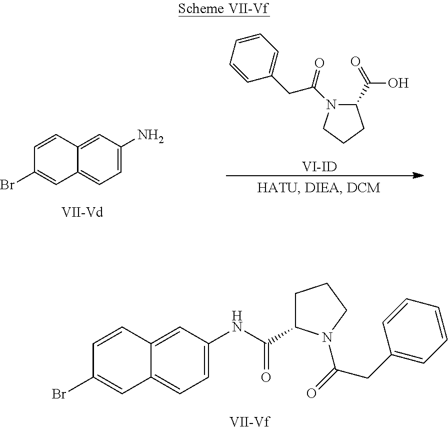

### General Procedure VII-U

Compound VII-Vf is prepared in the same manner as the preparation of compound VII-Ve (300 mg, yield 86%).

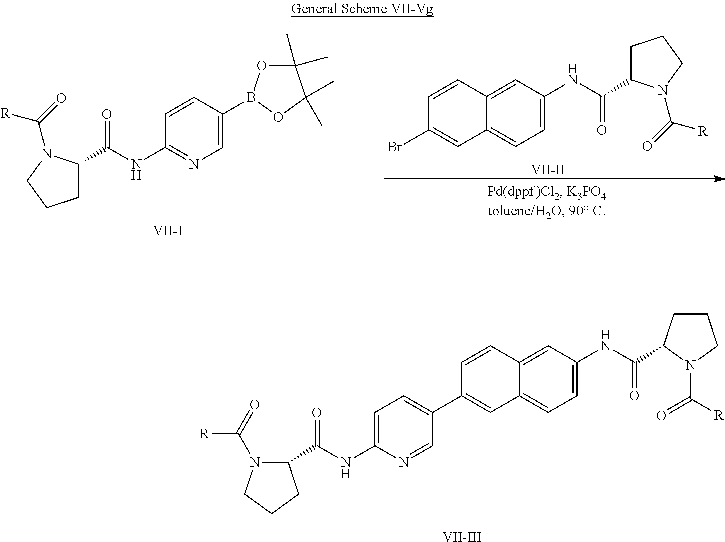

### General Method VII-V

A flask is charged with VII-I (1 eq.), VII-II (1 eq.), Pd(dppf)Cl₂ (0.1 eq.), K₃PO₄ (2 eq.), toluene (2 mL) and water (1 mL). The flask is purged with nitrogen and stirred at 90° C. overnight under nitrogen. The mixture is poured into water, neutralized and extracted with EtOAc, the combined extracts are washed with brine, dried over anhydrous Na₂SO₄, and concentrated in vacuo to afford a residue. The residue is purified by prep-HPLC to afford the compound.

General Method VII-V was followed for preparation of the following compounds:

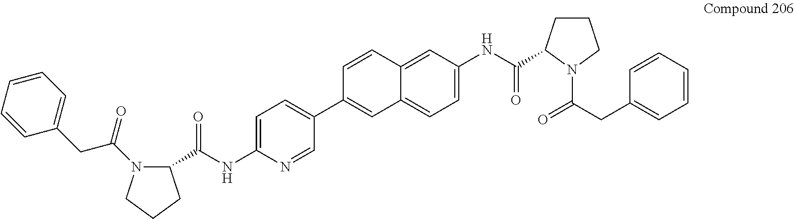

10 mg, 5%. MS (ESI) m/z (M+H)⁺ 665.7.

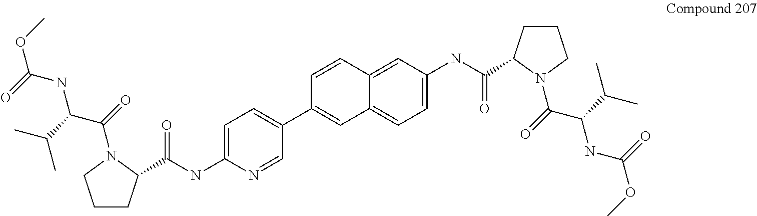

80 mg, 18%. MS (ESI) m/z (M+H)⁺ 744.3

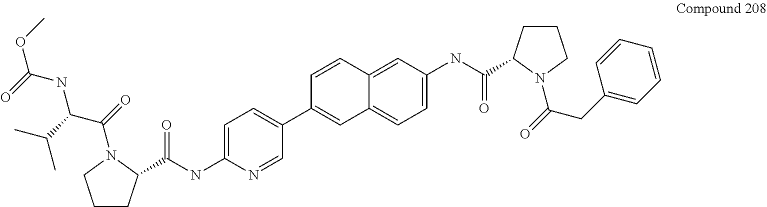

10 mg, 7%. MS (ESI) m/z (M+H)⁺ 705.3

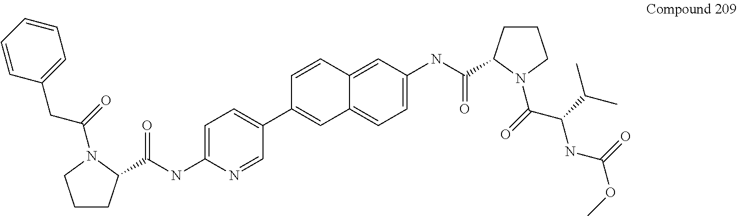

23 mg, 10%. MS (ESI) m/z (M+H)⁺ 705.3

### Example VII-VI

### Preparation of Compound 210

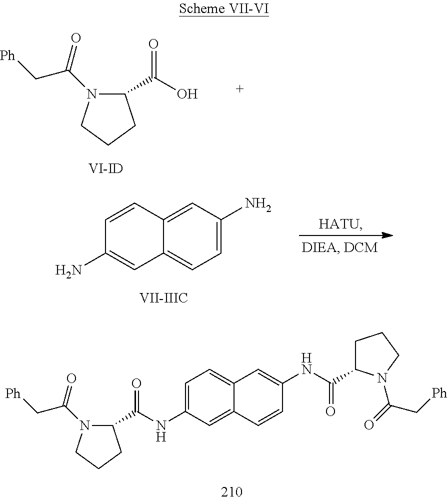

### General Procedure VII-W

To a solution of compound VI-ID (590 mg, 2.532 mmol) and naphthalene-2,6-diamine (VII-IIIC) (100 mg, 0.633 mmol) in anhydrous dichloromethane (10 mL) was added HATU (624 mg, 1.646 mmol) and DIEA (326 mg, 2.532 mmol). The reaction solution was stirred at r.t overnight. The mixture was quenched by water and extracted with EtOAc (15 mL×3). The combined organic layers were dried over anhydrous Na₂SO₄ and concentrated. The residue was purified by prep-HPLC to afford compound 210 (60 mg, 16% yield) as pale-yellow solid. MS (ESI) m/z (M+H)⁺ 589.3.

### Example VII-VII

### Preparation of Compound 211

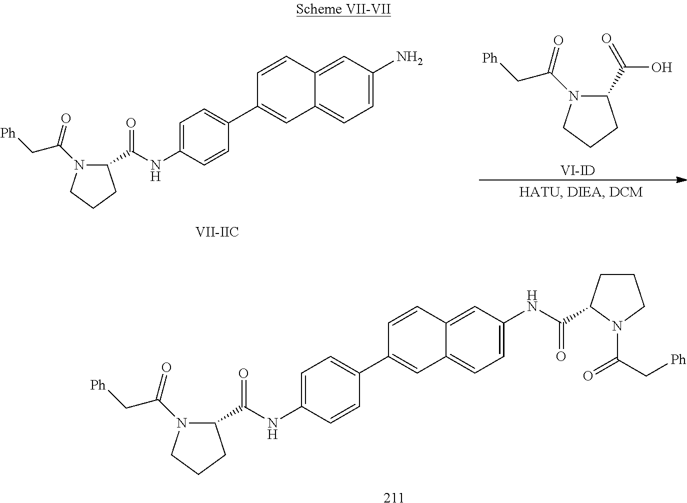

### General Procedure VII-X

A flask was charged with compound VII-IIC (50 mg, 0.11 mmol), compound VI-ID (39 mg, 0.17 mmol), HATU (84 mg, 0.22 mmol) and DIEA (57 mg, 0.44 mmol), anhydrous CH₂Cl₂ (10 mL). Then the mixture was stirred at room temperature for 16 hours. The LCMS showed the reaction was completed. The mixture was concentrated, purified by prep-HPLC to afford compound 211 as a light yellow solid. (30 mg, yield 41%). MS (ESI) m/z (M+H)⁺ 655.3.

### Example VII-VIII

### Preparation of Compound 212 and 213

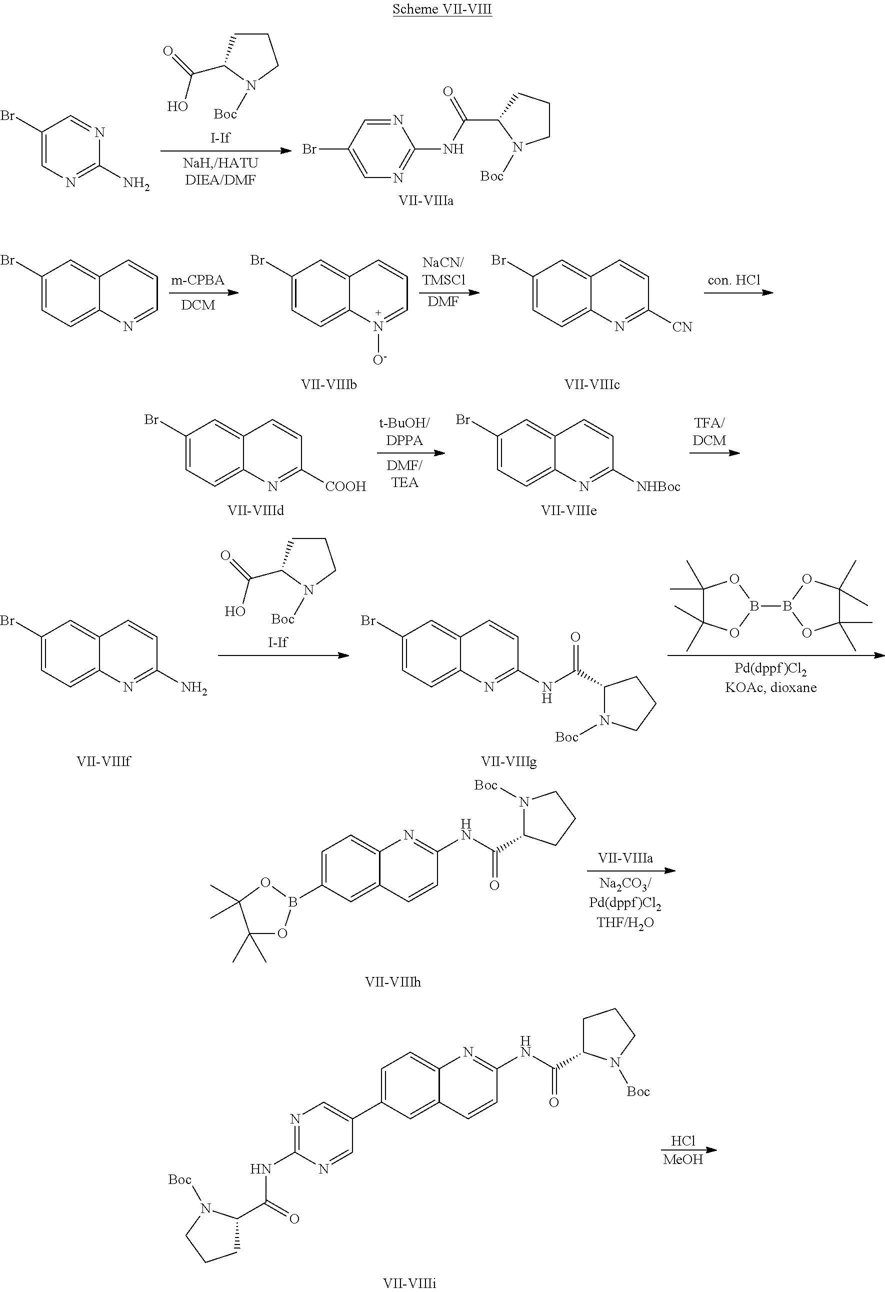

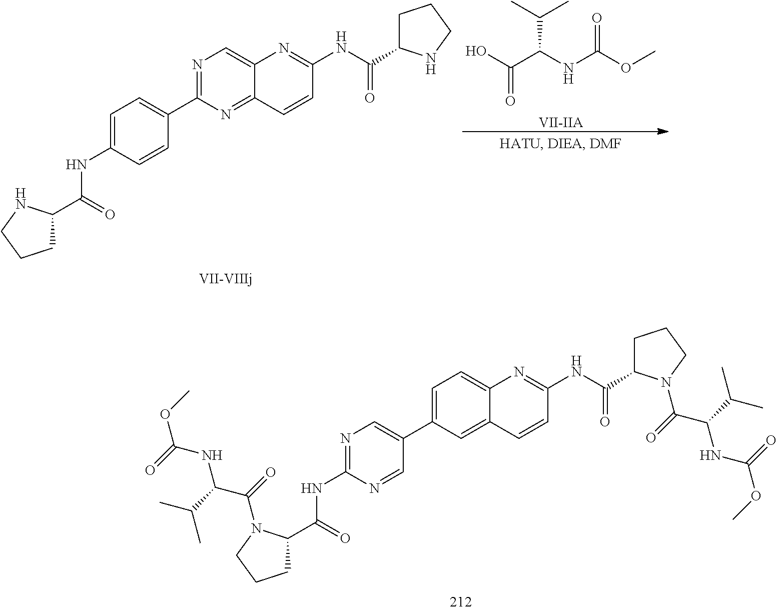

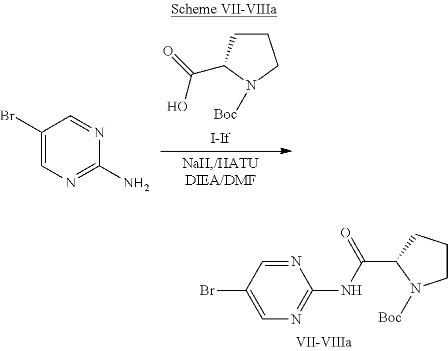

### General Procedure VII-Y

To a solution of compound I-If (0.5, 2.32 mmol) in DMF (10 mL) were added HATU (1.06 g, 2.79 mmol) and DIEA (0.6 g, 4.65 mmol). The mixture was stirred for 1 hour at room temperature. And then added to a solution of 2-amino-5-bromopyrimidine (0.4 g, 2.32 mmol) and NaH (0.067 g, 2.79 mmol, 60 percent) in 10 mL DMF previously stirred for 30 min. at −20° C. Then the reaction mixture was allowed to warm to room temperature and stirred overnight. After diluted with 50 mL DCM and was quenched with 30 mL water. The mixture was extracted with DCM (3×50 mL). Combined organic phase extracts was dried over Na₂SO₄. Then concentrated organic layer and the residue was purified by column chromatography to give compound VII-VIIIa (0.2 g, yield 23%). ¹H NMR (300 MHz, CDCl₃) δ10.09 (s, 1H), 8.79-8.61 (s, 2H), 4.53 (s, 1H), 3.72-3.44 (s, 1H), 2.02-1.91 (m, 4H), 1.50-1.45 (s, 9H), MS (ESI) m/z (M+Na)⁺ 394.8.

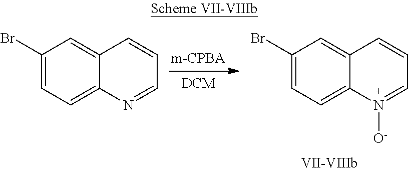

### General Procedure VII-Z

A solution of 6-bromoquinoline (40 g, 0.192 mol) in dry DCM (500 mL) was added m-CPBA (48.2 g 0.23 mol) in one portion under ice cooling with stirring. The reaction was allowed to warm to ambient temperature and stirred at this temperature for one hour. Then the mixture was washed with Na₂CO₃ solution (1.2 eq). And the organic layer extracts was separated dried over Na₂SO₄ and concentrated under reduce pressure to give compound VII-VIIIb (30 g yield 70%) which was used for next step without further purification.

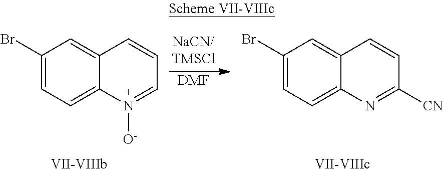

### General Procedure VII-AA

To a mixture of compound VII-VIIIb (2 g, 8.93 mmol), NaCN (0.875 g, 17.86 mmol), and TEA (7.42 mL, 53.6 mmol) in absolute DMF (60 mL) with stirring was added TMSCl (5.66 mL, 44.65 mmol) within 40 min. Then the temperature was rinsed to 100° C. and stirred overnight at this temperature. The mixture was cooled to room temperature before it was filtrated. The filtrate was evaporated and the residue was purified with column chromatography on silica gel (petroleum ether:EtOAc=10:1) to give compound VII-VIIIc. (1.2 g, yield: 60%). ¹H NMR (300 MHz, DMSO-d₆) δ 8.19-7.92 (d, 1H), 7.84-7.83 (s, 2H), 7.66-7.63 (d, 2H), 7.51-7.48 (d, 2H), MS (ESI) m/z (M+H)⁺ 232.8.

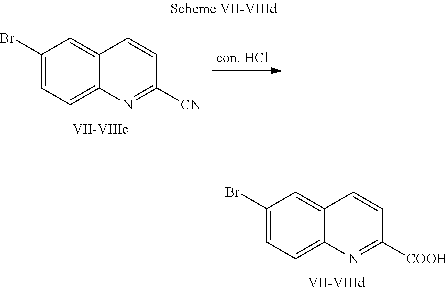

### General Procedure VII-AB

Compound VII-VIIIc (1 g, 0.3 mmol) was dissolved in concentrated hydrochloric acid aqueous (40 mL). The solution was stirred and heated to reflux for 19 h. After the mixture was cooled to room temperature, the precipitate was collected by filtration, and was washing with water, to give compound VII-VIIId (0.6 g, yield: 46%). MS (ESI) m/z (M+H)⁺ 253.9.

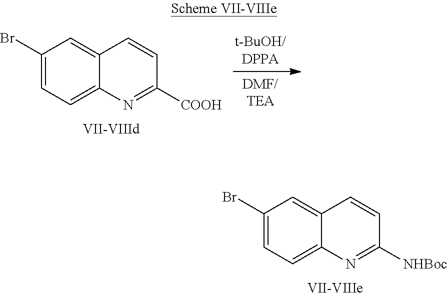

### General Procedure VII-AC

A mixture of compound VII-VIIId (0.6 g, 2.37 mmol), t-BuOH (12 mL, 0.125 mmol), DPPA (0.53 mL, 2.46 mmol) and TEA (0.65 mL, 4.67 mmol) was dissolved in 16 mL DMF. The mixture was heated to 100° C. and stirred for 7 h. Then the mixture was allowed to cool to room temperature. Evaporation gave a black oil which was purified by column chromatography on silica gel (petroleum ether:EtOAc=20:1) to give compound VII-VIIIe (0.35 g, yield 45%). ¹H NMR (300 MHz, DMSO-d₆) δ10.14 (s, 1H), 8.20-8.17 (d, 1H), 8.07-8.06 (s, 1H), 8.00-7.97 (d, 1H), 7.70-7.69 (d, 1H), 7.67-7.66 (d, 1H), 7.60-7.57 (d, 1H), 1.40 (s, 9H), MS (ESI) m/z (M+H)⁺ 323.

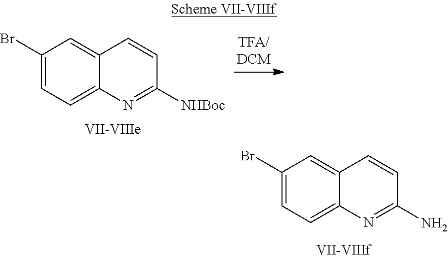

### General Procedure VII-AD

Compound VII-VIIIe (300 mg, 0.93 mmol) was dissolved in DCM (10 mL) and TFA (10 mL). Then the mixture was stirred for 5 h at room temperature. After this the mixture was concentrated under reduced pressure to give 6-bromoquinolin-2-amine (VII-VIIIf, 200 mg, yield 97%), which was used for next step without further purification.

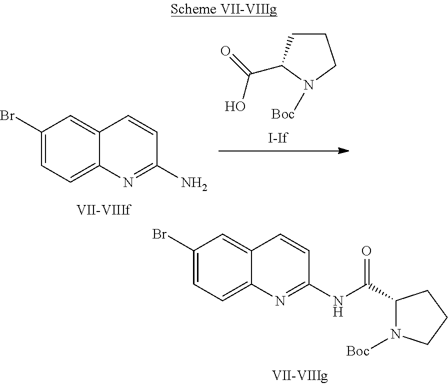

### General Procedure VII-AE

To a solution of compound I-If (0.3 g 1.34 mmol) and HATU (0.56 g 1.34 mmol) in dry DMF (15 mL) was added DMA (0.35 g, 2.68 mmol). The mixture was stirred for 1 h at room temperature. Then 6-bromoquinolin-2-amine (VII-VIIIf, 0.288 g, 1.34 mmol) was added into the mixture. The reaction mixture was stirred overnight at room temperature, and quenched with saturate NH₄Cl solution then extracted with EA (4×30 mL). Combined organic layer extracts was dried over Na₂SO₄ and concentrated under reduced pressure to give the crude product which was purified by chromatography on silica gel column to give compound VII-VIIIg (200 mg, yield: 64%). ¹H NMR (300 MHz, DMSO-d₆): δ8.45-8.45 (d, 1H), 8.44-8.44 (d, 1H), 8.33-8.29 (q, 1H), 7.32-7.28 (q, 2H), 4.06-4.04 (t, 1H), 3.34-3.30 (t, 2H), 1.83-1.69 (m, 4H), 1.43-1.28 (s, 9H), MS (ESI) m/z (M+Na)⁺ 443.9.

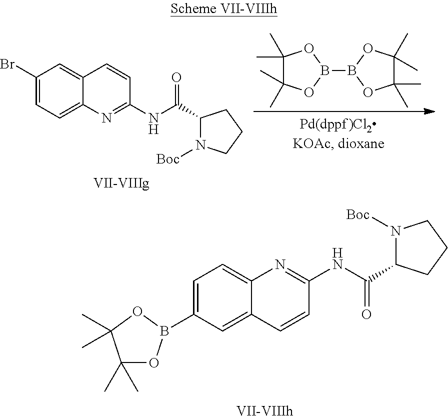

### General Procedure VII-AF

A mixture of compound VII-VIIIg (0.8 g, 1.9 mmol), bis(pinacolato)diboron (0.97 g, 3.8 mmol) Pd(dppf)Cl₂ (0.14 g 0.19 mmol) and KOAc (0.37 g, 3.8 mmol) was dissolved in 20 mL dioxane. The mixture was heated to reflux at 100-110° C. and stirred for 8 h at this temperature. Then it was concentrated and the residue purified by column chromatography to give compound VII-VIIIh (600 mg, yield 67.6%). ¹H NMR (300 MHz, DMSO-d₆) δ 8.42-8.38 (m, 1H), 8.35-825 (m, 2H), 7.84-7.83 (d, 1H), 7.74-7.71 (d, 1H), 4.45 (s, 1H), 1.90-1.80 (m, 2H), 1.29 (s, 12H), 1.18 (m, 4H), MS (ESI) m/z (M+H)⁺ 468.1.

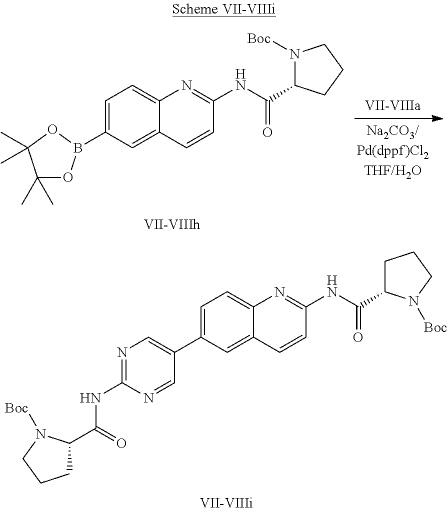

### General Procedure VII-AG

A mixture of compound VII-VIIIa (0.9 g, 2.43 mmol), compound VII-VIIIh (1.12 g, 2.43 mmol), Na₂CO₃ (0.52 g, 4.86 mmol) and Pd(dppf)Cl₂ (0.18 g, 0.024 mmol) was dissolved in 25 mL THF and 5 mL H₂O. The reaction mixture was heated to reflux at 80° C., and stirred overnight. Then the mixture was concentrated under reduced pressure and the residue was added water, extracted with EtOAc. Combined organic phase extracts was dried over Na₂SO₄. Then concentrated organic layer and the residue was purified by column chromatography to give compound VII-VIIIi (0.45 g, yield 30%), MS (ESI) m/z (M+H)⁺ 632.3.

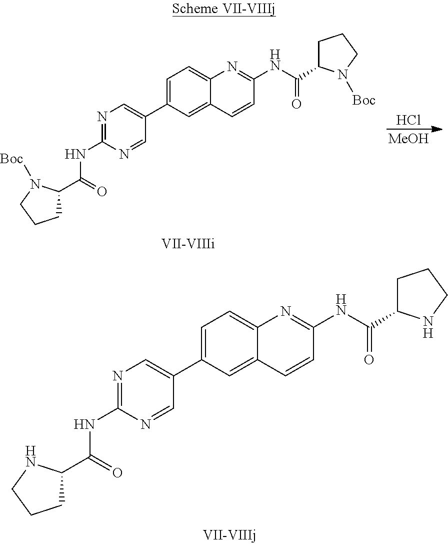

### General Procedure VII-AH

Compound VII-VIIIi (450 mg, 0.712 mmol) was dissolved in 40 mL HCl/MeOH. The mixture was stirred for 1.5 h at room temperature. Then concentrate the mixture under reduced pressure to give compound VII-VIIIj, which was used for next step without further purification.

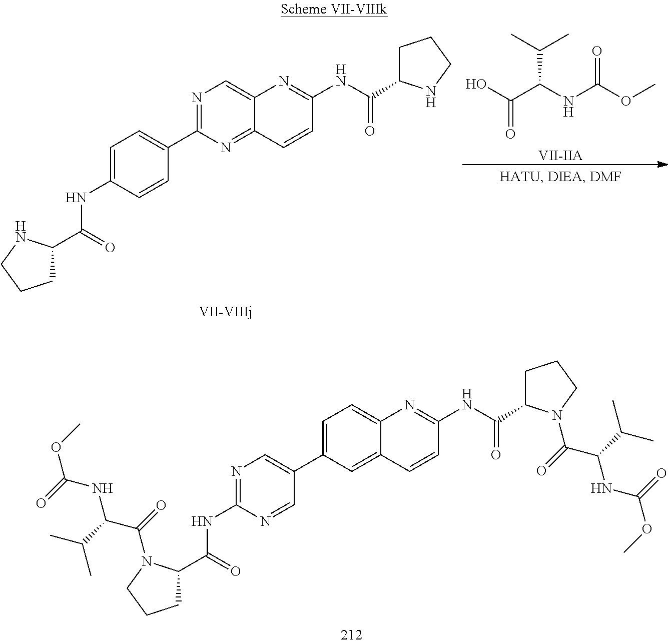

### General Procedure VII-AI

To a mixture of compound VI-IIA (95 mg, 0.35 mmol) and HATU (700 mg, 1.86 mmol) in 8 mL DFM was added DIEA (155 mg, 1.2 mmol). The mixture was stirred for 30 min at room temperature. Then it was added compound VII-VIIIj (200 mg, 0.465 mmol), and the reaction mixture was stirred overnight at room temperature. After this it was added 10 mL water and 30 mL EtOAc, and was extracted with EtOAc (30 mL×4). Combined organic phase extracts was dried over Na₂SO₄. Then concentrated organic layer and the residue was purified by prep-HPLC to afford compound 212 (60 mg, yield: 17.3%). ¹H NMR (400 MHz, DMSO-d₆): δ11.08 (s, 1H), 10.93 (s, 1H), 9.19 (s, 2H), 8.50-8.45 (m, 1H), 8.42-8.35 (m, 2H), 8.21-8.19 (m, 1H), 7.98-7.94 (m, 1H), 7.49-7.41 (m, 2H), 4.85-4.65 (s, 2H), 4.12-4.05 (m, 2H), 3.95-3.85 (m, 2H), 3.72-3.65 (m, 2H), 3.60-3.54 (m, 7H), 2.32-2.20 (m, 2H), 2.19-2.12 (m, 6H), 2.05-1.90 (m, 8H), 1.08-1.02 (m, 6H), 1.01-0.90 (m, 6H), MS (ESI) m/z (M+H)⁺ 746.2.

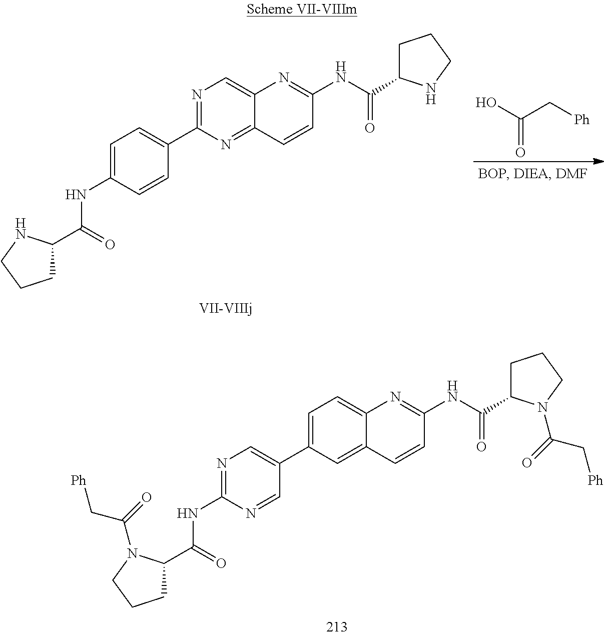

### General Procedure VII-AJ

To a mixture of compound VII-VIIIj (200 mg, 0.46 mmol) and 2-phenylacetic acid (152 mg 1.12 mmol) in 8 mL DMF was added DMA (480 mg, 3.7 mmol). The mixture was stirred for 30 min at room temperature. Then it was added BOP (617 mg, 1.4 mmol), and the reaction mixture was stirred overnight at room temperature. After this it was added 10 mL water and 30 mL EtOAc, and was extracted with EtOAc (30 mL×5). Combined organic phase extracts was dried over Na₂SO4. Then concentrated organic layer and the residue was purified by prep-HPLC to afford compound 213 (50 mg, yield: 16%). ¹H NMR (400 MHz, DMSO-d₆) δ11.26 (s, 1H), 11.14 (s, 1H), 9.17 (m, 2H), 8.41-8.29 (m, 3H), 8.14-8.11 (m, 1H), 7.93 (m, 1H), 7.18 (m, 10H) 4.76-4.65 (m, 2H), 3.75-3.68 (m, 4H), 3.59-3.55 (m, 4H), 2.20-2.14 (m, 2H), 2.04-1.83 (m, 6H), MS (ESI) m/z (M+H)⁺ 668.1.

### Example VII-IX

### Preparation of Compound 214

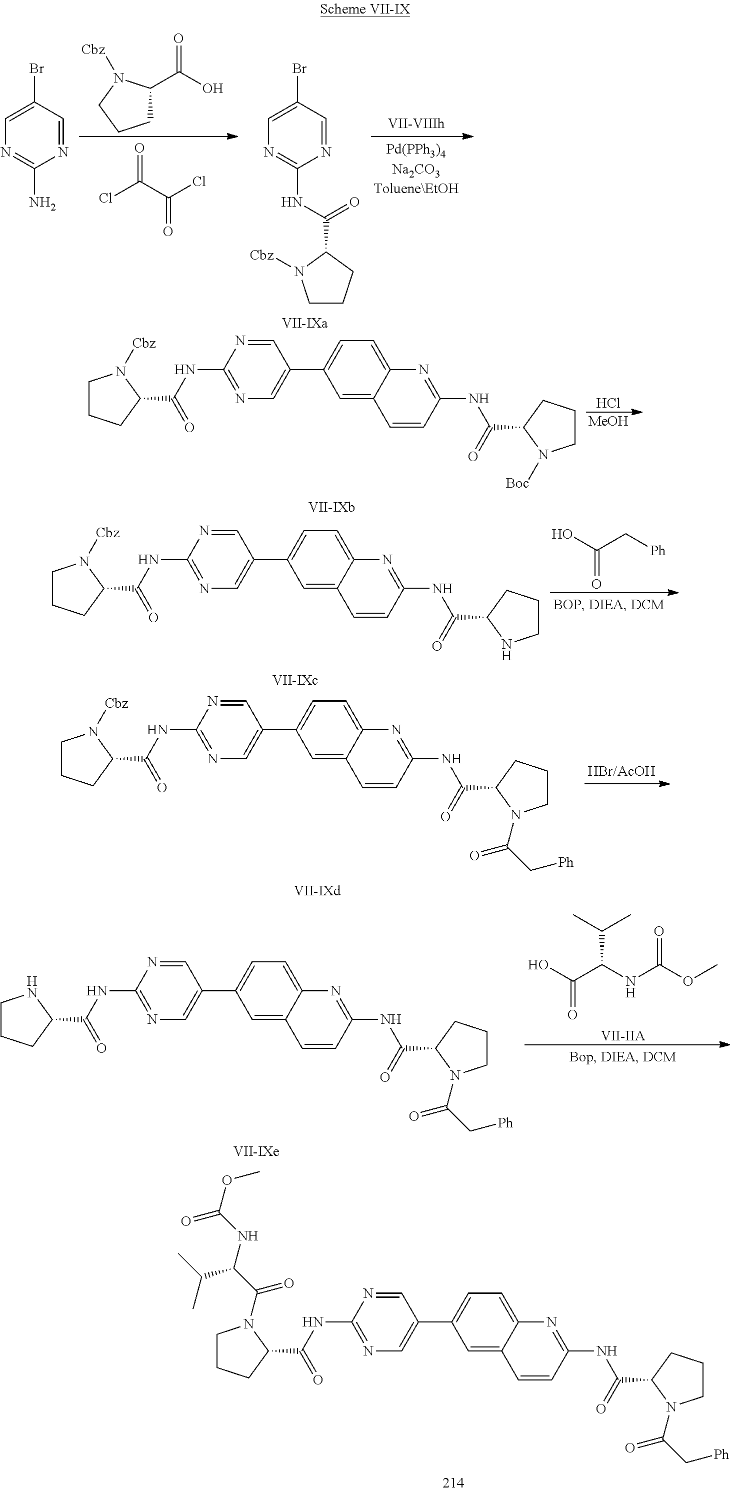

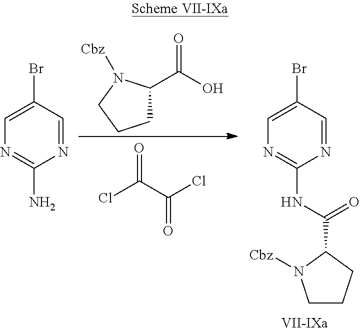

### General Procedure VII-JK

A flask was charged with Cbz-N-proline (6.37 g, 25.6 mmol), oxalyl dichloride (6.35 g, 50 mmol), DCM (40 mL) and one drop of DMF was stirred at room temperature for 1.5 h. The mixture was concentrated and then dissolved in DMF, treated with and NaH (1.02 g, 25.6 mmol), the mixture was stirred at 0° C. for 1 h. Then a solution of 2-amino-5-bromopyrimidine (4 g, 23.2 mmol) in DMF was added and stirred at room temperature overnight. The mixture was poured into water and neutralized. Filtered and the organic layer was extracted for 2 times and concentrated. Purified by chromatography on silica gel to give compound VII-IXa (2 g, yield: 21.5%).

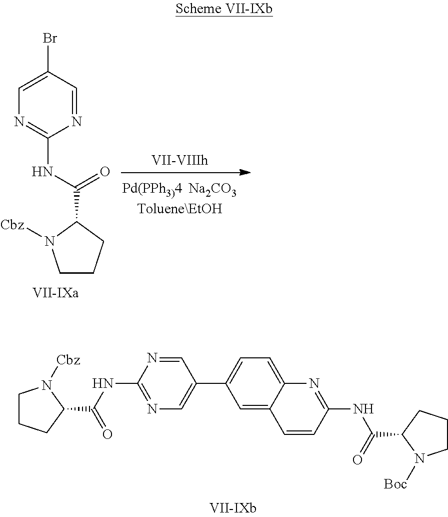

### General Procedure VII-JL

To a solution of compound VII-IXa (700 mg, 1.499 mmol) in toluene/EtOH (3 mL) was added compound VII-VIIh (605 mg, 1.499 mmol), Na₂CO₃ and Pd(PPh₃)₄ (49 mg, catalyzed amount). The mixture was charged with N₂ for 5 minutes and heated to 80° C. overnight. LCMS detected that the reaction was completed. The mixture was diluted with water (100 mL) and extracted with EtOAc (150 mL×3). The combined organic layers were concentrated and the residue was purified by column chromatograph on silica gel (eluting with PE:EtOAc=10:1 to 2:1) to give compound VII-IXb (700 mg, yield: 70%) as a white solid. MS (ESI) m/z (M+H)⁺ 666.

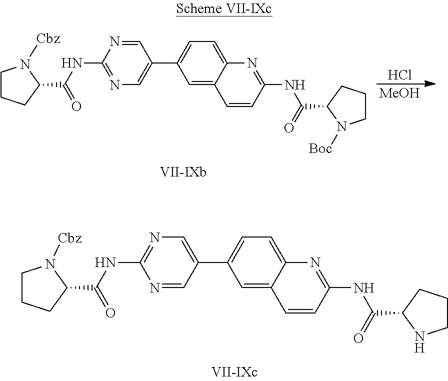

### General Procedure VII-JM

Compound VII-IXb (160 mg, 0.24 mmol) was dissolved in 20 mL HCl/MeOH. The mixture was stirred for 2 h at room temperature. Then concentrate the mixture under reduced pressure to give compound VII-IXc, which was used for next step without further purification.

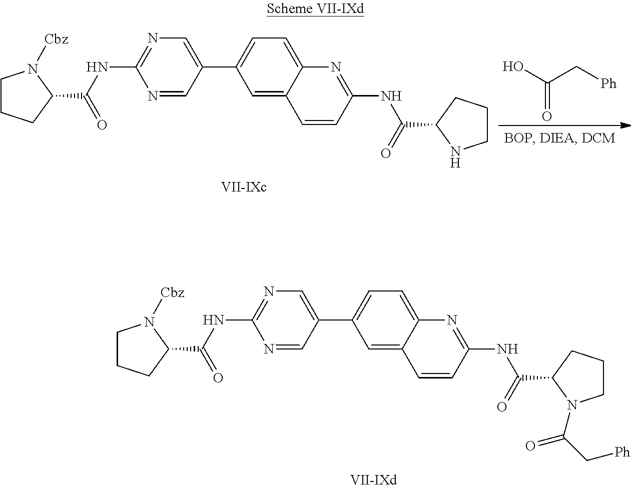

### General Procedure VII-JN

To a mixture of compound VII-IXc (150 mg, 0.22 mmol) and phenylacetic acid (36.8 mg, 0.27 mmol) in 20 mL DCM was added DIEA (116 mg, 0.9 mmol). The mixture was stirred for 30 min at room temperature. Then it was added BOP (120 mg, 0.27 mmol), and the reaction mixture was stirred overnight at room temperature. Then the mixture was purified directly by Prep. TLC (DCM:MeOH=10:1) to give compound VII-IXd (60 mg, yield: 19%). MS (ESI) m/z (M+H)⁺ 684.1.

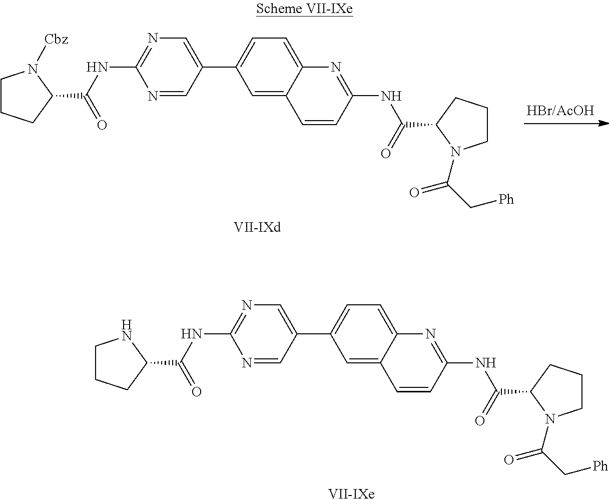

### General Procedure VII-JO

A mixture of compound VII-IXd (120 mg, 0.18 mmol) and 5 mL HBr solution (in AcOH) was dissolved in 10 mL AcOH. The reaction mixture was stirred for 5 h at room temperature. Then it was poured into ice-water (100 mL) and adjusted to pH 8 by progressively adding solid Na₂CO₃. Then it was extracted with DCM (200 ml×3). Combined organic layer extracts was washed by water and dried over Na₂SO₄. Concentrate the organic phases and residue was purified by TLC preparation chromatography (DCM:MeOH=10:1) to give compound VII-IXe (30 mg, yield: 31%). MS (ESI) m/z (M+H)⁺ 550.5.

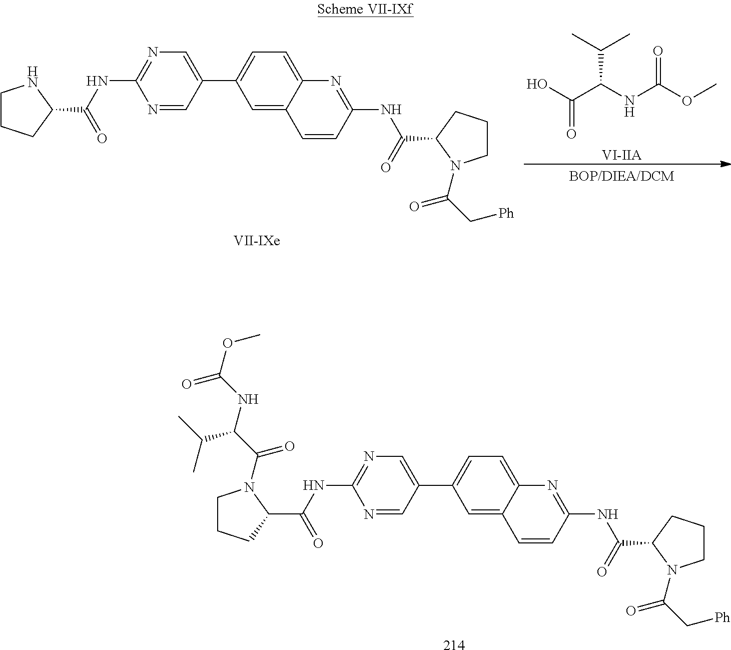

### General Procedure VII-JP

To a mixture of compound VII-IXe (50 mg, 0.09 mmol) and compound VI-IIA (16 mg, 0.09 mmol) in 15 mL DCM was added DIEA (60 mg, 0.36 mmol). The mixture was stirred for 30 min at room temperature. Then it was added BOP (50 mg, 0.11 mmol), and the reaction mixture was stirred overnight at room temperature. Then the mixture was added water and extracted with DCM (50 mL×3). Combined organic layer extracts was dried over Na₂SO₄. Concentrate the organic phases and residue was purified by HPLC preparation chromatography to give compound 214 (10 mg, yield 15%). ¹H NMR (400 MHz, CDCl₃) δ10.03-9.62 (s, 2H), 9.04-8.89 (s, 2H), 8.48 (d, 1H), 8.18 (d, 1H), 7.99-7.99 (d, 1 H), 7.86-7.84 (s, 1H), 7.75-7.65 (d 1H) 7.33 (m, 5H), 5.36-5.32 (d, 1H), 5.23 (s 1H), 4.69 (d, 1H), 4.22 (t, 1H), 3.89 (s, 3H), 3.67 (m, 5H), 3.42-3.30 (m, 1H), 2.26-2.21 (m, 2H), 1.80 (m, 1H), 1.59-1.45 (m, 6H), 0.82-0.71 (s, 3H), 0.65-0.58 (s, 3H), MS (ESI) m/z (M+H)⁺ 707.3.

### Example VII-X

### Preparation of Compound 215

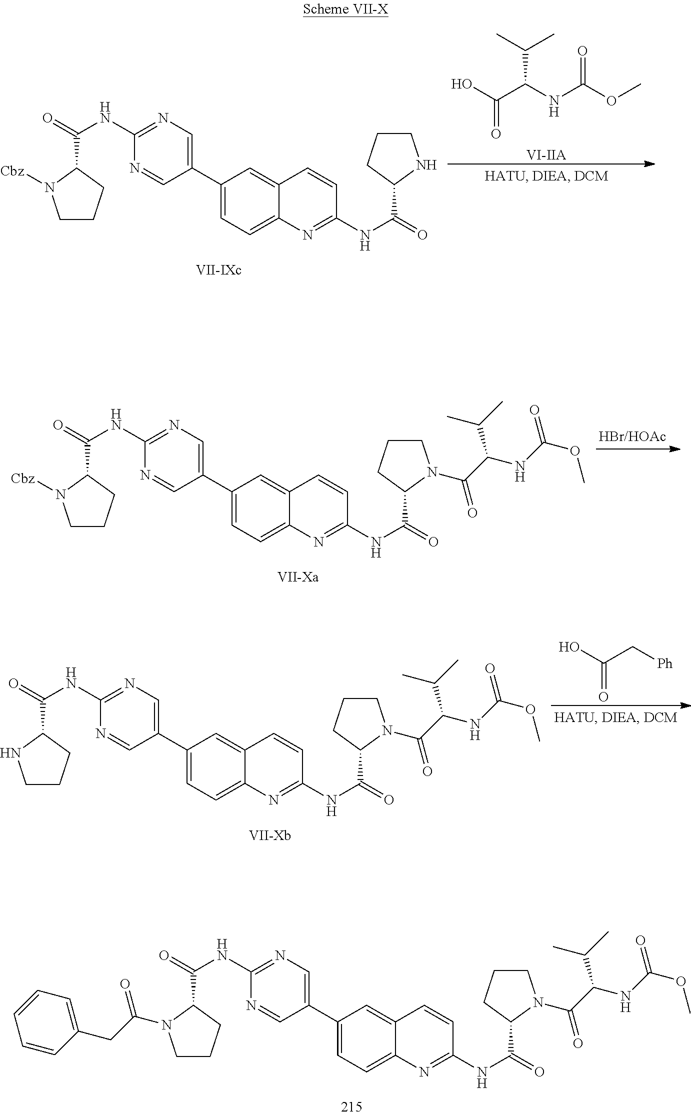

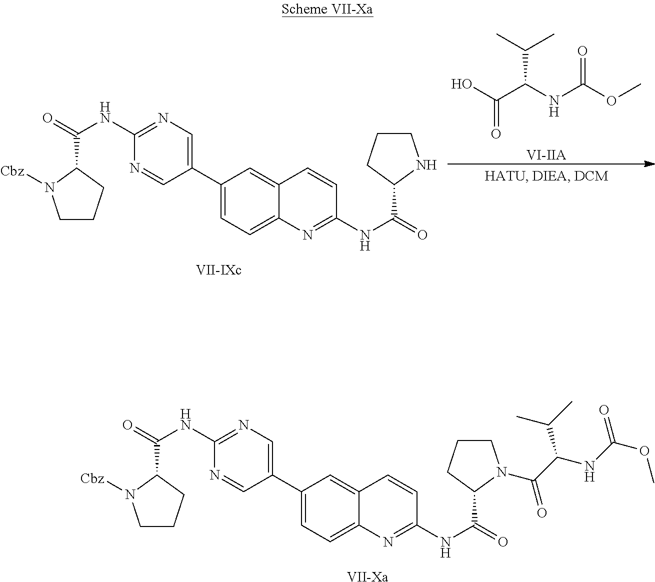

### General Procedure VII-AQ

To s solution of compound VII-IXc (126 mg, 0.223 mmol) in anhydrous DCM (2 mL) was added compound VI-IIA (39 mg, 0.223 mmol), HATU (169 mg, 0.445 mmol) and DIPEA (115 mg, 0.89 mmol). The reaction solution was stirred at r.t. for 4 hours. The mixture was diluted with water (10 mL) and extracted with EtOAc (5 mL×3). The combined organic layers were concentrated and the residue was purified to give VII-Xa (120 mg, 74%) as a yellow solid. MS (ESI) m/z (M+H)⁺ 722.

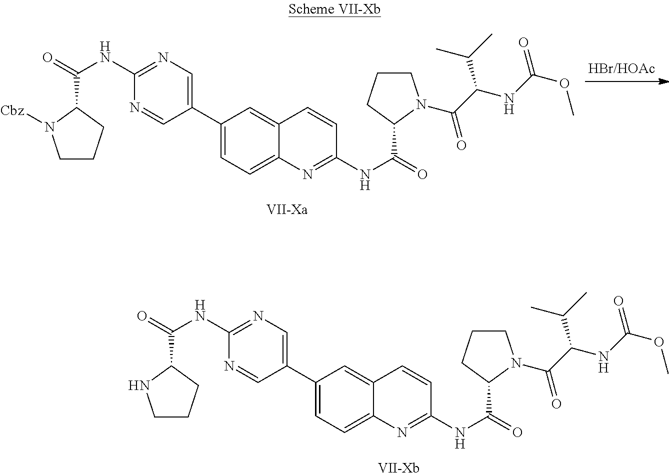

### General Procedure VII-AR

A solution of compound VII-Xa (120 mg, 0.166 mmol) in AcOH (0.03 mL) was added HBr/AcOH (0.35 mL) and the mixture was stirred at r.t. overnight. LCMS detected the reaction was completed. The reaction solution was concentrated under reduced pressure to afford compound VII-Xb (80 mg, 82%). MS (ESI) m/z (M+H)⁺ 588.

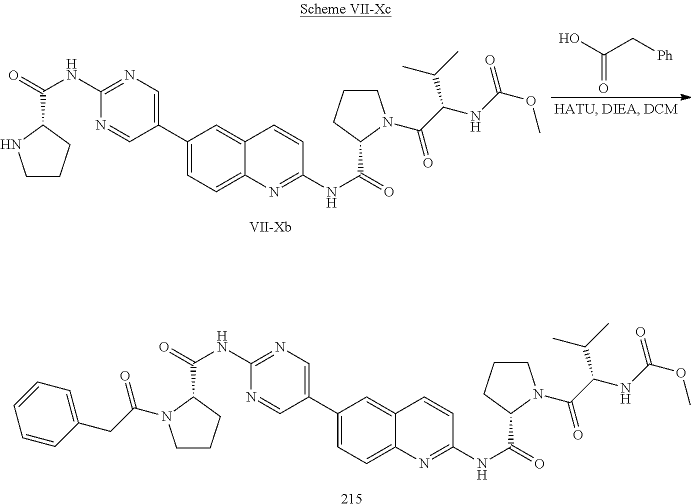

### General Procedure VII-AS

To s solution of compound VII-Xb (98 mg, 0.167 mmol) in anhydrous DCM (2 mL) was added phenylacetic acid (27 mg, 0.200 mmol), HATU (127 mg, 0.334 mmol) and DIPEA (172 mg, 1.336 mmol). The reaction solution was stirred at r.t. for 4 hours. The mixture was diluted with water (10 mL) and extracted with EtOAc (5 mL×3). The combined organic layers were concentrated and the residue was purified by Prep-HPLC to afford compound 215 (5 mg, 4%) as a white solid. ¹H NMR (400 MHz, CDCl₃) δ 10.251 (s, 1H), 9.537 (s, 1H), 8.901 (m, 2H), 8.391-8.368 (m, 1H), 8.200-8.178 (m, 1H), 7.951-7.929 (m, 1H), 7.877-7.802 (m, 1H), 7.797-7.775 (m, 1H), 7.309-7.240 (m, 3H), 5.495-5.497 (m, 1H), 4.974 (m, 1H), 4.836 (m, 1H), 4.387 (m, 1H), 4.370 (m, 3H), 3.871 (m, 4H), 3.775 (m, 1H), 3.505 (m, 1H), 3.481 (s, 1H), 2.613 (m, 1H), 2.562 (m, 2H), 2.577 (m, 4H), 1.663 (m, 3H), 1.279 (m, 1H), 1.033 (m, 3H), 0.891 (m, 3H).

### Example VII-XI

### Preparation of Compound 216 and 217

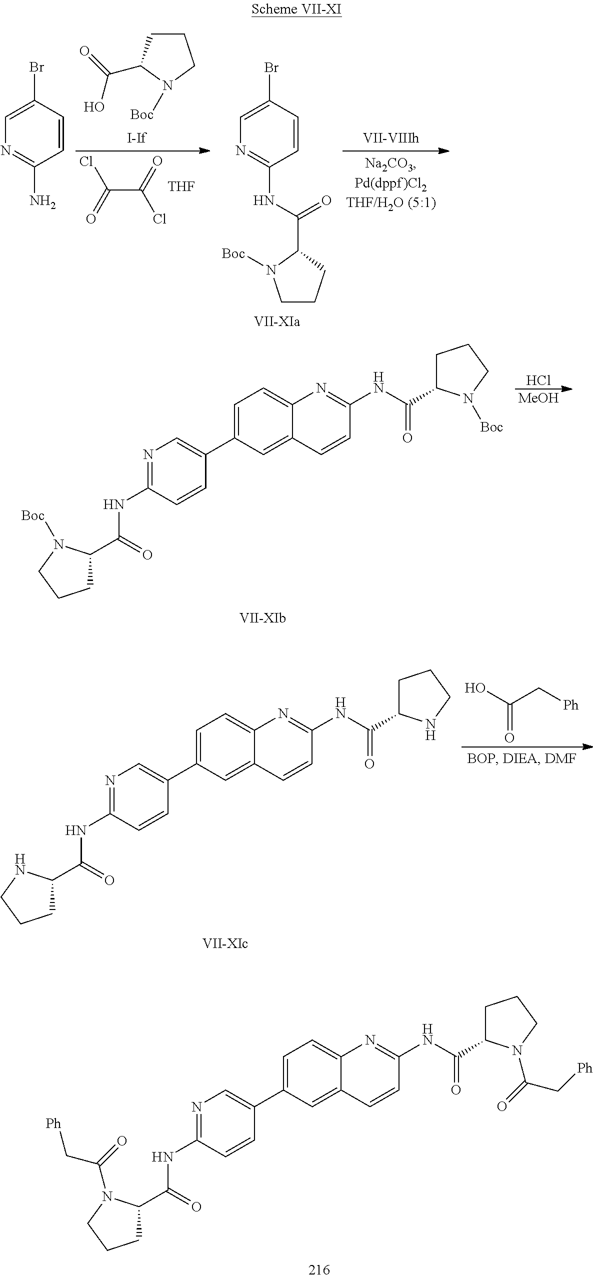

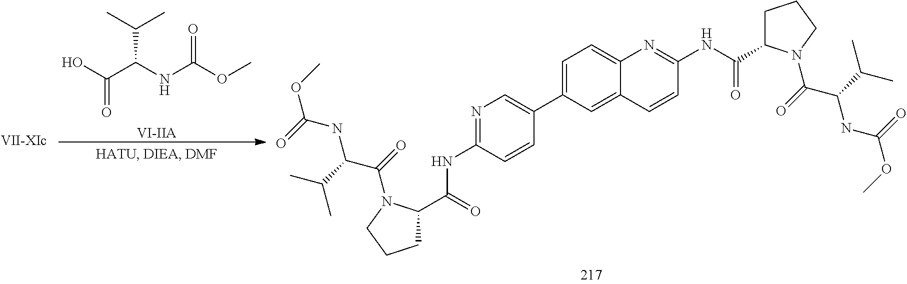

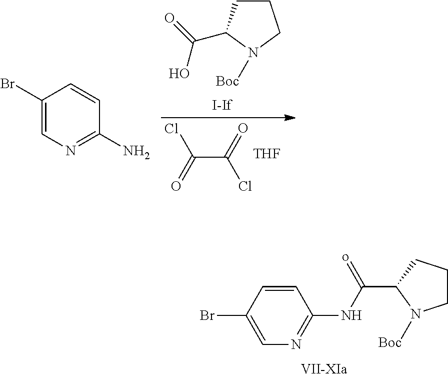

### General Procedure VII-AT

Compound I-If (1.97 g, 9.2 mmol) was dissolved in 150 mL THF under nitrogen cooled in ice water bath, added dry pyridine (3.7 mL, 45.8 mmol) followed by the dropwise addition of oxalyl chloride (2 mL, 22.9 mmol). A precipitate formed immediately. The reaction mixture was vigorous stirred at 0° C. for 2 h, then at ambient temperature for one hour. Subsequently 100 mL of THF was added, and the resulting mixture was passed through a filter. The solvent was removed and the remaining residue was dissolved in 100 mL of DCM under nitrogen, at 0° C., pyridine (3 mL) and 2-amino-5-bromopyridine (1.32 g, 9.2 mmol) was added. The reaction mixture was stirred at room temperature for 3 hours, and then the solvent was removed. The residue was purified by column chromatography to provide compound VII-XIa (450 mg, yield: 13%), MS (ESI) m/z (M+H)⁺ 370.0.

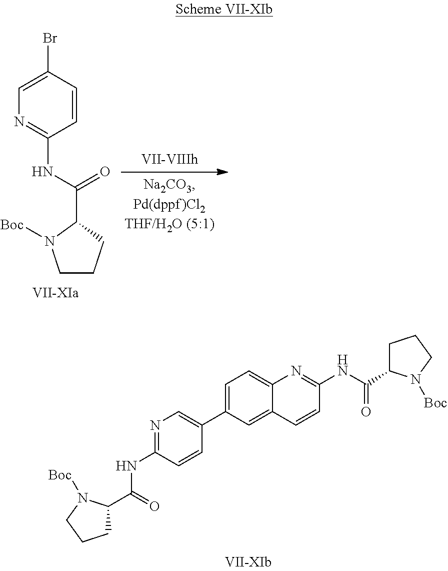

### General Procedure VII-AU

A mixture of compound VII-XIa (0.51 g, 1.22 mmol), compound VII-VIIIh (0.45 g, 1.22 mmol), Na₂CO₃ (0.26 g, 2.44 mmol) and Pd(dppf)Cl₂ (71 mg, 0.098 mmol) was dissolved in 20 mL THF and 4 mL H₂O. The reaction mixture was heated to reflux at 80° C. overnight. Then the mixture was concentrated under reduced pressure and the residue was combined with water, and then extracted with EtOAc. The combined organic phase extracts were dried over Na₂SO₄ and concentrated. The residue was purified by column chromatography to give compound VII-XIb (0.4 g, yield: 52%), MS (ESI) m/z (M+H)⁺ 631.3.

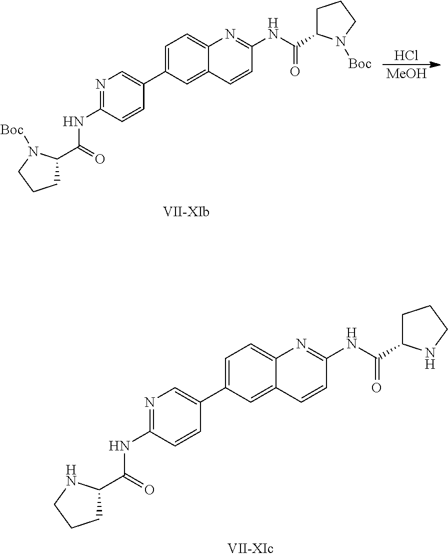

### General Procedure VII-AV

Compound VII-XIb (400 mg, 0.6 mmol) was dissolved in 100 mL HCl/MeOH. The mixture was stirred for 1.5 h at room temperature. Then concentrate the mixture under reduced pressure to give compound VII-XIc, which was used for next step without further purification.

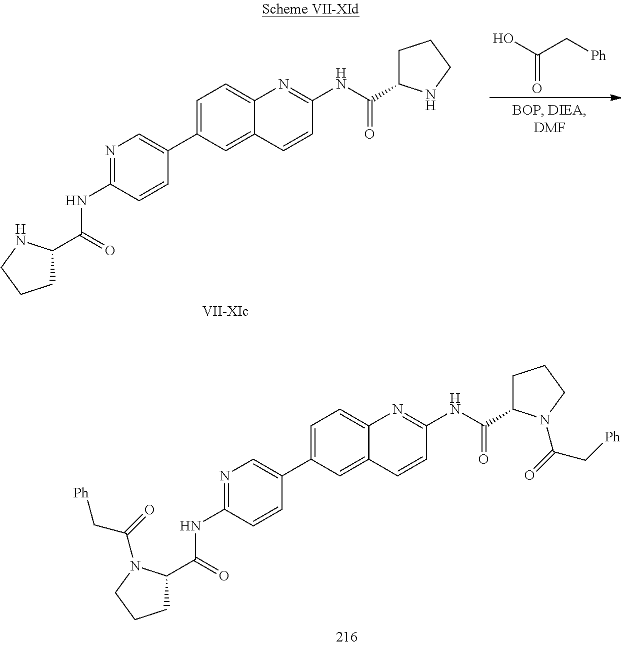

### General Procedure VII-AW

To a mixture of compound VII-XIc (200 mg, 0.465 mmol) and phenylacetic acid (152 mg, 1.12 mmol) in 8 mL DMF was added DIEA (480 mg, 3.7 mmol). The mixture was stirred for 30 min at room temperature. Subsequently BOP was added to the stirring mixture (617 mg, 1.4 mmol), and the mixture was stirred at room temperature overnight. After concentration, the residue was purified directly by prep-HPLC to afford compound 216 (60 mg, yield: 19.4%). ¹H NMR (300 MHz, DMSO-d₆) δ 10.90 (s, 1H), 10.67 (s, 1H), 8.76-8.75 (s, 1H), 8.36-8.33 (m, 1H), 8.27 (m, 3H), 8.24-8.23 (m, 1H), 8.19-8.15 (m, 1H), 8.05-8.02 (m, 1H), 7.85-7.82 (m, 10H), 4.59 (m, 2H), 3.67 (m, 4H), 3.60-3.55 (m, 4H), 2.12-2.11 (m, 2H), 1.90-1.83 (m, 6H), MS (ESI) m/z (M+H)⁺ 667.1.

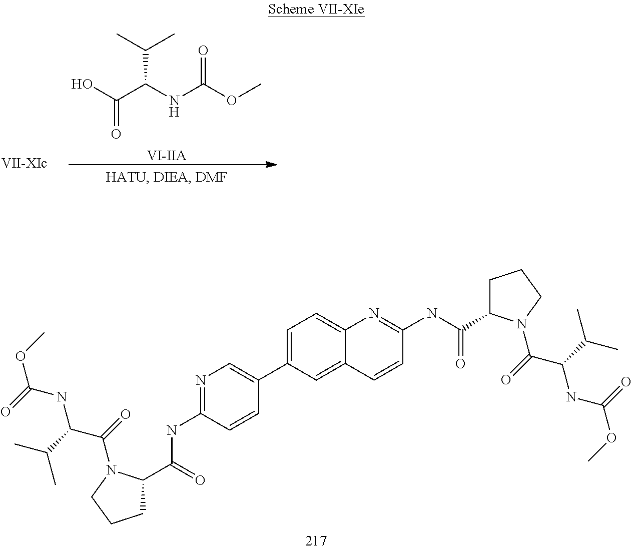

### General Procedure VII-AX

To a mixture of compound VI-IIA (195 mg, 1.12 mmol) and HATU (707 mg, 1.86 mmol) in 8 mL DMF was added DIEA (480 mg, 3.7 mmol). The mixture was stirred for 30 min at room temperature. Then compound VII-XIc (200 mg, 0.46 mmol) was added, and the reaction mixture was stirred overnight at room temperature. After concentration, the residue was purified directly by prep-HPLC to afford compound 217 (50 mg, yield: 14%). ¹H NMR (300 MHz, DMSO-d₆) δ 10.90 (s, 1H), 10.70 (s, 1H), 8.80-8.75 (s, 1H), 8.45-8.39 (m, 1H), 8.32-8.25 (m, 3H), 8.15-8.12 (m, 1H), 8.08-8.05 (m, 1H), 7.82-7.88 (m, 2H), 4.65-4.55 (m, 2H), 4.02-3.95 (m, 2H), 3.88-3.75 (m, 2H), 3.65-3.55 (m, 2H), 3.52-3.45 (s, 6H), 2.25-2.10 (m, 2H), 2.02-1.72 (m, 8H), 0.93-0.91 (d, 3H), 0.87-0.84 (d, 3H), MS (ESI) m/z (M+H)⁺ 745.3.

### Example VII-XII

### Preparation of Compound 218

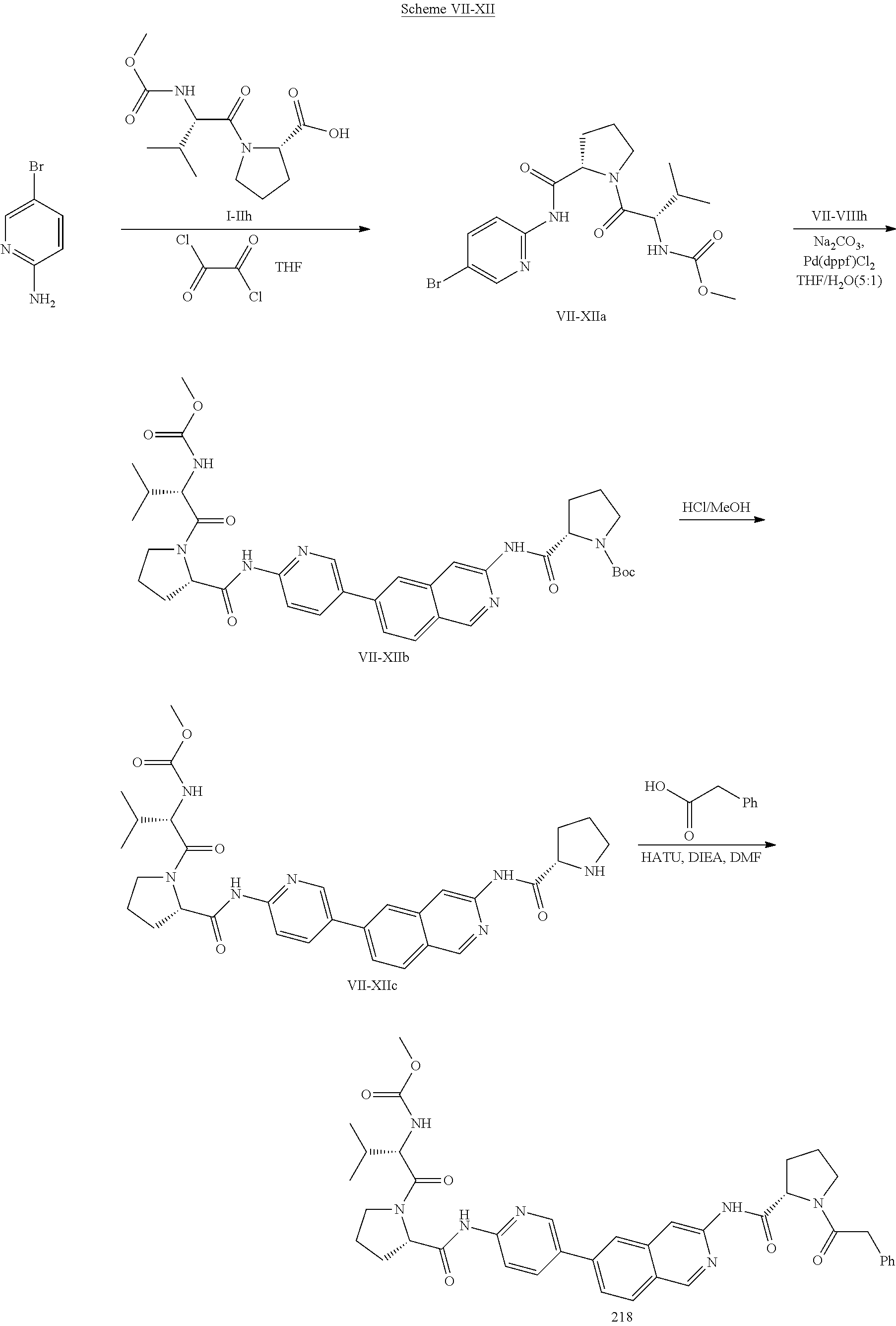

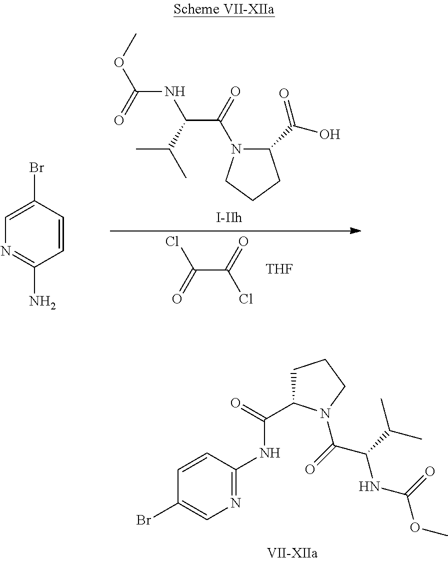

### General Procedure VII-AT

Compound I-IIh (2.59 g, 9.2 mmol) was dissolved in 150 mL THF under nitrogen cooled in ice water bath, added dry pyridine (3.7 mL, 45.8 mmol) followed by the dropwise addition of (COCl)₂ (2 mL, 22.9 mmol). A precipitate formed immediately. The reaction mixture was vigorous stirred at 0° C. for 2 h, then at ambient temperature for one hour. 100 mL THF was added, and filtered off solid. The filtrates was concentrate and the residue was dissolved in 100 mL DCM under nitrogen at 0° C., then pyridine (3 mL) 2-amino-5-bromopyridine (1.3 g, 7.6 mmol) was added. The reaction mixture was stirred at room temperature for 3 hours, and then concentrated. The residue was purified by column chromatography to afford compound VII-XIIa (500 mg, yield: 16%), MS (ESI) m/z (M+H)⁺ 428.9.

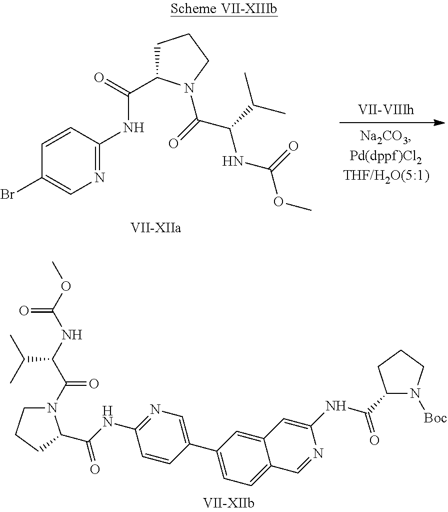

### General Procedure VII-AU

A mixture of compound VII-XIIa (0.36 g, 0.856 mmol), compound VII-VIIIh (0.4 g, 0.856 mmol), Na₂CO₃ (0.18 g, 1.7 mmol) and Pd(dppf)Cl₂ (62 mg, 0.085 mmol) was dissolved in 20 mL THF and 4 mL H₂O. The reaction mixture was heated to reflux at 80° C., and stirred overnight. Then the mixture was concentrated under reduced pressure and the residue was added water, extracted with EtOAc. Combined organic phase extracts was dried over Na₂SO₄. Then concentrated organic layer and the residue was purified by column chromatography to give compound VII-XIIb (0.4 g, yield 59%), MS (ESI) m/z (M+H)⁺ 688.3.

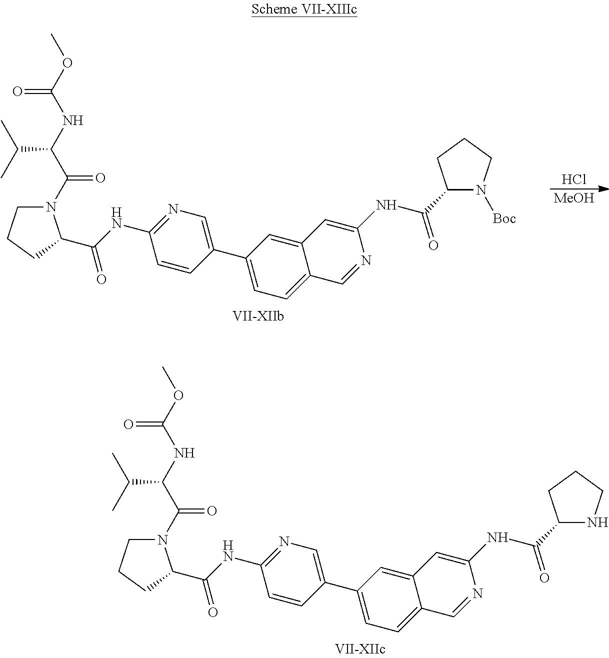

### General Procedure VII-AV

Compound VII-XIIb (400 mg, 0.875 mmol) was dissolved in 10 mL HCl/MeOH. The mixture was stirred for 1.5 h at room temperature. Then concentrate the mixture under reduced pressure to give compound VII-XIIc, which was used for next step without further purification.

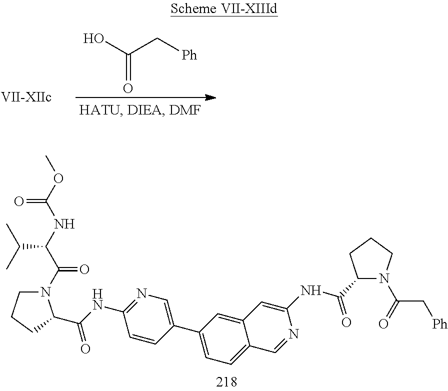

### General Procedure VII-AW

To a mixture of 2-phenylacetic acid (47.5 mg, 0.35 mmol) and HATU (228 mg, 0.6 mmol) in 8 mL DMF was added DIEA (155 mg, 1.2 mmol). The mixture was stirred for 30 min at room temperature. Compound VII-XIIc (200 mg, 0.29 mmol) was then was added to the stirring mixture, and the reaction mixture was stirred overnight at room temperature. Then 10 mL of water and 30 mL of EtOAc was added, the organic phase extracts was separated, dried over Na₂SO₄ and concentrated. The residue was purified by prep-HPLC to afford compound 218 (50 mg, yield: 24.4%). ¹H NMR (300 MHz, DMSO-d₆) δ10.90 (s, 1H), 10.71 (s, 1H), 8.77-8.76 (s, 1H), 8.37-8.34 (m, 1H), 8.28-8.25 (m, 2H), 8.24-8.21 (m, 1H), 8.18-8.11 (m, 1H), 8.05-8.02 (m, 1H), 7.86-7.83 (m, 10H), 7.36-7.33 (m, 1H), 7.30-7.16 (m, 5H), 4.62-4.60 (m, 2H), 4.02-3.96 (m, 1H), 3.80 (m, 1H), 3.68 (m, 2H), 3.61-3.58 (m, 3H), 3.52-3.49 (m, 3H), 2.15-2.13 (m, 2H), 2.03-1.87 (m, 7H), 0.93-0.91 (d, 3H), 0.87-0.83 (d, 3H), MS (ESI) m/z (M+H)⁺ 706.1.

### Example VII-XIII

### Preparation of Compound 219

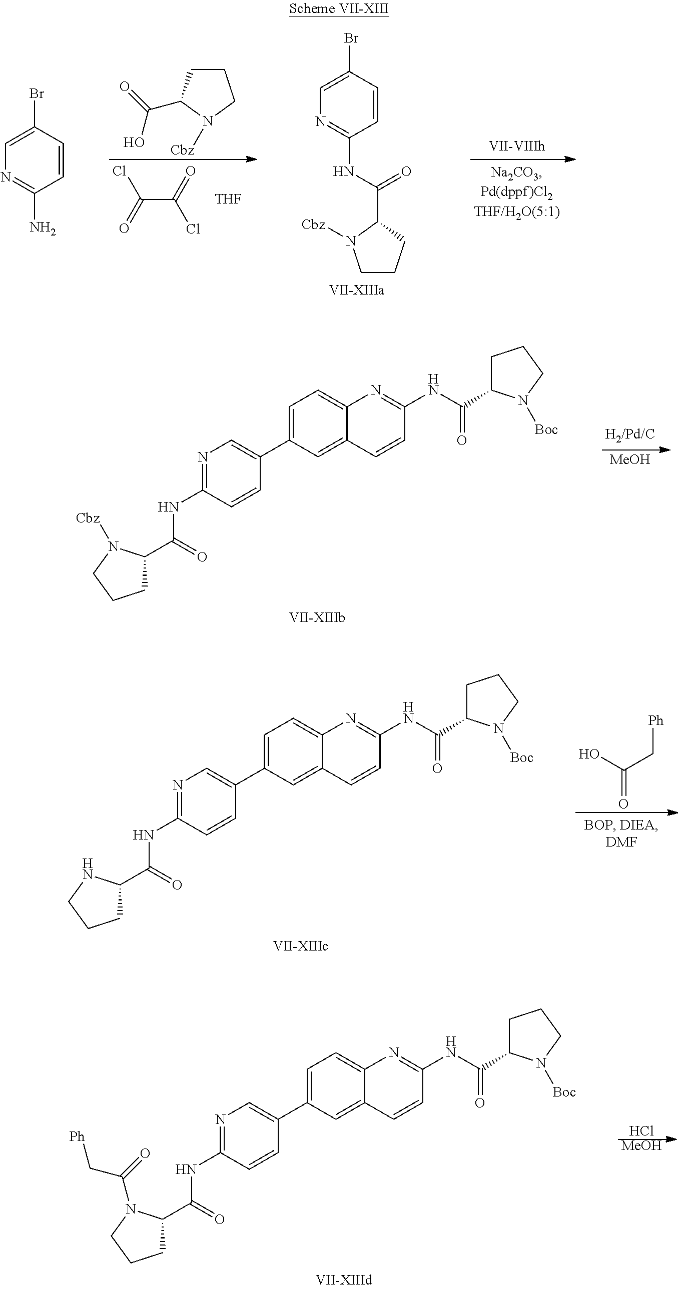

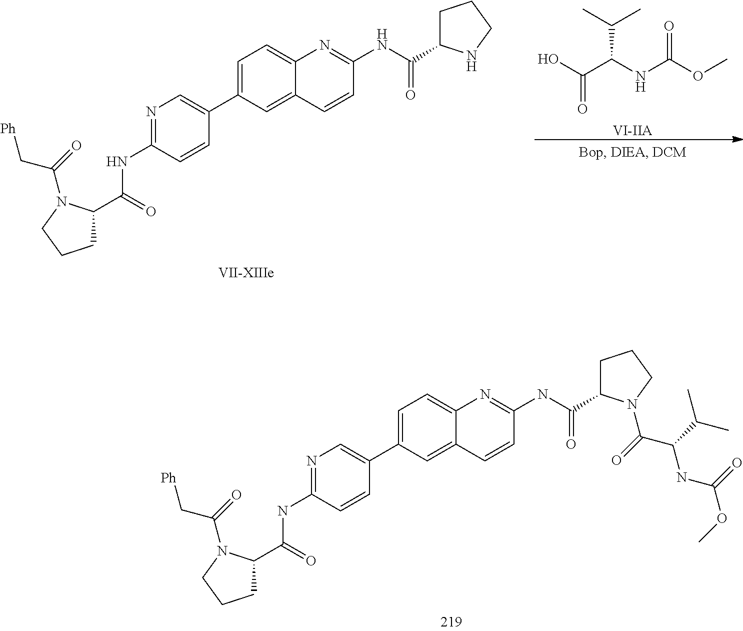

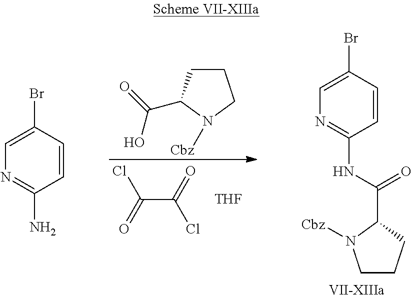

### General Procedure VII-AX

Pyridine (3.7 mL, 45.8 mmol) was added to a mixture of Cbz-N-proline (2.3 g, 9.2 mmol) in 150 mL THF under nitrogen at 0° C., then oxalyl dichloride (2 mL, 22.9 mmol) was added. A precipitate formed immediately. The reaction mixture was vigorously stirred at 0° C. for 2 h, then at ambient temperature for one hour. 100 mL of THF was added and filtered, the filtrates were concentrated, and the residue was dissolved in 100 mL DCM. 2-Amino-5-bromopyridine (1.32 g, 9.2 mmol) and 3 mL pyridine was added at 0° C. The reaction mixture was allowed to warm to room temperature, stirred for 3 hours, then concentrate. The residue was purified by column chromatography to afford compound VII-XIIIa (1 g, yield: 33%). MS (ESI) m/z (M+H)⁺ 403.7.

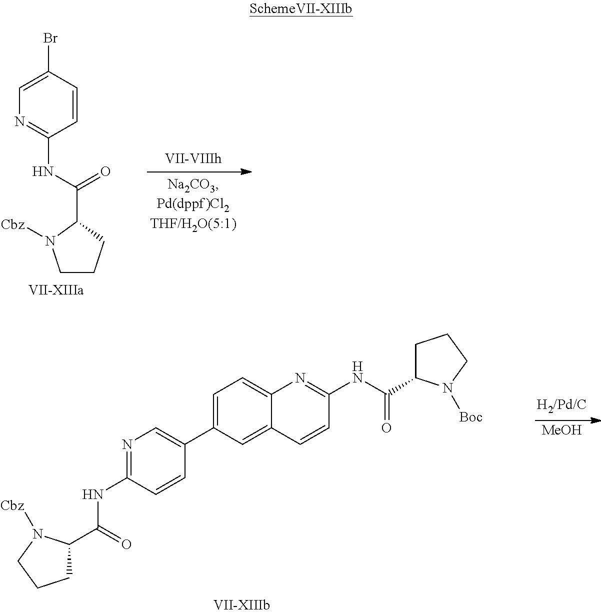

### General Procedure VII-AY

A mixture of compound VII-XIIIa (300 mg, 0.744 mmol), compound VII-VIIIh (347 mg, 0.744 mmol), Na₂CO₃ (158 mg, 1.49 mmol) and Pd(dppf)Cl₂ (54 mg, 0.074 mmol) was dissolved in 25 mL THF and 5 mL H₂O. The reaction mixture was heated to reflux at 80° C., and stirred overnight. After this the mixture was concentrated under reduced pressure and the residue was added water, and extracted with EtOAc. The organic phase extracts were combined and dried over Na₂SO₄. The organic layer was then concentrated and the residue was purified by column chromatography to afford compound VII-XIIIb (300 mg, yield: 60%). MS (ESI) m/z (M+H)⁺ 665.2.

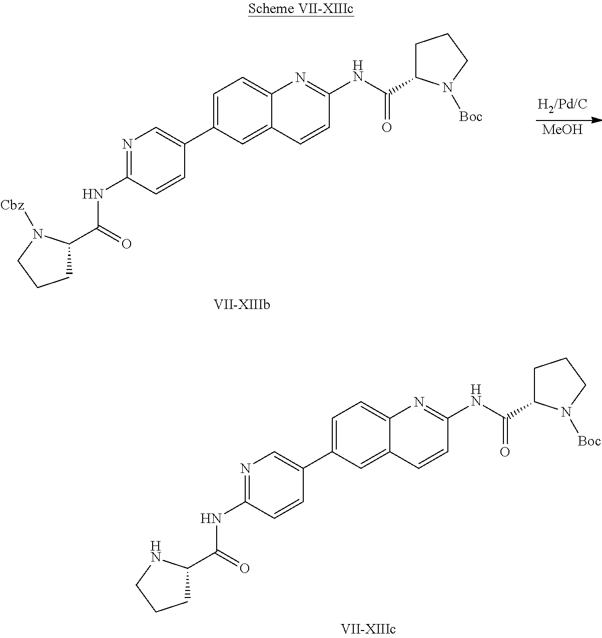

### General Procedure VII-AZ

A mixture of compound VII-XIIIb (140 mg, 0.2 mmol) and 10% Pd/C (100 mg) was dissolved in 20 mL MeOH. The reaction was stirred for 24 h under 30 psi of H₂ at ambient temperature. Then filtered the mixture to remove Pd/C, and concentrate the filtrates. The residue was purified by TLC preparation chromatography (DCM:MeOH=10:1) to give compound VII-XIIIc (40 mg, yield 35.7%). MS (ESI) m/z (M+H)⁺ 531.1.

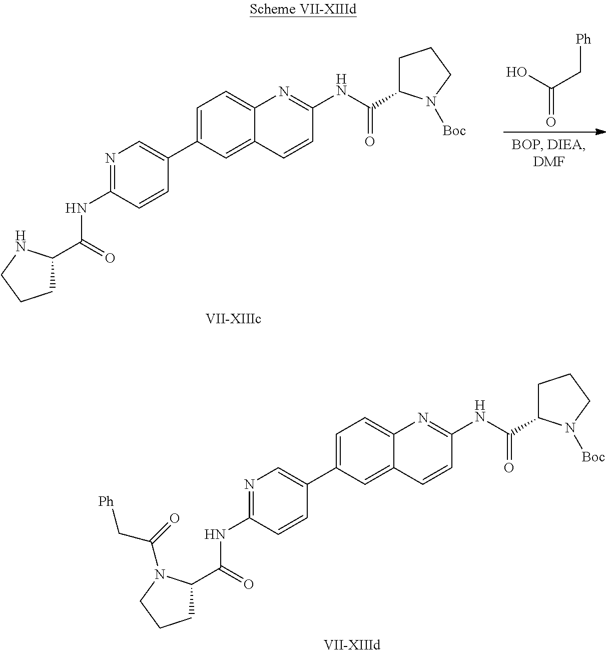

### General Procedure VII-BA

To a mixture of compound VII-XIIIc (40 mg, 0.075 mmol) and 2-phenylacetic acid (12 mg, 0.09 mmol) in 20 mL DCM was added DIEA (40 mg, 0.3 mmol). The mixture was stirred for 30 min at room temperature and then treated with BOP (40 mg, 0.09 mmol). The reaction mixture was stirred overnight at room temperature. The crude mixture was purified directly by Prep. TLC (PE:EA=1:1) to afford compound VII-XIIId (60 mg, yield: 85%). MS (ESI) m/z (M+H)⁺ 649.1.

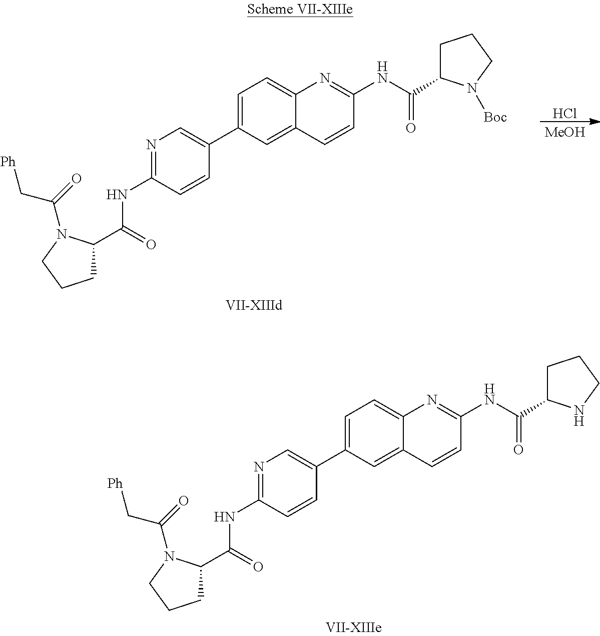

### General Procedure VII-BB

Compound VII-XIIId (60 mg, 0.093 mmol) was dissolved in 20 mL HCl/MeOH. The mixture was stirred for 2 h at room temperature. Then concentrate the mixture under reduced pressure to give compound VII-XIIIe, which was used for next step without further purification.

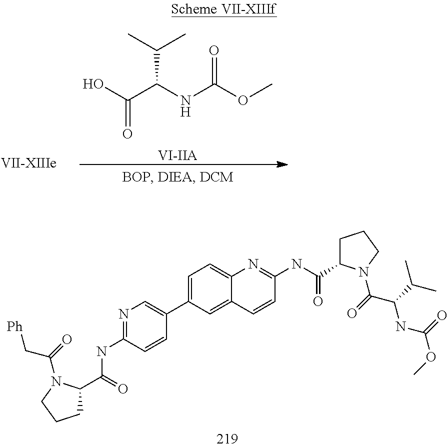

### General Procedure VII-BC

To a mixture of compound VII-XIIIe (100 mg, 0.18 mmol) and compound VI-IIA (32 mg, 0.18 mmol) in 20 mL DCM was added DIEA (90 mg, 0.73 mmol). The mixture was stirred for 30 min at room temperature and then BOP (97 mg, 0.11 mmol) was added, and the resulting mixture was stirred overnight at room temperature. The mixture was partitioned with water and extracted with DCM (50 mL×3). The combined organic layer extracts were dried over Na₂SO₄, concentrated and the resulting residue was purified by Prep. HPLC to afford compound 219 (27 mg, yield: 20%). ¹H NMR (400 MHz, CDCl₃) δ9.64 (s, 1H), 9.46 (s, 1H), 8.55 (s, 1H), 8.29-8.27 (d, 1H), 8.23-8.21 (d, 1H), 8.17-8.15 (d, 1H), 7.95-7.88 (d, 2H), 7.84 (d, 2H), 7.77-7.75 (d, 2H), 7.27 (m, 5H), 5.49-5.48 (d, 1H), 4.76-4.74 (d, 2H), 4.34-4.30 (t, 2H), 3.81-3.77 (d, 1H), 3.69 (s, 2H), 3.67-3.64 (m, 1H), 3.58 (s, 3H), 3.55-3.53 (m, 1H), 3.48-3.42 (q, 1H), 2.38-2.35 (m, 2H), 2.13-1.83 (m, 7H), 0.81-0.79 (d, 3H), 0.73 (d, 3H), MS (ESI) m/z (M+H)⁺ 706.2.

### Example VII-XIV

### Preparation of Compound 220

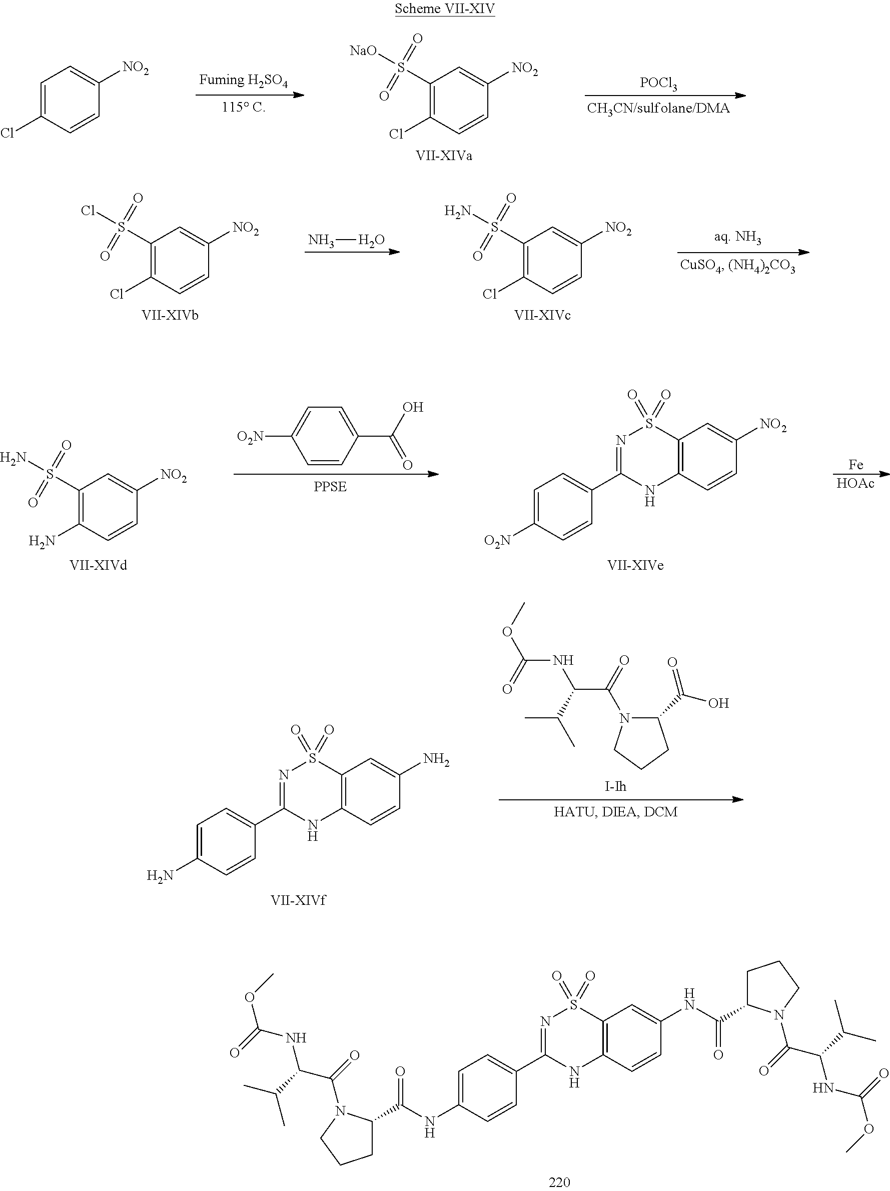

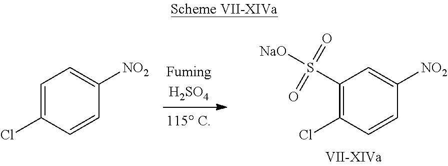

### General Procedure VII-BD

To molten 1-chloro-4-nitrobenzene (20 g, 127 mmol) was added 50% fuming sulfuric acid (22 g, 140 mmol) at 85° C., then the mixture was stirred at 115° C. for 16 hrs. After being cooled to r.t., the mixture was carefully poured into water while stirring, then 48% NaOH was added, and the precipitated solid was collected by filtration, washed with water and dried to afford compound VII-XIVa (25 g, yield 76%). ¹H NMR: (DMSO-d₆, 400 MHz) δ 8.63 (d, J=1.6 Hz, 1H), 8.19 (dd, J=2.4 Hz, 8.4 Hz, 1H), 7.73 (d, J=8.8 Hz, 1H).

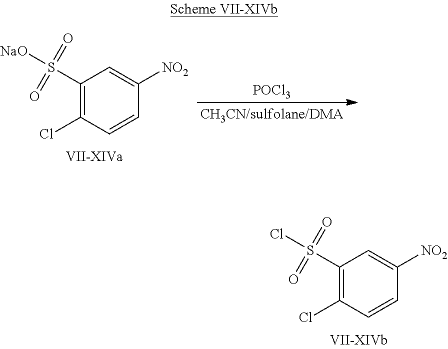

### General Procedure VII-BE

POCl₃ (8.9 g, 58.1 mmol) was added to a mixture of compound VII-XIVa (5 g, 19.4 mmol) in CH₃CN (5 mL), sulfolane (20 mL) and DMA (1 mL), the reaction mixture was stirred at reflux for 3 hrs. After being cooled to r.t., the mixture was poured into ice water, and extracted with EtOAc. The organic layer was separated, dried over Na₂SO₄ and concentrated to afford compound VII-XIVb (4 g, yield 81%).

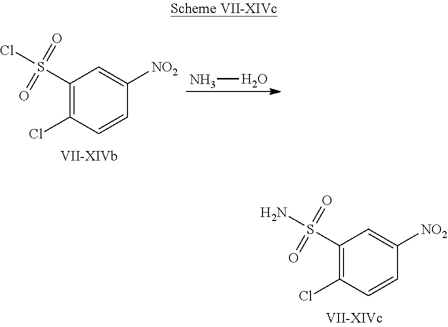

### General Procedure VII-BF

A mixture of compound VII-XIVb (4 g, 15.7 mmol) in 40 mL of aqueous ammonia was stirred at r.t. for 1 h. Then the mixture was poured into water, the precipitate solid was collected by filtration, and dried to afford compound VII-XIVc (3 g, yield 81%). MS (ESI) m/z (M+H)⁺ 237.

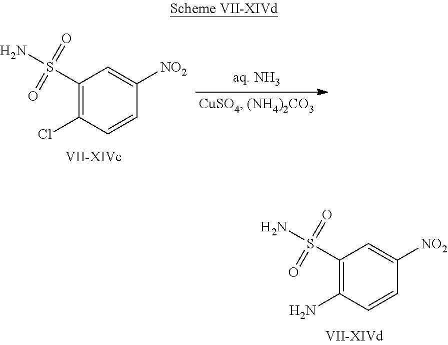

### General Procedure VII-BG

A mixture of compound VII-XIVc (3 g, 12.9 mmol), CuSO₄ (0.6 g, 3.76 mmol), (NH₄)₂CO₃ (3.0 g, 31 mmol) in 30 mL of aqueous ammonia was refluxed overnight. The mixture was cooled to r.t. and poured into water, the precipitated solid was collected by filtration, washed with water and dried to afford compound VII-XIVd (1.5 g, yield 54%). MS (ESI) m/z (M+H)⁺ 218.

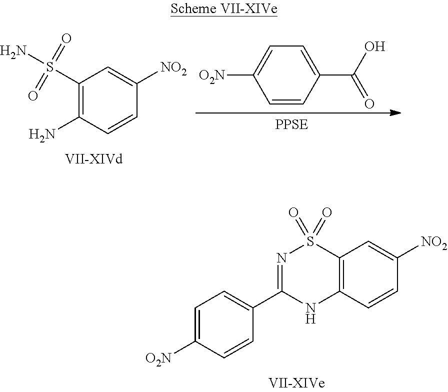

### General Procedure VII-BH

To a solution of polyphosphoric acid trimethylsilyl ester (PPSE, 5 mL) in toluene was added 4-nitrobenzoic acid (154 mg, 0.92 mmol), the mixture was stirred at 120° C. for 10 min. and then treated with compound VII-XIVd (200 mg, 0.92 mmol). The resulting mixture was refluxed overnight. After being cooled to r.t., the mixture was poured into water, the precipitated solid was collected and dried to afford compound VII-XIVe (100 mg, yield 31%). MS (ESI) m/z (M+H)⁺ 349.

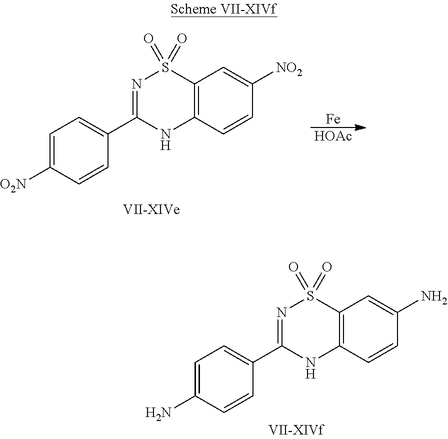

### General Procedure VII-BI

To a mixture of compound VII-XIVe (1 g, 2.88 mmol) in HOAc was added Fe powder (0.8 g, 14 mmol), the reaction mixture was stirred at 60° C. for 2 hrs. After filtration, the filtrate was concentrated under reduced pressure. The residue was purified by prep-HPLC to afford compound VII-XIVf (200 mg, yield 24%). MS (ESI) m/z (M+H)⁺ 289.

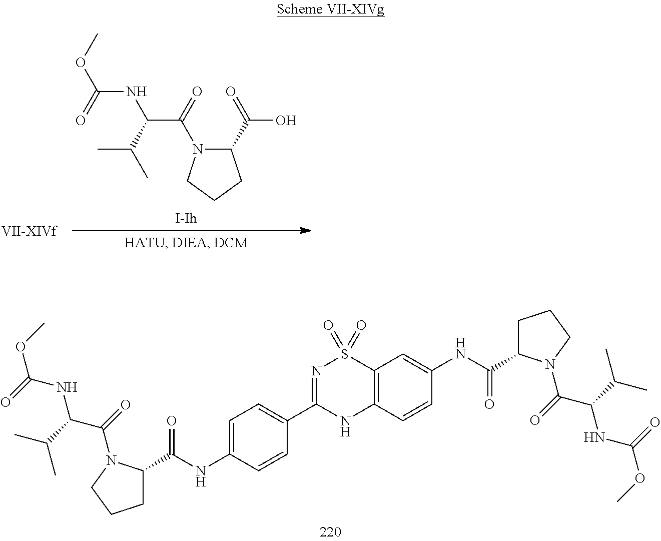

### General Procedure VII-BJ

A mixture of compound VII-XIVf (200 mg, 0.69 mmol), compound I-Ih (563 mg, 2.07 mmol), HATU (786 mg, 2.07 mmol) and DIEA (534 mg, 4.14 mmol) in DCM (6 mL) was stirred at 50° C. overnight. After completion of the reaction, the mixture was diluted with DCM (60 mL), washed with water and brine. The organic layer was separated, dried over Na₂SO₄ and concentrated in vacuo. The residue was purified by prep-HPLC to afford compound 220 (60 mg, yield 11%). ¹H NMR (CD₃OD, 400 MHz) δ 8.43 (s, 1H), 7.76 (d, J=8.8 Hz, 2H), 7.54 (d, J=8.8 Hz, 2H), 7.13-7.24 (m, 2H), 4.62-4.67 (m, 2H), 4.28 (d, J=6.4 Hz, 2H), 4.05 (br, 2H), 3.78-3.84 (m, 2H), 3.69 (s, 3H), 3.68 (s, 3H), 2.32-2.34 (m, 2H), 2.17-2.20 (m, 4H), 1.98-2.11 (m, 4H), 1.16 (d, J=6.4 Hz, 6H), 1.08 (d, J=6.8 Hz, 6H). MS (ESI) m/z (M+H)⁺ 797.5.

### Example VII-XV

### Preparation of Compound 221

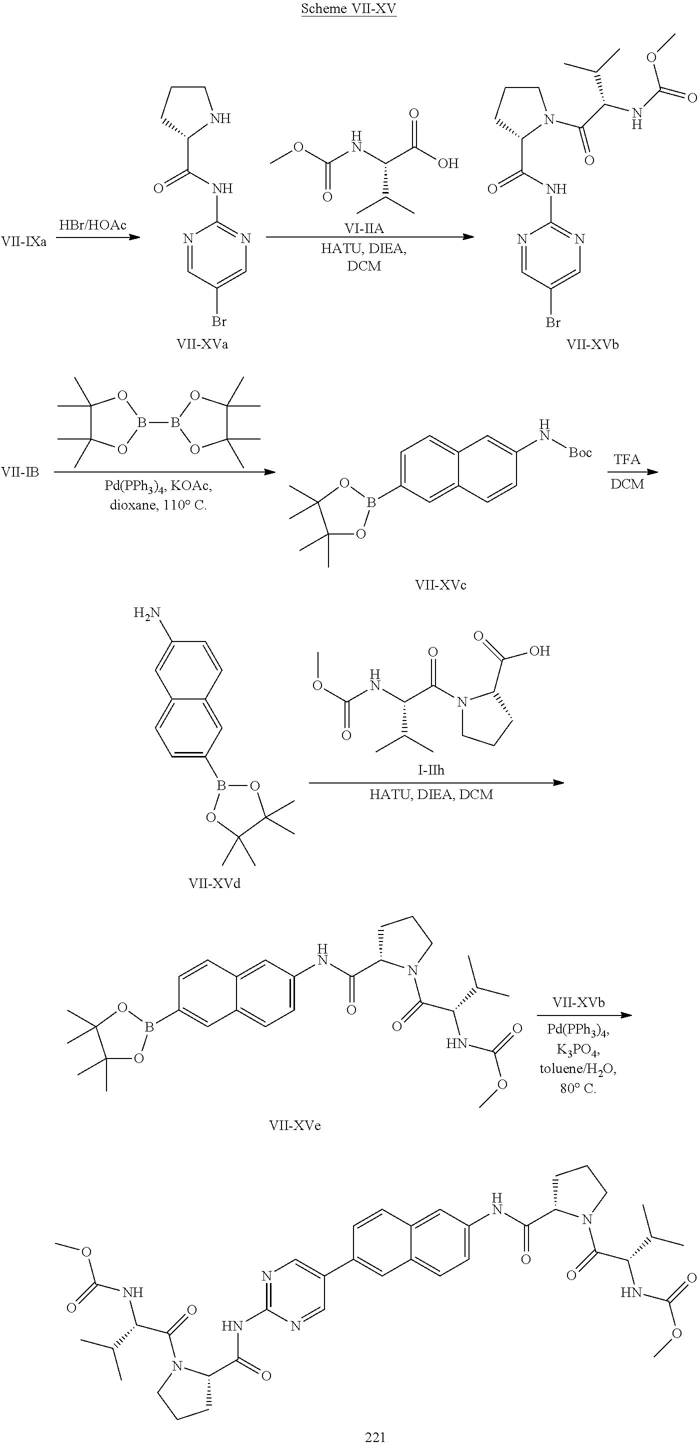

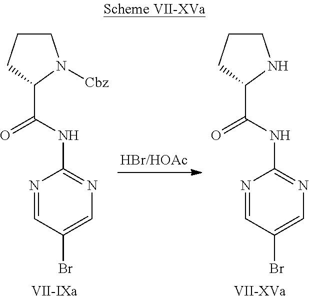

### General Procedure VII-BK

A mixture of compound VII-IXa (870 mg, 2.15 mmol) and 5 mL of HBr/HOAc solution (48%) was stirred for 2 hrs at room temperature. Then it was poured into ice-water (100 mL) and adjusted to pH=8 by progressively adding solid Na₂CO₃. The mixture was then extracted with DCM (100 mL×3). The combined organic layer extracts were washed with brine, dried over Na₂SO₄, concentrated and the resulting residue was purified by prep-TLC to afford compound VII-XVa (508 mg, yield 87%).

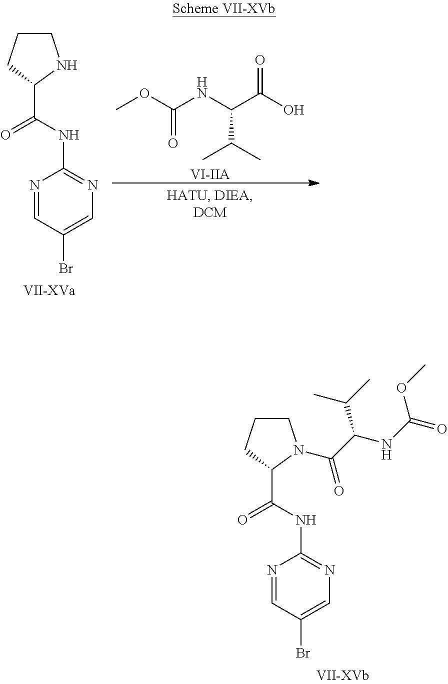

### General Procedure VII-BL

To a mixture of compound VII-XVa (508 mg, 1.85 mmol) and HATU (1.05 g, 2.78 mmol) in 10 mL of DCM was added DIEA (954 mg, 7.4 mmol). The mixture was stirred for 30 min at room temperature. Subsequently, compound VI-IIA (324 mg, 1.85 mmol) was added to the stirring mixture, and the mixture was stirred overnight at room temperature. After that, 30 mL water was added, and the mixture was extracted with EtOAc (30 mL×3). The combined organic phase extracts were washed with brine and dried over Na₂SO₄, concentrated and the resulting residue was purified by prep-HPLC to provide compound VII-XVb (300 mg, yield 38%).

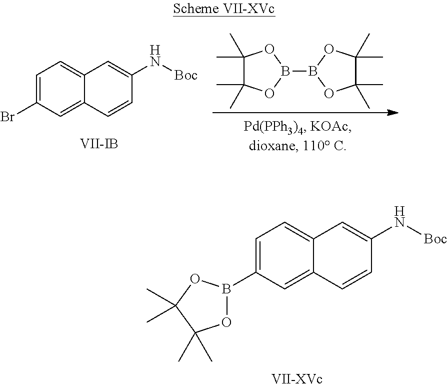

### General Procedure VII-BM

A mixture of compound VII-IB (1.5 g, 4.69 mmol), Bis(pinacolato)diborane (1.7 g, 7 mmol), Pd(PPh₃)₄ (265 mg, 0.234 mmol) and KOAc (3.9 g, 40.7 mmol) was dissolved in 30 mL of dioxane, the mixture was purged with nitrogen. Then the mixture was heated to reflux at 110° C. for 8 hrs under a nitrogen atmosphere. After the completion of reaction, the reaction mixture was cooled to r.t. and concentrated, the resulting residue was purified by column chromatography to afford compound VII-XVc (1 g, yield 58%).

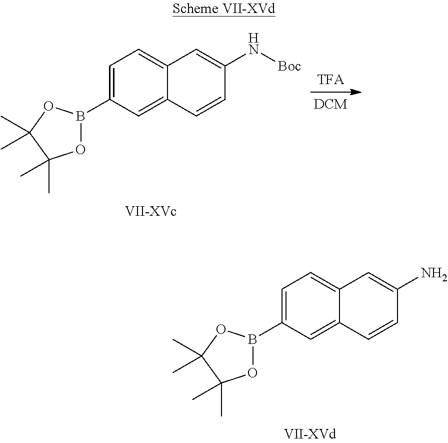

### General Procedure VII-BN

Compound VII-XVc (1 g, 2.7 mmol) was dissolved in 10 mL of DCM and TFA (2 mL). The mixture was stirred for 2 hrs at room temperature. Then the mixture was concentrated under reduced pressure, 30 mL of water was added to the residue, and residual acid was neutralized with aq. NaHCO₃, followed by extraction with EtOAc (70 mL×3). The combined extracts were washed with brine, dried over Na₂SO₄, and concentrated to afford compound VII-XVd (773 mg, yield 100%).

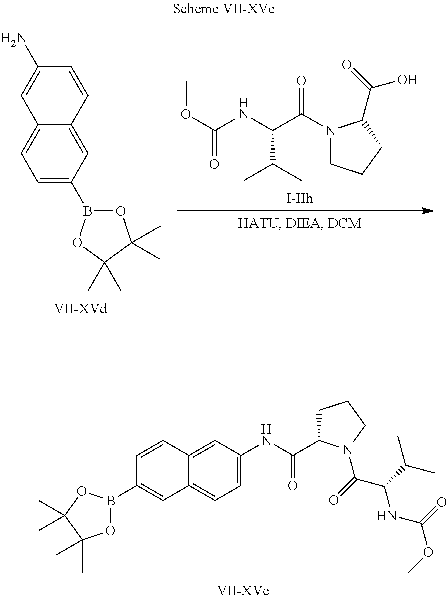

### General Procedure VII-BO

To a mixture of compound VII-XVd (560 mg, 2.08 mmol) and HATU (1.5 g, 4 mmol) in 10 mL of DCM was added DIEA (1.0 g, 8 mmol). The mixture was stirred for 30 min at room temperature and then compound I-Ih (560 mg, 2.08 mmol) was added, the resulting mixture was stirred overnight at room temperature. Subsequently, 30 mL of water was added and the mixture was extracted with EtOAc (70 mL×3). The combined extracts were washed with brine, dried over Na₂SO₄, and concentrated, the residue was purified by Prep-TLC to afford compound VII-XVe (600 mg, yield 55%).

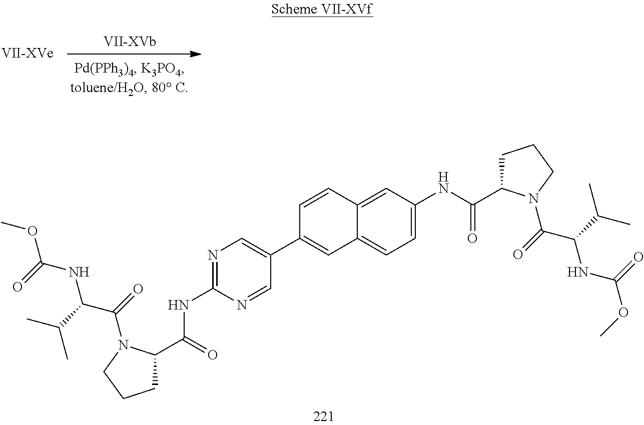

### General Procedure VII-BP

To a solution of compound VII-XVe (60 mg, 0.115 mmol) in toluene/H₂O (3 mL) was added compound VII-XVb (50 mg, 0.115 mmol), K₃PO₄ (49 mg, 0.23 mmol) and Pd(PPh₃)₄ (8 mg, 0.0115 mmol). The mixture was purged with N₂ and heated at 80° C. overnight under nitrogen protection. LCMS indicated disappearance of starting material. The mixture was diluted with water (100 mL) and extracted with EtOAc (50 mL×3). The combined organic layers were washed by brine, dried over Na₂SO₄, and concentrated and the resulting residue was purified by Prep-HPLC to afford compound 221 (10 mg, yield 11%). MS (ESI) m/z (M+H)⁺ 745.4.

### Example VII-XVI

### Preparation of Compound 222

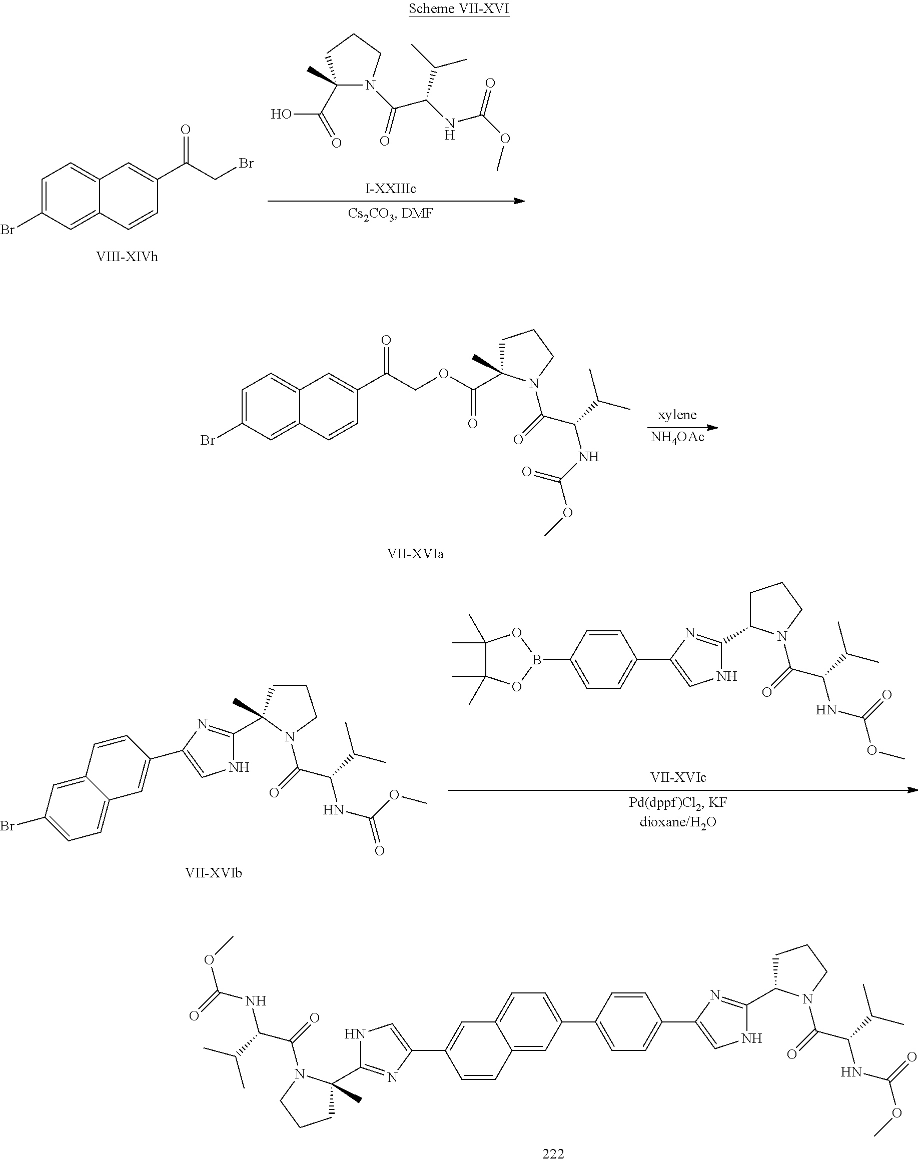

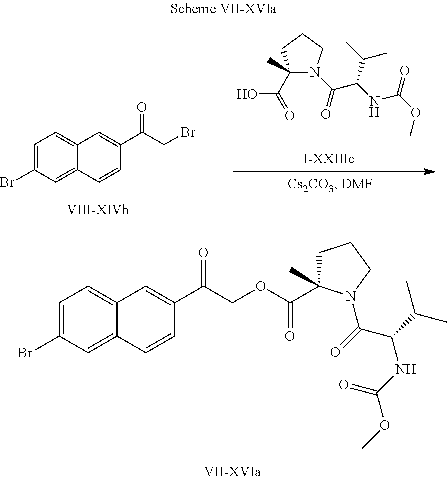

### General Procedure VII-BP

A mixture of compound VIII-XIVh (300 mg, 0.914 mmol), compound I-XXIIIc (300 mg, 1.37 mmol) and Cs₂CO₃ (892 mg, 7.74 mmol) in DMF (5 mL) was stirred at room temperature for 2 hours. The mixture was then diluted with EtOAc (30 mL), and washed with brine. The organic layer was separated, dried over anhydrous Na₂SO₄ and concentrated to give crude compound VII-XVIa (400 mg, yield 82%). MS (ESI) m/z (M+H)⁺ 533.

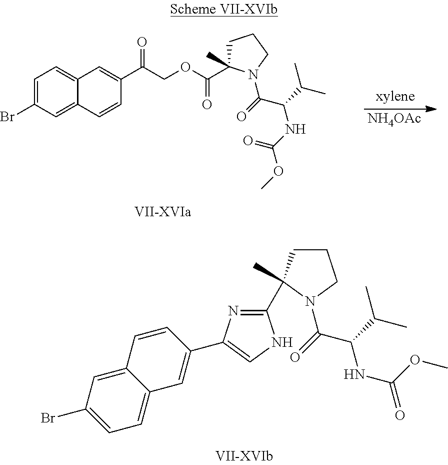

### General Procedure VII-BP

A mixture of compound VII-XVIa (400 mg, 0.75 mmol) and NH₄OAc (867 mg, 11.2 mmol) in 4 mL of xylene was stirred at 140° C. overnight in a sealed tube. After being cooled to r.t., the solvent was removed under reduce pressure and the residue was diluted with EtOAc (20 mL), and washed with brine. The organic layer was separated, dried over anhydrous Na₂SO₄ and concentrated. The residue was purified by prep-TLC (PE/EA=1/1) to afford of compound VII-XVIb (200 mg, yield 52%). MS (ESI) m/z (M+H)⁺ 513.

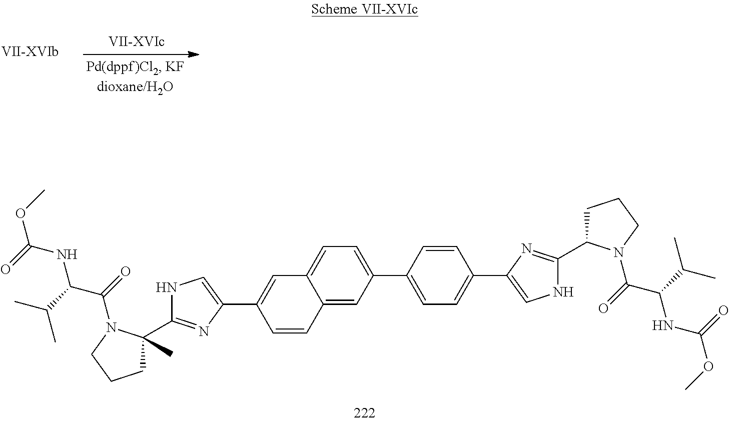

### General Procedure VII-BQ

To a mixture of compound VII-XVIb (100 mg, 0.19 mmol), compound VII-XVIc (174 mg, 0.35 mmol) and KF (73 mg, 0.78 mmol) in 1,4-dioxane (3 mL) and H₂O (0.4 mL) was added Pd(dppf)Cl₂ (5 mg) under N₂ protection, and the mixture was stirred at 95° C. for 4 hrs. After dilution with EtOAc (30 mL), the organic layer was washed with brine, dried over Na₂SO₄, and concentrated under reduced pressure. The residue was purified by Prep-HPLC to afford compound 222 (25 mg, yield 16%). ¹H NMR (400 MHz, CD₃OD): δ 8.10-8.19 (m, 3H), 7.79-7.94 (m, 9H), 7.41 (s, 1H), 7.37 (s, 1H), 5.19-5.22 (m, 1H), 4.22-4.27 (m, 2H), 3.89-4.10 (m, 4H), 3.67 (s, 6H), 2.55-2.60 (m, 2H), 2.07-2.39 (m, 8H), 1.91 (s, 3H), 1.02-0.81 (m, 12H). MS (ESI) m/z (M+H)⁺ 803.6.

### Section VIII

### Preparation of Compounds

### Section VIII

### Example VIII-I

### Preparation of Compound 401

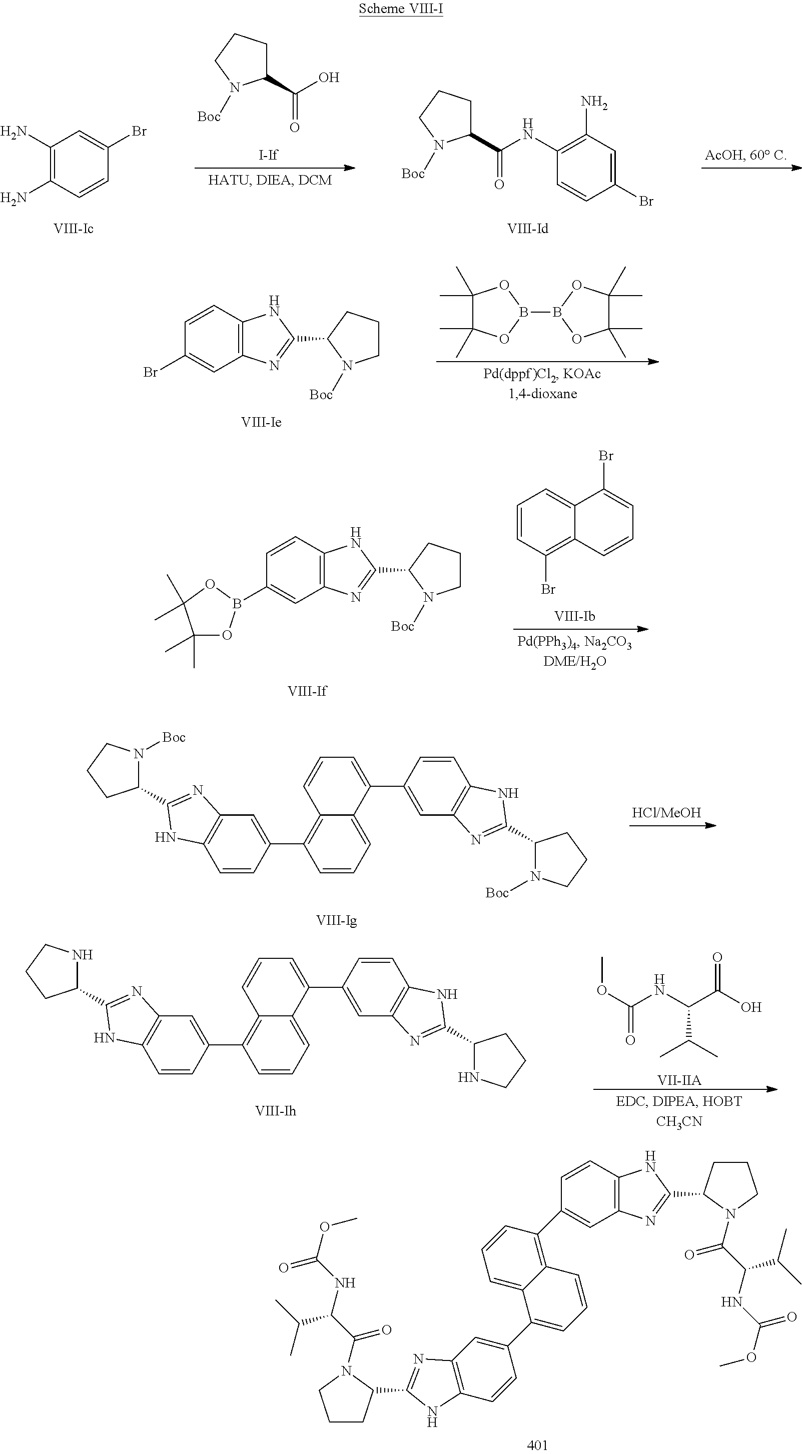

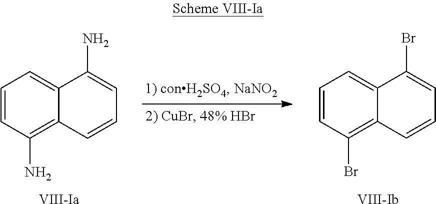

### General Procedure VIII-A

To a solution of con. H₂SO₄ (14 mL) in water (100 mL) was added naphthalene-1,5-diamine (VIII-Ia) (8 g, 50.6 mmol). To the resulting solution, a solution of NaNO₂ (7.8 g, 116.3 mmol) in water (50 mL) was added dropwise. The resulting mixture was stirred at 0° C. for 45 minutes. Then CuBr (20 g, 25.3 mmol), HBr in AcOH (48%, 180 mL) and water (200 mL) was added. The solution was stirred at the same temperature for 1 hour, 2 hours at r.t. and then heated at 70° C. for 30 minutes. The organic layer was separated with toluene and concentrated under reduced pressure. The residue was purified by column chromatograph on silica gel (eluting with petroleum ether) to afford 1,5-dibromonaphthalene (VIII-Ib) (5.2 g, yield 40%) as a pale yellow solid. ¹H NMR (300 MHz, CDCl₃) δ 8.26 (d, J=12.0 Hz, 2H), 7.84 (d, J=10.0 Hz, 2H), 7.43 (m, 2H).

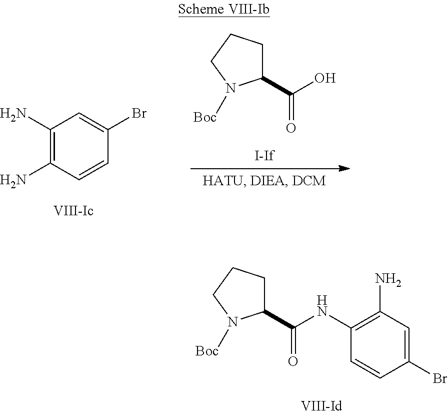

### General Procedure VIII-B

To a solution of 4-bromobenzene-1,2-diamine (VIII-Ic) (8.69 g, 46.46 mmol) in dry DCM (500 mL) was added HATU (35.3 g, 92.92 mmol), DIEA (10.69 g, 92.92 mmol). A solution of compound I-If in dry DCM (100 mL) was added into the above mixture dropwise. After addition, the reaction mixture was stirred at r.t. overnight until all the starting material was completely consumed on TLC (PE:EtOAc=1:1). The mixture, was diluted with water (300 mL) and extracted with DCM (300 mL×3). The combined organic layers were washed with brine, dried over sodium sulfate, and concentrated in vacuo. The crude product VIII-Id was used directly in the next step without further purification.

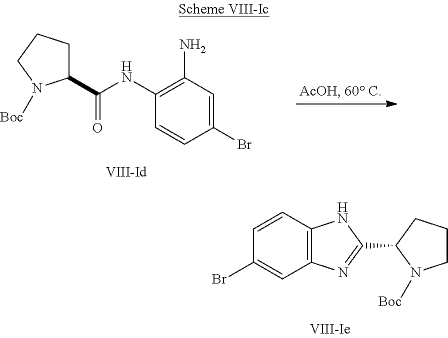

### General Procedure VIII-C

A solution of compound VIII-Id (12 g, 31.4 mmol) in AcOH (80 mL) was stirred at 60° C. for 4 hours at which time TLC (PE:EtOAc=1:2) indicated the starting material was consumed. The mixture was diluted with EtOAc (200 mL) and washed with saturated aq. NaHCO₃ (200 mL×5). The organic layer was concentrated and the residue was purified by column chromatograph on silica gel (eluting with PE:EtOAc=10:1 to 1:1) to give compound VIII-Ie (10 g, yield 59% over two steps) as a brown oil.

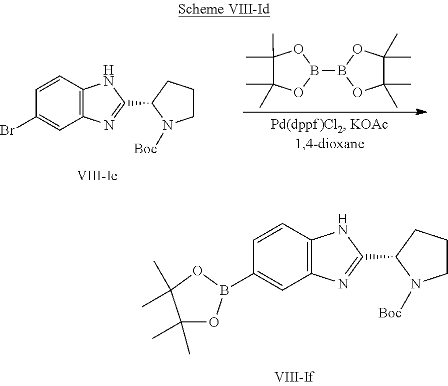

### General Procedure VIII-D

To a solution of compound VIII-Ie (5.2 g, 14.19 mmol) in 1,4-dioxane (100 mL) was added Bis(pinacolato)diboron (7.2 g, 28.38 mmol), KOAc (2.78 g, 28.38 mmol) and Pd(dppf)Cl₂ (0.5 g, catalyzed amount). The mixture was purged with N₂ for 5 minutes and heated to 80° C. overnight. LCMS showed the reaction was completed. The mixture was diluted with water (100 mL) and extracted with EtOAc (150 mL×3). The combined organic layers were washed with brine, dried over sodium sulfate, concentrated under vacuum. The residue was purified by column chromatograph on silica gel (eluting with PE:EtOAc=10:1 to 1:1) to afford compound VIII-If (4 g, yield 67%) as a brown oil. MS (ESI) m/z (M+H)⁺ 414.1

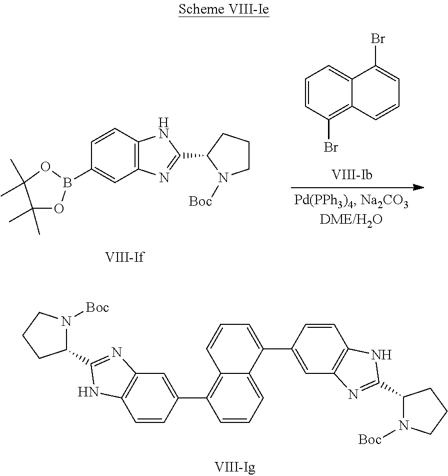

### General Procedure VIII-E

To a solution of compound VIII-If (0.89 g, 2.1 mmol) in DME/water (10 mL/1 mL) was added 1,5-dibromonaphthalene (VIII-Ib) (0.3 g, 1.05 mmol), Na₂CO₃ and Pd(PPh₃)₄ (0.05 g, catalyzed amount). The mixture was purged with N₂ and heated to 80° C. overnight under N₂. LCMS showed that the reaction was completed. The mixture was diluted with water (100 mL) and extracted with EtOAc (150 mL×3). The combined organic layers were washed with brine, dried over sodium sulfate, concentrated under vacuum. The residue VIII-Ig was used directly in the next step without further purification. MS (ESI) m/z (M+H)⁺ 699.1.

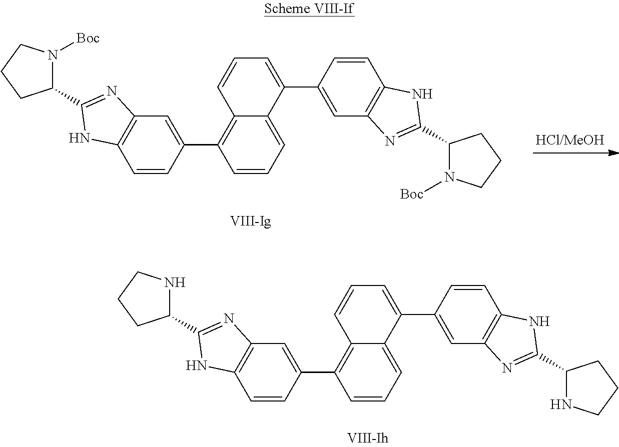

### General Procedure VIII-F

A solution of compound VIII-Ig (0.5 g, 0.72 mmol) in methanol (10 mL) was added hydrochloric acid in methanol (4 M, 5 mL) and the mixture was stirred at r.t. overnight. LCMS showed the reaction was complete. The reaction solution was concentrated under reduced pressure. The crude product VIII-Ih was used directly in the next step without further purification. MS (ESI) m/z (M+H)⁺ 499.1.

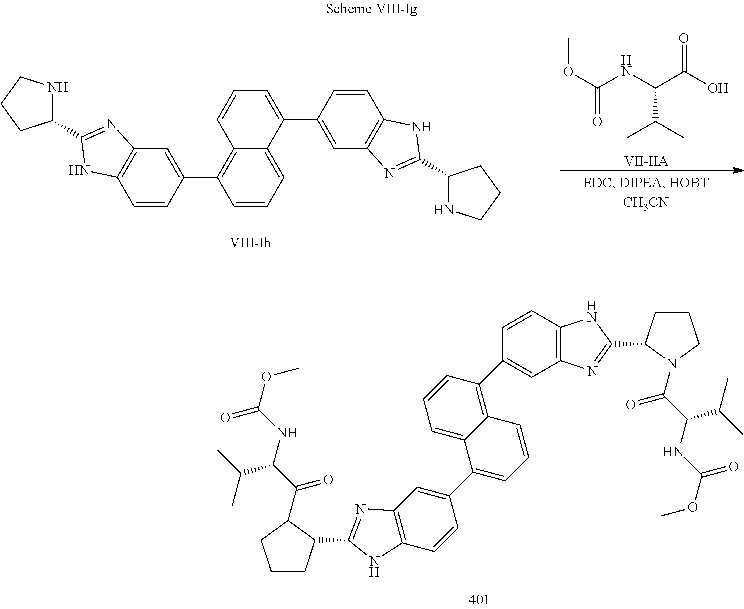

### General Procedure VIII-G

To s solution of compound VIII-Ih (0.4 g, 0.8 mmol) in anhydrous DCM (20 mL) was added compound VII-IIA (0.28 g, 1.6 mmol), EDC.HCl (0.37 g, 1.92 mmol) and HOBT (0.26 g, 1.92 mmol). The mixture was cooled to 0° C. and DIPEA (0.25 g, 1.92 mmol) was added dropwise. After addition the reaction mixture was stirred at r.t. for 4 hours. The mixture was diluted with water (20 mL) and extracted with EtOAc (50 mL×3). The combined organic layers were washed with brine, dried over sodium sulfate, and concentrated in vacuo. The residue was purified by Prep-HPLC to afford 401 (0.12 g, yield 15% over above three step) as a white solid. ¹H NMR (400 MHz, CDCl₃) δ 10.50 (d, J=11.2 Hz, 2H), 7.89 (m, 4H), 7.46 (m, 8H), 5.46 (m, 4H), 4.22 (d, J=9.6 Hz, 2H), 3.71 (m, 6H), 3.18 (m, 2 H), 2.25 (m, 10H), 1.07 (m, 12H). MS (ESI) m/z (M+H)⁺ 813.1.

### Example VIII-II

### Preparation of Compound 402

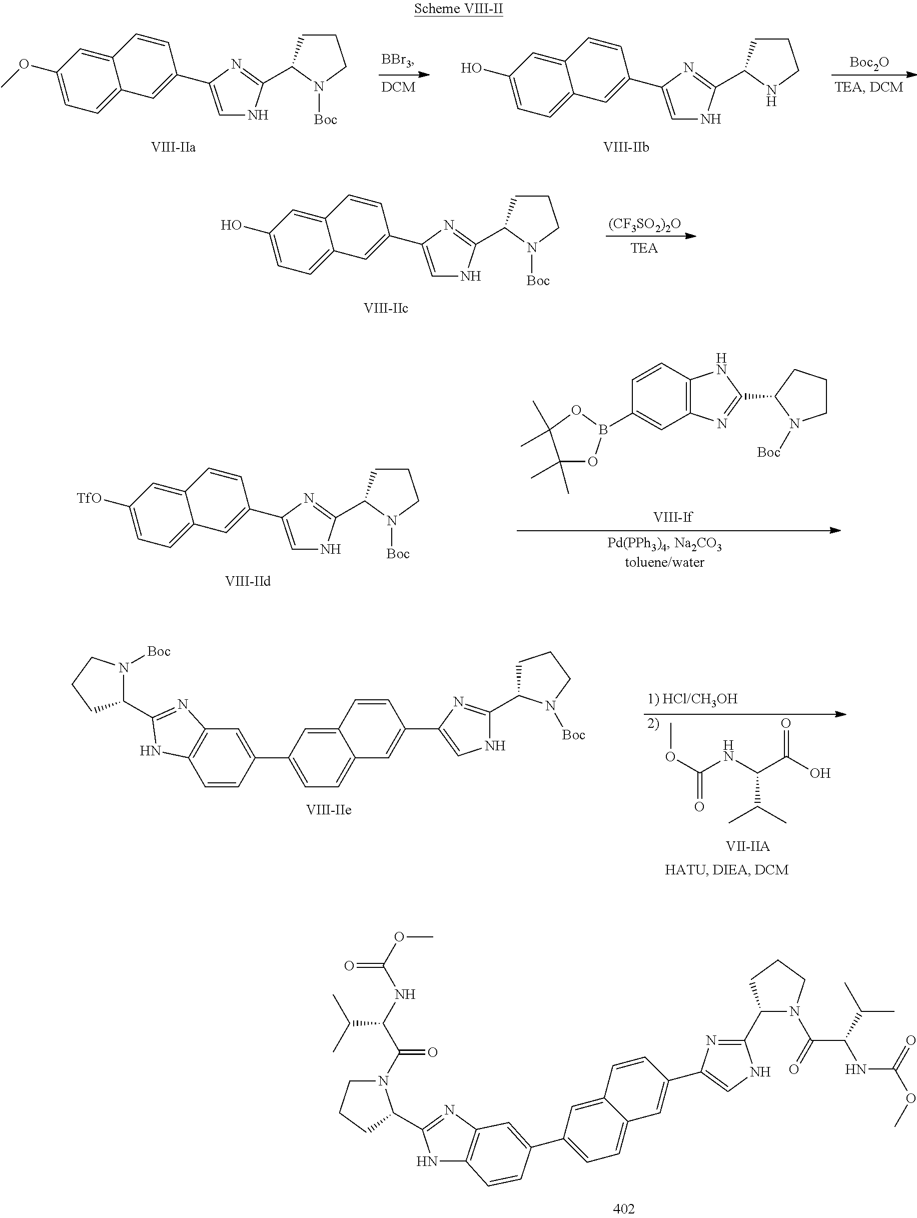

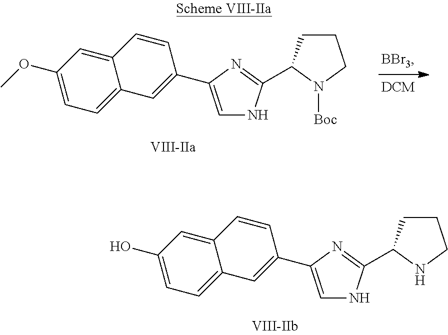

### General Procedure VIII-H

To a solution of compound VIII-IIa (1.1 g, 2.8 mmol) in DCM (40 mL) was added BBr₃ (0.85 mL, 8.4 mmol) at −60° C. to −70° C. dropwise. After addition, the mixture was stirred at r.t. for 2 hours. The reaction was quenched by adding methanol dropwise at −70° C. Subsequently, the mixture was poured into ice-water, and extracted with EtOAc (50 mL×3). The combined organic layers were washed with brine, dried over sodium sulfate, and concentrated in vacuo. The crude product VIII-IIb was used directly in the next step without further purification. MS (ESI) m/z (M+H)⁺ 280.

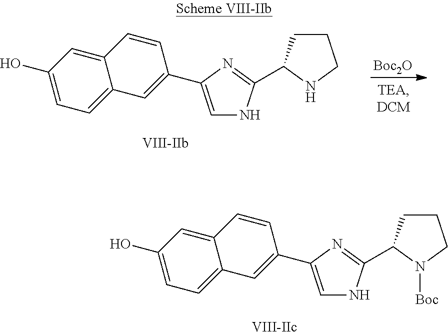

### General Procedure VIII-I

To a solution of compound VIII-IIb (0.7 g, 2.5 mmol) in DCM (40 mL) was added TEA (0.76 g, 7.5 mmol) and Boc₂O (0.66 g, 3.0 mmol). The mixture was stirred at r.t. overnight. The reaction mixture was concentrated under reduced pressure. The crude product was purified by column chromatograph on silica gel (eluting with PE/EA=3:1) to afford compound VIII-IIc (0.75 g, yield 79%) as a pale yellow solid. MS (ESI) m/z (M+H)⁺ 380.

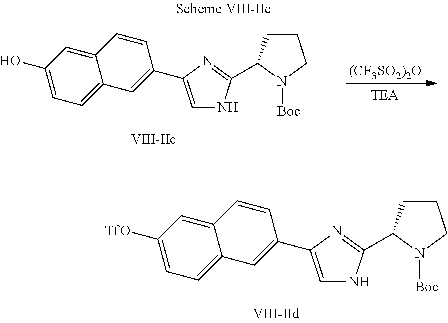

### General Procedure VIII-J

To a solution of compound VIII-IIc (0.75 g, 1.98 mmol) in anhydrous DCM (50 mL) was added (CF₃SO₂)₂O (0.4 mL, 2.37 mmol) at 0° C. dropwise. The mixture was stirred at r.t. for 3 hours. The reaction mixture was diluted with water (50 mL) and extracted with EtOAc (50 mL×3). The combined organic layers were washed with brine, dried over sodium sulfate, and concentrated in vacuo. The crude product was purified by column chromatograph on silica gel (eluting PE/EtOAc=3:1) to afford compound VIII-IId (0.14 g, yield 26%) as a white solid. MS (ESI) m/z (M+H)⁺ 512.

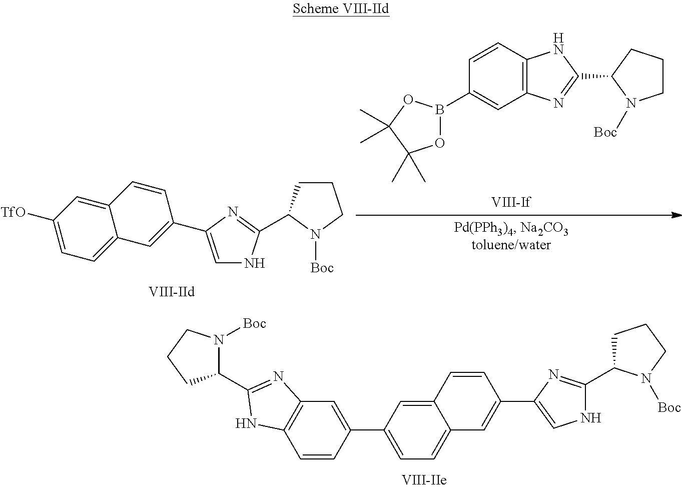

### General Procedure VIII-K

To a solution of compound VIII-IId (0.14 g, 0.27 mmol) in toluene/water (10 mill mL) was added compound VIII-If (0.13 g, 0.3 mmol), Na₂CO₃ (0.87 g, 0.8 mmol) and Pd(PPh₃)₄ (0.035 g, catalyzed amount). The mixture was purged with N₂ and heated to 80° C. overnight under N₂ protection. LCMS indicated that the reaction was completed. The mixture was diluted with water (30 mL) and extracted with EtOAc (100 mL×3). The combined organic layers were washed with brine, dried over sodium sulfate, and concentrated under reduced pressure. The residue was purified by column chromatography on silica gel (eluting PE/EtOAc=3:1) to afford compound VIII-IIe (0.1 g, yield 56%) as a white solid. MS (ESI) m/z (M+H)⁺ 649.

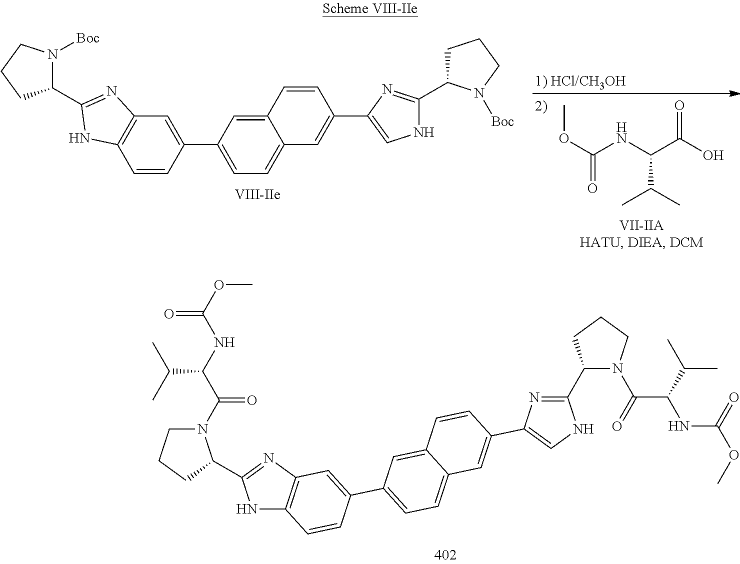

### General Procedure VIII-L

A solution of compound VIII-IIe (0.1 g, 0.15 mmol) in methanol (10 mL) was added a solution of hydrochloric acid in methanol (4 M, 5 mL) and the mixture was stirred at r.t. overnight. LCMS showed the reaction was completed. The reaction solution was concentrated under reduced pressure. The crude product was dissolved with anhydrous DCM (20 mL), to the resulting solution was added HATU (0.12 g, 0.31 mmol), DIEA (0.53 g, 0.46 mmol) and compound VII-IIA (0.054 g, 0.3 mmol). The reaction mixture was stirred at r.t. overnight. The mixture was diluted with water (20 mL) and extracted with EtOAc (50 mL×3). The combined organic layers were washed with brine, dried over sodium sulfate, and concentrated under reduced pressure. The residue was purified by Prep-HPLC to afford compound 402 (0.04 g, yield 34%) as a white solid. ¹H NMR (400 MHz, CDCl₃) δ 8.1 (m, 1 H), 7.5 (m, 7H), 7.3 (m, 1H), 7.0 (m, 1H), 5.6 (d, 2H), 5.3 (m, 2H), 4.2 (m, 2H), 3.7 (m, 2H), 3.5 (m, 6H), 2.9 (m, 2H), 2.2 (m, 2H), 2.0 (m, 7H), 1.9 (m, 3H), 1.0 (m, 12H). MS (ESI) m/z (M+H)⁺ 763.5.

### Example VIII-IIII

### Preparation of Compound 403

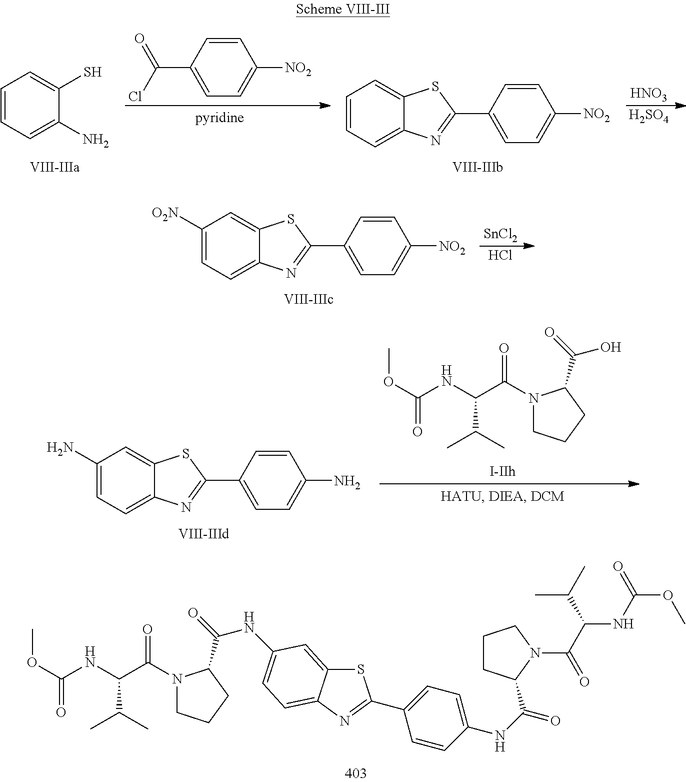

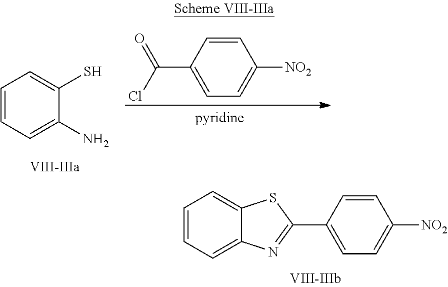

### General Procedure VIII-M

To a solution of 2-amino-benzenethiol (VIII-IIIa) (5 g, 40 mmol) in pyridine (30 mL) was added 4-nitro-benzoyl chloride (7.4 g, 40 mmol). The mixture was stirred at reflux for 2 hours. The reaction mixture was poured into ice-water (100 mL). The precipitate was filtered and washed with methanol (20 mL) to afford 2-(4-nitrophenyl)benzo[d]thiazole (VIII-IIIb) (6.6 g, yield 76%). MS (ESI) m/z (M+H)⁺ 257.

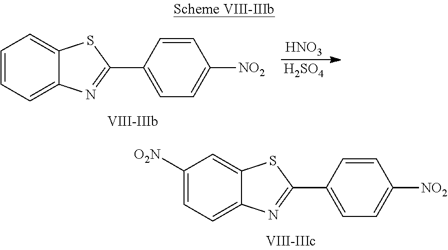

### General Procedure VIII-N

To a solution of 2-(4-nitrophenyl)benzo[d]thiazole (VIII-IIIb) (2.56 g, 10 mmol) in H₂SO₄ (conc. 10 mL) was added a mixture of HNO₃ and H₂SO₄ (15 mL, 2:1). The resulting mixture was heated at 80° C. overnight under nitrogen protection. TLC monitored the reaction. After completion of the reaction, the mixture was poured into water, and the precipitate was washed with water (10 mL), collected and dried to afford 6-nitro-2-(4-nitrophenyl)benzo[d]thiazole (VIII-IIIc) (2.5 g, yield: 83%). MS (ESI) m/z (M+H)⁺ 302.

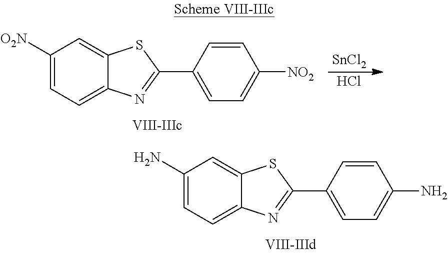

### General Procedure VIII-O

To a suspension of 6-nitro-2-(4-nitrophenyl)benzo[d]thiazole (0.9 g, 3 mmol) in methanol (10 mL) and HCl (conc. 5 mL) was added SnCl₂ (3.8 g, 20 mmol). The mixture was heated to reflux for 15 min., after which it was concentrated in vacuum. The residual was neutralized with aqueous K₂CO₃, and extract with DCM (15 mL×2). The organic layer was separated, dried over Na₂SO₄ and concentrated under vacuum to afford 2-(4-aminophenyl)benzo[d]thiazol-6-amine (VIII-IIId) (0.35 g, yield 49%), which was used to next step directly.

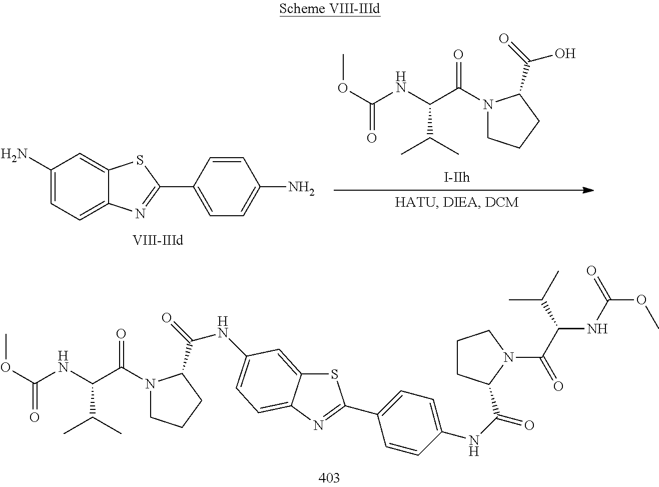

### General Procedure

To a solution of compound I-IIh (0.27 g, 1 mmol), HATU (0.38 g, 1 mmol) and DIEA (0.5 mL) was added 2-(4-aminophenyl)benzo[d]thiazol-6-amine (VIII-IIId) (72 mg, 0.3 mmol). The mixture was stirred at r.t. for 1 h. The mixture was washed with aqueous K₂CO₃ (2 mL). The organic layer was separated and concentrated under reduced pressure. The residue was purified by Prep-HPLC to afford compound 403 (190 mg, yield 84.8%). MS (ESI) m/z (M+H)⁺ 750.3.

### Example VIII-X

### Preparation of Compounds 402 and 410

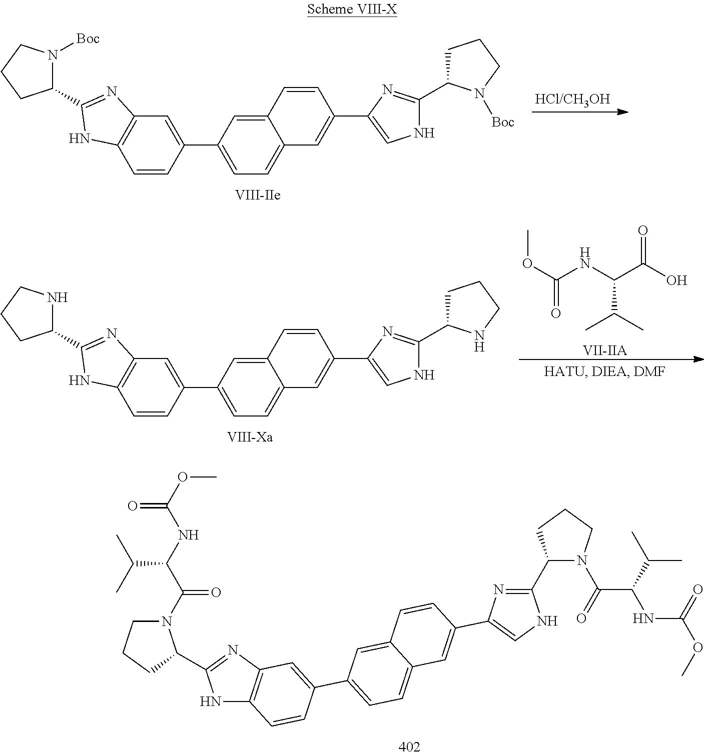

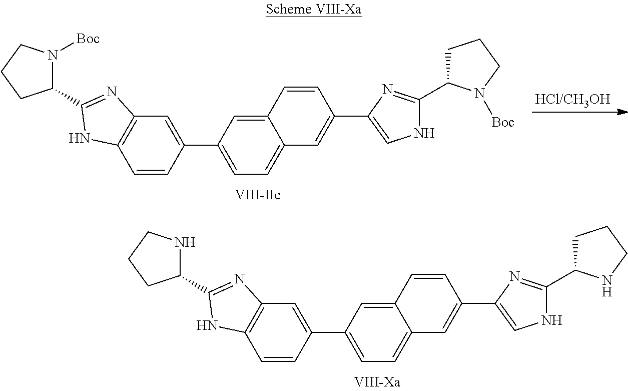

### General Procedure VIII-AU

Compound VIII-IIe (2.97 g, 0.11 mmol) was added into HCl/CH₃OH (40 mL, 4M). Then the mixture was stirred at room temperature for 2-3 hr. After the reaction was completed, the mixture was concentrated in vacuum to give compound VIII-Xa (2.40 g, yield: 92%).

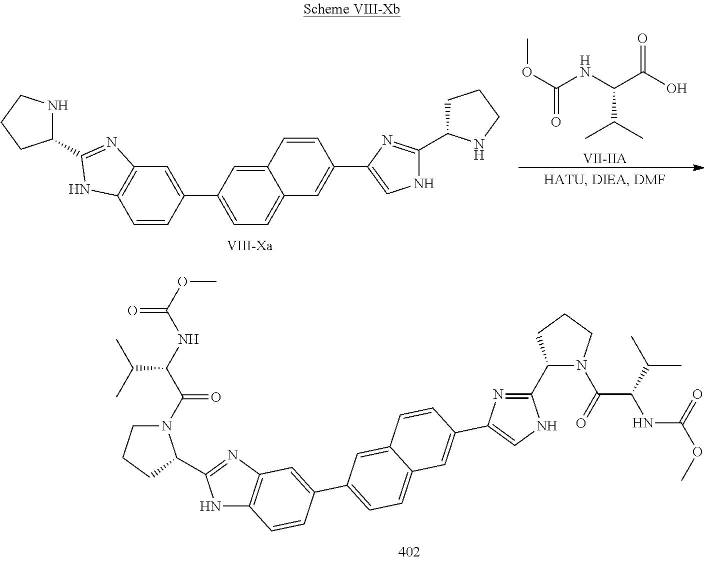

### General Procedure VIII-AV

To a mixture of compound VIII-Xa (2.10 g, 4.68 mmol), compound VII-IIA (1.64 g, 4.68 mmol) and DIPEA (3.63 g, 28.13 mmol) in DMF (50 mL) was added HATU (3.56 g, 4.68 mmol). The resulting mixture was stirred at room temperature. LCMS indicated the disappearance of compound VIII-Xa. The mixture was purified by Pre-HPLC to give compound 402 (1.01 g, yield: 53%). ¹H NMR (400 MHz, CDCl₃) δ 7.28-8.01 (m, 9 H), 7.0 (m, 1H), 5.30-5.60 (m, 4H), 4.29-4.33 (m, 2H), 3.79-3.83 (m, 2H), 3.65-3.72 (m, 2H), 3.64 (s, 6H), 2.9 (m, 2H), 1.97-2.35 (m, 10H), 0.83-0.85 (m, 12H). MS (ESI) m/z (M+H)⁺ 763.4.

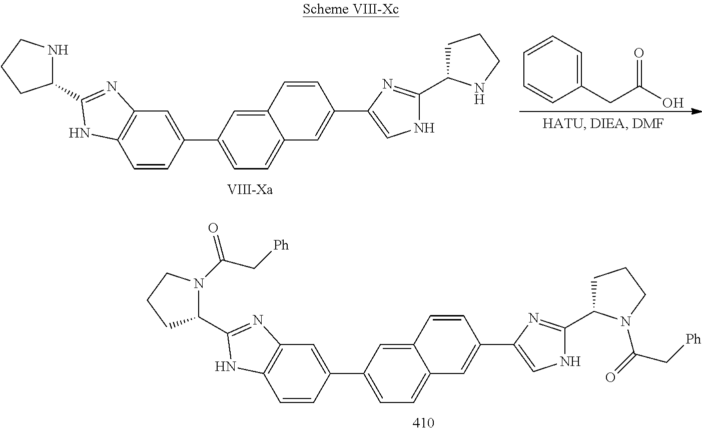

### General Procedure VIII-AW

Compound 410 was prepared following general procedure VIII-AV (17 mg, yield 22%). ¹H NMR: (400 MHz, CDCl₃) δ 7.21-7.50 (m, 9H), 6.86-7.12 (m, 12H), 5.23-5.44 (m, 2H), 4.07-4.17 (m, 2H), 3.69-3.91 (m, 5H), 3.56 (br, 3H), 2.39 (br, 5H), 2.03 (br, 5H). MS (ESI) m/z (M+H)⁺ 685.3.

### Example VIII-XI

### Preparation of Compounds 411-414

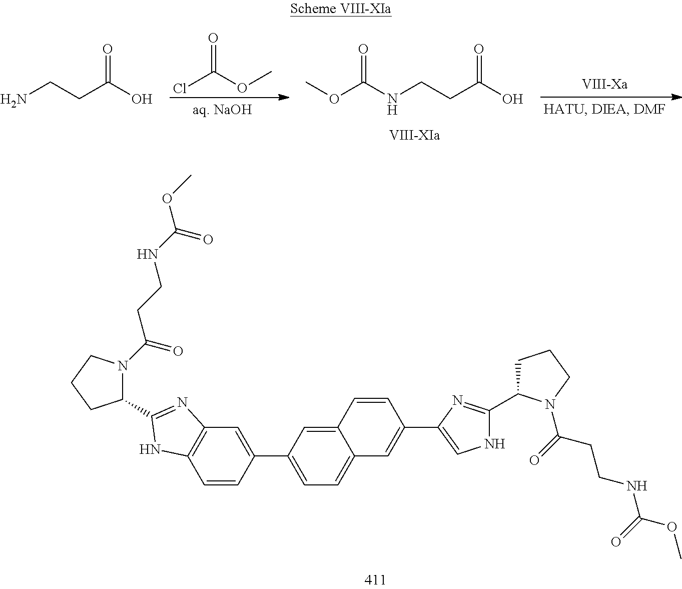

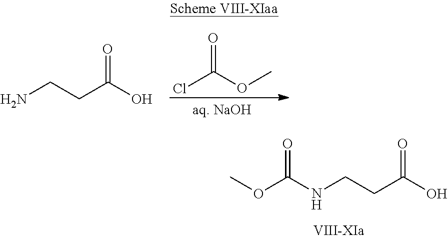

### General Procedure VIII-AX

A solution of 3-aminopropionic acid (2 g, 22.47 mmol) in 22 mL of 1N sodium hydroxide solution were cooled to 10° C. Methyl chloroformate (2.12 g, 22.47 mmol) and 11 mL of a 2N NaOH solution were added simultaneously to the cooled mixture. After stirring for 16 h at r.t., the mixture treated with 1N aqueous HCl until a pH of 2 was reached. Subsequently, the mixture was extracted with ethyl acetate (100 mL×3). The extracts were combined, dried over sodium sulfate, filtered and concentrated to yield compound VIII-XIa (1.2 g, yield: 36%) as a white solid.

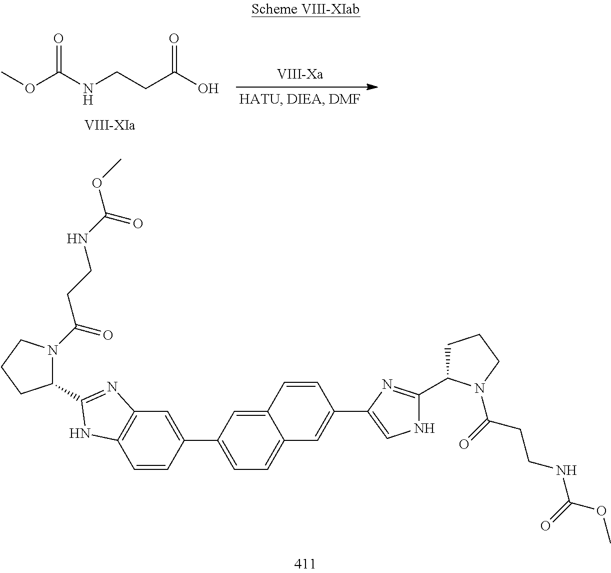

### General Procedure VIII-AY

Compound 411 was prepared following general procedure VIII-AV (36 mg, yield 37%). ¹H NMR: (400 MHz, CDCl₃) δ 7.38-7.69 (m, 7H), 6.93-7.12 (m, 3H), 5.76 (br, 2H), 5.45-5.51 (m, 2H), 3.91-4.01 (m, 2H), 3.56-3.57 (br, 2H), 3.42-3.47 (m, 12H), 2.51-2.77 (m, 12H). MS (ESI) m/z (M+H)⁺ 707.3

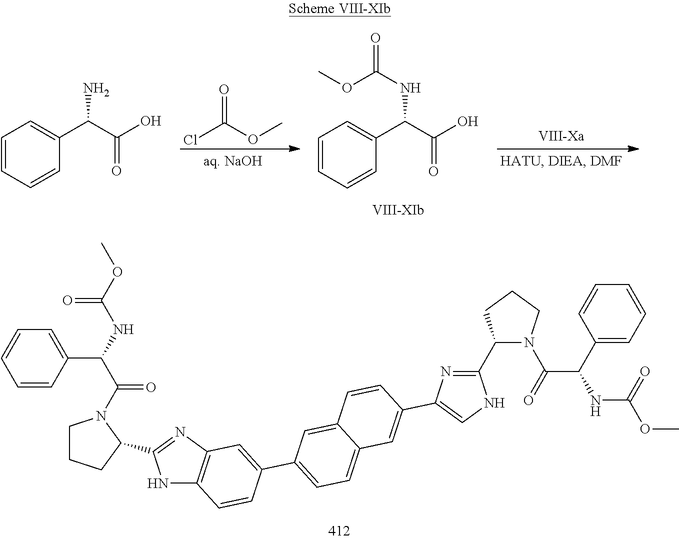

### General Procedure VIII-AZ

Compound 412 was prepared following general procedure VIII-AX and general procedure VIII-AV (30 mg, yield 31%). ¹H NMR: (400 MHz, CDCl₃) δ 7.31-7.67 (m, 15H), 7.12-7.19 (m, 5H), 5.50-5.56 (m, 4H), 3.86-4.07 (m, 4H), 3.54-3.66 (m, 5H), 3.12-3.34 (m, 5H), 2.10-2.47 (m, 8H), MS (ESI). m/z (M+H)⁺ 831.4.

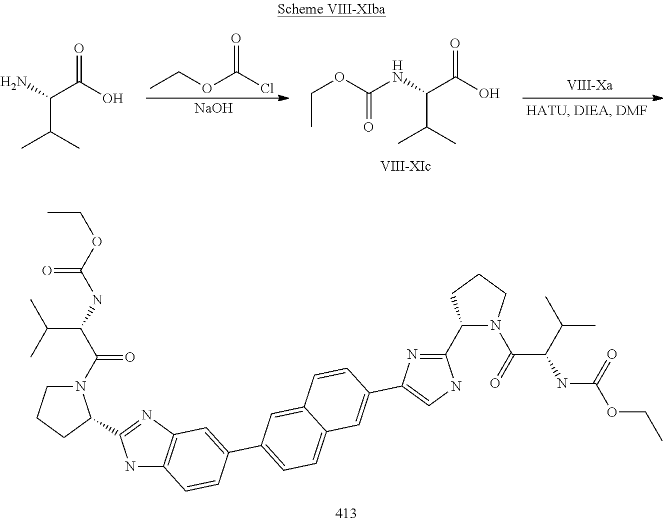

### General Procedure VIII-BA

Compound 413 was prepared following general procedure VIII-AX and general procedure VIII-AV (40 mg, yield 39%).

MS (ESI) m/z (M+H)⁺: 791.4.

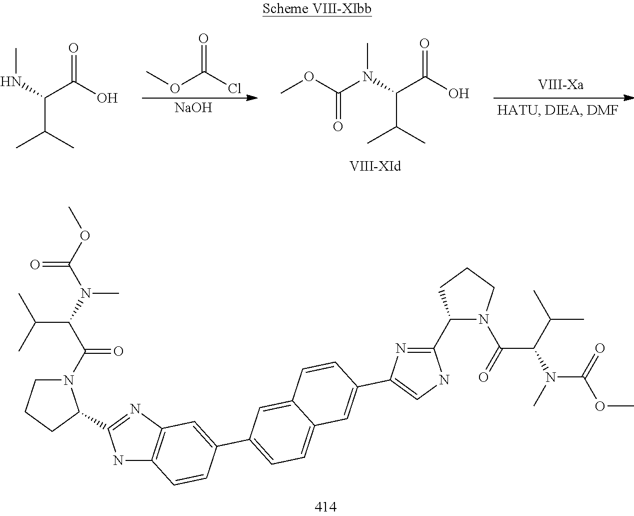

### General Procedure

Compound 414 was prepared following general procedure VIII-AX and general procedure VIII-AV (20 mg, yield 24%). ¹H NMR (400 MHz, CDCl₃) δ7.41-8.18 (m, 7H), 6.73-7.06 (m, 3H), 5.37-5.60 (m, 2H), 4.54-4.72 (m, 2H), 4.19-4.21 (m, 2H), 3.71-3.90 (m, 7H), 3.48-3.57 (m, 1H), 2.40-2.69 (m, 9H), 2.21-2.34 (m, 6H), 0.77-0.88 (m, 12H). MS (ESI) m/z (M+H)⁺: 791.4.

### Example VIII-XII

### Preparation of Compounds 415 and 416

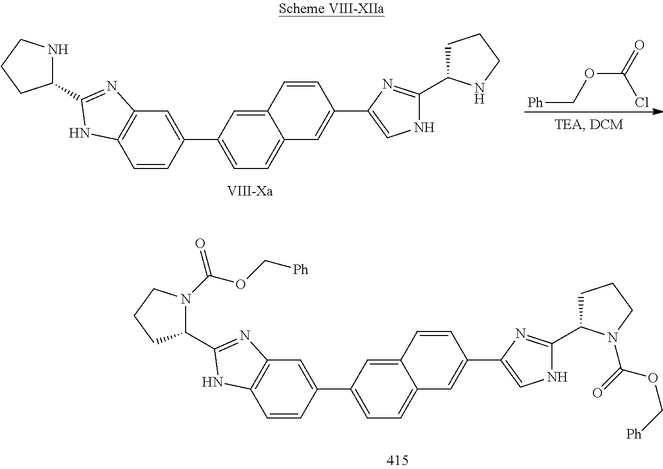

### General Procedure VIII-BC

To a stirred solution of compound VIII-Xa (70 mg, 0.16 mmol) and TEA (87 mg, 0.86 mmol) in DCM was added dropwise CbzCl (53 mg, 0.31 mmol) at 0° C. under argon. After addition, the solution was stirred at 0° C. for 0.5 hour, then warmed slowly to the room temperature, and stirred for another 3 hours. The mixture concentrated and purified by prep-HPLC to afford compound 415 (5.1 mg, yield 6%). MS (ESI) m/z (M+H)⁺ 717.3.

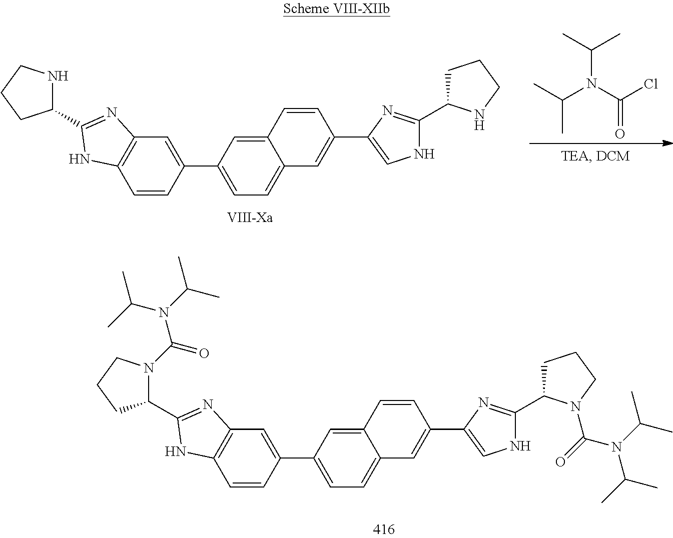

### General Procedure VIII-BD

Compound 416 was prepared following general procedure VIII-BC (45 mg, yield 43%). ¹H NMR (400 MHz, CDCl₃): δ7.35-7.70 (m, 5H), 6.42-7.02 (m, 5H), 5.49 (m, 2H), 3.91-4.03 (m, 6H), 3.42-3.49 (m, 2H), 2.29 (m, 6H), 2.05 (m, 2H), 1.24-1.30 (m, 12H). MS (ESI) m/z (M+H)⁺: 703.3.

### Example VIII-XIII

### Preparation of Compound 417

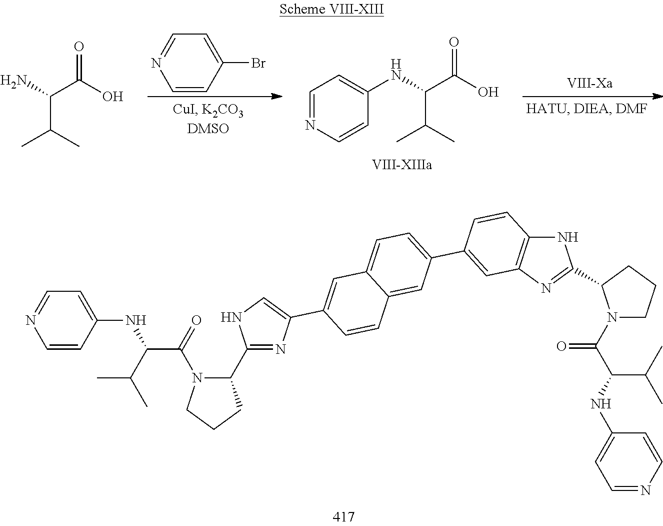

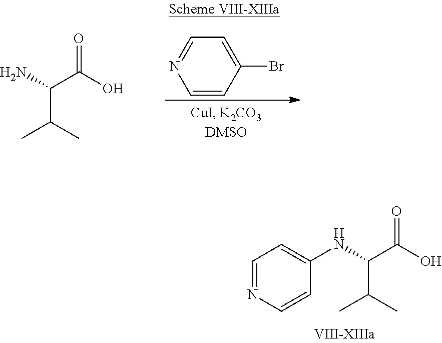

### General Procedure VIII-BE

A mixture of L-valine (2.0 g, 17 mmol), 4-bromopyridine (5.36 g, 34 mmol), K₂CO₃ (4.2 g, 34 mmol) and CuI (0.3 g, 1.7 mmol) in DMSO (20 mL) was stirred at 100° C. for 12 h. The reaction mixture was cooled to r.t, poured into water (150 mL) and extracted with EtOAc (100 mL×2). The organic layers were separated, dried and concentrated. The residue was purified by prep-HPLC to afford compound VIII-XIIIa (1.0 g, yield 31%).

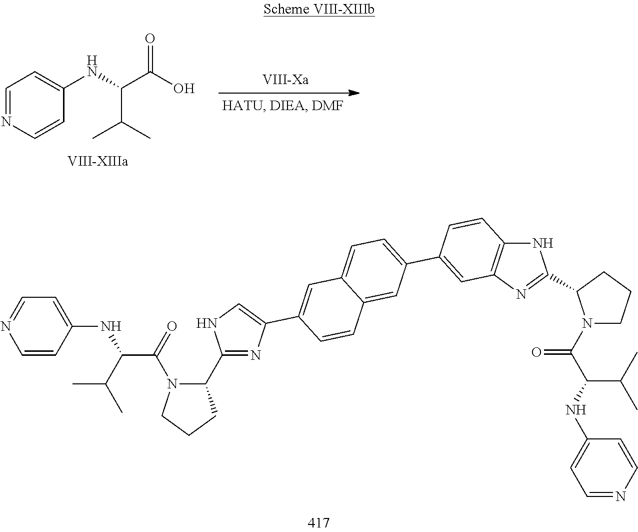

### General Procedure VIII-BF

To a stirred mixture of compound VIII-Xa (50 mg, 0.11 mmol), HATU (125 mg, 0.33 mmol) and DIEA (43 mg, 0.33 mmol) in DCM was added compound VIII-XIIIa (64 mg, 0.33 mmol), the mixture was stirred at r.t. for 1 h. The mixture was diluted with DCM, washed with water and brine, separated the organic layer, dried, filtered and concentrated. The residue was purified by prep-HPLC to afford compound 417 (33.6 mg, yield 38%). ¹H NMR (400 MHz, CDCl₃) δ 8.23-8.09 (m, 4H), 8.06-7.68 (m, 6H), 7.67-7.47 (m, 2H), 7.42-7.27 (m, 2H), 6.55-6.39 (m, 4H), 5.50-5.22 (m, 2H), 5.12-4.58 (m, 2H), 4.18-4.07 (m, 2H), 3.86-3.67 (m, 4H), 3.20-2.88 (m, 2H), 2.68-2.36 (m, 2H), 2.36-2.04 (m, 6H), 1.35-0.87 (m, 12H). MS (ESI) m/z (M+H)⁺ 801.5.

### Example VIII-XIV

### Preparation of Compounds 418 and 419

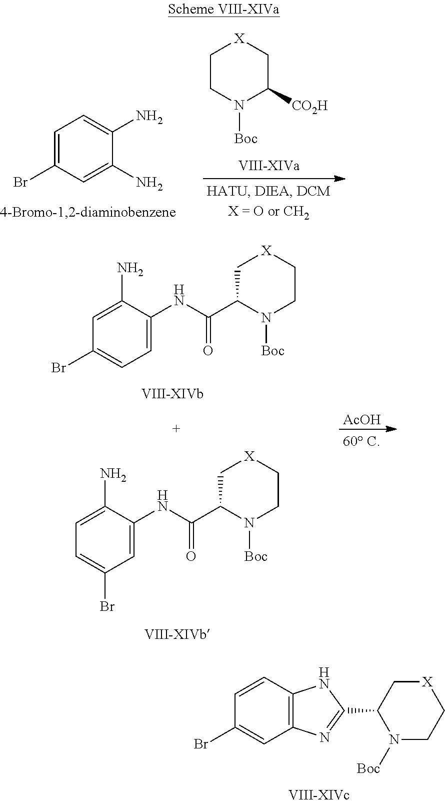

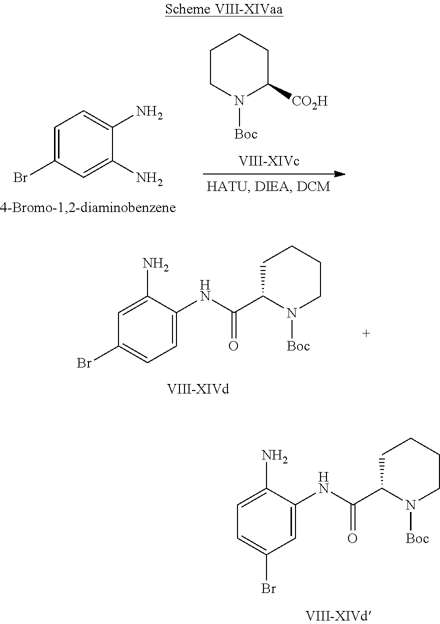

### General Procedure VIII-BG

To a stirring solution of 4-Bromo-1,2-diaminobenzene (0.5 g, 2.7 mmol), compound VIII-XIVc (0.65 g, 2.7 mmol), and DIEA (1.35 mL, 8.1 mmol) in CH₂Cl₂ (60 mL) was added HATU (1.1 g, 2.7 mmol, portionwise). After 14 hrs, the mixture was washed with saturated aq. NaHCO₃ solution, and the aqueous layer was extracted with CH₂Cl₂ (30 mL×3). The combined extracts were washed with brine and dried over anhydrous NaSO₄. The solvent was removed under the reduced pressure to obtain compound VIII-XIVd or VIII-XIVd′ (0.8 g) used directly in the next step.

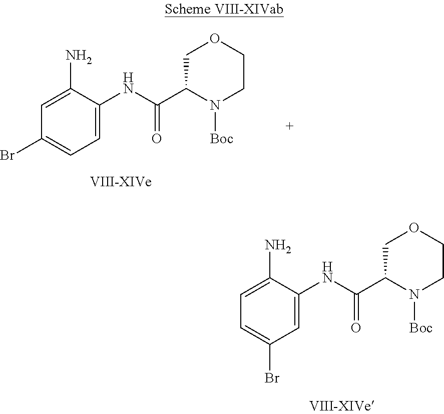

The preparation of compound VIII-XIVe or VIII-XIVe′ followed general procedure VIII-BG.

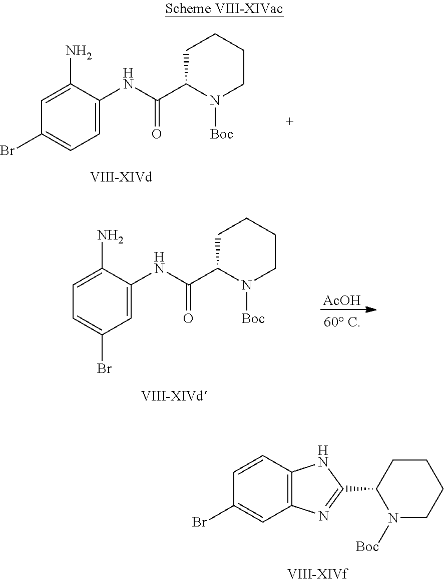

### General Procedure VIII-BH

Compound VIII-XIVd or VIII-XIVd′ (0.8 g, 2 mmol) was taken up in glacial acetic acid (30 mL) and heated at 60° C. for 3 hrs. The solvent was removed in vacuo and the residue was taken up in EtOAc, washed with saturated aq. NaHCO₃ solution (adjust with 1N NaOH until pH=9), brine, and dried over anhydrous Na₂SO₄, filtered, and concentrated. The residue was obtained and purified by flash chromatography on silica gel to afford compound VIII-XIVf (0.7 g, yield 68% over two steps) as a yellow foam. ¹H NMR (300 MHz, CDCl₃): δ 9.72 (br, 1H), 7.70-7.32 (m, 3H), 5.53 (s, 1H), 4.13 (t, 1H), 2.73-2.59 (m, 2H), 2.12-1.72 (m, 5H), 1.57 (s, 9H).

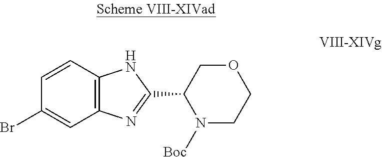

The preparation of compound VIII-XIVg (300 mg, yield 29% over two steps) followed general procedure VIII-BH.

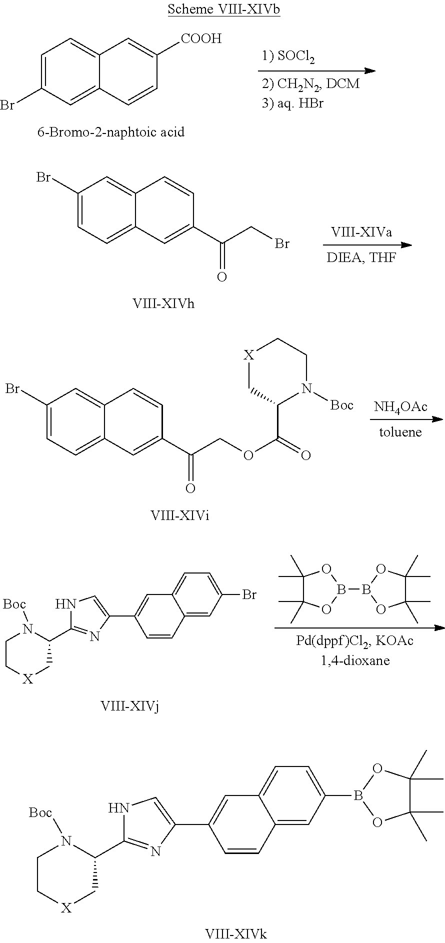

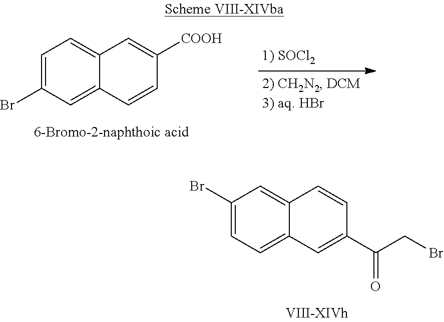

### General Procedure VIII-BI

A mixture of 6-bromo-2-naphthoic acid (2 g, 7.96 mmol), SOCl₂ (20 mL) (adding two drops of DMF) was refluxed for 2 hrs. The excess of SOCl₂ was removed under reduced pressure. The residue was co-evaporated with toluene (5 mL) for three times. The residue was dissolved in CH₂Cl₂ (5 mL) and the resulting solution was added dropwise to a solution of CH₂N₂ in ether (0.7 M, 57 mL, 39.8 mmol) at −10° C. The reaction mixture was stirred at 0° C. for 1 h. The reaction mixture was cooled to −10° C. again, to this solution was added dropwise aqueous HBr (48%, 4.7 mL, 39.8 mmol). The reaction mixture was stirred at the same temperature for 1 h, washed with saturated aq. NaHCO₃ and brine. The organic phase was dried over anhydrous Na₂SO₄, and concentrated to give compound VIII-XIVh as a pale yellow solid (2.1 g, yield 91%). ¹H NMR (400 MHz, CDCl₃): δ 8.42 (s, 1H), 8.00-7.98 (m, 2H), 7.80-7.76 (m, 2H), 7.61-7.58 (m, 1H), 4.49 (s, 2H).

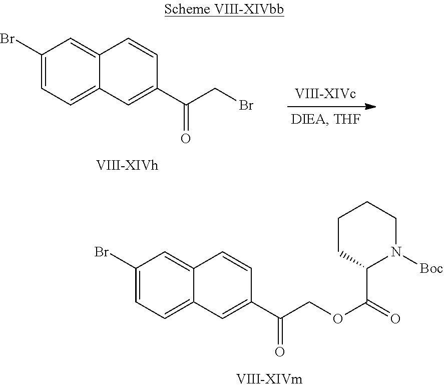

### General Procedure VIII-BJ

Diisopropylethylamine (0.53 mL) and compound VIII-XIVc (0.5 g, 2.17 mmol) were added to a suspension of compound VIII-XIVh (0.5 g, 1.53 mmol) in THF (20 mL). The resulting mixture was stirred at r.t. overnight. After addition of brine, the layers were separated, and the organic layer was dried over anhydrous Na₂SO₄, and concentrated. The residue was purified by column chromatography on silica gel to afford compound VIII-XIVm as a pale yellow solid (590 mg, yield 80%).

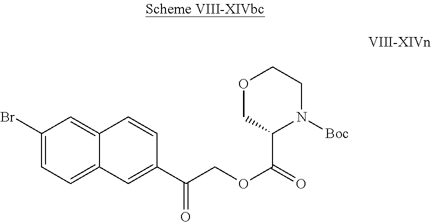

The preparation of compound VIII-XIVn (400 mg, yield 91%) followed general procedure VIII-BJ.

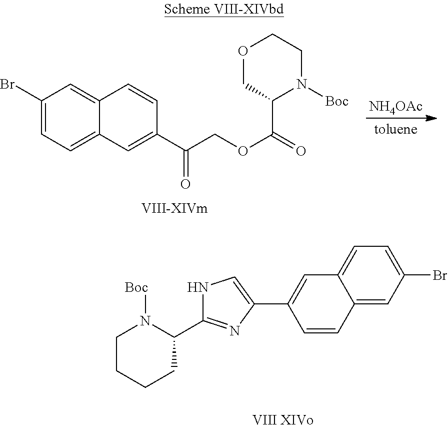

### General Procedure VIII-BK

To a solution of compound VIII-XIVm (700 mg, 1.47 mmol) in toluene (20 mL) was treated with ammonium acetate (2.26 g, 29.3 mmol), and reaction mixture was heated overnight at 100° C. The solvent was removed under reduced pressure to dryness; the residue was purified by column chromatography on silica gel to provide compound VIII-XIVo as a yellow solid (436 mg, yield 65%).

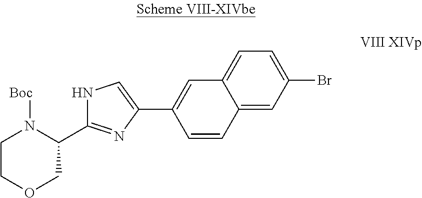

The preparation of VIII-XIVp (300 mg, yield 78%) followed general procedure VIII-BK.

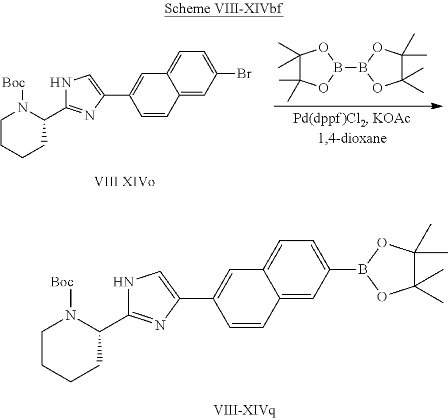

### General Procedure VIII-BL

A solution of compound VIII-XIVo (260 mg, 0.57 mmol), bis(pinacolato)diboron (219 mg, 0.85 mmol), Pd(dppf)Cl₂ (47.58 mg, 0.057 mmol), and KOAc (170.7 mg, 1.7 mmol) in degassed 1,4-dioxane (15 mL) was stirred overnight at 80° C. under N₂ protection. The reaction was cooled down to r.t. and diluted with water (10 mL), and the resulting mixture was extracted with EtOAc. The combined organic layer was dried over anhydrous Na₂SO₄, and concentrated to give a residue, the residue then purified by column chromatography on silica gel to give compound VIII-XIVq as a yellow solid (200 mg, yield 70%).

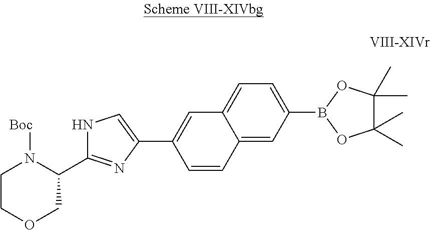

The preparation of compound VIII-XIVr (80 mg, yield 48%) followed general procedure VIII-BL.

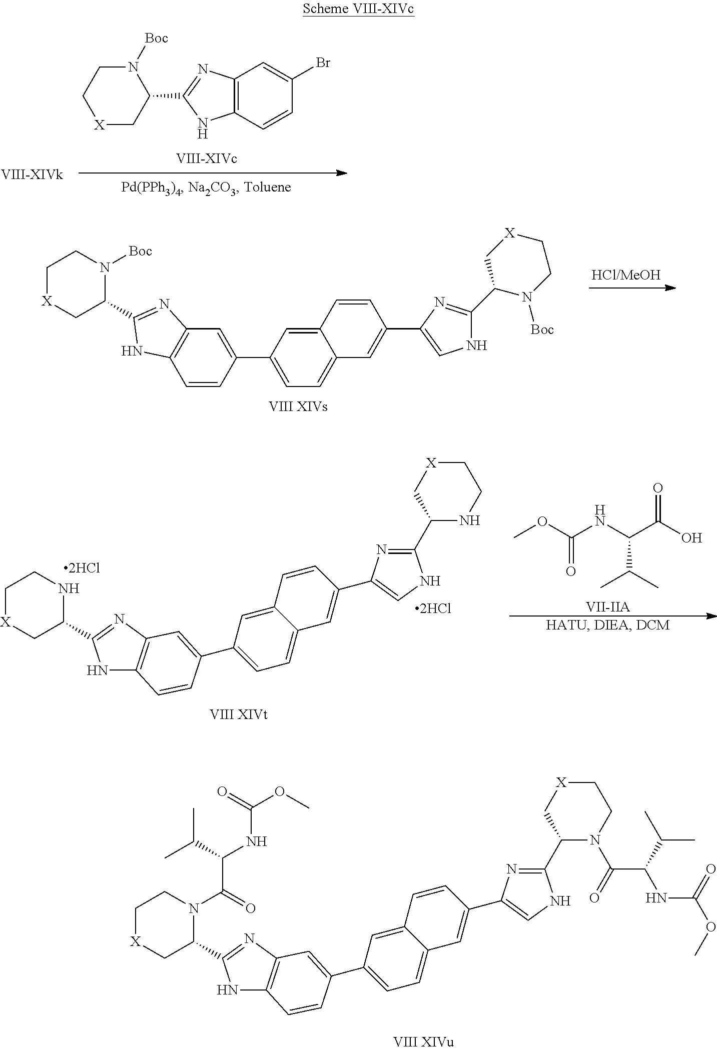

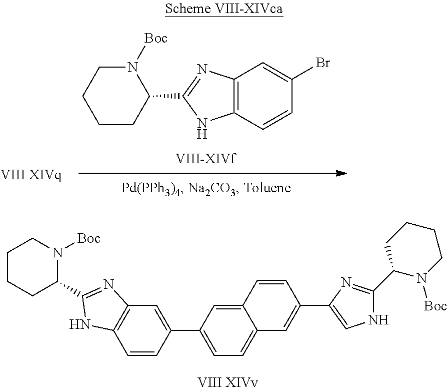

### General Procedure VIII-BM

A mixture of compound VIII-XIVq (175 mg, 0.35 mmol), compound VIII-XIVf (115 mg, 0.3 mmol), Pd(dppf)Cl₂ (40 g, 0.05 mmol) and Na₂CO₃ (85 mg, 0.8 mmol) was dissolved in toluene (20 mL) and H₂O (2 mL). The mixture was purged with N₂ and heated at 90° C. for 12 hrs under N₂ protection. After cooling, the mixture was poured into water and extracted with EtOAc. The organic layer was washed with brine and dried with anhydrous Na₂SO₄. After the solvent was evaporated, the residue was purified by column chromatography on silica gel to provide compound VIII XIVv as a pale yellow solid (120 mg, yield 59%).

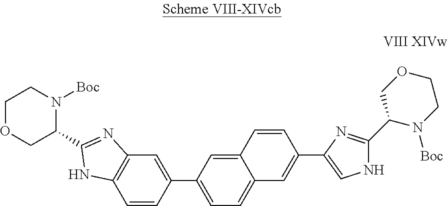

The preparation of compound VIII XIVw (40 mg, yield 60%) followed general procedure VIII-BM.

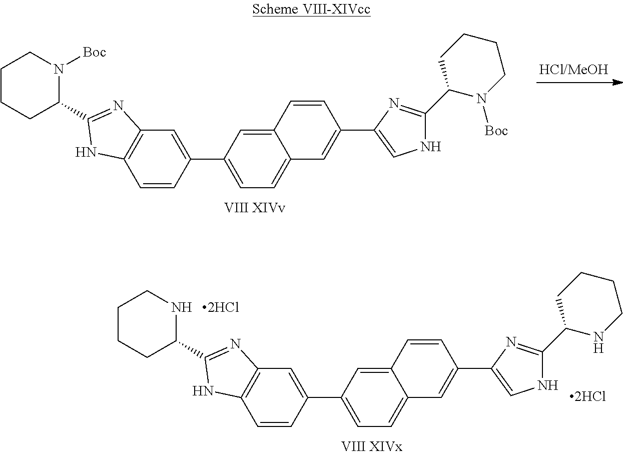

### General Procedure VIII-BN

Aqueous hydrochloric acid (6M, 3 mL) was added to a suspension of compound VIII XIVv (240 mg, 0.35 mmol) in methanol (20 mL). The resulting mixture was stirred overnight at r.t. and concentrated to dryness to yield compound VIII XIVx as a yellow solid (200 mg, yield 90%).

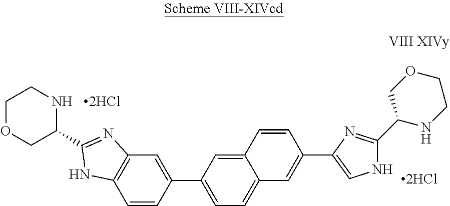

The preparation of VIII XIVy (40 mg, yield 100%) followed general procedure VIII-BN.

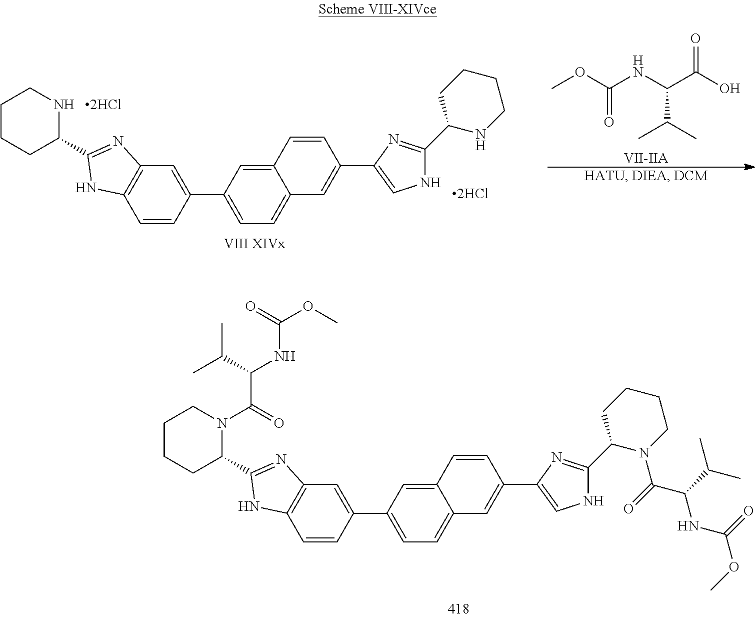

### General Procedure VIII-BO

To a solution of compound VIII XIVx (200 mg, 0.32 mmol) in anhydrous CH₂Cl₂ (20 mL), compound VII-IIA (124 mg, 0.64 mmol) and DIPEA (0.47 mL, 2.57 mmol) were added, then HATU (269 mg, 0.64 mmol) was added under the protection of N₂. The resulting mixture was stirred at r.t. overnight. The reaction mixture was poured into water (10 mL), and extracted with dichloromethane (5 mL×3). The combined extracts were washed with brine and dried over anhydrous Na₂SO₄. The solvent were removed under the reduced pressure to obtain residue. The residue was purified by Prep-HPLC to give compound 418 as a white solid (100 mg, yield 40%). MS (ESI) m/z [M+H]⁺ 791.5. ¹H NMR (400 MHz, CDCl₃): δ 11.83 (br, 1H), 8.25-7.47 (m, 10H), 6.00-5.35 (m, 4H), 4.44 (t, 2H), 4.63 (s, 2H), 3.82-3.69 (m, 8H), 2.97-2.05 (m, 15H), 1.12 (s, 12H).

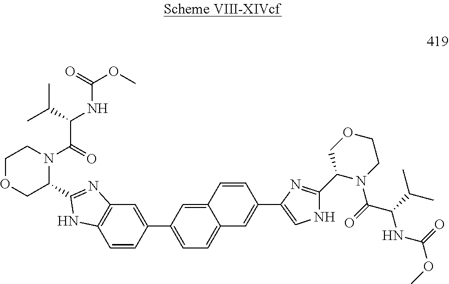

The preparation of compound 419 followed general procedure VIII-BO. (6.7 mg, yield 13%). MS (ESI) m/z [M+H]⁺ 795.

### Example VIII-XV

### Preparation of Compound 420

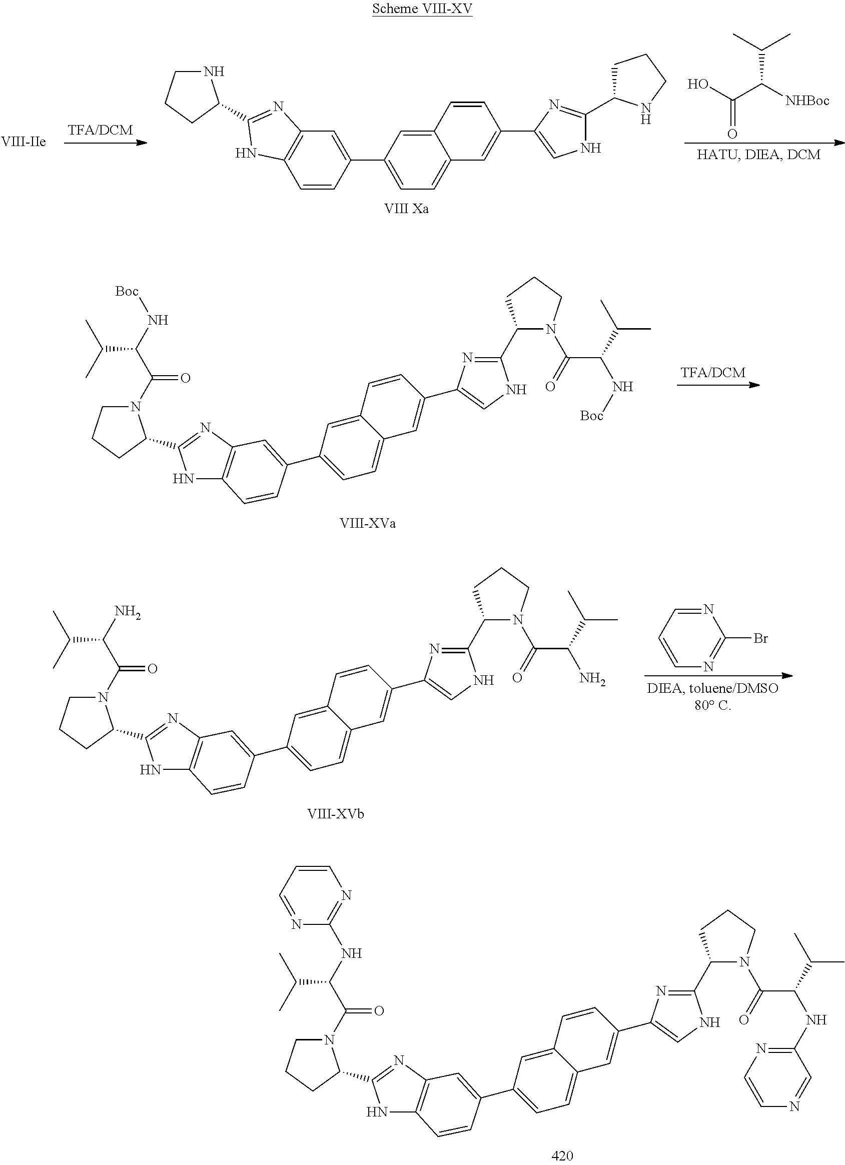

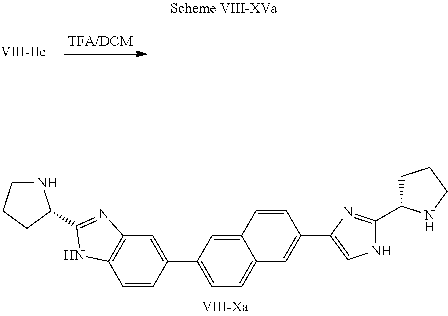

### General Procedure VIII-BP

To a stirred solution of compound VIII-IIe (500 mg, 0.77 mmol) in DCM (5 mL) was added TFA (3 mL), the mixture was stirred at r.t. for 30 min. The mixture was concentrated under reduced pressure to obtain a residue, it was dissolved in EtOAc (100 mL) and washed with aq. NaHCO₃, the organic layer was dried over Na₂SO₄ and concentrated under reduced pressure to provide compound VIII-Xa (300 mg, yield 87%). MS (ESI) m/z (M+H)⁺ 449.

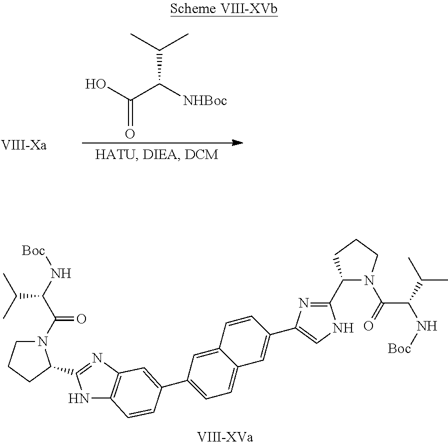

### General Procedure VIII-BQ

A mixture of compound VIII-Xa (300 mg, 0.67 mmol), N-Boc-L-valine (434 mg, 2 mmol), HATU (760 mg, 2 mmol) and DIEA (260 mg, 2 mmol) in DCM was stirred at r.t. for 1 h. The mixture was diluted with DCM, washed with water and brine. The organic layer was separated, dried over Na₂SO₄ and concentrated under reduced pressure. The residue was purified by Prep-HPLC to afford compound VIII-XVa (400 mg, yield 70%).

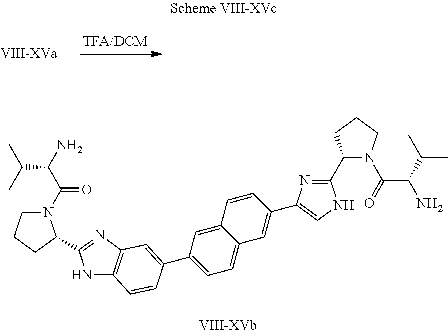

### General Procedure VIII-BR

To a stirred solution of compound VIII-XVa (400 mg, 0.62 mmol) in DCM (5 mL) was added TFA (2 mL), the mixture was stirred at r.t. for 30 min. The mixture was concentrated under reduced pressure to obtain a residue, which was dissolved in EtOAc (100 mL) and washed with aq. NaHCO₃, the organic layer was dried over Na₂SO₄ and concentrated under reduced pressure to provide compound VIII-XVb (220 mg, yield 80%). MS (ESI) m/z: 647 (M+H).

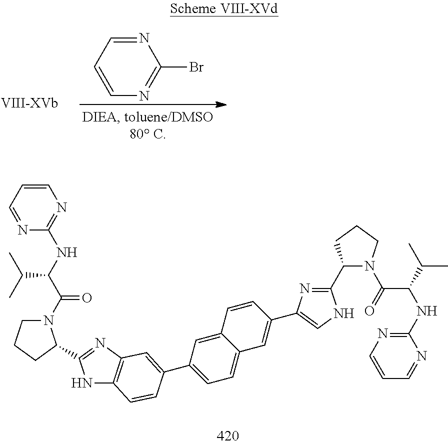

### General Procedure VIII-BS

A mixture of compound VIII-XVb (150 mg, 0.23 mmol), 2-bromopyrimidine (218 mg, 1.38 mmol) and DIEA (178 mg, 1.38 mmol) in toluene/DMSO (4:1, 3 mL) was stirred at 80° C. for 16 hrs. The mixture was diluted with EtOAc, washed with water and brine, separated the organic layer, dried over Na₂SO₄, filtered and concentrated. The residue was purified by prep-HPLC to afford 420 (15 mg, yield 8%). ¹H NMR (400 MHz, CD₃OD) δ 8.30-8.29 (m, 3H), 8.21-8.06 (m, 2H), 7.95-7.75 (m, 6H), 7.73-7.28 (m, 4 H), 6.65 (m, 1H), 5.52-5.17 (m, 2H), 4.72-4.54 (m, 2H), 4.34-4.18 (m, 1H), 4.04-3.86 (m, 2H), 3.71-3.52 (m, 1H), 1.94-2.58 (m, 10H), 1.09-0.93 (m, 12H). MS (ESI) m/z: 803.5 (M+H).

### Section IX

### Example IX-I

### Compound 500 can be Prepared According to the Following Scheme

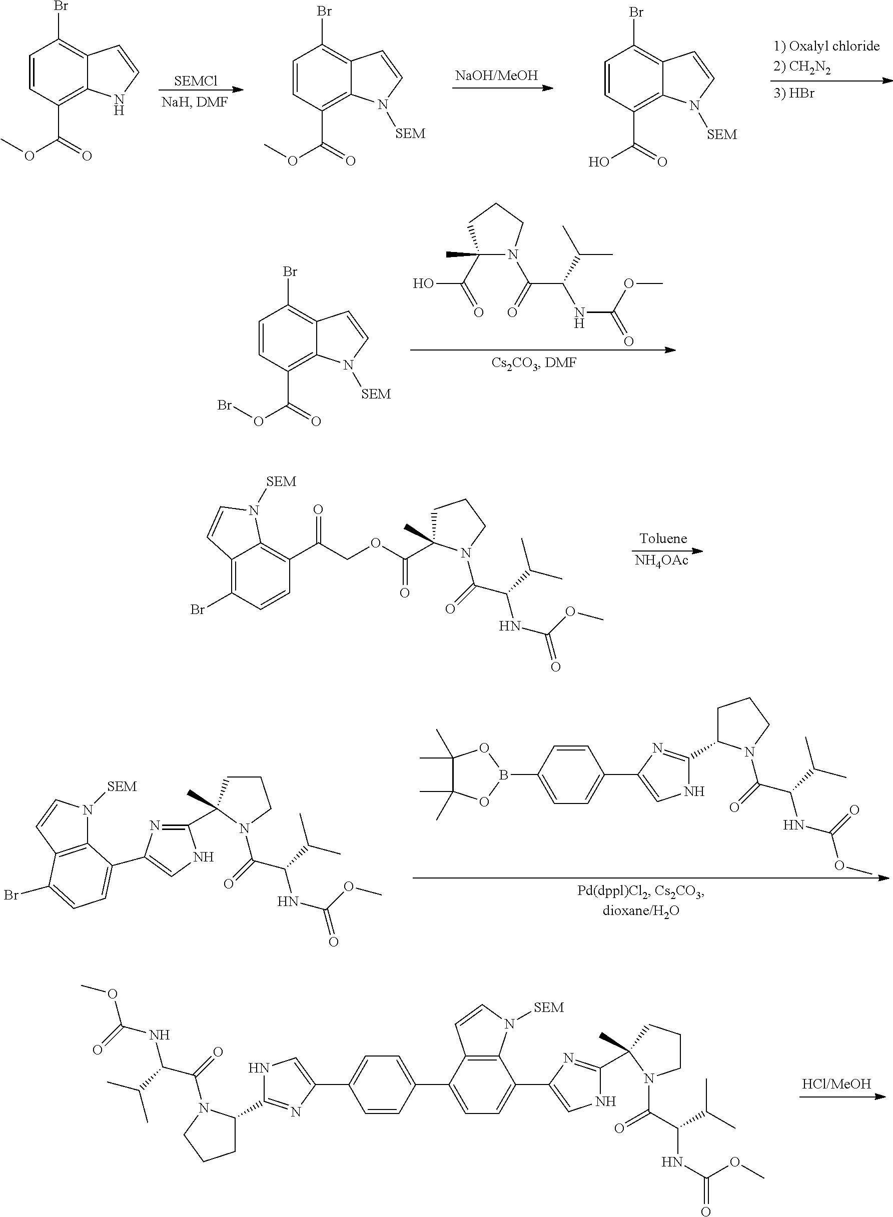

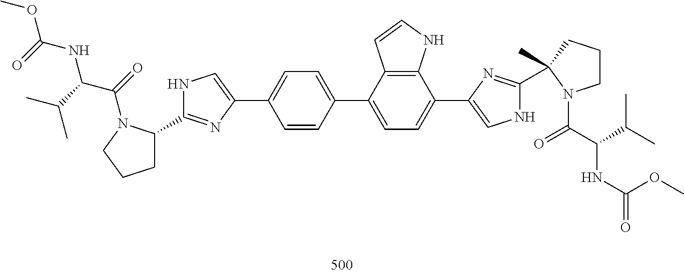

### Example IX-II

### Compound 501 can be Prepared According to the Following Scheme

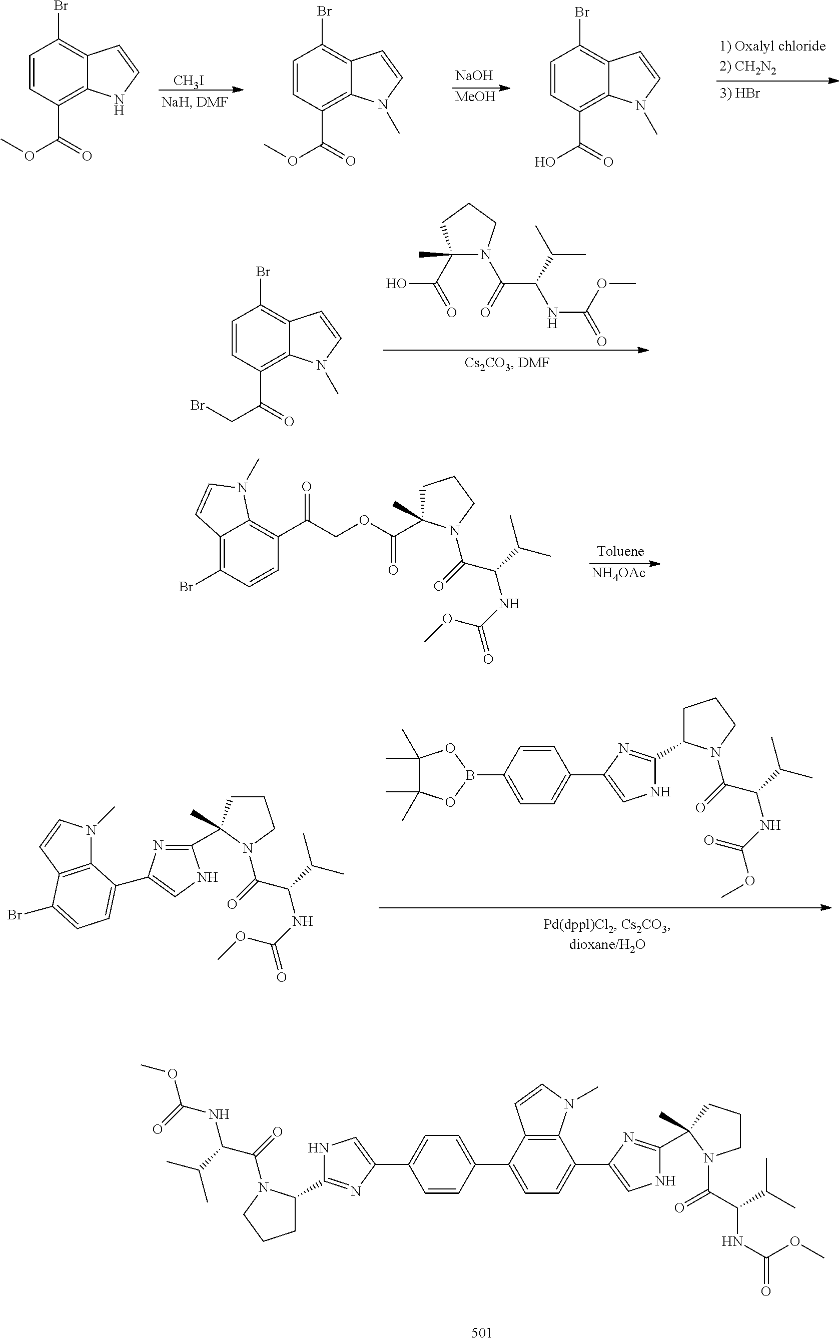

### Example IX-III

### Preparation of Compound 502

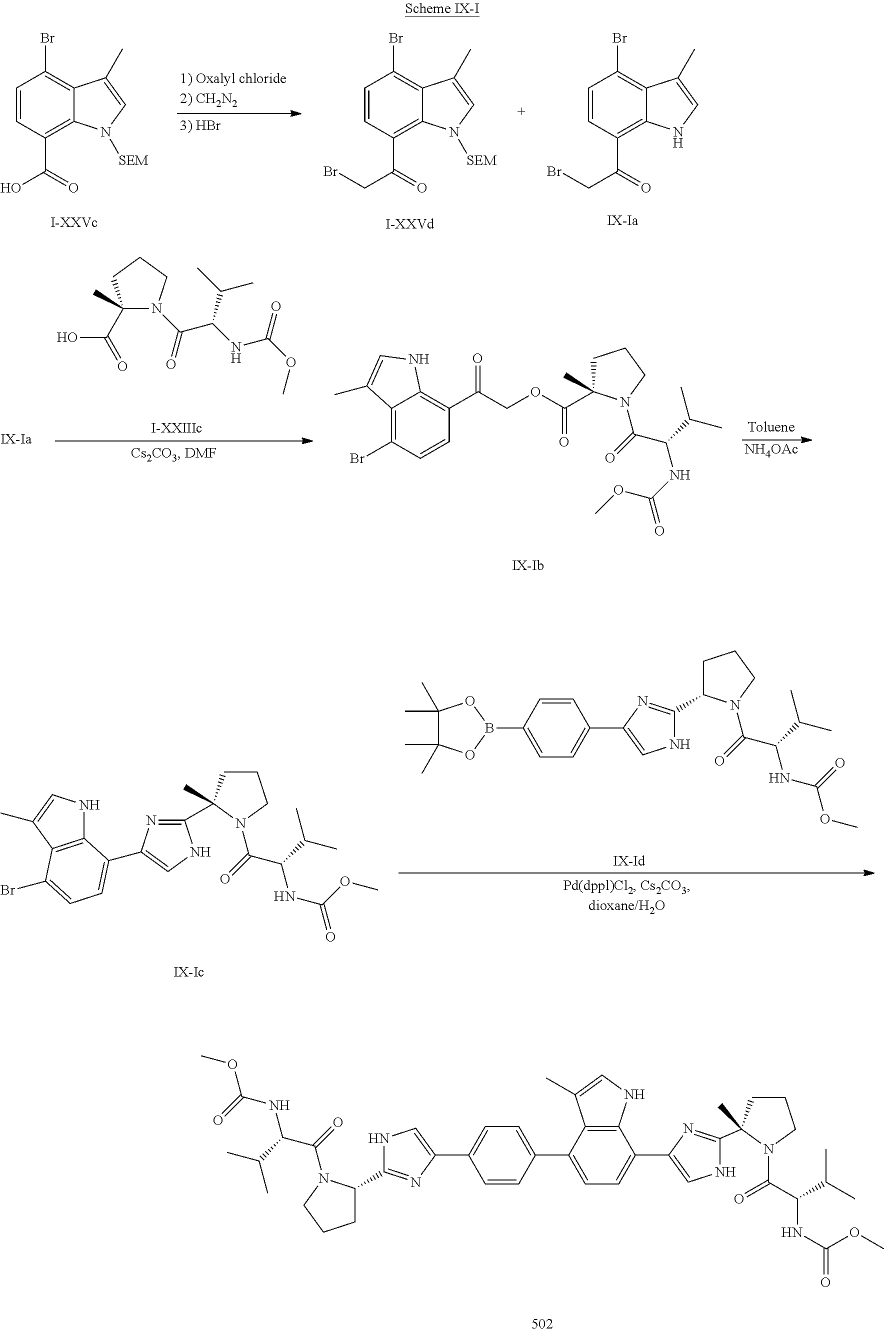

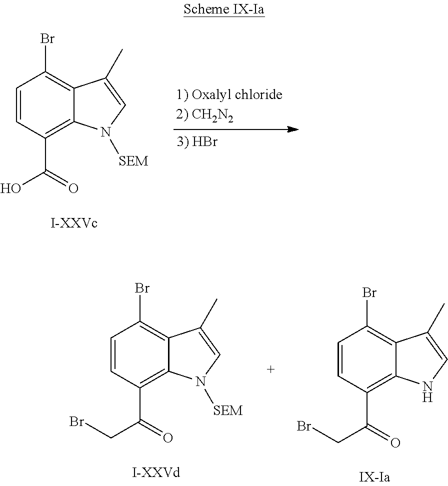

### General Procedure IX-A

To a solution of compound I-XXVc (1.8 g, 4.7 mmol) in 20 mL of dry DCM was added oxalyl chloride (1.2 g, 9.4 mmol) dropwise at 0° C. under nitrogen protection. The mixture was stirred at room temperature for 2 hours. Subsequently, the solvent was removed and the residue was dissolved with 10 mL of dry DCM. The resulting solution was added dropwise to a solution of diazomethane (24 mmol) in 40 mL of Et₂O at −10° C. under nitrogen. The mixture was stirred at room temperature for 2 hours. The reaction was then cooled down and 20 mL of aq. HBr added dropwise. The resulting mixture was stirred for 1 hour. The mixture was then washed with aq. NaHCO₃ and brine. The organic layer was separated dried over anhydrous Na₂SO₄, concentrated to give a mixture of compound IX-Ia and compound I-XXVd. Purification of the crude mixture by column chromatography (PE/EA=3:1) afforded compound IX-Ia (0.5 g, yield 32%). ¹H NMR (400 MHz, CDCl₃): δ 9.74 (s, 1H), 7.65 (d, J=8.0 Hz, 1H), 7.27 (d, J=8.0 Hz, 1H), 7.10 (s, 1H), 4.52 (s, 2H), 2.56 (s, 3H).

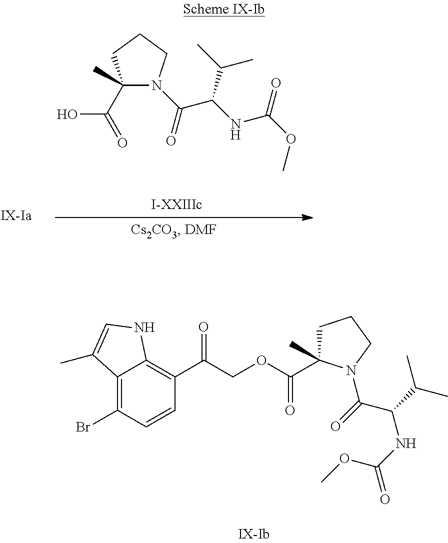

### General Procedure IX-B

Compound IX-Ia (224 mg, 0.68 mmol), compound I-XXIIIc (200 mg, 0.70 mmol) and Cs₂CO₃ (480 mg, 1.5 mmol) and DMF (5 mL) were combined in a flask. The contents of the flask were stirred at room temperature for 2 hours. The mixture was then diluted with EtOAc (30 mL) and the resulting mixture was washed with water and brine, dried over anhydrous Na₂SO₄, concentrated and purified by prep-TLC (PE/EA=1:1) to afford compound IX-Ib (250 mg, yield 69%). MS (ESI) m/z (M+H)⁺ 537.8.

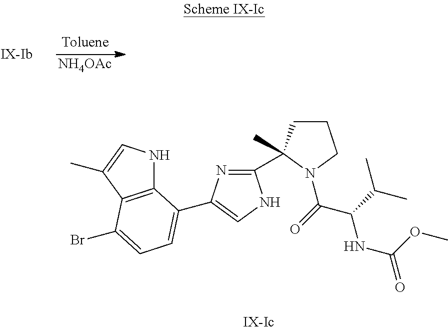

### General Procedure IX-C

In a sealed tube, compound IX-Ib (300 mg, 0.56 mmol) and NH₄OAc (863 mg, 11.2 mmol) in xylene (10 mL) was heated at 180° C. for 5 hours. After cooling to r.t., the mixture was diluted with EtOAc (20 mL), and washed with water and brine. The organic layer was separated, dried over anhydrous Na₂SO₄, and concentrated to afford a crude mixture. The crude mixture was purified by prep-TLC (DCM/MeOH=20:1) to afford compound IX-Ic (50 mg, yield 17%). MS (ESI) m/z (M+H)⁺ 516.

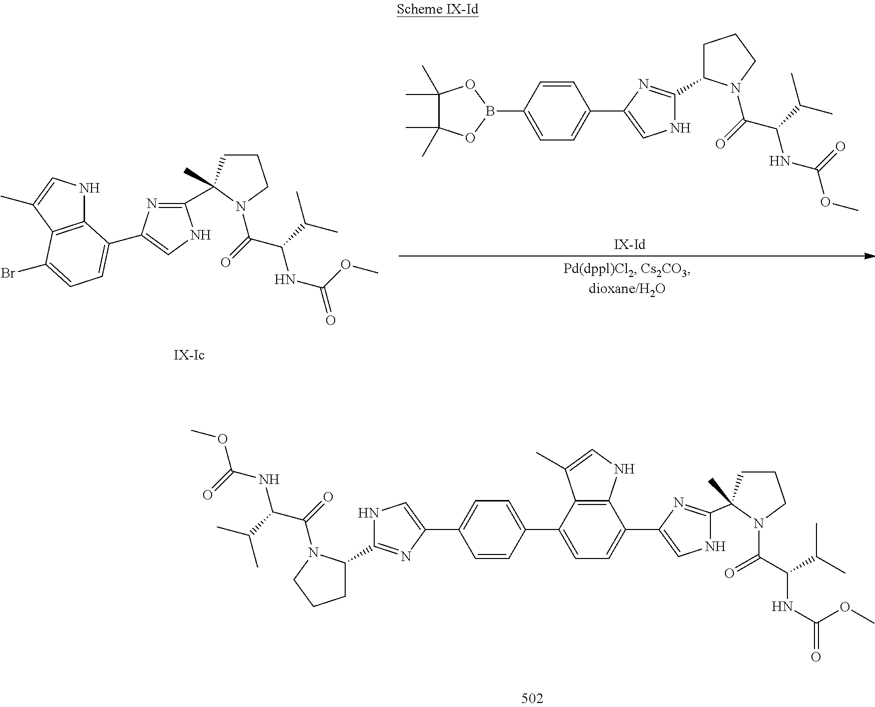

### General Procedure IX-D

To a solution of compound IX-Ic (50 mg, 0.10 mmol) and compound IX-Id (60 mg, 0.12 mmol) in 6 mL of toluene/water (v/v=5/1) was added Pd(dppf)Cl₂ (10% mol) and Cs₂CO₃ (70 mg, 0.20 mmol). The resulting mixture was stirred at 100° C. for 2 hours. After cooling to r.t., the mixture was diluted EtOAc (20 mL) and washed with water and brine. The organic layer was separated, dried over anhydrous Na₂SO₄, and concentrated to provide a residue. The residue was purified by prep-HPLC to afford compound 502 (5 mg, yield 6.4%). ¹H NMR (400 MHz, CD₃OD): δ 7.69-7.80 (m, 2H), 7.34-7.45 (m, 5H), 7.10 (s, 1H), 6.90 (d, J=7.2 Hz, 1H), 5.19-5.22 (m, 1H), 4.22-4.27 (m, 2H), 3.90-4.12 (m, 4H), 3.68 (s, 6H), 2.63-2.68 (m, 1H), 2.03-2.40 (m, 9H), 1.93 (s, 6H), 0.87-1.03 (m, 12H). MS (ESI) m/z (M+H)⁺ 806.4.

### Example IX-IV

### Compound 503 can be Prepared According to the Following Scheme

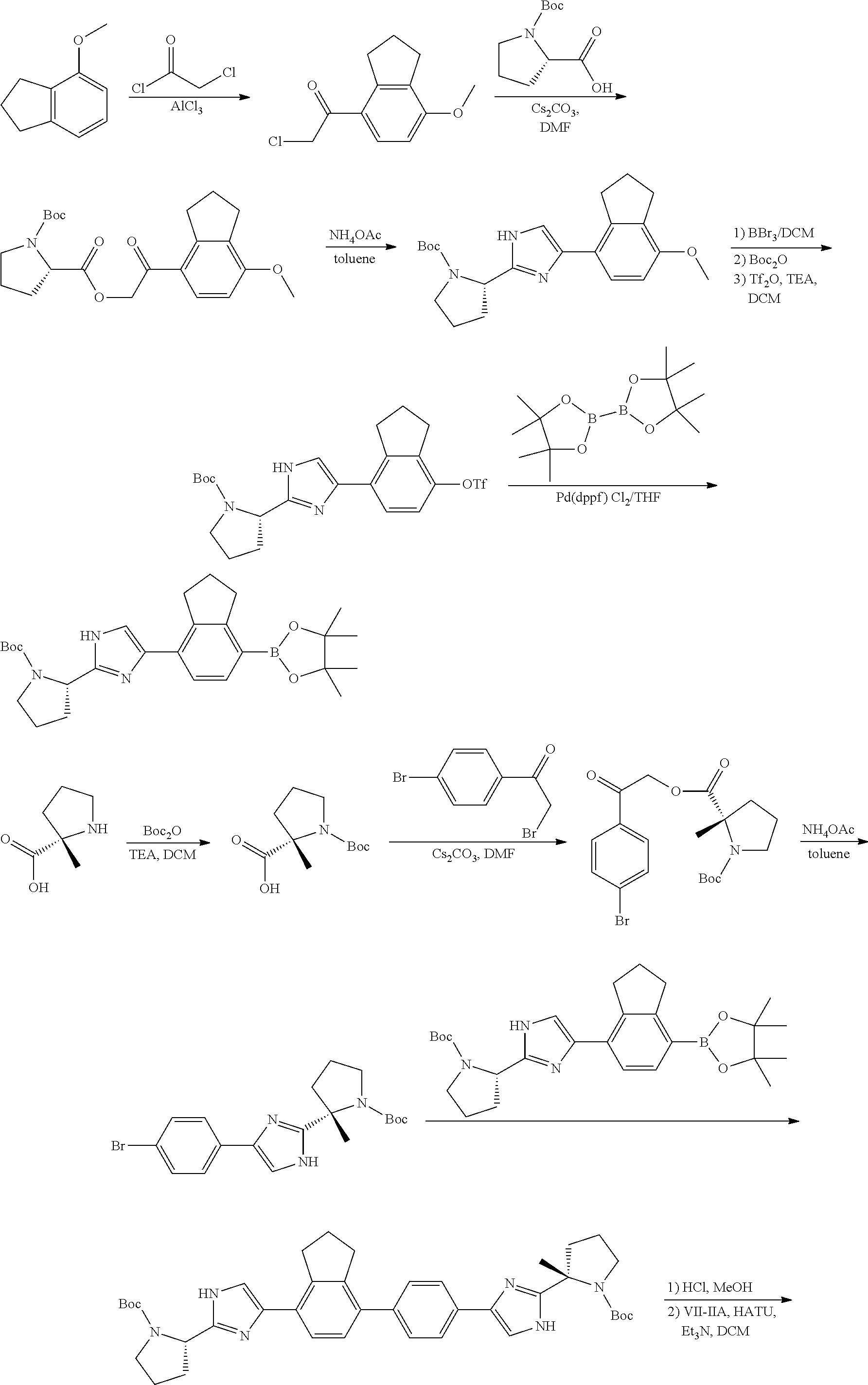

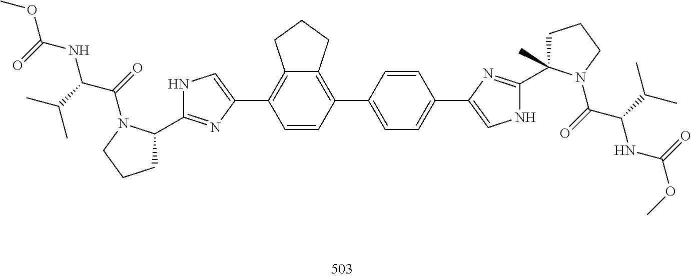

### Example IX-V

### Compound 504 can be Prepared According to the Following Scheme

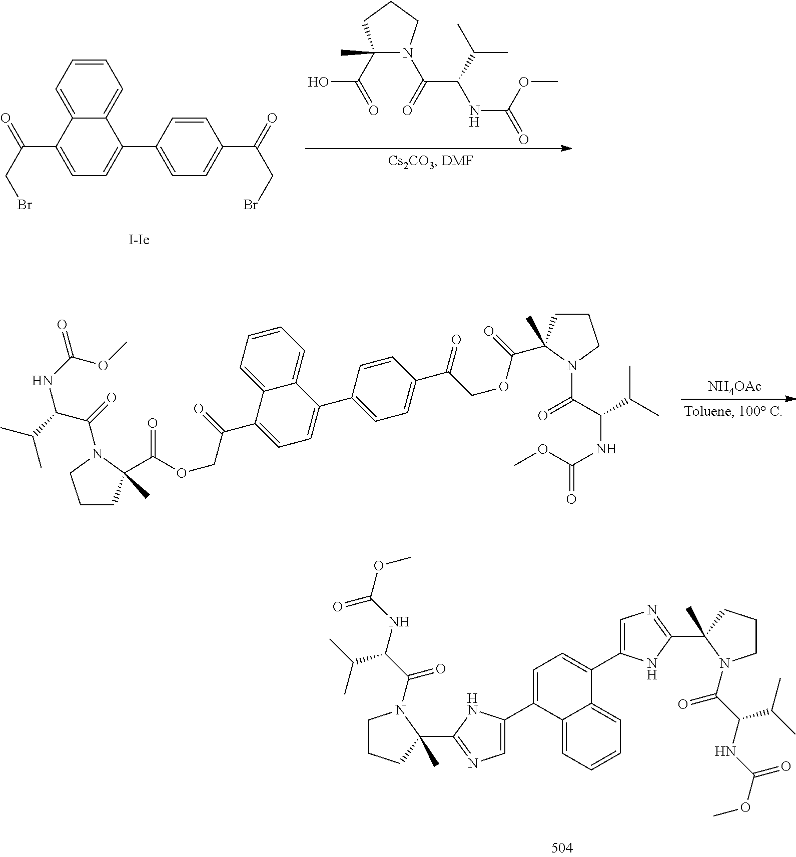

### Example IX-VI

### Compound 505 can be Prepared According to the Following Scheme

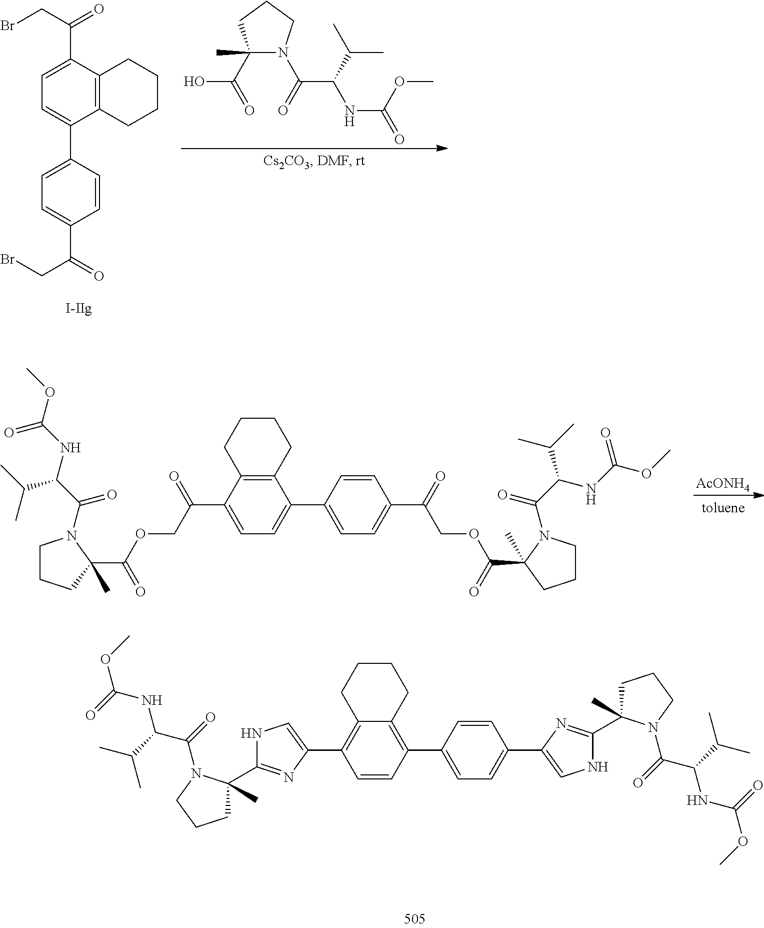

### Example IX-VII

### Compound 506 can be Prepared According to the Following Scheme

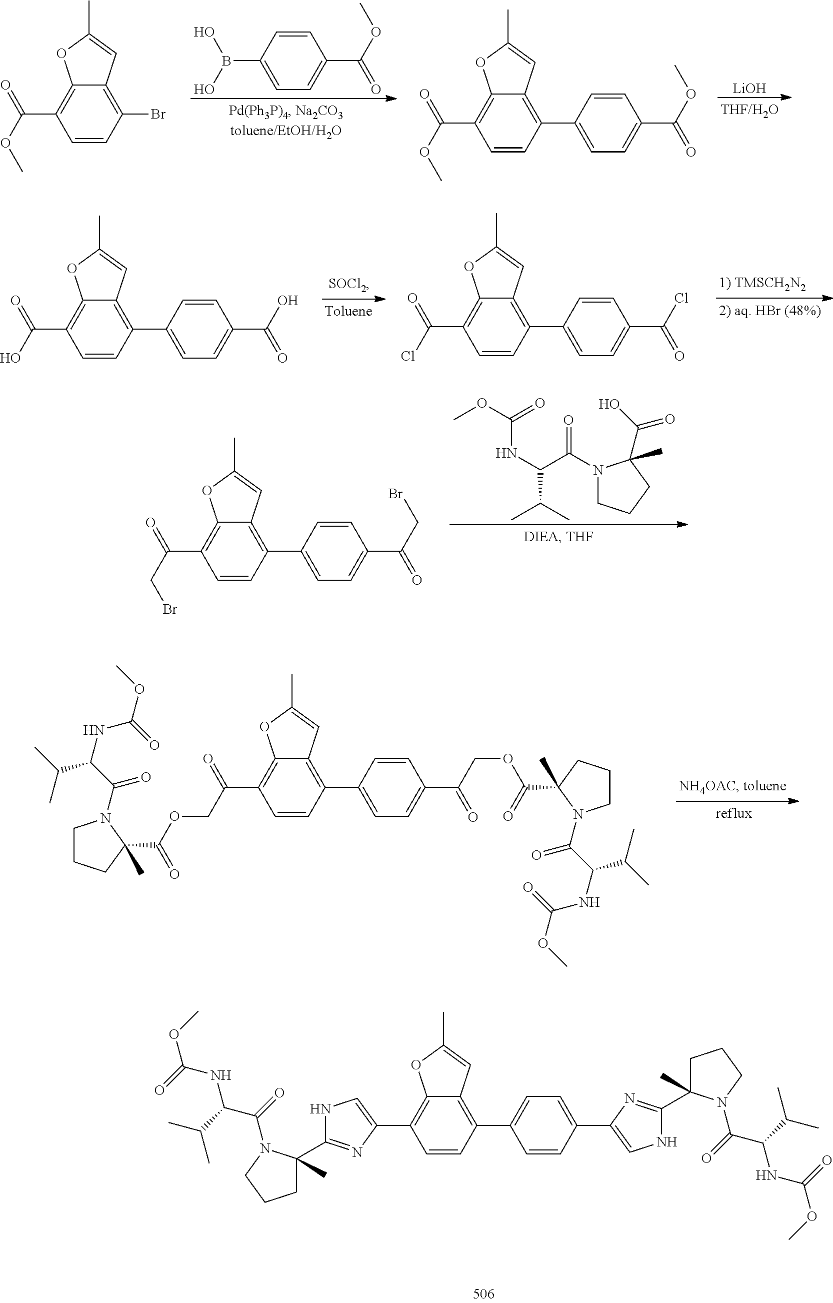

### Example IX-VIII

### Compound 507 can be Prepared According to the Following Scheme

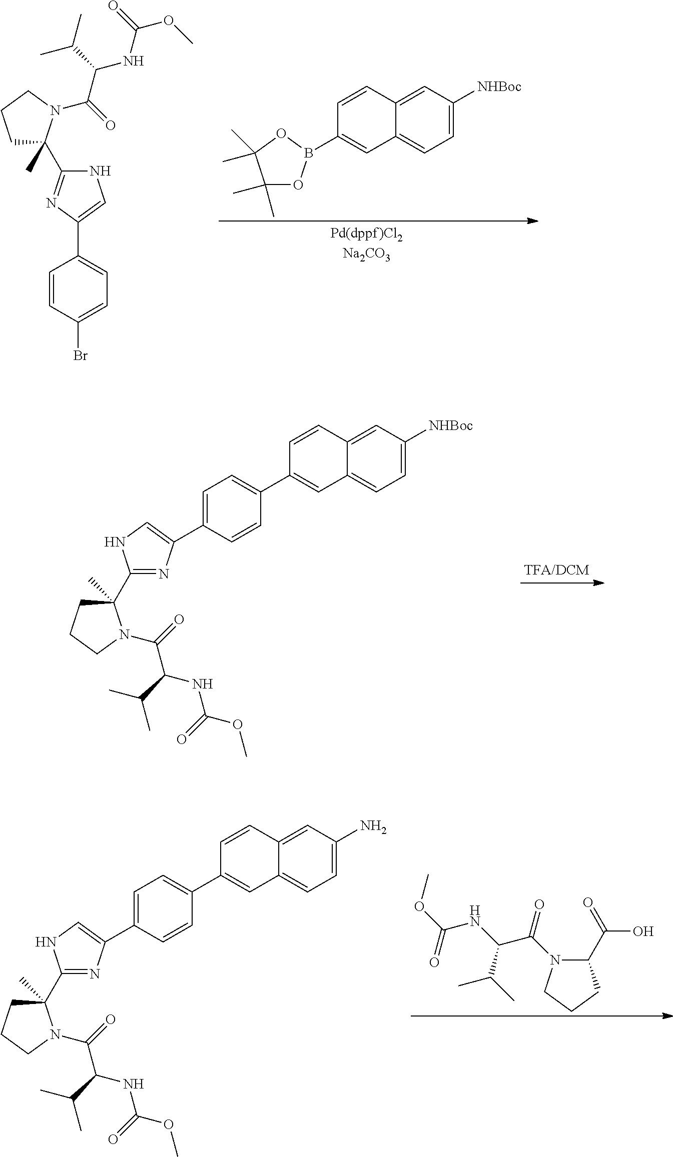

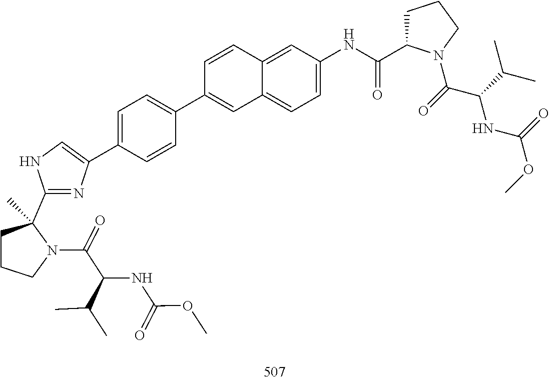

### Example IX-VIV

### Compound 508 can be Prepared According to the Following Scheme

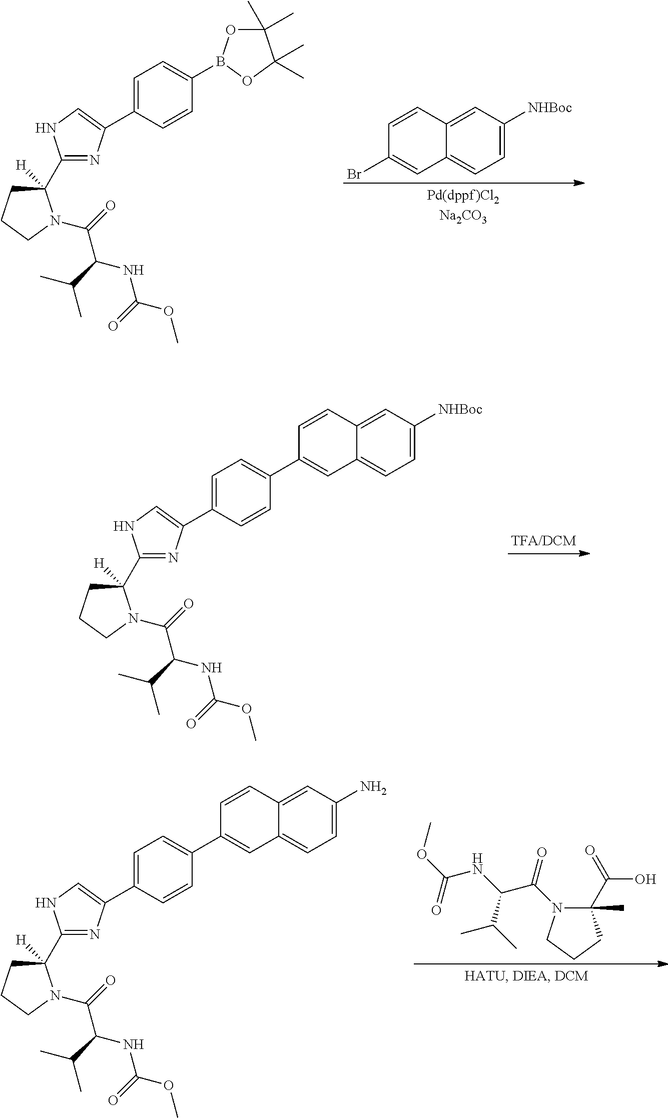

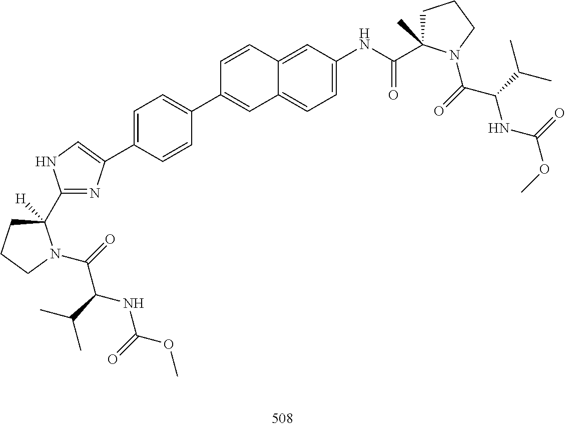

### Section X

HCV Replicon Assay

Huh7 cells containing HCV replicons with an integrated luciferase reporter gene are maintained at 37° C. in 5% CO₂ in Dulbecco's modified Eagle's medium (DMEM; Mediatech, Herndon, Va.) containing 10% heat-inactivated fetal bovine serum (FBS; Mediatech, Herndon, Va.), 2 mM L-glutamine (Cambrex Bioscience, Walkersville, Md.), 1% non essential amino acids (Lonza, Walkersville, Md.), 50 IU/mL penicillin (Mediatech, Herndon, Va.), 50 mg/mL streptomycin (Mediatech, Herndon, Va.) and 0.5 mg/mL G418 (Promega, Madison, Wis.). Cells are sub-divided 1:3 or 4 every 2-3 days.

24 h prior to the assay, Huh7 cells containing sub-genomic HCV replicons are collected, counted, and plated in Nunclon 96-well tissue culture plates (ThermoFisher, Rochester, N.Y.) at 5000 cells/well in 100 mL standard maintenance medium (above) and incubated in the conditions above. To initiate the assay, culture medium is removed, and replaced with 90 mL maintenance media lacking G418. Test compounds are serially diluted three-fold in dimethyl sulfoxide (DMSO) in two duplicate rows for each EC50 determination. These compound solutions are diluted ten-fold in DMEM lacking serum and G418. 10 mL of these compound solutions in media are added to duplicate tissue culture plates. The final volume is 100 μL with a DMSO concentration of 1%. Compound concentrations are adjusted to appropriately define a dose response curve. Typical dilution series range from 100 mM to 1.69 nM final concentrations to 1 nM to 16.9 fM final. Plates are incubated at 37° C. for approximately 48 hr.

Following incubation, media is removed from one of the two duplicate plates and replicon-reporter luciferase activity is measured using a Bright-Glo luciferase assay kit (Promega, Madison, Wis.) according to manufacturer's instructions. Semi-log plots of luciferase activity versus the logarithm of compound concentration are fit to a 4-parameter logistic function using XLfit software (IDBS Inc., Guildford, UK) to determine EC₅₀.

TABLE 20
Examples of activity.

| Compound | EC₅₀ nM |
|---|---|
| 101 | B |
| 102 | B |
| 103 | B |
| 104 | B |
| 201 | C |
| 202 | C |
| 203 | C |
| 204 | B |
| 205 | A |
| 206 | C |
| 207 | C |
| 208 | C |
| 209 | C |
| 210 | A |
| 211 | C |
| 212 | C |
| 214 | B |
| 216 | C |
| 217 | C |
| 221 | C |
| 222 | C |
| 301 | C |
| 302 | C |
| 303 | B |
| 304 | C |
| 305 | A |
| 306 | C |
| 307 | C |
| 308 | C |
| 309 | C |
| 310 | C |
| 311 | C |
| 312 | C |
| 314 | C |
| 315 | C |
| 323 | C |
| 324 | C |
| 325 | C |
| 326 | A |
| 327 | C |
| 328 | C |
| 329 | C |
| 330 | C |
| 401 | C |
| 402 | C |
| 403 | C |
| 418 | C |
| 419 | C |
| 420 | C |

A indicates an EC₅₀ of greater than 100 nM
B indicates an EC₅₀ between 10 and 100 nM
C indicates an EC₅₀ of less than 10 nM

What is claimed is:

1. A compound having the structure of Formula I: 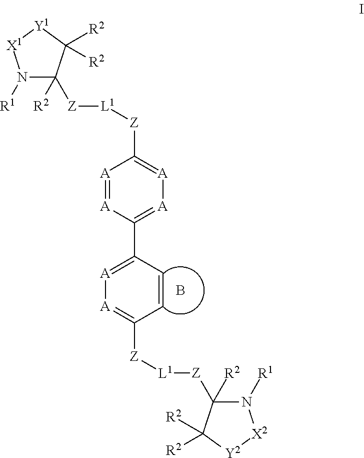
or a pharmaceutically acceptable salt thereof,
wherein:
each R¹ is separately selected from the group consisting of hydrogen, R^{1a}S(O₂)—, R^{1a}C(═O)— and R^{1a}C(═S)—;
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;
each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;
each R^{e}R^{f}N is separately selected, wherein R^{e} and R^{f} are each separately selected from the group consisting of hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;
each R^{x}R^{y}N is separately selected, wherein R^{x} and R^{y} are each separately selected from the group consisting of hydrogen, alkylOC(═O)—, C₁₋₆alkyl, C₁₋₆alkylC(═O)—, aryl, arylalkyl, cycloalkyl, and heterocyclyl;
each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is 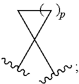
each R^{3a} is separately selected from the group consisting of hydrogen, and optionally substituted C₁₋₆alkyl;
each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;
each R^{4a}R^{4b}N is separately selected, wherein R^{4a} and R^{4b} are each separately selected from the group consisting of hydrogen, optionally substituted C₁₋₆alkyl, and aryl(CH₂)ₙ—;
each R^{5a} is separately selected from the group consisting of optionally substituted C₁₋₆alkyl, and aryl(CH₂)ₙ—;
each R^{6a} is separately selected from the group consisting of optionally substituted C₁₋₆alkyl, and aryl(CH₂)ₙ—;
X¹ is (C(R²)₂)_{q}, 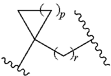
or X₁ is null;
Y¹ is selected from O (oxygen), S (sulfur), S(O), SO₂, NR², and C(R²)₂ with the proviso that when X₁ is null Y₁ is C(R²)₂;
X² is (C(R²)₂)_{q}, 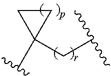
or X² is null;
Y² is selected from O (oxygen), S (sulfur), S(O), SO₂, NR², and C(R²)₂ with the proviso that when X² is null Y² is C(R²)₂;
each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups;
each Z is separately selected, wherein Z is selected from the group consisting of O (oxygen) and CH₂, or Z is null;
each A is separately selected from the group consisting of CR³ and N (nitrogen);
each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 9 halo and up to 5 hydroxy;
each L₁ is separately selected from the group consisting of 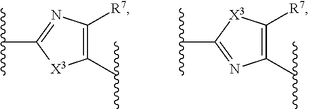
—(═O)(CH₂)ₘOC(═O)—, —C(CF₃)₂NR^{2c}—, and 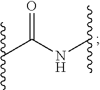
each X³ is separately selected from the group consisting of NH, NC₁₋₆alkyl, O (oxygen), and S (sulfur);
each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo;
each R^{a}R^{b}N is separately selected, wherein R^{a} and R^{b} are each separately selected from the group consisting of hydrogen, C₂₋₆alkenyl, and C₁₋₆alkyl;
each m separately is 1 or 2;
each n separately is 0, 1 or 2;
each p separately is 1, 2, 3 or 4;
each q separately is 1, 2, 3, 4 or 5;
each r separately is 0, 1, 2, 3, or 4;
B is a fused optionally substituted saturated or unsaturated three- to seven-membered carbocyclic ring, a fused optionally substituted saturated or unsaturated three- to seven-membered heterocyclic ring, or a fused optionally substituted five- or six-membered heteroaryl ring, each optionally substituted with one or more R⁴; and
each R⁴ is separately selected from the group consisting of C₁₋₆alkoxy, C₁₋₆alkylOC₁₋₆alkyl, C₁₋₆ alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, C₁₋₆alkyl optionally substituted with up to 9 halo and up to 5 hydroxy, or optionally two geminal R⁴ are together oxo;
provided that the compound does not have the following structure:

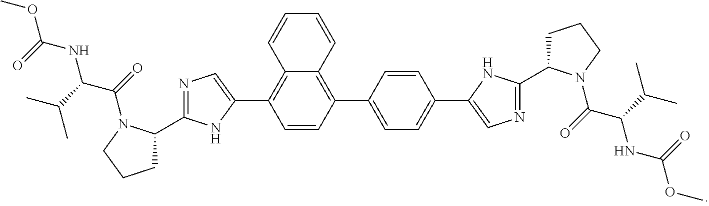

2. The compound of claim 1,
wherein:
each R¹ is separately selected from the group consisting of hydrogen and
R^{1a}C(═O)— and R^{1a}C(═S)—;
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, (R^{c}R^{d}N)(CH═CH)ₘ—, (R^{c}R^{d}N)alkyl, (R^{c}R^{d}N)C(═O)—, C₁₋₆alkoxy optionally substituted with up to 5 halo, and C₁₋₆alkyl optionally substituted with up to 5 halo;
each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 5 halo, and C₁₋₆alkyl optionally substituted with up to 5 halo;
each R^{2a} is separately selected from the group consisting of hydrogen, C₁₋₆alkyl, aryl(CH₂)ₙ—, and heteroaryl(CH₂)ₙ—;
each R^{3a} is separately selected from the group consisting of hydrogen, and C₁₋₆alkyl;
each R^{3b} is separately selected from the group consisting of C₁₋₆alkyl, —(CH₂)ₙC(═O)NR^{4a}R^{4b}, —(CH₂)ₙC(═O)OR^{5a}, and —(CH₂)ₙC(═O)R^{6a};
each R^{4a}R^{4b}N is separately selected, wherein R^{4a} and R^{4b} are each separately selected from the group consisting of hydrogen, C₁₋₆alkyl, and aryl(CH₂)ₙ—;
each R^{5a} is separately selected from the group consisting of C₁₋₆alkyl, and aryl(CH₂)ₙ—;
each R^{6a} is separately selected from the group consisting of C₁₋₆alkyl, and aryl(CH₂)ₙ—;
X¹ is C(R²)₂, or X₁ is null;
Y¹ is selected from O (oxygen), S (sulfur), S(O), SO₂, and C(R²)₂ with the proviso that when X₁ is null Y₁ is C(R²)₂;
X² is C(R²)₂, or X² is null;
Y² is selected from O (oxygen), S (sulfur), S(O), SO₂, and C(R²)₂ with the proviso that when X² is null Y² is C(R²)₂;
each X³ is separately selected from the group consisting of NH, O (oxygen), and S (sulfur);
each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 5 halo, or optionally two vicinal R² and the carbons to which they are attached are together a fused three- to six-membered carbocyclic ring optionally substituted with up to two C₁₋₆alkyl groups;
each L₁ is separately selected from the group consisting of 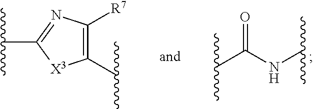
each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 5 halo and up to 5 hydroxy;
each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 5 halo; and
each R⁴ is separately selected from the group consisting of C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, C₁₋₆alkyl optionally substituted with up to 5 halo and up to 5 hydroxy, or optionally two geminal R⁴ are together oxo.

3. The compound of claim 1,
wherein: 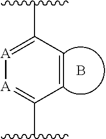
is selected from the group consisting of: 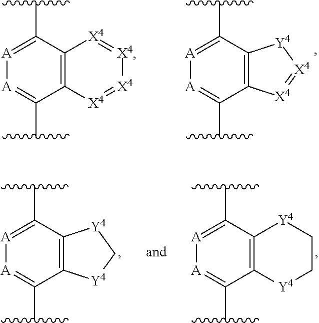
wherein,
each X⁴ is separately selected from the group consisting of CH, CR⁴ and N (nitrogen); and
each Y⁴ is separately selected from the group consisting of CH₂, CHR⁴, C(R⁴)₂, NR⁴, O (oxygen), and S (sulfur).

4. The compound of claim 1, wherein each Z is null.

5. The compound of claim 1 having the structure of Formula Ia: 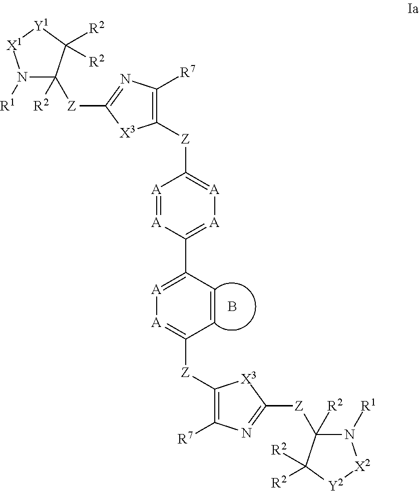
or a pharmaceutically acceptable salt thereof.

6. The compound of claim 5 having the structure of Formula Ib: 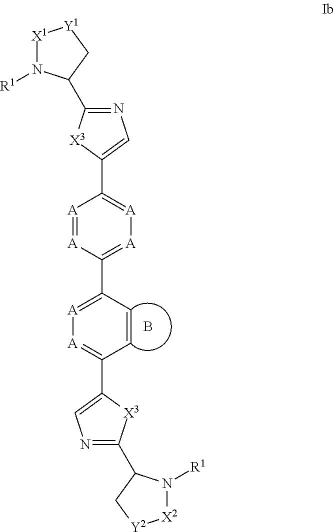
or a pharmaceutically acceptable salt thereof.

7. The compound of claim 6, wherein each R¹ is R^{1a}C(═O)—.

8. The compound of claim 7, wherein each R^{1a} is —CHR^{2a}NHR^{3b}.

9. The compound of claim 8, wherein each R^{2a} is C₁₋₆alkyl;
each R^{3b} is —C(═O)OR⁵; and
each R⁵ is C₁₋₆alkyl.

10. The compound of claim 1, having the structure: 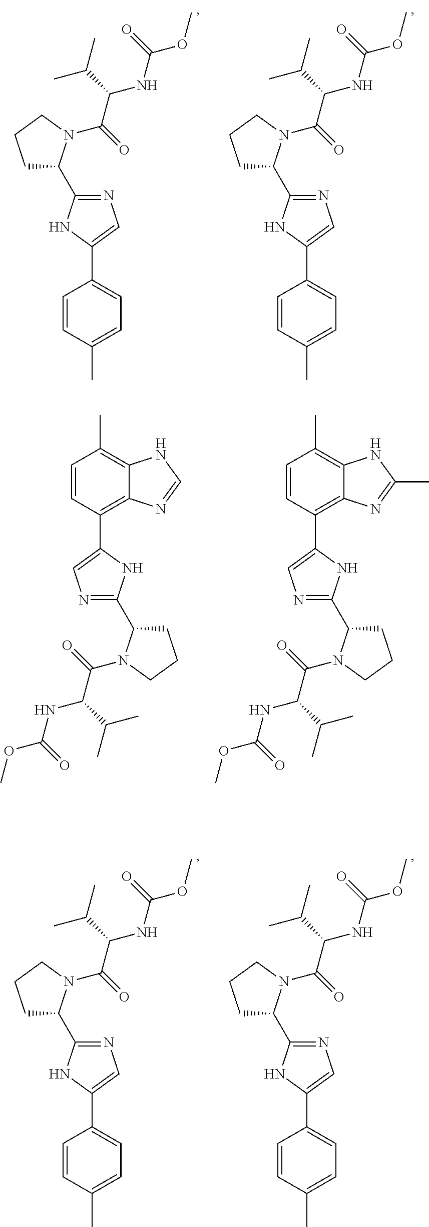 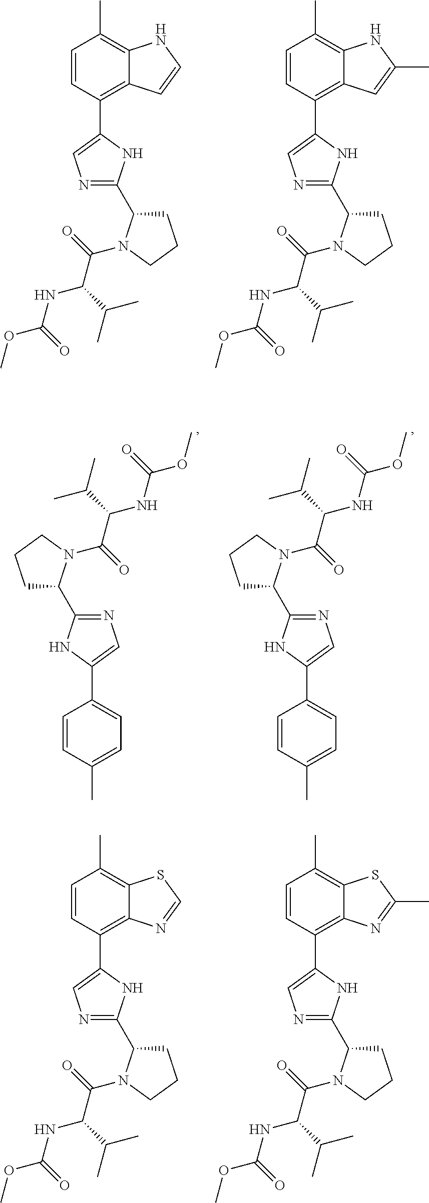 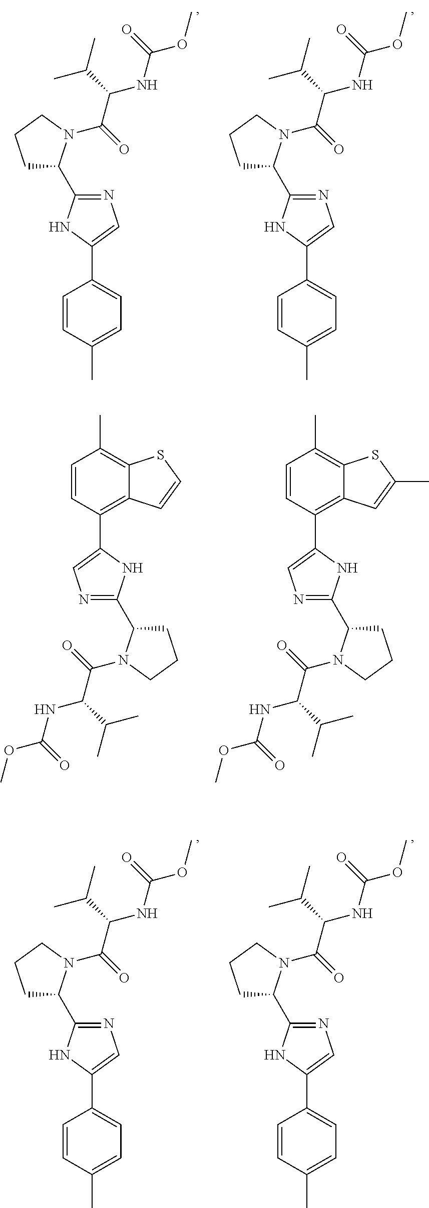 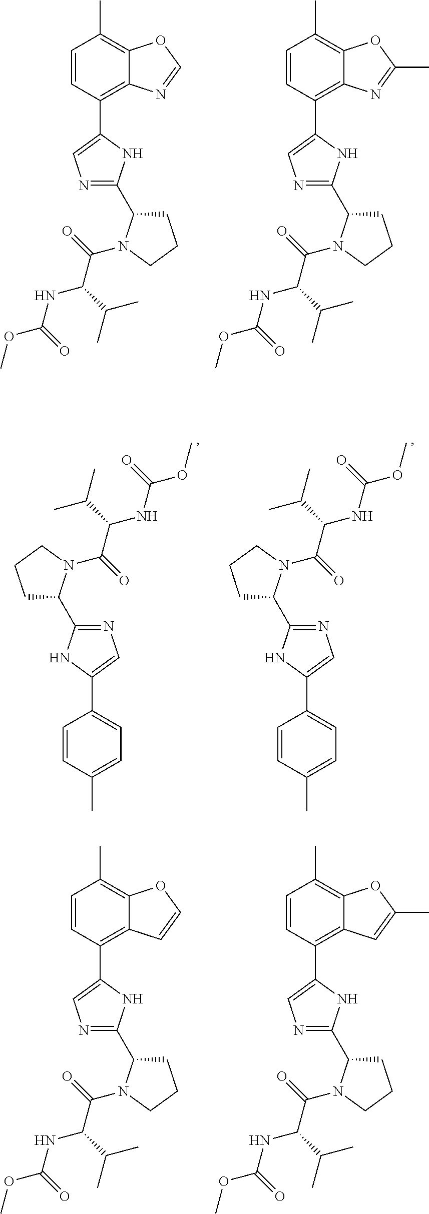 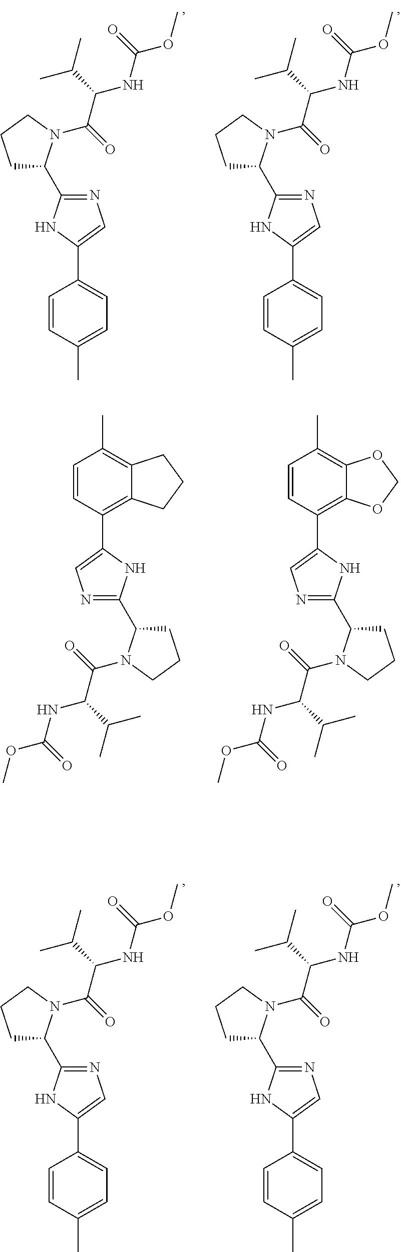 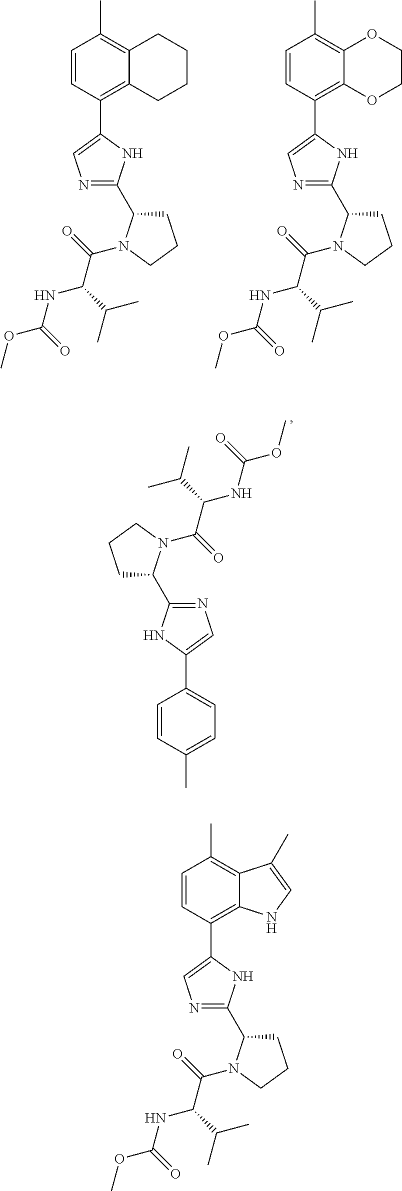 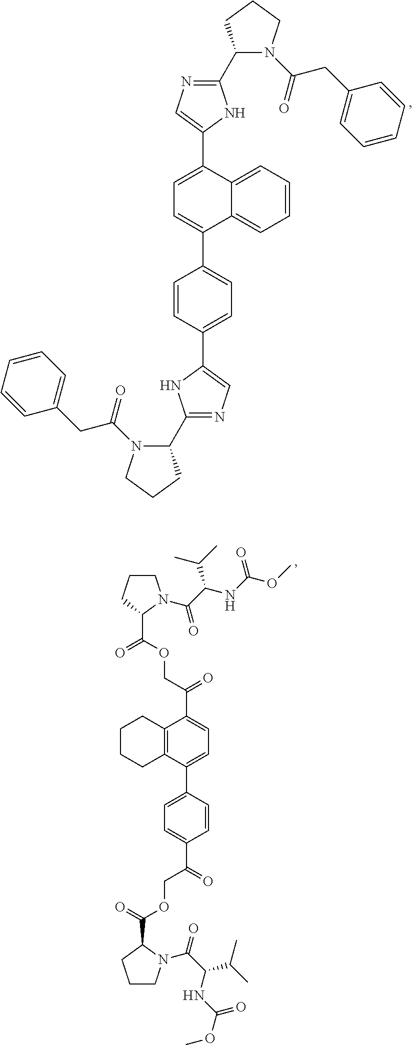 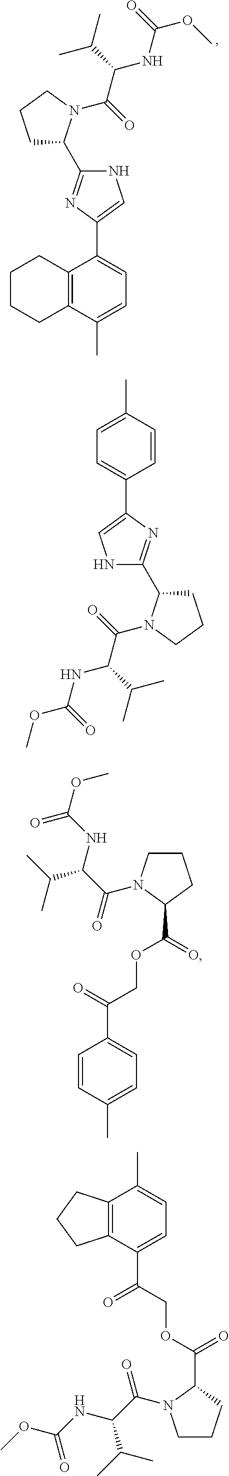 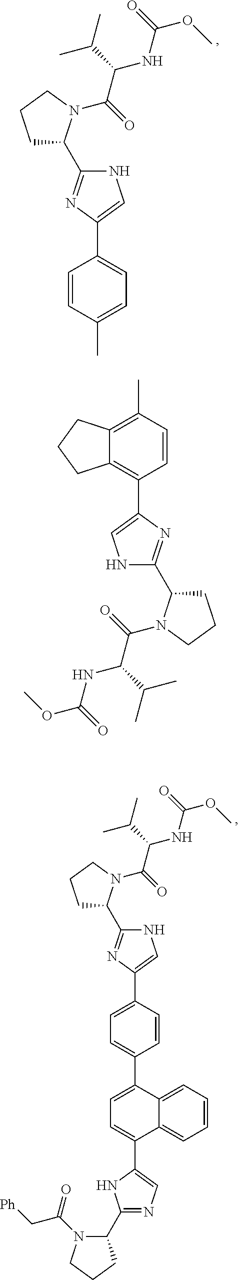 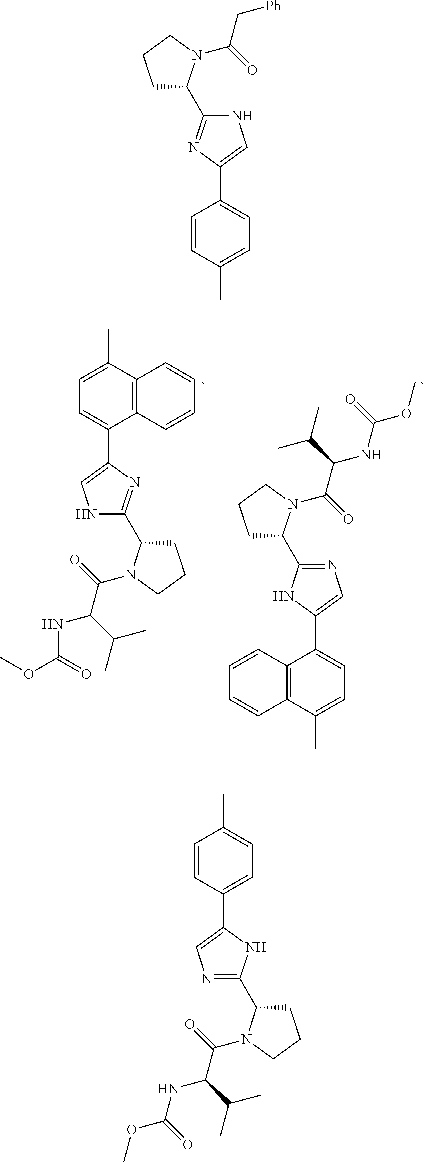 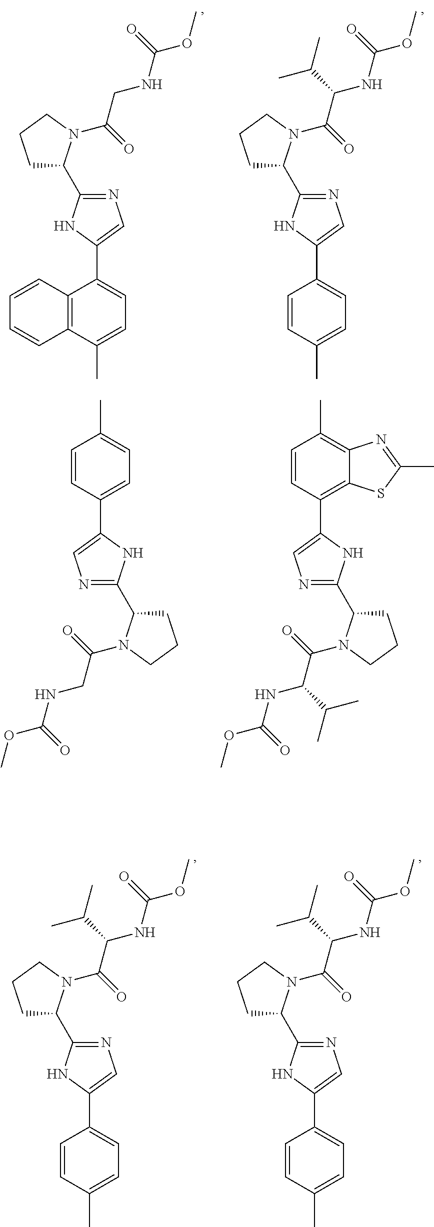 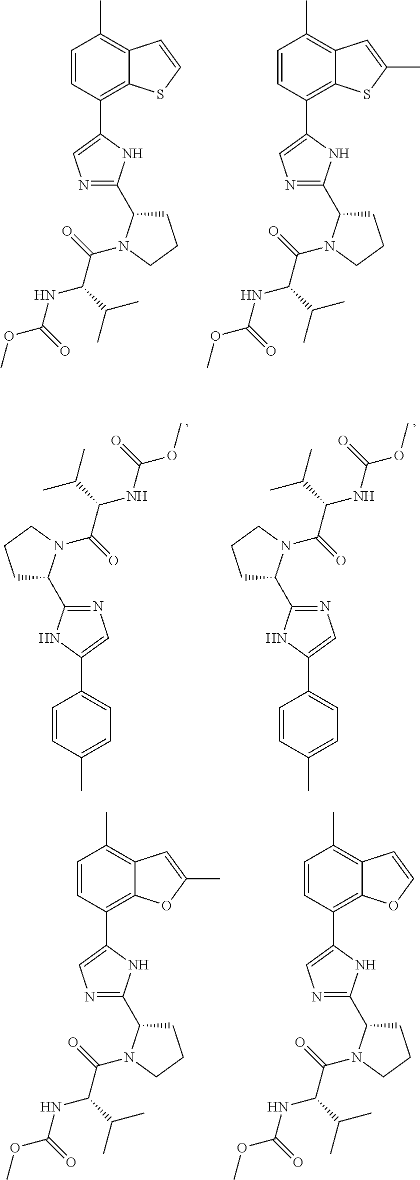 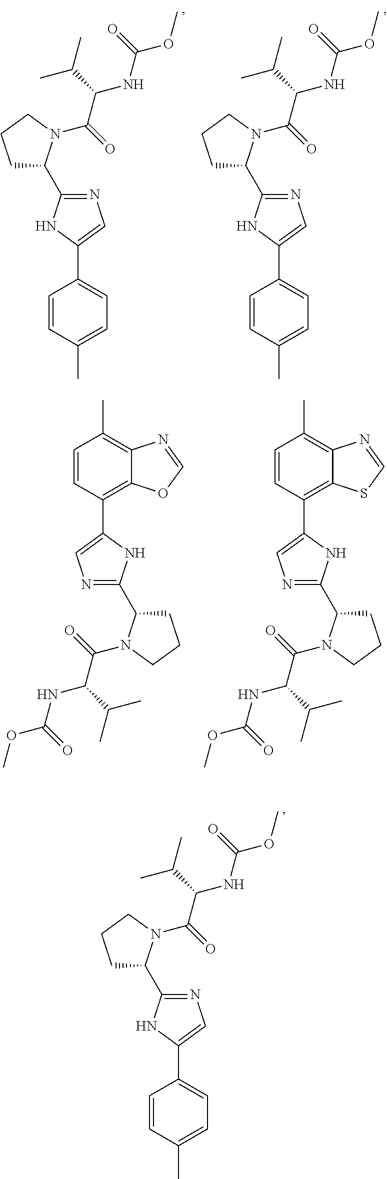 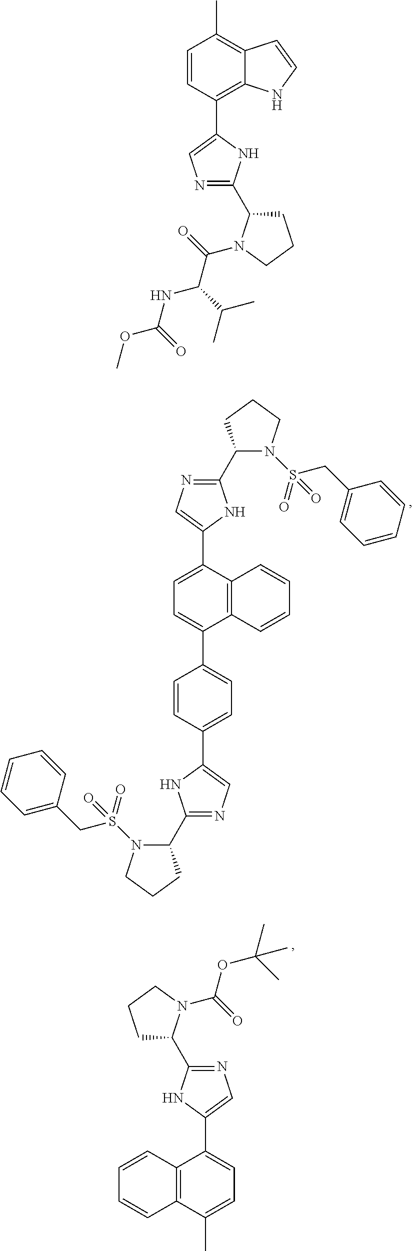 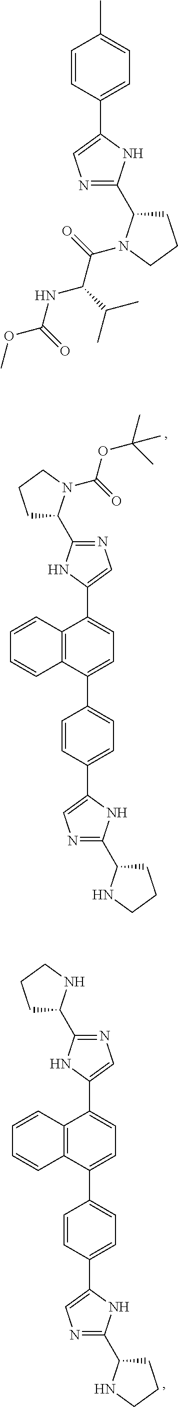 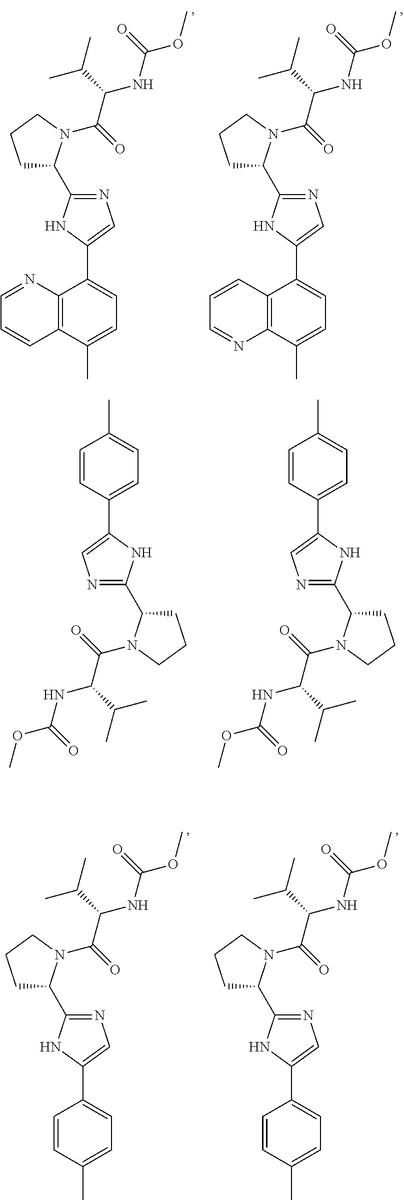 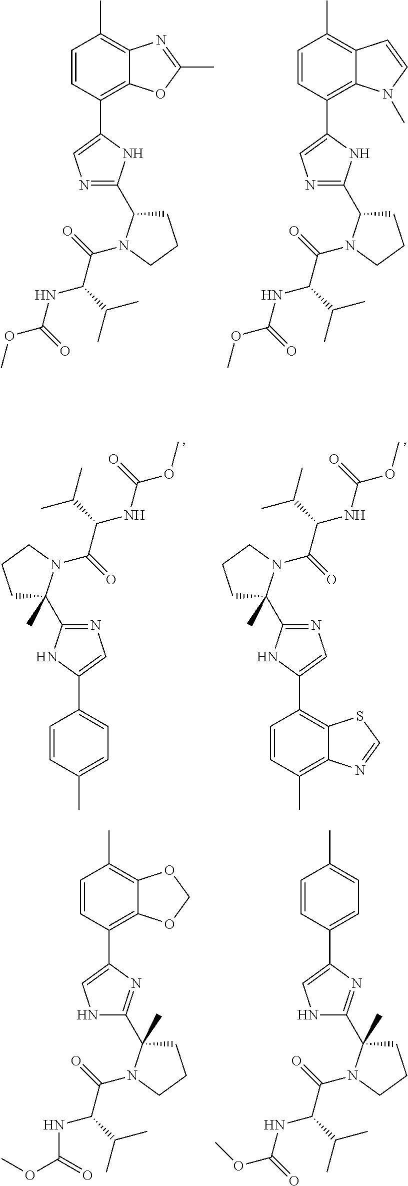 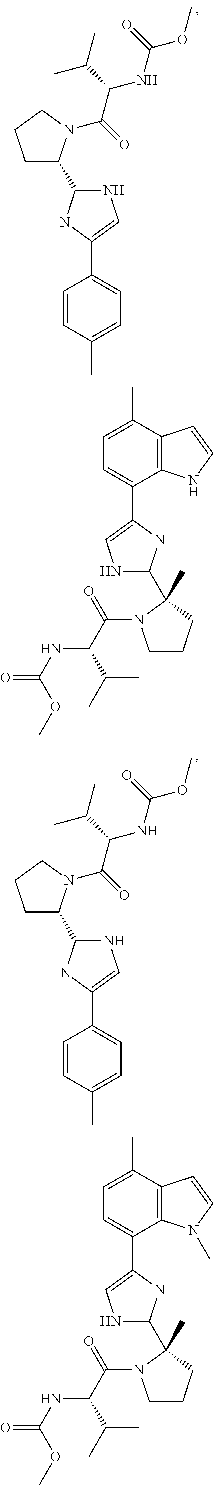 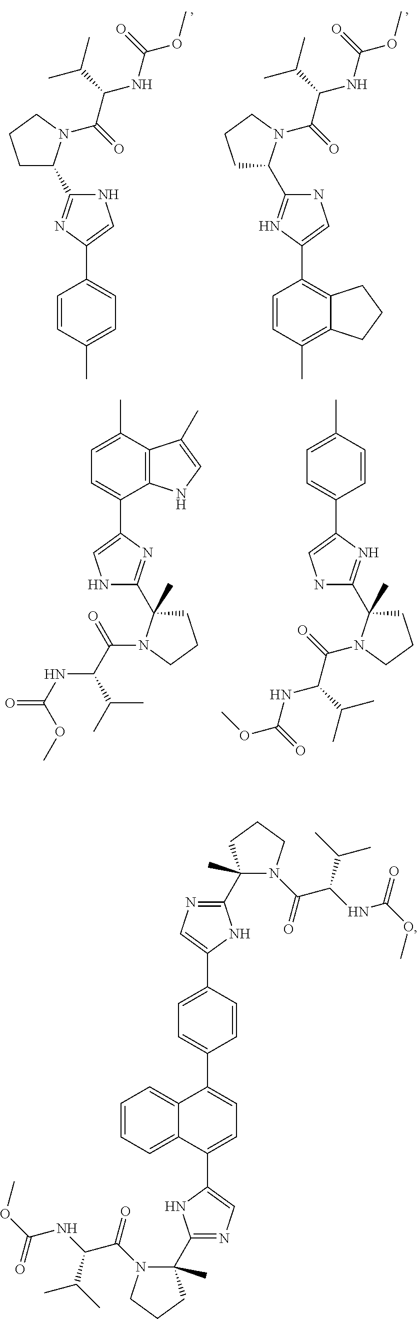 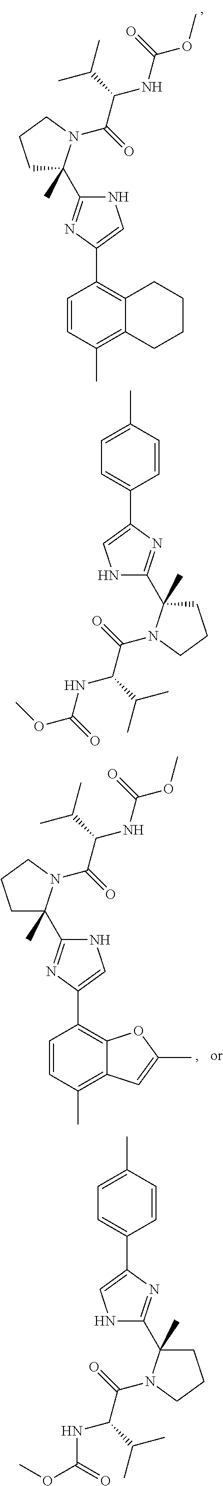 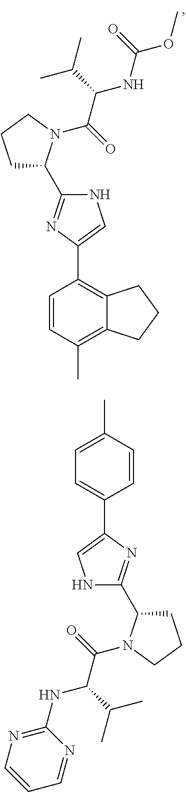
or a pharmaceutically acceptable salt thereof.

11. The compound of claim 1 having the structure of Formula Ic: 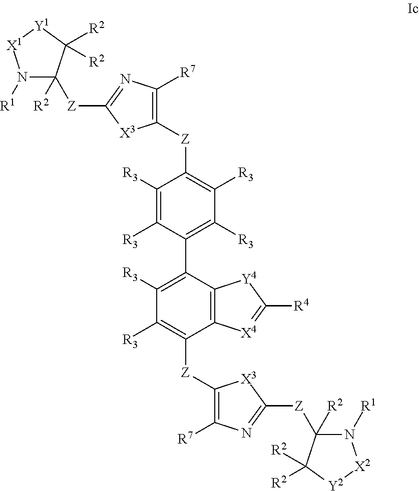
or a pharmaceutically acceptable salt thereof, wherein:
each X⁴ is separately selected from the group consisting of CH, CR⁴ and N (nitrogen); and
each Y⁴ is separately selected from the group consisting of CH₂, CHR⁴, C(R⁴)₂, NR⁴, O (oxygen), and S (sulfur).

12. The compound of claim 1 having the structure of Formula Id: 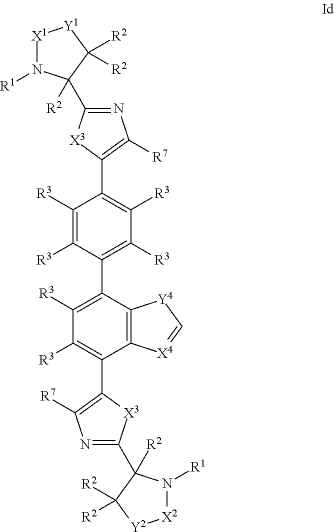
or a pharmaceutically acceptable salt thereof, wherein:
each X⁴ is separately selected from the group consisting of CH, CR⁴ and N (nitrogen); and
each Y⁴ is separately selected from the group consisting of CH₂, CHR⁴, C(R⁴)₂, NR⁴, O (oxygen), and S (sulfur).

13. The compound of claim 1 having the structure of Formula Ie: 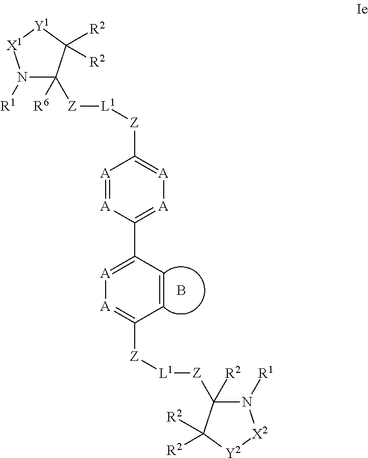
or a pharmaceutically acceptable salt thereof, wherein:
R⁶ is C₁₋₆alkyl optionally substituted with up to 9 halo.

14. The compound of claim 1 having the structure of Formula If: 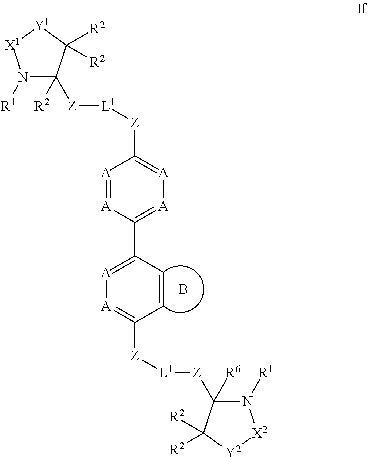
or a pharmaceutically acceptable salt thereof, wherein:
R⁶ is C₁₋₆alkyl optionally substituted with up to 9 halo.

15. A compound having the structure of Formula II: 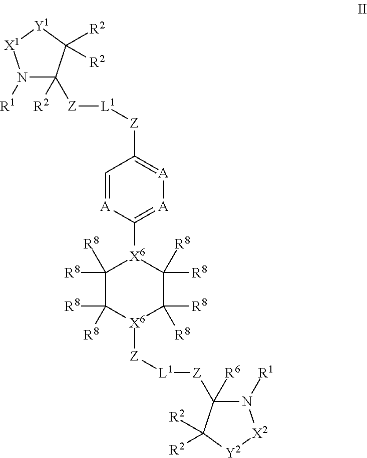
or a pharmaceutically acceptable salt thereof,
wherein:
each R¹ is separately selected from the group consisting of hydrogen and R^{1a}C(═O)— and R^{1a}C(═S)—;
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;
each R^{c}R^{d}N is separately selected, wherein R^{c} and R^{d} are each independently selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;
each R^{e}R^{f}N is separately selected, wherein R^{e} and R^{f} are each separately selected from the group consisting of hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O);
each R^{x}R^{y}N is separately selected, wherein R^{x} and R^{y} are each separately selected from the group consisting of hydrogen, alkylOC(═O)—, alkyl, alkylC(═O)—, aryl, arylalkyl, cycloalkyl, and heterocyclyl;
each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is 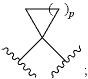
each R^{3a} is separately selected from the group consisting of hydrogen, and optionally substituted C₁₋₆alkyl;
each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;
each R^{4a}R^{4b}N is separately selected, wherein R^{4a} and R^{4b} are each separately selected from the group consisting of hydrogen, optionally substituted C₁₋₆alkyl, and aryl(CH₂)ₙ—;
each R^{5a} is separately selected from the group consisting of optionally substituted C₁₋₆alkyl, and aryl(CH₂)ₙ—;
each R^{6a} is separately selected from the group consisting of optionally substituted C₁₋₆alkyl, and aryl(CH₂)ₙ—;
X¹ is (C(R²)₂)_{q}, 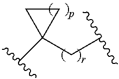
or X¹ is null;
Y¹ is selected from O (oxygen), S (sulfur), S(O), SO₂, NR², and C(R²)₂ with the proviso that when X¹ is null Y¹ is C(R²)₂;
X² is (C(R²)₂)_{q}, 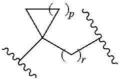
or X² is null;
Y² is selected from O (oxygen), S (sulfur), S(O), SO₂, NR², and C(R²)₂ with the proviso that when X² is null Y² is C(R²)₂;
each X⁶ is separately selected from the group consisting of N (nitrogen), and CR⁸;
each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl group;
each R^{a}R^{b}N is separately selected, wherein R^{a} and R^{b} are each separately selected from the group consisting of hydrogen, C₂₋₆alkenyl, and C₁₋₆alkyl;
each Z is separately selected, wherein Z is selected from the group consisting of O (oxygen) and CH₂, or Z is null;
each A is separately selected from the group consisting of CR³ and N (nitrogen);
each L¹ is separately selected from the group consisting of 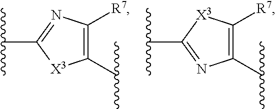
—C(═O)(CH₂)ₘOC(═O)—, —C(CF₃)₂NR^{2c}—, and 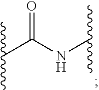
each X³ is separately selected from the group consisting of NH, NC₁₋₆alkyl, O (oxygen), and S (sulfur);
each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 9 halo and up to 5 hydroxy;
each m separately is 1 or 2;
each n separately is 0, 1 or 2;
each p separately is 1, 2, 3 or 4;
each q separately is 1, 2, 3, 4 or 5;
each r separately is 0, 1, 2, 3, or 4;
each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo; and
each R⁸ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 9 halo and up to 5 hydroxy, or optionally two geminal R⁸ are together oxo;
wherein at least one A is N (nitrogen) or both X⁶ are N (nitrogen);
provided that the compound is not selected from the group consisting of:

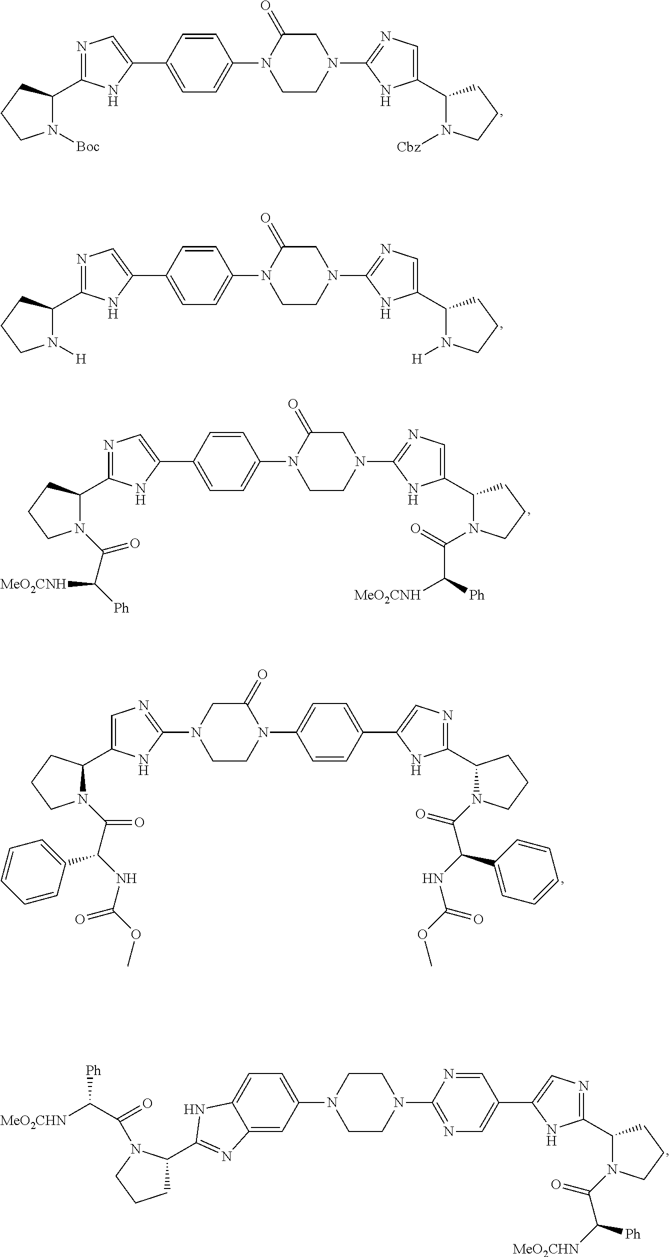

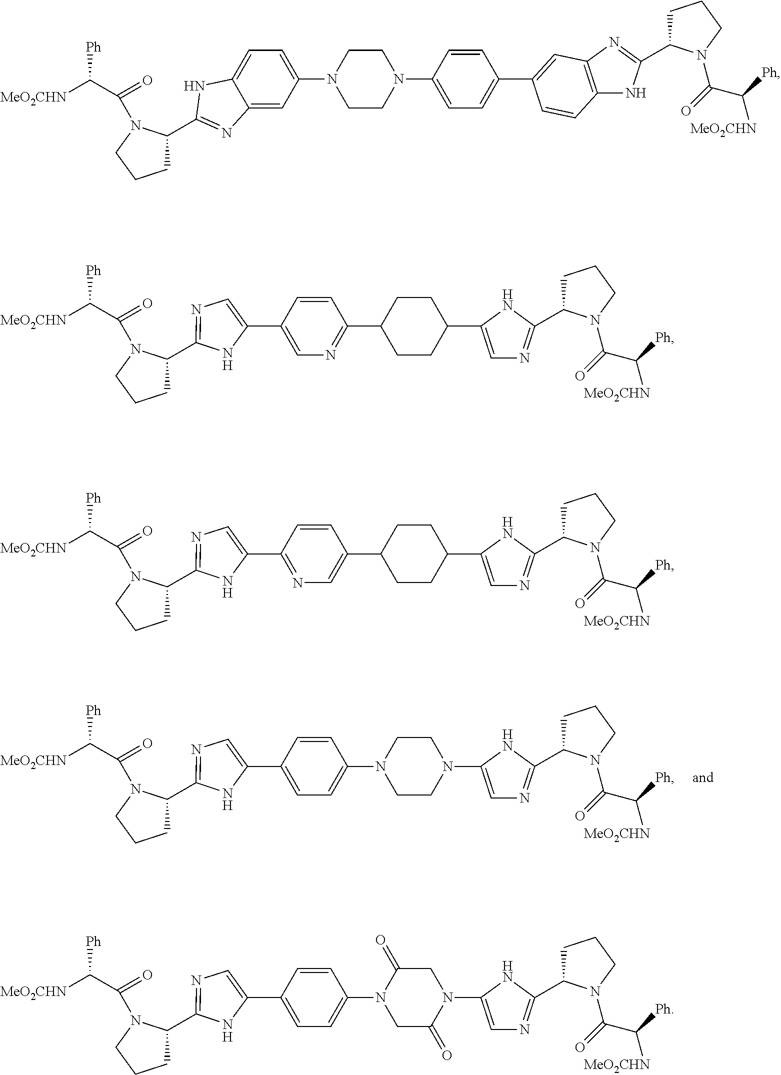

16. The compound of claim 15,
wherein:
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, (R^{c}R^{d}N)(CH═CH)ₘ—, (R^{c}R^{d}N)alkyl, (R^{c}R^{d}N)C(═O)—, C₁₋₆alkoxy optionally substituted with up to 5 halo, and C₁₋₆alkyl optionally substituted with up to 5 halo;
each R^{c}R^{d}N is separately selected, wherein R^{c} and R^{d} are each independently selected from the group consisting of hydrogen, alkoxyC(═O)—, C₁₋₆alkyl, C₁₋₆alkylC(═O)—, C₁₋₆alkylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 5 halo, and C₁₋₆alkyl optionally substituted with up to 5 halo;
each R^{2a} is separately selected from the group consisting of hydrogen, C₁₋₆alkyl, aryl(CH₂)ₙ—, and heteroaryl(CH₂)ₙ—;
each R^{3a} is separately selected from the group consisting of hydrogen, and C₁₋₆alkyl;
each R^{3b} is separately selected from the group consisting of C₁₋₆alkyl, —(CH₂)ₙC(═O)NR^{4a}R^{4b}, —(CH₂)ₙC(═O)OR^{5a}, and —(CH₂)ₙC(═O)R^{6a};
each R^{4a}R^{4b}N is separately selected, wherein R^{4a} and R^{4b} are each separately selected from the group consisting of hydrogen, C₁₋₆alkyl, and aryl(CH₂)ₙ—;
each R^{5a} is separately selected from the group consisting of C₁₋₆alkyl, and aryl(CH₂)ₙ—;
each R^{6a} is separately selected from the group consisting of C₁₋₆alkyl, and aryl(CH₂)ₙ—;
X¹ is C(R²)₂, or X¹ is null;
Y¹ is selected from O (oxygen), S (sulfur), S(O), SO₂, and C(R²)₂ with the proviso that when X¹ is null Y¹ is C(R²)₂;
X² is C(R²)₂, or X² is null;
Y² is selected from O (oxygen), S (sulfur), S(O), SO₂, and C(R²)₂ with the proviso that when X² is null Y² is C(R²)₂;
each X³ is separately selected from the group consisting of NH, O (oxygen), and S (sulfur);
each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 5 halo, or optionally two vicinal R² and the carbons to which they are attached are together a fused three- to six-membered carbocyclic ring optionally substituted with up to two C₁₋₆alkyl group;
each L¹ is separately selected from the group consisting of 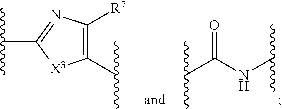
each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 5 halo and up to 5 hydroxy;
each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 5 halo; and
each R⁸ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 5 halo and up to 5 hydroxy, or optionally two geminal R⁸ are together oxo.

17. The compound of claim 15 having the structure of Formula IIa: 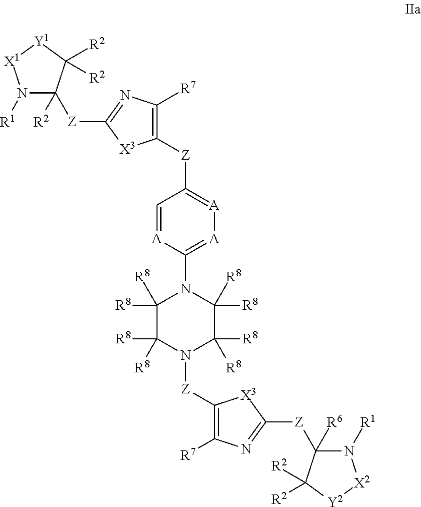
or a pharmaceutically acceptable salt thereof.

18. The compound of claim 17, wherein each Z is null.

19. The compound of claim 18 having the structure of Formula IIb: 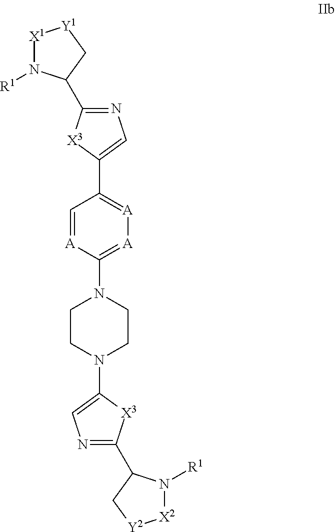
or a pharmaceutically acceptable salt thereof.

20. The compound of claim 19, wherein each R¹ is R^{1a}C(═O)—.

21. The compound of claim 20, wherein each R^{1a} is —CHR^{2a}NHR^{3b}.

22. The compound of claim 21, wherein each R^{2a} is C₁₋₆alkyl;
each R^{3b} is —C(═O)OR⁵; and
each R⁵ is C₁₋₆alkyl.

23. The compound of claim 15, having the structure: 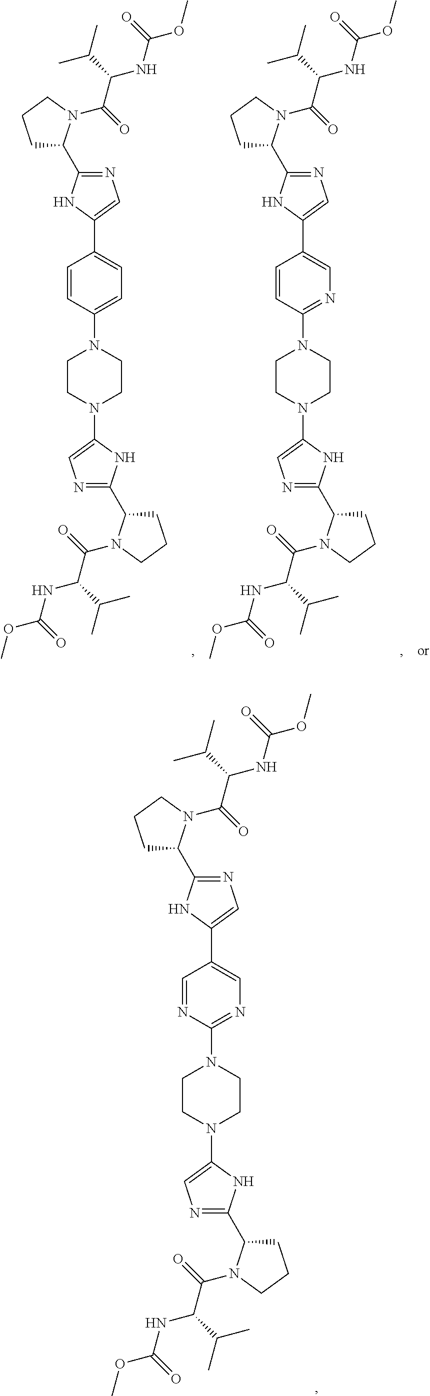
or a pharmaceutically acceptable salt thereof.

24. A compound having the structure of Formula III: 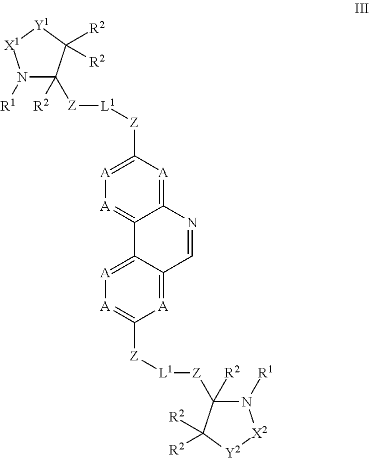
or a pharmaceutically acceptable salt thereof,
wherein:
each R¹ is separately selected from the group consisting of hydrogen and R^{1a}C(═O)— and R^{1a}C(═S)—;
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;
each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;
each R^{e}R^{f}N is separately selected, wherein R^{e} and R^{f} are each separately selected from the group consisting of hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;
each R^{x}R^{y}N is separately selected, wherein R^{x} and R^{y} are each separately selected from the group consisting of hydrogen, C₁₋₆alkylOC(═O)—, alkyl, alkylC(═O)—, aryl, arylalkyl, cycloalkyl, and heterocyclyl;
each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is 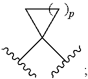
each R^{3a} is separately selected from the group consisting of hydrogen, and optionally substituted C₁₋₆alkyl;
each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;
each R^{4a}R^{4b}N is separately selected, wherein R^{4a} and R^{4b} are each separately selected from the group consisting of hydrogen, optionally substituted C₁₋₆alkyl, and aryl(CH₂)ₙ—;
each R^{5a} is separately selected from the group consisting of optionally substituted C₁₋₆alkyl, and aryl(CH₂)ₙ—;
each R^{6a} is separately selected from the group consisting of optionally substituted C₁₋₆alkyl, and aryl(CH₂)ₙ—;
X¹ is (C(R²)₂)_{q}, 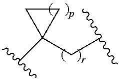
or X¹ is null;
Y¹ is selected from O (oxygen), S (sulfur), S(O), SO₂, NR², and C(R²)₂ with the proviso that when X¹ is null Y¹ is C(R²)₂;
X² is (C(R²)₂)_{q}, 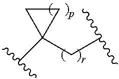
or X² is null;
Y² is selected from O (oxygen), S (sulfur), S(O), SO₂, NR², and C(R²)₂ with the proviso that when X² is null Y² is C(R²)₂;
each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups;
each R^{a}R^{b}N is separately selected, wherein R^{a} and R^{b} are each separately selected from the group consisting of hydrogen, C₂₋₆alkenyl, and C₁₋₆alkyl;
each Z is separately selected, wherein Z is selected from the group consisting of O (oxygen) and CH₂, or Z is null;
each A is separately selected from the group consisting of CR³ and N (nitrogen);
each L¹ is separately selected from the group consisting of 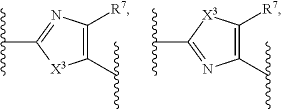
—C(═O)(CH₂)ₘOC(═O)—, —C(CF₃)₂NR^{2c}—, and 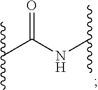
each X³ is separately selected from the group consisting of NH, NC₁₋₆alkyl, O (oxygen), and S (sulfur);
each m separately is 1 or 2;
each n separately is 0, 1 or 2;
each p separately is 1, 2, 3 or 4;
each q separately is 1, 2, 3, 4 or 5;
each r separately is 0, 1, 2, 3, or 4;
each R³ is separately selected from the group consisting of hydrogen; C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 9 halo and up to 5 hydroxy; and
each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo.

25. The compound of claim 24,
wherein:
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆ alkyl, aryl, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, (R^{c}R^{d}N)(CH═CH)ₘ—, (R^{c}R^{d}N)alkyl, (R^{c}R^{d}N)C(═O)—, C₁₋₆alkoxy optionally substituted with up to 5 halo, and C₁₋₆alkyl optionally substituted with up to 5 halo;
each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 5 halo, and C₁₋₆alkyl optionally substituted with up to 5 halo;
each R^{2a} is separately selected from the group consisting of hydrogen, C₁₋₆alkyl, aryl(CH₂)ₙ—, and heteroaryl(CH₂)ₙ—;
each R^{3a} is separately selected from the group consisting of hydrogen, and C₁₋₆alkyl;
each R^{3b} is separately selected from the group consisting of C₁₋₆alkyl, —(CH₂)ₙC(═O)NR^{4a}R^{4b}, —(CH₂)ₙC(═O)OR^{5a}, and —(CH₂)ₙC(═O)R^{6a};
each R^{4a}R^{4b}N is separately selected, wherein R^{4a} and R^{4b} are each separately selected from the group consisting of hydrogen, C₁₋₆alkyl, and aryl(CH₂)ₙ—;
each R^{5a} is separately selected from the group consisting of optionally substituted C₁₋₆alkyl, and aryl(CH₂)ₙ—;
each R^{6a} is separately selected from the group consisting of optionally substituted C₁₋₆alkyl, and aryl(CH₂)ₙ—;
X¹ is C(R²)₂, or X¹ is null;
Y¹ is selected from O (oxygen), S (sulfur), S(O), SO₂, and C(R²)₂ with the proviso that when X¹ is null Y¹ is C(R²)₂;
X² is C(R²)₂, or X² is null;
Y² is selected from O (oxygen), S (sulfur), S(O), SO₂, and C(R²)₂ with the proviso that when X² is null Y² is C(R²)₂;
each X³ is separately selected from the group consisting of NH, O (oxygen), and S (sulfur);
each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 5 halo, or optionally two vicinal R² and the carbons to which they are attached are together a fused three- to six-membered carbocyclic ring optionally substituted with up to two C₁₋₆alkyl groups;
each L¹ is separately selected from the group consisting of 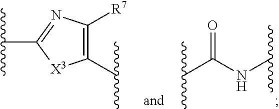
each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 5 halo and up to 5 hydroxy; and
each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 5 halo.

26. The compound of claim 24 having the structure of Formula IIIa: 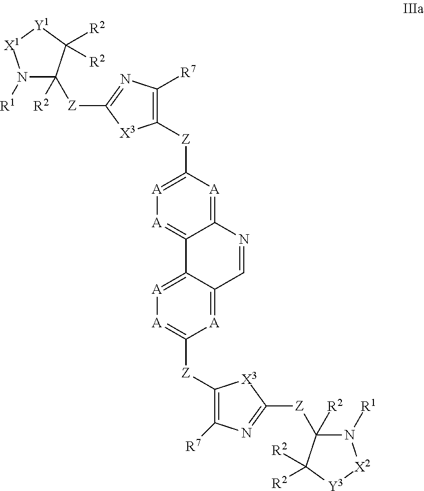
or a pharmaceutically acceptable salt thereof.

27. The compound of claim 26, wherein each Z is null.

28. The compound of claim 27 having the structure of Formula IIIb: 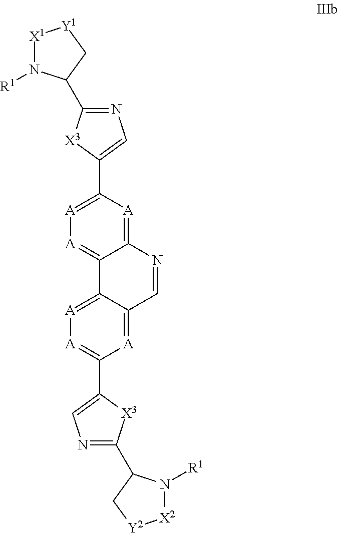
or a pharmaceutically acceptable salt thereof.

29. The compound of claim 28, wherein each R¹ is R^{1a}C(═O)—.

30. The compound of claim 29, wherein each R^{1a} is —CHR^{2a}NHR^{3b}.

31. The compound of claim 30, wherein each R^{2a} is C₁₋₆alkyl;
each R^{3b} is —C(═O)OR⁵; and
each R⁵ is C₁₋₆alkyl.

32. The compound of claim 24, having the structure: 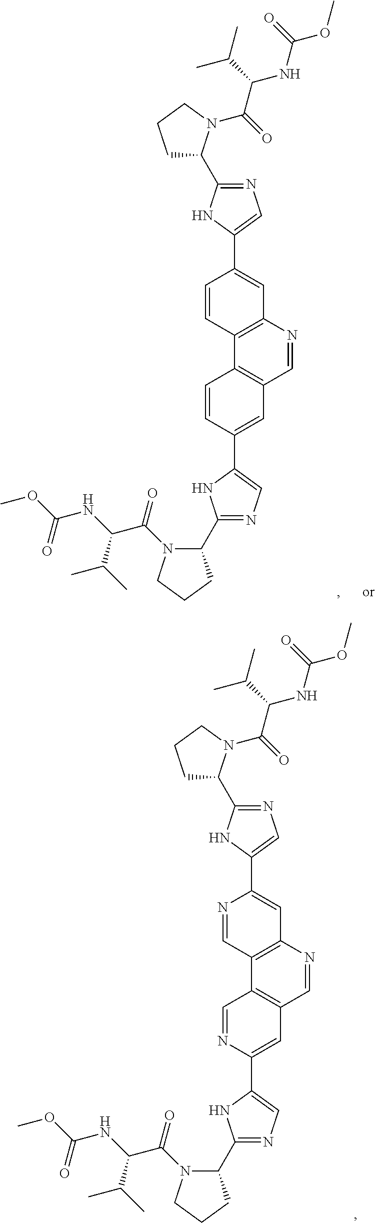
or a pharmaceutically acceptable salt thereof.

33. A compound having the structure of Formula IV: 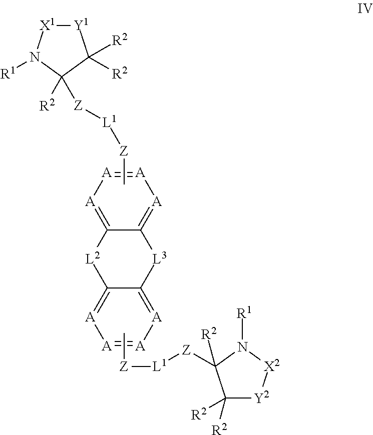
or a pharmaceutically acceptable salt thereof,
wherein:
each R¹ is separately selected from the group consisting of hydrogen and R^{1a}C(═O)— and R^{1a}C(═S)—;
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;
each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;
each R^{e}R^{f}N is separately selected, wherein R^{e} and R^{f} are each separately selected from the group consisting of hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;
each R^{x}R^{y}N is separately selected, wherein R^{x} and R^{y} are each separately selected from the group consisting of hydrogen, C₁₋₆alkylOC(═O)—, C₁₋₆alkyl, C₁₋₆alkylC(═O)—, aryl, arylalkyl, cycloalkyl, and heterocyclyl;
each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is 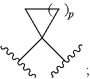
each R^{3a} is separately selected from the group consisting of hydrogen, and optionally substituted C₁₋₆alkyl;
each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;
each R^{4a}R^{4b}N is separately selected, wherein R^{4a} and R^{4b} are each separately selected from the group consisting of hydrogen, optionally substituted C₁₋₆alkyl, and aryl (CH₂)ₙ—;
each R^{5a} is separately selected from the group consisting of optionally substituted C₁₋₆alkyl, and aryl(CH₂)ₙ—;
each R^{6a} is separately selected from the group consisting of optionally substituted C₁₋₆alkyl, and aryl(CH₂)ₙ—;
X¹ is (C(R²)₂)_{q}, 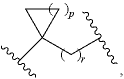
or X¹ is null;
Y¹ is selected from O (oxygen), S (sulfur), S(O), SO₂, NR², and C(R²)₂ with the proviso that when X¹ is null Y¹ is C(R²)₂;
X² is (C(R²)₂)_{q}, 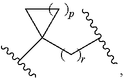
or X² is null;
Y² is selected from O (oxygen), S (sulfur), S(O), SO₂, NR², and C(R²)₂ with the proviso that when X² is null Y² is C(R²)₂;
each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups;
each R^{a}R^{b}N is separately selected, wherein R^{a} and R^{b} are each separately selected from the group consisting of hydrogen, C₂₋₆alkenyl, and C₁₋₆alkyl;
each Z is separately selected, wherein Z is selected from the group consisting of O (oxygen) and CH₂, or Z is null;
each A is separately selected from the group consisting of CR³ and N (nitrogen);
each L¹ is separately selected from the group consisting of 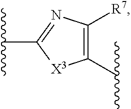
—C(═O)(CH₂)ₘOC(═O)—, —C(CF₃)₂NR^{2c}—, and 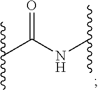
each X₃ is separately selected from the group consisting of NH, NC₁₋₆alkyl, O (oxygen), and S (sulfur);
L² is selected from the group consisting of —C(═O)—, —(CH₂CH₂)—, —(CH₂O)—, —(CH₂S)—, —(CH═CH)—, —(CH═N)—, —NH—, O (oxygen), S (sulfur), and —CH₂—;
L³ is selected from the group consisting of 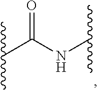
—(NR⁹)—, O (oxygen), S (sulfur), and —CH₂—;
R⁹ is selected from the group consisting of hydrogen and —C(═O)R^{9a};
R^{9a} is selected from the group consisting of —NR^{9b}R^{9c}, —OR^{9d}, C₁₋₆alkoxy optionally substituted with up to 9 halo, C₁₋₆alkyl optionally substituted with up to 9 halo, and optionally substituted aryl;
R^{9b} is selected from the group consisting of hydrogen, C₁₋₆alkyl optionally substituted with up to 9 halo, and optionally substituted aryl;
R^{9c} is selected from the group consisting of C₁₋₆alkyl optionally substituted with up to 9 halo, and optionally substituted aryl;
R^{9d} is selected from the group consisting of C₁₋₆alkyl optionally substituted with up to 9 halo, and optionally substituted aryl;
each m separately is 1 or 2;
each n separately is 0, 1 or 2;
each p separately is 1, 2, 3 or 4;
each q separately is 1, 2, 3, 4 or 5;
each r separately is 0, 1, 2, 3, or 4;
each R³ is separately selected from the group consisting of hydrogen, C₁₋₆alkoxy, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 9 halo and up to 5 hydroxy; and
each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo;
provided that the compound is not selected from the group consisting of:

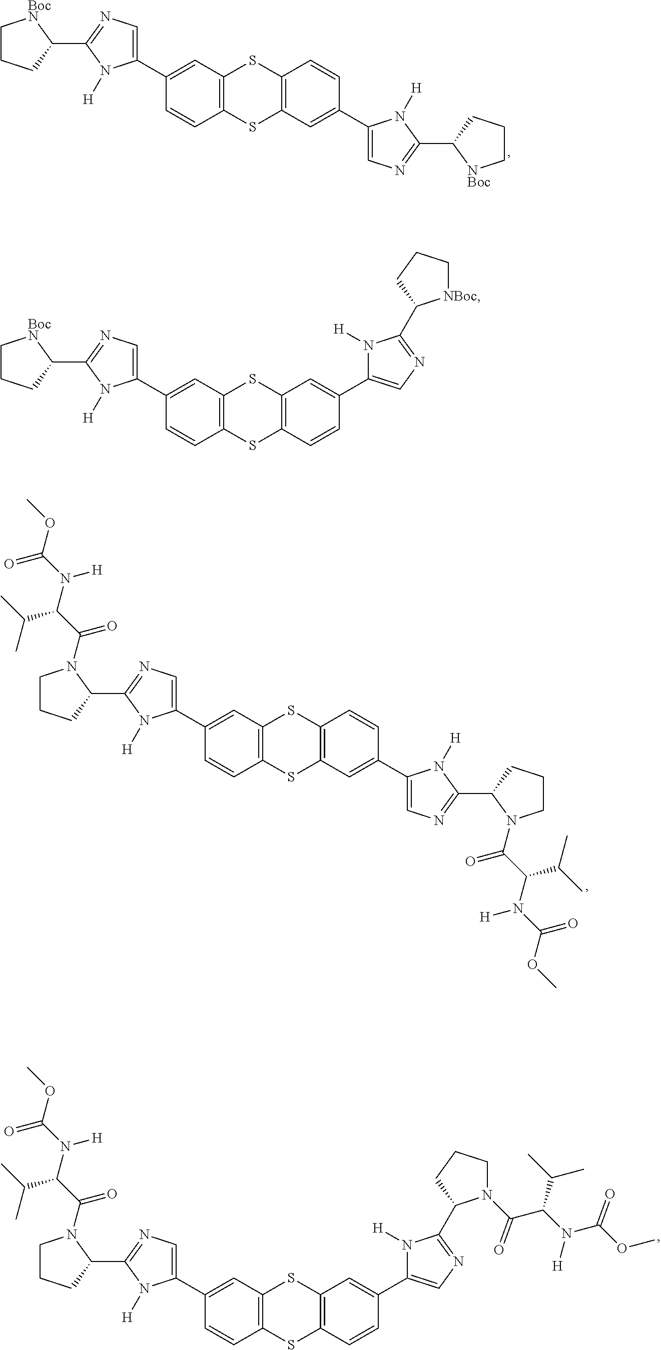

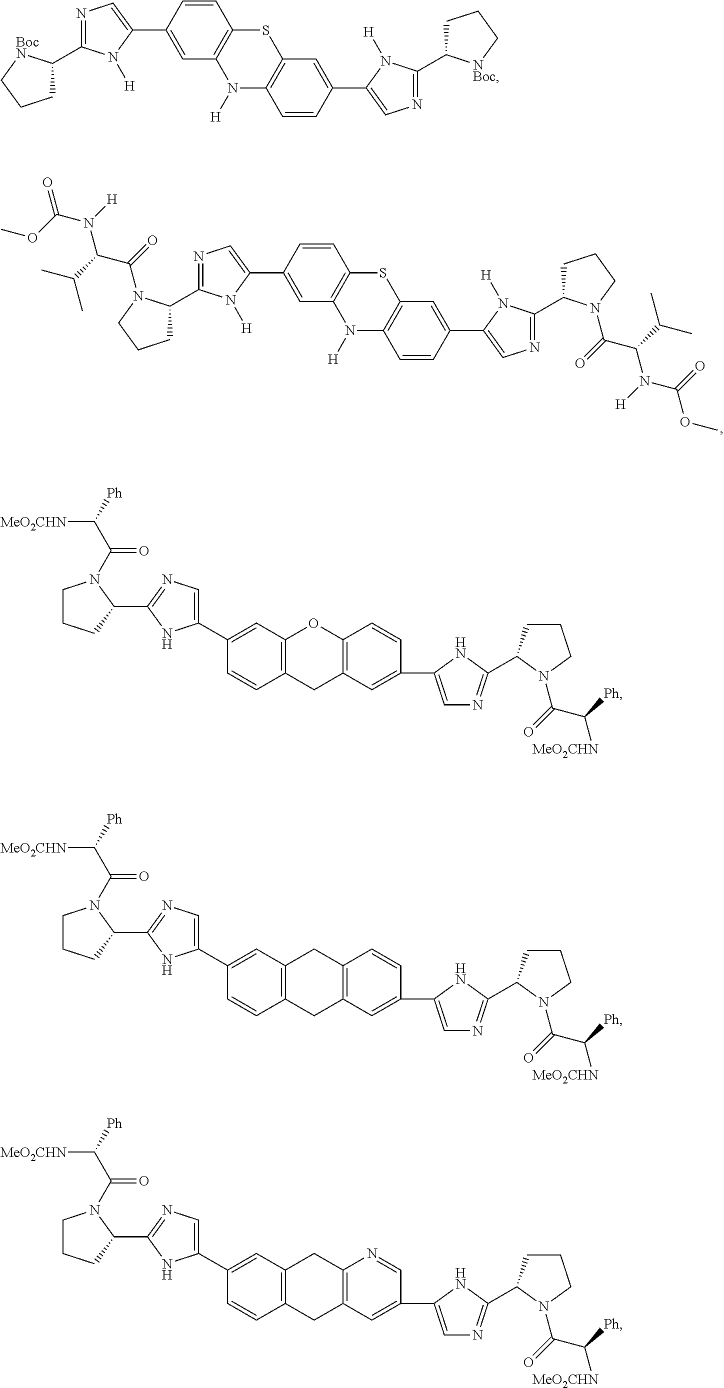

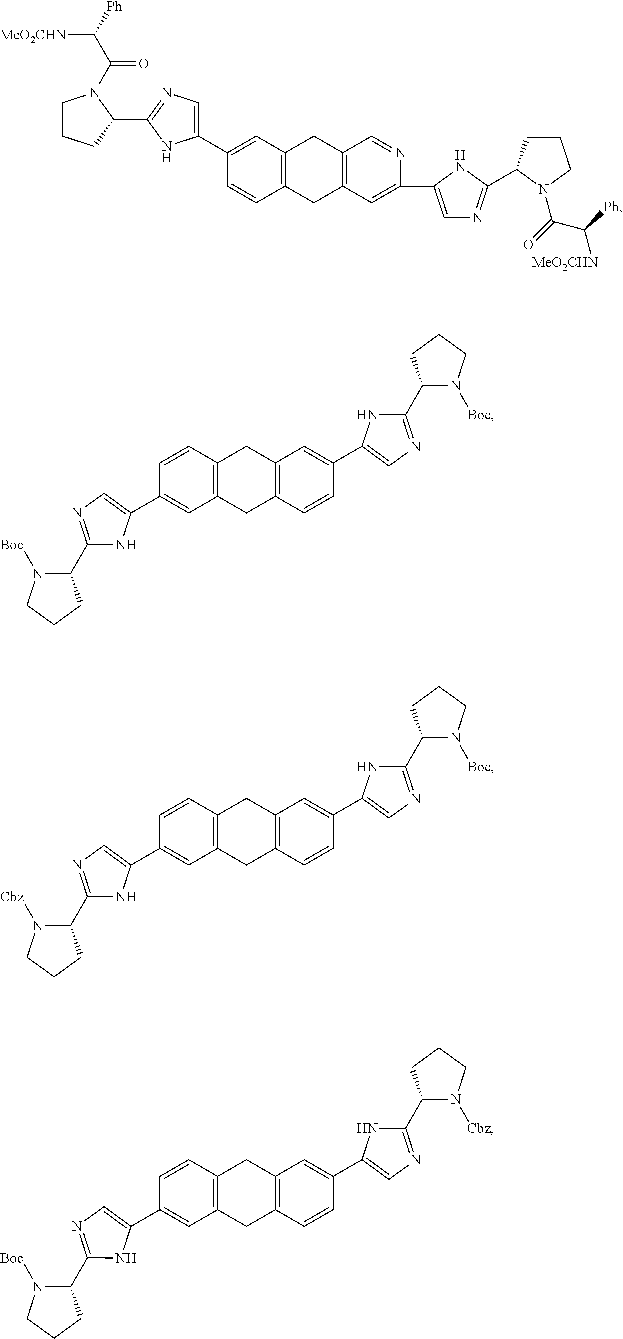

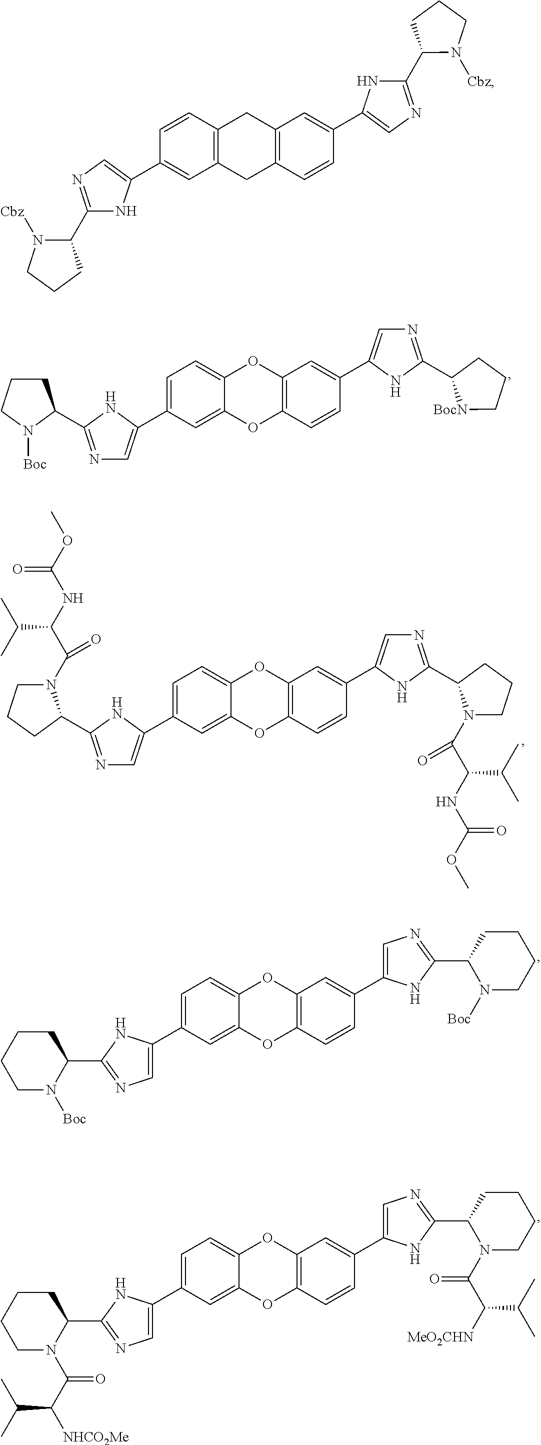

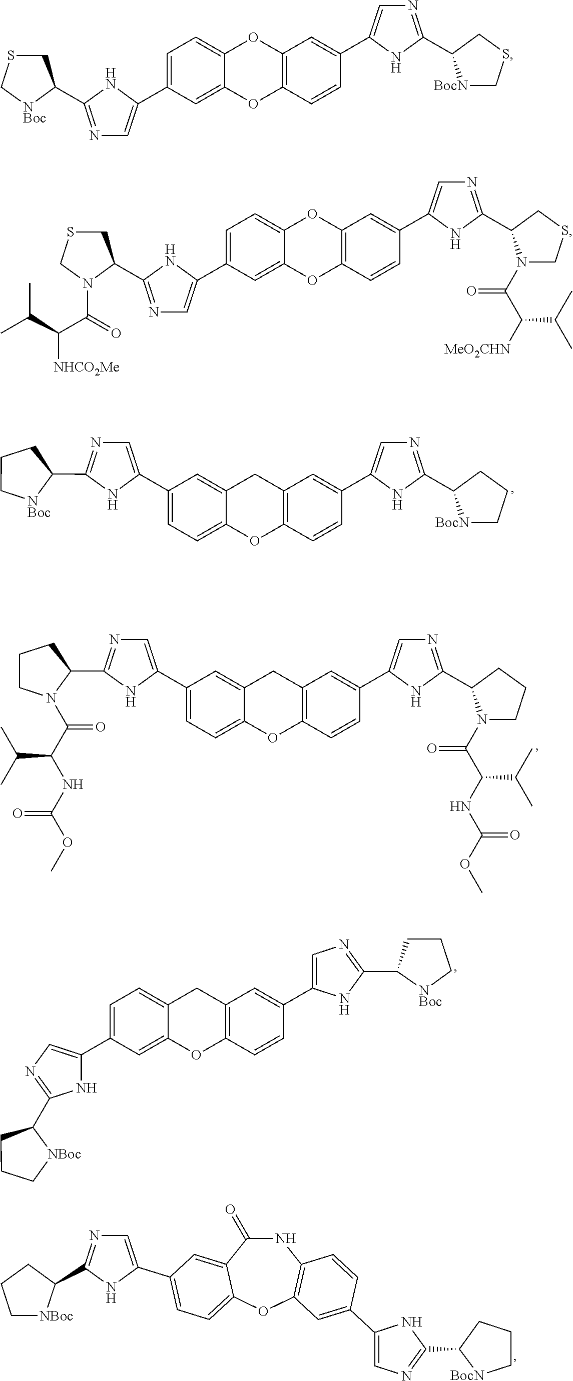

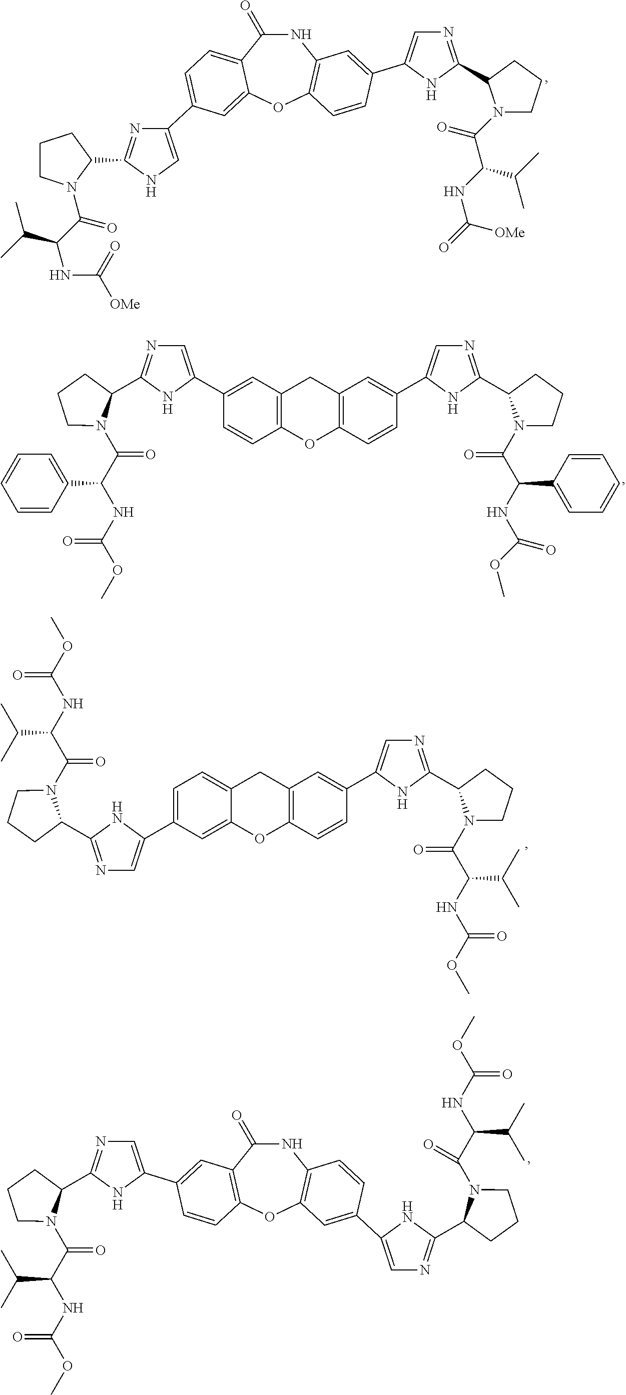

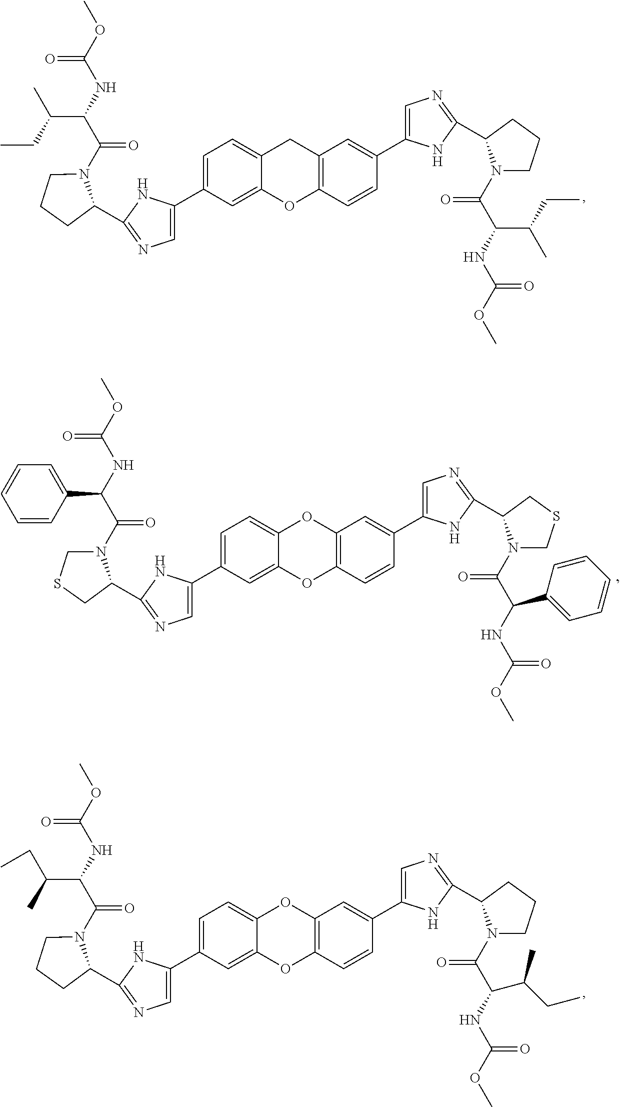

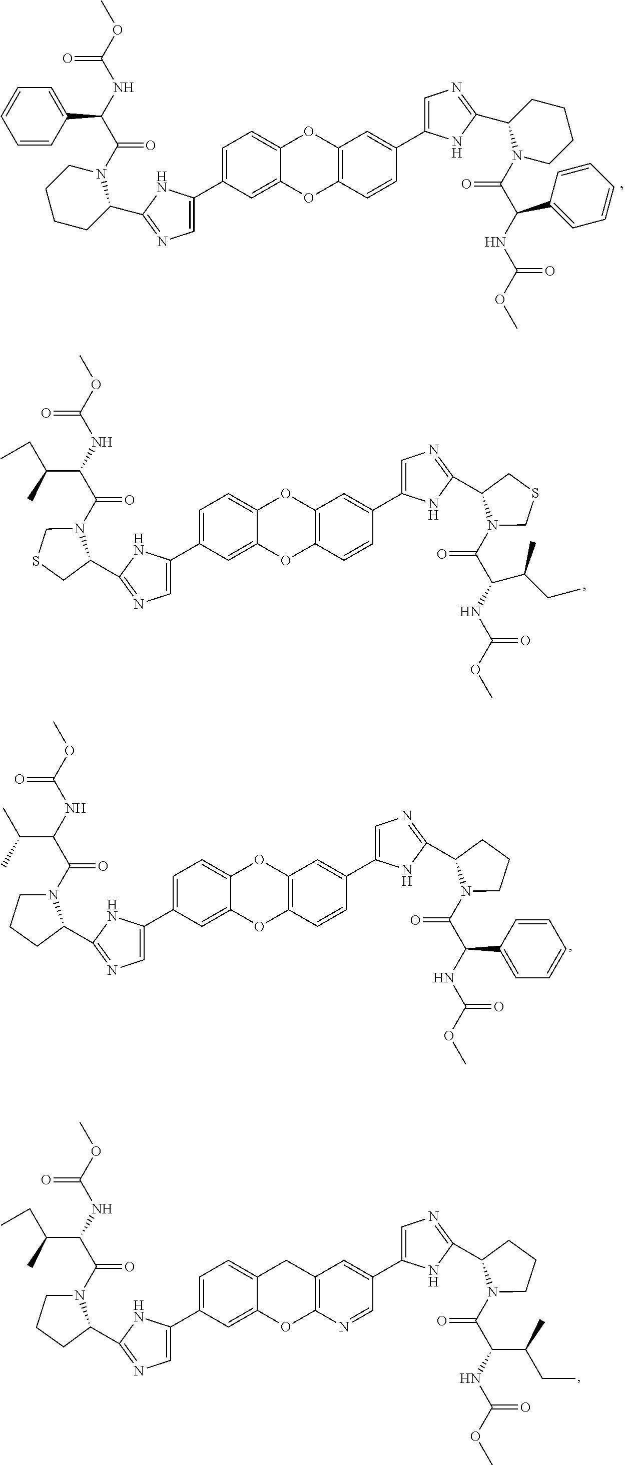

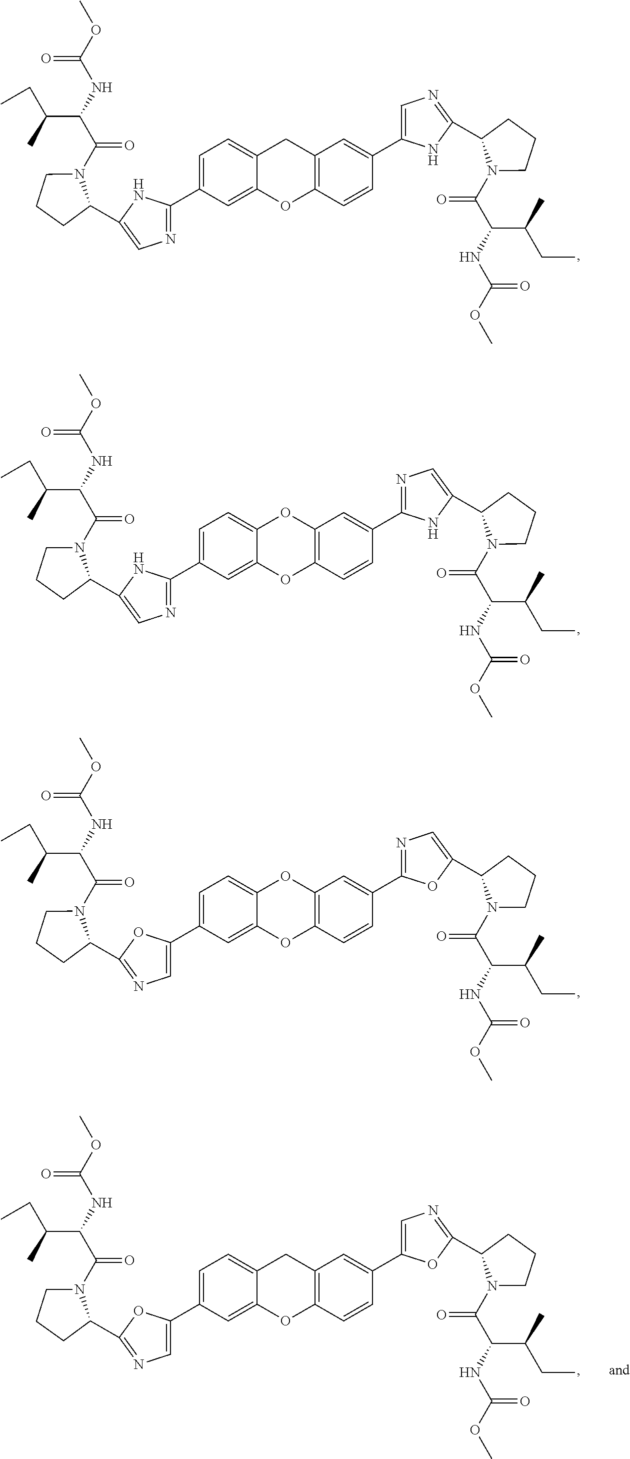

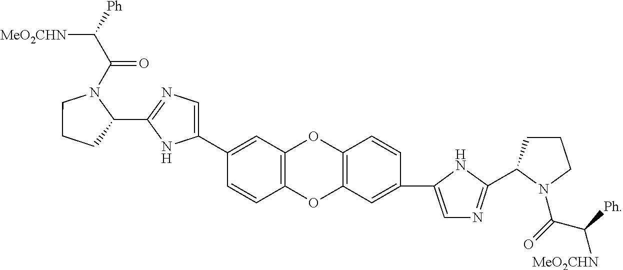

34. The compound of claim 33,
wherein:
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, (R^{c}R^{d}N)(CH═CH)ₘ—, (R^{c}R^{d}N)alkyl, (R^{c}R^{d}N)C(═O)—, C₁₋₆alkoxy optionally substituted with up to 5 halo, and C₁₋₆alkyl optionally substituted with up to 5 halo;
each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f})alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 5 halo, and C₁₋₆alkyl optionally substituted with up to 5 halo;
each R^{2a} is separately selected from the group consisting of hydrogen, C₁₋₆alkyl, aryl(CH₂)ₙ—, and heteroaryl(CH₂)ₙ—;
each R^{3a} is separately selected from the group consisting of hydrogen, and C₁₋₆alkyl;
each R^{3b} is separately selected from the group consisting of C₁₋₆alkyl, —(CH₂)ₙC(═O)NR^{4a}R^{4b}, —(CH₂)ₙC(═O)OR^{5a}, and —(CH₂)ₙC(═O)R^{6a};
each R^{4a}R^{4b}N is separately selected, wherein R^{4a} and R^{4b} are each separately selected from the group consisting of hydrogen, C₁₋₆alkyl, and aryl(CH₂)ₙ—;
each R^{5a} is separately selected from the group consisting of C₁₋₆alkyl, and aryl (CH₂)ₙ—;
each R^{6a} is separately selected from the group consisting of C₁₋₆alkyl, and aryl(CH₂)ₙ—;
X¹ is C(R²)₂, or X¹ is null;
Y¹ is selected from O (oxygen), S (sulfur), S(O), SO₂, and C(R²)₂ with the proviso that when X¹ is null Y¹ is C(R²)₂;
X² is C(R²)₂, or X² is null;
Y² is selected from O (oxygen), S (sulfur), S(O), SO₂, and C(R²)₂ with the proviso that when X² is null Y² is C(R²)₂;
each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 5 halo, or optionally two vicinal R² and the carbons to which they are attached are together a fused three- to six-membered carbocyclic ring optionally substituted with up to two C₁₋₆alkyl groups;
each L¹ is separately selected from the group consisting of 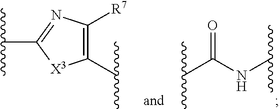
R^{9a} is selected from the group consisting of —NR^{9b}R^{9c}, —OR^{9d}, C₁₋₆alkyl optionally substituted with up to 5 halo, and optionally substituted aryl;
R^{9b} is selected from the group consisting of hydrogen, C₁₋₆alkyl optionally substituted with up to 5 halo, and optionally substituted aryl;
R^{9c} is selected from the group consisting of C₁₋₆alkyl optionally substituted with up to 5 halo, and optionally substituted aryl;
R^{9d} is selected from the group consisting of C₁₋₆alkyl optionally substituted with up to 5 halo, and optionally substituted aryl;
each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 5 halo and up to 5 hydroxy; and
each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 5 halo.

35. The compound of claim 33, wherein each Z is null.

36. The compound of claim 35 having the formula, 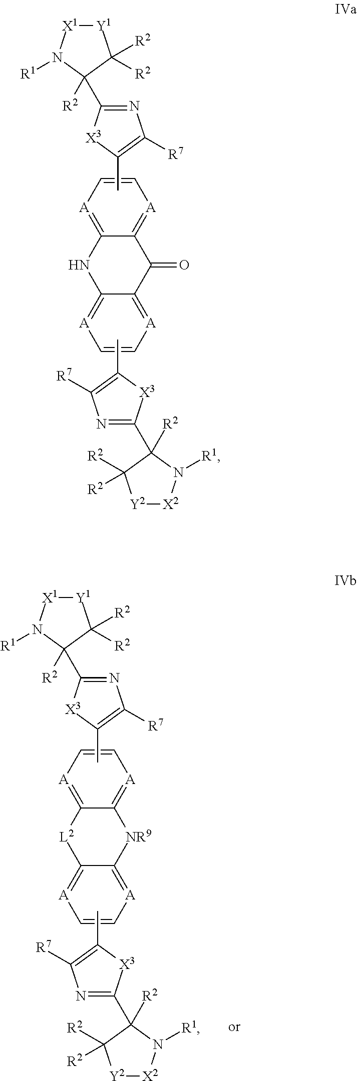 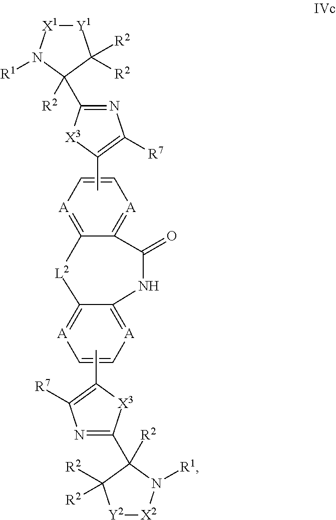
or pharmaceutically acceptable salts thereof.

37. The compound of claim 36, wherein each R¹ is R^{1a}C(═O)—.

38. The compound of claim 37, wherein each R^{1a} is —CHR^{2a}NHR^{3b}.

39. The compound of claim 38, wherein each R^{2a} is C₁₋₆alkyl;
each R^{3b} is —C(═O)OR⁵; and
each R⁵ is C₁₋₆alkyl.

40. The compound of claim 33, having the structure: 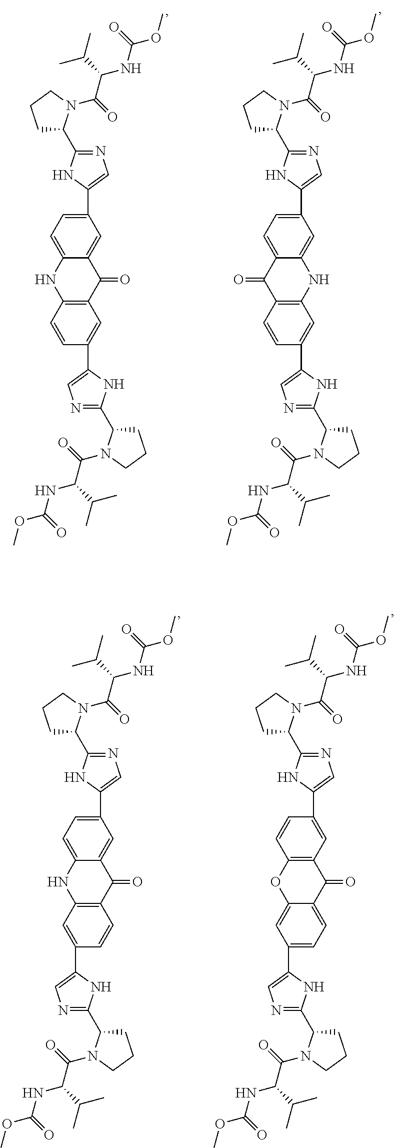 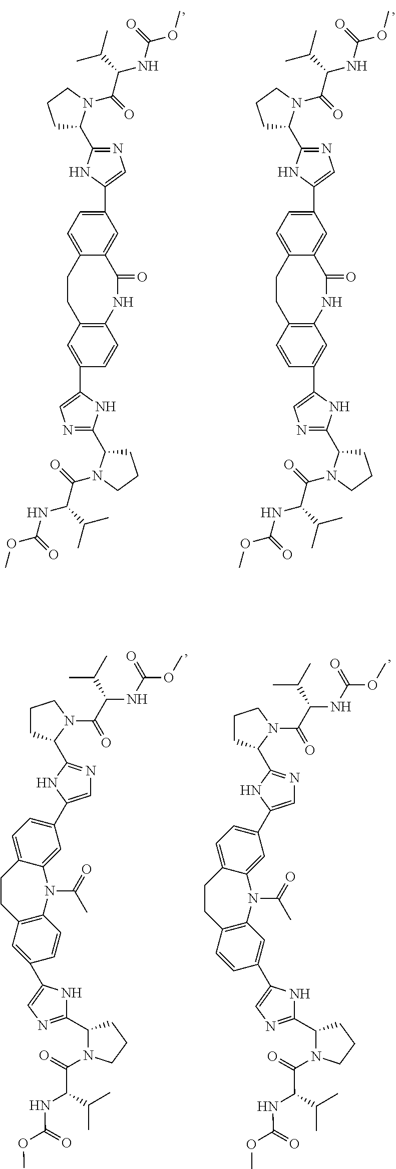 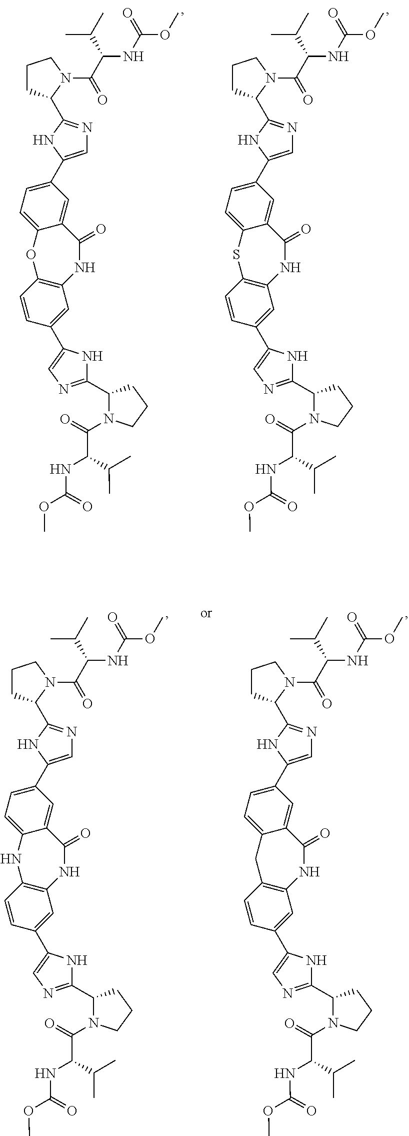
or a pharmaceutically acceptable salt thereof.

41. A compound having the structure of Formula V: 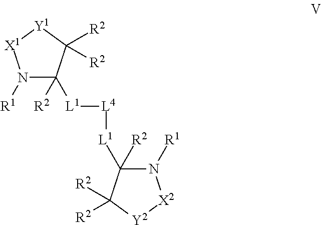
or a pharmaceutically acceptable salt thereof,
wherein:
each R¹ is separately selected from the group consisting of hydrogen and R^{1a}C(═O)— and R^{1a}C(═S)—;
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;
each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;
each R^{e}R^{f}N is separately selected, wherein R^{e} and R^{f} are each separately selected from the group consisting of hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;
each R^{x}R^{y}N is separately selected, wherein R^{x} and R^{y} are each separately selected from the group consisting of hydrogen, C₁₋₆alkylOC(═O)—, C₁₋₆alkyl, C₁₋₆alkylC(═O)—, aryl, arylalkyl, cycloalkyl, and heterocyclyl;
each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is 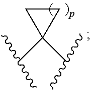
each R^{3a} is separately selected from the group consisting of hydrogen, and optionally substituted C₁₋₆alkyl;
each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;
each R^{4a}R^{4b}N is separately selected, wherein R^{4a} and R^{4b} are each separately selected from the group consisting of hydrogen, optionally substituted C₁₋₆alkyl, and aryl(CH₂)ₙ—;
each R^{5a} is separately selected from the group consisting of optionally substituted C₁₋₆alkyl, and aryl(CH₂)ₙ—;
each R^{6a} is separately selected from the group consisting of optionally substituted C₁₋₆alkyl, and aryl(CH₂)ₙ—;
X¹ is (C(R²)₂)_{q}, 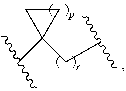
or X¹ is null;
Y¹ is selected from O (oxygen), S (sulfur), S(O), SO₂, NR², and C(R²)₂ with the proviso that when X¹ is null is C(R²)₂;
X² is (C(R²)₂)_{q}, 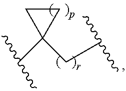
or X² is null;
Y² is selected from O (oxygen), S (sulfur), S(O), SO₂, NR², and C(R²)₂ with the proviso that when X² is null Y² is C(R²)₂;
each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups;
each R^{a}R^{b}N is separately selected, wherein R^{a} and R^{b} are each separately selected from the group consisting of hydrogen, C₂₋₆alkenyl, and C₁₋₆alkyl;
each A is separately selected from the group consisting of CR³ and N (nitrogen);
each L¹ is separately selected from the group consisting of 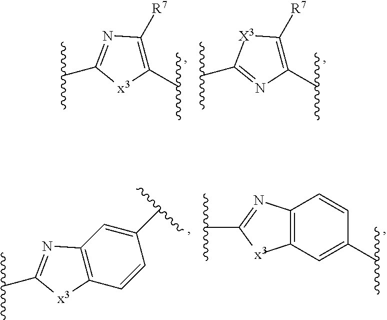
—C(═O)(CH₂)ₘOC(═O)—, —C(CF₃)₂NR^{2c}—, and 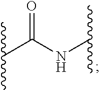
each X³ is separately selected from the group consisting of NH, NC₁₋₆alkyl, O (oxygen), and S (sulfur);
L⁴ is selected from the group consisting of 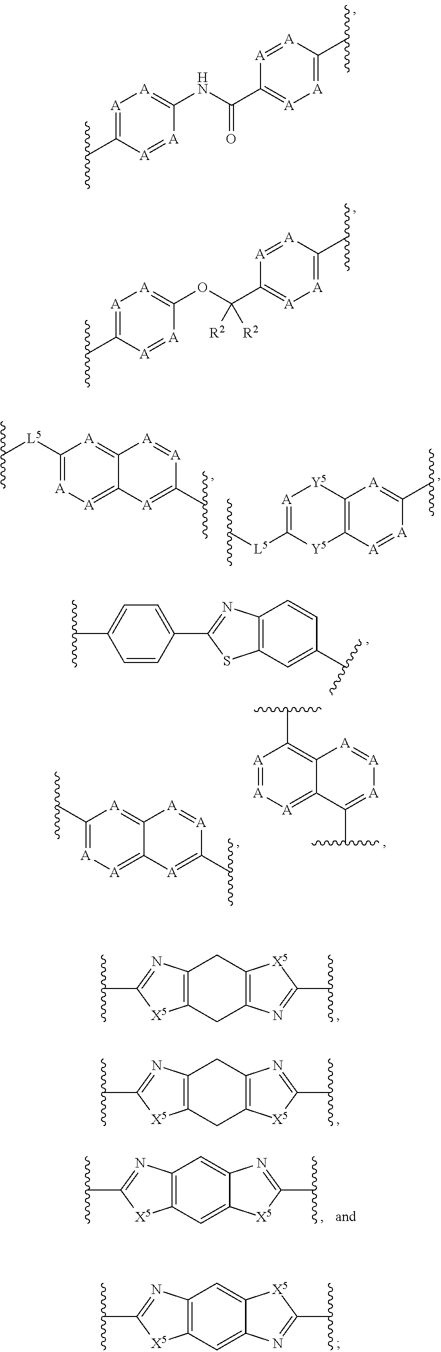
L⁵ is selected from the group consisting of 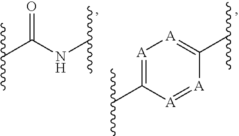
and —(CH═CH)—;
each X⁵ is separately selected from the group consisting of —NH—, O (oxygen), S (sulfur), and —CH₂—,
each Y⁵ is separately selected from the group consisting of O (oxygen), S (sulfur), S(O), SO₂, NR², and C(R²)₂;
each m separately is 1 or 2;
each n separately is 0, 1 or 2;
each p separately is 1, 2, 3 or 4;
each q separately is 1, 2, 3, 4 or 5;
each r separately is 0, 1, 2, 3, or 4;
each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 9 halo and up to 5 hydroxy; and
each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo;
provided that the compound is not selected from the group consisting of:

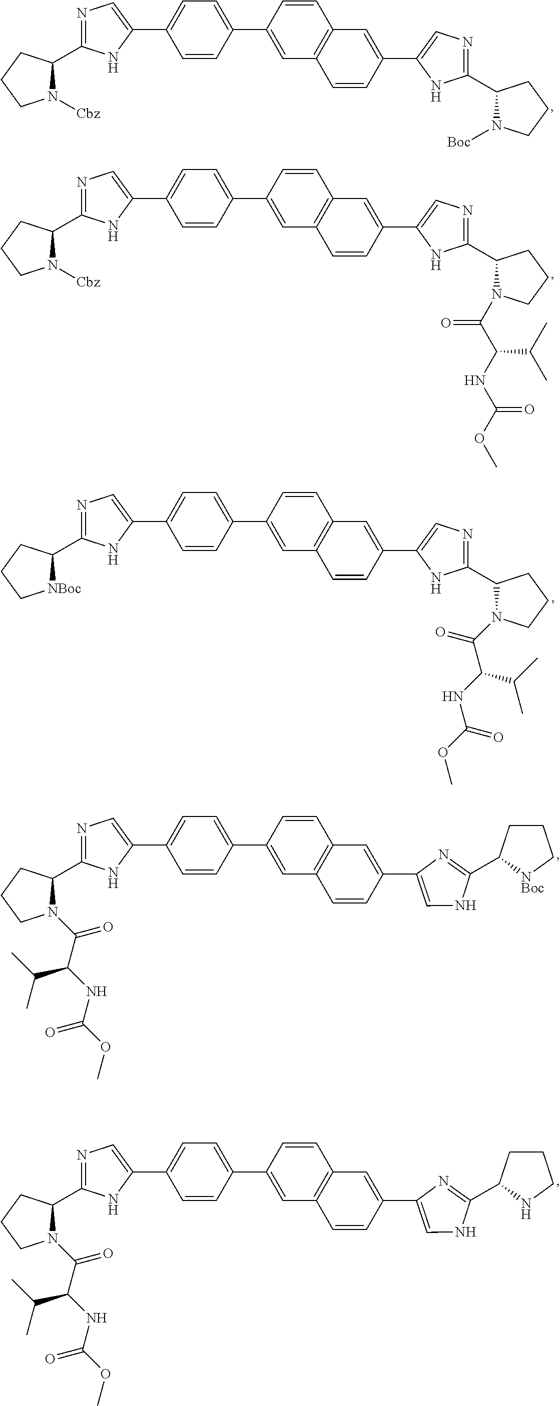

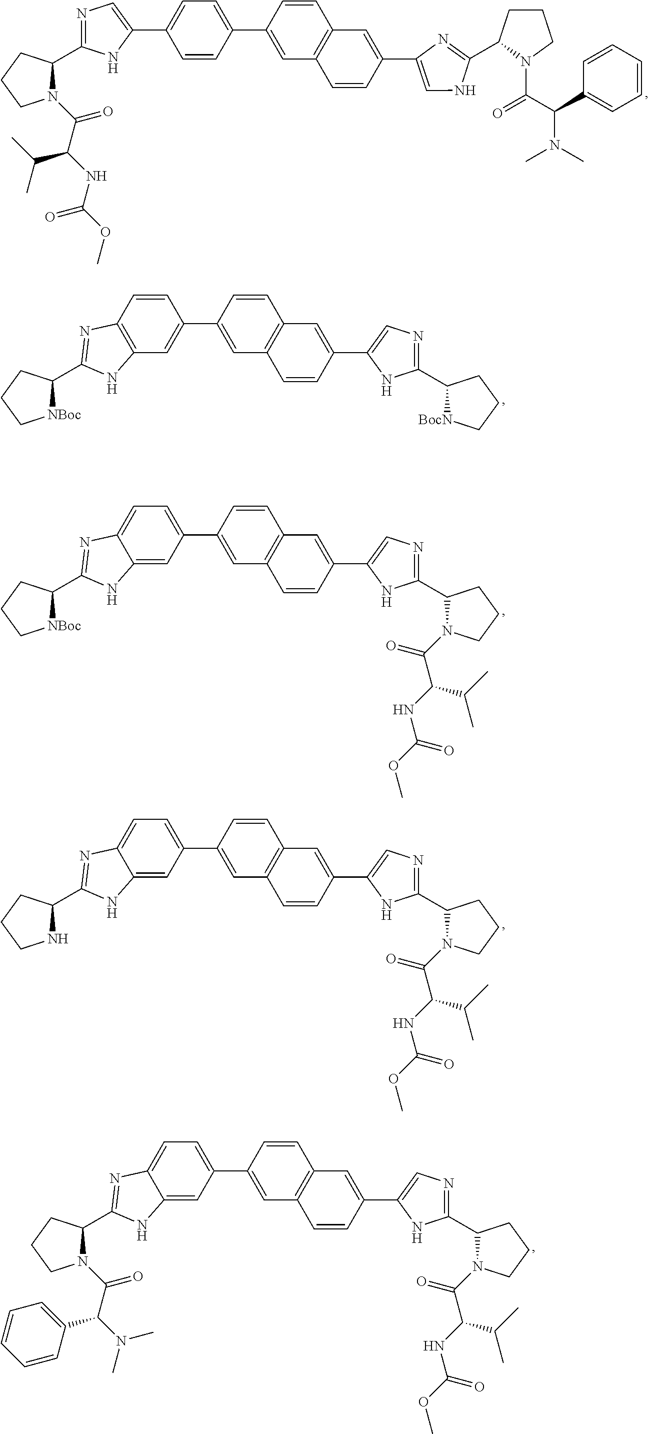

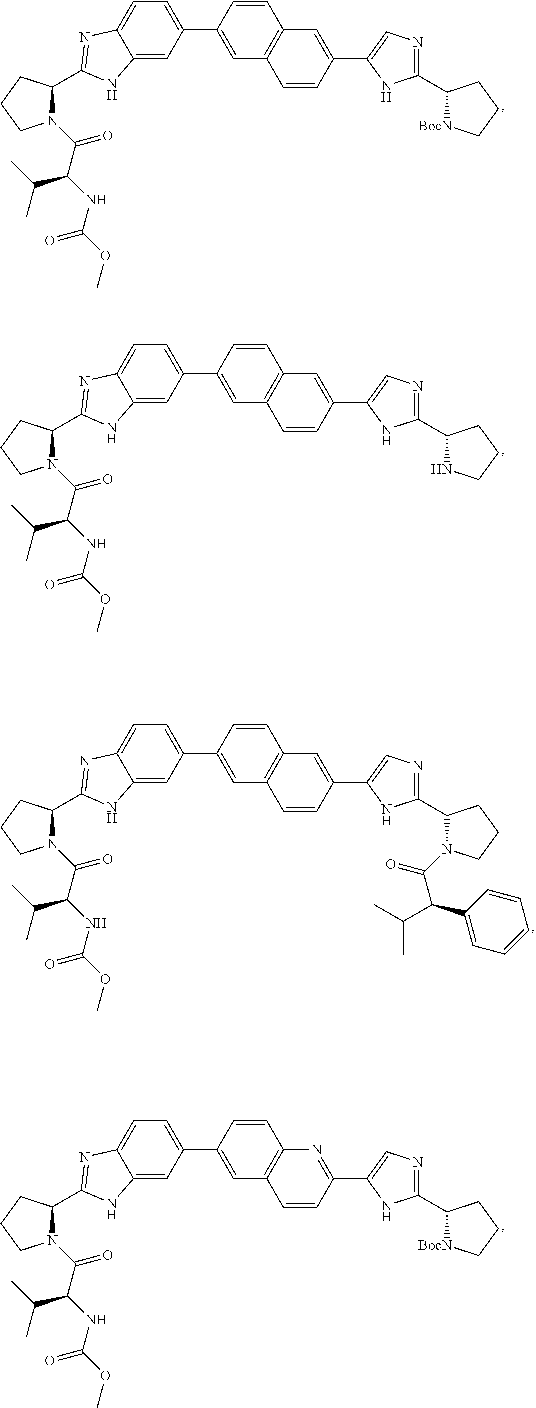

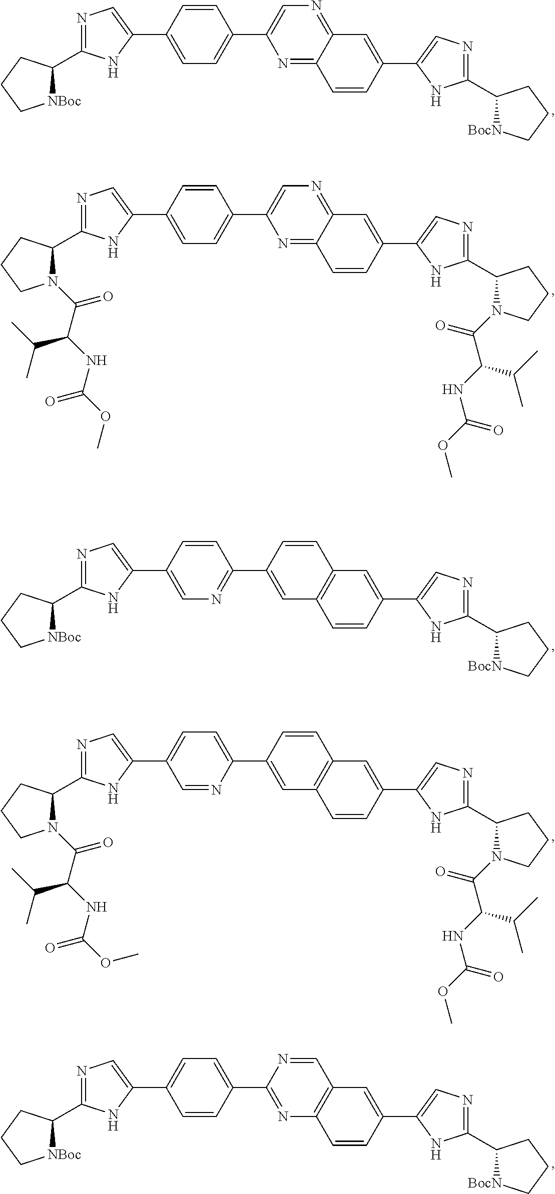

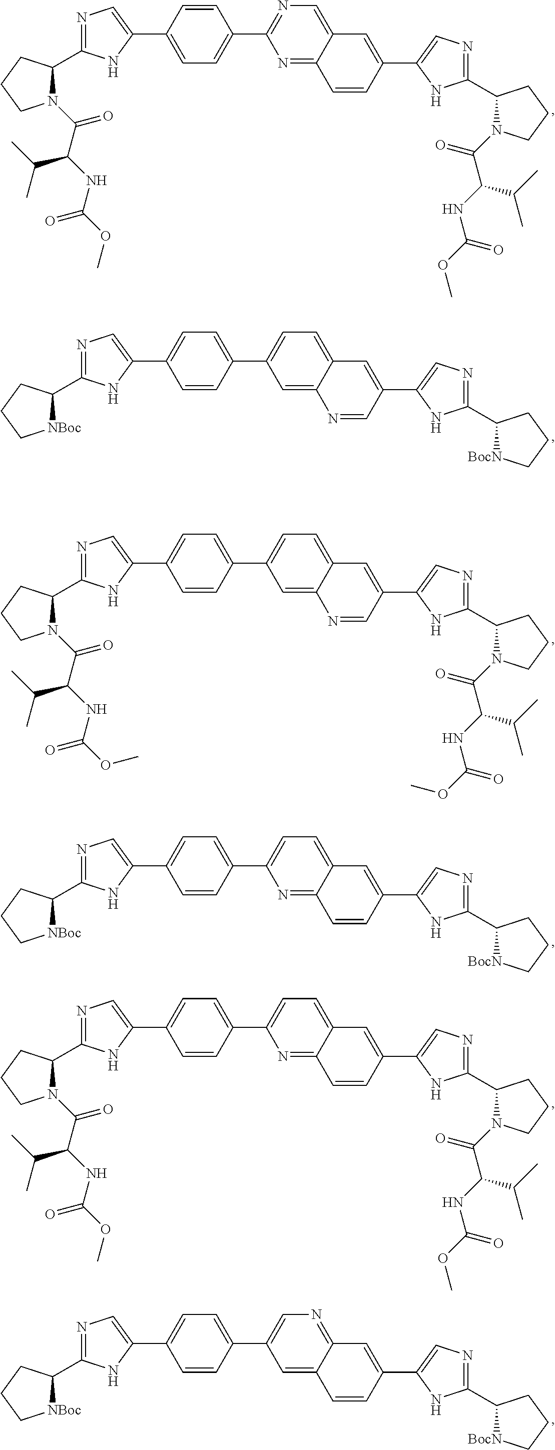

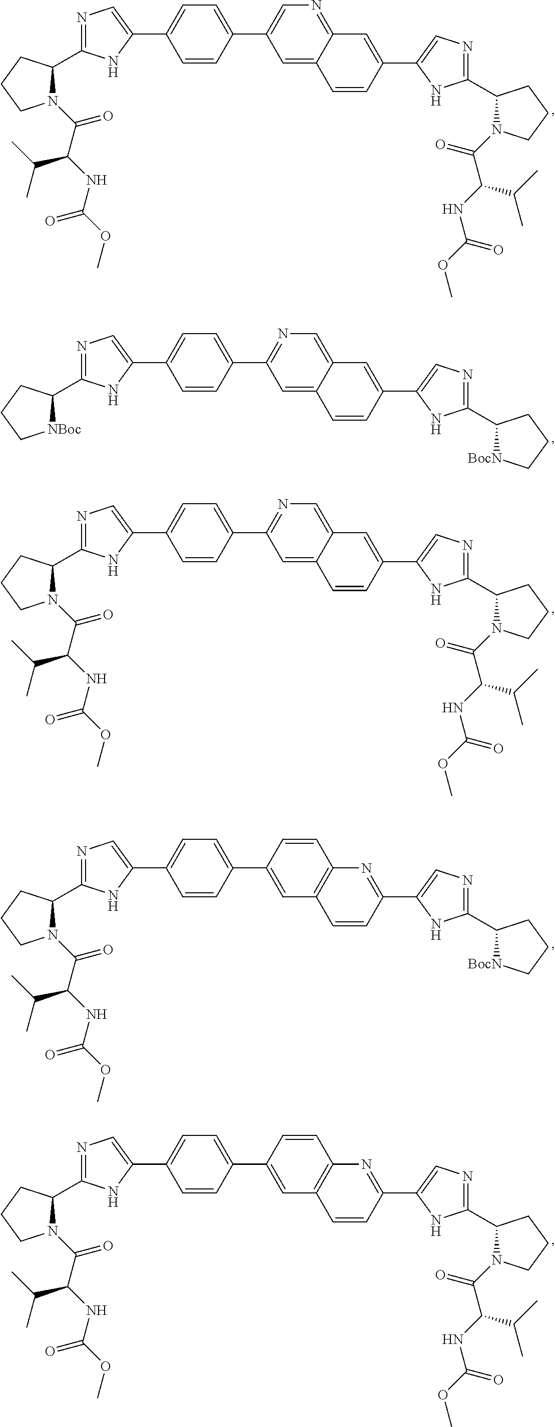

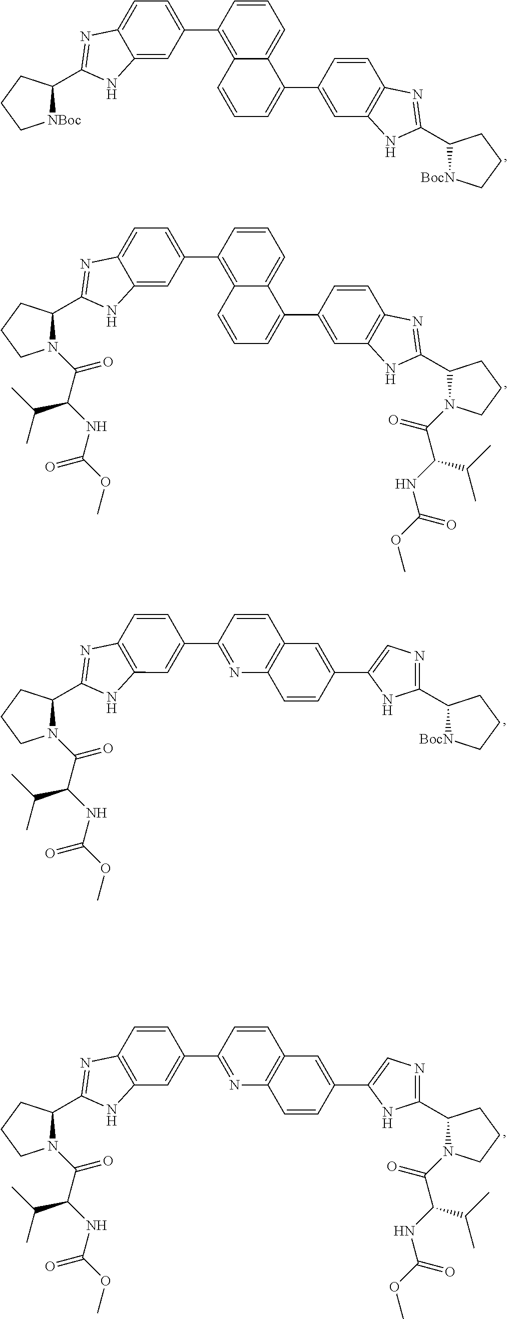

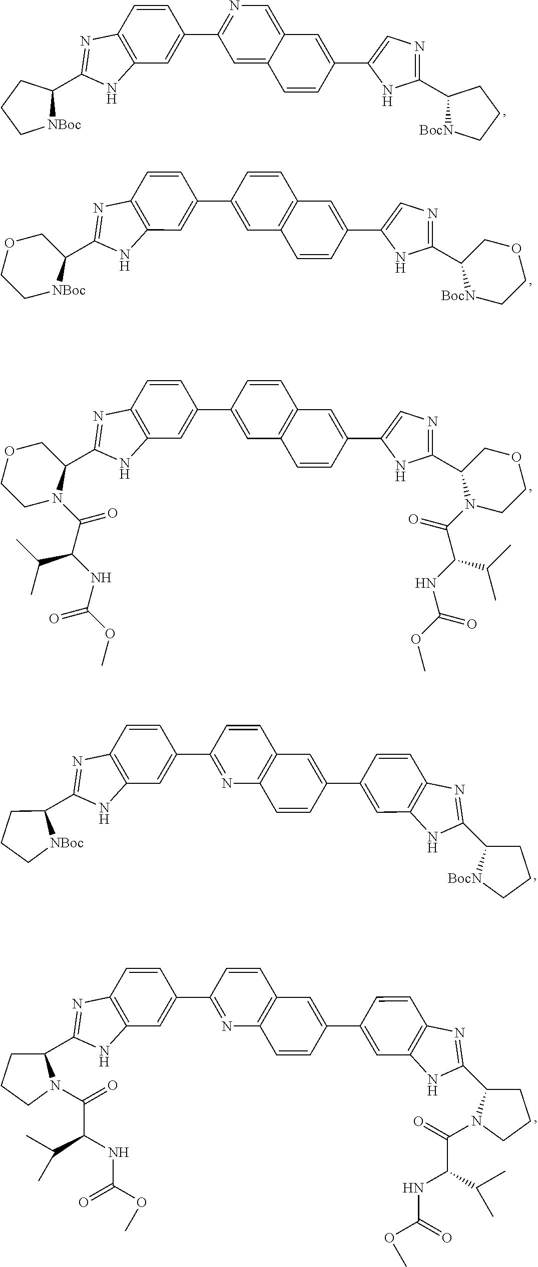

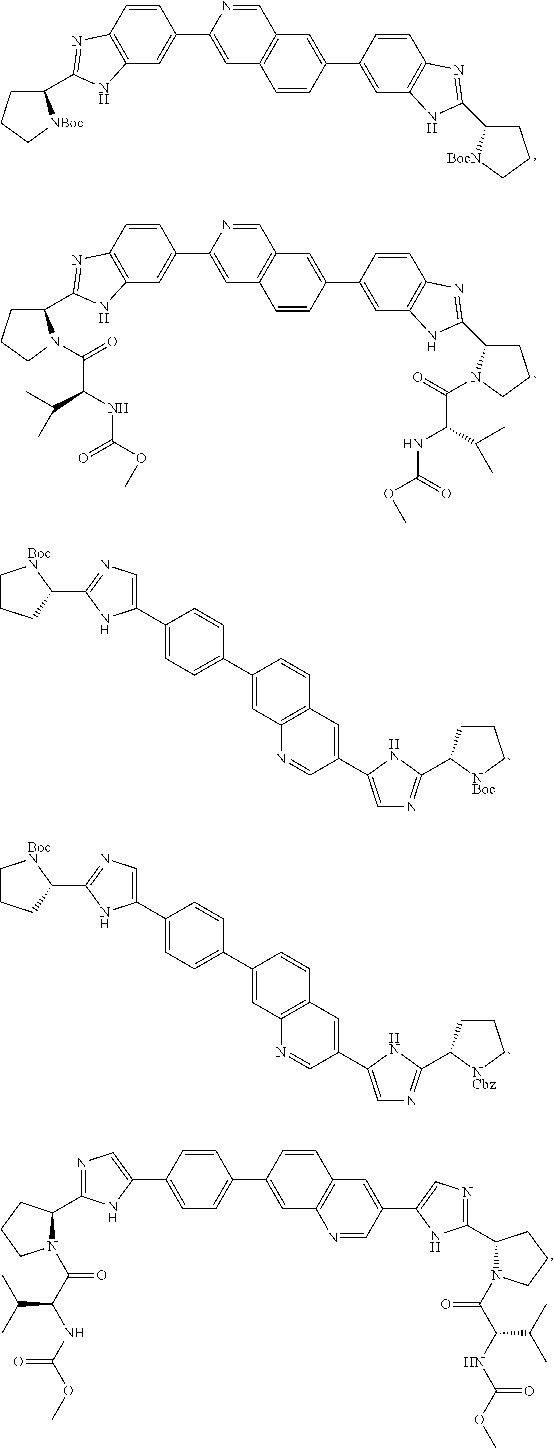

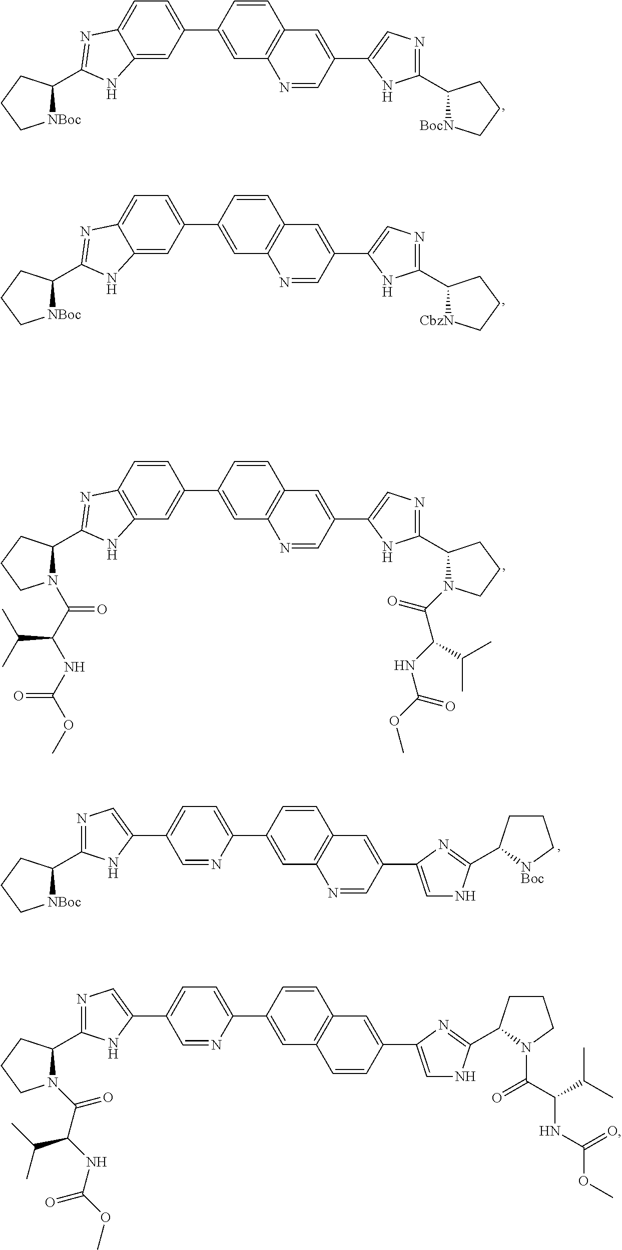

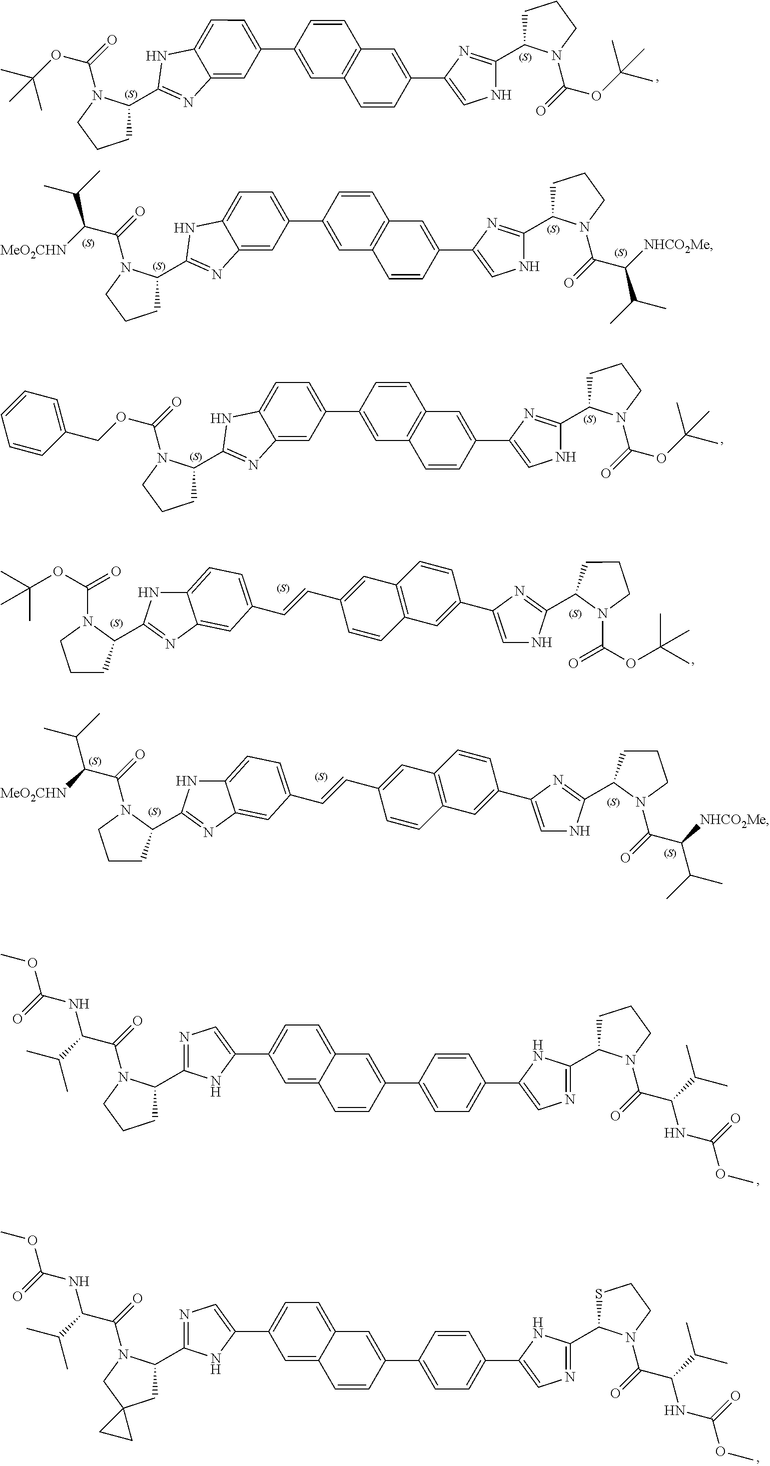

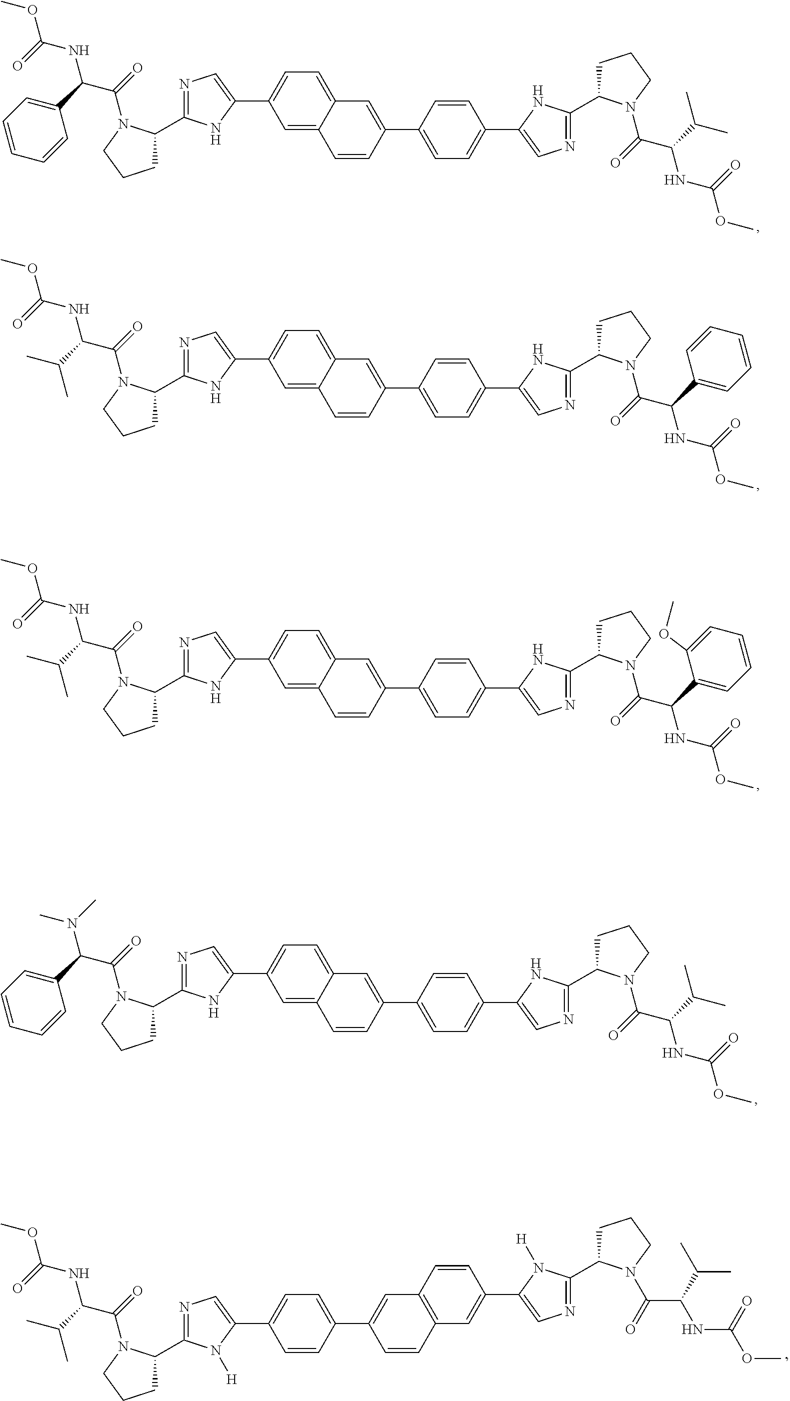

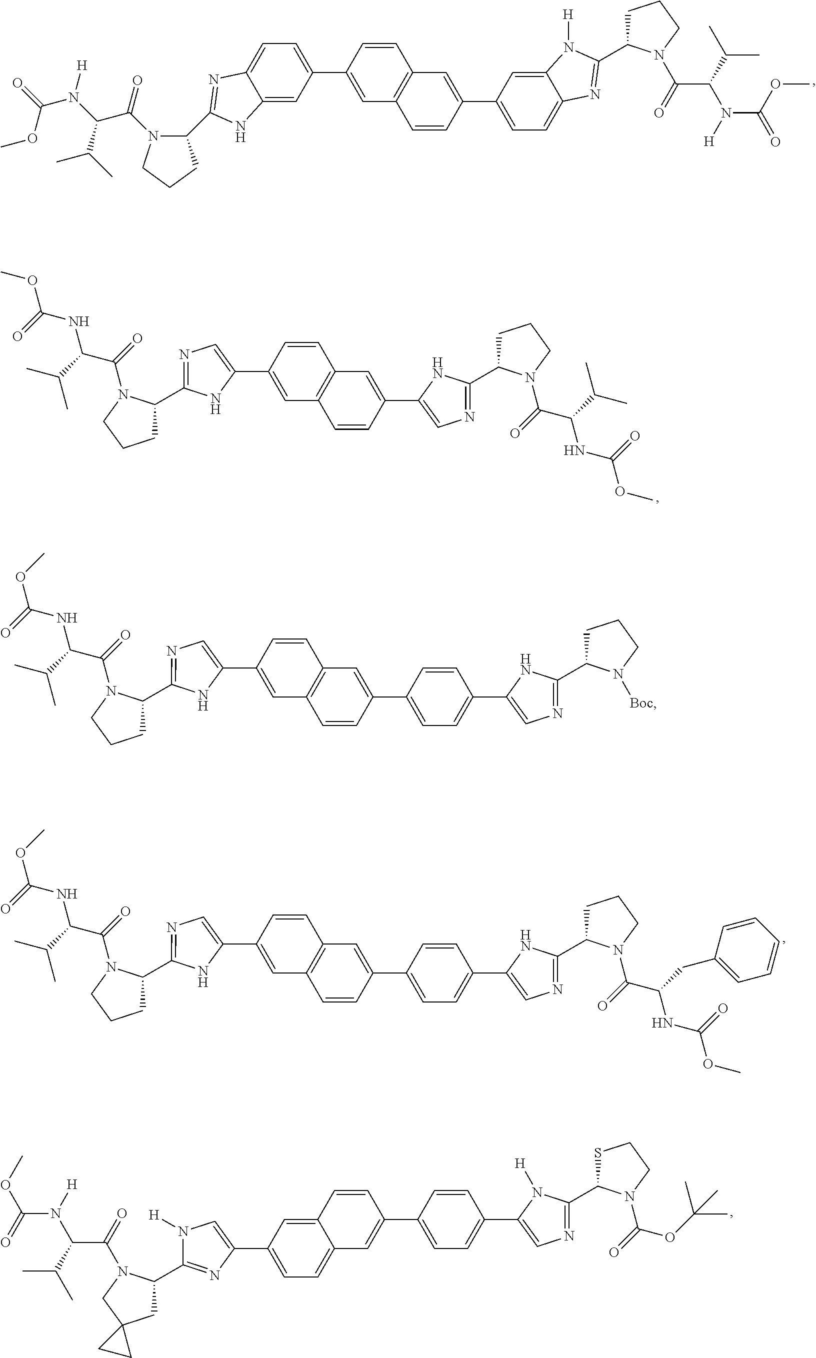

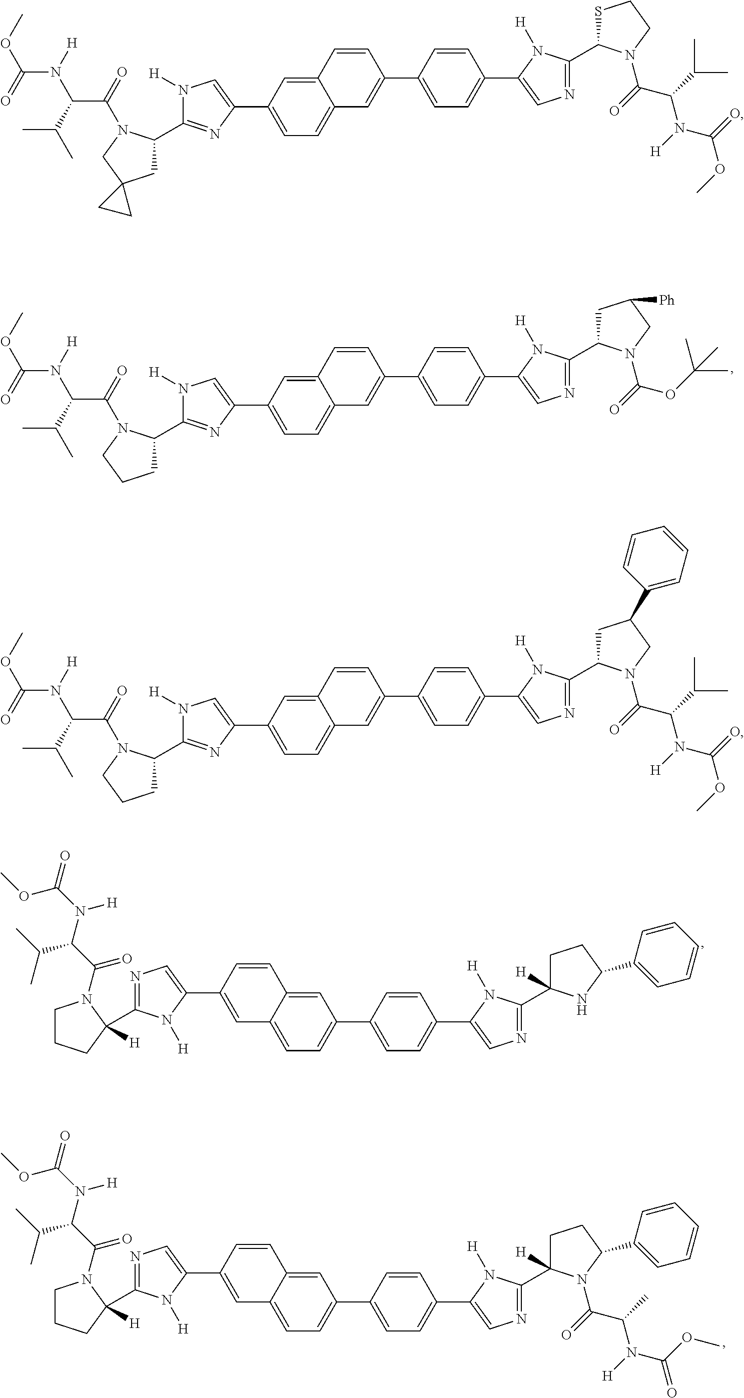

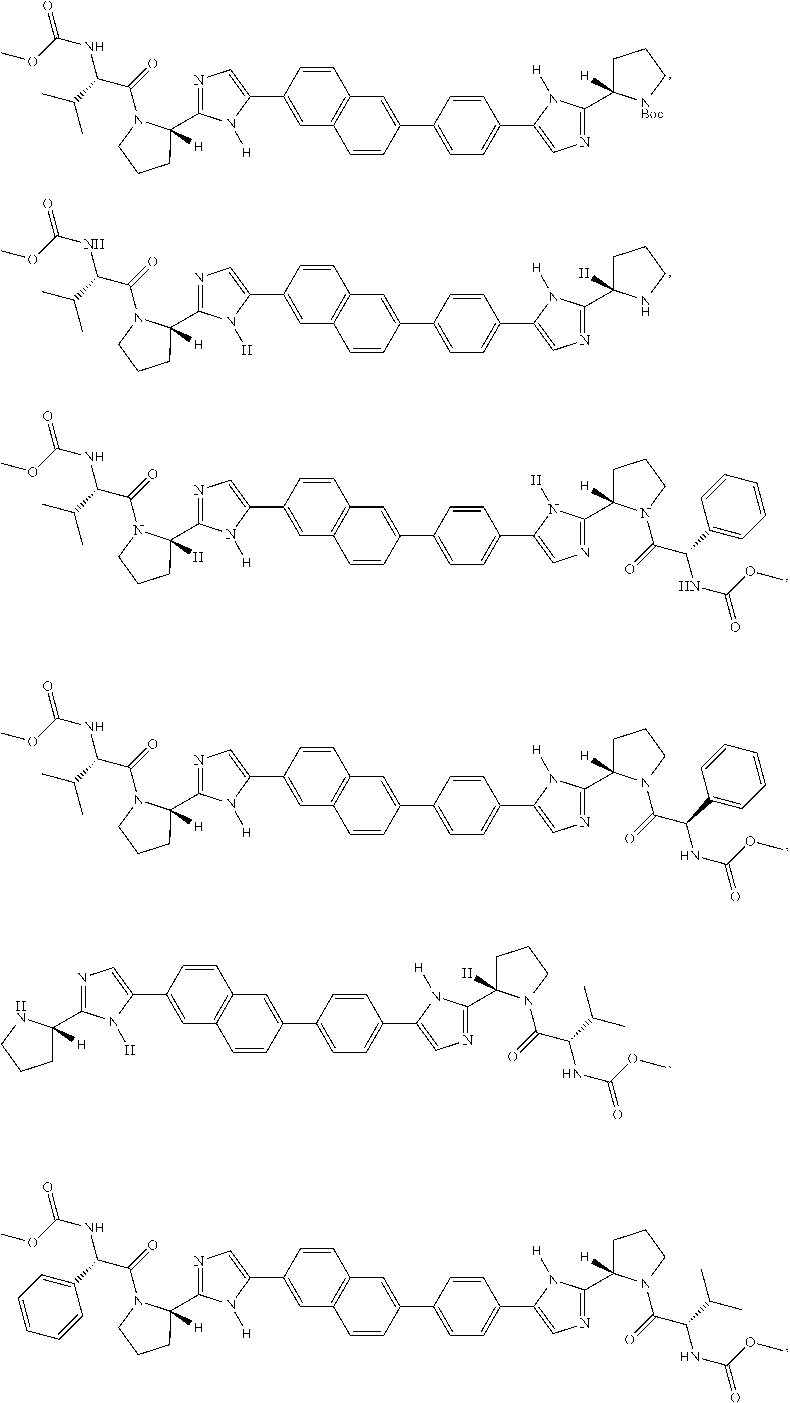

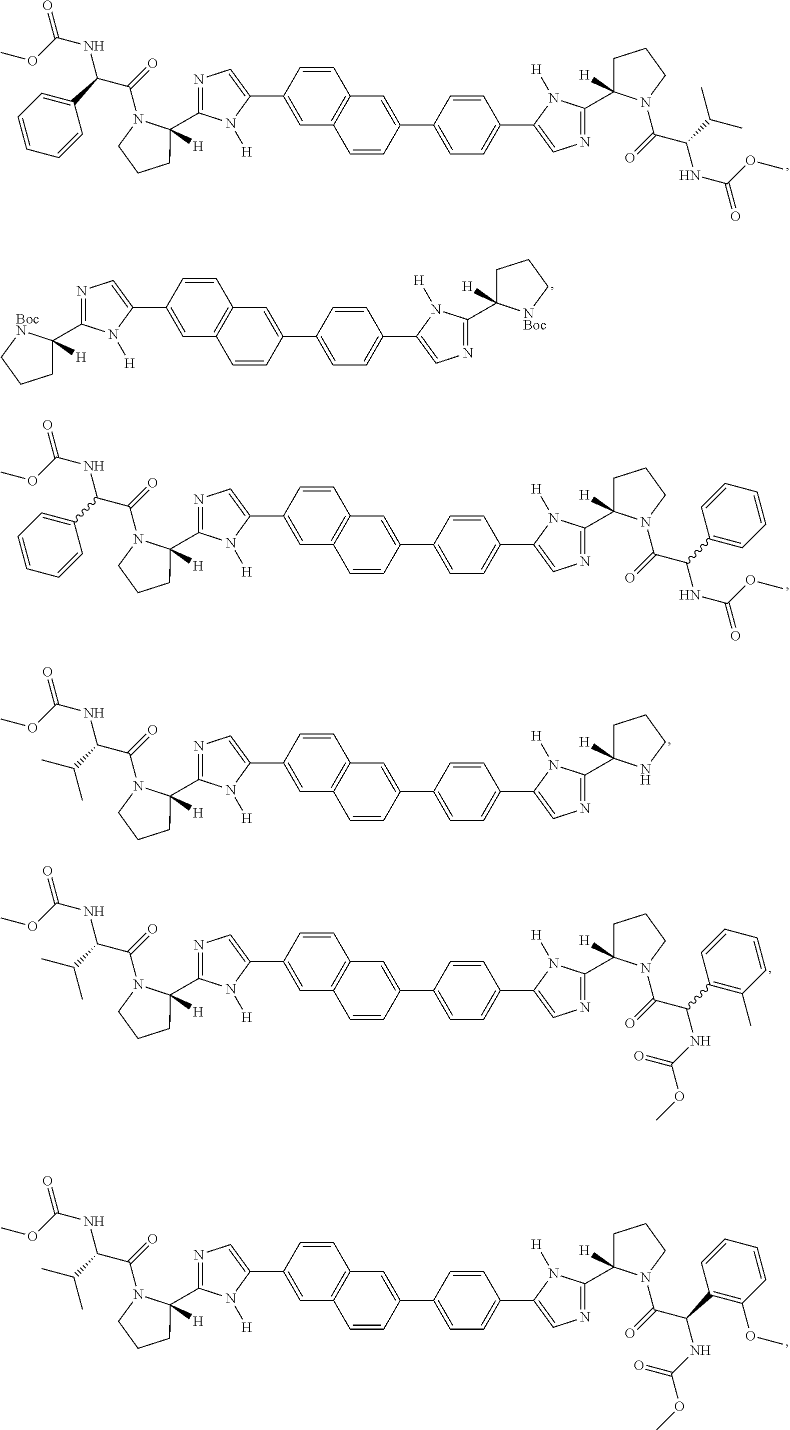

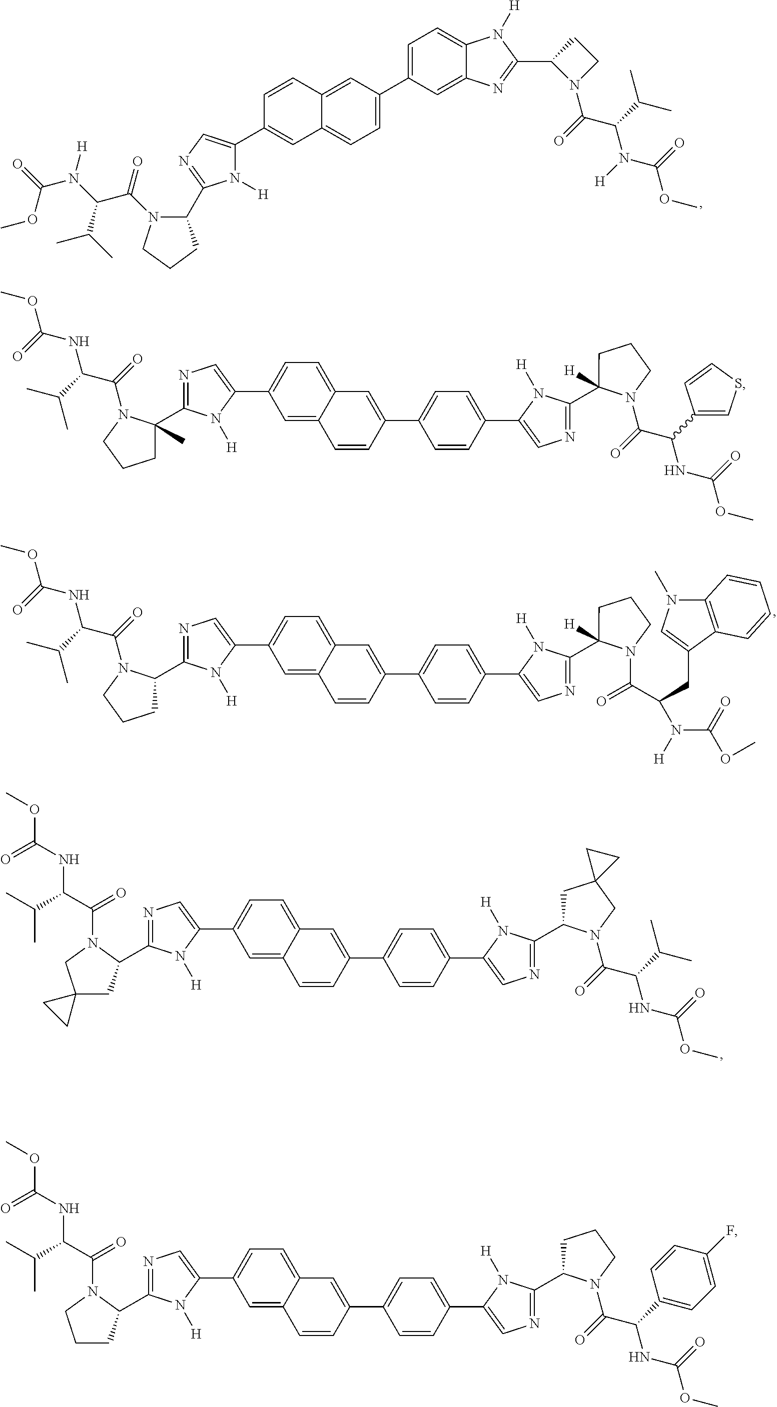

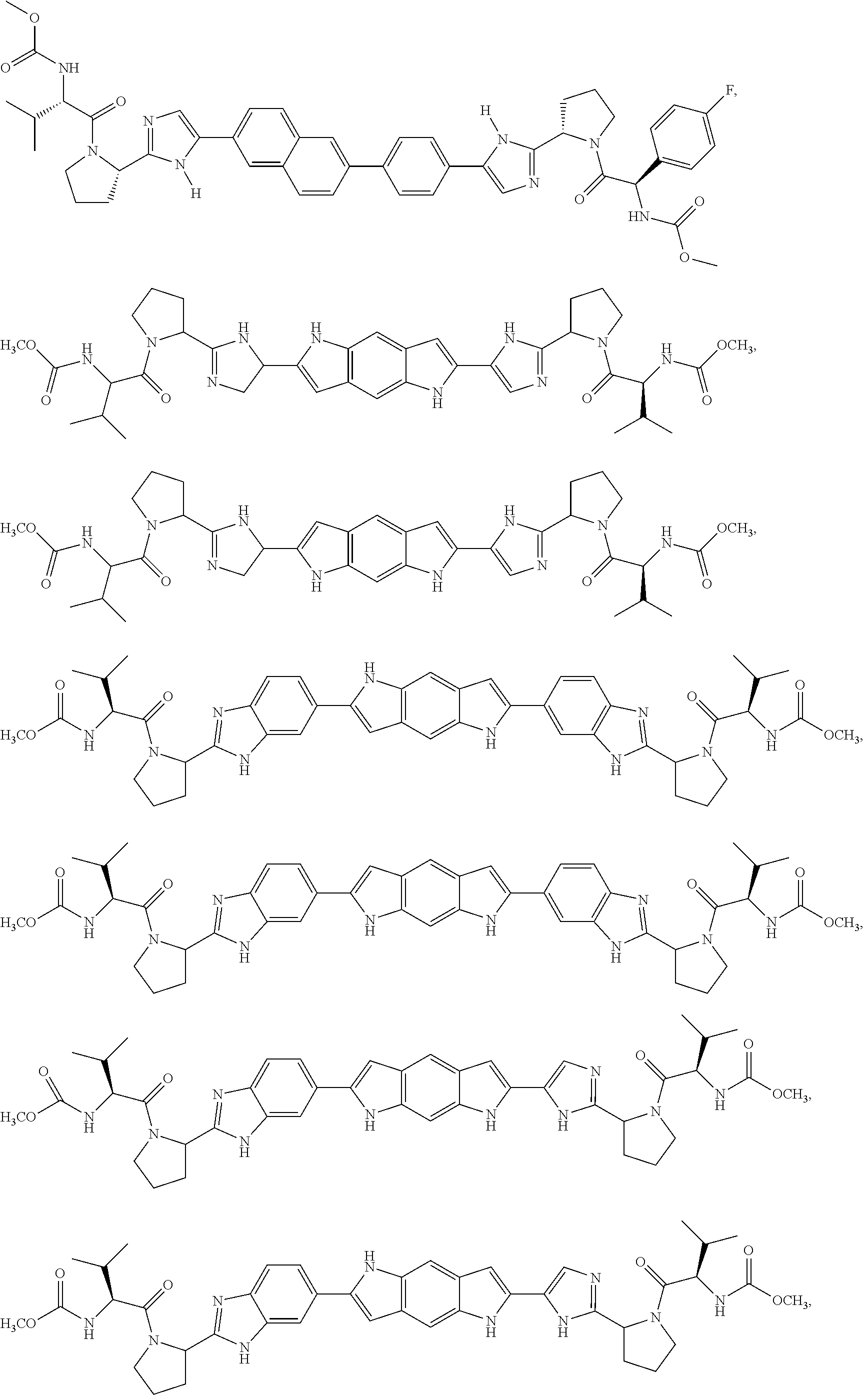

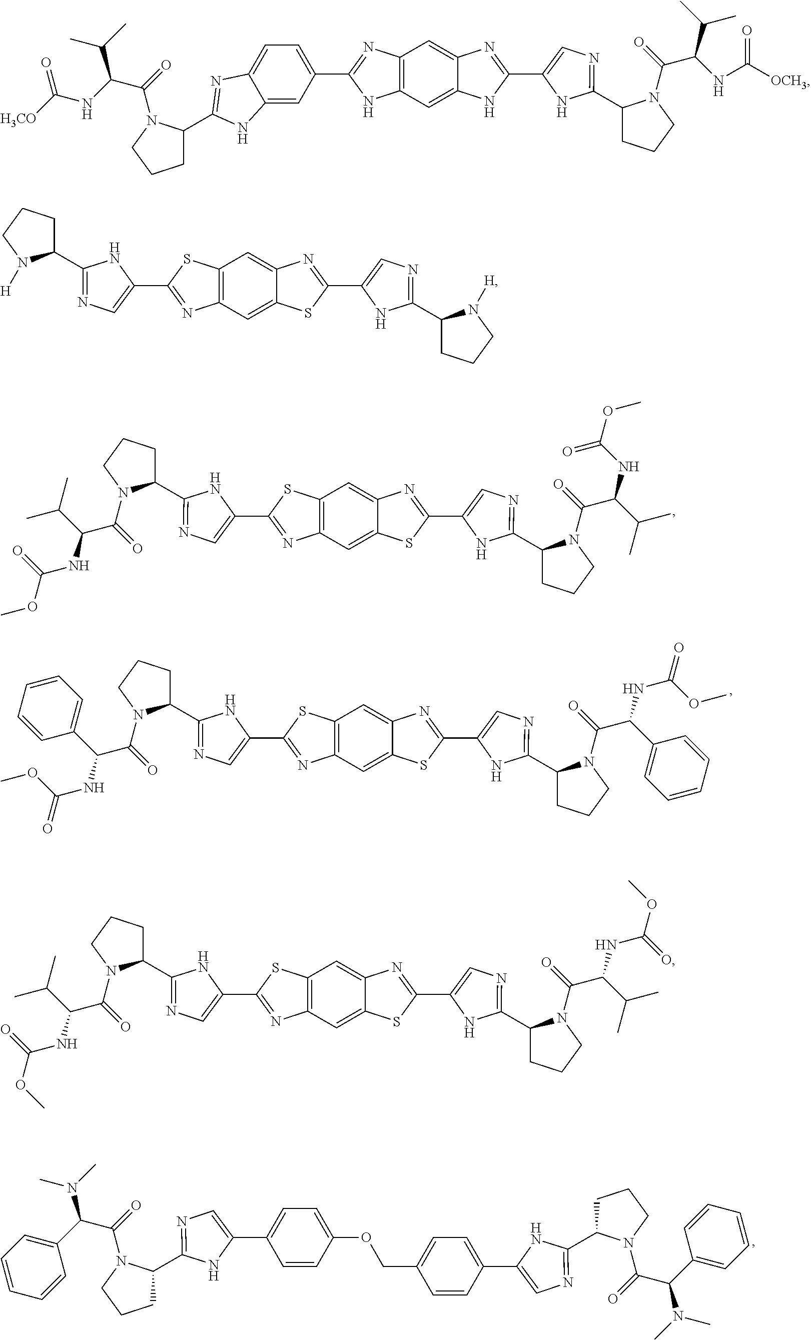

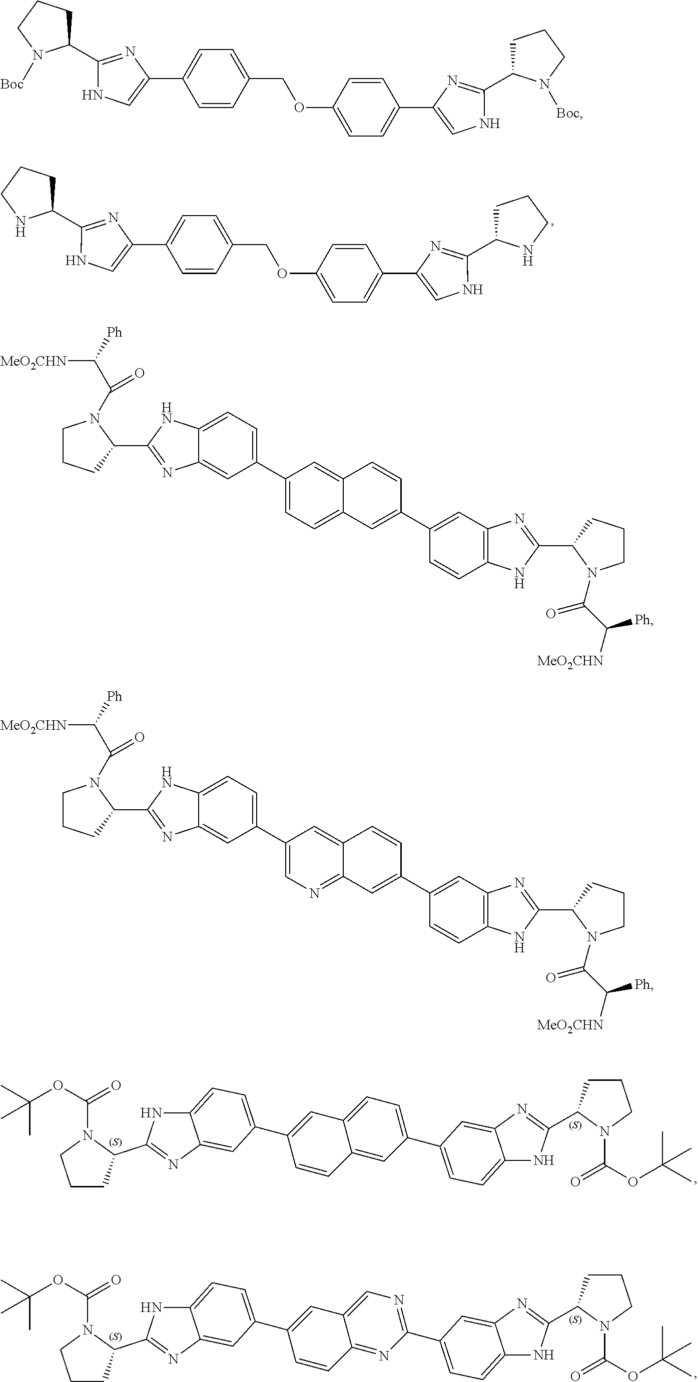

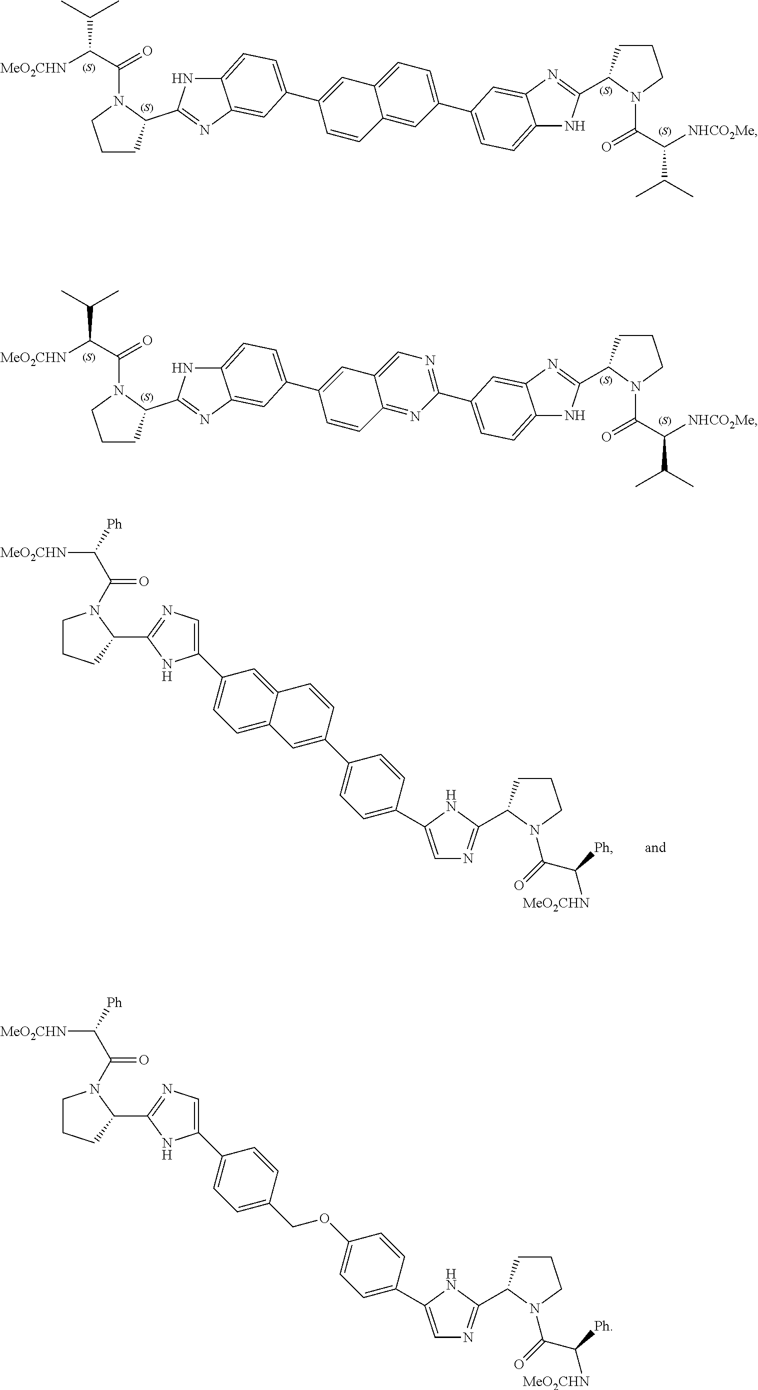

42. The compound of claim 41,
wherein:
each R^{1a} is separately selected from the group consisting of —C(R^{2a})₂NR^{3a}R^{3b}, C₁₋₆alkylOC₁₋₆alkyl, C₁₋₆alkylOC(═O)—, C₁₋₆alkylOC(═O)C₁₋₆alkyl, C₁₋₆alkylC(═O)C₁₋₆alkyl, aryl, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, (R^{c}R^{d}N)(CH═CH)ₘ—, (R^{c}R^{d}N)alkyl, (R^{c}R^{d}N)C(═O)—, C₁₋₆alkoxy optionally substituted with up to 5 halo, and C₁₋₆alkyl optionally substituted with up to 5 halo;
each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 5 halo, and C₁₋₆alkyl optionally substituted with up to 5 halo;
each R^{2a} is separately selected from the group consisting of hydrogen, C₁₋₆alkyl, aryl(CH₂)ₙ—, and heteroaryl(CH₂)ₙ—;
each R^{3a} is separately selected from the group consisting of hydrogen, and C₁₋₆alkyl;
each R^{3b} is separately selected from the group consisting of C₁₋₆alkyl, —(CH₂)ₙC(═O)NR^{4a}R^{4b}, —(CH₂)ₙC(═O)OR^{5a}, and —(CH₂)ₙC(═O)R^{6a};
each R^{4a}R^{4b}N is separately selected, wherein R^{4a} and R^{4b} are each separately selected from the group consisting of hydrogen, C₁₋₆alkyl, and aryl(CH₂)ₙ—;
each R^{5a} is separately selected from the group consisting of C₁₋₆alkyl, and aryl(CH₂)ₙ—;
each R^{6a} is separately selected from the group consisting of C₁₋₆alkyl, and aryl(CH₂)ₙ—;
X¹ is C(R²)₂, or X¹ is null;
Y¹ is selected from O (oxygen), S (sulfur), S(O), SO₂, and C(R²)₂ with the proviso that when X¹ is null Y¹ is C(R²)₂;
X² is C(R²)₂, or X² is null;
Y² is selected from O (oxygen), S (sulfur), S(O), SO₂, and C(R²)₂ with the proviso that when X² is null Y² is C(R²)₂;
each X³ is separately selected from the group consisting of NH, O (oxygen), and S (sulfur);
each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 5 halo, or optionally two vicinal R² and the carbons to which they are attached are together a fused three- to six-membered carbocyclic ring optionally substituted with up to two C₁₋₆alkyl groups;
each L¹ is separately selected from the group consisting of 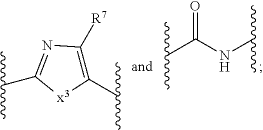
L₄ is selected from the group consisting of 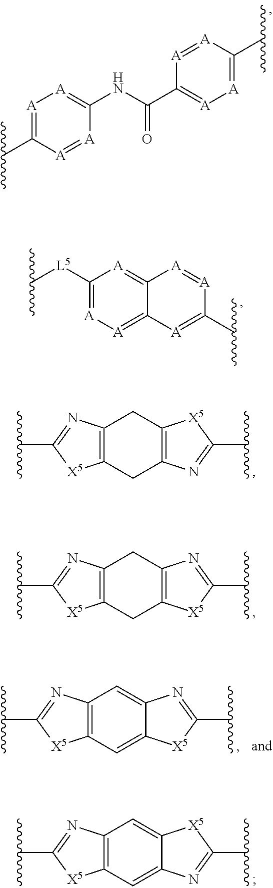
each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 5 halo and up to 5 hydroxy; and
each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 5 halo.

43. The compound of claim 41, wherein each R¹ is R^{1a}C(═O)—.

44. The compound of claim 43, wherein each R^{1a} is —CHR^{2a}NHR^{3b}.

45. The compound of claim 44, wherein each R^{2a} is C₁₋₆alkyl;
each R^{3b} is —C(═O)OR⁵; and
each R⁵ is C₁₋₆alkyl.

46. The compound of claim 41, wherein L⁴ is

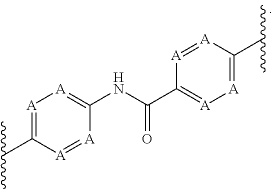

47. The compound of claim 41, wherein L⁴ is

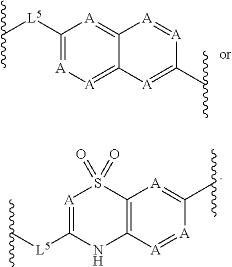

48. The compound of claim 41, wherein L⁴ is

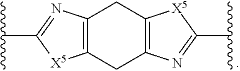

49. The compound of claim 41, wherein L⁴ is

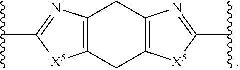

50. The compound of claim 41, wherein L⁴ is

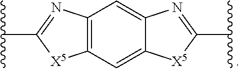

51. The compound of claim 41, wherein L⁴ is

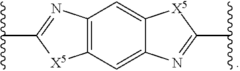

52. The compound of claim 41, wherein L⁴ is

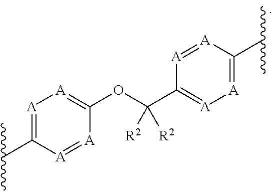

53. The compound of claim 41, wherein L⁵ is

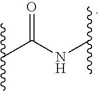

54. The compound of claim 41, wherein L⁵ is,

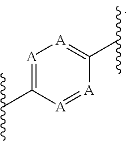

55. The compound of claim 41, wherein L⁵ is —(CH═CH)—.

56. The compound of claim 41, wherein each L³ is

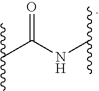

57. The compound of claim 41, having the formula: 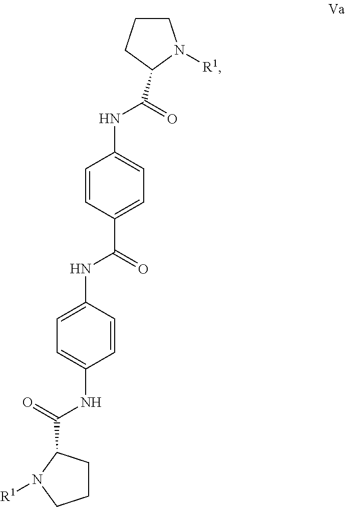
or a pharmaceutically acceptable salt thereof.

58. The compound of claim 41, having the formula: 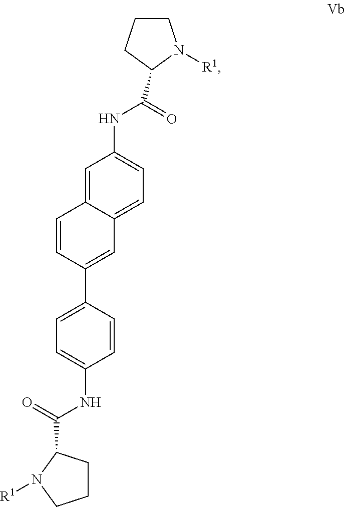
or a pharmaceutically acceptable salt thereof.

59. The compound of claim 41, having the formula: 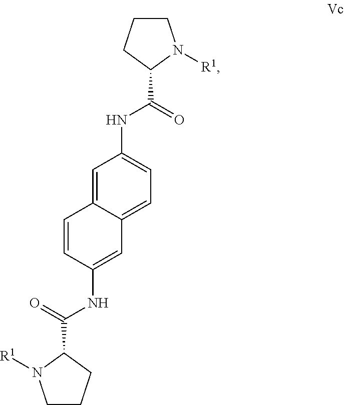
or a pharmaceutically acceptable salt thereof.

60. The compound of claim 41, having the formula: 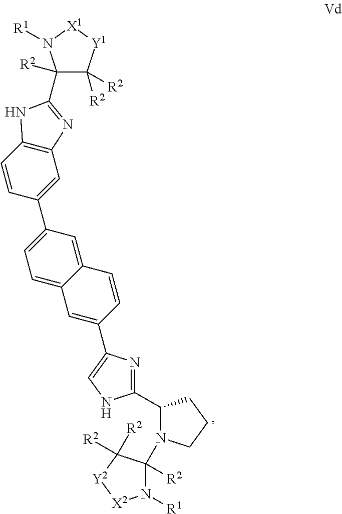
or a pharmaceutically acceptable salt thereof.

61. The compound of claim 41 having the structure of Formula Vf: 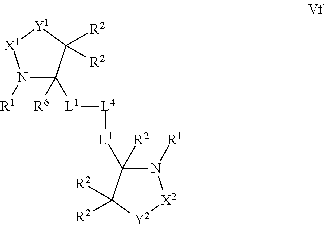
or a pharmaceutically acceptable salt thereof, wherein:
R⁶ is C₁₋₆alkyl optionally substituted with up to 9 halo.

62. The compound of claim 41, wherein L⁴ is

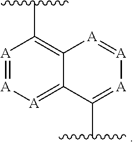

63. The compound of claim 41, wherein each L¹ is

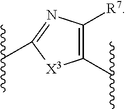

64. The compound of claim 41, wherein each L¹ is

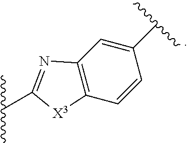

65. The compound of claim 41, wherein one L¹ is 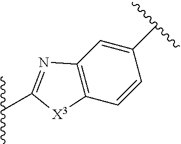
and the other L¹ is

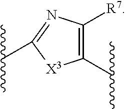

66. The compound of claim 41, wherein L⁵ is

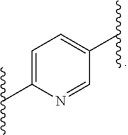

67. The compound of claim 41, having the structure: 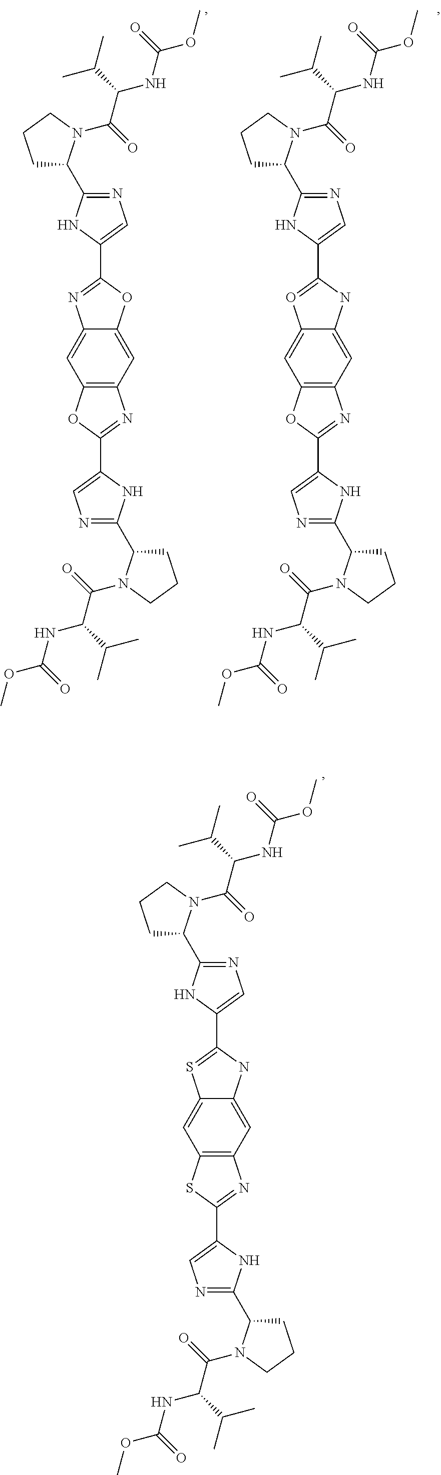 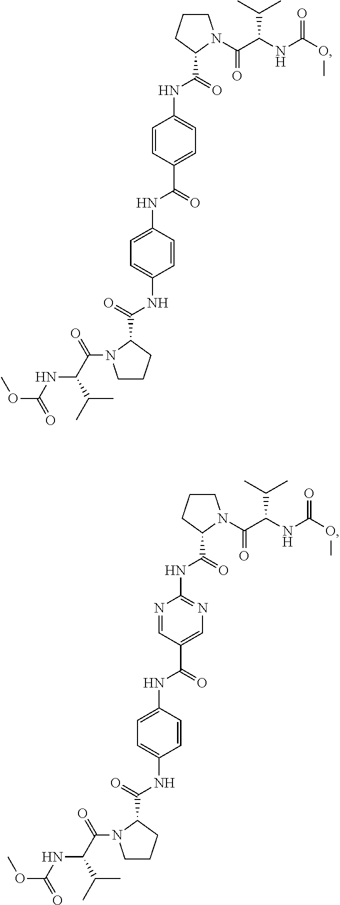 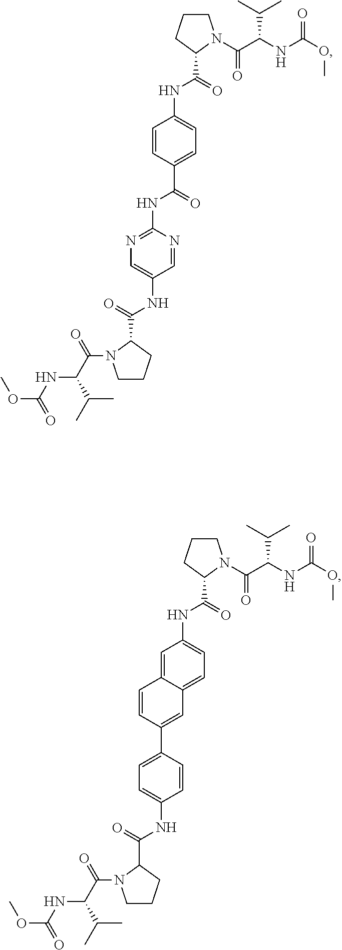 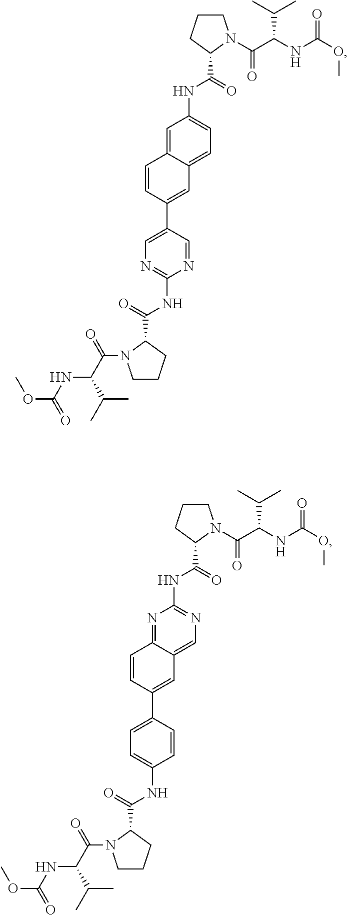 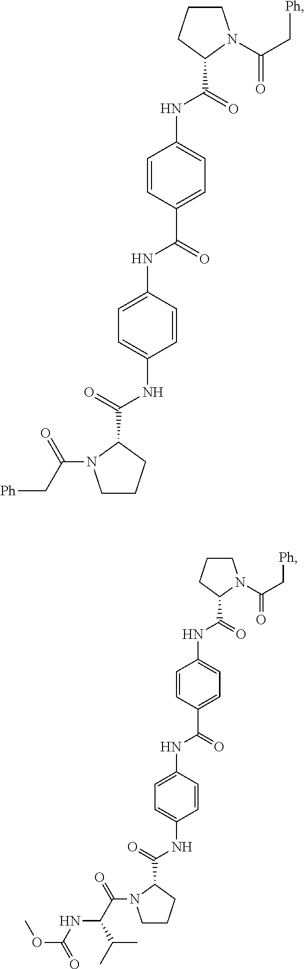 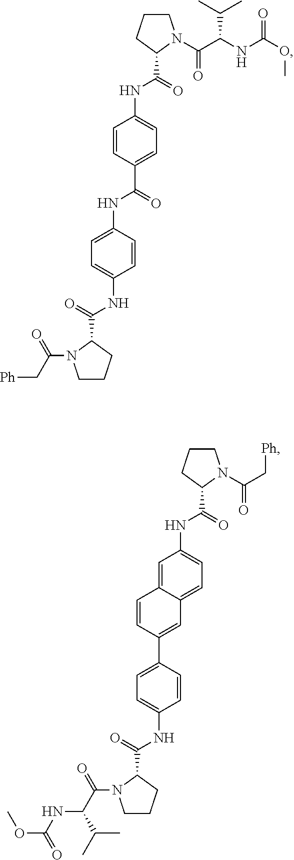 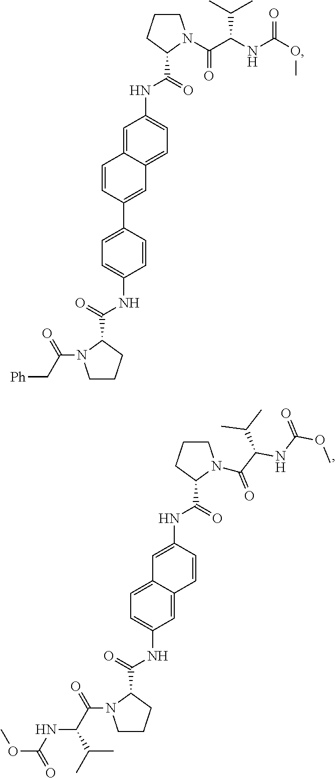 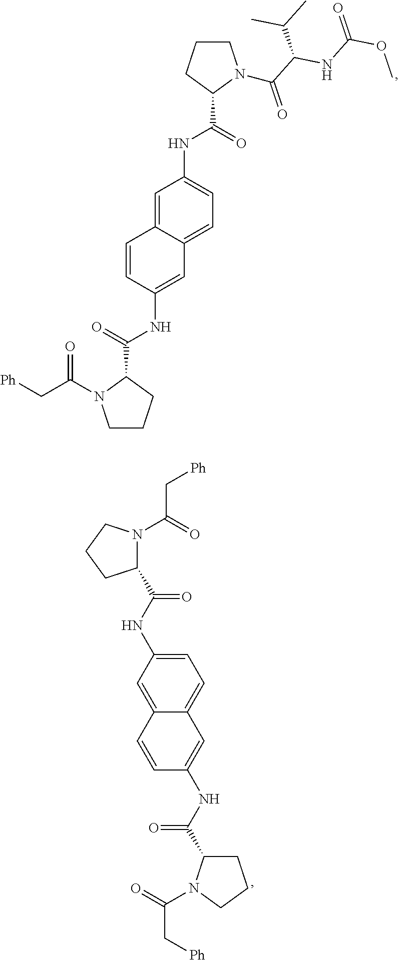 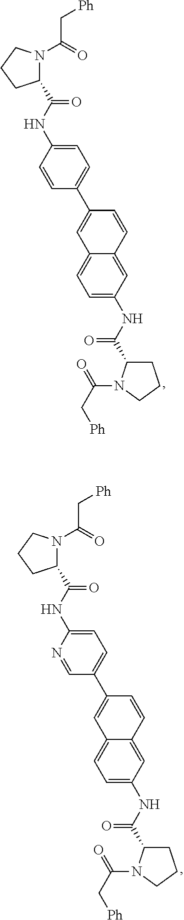 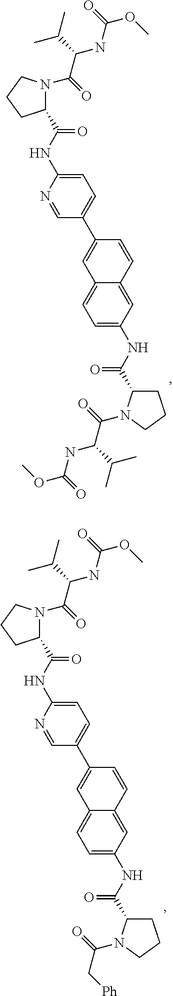 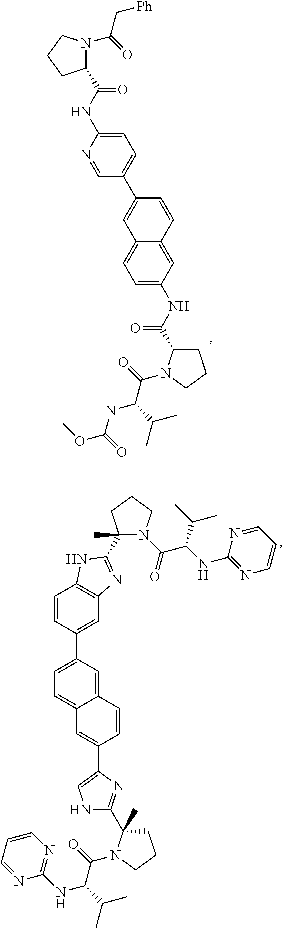 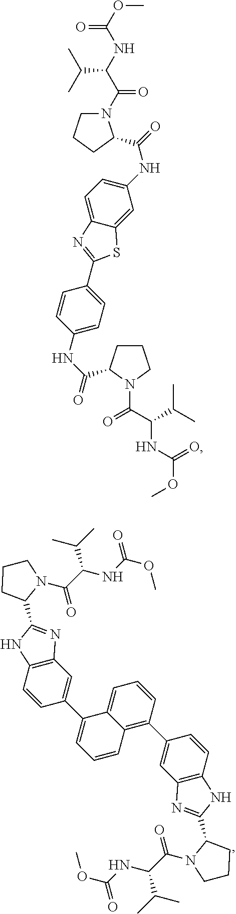 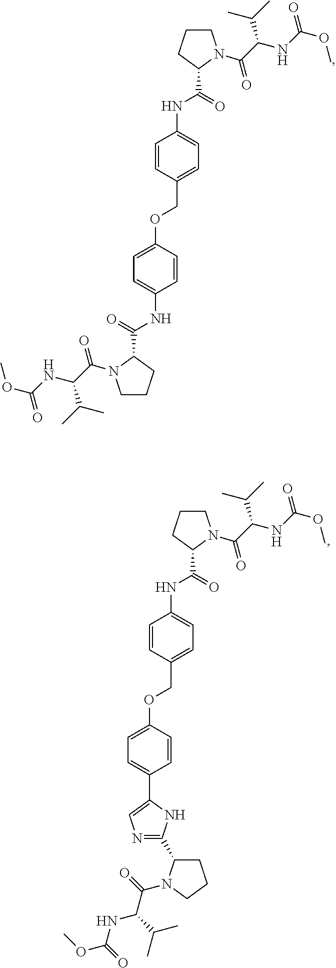 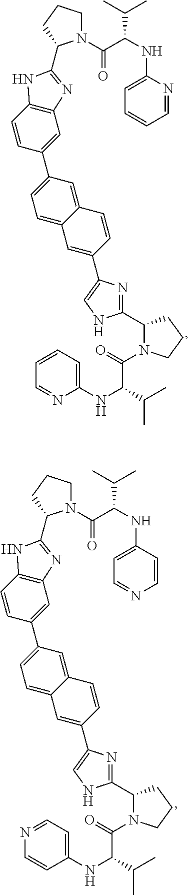 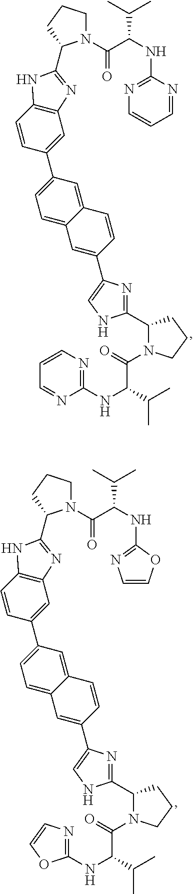 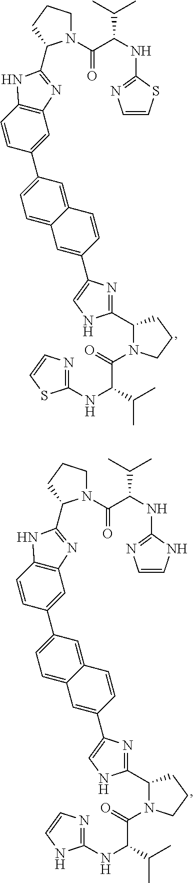 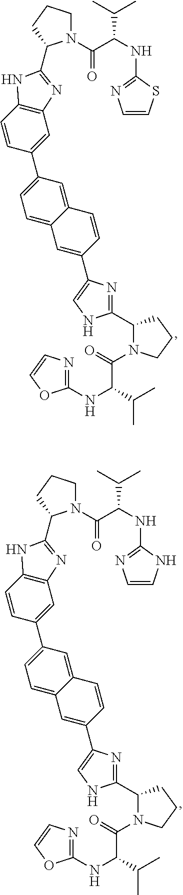 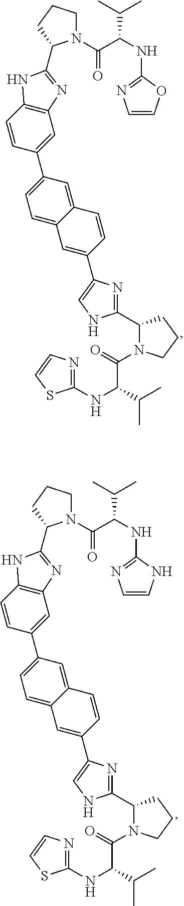 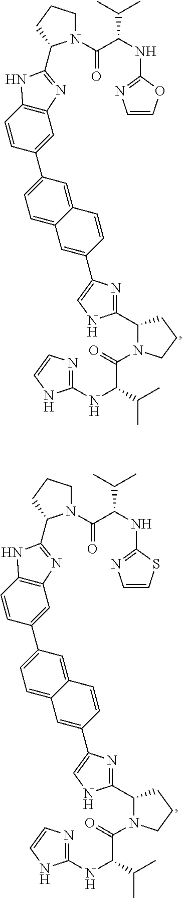 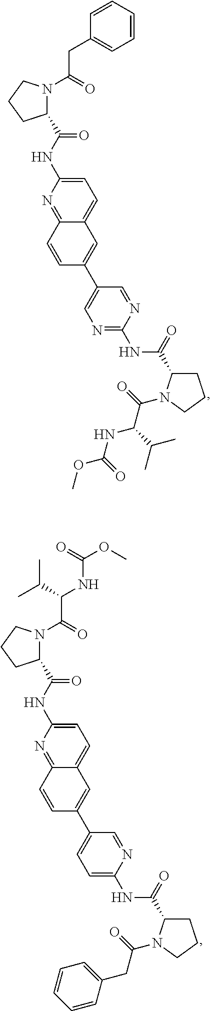 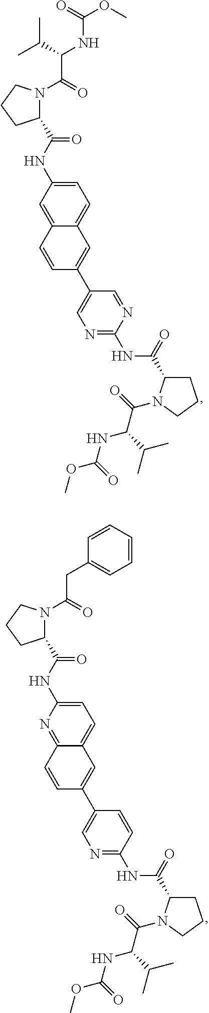 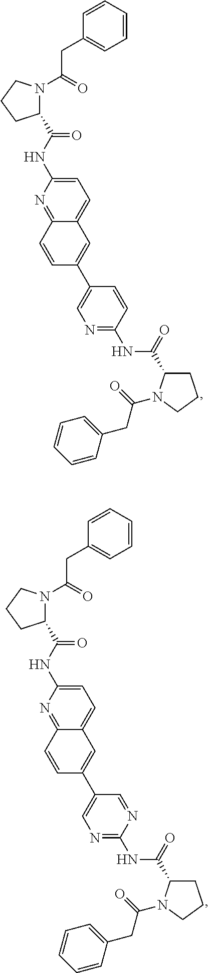 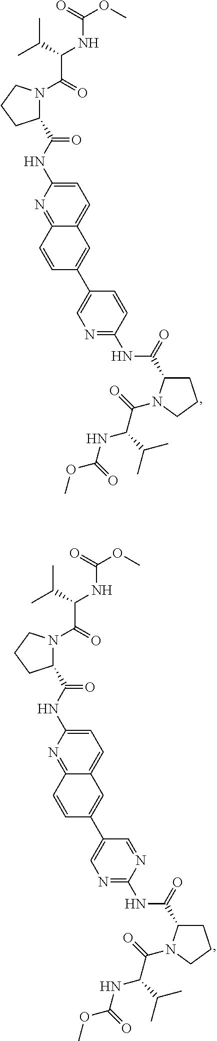 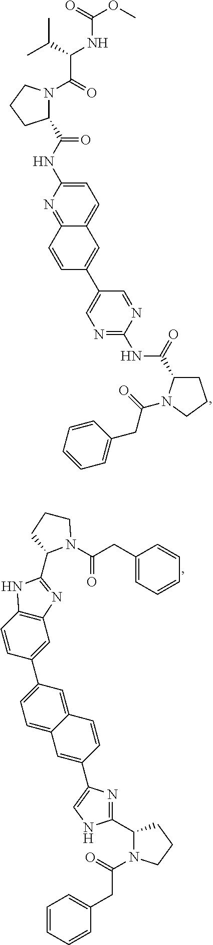 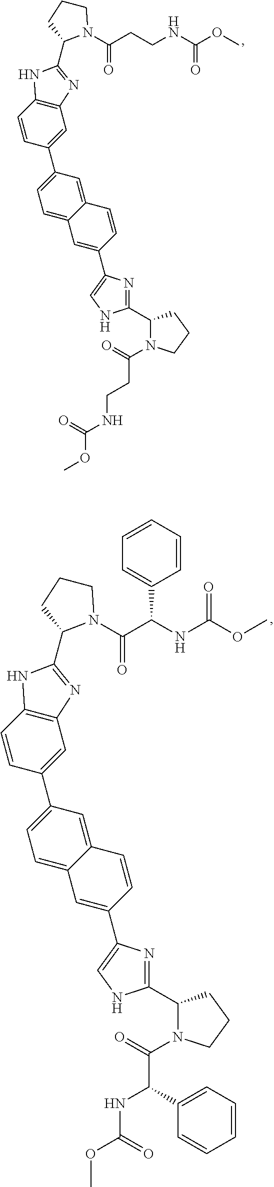 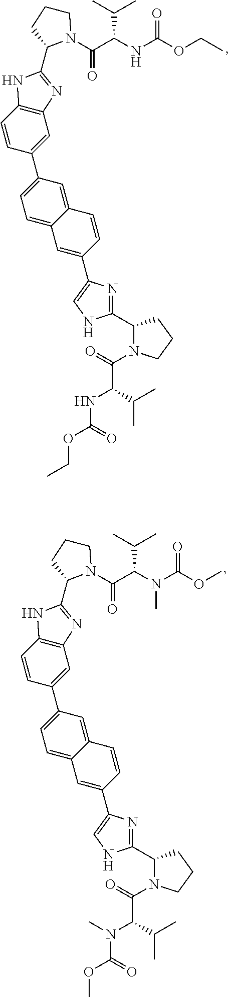 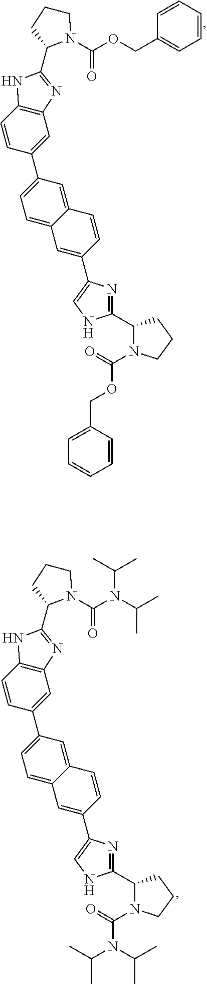 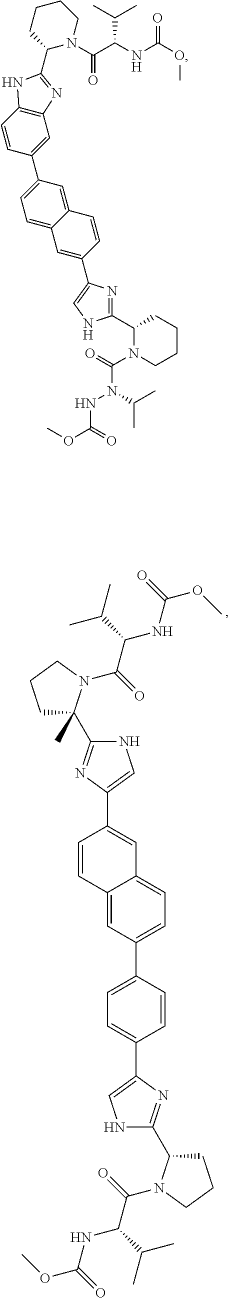 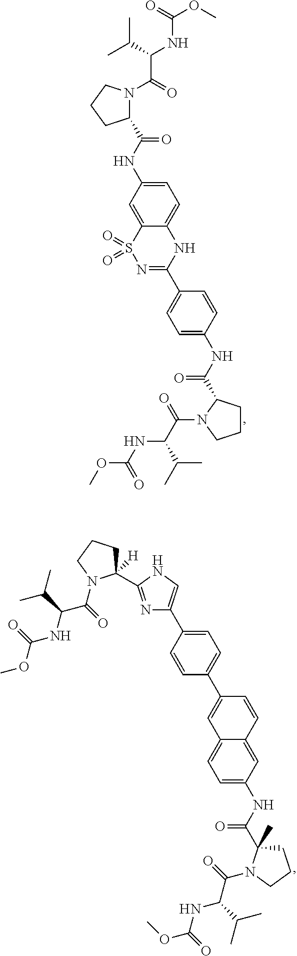 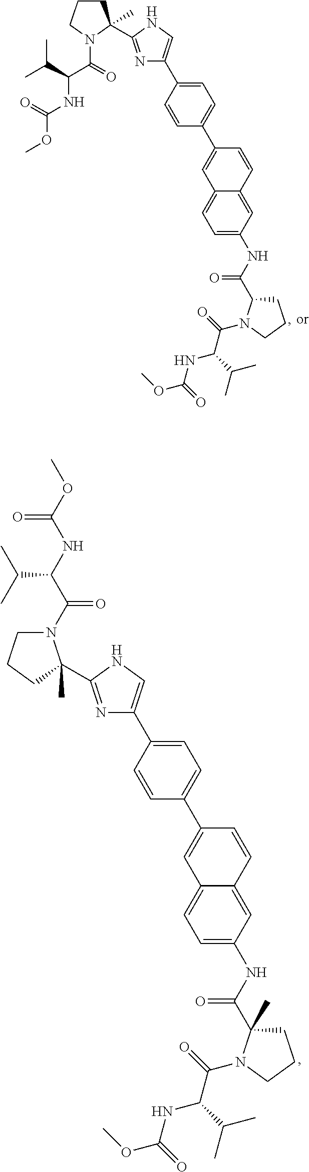
or a pharmaceutically acceptable salt thereof.

68. A pharmaceutical composition comprising a pharmaceutically acceptable excipient and a compound of claim 1.

69. A method of treating HCV infection in an individual, the method comprising administering to the individual an effective amount of a compound of claim 1.

70. The method of claim 69, further comprising identifying a subject suffering from a hepatitis C infection.

71. A method of treating liver fibrosis in an individual, the method comprising administering to the individual an effective amount of a compound of claim 1.

72. The method of claim 71, further comprising identifying a subject suffering from a hepatitis C infection.

73. A method of increasing liver function in an individual having a hepatitis C virus infection, the method comprising administering to the individual an effective amount of a compound of claim 1.

74. The method of claim 73, further comprising identifying a subject suffering from a hepatitis C infection.

75. A pharmaceutical composition comprising a pharmaceutically acceptable excipient and a compound of claim 41.

76. A method of treating HCV infection in an individual, the method comprising administering to the individual an effective amount of a compound of claim 41.

77. The method of claim 76, further comprising identifying a subject suffering from a hepatitis C infection.

78. A method of treating liver fibrosis in an individual, the method comprising administering to the individual an effective amount of a compound of claim 41.

79. The method of claim 78, further comprising identifying a subject suffering from a hepatitis C infection.

80. A method of increasing liver function in an individual having a hepatitis C virus infection, the method comprising administering to the individual an effective amount of a compound of claim 41.

81. The method of claim 80, further comprising identifying a subject suffering from a hepatitis C infection.
